(19) United States
(12) Patent Application Publication
(10) Pub. No.: US 2024/0342269 A1
(43) Pub. Date: Oct. 17, 2024

(54) OPTIMIZED NUCLEOTIDE SEQUENCES ENCODING SARS-COV-2 ANTIGENS

(71) Applicant: TRANSLATE BIO, INC., Waltham, MA (US)

(72) Inventors: Anusha DIAS, Lexington, MA (US); Khang Anh TRAN, Lexington, MA (US); Minnie ZACHARIA, Lexington, MA (US); Xiaobo GU, Lexington, MA (US); Lianne BOEGLIN, Lexington, MA (US); Joseph A. SKALESKI, Lexington, MA (US); Shrirang KARVE, Lexington, MA (US); Frank DEROSA, Lexington, MA (US); Tong-Ming FU, Lexington, MA (US); Kirill KALNIN, Lexington, MA (US); Sudha CHIVUKULA, Lexington, MA (US); Timothy PLITNIK, Lexington, MA (US); Danilo CASIMIRO, Lexington, MA (US); Jeffrey S. DUBINS, Lexington, MA (US)

(21) Appl. No.: 17/923,497

(22) PCT Filed: May 7, 2021
(86) PCT No.: PCT/US2021/031256

§ 371 Date: May 7, 2024

Related U.S. Application Data
(60) Provisional application No. 63/021,319, filed on May 7, 2020.
(60) Provisional application No. 63/032,825, filed on Jun. 1, 2020.
(60) Provisional application No. 63/076,729, filed on Sep. 10, 2020.
(60) Provisional application No. 63/076,718, filed on Sep. 10, 2020.
(60) Provisional application No. 63/088,739, filed on Oct. 7, 2020.
(60) Provisional application No. 63/143,604, filed on Jan. 29, 2021.
(60) Provisional application No. 63/143,612, filed on Jan. 29, 2021.
(60) Provisional application No. 63/146,807, filed on Feb. 8, 2021.

(51) Int. Cl. A61K 39/215; A61K 9/51; A61K 39/00; A61P 31/14; C07K 14/005

(52) U.S. Cl. CPC A61K 39/215; A61K 9/5123; A61P 31/14; C07K 14/005; A61K 2039/53; C12N 2770/20022; C12N 2770/20034

(57) ABSTRACT

The present invention relates to optimized nucleotide sequence encoding SARS-COV-2 antigens. These sequences are particularly suitable for use in vaccine compositions for the treatment or prevention of infections caused by a β-coronaviruses, including COVID-19 infections, in a human or animal subject in need of such treatment.

### CROSS-REFERENCE TO RELATED APPLICATIONS

This application is a 35 U.S.C. § 371 National Stage Application of International Application No. PCT/US2021/031256, filed on May 7, 2021, which claims benefit of, and priority to U.S. Provisional Patent Application Ser. No. 63/021,319 filed on May 7, 2020, U.S. Provisional Patent Application Ser. No. 63/032,825 filed on Jun. 1, 2020, U.S. Provisional Patent Application Ser. No. 63/076,718 filed on Sep. 10, 2020, U.S. Provisional Patent Application Ser. No. 63/076,729 filed on Sep. 10, 2020, U.S. Provisional Patent Application Ser. No. 63/088,739 filed on Oct. 7, 2020, U.S. Provisional Patent Application Ser. No. 63/143,604 filed on Jan. 29, 2021, U.S. Provisional Patent Application Ser. No. 63/143,612 filed on Jan. 29, 2021, and U.S. Provisional Patent Application Ser. No. 63/146,807 filed on Feb. 8, 2021. The contents of each of the foregoing applications are hereby incorporated by reference in their entireties.

### SEQUENCE LISTING

The present specification makes reference to a Sequence Listing electronically filed in ASCII format. The sequence listing file named 122548. US044_ST25.txt, was created on Apr. 3, 2024, and is 811,008 bytes in size. The entire contents of the sequence listing are herein incorporated by reference in their entirety.

### FIELD OF THE INVENTION

The present invention relates to SARS-COV-2 antigenic polypeptides and to optimized nucleotide sequence encoding these SARS-COV-2 antigenic polypeptides. These antigenic polypeptides and optimized nucleotide sequences are particularly suitable for use in vaccine compositions for the treatment or prevention of infections caused by a β-coronaviruses, including COVID-19 infections, in a human or animal subject in need of such treatment.

### BACKGROUND OF THE INVENTION

The Coronavirus Disease 2019 (COVID-19) pandemic poses a serious threat to global public health. The causative agent of COVID-19 is severe acute respiratory syndrome coronavirus 2 (SARS-COV-2), a newly emerged human pathogen.

Protein antigen selection and design both contribute to the immunogenicity of a vaccine, whether it is protein-based or nucleic acid-based. Moreover, with respect to nucleic acid-based immunogenic compositions such as mRNA-based vaccines, expression levels achieved from the nucleic acid encoding one or more protein antigens can significantly impact efficacy.

Recombinant DNA technology and advances in nucleic acid sequencing and synthesis have made it possible to rapidly design protein antigens, once the genome sequence of a pathogen has been determined. Success or failure of a vaccine can depend on the selection of antigenic polypeptides that yield a highly effective response in form of neutralizing antibodies in vivo. Therefore a need exists to provide new antigenic polypeptides derived from SARS-COV-2 proteins for use in immunogenic compositions that provide prophylaxis against COVID-19.

Effective expression or production of a protein from an mRNA within a cell depends on a variety of factors. Optimization of the composition and order of codons within a protein-coding nucleotide sequence (“codon optimization”) can lead to higher expression of the mRNA-encoded protein. Various methods of performing codon optimization are known in the art, however, each has significant drawbacks and limitations from a computational and/or therapeutic point of view. In particular, known methods of codon optimization often involve, for each amino acid, replacing every codon with the codon having the highest usage for that amino acid, such that the “optimized” sequence contains only one codon encoding each amino acid.

Accordingly, a need exists for improved codon optimization methods that generate an optimized nucleotide sequence for increased expression of mRNA encoding a selected or designed protein antigen for the production of an efficacious mRNA vaccine.

Moreover, with the global spread of SARS-COV-2, new variants of the virus have emerged. Therefore, a need exists to provide pharmaceutical compositions (e.g., immunogenic compositions) that are capable of eliciting a broadly neutralizing antibody response effective against a multitude of naturally occurring variants of SARS-COV-2.

### SUMMARY OF THE INVENTION

The present invention addresses the need for selecting and/or designing a protein antigen that yields an effective immune response against SARS-COV-2. It also addressed the need for generating optimized nucleotide sequences encoding that protein antigen for the effective treatment or prevention of COVID-19 infections through the provision of a vaccine comprising a nucleic acid (e.g., an mRNA) with the optimized nucleotide sequence. Various selected and/or designed protein antigens against SARS-COV-2 are provided herein, as well as at least one optimized nucleotide sequence for each such protein antigen.

In addition, a method is provided for analyzing an amino acid sequence of a protein antigen to produce at least one optimized nucleotide sequence. The optimized nucleotide sequence for each selected and/or designed protein antigen is designed to increase the expression of that encoded protein antigen compared to the expression of the protein associated with a naturally occurring nucleotide sequence. Codon optimization produces a protein-coding nucleotide sequence based on various criteria without altering the sequence of translated amino acids of the encoded protein antigen, due to the redundancy in the genetic code. Moreover, the optimized nucleotide sequences disclosed here are designed to produce high-quality full-length transcripts during in vitro synthesis and therefore can be manufactured more cost effectively than optimized nucleotide sequences generated with prior art codon optimization algorithms. In particular, termination sequences and the like that could result in incomplete transcripts during in vitro synthesis are effectively removed by the sequence optimization processes described herein.

As demonstrated in the examples, immunogenic compositions that comprise a LNP-encapsulated optimized nucleotide sequence of the invention which encodes a full-length pre-fusion stabilized SARS-COV-2 S protein can produce an effective neutralizing antibody response and therefore can provide protective efficacy against COVID-19 infection.

The present invention also addresses the need for immunogenic compositions that are capable of eliciting a broadly effective immune response, in particular in the form of neutralizing antibodies, against naturally occurring variants of SARS-COV-2. As shown in the examples, the inventors surprisingly discovered that administration of an immunogenic composition that comprises a LNP-encapsulated optimized nucleotide sequence which encodes a South African variant of the SARS-COV-2 S protein to subjects who have been previously immunized with a COVID-19 vaccine can induce an effective neutralizing antibody response against a broad range of β-coronaviruses, including naturally occurring variants of SARS-COV-2 isolated in Wuhan, South Africa, Japan/Brazil and California, as well as the phylogenetically more distant SARS-CoV-1 strain.

In particular, the invention provides a nucleic acid comprising an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline, wherein the optimized nucleotide sequence consists of codons associated with a usage frequency which is greater than or equal to 10%; wherein the optimized nucleotide sequence: does not contain a termination signal having one of the following nucleotide sequences: 5′-X₁ATCTX₂TX₃-3′, wherein X₁, X₂ and X₃ are independently selected from A, C, T or G; and 5′-X₁AUCUX₂UX₃-3′, wherein X₁, X₂ and X₃ are independently selected from A, C, U or G; does not contain any negative cis-regulatory elements and negative repeat elements; and has a codon adaptation index greater than 0.8; wherein, when divided into non-overlapping 30 nucleotide-long portions, each portion of the optimized nucleotide sequence has a guanine cytosine content range of 30%-70%. In particular embodiments, the nucleic acid is mRNA. In some embodiments, the nucleic acid is DNA. In certain embodiments, the optimized nucleotide sequence does not contain a termination signal having one of the following sequences: TATCTGTT; TTTTTT; AAGCTT; GAAGAGC; TCTAGA; UAUCUGUU; UUUUUU; AAGCUU; GAAGAGC; UCUAGA.

In some embodiments, the optimized nucleotide sequence encodes the amino acid sequence of SEQ ID NO:11. In particular embodiments, the optimized nucleotide sequence is at least 85% (e.g., at least 90%) identical to the nucleic acid sequence of SEQ ID NO: 44 or SEQ ID NO: 148 and encodes the amino acid sequence of SEQ ID NO: 11. In specific embodiments, the optimized nucleotide sequence has the nucleic acid sequence of SEQ ID NO: 44 or SEQ ID NO: 148.

In some embodiments, the full-length SARS-COV-2 spike protein encoded by the optimized sequence further contains the L18F, D80A, D215G, L242-, A243-, L244-, K417N, E484K, N501Y, D614G and A701V mutations. In these embodiments, the optimized nucleotide sequence may encode the amino acid sequence of SEQ ID NO: 167. In particular embodiments, the optimized nucleotide is at least 85% (e.g., at least 90%) identical to the nucleic acid sequence of SEQ ID NO: 166 or SEQ ID NO: 173 and encodes the amino acid sequence of SEQ ID NO: 167. In specific embodiments, the optimized nucleotide sequence has the nucleic acid sequence of SEQ ID NO: 166 or SEQ ID NO: 173.

In certain embodiments, a nucleic acid of the invention is for use in therapy. For example, the invention also provides an immunogenic composition comprising a nucleic acid of the invention for use in the prophylaxis of an infection caused by a β-coronavirus. In addition, the invention also provides use of a nucleic acid of the invention in the manufacture of a medicament for the prophylaxis of an infection caused by a β-coronavirus. In certain embodiments, the β-coronavirus expresses a spike protein which binds to angiotensin-converting enzyme 2 (ACE2). In a specific embodiment, the β-coronavirus is SARS-COV-2. In other embodiments, the β-coronavirus has a spike protein that is at least 75%, 80%, 90%, 95% or 99% identical to SEQ ID NO: 1.

The invention further provides a method of treating or preventing an infection caused by a β-coronavirus, said method comprising administering to a subject an effective amount of an immunogenic composition comprising a nucleic acid of the invention. In certain embodiments, the β-coronavirus expresses a spike protein which binds to angiotensin-converting enzyme 2 (ACE2). In a specific embodiments, the β-coronavirus is SARS-COV-2. In other embodiments, the β-coronavirus has a spike protein that is at least 75%, 80%, 90%, 95% or 99% identical to SEQ ID NO: 1.

Furthermore, the invention provides a pharmaceutical composition comprising i) a nucleic acid of the invention and ii) a lipid nanoparticle. In certain embodiments, the nucleic acid is mRNA, which may be present at a concentration of between about 0.5 mg/mL to about 1.0 mg/mL. In certain embodiments, the nucleic acid of the invention (e.g., an mRNA in accordance with the invention) is encapsulated in the lipid nanoparticle. In certain embodiments, the lipid nanoparticle comprises a cationic lipid, a non-cationic lipid, a cholesterol-based lipid, and a PEG-modified lipid. In particular embodiments, the cationic lipid is selected from cKK-E12, cKK-E10, OF-Deg-Lin and OF-02; the non-cationic lipid is selected from DOPE and DEPE; the cholesterol-based lipid is cholesterol; and the PEG-modified lipid is DMG-PEG-2K. In certain embodiments, the cationic lipid constitutes about 30-60% of the lipid nanoparticle by molar ratio, e.g., about 35-40%. In certain embodiments, the ratio of cationic lipid to non-cationic lipid to cholesterol-based lipid to PEG-modified lipid is approximately 30-60:25-35:20-30:1-15 by molar ratio.

In certain embodiments, a lipid nanoparticle encapsulating a nucleic acid of the invention (e.g., an mRNA in accordance with the invention) comprises cKK-E12, DOPE, cholesterol and DMG-PEG2K; cKK-E10, DOPE, cholesterol and DMG-PEG2K; OF-Deg-Lin, DOPE, cholesterol and DMG-PEG2K; or OF-02, DOPE, cholesterol and DMG-PEG2K. In a specific embodiment, the lipid nanoparticle comprises cKK-E10, DOPE, cholesterol and DMG-PEG2K at the molar ratios 40:30:28.5:1.5. In certain embodiments, the lipid nanoparticle has an average size of less than 150 nm, e.g., less than 130 nm, less than 110 nm, less than 100 nm. In some embodiments, the lipid nanoparticle has an average size of about 90-110 nm, or has an average size of about 50-70 nm, e.g., about 55-65 nm.

In certain embodiments, a pharmaceutical composition of the invention is for use in treating or preventing an infection caused by a β-coronavirus. In certain embodiments, the β-coronavirus expresses a spike protein which binds to angiotensin-converting enzyme 2 (ACE2). In a specific embodiment, the β-coronavirus is SARS-COV-2. In other embodiments, the β-coronavirus has a spike protein that is at least 75%, 80%, 90%, 95% or 99% identical to SEQ ID NO: 1.

In certain embodiments, a pharmaceutical composition of the invention is administered intramuscularly. In certain embodiments, a pharmaceutical composition of the invention is administered at least once. In some embodiments, a pharmaceutical composition is administered at least twice. In particular embodiments, the period between administrations is at least 2 weeks, e.g. 3 weeks, or 1 month. In some embodiments, the period between administrations is about 3 weeks.

In one particular embodiment, the invention provides an mRNA construct (mRNA construct 1) consisting of the following structural elements:
a 5′ cap with the following structure:

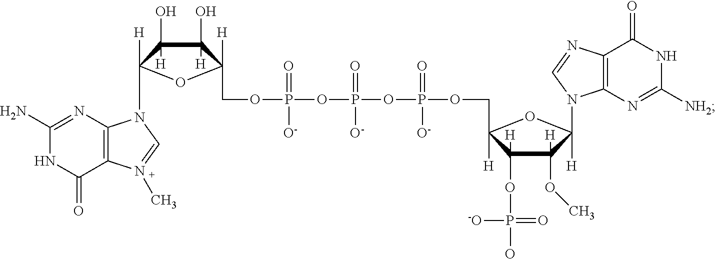

a 5′ untranslated region (5′ UTR) having the nucleic acid sequence of SEQ ID NO: 144;
a protein coding region having the nucleic acid sequence of SEQ ID NO: 148;
a 3′ untranslated region (3′ UTR) having the nucleic acid sequence of SEQ ID NO: 145; and
a poly A tail.

In another particular embodiment, the invention provides an mRNA construct (mRNA construct 2) consisting of the following structural elements:
a 5′ cap with the following structure:

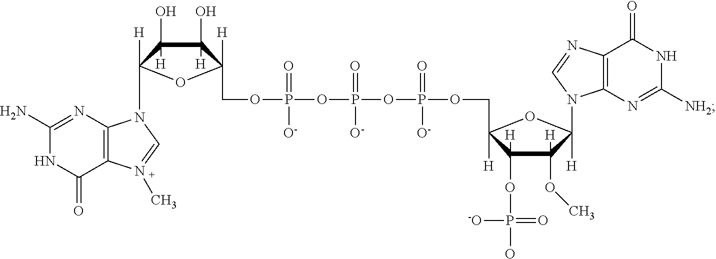

a 5′ untranslated region (5′ UTR) having the nucleic acid sequence of SEQ ID NO: 144;
a protein coding region having the nucleic acid sequence of SEQ ID NO: 173;
a 3′ untranslated region (3′ UTR) having the nucleic acid sequence of SEQ ID NO: 145; and
a poly A tail.

In specific embodiments, the invention provides a lipid nanoparticle encapsulating an mRNA construct of the invention. In some embodiments, the lipid nanoparticle encapsulates more than one mRNA construct of the invention, e.g. a lipid nanoparticle may encapsulate both mRNA construct 1 and mRNA construct 2. In certain embodiments, the lipid nanoparticle comprises a cationic lipid, a non-cationic lipid, a cholesterol-based lipid and a PEG-modified lipid. In certain embodiments, the cationic lipid is selected from cKK-E12, cKK-E10, OF-Deg-Lin and OF-02; the non-cationic lipid is selected from DOPE and DEPE; the cholesterol-based lipid is cholesterol; and the PEG-modified lipid is DMG-PEG-2K. In a specific embodiment, the lipid nanoparticle comprises cKK-E10, DOPE, cholesterol and DMG-PEG2K at the molar ratios 40:30:28.5:1.5.

The invention also provides an immunogenic composition comprising an mRNA construct of the invention, or a lipid nanoparticle encapsulating an mRNA construct of the invention. In some embodiments, the immunogenic composition comprises more than one mRNA constructs of the invention, e.g., mRNA construct 1 and mRNA construct 2. In some embodiments, the immunogenic composition comprises the more than one mRNA constructs (e.g., mRNA construct 1 and mRNA construct 2) encapsulated in the same lipid nanoparticle. In other embodiments, the more than one mRNA constructs e.g., mRNA construct 1 and mRNA construct 2) are encapsulated in separate lipid nanoparticles. In certain embodiments, the immunogenic composition comprises between 5 μg and 200 μg of the mRNA construct(s).

In certain embodiments, the immunogenic composition comprises between 7 μg and 135 μg of the mRNA construct(s). In certain embodiments, the immunogenic composition comprises at least 10 μg of the mRNA construct(s). In certain embodiments, the immunogenic composition comprises at least 15 μg of the mRNA construct(s). In certain embodiments, the immunogenic composition comprises at least 20 μg of the mRNA construct(s). In certain embodiments, the immunogenic composition comprises at least 25 μg of the mRNA construct(s). In certain embodiments, the immunogenic composition comprises at least 35 μg of the mRNA construct(s). In certain embodiments, the immunogenic composition comprises at least 40 μg of the mRNA construct(s). In certain embodiments, the immunogenic composition comprises at least 45 μg of the mRNA construct(s). In certain embodiments, the immunogenic composition comprises 7.5 μg, 15 μg, 45 μg or 135 μg of the mRNA construct(s). Typically, reference to a certain μg amount of mRNA refers to the total dose of mRNA in the immunogenic composition. In certain embodiments, an immunogenic composition comprising an mRNA

construct of the invention, or a lipid nanoparticle encapsulating an mRNA construct of the invention, is for use in treating or preventing an infection caused by a β-coronavirus. In certain embodiments, the β-coronavirus expresses a spike protein which binds to angiotensin-converting enzyme 2 (ACE2). In a specific embodiment, the β-coronavirus is SARS-COV-2. In other embodiments, the β-coronavirus has a spike protein that is at least 75%, 80%, 90%, 95% or 99% identical to SEQ ID NO: 1.

The invention also provides a method of treating or preventing an infection caused by a β-coronavirus, said method comprising administering to a subject an effective amount of an immunogenic composition comprising an mRNA construct of the invention, or a lipid nanoparticle encapsulating an mRNA construct of the invention. In certain embodiments, the β-coronavirus expresses a spike protein which binds to angiotensin-converting enzyme 2 (ACE2). In a specific embodiment, the β-coronavirus is SARS-COV-2. In other embodiments, the β-coronavirus has a spike protein that is at least 75%, 80%, 90%, 95% or 99% identical to SEQ ID NO: 1. In particular embodiments, the immunogenic composition is administered to the subject at least twice. In certain embodiments, the period between administrations is at least 2 weeks. In some embodiments, the period between administrations is about 3 weeks.

In a particular embodiment, the invention provides an immunogenic composition comprising at least two nucleic acids, wherein the first nucleic acid comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline; and the second nucleic acid comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline and further contains the L18F, D80A, D215G, L242-, A243-, L244-, K417N, E484K, N501Y, D614G and A701V mutations.

In some embodiments, the first nucleic acid comprises an optimized nucleotide sequence which encodes the amino acid sequence of SEQ ID NO: 11. In particular embodiments, the first nucleic acid comprises an optimized nucleotide sequence that is at least 85% (e.g., at least 90%) identical to the nucleic acid sequence of SEQ ID NO: 44 or SEQ ID NO: 148 and encodes the amino acid sequence of SEQ ID NO: 11. In specific embodiments, the optimized nucleotide sequence has the nucleic acid sequence of SEQ ID NO: 44 or SEQ ID NO: 148.

In some embodiments, the second nucleic acid comprises an optimized nucleotide sequence which encodes the amino acid sequence of SEQ ID NO: 167. In particular embodiments, the second nucleic acid comprises an optimized nucleotide sequence that is at least 85% (e.g., at least 90%) identical to the nucleic acid sequence of SEQ ID NO: 168 or SEQ ID NO: 173 and encodes the amino acid sequence of SEQ ID NO: 167. In specific embodiments, the optimized nucleotide sequence has the nucleic acid sequence of SEQ ID NO: 166 or SEQ ID NO: 169.

In certain embodiments, the at least two nucleic acids are mRNA constructs. In specific embodiments, the optimized nucleotide sequence of the first nucleic acid has the nucleic acid sequence of SEQ ID NO: 148, and the optimized nucleotide sequence of the second nucleic acid has the nucleic acid sequence of SEQ ID NO: 173. In particular embodiments, the first nucleic acid is mRNA construct 1, and the second nucleic acid is mRNA construct 2. In certain embodiments, the at least two nucleic acids are encapsulated in lipid nanoparticles. In certain embodiments, the at least two nucleic acids are encapsulated in the same lipid nanoparticle. In certain embodiments, the at least two nucleic acids are encapsulated in separate lipid nanoparticles.

In some embodiments, the lipid nanoparticle comprises a cationic lipid, a non-cationic lipid, a cholesterol-based lipid and a PEG-modified lipid. In certain embodiments, the cationic lipid is selected from cKK-E12, cKK-E10, OF-Deg-Lin and OF-02; the non-cationic lipid is selected from DOPE and DEPE; the cholesterol-based lipid is cholesterol; and the PEG-modified lipid is DMG-PEG-2K. In specific embodiments, the lipid nanoparticle comprises cKK-E10, DOPE, cholesterol and DMG-PEG2K at the molar ratios 40:30:28.5:1.5. In further specific embodiments, the immunogenic composition comprises a total of 7.5 μg, 15 μg, 45 μg or 135 μg of the at least two nucleic acids.

The immunogenic composition described in paragraphs [0030]-[0034] can be used in the prophylaxis of an infection caused by a β-coronavirus. In certain embodiments, the β-coronavirus expresses a spike protein which binds to angiotensin-converting enzyme 2 (ACE2). In a specific embodiment, the β-coronavirus is SARS-COV-2. In other embodiments, the β-coronavirus has a spike protein that is at least 75% (e.g., at least 80%, 90%, 95% or 99%) identical to SEQ ID NO: 1.

In certain embodiments, the subject has not previously been administered an immunogenic composition for the prophylaxis of an infection caused by a β-coronavirus (e.g., SARS-COV-2), i.e., the immunogenic composition described in paragraphs [0030]-[0034] is the first immunogenic composition which is administered to the subject for that purpose. More commonly, the subject has previously been administered with one or more immunogenic composition(s) for the prophylaxis of an infection caused by a β-coronavirus (e.g., SARS-COV-2). For clarity “the subject has previously been administered with one or more immunogenic composition(s)” means that the subject has previously been administered with one or more doses of the same immunogenic composition or with one or more doses of different immunogenic composition(s)”. For example, the subject may have previously been administered with two immunogenic compositions at least two weeks apart for the prophylaxis of an infection caused by a β-coronavirus (e.g., SARS-COV-2). In some embodiments, these one or more immunogenic composition(s) is/are different from the immunogenic composition described in paragraphs [0030]-[0034]. In specific embodiments, the one or more immunogenic composition(s) is/are selected from a pharmaceutical compositions disclosed herein (e.g., an immunogenic composition or a vaccine disclosed herein) and a COVID-19 vaccine produced by Moderna (COVID-19 Vaccine Moderna, such as for example, mRNA-1273 or mRNA-1283), CureVac (CVnCOV), Johnson & Johnson (COVID-19 Vaccine Janssen), AstraZeneca (Vaxzevria), Pfizer/BioNTech (Comirnaty), Sputnik (Gam-COVID-Vac), Sinovac (COVID-19 Vaccine (Vero Cell) Inactivated) or Novavax (NVX-CoV2373). In certain embodiments, the immunogenic composition described in paragraphs [0030]-[0034] is administered 3-18 months after administration of the one or more immunogenic composition(s), which were previously administered to the subject for the prophylaxis of an infection caused by a β-coronavirus (e.g., SARS-COV-2). In certain embodiments, the immunogenic composition described in paragraphs [0030]-[0034] is administered at least 9 months or at least 12 months after administration of the one or more immunogenic composition(s), which were previously administered to the subject for the prophylaxis of an infection caused by a β-coronavirus (e.g., SARS-COV-2). In certain embodiments, the immunogenic composition described in paragraphs [0030]-[0034] is administered at least once, e.g., at least twice.

In another particular embodiment, the invention provides a method of treating or preventing an infection caused by a β-coronavirus, said method comprising administering to a subject an effective amount of an immunogenic composition comprising an mRNA construct, wherein said mRNA construct comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline and further contains the L18F, D80A, D215G, L242-, A243-, L244-, K417N, E484K, N501Y, D614G and A701V mutations. In some embodiments, the optimized nucleotide sequence encodes the amino acid sequence of SEQ ID NO: 167. In particular embodiments, the optimized nucleotide sequence comprises a nucleotide sequence that is at least 85% (e.g., at least 90%) identical to the nucleic acid sequence of SEQ ID NO: 166 or SEQ ID NO: 173 and encodes the amino acid sequence of SEQ ID NO: 167. In a specific embodiment, the optimized nucleotide sequence has the nucleic acid sequence of SEQ ID NO: 173. In certain embodiments, the mRNA construct is mRNA construct 2. In certain embodiments, the mRNA construct is encapsulated in a lipid nanoparticle. In certain embodiments, the lipid nanoparticle comprises a cationic lipid, a non-cationic lipid, a cholesterol-based lipid and a PEG-modified lipid. In certain embodiments, the cationic lipid is selected from cKK-E12, cKK-E10, OF-Deg-Lin and OF-02; the non-cationic lipid is selected from DOPE and DEPE; the cholesterol-based lipid is cholesterol; and the PEG-modified lipid is DMG-PEG-2K. In specific embodiments, the lipid nanoparticle comprises cKK-E10, DOPE, cholesterol and DMG-PEG2K at the molar ratios 40:30:28.5:1.5. In certain embodiments, the immunogenic composition comprises 7.5 μg, 15 μg, 45 μg or 135 μg of the mRNA construct. In certain embodiments, the β-coronavirus expresses a spike protein which binds to angiotensin-converting enzyme 2 (ACE2). In a specific embodiment, the β-coronavirus is SARS-COV-2. In other embodiments, the β-coronavirus has a spike protein that is at least 75%, 80%, 90%, 95% or 99% identical to SEQ ID NO:1.

In the method described in paragraph [0037], the subject may have not previously been administered an immunogenic composition for the prophylaxis of an infection caused by a β-coronavirus (e.g., SARS-COV-2). More commonly, the subject has previously been administered with one or more immunogenic composition(s) for the prophylaxis of an infection caused by a β-coronavirus (e.g., SARS-COV-2), e.g., two immunogenic compositions at least two weeks apart. In certain embodiments, the one or more immunogenic composition(s) is/are different from the immunogenic compositions of the invention. In certain embodiments, the one or more immunogenic composition(s) which has/have previously been administered to the subject is/are selected from a pharmaceutical compositions disclosed herein (e.g., an immunogenic composition or a vaccine disclosed herein) and a COVID-19 vaccine produced by Moderna (COVID-19 Vaccine Moderna, such as for example, mRNA-1273 or mRNA-1283), CureVac (CVnCOV), Johnson & Johnson (COVID-19 Vaccine Janssen), AstraZeneca (Vaxzevria), Pfizer/BioNTech (Comirnaty), Sputnik (Gam-COVID-Vac), Sinovac (COVID-19 Vaccine (Vero Cell) Inactivated) or Novavax (NVX-CoV2373). In certain embodiments, the method described in paragraph comprises administering the immunogenic composition described in that paragraph about 3-18 months after administration of the one or more immunogenic composition(s) which has/have previously been administered to the subject. In certain embodiments, the method described in paragraph comprises administering the immunogenic composition described in that paragraph [0037] at least 9 months or at least 12 months after administration of the one or more immunogenic composition(s). In certain embodiments, the method described in paragraph comprises administering the immunogenic composition described in that paragraph at least once, e.g., at least twice.

### BRIEF DESCRIPTION OF THE DRAWINGS

FIGS. 1A and 1B illustrate a process for generating optimized nucleotide sequences in accordance with the invention. As illustrated in FIG. 1A, the process receives an amino acid sequence of interest and a first codon usage table which reflects the frequency of each codon in a given organism (e.g., a mammal or human). The process then removes codons from the first codon usage table if they are associated with a codon usage frequency which is less than a threshold frequency (e.g., 10%). The codon usage frequencies of the codons not removed in the first step are normalized to generate a normalized codon usage table. The process uses the normalized codon usage table to generate a list of optimized nucleotide sequences. Each of the optimized nucleotide sequences encode the amino acid sequence of interest. As illustrated in FIG. 1B, the list of optimized nucleotide sequences is further processed by applying a motif screen filter, guanine-cytosine (GC) content analysis filter, and codon adaptation index (CAI) analysis filter, in that order, to generate an updated list of optimized nucleotide sequences.

FIG. 2 illustrates an example bar chart depicting the yield of protein produced from various codon optimized nucleotide sequences, determined by an ELISA assay for EPO.

FIG. 3 illustrates the structure of the spike protein of SARS-COV-2. SS=signal sequence; NTD=N-terminal domain; RBD=receptor binding domain; FP=fusion peptide; HR1=heptad repeat-N; CH, central helix; CTD, connector domain; HR2, heptad repeat 2; TM, transmembrane domain; CT, cytoplasmic tail. S2′, S2′ protease cleavage site are denoted with arrows. The PP and GSAS mutations lead to a prefusion conformations of the spike protein. This image is based FIG. 1 in Wrapp et al (2020) Science 367, 6483, 1260-1263.

FIG. 4 illustrates the spike protein of SARS-COV-2 and variants thereof that may form part of the pharmaceutical compositions disclosed herein or may be encoded by the optimized nucleotides sequences disclosed herein, e.g., for use in the nucleic acid-based vaccines disclosed here. Domains and subunits, mutations to remove the furin cleavage site and replace residues 985, 986 and 987 with proline (P, PP, PPP and GSAS mutations) and the relevant SEQ ID NOs are indicated. The same abbreviations are used as in FIG. 3.

FIGS. 5-7 demonstrate the protein production of nucleic acid vector constructs expressing optimized nucleic sequences encoding a full length native SARS-COV-2 S protein (Construct A) and three stable prefusion conformations of a SARS-COV-2 S protein (Constructs B-D). Construct B encodes a variant SARS-COV-2 S protein that is modified relative to naturally occurring SARS-COV-2 spike protein to lack the furin cleavage site (and therefore is not cleaved to form the S1 and S2 subunits) and to contain prolines as residues 986 and 987 (thereby stabilizing the protein in its prefusion conformation). Construct C encodes a variant SARS-COV-2 S protein that is modified relative to naturally occurring SARS-COV-2 spike protein to contain prolines as residues 986 and 987 and Construct D encodes a variant SARS-COV-2 S protein that is modified relative to naturally occurring SARS-COV-2 spike protein to lack the furin cleavage site.

FIGS. 5-6 show that constructs A and B can produce a glycosylated mature protein (˜225 kDa band) and a pre-processed full length S protein (˜170-180 kDa band).

FIG. 5 also shows the presence of S1 and S2 subunit bands with Construct A, demonstrating that the native full length SARS-COV-2 S protein is processed correctly by the cells.

FIG. 7 demonstrates that all four constructs were able to produce full length S protein. S1 and S2 subunit bands were detected with Construct A and Construct C. Strong bands of fully glycosylated mature S protein were detected with Construct B and Construct D.

FIG. 8 illustrates the spike protein of SARS-COV-2 and variants thereof that may form part of the pharmaceutical compositions disclosed herein or may be encoded by the optimized nucleotides sequences disclosed herein, e.g., for use in the nucleic acid-based vaccines disclosed here. Domains, subunits, mutations to remove the furin cleavage site and mutate residues 817, 892, 899, 942, 986 and 987 with proline (P, PP, PPP, PPPPP and GSAS), the D614G mutation, removal of the ER retrieval signal and an extended N-terminal signal peptide and the relevant SEQ ID NOs are indicated. The same abbreviations are used as in FIG. 3.

FIG. 9 illustrates that an immunogenic composition of lipid nanoparticle (LNP)-encapsulated mRNA comprising an optimized nucleotide sequence encoding a full-length uncleavable pre-fusion stabilized SARS-COV-2 S protein produced a robust binding and neutralizing antibody response in mice. FIG. 9A illustrates the ELISA titers elicited in mice after immunization with two doses of 0.2 μg, 1 μg, 5 μg or 10 μg LNP-encapsulated mRNA. A group of mice to which the diluent of the mRNA-LNP composition was administered acted as a negative control. FIG. 9B illustrates the titer of neutralizing antibodies produced in mice after immunization with two doses of either 0.2 μg, 1 μg, 5 μg or 10 μg LNP-encapsulated mRNA as determined by a pseudovirus-based assay. 39 individual conversion serum samples from COVID-19 patients with mild, strong and severe symptoms (Conv Sera) acted as a positive control. As illustrated in FIG. 9C, the immunogenic composition was administered on Day 0 and Day 21. Blood was sampled on days Day −7 (baseline), Day 14, Day 21, Day 28 and Day 35.

FIG. 10 illustrates that an immunogenic composition of LNP-encapsulated mRNA comprising an optimized nucleotide sequence encoding a full-length uncleavable pre-fusion stabilized SARS-COV-2 S protein produced a Th1-biased T-cell response in mice. FIG. 10A shows that splenocytes isolated at Day 35 secreted high levels of the Th1 cytokine interferon-γ (IFN-γ). FIG. 10B shows that these splenocytes did not secrete detectable amounts of the Th2 cytokine IL-5. As illustrated in FIG. 10C, the mice were immunized with two doses of either 5 μg or 10 μg LNP-encapsulated mRNA at Day 0 and Day 21, blood was sampled on days Day −4, Day 14, Day 21, Day 28 and Day 35, and spleens were harvested at Day 35 for determination of IFN-γ and IL-5 levels by ELISPOT assay.

FIG. 11 illustrates that an immunogenic composition of LNP-encapsulated mRNA comprising an optimized nucleotide sequence encoding a full-length uncleavable pre-fusion stabilized SARS-COV-2 S protein produced a robust binding and neutralizing antibody response in cynomolgus monkeys. FIG. 11A illustrates the ELISA titer elicited in cynomolgus monkeys after immunization with two doses of 15 μg, 45 μg or 135 μg LNP-encapsulated mRNA. FIG. 11B illustrates the titers of neutralizing antibodies produced in cynomolgus monkeys after immunization with two doses of 15 μg, 45 μg or 135 μg LNP-encapsulated mRNA, as determined by a pseudovirus-based assay. FIG. 11C illustrates the microneutralization titers produced in cynomolgus monkeys after immunization with two doses of either 15 μg, 45 μg or 135 μg LNP-encapsulated mRNA. 39 individual conversion serum samples from COVID-19 patients with mild, strong and severe symptoms (Conv Sera) acted as a positive control in the assays illustrated by FIGS. 11B and 11C. As illustrated in FIG. 11D, the immunogenic composition was administered on Day 0 and Day 21. Blood was sampled on days Day −4 (baseline), Day 2, Day 7, Day 14, Day 21, Day 23, Day 28 and Day 35 and Day 42. Peripheral blood mononuclear cells (PMCs) were isolated on Day 42 to determine the cell-mediated immunity (CMI) elicited by the test composition.

FIG. 12 illustrates that an immunogenic composition of LNP-encapsulated mRNA comprising an optimized nucleotide sequence encoding a full-length uncleavable pre-fusion stabilized SARS-COV-2 S protein produced a Th1-biased T-cell response in cynomolgus monkeys. The monkeys were immunized with two doses of either 5 μg or 10 μg LNP-encapsulated mRNA at Day 0 and Day 21. FIGS. 12A and 12C show that PMBCs isolated on Day 42 secreted high levels of the Th1 cytokine interferon-γ (IFN-γ) after stimulation with peptide pools S1 and S2, respectively (SARS-COV-2 S protein-derived peptides). FIGS. 12B and 12D show that these PMBCs secreted only baseline levels of the Th2 cytokine IL-13 in response to peptide stimulation. Naïve (non-activated and non-stimulated) splenocytes served as a control to establish baseline levels of IFN-γ and IL-13 (dashed line).

FIG. 13 describes a statistical analysis of the data summarized in FIGS. 9 and 11. Pseudovirus (PsV) titers in mice for the 1 μg, 5 μg and 10 μg dose levels of the tested immunogenic composition were significantly different from the control human convalescent sera PsV titers (FIG. 13A). Spearman Correlation Coefficients (SCC) between ELISA (IgG), pseudoviral (PsV) and microneutralization (MN) titers were calculated for the cynomolgus monkey experiment summarised in FIG. 11. SCC were conducted per individual animals, and means (±Standard Errors) were calculated per dose (N=4) or all test animals (N=12). The results of this analysis are shown in FIG. 13B. FIGS. 13C and 13D illustrate that microneutralization (MN) and pseudoviral (PsV) titers in cynomolgus monkeys were significantly higher than MN and PsV titers of human convalescent sera that served as controls.

FIG. 14 illustrates the neutralizing antibody titers induced in mice and NHPs by immunization with LNP formulations comprising optimized mRNAs encoding full-length prefusion stabilized SARS-COV-2 S proteins. Mice were administered two immunizations at a three-week interval with a 0.4 μg per dose of each of five formulations (WT, 2P, GSAS, 2P/GSAS, 2P/GSAS/ALAYT). Non-human primates (NHPs) were immunized using the same immunization schedule at 5 μg per dose of six formulations (2P, GSAS, 2P/GSAS, 2P/GSAS/ALAYT, 6P and 6P/GSAS). Sera samples were collected from pre-immunized animals (Day −4) and on Day 14, 21, 28, 35 and 42 post administration. Each dot represents an individual serum sample and the line represents the geometric mean for the group. The dotted line below for each panel represents the lower limit of assay readout.

FIG. 15 illustrates the protective efficacy of LNP formulation of Example 5 in Syrian golden hamsters. (a) weight loss in hamsters administered with either a single or two dose regime; (b) H&E staining of lungs of hamsters that received either one dose 0.15 μg ( [symbol] ), 1.5 μg ( [symbol] ), 4.5 μg ( [symbol] ), 13.5 μg ( [symbol] ), Sham ( [symbol] ) or unchallenged (-∘-) animals; (c) Day 4 and Day 7 post-challenge pathogenicity scores of hamsters immunized with either one or two dose regimens; (d) Quantification of SARS-COV-2 subgenomic mRNA (sgmRNA) in lungs and nasal tissue of hamsters immunized with two doses of the LNP formulation of Example 5 as compared to control (Sham and Naïve) on Day 4 and Day 7 post-infection (DPI).

FIG. 16 provides the strains from which the S protein was derived for the preparation of pseudoviruses (PsVs) that were used in the neutralization assays described in Example 14. For SARS-COV-2 strains, mutations compared to the SARS-COV-2 S protein from the Wuhan index strain are indicted as well as the presence of the D614G mutation. Where applicable, the GenBank number of the S-protein amino acid sequence is provided. The PsVs were obtained from Integral Molecular, and both the catalogue number and the lot number for each PsV are also indicated.

FIG. 17 illustrates that non-human primates (NHPs), which previously had been immunized with two doses of the LNP formulation of Example 5, mount an effective neutralizing antibody response against the S protein derived from the original Wuhan strain as well as naturally occurring variants of the S protein observed in South Africa, Japan/Brazil and California, and an S protein derived from a SARS-COV-1 strain after immunization with a booster mRNA vaccine encoding a South African variant of the SARS-COV-2 S protein. NHPs were administered two immunizations on day 0 and day 35 with LNP formulations that comprised an optimized mRNAs encoding full-length prefusion stabilized SARS-COV-2 S protein as described in Example 5. A booster LNP formulation comprising an mRNA encoding a corresponding S protein with mutations observed in a naturally occurring South African strain was injected on Day 305. Serum samples were taken on days 35, 308, 329 and 343. Each dot represents an individual serum sample, and the line represents the geometric mean for the group. The dotted line represents the lower limit of detection.

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The terms “e.g.,” and “i.e.” as used herein, are used merely by way of example, without limitation intended, and should not be construed as referring only those items explicitly enumerated in the specification.

Unless specifically stated or evident from context, as used herein, the term “about” is understood as within a range of normal tolerance in the art, for example within 2 standard deviations of the mean. “About” can be understood to be within 10%, 9%, 8%, 7%, 6%, 5%, 4%, 3%, 2%, 1%, 0.5%, 0.1%, 0.05%, 0.01%, or 0.001% of the stated value. Unless otherwise clear from the context, all numerical values provided herein reflects normal fluctuations that can be appreciated by a skilled artisan.

As used herein, term “abortive transcript” or “pre-aborted transcript” or the like is any transcript that is shorter than a full-length mRNA molecule encoded by the DNA template that results from the premature release of RNA polymerase from the template DNA in a sequence-independent manner. In some embodiments, an abortive transcript may be less than 90% of the length of the full-length mRNA molecule that is transcribed from the target DNA molecule, e.g., less than 80%, 70%, 60%, 50%, 40%, 30%, 20%, 10%, 5%, 1% of the length of the full-length mRNA molecule.

As used herein, the terms “codon” and “codons” refer to a sequence of three nucleotides which together form a unit of the genetic code. Each codon corresponds to a specific amino acid or stop signal in the process of translation or protein synthesis. The genetic code is degenerate, and more than one codon can encode a specific amino acid residue. For example, codons can comprise DNA or RNA nucleotides.

As used herein, the terms “codon optimization” and “codon-optimized” refer to modifications of the codon composition of a naturally-occurring or wild-type nucleic acid encoding a peptide, polypeptide or protein that do not alter its amino acid sequence, thereby improving protein expression of said nucleic acid. In the context of the present invention, “codon optimization” may also refer to the process by which one or more optimized nucleotide sequences are arrived at by removing with filters less than optimal nucleotide sequences from a list of nucleotide sequences, such as filtering by guanine-cytosine content, codon adaptation index, presence of destabilizing nucleic acid sequences or motifs, and/or presence of pause sites and/or terminator signals.

As used herein, “full-length mRNA” is as characterized when using a specific assay, e.g., gel electrophoresis and detection using UV and UV absorption spectroscopy with separation by capillary electrophoresis. The length of an mRNA molecule that encodes a full-length polypeptide is at least 50% of the length of a full-length mRNA molecule that is transcribed from the target DNA, e.g., at least 60%, 70%, 80%, 90%, 91%, 92%, 93%, 94%, 95%, 96%, 97%, 98%, 99%, 99.01%, 99.05%, 99.1%, 99.2%, 99.3%, 99.4%, 99.5%, 99.6%, 99.7%, 99.8%, 99.9% of the length of a full-length mRNA molecule that is transcribed from the target DNA.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, the term “messenger RNA (mRNA)” refers to a polyribonucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, in vitro transcribed, or chemically synthesized. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. A nucleic acid sequence is presented in the 5′ to 3′ direction unless otherwise indicated.

As used herein, the term “nucleotide sequence”, in its broadest sense, refers to the order of nucleobases within a nucleic acid. In some embodiments, “nucleotide sequence” refers to the order of individual nucleobases within a gene. In some embodiments, “nucleotide sequence” refers to the order of individual nucleobases within a protein-coding gene. In some embodiments, “nucleotide sequence” refers to the order of individual nucleobases within single and/or double stranded DNA and/or cDNA. In some embodiments, “nucleotide sequence” refers to the order of individual nucleobases within RNA. In some embodiments, “nucleotide sequence” refers to the order of individual nucleobases within mRNA. In a particular embodiment, “nucleotide sequence” refers to the order of individual nucleobases within the protein-coding sequence of RNA or DNA. A nucleotide sequence is normally presented in the 5′ to 3′ direction unless otherwise indicated.

As used herein, the term “premature termination” refers to the termination of transcription before the full length of the DNA template has been transcribed. As used herein, premature termination can be caused by the presence of a nucleotide sequence motif (also referred to herein simply as “motif”), e.g., a termination signal, within the DNA template and results in mRNA transcripts that are shorter than the full length mRNA (“prematurely terminated transcripts” or “truncated mRNA transcripts”). Examples of a termination signal include the E. coli rrnB terminator t1 signal (consensus sequence: ATCTGTT) and variants thereof, as described herein.

As used herein, the term “template DNA” (or “DNA template”) relates to a DNA molecule comprising a nucleic acid sequence encoding an mRNA transcript to be synthesized by in vitro transcription. The template DNA is used as template for in vitro transcription in order to produce the mRNA transcript encoded by the template DNA. The template DNA comprises all elements necessary for in vitro transcription, particularly a promoter element for binding of a DNA-dependent RNA polymerase, such as, e.g., T3, T7 and SP6 RNA polymerases, which is operably linked to the DNA sequence encoding a desired mRNA transcript. Furthermore the template DNA may comprise primer binding sites 5′ and/or 3′ of the DNA sequence encoding the mRNA transcript to determine the identity of the DNA sequence encoding the mRNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleic acid sequence encoding the desired mRNA transcript.

As used herein, the term “preventing” refers to partially or completely inhibiting the onset of one or more symptoms or features of a particular infection, disease, disorder, and/or condition.

As used herein, the term “prophylaxis” refers to partially or completely inhibiting the onset of one or more symptoms or features of a particular infection, disease, disorder, and/or condition.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition.

As used herein, the term “immunogenic composition” means a composition comprising a nucleic acid or protein that, when administered to a subject, elicits an immune response. In some embodiments, the “immunogenic composition” comprises a nucleic acid. In some embodiments, the nucleic acid is mRNA. In some embodiments, the nucleic acid is DNA. It should be understood that the terms “immunogenic composition” and “vaccine” are used interchangeably herein and are thus meant to have equivalent meanings.

Percentage sequence identity between two nucleotide (or amino acid) sequences is determined after alignment of the two sequences. This alignment and the percentage sequence identity can be determined using software programs known in the art, for example those described in section 7.7.18 of Current Protocols in Molecular Biology (F. M. Ausubel et al., eds., 1987) Supplement 30. In the context of the present invention, an alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2:482-489. A comparison is then carried out between respective nucleotides (or amino acids) located at the same position in the two nucleotide (or amino acid) sequences. When a given position is occupied by the same nucleotide (or amino acid) in the two nucleotide (or amino acid) sequences, these sequences are identical for this position. The percentage of sequence identity is then determined from the number of positions for which respective nucleotides (or amino acids) are identical, over the total number of nucleotides (or amino acids) in the nucleotide (or amino acid) sequence with which the comparison is made.

All technical and scientific terms used herein have the same meaning as commonly understood by one of ordinary skill in the art to which this application belongs and as commonly used in the art to which this application belongs. The publications and other reference materials referenced herein to describe the background of the invention and to provide additional detail regarding its practice are hereby incorporated by reference.

### DETAILED DESCRIPTION OF THE INVENTION

The present invention addresses the need for generating optimized nucleotide sequences encoding a protein antigen for the effective treatment or prevention of an infectious disease through the provision of a vaccine comprising an mRNA with the optimized nucleotide sequence. A method is provided for processing a naturally occurring nucleotide sequence encoding a protein antigen to produce at least one optimized nucleotide sequence. The optimized nucleotide sequence is designed to increase the expression of the encoded protein antigen compared to the expression of the protein associated with the naturally occurring nucleotide sequence. Codon optimization can modify the composition of a protein-coding nucleotide sequence based on various criteria without altering the sequence of translated amino acids of the encoded protein antigen, due to the redundancy in the genetic code.

To avoid imbalance between mRNA codon usage and abundance of cognate tRNAs, codon optimization can provide a composition of codons within a nucleotide sequence that better matches the naturally occurring abundance of transfer RNAs (tRNAs) in a host cell and avoids depletion of a specific tRNA. As tRNA abundance influences the rate of protein translation, codon optimization of a nucleotide sequence can increase the efficiency of protein translation and yield for the encoded protein. For example, by not using rare codons which are characterized by a low codon usage, efficiency of protein translation and protein yield can be increased, as the shortage of rare tRNAs can stall or terminate protein translation.

Codon optimization can come at the cost of reduced functional activity of the encoded protein and an associated loss in efficacy as the process may remove information encoded in the nucleotide sequence that is important for controlling translation of the protein and ensuring proper folding of the nascent polypeptide chain (Mauro & Chappell, Trends Mol Med. 2014; 20 (11): 604-13). The inventors have found that optimized sequences which retain some variety, i.e. do not necessarily include only one codon encoding each amino acid, can achieve increased protein yield while retaining functional activity of the encoded protein.

### Generation of Optimized Nucleotide Sequences

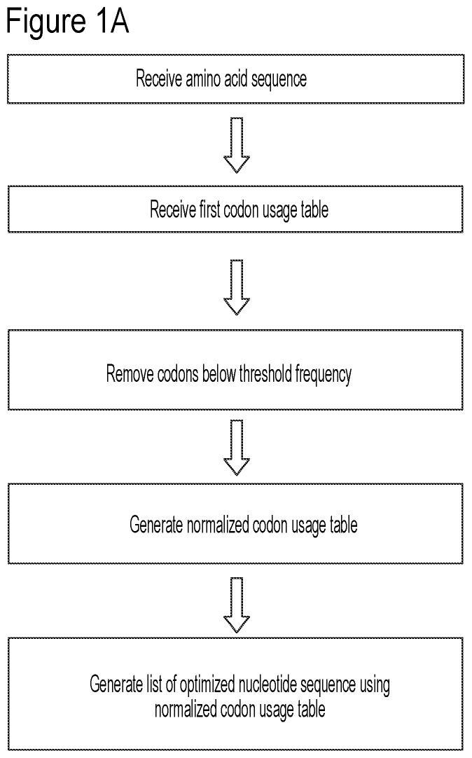

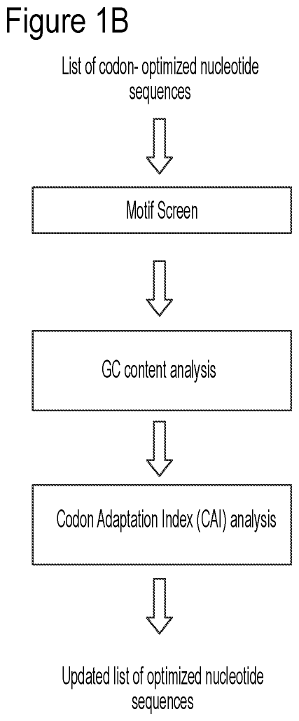

FIGS. 1A and 1B illustrate a process for generating optimized nucleotide sequences in accordance with the invention. The process first generates a list of codon-optimized sequences and then applies three filters to the list. Specifically, it applies a motif screen filter, guanine-cytosine (GC) content analysis filter, and codon adaptation index (CAI) analysis filter to produce an updated list of optimized nucleotide sequences. The updated list no longer includes nucleotide sequences containing features that are expected to interfere with effective transcription and/or translation of the encoded protein antigen.

### Codon Optimization

The genetic code has 64 possible codons. Each codon comprises a sequence of three nucleotides. The usage frequency for each codon in the protein-coding regions of the genome can be calculated by determining the number of instances that a specific codon appears within the protein-coding regions of the genome, and subsequently dividing the obtained value by the total number of codons that encode the same amino acid within protein-coding regions of the genome. A codon usage table contains experimentally derived data regarding how often, for the particular biological source from which the table has been generated, each codon is used to encode a certain amino acid. This information is expressed, for each codon, as a percentage (0 to 100%), or fraction (0 to 1), of how often that codon is used to encode a certain amino acid relative to the total number of times a codon encodes that amino acid.

Codon usage tables are stored in publically available databases, such as the Codon Usage Database (Nakamura et al. (2000) Nucleic Acids Research 28 (1), 292; available online at https://www.kazusa.or.jp/codon/), and the High-performance Integrated Virtual Environment-Codon Usage Tables (HIVE-CUTs) database (Athey et al., (2017), BMC Bioinformatics 18 (1), 391; available online at http://hive.biochemistry.gwu.edu/review/codon).

During the first step of codon optimization, codons are removed from a first codon usage table which reflects the frequency of each codon in a given organism (e.g., a mammal or human) if they are associated with a codon usage frequency which is less than a threshold frequency (e.g., 10%). The codon usage frequencies of the codons not removed in the first step are normalized to generate a normalized codon usage table. An optimized nucleotide sequence encoding an amino acid sequence of interest is generated by selecting a codon for each amino acid in the amino acid sequence based on the usage frequency of the one or more codons associated with a given amino acid in the normalized codon usage table. The probability of selecting a certain codon for a given amino acid is equal to the usage frequency associated with the codon associated with this amino acid in the normalized codon usage table.

The codon-optimized sequences of the invention are generated by a computer-implemented method for generating an optimized nucleotide sequence. The method comprises: (i) receiving an amino acid sequence, wherein the amino acid sequence encodes a peptide, polypeptide, or protein; (ii) receiving a first codon usage table, wherein the first codon usage table comprises a list of amino acids, wherein each amino acid in the table is associated with at least one codon and each codon is associated with a usage frequency; (iii) removing from the codon usage table any codons associated with a usage frequency which is less than a threshold frequency; (iv) generating a normalized codon usage table by normalizing the usage frequencies of the codons not removed in step (iii); and (v) generating an optimized nucleotide sequence encoding the amino acid sequence by selecting a codon for each amino acid in the amino acid sequence based on the usage frequency of the one or more codons associated with the amino acid in the normalized codon usage table. The threshold frequency can be in the range of 5%-30%, in particular 5%, 10%, 15%, 20%, 25%, or 30%. In the context of the present invention, the threshold frequency is typically 10%.

The step of generating a normalized codon usage table comprises: (a) distributing the usage frequency of each codon associated with a first amino acid and removed in step (iii) to the remaining codons associated with the first amino acid; and (b) repeating step (a) for each amino acid to produce a normalized codon usage table. In some embodiments, the usage frequency of the removed codons is distributed equally amongst the remaining codons. In some embodiments, the usage frequency of the removed codons is distributed amongst the remaining codons proportionally based on the usage frequency of each remaining codon. “Distributed” in this context may be defined as taking the combined magnitude of the usage frequencies of removed codons associated with a certain amino acid and apportioning some of this combined frequency to each of the remaining codons encoding the certain amino acid.

The step of selecting a codon for each amino acid comprises: (a) identifying, in the normalized codon usage table, the one or more codons associated with a first amino acid of the amino acid sequence; (b) selecting a codon associated with the first amino acid, wherein the probability of selecting a certain codon is equal to the usage frequency associated with the codon associated with the first amino acid in the normalized codon usage table; and (c) repeating steps (a) and (b) until a codon has been selected for each amino acid in the amino acid sequence.

The step of generating an optimized nucleotide sequence by selecting a codon for each amino acid in the amino acid sequence (step (v) in the above method) is performed n times to generate a list of optimized nucleotide sequences.

### Motif Screen

A motif screen filter is applied to the list of optimized nucleotide sequences. Optimized nucleotide sequences encoding any known negative cis-regulatory elements and negative repeat elements are removed from the list to generate an updated list.

For each optimized nucleotide sequence in the list, it is also determined whether it contains a termination signal. Any nucleotide sequence that contains one or more termination signals is removed from the list generating an updated list. In some embodiments, the termination signal has the following nucleotide sequence: 5′-X₁ATCTX₂TX₃-3′, wherein X₁, X₂ and X₃ are independently selected from A, C, T or G. In some embodiments, the termination signal has one of the following nucleotide sequences: TATCTGTT; and/or TTTTTT; and/or AAGCTT; and/or GAAGAGC; and/or TCTAGA. In some embodiments, the termination signal has the following nucleotide sequence: 5′-X₁AUCUX₂UX₃-3′, wherein X₁, X₂ and X₃ are independently selected from A, C, U or G. In some embodiments, the termination signal has one of the following nucleotide sequences: UAUCUGUU; and/or UUUUUU; and/or AAGCUU; and/or GAAGAGC; and/or UCUAGA.

### Guanine-Cytosine (GC) Content

The method further comprises determining a guanine-cytosine (GC) content of each of the optimized nucleotide sequences in the updated list of optimized nucleotide sequences. The GC content of a sequence is the percentage of bases in the nucleotide sequence that are guanine or cytosine. The list of optimized nucleotide sequences is further updated by removing any nucleotide sequence from the list, if its GC content falls outside a predetermined GC content range.

Determining a GC content of each of the optimized nucleotide sequences comprises, for each nucleotide sequence: determining a GC content of one or more additional portions of the nucleotide sequence, wherein the additional portions are non-overlapping with each other and with the first portion, and wherein updating the list of optimized sequences comprises: removing the nucleotide sequence if the GC content of any portion falls outside the predetermined GC content range, optionally wherein determining the GC content of the nucleotide sequence is halted when the GC content of any portion is determined to be outside the predetermined GC content range. In some embodiments, the first portion and/or the one or more additional portions of the nucleotide sequence comprise a predetermined number of nucleotides, optionally wherein the predetermined number of nucleotides is in the range of: 5 to 300 nucleotides, or 10 to 200 nucleotides, or 15 to 100 nucleotides, or 20 to 50 nucleotides. In the context of the present invention, the predetermined number of nucleotides is typically 30 nucleotides. The predetermined GC content range can be 15%-75%, or 40%-60%, or, 30%-70%. In the context of the present invention, the predetermined GC content range is typically 30%-70%.

A suitable GC content filter in the context of the invention may first analyze the first 30 nucleotides of the optimized nucleotide sequence, i.e., nucleotides 1 to 30 of the optimized nucleotide sequence. Analysis may comprise determining the number of nucleotides in the portion with are either G or C, and determining the GC content of the portion may comprise dividing the number of G or C nucleotides in the portion by the total number of nucleotides in the portion. The result of this analysis will provide a value describing the proportion of nucleotides in the portion that are G or C, and may be a percentage, for example 50%, or a decimal, for example 0.5. If the GC content of the first portion falls outside a predetermined GC content range, the optimized nucleotide sequence may be removed from the list of optimized nucleotide sequences.

If the GC content of the first portion falls inside the predetermined GC content range, the GC content filter may then analyze a second portion of the optimized nucleotide sequence. In this example, this may be the second 30 nucleotides, i.e., nucleotides 31 to 60, of the optimized nucleotide sequence. The portion analysis may be repeated for each portion until either: a portion is found having a GC content falling outside the predetermined GC content range, in which case the optimized nucleotide sequence may be removed from the list, or the whole optimized nucleotide sequence has been analyzed and no such portion has been found, in which case the GC content filter retains the optimized nucleotide sequence in the list and may move on to the next optimized nucleotide sequence in the list.

### Codon Adaptation Index (CAI)

The method further comprises determining a codon adaptation index of each of the optimized nucleotide sequences in the most recently updated list of optimized nucleotide sequences. The codon adaptation index of a sequence is a measure of codon usage bias and can be a value between 0 and 1. The most recently updated list of optimized nucleotide sequences is further updated by removing any nucleotide sequence if its codon adaptation index is less than or equal to a predetermined codon adaptation index threshold. The codon adaptation index threshold can 0.7, or 0.75, or 0.8, or 0.85, or 0.9. The inventors have found that optimized nucleotide sequences with a codon adaptation index equal to or greater than 0.8 deliver very high protein yield. Therefore in the context of the invention, the codon adaptation index threshold is typically 0.8.

A codon adaptation index may be calculated, for each optimized nucleotide sequence, in any way that would be apparent to a person skilled in the art, for example as described in “The codon adaptation index—a measure of directional synonymous codon usage bias, and its potential applications” (Sharp and Li, 1987. Nucleic Acids Research 15 (3), p. 1281-1295); available online at https://www.ncbi.nlm.nih.gov/pmc/articles/PMC340524/.

Implementing a codon adaptation index calculation may include a method according to, or similar to, the following. For each amino acid in a sequence, a weight of each codon in a sequence may be represented by a parameter termed relative adaptiveness (wᵢ). Relative adaptiveness may be computed from a reference sequence set, as the ratio between the observed frequency of the codon fᵢ and the frequency of the most frequent synonymous codon fⱼ for that amino acid. The codon adaptation index of a sequence may then be calculated as the geometric mean of the weight associated to each codon over the length of the sequence (measured in codons). The reference sequence set used to calculate codon adaptation index may be the same reference sequence set from which a codon usage table used with methods of the invention is derived.

### Synthesis of Optimized Nucleotide Sequences

Once a list of optimized nucleotide sequences has been generated, in vitro synthesis (also referred to commonly as “in vitro transcription”) can be performed with a nucleic acid vector such as a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNase I, pyrophosphatase, and/or RNase inhibitor. The exact conditions will vary according to the specific application.

The nucleic acid vector typically is a plasmid. The term ‘plasmid’ or ‘plasmid nucleic acid vector’ refers to a circular nucleic acid molecule, e.g., to an artificial nucleic acid molecule. A plasmid DNA in the context of the present invention is suitable for incorporating or harboring a desired nucleic acid sequence, such as a nucleic acid sequence comprising a sequence encoding an mRNA transcript and/or an open reading frame encoding at least protein antigen. Such plasmid DNA constructs/vectors may be expression vectors, cloning vectors, transfer vectors etc.

The nucleic acid vector typically comprises a sequence corresponding to (coding for) a desired mRNA transcript, or a part thereof, such as a sequence corresponding to the optimized nucleotide sequence encoding a protein antigen and the 5′- and/or 3′UTR of an mRNA. In some embodiments, the sequence corresponding to the desired mRNA transcript may also encode a poly A-tail after the 3′ UTR so that the poly A-tail is included with the mRNA transcript. More typically in the context of the present invention, the sequence corresponding to the desired mRNA transcript consists of the 5′/3′ UTRs and the open reading frame. In some embodiments of the invention, the mRNA transcript synthesized from the nucleic acid vector during in vitro transcription does not contain a polyA tail. A polyA tail may be added to the mRNA transcript in a post-synthesis processing step.

### Screening of Optimized Nucleotide Sequences

Individual in vitro transcribed, capped and tailed mRNAs encoding an optimized nucleotide sequence encoding a protein antigen can be transfected into a cell either in vivo or in vitro to determine the expression level of the protein encoded by the optimized nucleotide sequence. An mRNA encoding, e.g., a naturally occurring nucleotide sequence encoding the protein antigen, or a codon-optimized nucleotide sequence encoding the protein antigen prepared with a method other than the process for generating an optimized nucleotide sequence described herein, may serve as a control mRNA. Each mRNA and control mRNA are contacted with a separate cell or organism, wherein the cell or organism contacted. An mRNA comprising an optimized nucleotide sequence generated in accordance with the invention is selected for use in a immunogenic composition in accordance with the present invention if it produces an increased yield of the protein antigen compared to the yield of the protein produced by the cell or organism contacted with a control mRNA.

Methods well-known in the art, such as western blotting, are suitable to experimentally verify that the optimized nucleotide sequence results in increased expression and production of the encoded protein antigen. Furthermore, multiple optimized nucleotide sequences generated by the methods of the present invention can be screened to identify the sequence or sequences which generate the highest protein yield. In some embodiments, the expression level of the protein encoded by the optimized nucleotide sequence is increased at least 2-fold, e.g., at least 3-fold or 4-fold.

In some embodiments, the functional activity of the protein antigen encoded by the optimized nucleotide sequence is determined. The functional activity of the protein encoded by the optimized nucleotide sequence can be determined using a range of well-established methods. These methods may vary depending on the properties of the encoded protein antigen. For example, antibodies recognizing a conformational epitope on the protein antigen may be used to confirm proper folding of the protein antigen expressed from the optimized nucleotide sequence. Alternatively or in addition, in embodiments of the invention relating to a spike protein of SARS-CoV-2, the spike protein may be contacted with human angiotensin-converting enzyme 2 (ACE2) to confirm its receptor binding activity. Binding activity is typically assessed relative to a control, such a spike protein of SARS-COV-2 expressed from a naturally occurring coding sequence.

### SARS-COV-2 Proteins

Coronaviruses (CoVs) are the largest group of viruses belonging to the Nidovirales order, which includes Coronaviridae, Arteriviridae, and Roniviridae families. CoVs are spherical enveloped viruses with a positive-sense single-stranded RNA genome and a nucleocapsid of helical symmetry with a diameter of approximately 125 nm.

SARS-COV-2 is a β-coronavirus, like other coronaviruses that infect humans, such as MERS-COV and SARS-COV. The first two-thirds of the viral 30 kb RNA genome, mainly named as ORF1a/b region, encodes two polyproteins (pp1a and pp1ab), which constitute the main non-structural proteins. The remaining genome encodes accessory proteins and four essential structural proteins, namely the spike(S) glycoprotein, small envelope (E) protein, matrix/membrane (M) protein, and nucleocapsid (N) proteins (Kang et al. (2020) https://doi.org/10.1101/2020.03.06.977876). SARS-COV-2 uses its S protein to bind host cell receptors (ACE2 in human) and mediate cell entry. This makes S protein the main target for neutralizing antibodies, as discussed in detail below.

### Spike Glycoprotein (S Protein)

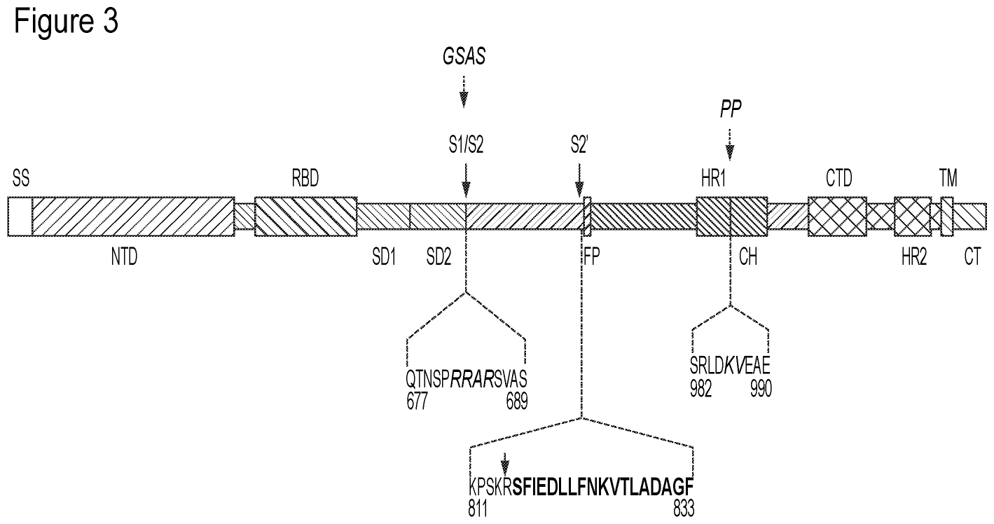

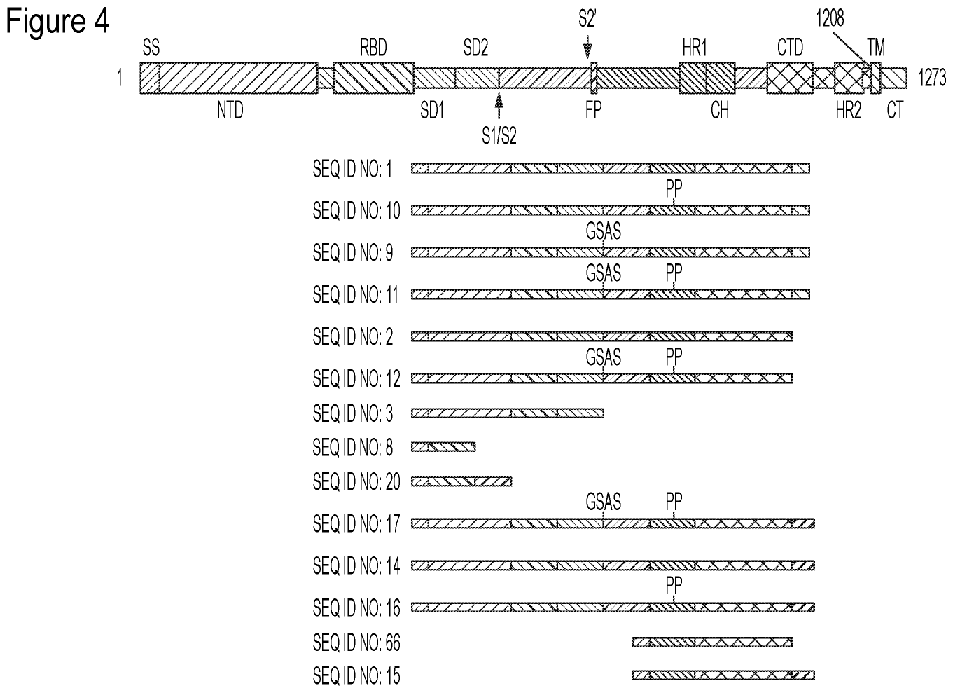

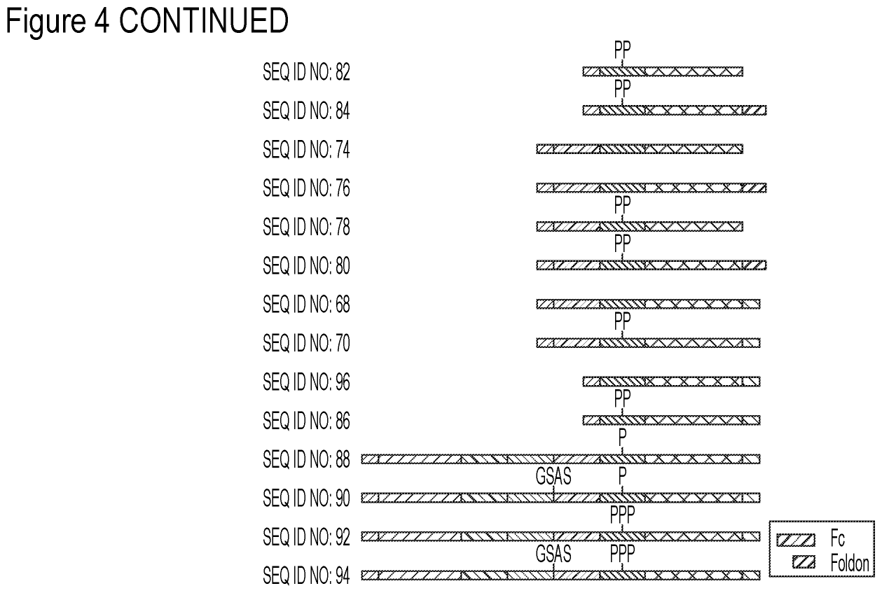

Cell entry depends on the binding of S proteins to receptors on the cell surface and on S protein priming by host cell proteases. The S protein comprises two functional subunits responsible for binding to the host cell receptor (S1 subunit) and fusion of the viral and cellular membranes (S2 subunit) (FIG. 3). The S protein forms a homotrimer that produces a distinctive spike structure on the surface of the virus. The S1 subunit has a large receptor-binding domain (RBD), while S2 forms the stalk of the spike molecule. The amino acid sequence of the full-length SARS-COV-2 S glycoprotein is provided by SEQ ID NO: 1 (Gen Bank QHD43416.1). The S1 subunit is located at residues 1 to 681, the S2 subunit is located at residues 686 to 1208 and the S2′ subunit is located at residues 816 to 1208. The C-terminal end of the S protein contains a transmembrane domain, and the last 19 amino acids of the cytoplasmic tail contain an endoplasmic reticulum (ER)-retention signal.

References to the naturally occurring SARS-COV-2 S protein refer to the full-length SARS-COV-2 S glycoprotein provided by SEQ ID NO: 1. Any modifications to the naturally occurring SARS-COV-2 S protein are numbered based on the residues in SEQ ID NO:1

Although the observed diversity among pandemic SARS-COV-2 sequences is low, its rapid global spread provides the virus with ample opportunity for natural selection to act upon rare but favorable mutations. It is advantageous to target the sequences of the circulating SARS-CoV-2 virus rather than just the index strain from Wuhan (i.e. SEQ ID NO: 1).

An amino acid change in the SARS-COV-2 S glycoprotein, D614G, emerged early during the 2020 COVID-19 pandemic and as of July 2020 has become the most prevalent form of the virus around the world. Patients infected with G614 shed more viral nucleic acid compared with those with D614, and G614-bearing viruses show significantly higher infectious titers in vitro than their D614 counterparts (Korber et al., 2020, Cell 182, 1-16). Optimized nucleotide sequence encoding a SARS-COV-2 S protein comprising a D614G mutation may therefore particularly suitable for use in immunogenic composition as described herein.

Other rare mutations that have been identified in the SARS-COV-2 S protein are summarized in the table below (Korber et al. 2020-https://doi.org/10.1101/2020.04.29.069054):

| Spike Mutation | Spike location possible impact |
|---|---|
| L5F | Signal Peptide |
| L8V/W | Signal Peptide |
| H49Y | S1 NTD domain |
| Y145H/del | S1 NTD domain |
| Q239K | S1 NTD domain |
| V367F | Up/Down conformations |
| G476S | Directly in the RBD |
| V483A | Up/Down conformations |
| V6151/F | In SARS-CoV ADE epitope |
| A831V | Potential fusion peptide in S2 |
| D839Y/N/E | S2 subunit |
| S943P | Fusion core of HR1 |
| P1263L | Cytoplasmic Tail |

Further SARS-COV-2 S glycoprotein mutations include: L18F, HV 69-70 deletion, Y144 deletion, E154Q, Q218E, A222V, S447N, F490S, S494P, N501Y, A570D, E583D, T618E, P681H, A701V, T716I, T723I, 1843V, S982A and D1118H. In late 2020, new SARS-COV-2 variants emerged in the UK, South Africa, Brazil and California that contained multiple mutations. The mutations present in the SARS-COV-2 S glycoprotein in the UK variant (named lineage B.1.1.7) include a H69 deletion (ΔH69), V70 deletion (ΔV70), a Y144 deletion (ΔY144), N501Y, A570D, P681H, T716I, S982A and D1118H (Rambaut et al. 2020 https://virological.org/t/preliminary-genomic-characterisation-of-an-emergent-sars-cov-2-lineage-in-the-uk-defined-by-a-novel-set-of-spike-mutations/563). In October 2020, the South African variant (named lineage B.1.351) includes six mutations in the SARS-COV-2 S glycoprotein protein-D80A, K417N, E484K, N501Y, D614G and A701V. By the end of November, three further SARS-COV-2 S glycoprotein mutations had emerged (L18F, R246I and K417N) and the deletion of three amino acids at L242 (ΔL242), A243 (ΔA243) and L244 (ΔL244) (Tegally et al. (2020) https://doi.org/10.1101/2020.12.21.20248640). The mutations present in the SARS-COV-2 S glycoprotein in the Brazilian variant (named linage P.1) include L18F, T20N, P26S, D138Y, R190S, K417T, E484K, N501Y, H655Y, T1027I and V1176F. The mutations present in the SARS-COV-2 S glycoprotein in the Californian variant (known as CAL.20C) include S13I, W152C and L452R (Zhang et al. (2021) https://doi.org/10.1101/2021.01.18.21249786).

In some embodiments, the amino acid sequence of the full-length SARS-COV-2 S glycoprotein can have multiple mutations. For example, two or more, three or more, four or more, five or more, six or more, seven or more, eight or more, nine or more, ten or more of mutations relative to the amino acid sequence of SEQ ID NO: 1. The mutations in the SARS-COV-2 S glycoprotein can be amino acid deletions or amino acid substitutions. Possible combinations of mutations include: (a) L18F, A222V, D614G; (b) A222V, D614G; (c) A222V, E583D, D614G; (d) S447N, D614G; (e) E154Q, F490S, D614G, 1834V; (f) D614G, A701V; (g) Q218E, D614G; (h) D614G, T618R; (i) ΔL242, ΔA243, ΔL244; (j) A222V, E583D, A701V; (k) ΔH69, ΔV70, ΔY144, N501Y, A570D, D614G, P681H, T716I, S982A, D1118H (UK variant+D614G); (1) D80A, K417N, E484K, N501Y, D614G and A701V (South African fixed mutations+D614G); (m) D80A, K417N, E484K, N501Y and A701V (South African fixed mutations; (n) D80A, D215G, ΔL242, ΔA243, ΔL244, A417V, E484K, N501Y, D614G, A701V (South African variant 1+D614G); (o) L18F, D80A, D215G, ΔL242, ΔA243, ΔL244, K417N, E484K, N501Y, D614G and A701V (South African variant 2+D614G); (p) L18F, T20N, P26S, D138Y, R190S, K417T, E484K, N501Y, D614G, H655Y, T1027I and V1176F (the Brazilian variant+D614G) and (q) S13I, W152C, L452R and D614G (Californian variant+D614G).

In some embodiments, the amino acid sequence of the full-length SARS-COV-2 S glycoprotein can have one or more of mutations relative to the amino acid sequence of SEQ ID NO: 1. This may include one or more of the following mutations: D614G mutation, L5F mutation, L8V/W mutation, H49Y mutation, Y145H/del mutation, Q239K mutation, V367F mutation, G476S mutation, V483A mutation, V6151/F mutation, A831V mutation, D839Y/N/E mutation, S943P mutation, P1263L mutation. Accordingly, in particular embodiments, any of the S proteins or antigenic fragments thereof described herein comprises a D614G mutation. For example, in particular embodiments, the SARS-COV-2 spike protein, the ectodomain thereof or the antigenic fragment thereof comprises a D614G mutation.

In some embodiments, any of the S proteins or antigenic fragments thereof described herein comprises the L5F mutation. In some embodiments, any of the S proteins or antigenic fragments thereof described herein comprises the L8V/W mutation. In some embodiments, any of the S proteins or antigenic fragments thereof described herein comprises the H49Y mutation. In some embodiments, any of the S proteins or antigenic fragments thereof described herein comprises the Y145H/del mutation. In some embodiments, any of the S proteins or antigenic fragments thereof described herein comprises the Q239K mutation. In some embodiments, any of the S proteins or antigenic fragments thereof described herein comprises the V367F mutation. In some embodiments, any of the S proteins or antigenic fragments thereof described herein comprises the G476S mutation. In some embodiments, any of the S proteins or antigenic fragments thereof described herein comprises the V483A mutation. In some embodiments, any of the S proteins or antigenic fragments thereof described herein comprises the V6151/F mutation. In some embodiments, any of the S proteins or antigenic fragments thereof described herein comprises the A831V mutation. In some embodiments, any of the S proteins or antigenic fragments thereof described herein comprises the D839Y/N/E mutation. In some embodiments, any of the S proteins or antigenic fragments thereof described herein comprises the S943P mutation. In some embodiments, any of the S proteins or antigenic fragments thereof described herein comprises the P1263L mutation.

An optimized nucleotide sequence according to the present invention may encode the SARS-COV-2 S protein or an antigenic fragment thereof. In particular embodiments, the optimized nucleotide sequence encodes a full-length SARS-COV-2 S protein. The full-length SARS-COV-2 S protein can have the amino acid sequence comprising SEQ ID NO: 1 or an amino acid sequence at least 95%, 96%, 97%, 98%, or 99% identical to SEQ ID NO: 1. In some embodiment, the optimized nucleotide sequence encoding the full-length SARS-COV-2 S protein has the sequence of SEQ ID NO: 29. In other embodiments, the optimized nucleotide sequence is at least 60%, 65%, 70%, 75%, 80%, 85%, 90%, 95%, 96%, 97%, 98%, or 99% identical to SEQ ID NO:29 and encodes the amino acid sequence of SEQ ID NO:1.

In some embodiments, the optimized nucleotide sequence encodes a SARS-COV-2 S protein comprising one or more mutations relative to the amino acid sequence of SEQ ID NO: 1. For example, in some embodiments, the optimized nucleotide sequence encodes a SARS-COV2 S protein comprising one or more of the following mutations: D614G mutation, L5F mutation, L8V/W mutation, H49Y mutation, Y145H/del mutation, Q239K mutation, V367F mutation, G476S mutation, V483A mutation, V6151/F mutation, A831V mutation, D839Y/N/E mutation, S943P mutation, P1263L mutation. Accordingly, in particular embodiments, the optimized nucleotide sequence encodes a SARS-COV2 S protein comprising the D614G mutation. For example, in particular embodiments the optimized nucleotide sequence encodes a SARS-COV-2 spike protein, an ectodomain thereof or an antigenic fragment thereof which comprises the D614G mutation.

In some embodiments, the optimized nucleotide sequence encodes a SARS-COV2 S protein comprising the L5F mutation. In some embodiments, the optimized nucleotide sequence encodes a SARS-COV2 S protein comprising the L8V/W mutation. In some embodiments, the optimized nucleotide sequence encodes a SARS-COV2 S protein comprising the H49Y mutation. In some embodiments, the optimized nucleotide sequence encodes a SARS-COV2 S protein comprising the Y145H/del mutation. In some embodiments, the optimized nucleotide sequence encodes a SARS-COV2 S protein comprising the Q239K mutation. In some embodiments, the optimized nucleotide sequence encodes a SARS-COV2 S protein comprising the V367F mutation. In some embodiments, the optimized nucleotide sequence encodes a SARS-COV2 S protein comprising the G467S mutation. In some embodiments, the optimized nucleotide sequence encodes a SARS-COV2 S protein comprising the V483A mutation. In some embodiments, the optimized nucleotide sequence encodes a SARS-COV2 S protein comprising the V6151/F mutation. In some embodiments, the optimized nucleotide sequence encodes a SARS-COV2 S protein comprising the A831V mutation. In some embodiments, the optimized nucleotide sequence encodes a SARS-COV2 S protein comprising the D839Y/N/E mutation. In some embodiments, the optimized nucleotide sequence encodes a SARS-COV2 S protein comprising the S943P mutation. In some embodiments, the optimized nucleotide sequence encodes a SARS-CoV2 S protein comprising the P1263L mutation.

Alternatively, an optimized nucleotide sequence according to the present invention may encode an antigenic fragment of the SARS-COV-2 S protein. In certain embodiments, the optimized nucleotide sequence may encode the ectodomain of the SARS-COV-2 S protein, which can have the amino acid sequence of SEQ ID NO:2 or an amino acid sequence at least 95%, 96%, 97%, 98%, or 99% identical to SEQ ID NO: 2. The ectodomain does not contain residues 1209-1273 of the full length SARS-COV-2 S protein, which includes the transmembrane domain and the cytoplasmic tail. In some embodiments, the optimized nucleotide sequence encoding the ectodomain of the SARS-COV-2 S protein has the sequence of SEQ ID NO: 30. In other embodiments, the optimized nucleotide sequence is at least 60%, 65%, 70%, 75%, 80%, 85%, 90%, 95%, 96%, 97%, 98%, or 99% identical to SEQ ID NO: 30 and encodes the amino acid sequence of SEQ ID NO: 2.

In other embodiments, an antigenic fragment of the SARS-COV-2 S protein may comprise one or more of the S1 subunit, the S2 subunit and/or the S2′ subunit of the SARS-COV-2 S protein. For example, the optimized nucleotide sequence may encode the S1 subunit, which has the amino acid sequence of SEQ ID NO: 3. Accordingly, in one embodiment, the optimized nucleotide sequence may encode an amino acid sequence comprising SEQ ID NO:3. In one embodiment, an optimized nucleotide sequence encoding the S1 subunit of the SARS-COV-2 S protein has the sequence of SEQ ID NO: 31. In other embodiments, the optimized nucleotide sequence is at least 60%, 65%, 70%, 75%, 80%, 85%, 90%, 95%, 96%, 97%, 98%, or 99% identical to SEQ ID NO: 31 and encodes the amino acid sequence of SEQ ID NO: 3. In an alternative embodiment, the optimized nucleotide sequence may encode the S2 subunit, which has the amino acid sequence of SEQ ID NO: 4. Accordingly, in one embodiment, the optimized nucleotide sequence may encode an amino acid sequence comprising SEQ ID NO: 4. In one embodiment, an optimized nucleotide sequence encoding the S2 subunit of the SARS-COV-2 S protein has the sequence of SEQ ID NO: 32. In other embodiments, the optimized nucleotide sequence is at least 60%, 65%, 70%, 75%, 80%, 85%, 90%, 95%, 96%, 97%, 98%, or 99% identical to SEQ ID NO: 32 and encodes the amino acid sequence of SEQ ID NO: 4. In an alternative embodiment, the optimized nucleotide sequence may encode the S2′ subunit, which has the amino acid sequence of SEQ ID NO: 5. Accordingly, in one embodiment, the optimized nucleotide sequence may encode an amino acid sequence comprising SEQ ID NO: 5. In one embodiment, an optimized nucleotide sequence encoding the S2′ subunit of the SARS-COV-2 S protein has the sequence of SEQ ID NO: 33. In other embodiments, the optimized nucleotide sequence is at least 60%, 65%, 70%, 75%, 80%, 85%, 90%, 95%, 96%, 97%, 98%, or 99% identical to SEQ ID NO: 33 and encodes the amino acid sequence of SEQ ID NO: 5.

In some embodiments, an antigenic fragment of the SARS-COV-2 S protein may comprise the full length S2 subunit or S2′ subunit of the SARS-COV-2 S protein. The full length S2 subunit or S2′ subunit comprises the transmembrane domain and the cytoplasmic tail. The full length S2 subunit encompasses residues 686 to 1273 of the SARS-COV-2 S protein and the S2′ subunit encompasses residues 816 to 1273 of the SARS-COV-2 S protein. For example, the optimized nucleotide sequence may encode the full length S2 subunit, which has the amino acid sequence of SEQ ID NO:72. Accordingly, in one embodiment, the optimized nucleotide sequence may encode an amino acid sequence comprising SEQ ID NO:72. In one embodiment, an optimized nucleotide sequence encoding the full length S2 subunit of the SARS-COV-2 S protein has the sequence of SEQ ID NO: 71. In other embodiments, the optimized nucleotide sequence is at least 60%, 65%, 70%, 75%, 80%, 85%, 90%, 95%, 96%, 97%, 98%, or 99% identical to SEQ ID NO:71 and encodes the amino acid sequence of SEQ ID NO: 72. In an alternative embodiment, the optimized nucleotide sequence may encode the full length S2′ subunit, which has the amino acid sequence of SEQ ID NO: 98. Accordingly, in one embodiment, the optimized nucleotide sequence may encode an amino acid sequence comprising SEQ ID NO: 98. In one embodiment, an optimized nucleotide sequence encoding the full length S2′ subunit of the SARS-COV-2 S protein has the sequence of SEQ ID NO:97. In other embodiments, the optimized nucleotide sequence is at least 81 and encodes the amino acid sequence of SEQ ID NO:98.

The SARS-COV-2 S protein mediates viral entry into host cells by first binding to the angiotensin-converting enzyme 2 (ACE2) receptor through the receptor-binding domain (RBD), which is located in the S1 subunit, and then fusing the viral and host membranes through the S2 subunit (Tai et al. (2020) Cellular and Molecular immunology, doi.org/10.1038/s41423-020-0400-4). Tai et al. identified a region of the RBD of SARS-COV-2 at residues 331 to 524 of the S protein. A putative RBD from residues 331 to 521 of the SARS-COV-2 S protein is provided by SEQ ID NO: 6 in Table 2 below. A recombinant fusion protein containing 193-amino acid RBD (residues 318-510) of SARS-COV and a human IgG1 Fc fragment has been shown to induce highly potent antibody responses in rabbits immunized with it (He et al. (2004) Biochem Biophys Res Commun; 324 (2): 773-781.). Therefore, the RBD of SARS-COV-2 S protein may also be able to highly induce an antibody response. Both the RBD of SARS-COV and the RBD of SARS-COV-2 bind to ACE2. Therefore, it is contemplated that the antigenic fragment of the SARS-COV-2 S protein may encode the RBD. Accordingly, in particular embodiments, the optimized nucleotide sequence may encode an amino acid sequence comprising the RBD of the SARS-COV-2 S protein, which has the amino acid sequence of SEQ ID NO: 6. In one embodiment, an optimized nucleotide sequence encoding the RBD of the SARS-COV-2 S protein has the sequence of SEQ ID NO: 34. In other embodiments, the optimized nucleotide sequence is at least 60%, 65%, 70%, 75%, 80%, 85%, 90%, 95%, 96%, 97%, 98%, or 99% identical to SEQ ID NO: 34 and encodes the amino acid sequence of SEQ ID NO: 6.

In certain embodiments, the antigenic fragment of the SARS-COV-2 S protein is fused with an exogenous N-terminal signal peptide. The signal peptide targets the protein to the ER and the secretory pathway, so that the protein enters the secretory pathway in the host cell in which it is expressed. In particular embodiments, the invention provides an antigenic fragment of the SARS-COV-2 S protein operably linked with an N-terminal signal peptide. For example, the RBD of the SARS-COV-2 S protein may be operably linked to the N-terminal signal peptide, which enables the resulting protein to be secreted from the host cell expressing it.

In specific embodiments, the N-terminal signal peptide can have the sequence MFVFLVLLPLVSSQC (SEQ ID NO: 7), which is the native signal peptide of the naturally occurring SARS-COV-2 S protein. In some embodiments, the signal peptide is encoded by the nucleotide sequence ATGTTCGTCTTCCTCGTGCTGCTCCCACTCGTTTCTTCCCAGTGT (SEQ ID NO: 37). Numerous other signal peptides are known in the art, which can be used to secrete a protein from a host cell, for example those mentioned in the review by Freudl (2018) Microbial Cell Factories 17:52. An alternative signal peptide that can be used as part of the invention is MATGSRTSLLLAFGLLCLPWLQEGSAFPTIPLS (SEQ ID NO:38). In some embodiments, the signal peptide is encoded by the nucleotide sequence AUGGCCACUGGAUCAAGAACCUCACUGCUGCUCGCUUUUGGACUGCUUUGCCUGC CCUGGUUGCAAGAAGGAUCGGCUUUCCCGACCAUCCCACUCUCC (SEQ ID NO: 39). Another signal peptide that can be used as part of the invention is MATGSRTSLLLAFGLLCLPWLQEGSAFPTIPLS (SEQ ID NO:40). In some embodiments, the signal peptide is encoded by the nucleotide sequence AUGGCAACUGGAUCAAGAACCUCCCUCCUGCUCGCAUUCGGCCUGCUCUGUCUCC CAUGGCUCCAAGAAGGAAGCGCGUUCCCCACUAUCCCCCUCUCG (SEQ ID NO:41).

The original annotation of the SARS-COV-2 genome identified the signal peptide sequence of the SARS-COV-2 S protein as being SEQ ID NO: 7. An alternative annotation of the SARS-COV-2 genome identified a longer native N-terminal signal peptide sequence, MFLLTTKRTMFVFLVLLPLVSSQC (SEQ ID NO: 142), which is nine amino acids longer. In specific embodiments, the N-terminal signal peptide can be has the sequence of SEQ ID NO: 142.

In some embodiments, the signal peptide is encoded by the nucleotide sequence ATGTTCCTGCTGACAACAAAAAGAACCATGTTTGTGTTCCTGGTGCTGCTGCCTCTG GTGTCCTCACAGTGT (SEQ ID NO: 143).

In particular embodiments, the optimized nucleotide sequence of the invention can encode an amino acid sequence comprising the RBD of the SARS-COV-2 S protein operably linked with an N-terminal signal peptide, which has the amino acid sequence comprising SEQ ID NO: 8. In one embodiment, an optimized nucleotide sequence encoding the RBD of the SARS-COV-2 S protein operably linked with an N-terminal signal peptide has the sequence of SEQ ID NO: 35. In other embodiments, the optimized nucleotide sequence is at least 60%, 65%, 70%, 75%, 80%, 85%, 90%, 95%, 96%, 97%, 98%, or 99% identical to SEQ ID NO: 35 and encodes the amino acid sequence of SEQ ID NO: 8.

In particular embodiments, the optimized nucleotide sequence of the invention can encode the S2 subunit of the SARS-COV-2 S protein operably linked with an N-terminal signal peptide, which has the amino acid sequence comprising SEQ ID NO:74. In one embodiment, an optimized nucleotide sequence encoding the S2 subunit of the SARS-COV-2 S protein operably linked with an N-terminal signal peptide has the sequence of SEQ ID NO: 73. In other embodiments, the optimized nucleotide sequence is at least 60%, 65%, 70%, 75%, 80%, 85%, 90%, 95%, 96%, 97%, 98%, or 99% identical to SEQ ID NO: 73 and encodes the amino acid sequence of SEQ ID NO: 74.

In particular embodiments, the optimized nucleotide sequence of the invention can encode the S2′ subunit of the SARS-COV-2 S protein operably linked with an N-terminal signal peptide, which has the amino acid sequence comprising SEQ ID NO:66. In one embodiment, an optimized nucleotide sequence encoding the S2 subunit of the SARS-COV-2 S protein operably linked with an N-terminal signal peptide has the sequence of SEQ ID NO: 65. In other embodiments, the optimized nucleotide sequence is at least 60%, 65%, 70%, 75%, 80%, 85%, 90%, 95%, 96%, 97%, 98%, or 99% identical to SEQ ID NO: 65 and encodes the amino acid sequence of SEQ ID NO:66.

In particular embodiments, the optimized nucleotide sequence of the invention can encode the full length S2 subunit of the SARS-COV-2 S protein operably linked with an N-terminal signal peptide, which has the amino acid sequence comprising SEQ ID NO:68. In one embodiment, an optimized nucleotide sequence encoding the full length S2 subunit of the SARS-COV-2 S protein operably linked with an N-terminal signal peptide has the sequence of SEQ ID NO: 67. In other embodiments, the optimized nucleotide sequence is at least 60%, 65%, 70%, 75%, 80%, 85%, 90%, 95%, 96%, 97%, 98%, or 99% identical to SEQ ID NO: 67 and encodes the amino acid sequence of SEQ ID NO:68. In particular embodiments, the optimized nucleotide sequence of the invention can encode the full length S2′ subunit of the SARS-COV-2 S protein operably linked with an N-terminal signal peptide, which has the amino acid sequence comprising SEQ ID NO:96. In one embodiment, an optimized nucleotide sequence encoding the full length S2′ subunit of the SARS-COV-2 S protein operably linked with an N-terminal signal peptide has the sequence of SEQ ID NO: 95. In other embodiments, the optimized nucleotide sequence is at least 60%, 65%, 70%, 75%, 80%, 85%, 90%, 95%, 96%, 97%, 98%, or 99% identical to SEQ ID NO: 95 and encodes the amino acid sequence of SEQ ID NO:96.

CoV S proteins are typical class I viral fusion proteins, which require protease cleavage in order for the fusion potential of S protein to be activated. A two-step sequential protease cleavage model has been proposed for activation of S proteins of SARS-COV-2 S protein, (1) priming cleavage between the S1 and S2 subunits and (2) activating cleavage on the S2′ site (Ou et al. (2020) Nature communications, 11, 1620). The SARS-COV-2 S protein harbors a furin cleavage site at the boundary between the S1/S2 subunits, which is processed during biogenesis, which sets this virus apart from SARS-COV and SARS-related CoVs (Walls et al. (2020) Cell doi.org/10.1016/j.cell.2020.02.058).

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation, by mutating residues 986 and 987 to proline and which contains an extended N-terminal signal peptide. For example, an optimized nucleotide sequence may encode a prefusion stabilized SARS-COV-2 S protein, which has an amino acid sequence comprising SEQ ID NO: 123. In one embodiment, the optimized nucleotide sequence has the sequence of SEQ ID NO: 122. In other embodiments, the optimized nucleotide sequence is at least 60%, 65%, 70%, 75%, 80%, 85%, 90%, 95%, 96%, 97%, 98%, or 99% identical to SEQ ID NO: 122 and encodes the amino acid sequence of SEQ ID NO: 123.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation, by mutating residues 817, 892, 899, 942, 986 and 987 to proline and which contains an extended N-terminal signal peptide. For example, an optimized nucleotide sequence may encode a prefusion stabilized SARS-COV-2 S protein, which has an amino acid sequence comprising SEQ ID NO: 137. In one embodiment, the optimized nucleotide sequence has the sequence of SEQ ID NO: 136. In other embodiments, the optimized nucleotide sequence is at least 60%, 65%, 70%, 75%, 80%, 85%, 90%, 95%, 96%, 97%, 98%, or 99% identical to SEQ ID NO: 136 and encodes the amino acid sequence of SEQ ID NO: 137.

Prefusion stabilization tends to increase the recombinant expression of viral fusion glycoproteins, possibly by preventing misfolding that results from a tendency of such proteins to adopt the more stable postfusion structure. Prefusion-stabilized viral glycoproteins are considered superior immunogens to their wild-type counterparts.

A prefusion stabilized conformation of the SARS-COV-2 S protein can be created by mutating the furin cleavage site in order to prevent the cleavage of the S1 and S2 subunits. For example, the RRAR residues in the furin cleavage site (positions 682-685) can be mutated to GSAS residues (i.e. R682G R683S A684A R685S). Accordingly, in some embodiments, an optimized nucleotide sequence in accordance with the invention may encode a prefusion stabilized SARS-CoV-2 S protein, a prefusion stabilized ectodomain of the SARS-COV-2 S protein, or an antigenic fragment of either, in which has been modified relative to naturally occurring SARS-COV-2 S protein by removing the furin cleavage site required for activation, e.g., by replacing the amino acid residues recognized by furin with alternative amino acids that do not form a furin cleavage site but maintain the structure of the S protein. In a specific embodiment, the RRAR furin cleavage site residues 682-685 can be mutated to the residues GSAS to remove the furin cleavage site. In particular embodiments, an optimized nucleotide sequence may encode a prefusion stabilized SARS-COV-2 S protein, which has an amino acid sequence comprising SEQ ID NO: 9. In one embodiment, an optimized nucleotide sequence encoding a prefusion stabilized SARS-COV-2 S protein has the sequence of SEQ ID NO: 42. In other embodiments, the optimized nucleotide sequence is at least 60%, 65%, 70%, 75%, 80%, 85%, 90%, 95%, 96%, 97%, 98%, or 99% identical to SEQ ID NO: 42 and encodes the amino acid sequence of SEQ ID NO: 9.

The SARS-COV-2 S protein can be stabilized in its prefusion conformation by substituting one or more of residues 985, 986 and 987 (i.e., D985P) with proline. For example, a prefusion stabilized conformation of the SARS-COV-2 S protein can be created by making one stabilizing proline mutation at residue 985 (i.e., D985P); two stabilizing proline mutations at residues 986 and 987 (i.e., K986P, V987P); or three stabilizing proline mutations at residues 985, 986 and 987 (i.e., D985P, K986P, V987P).

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by mutating residues 986 and 987 to proline. For example, an optimized nucleotide sequence may encode a prefusion stabilized SARS-COV-2 S protein, which has an amino acid sequence comprising SEQ ID NO:10. In one embodiment, the optimized nucleotide sequence has the sequence of SEQ ID NO: 43. In other embodiments, the optimized nucleotide sequence is at least 60%, 65%, 70%, 75%, 80%, 85%, 90%, 95%, 96%, 97%, 98%, or 99% identical to SEQ ID NO: 43 and encodes the amino acid sequence of SEQ ID NO: 10. In further embodiments, an optimized nucleotide sequence may encode a prefusion stabilized SARS-COV-2 S protein, which has an amino acid sequence comprising SEQ ID NO: 118. This amino acid sequence comprises the D614G mutation. In one embodiment, the optimized nucleotide sequence has the sequence of SEQ ID NO: 119. In specific embodiments, the optimized nucleotide sequence is at least 60%, 65%, 70%, 75%, 80%, 85%, 90%, 95%, 96%, 97%, 98%, or 99% identical to SEQ ID NO: 119 and encodes the amino acid sequence of SEQ ID NO: 118.

In certain embodiments, an optimized nucleotide sequence may encode a prefusion stabilized variant of the S2 subunit of the SARS-COV-2 S protein, which has an amino acid sequence comprising SEQ ID NO:78. In one embodiment, the optimized nucleotide sequence has the sequence of SEQ ID NO: 77. In other embodiments, the optimized nucleotide sequence is at least 60%, 65%, 70%, 75%, 80%, 85%, 90%, 95%, 96%, 97%, 98%, or 99% identical to SEQ ID NO: 77 and encodes the amino acid sequence of SEQ ID NO:78. In certain embodiments, an optimized nucleotide sequence may encode a prefusion stabilized variant of the full length S2 subunit of the SARS-COV-2 S protein, which has an amino acid sequence comprising SEQ ID NO: 70. In one embodiment, the optimized nucleotide sequence has the sequence of SEQ ID NO: 69. In other embodiments, the optimized nucleotide sequence is at least 60%, 65%, 70%, 75%, 80%, 85%, 90%, 95%, 96%, 97%, 98%, or 99% identical to SEQ ID NO:69 and encodes the amino acid sequence of SEQ ID NO: 70.

In certain embodiments, an optimized nucleotide sequence may encode a prefusion stabilized variant of the S2′ subunit of the SARS-COV-2 S protein, which has an amino acid sequence comprising SEQ ID NO:82. In one embodiment, the optimized nucleotide sequence has the sequence of SEQ ID NO:81. In other embodiments, the optimized nucleotide sequence is at least 60%, 65%, 70%, 75%, 80%, 85%, 90%, 95%, 96%, 97%, 98%, or 99% identical to SEQ ID NO: 81 and encodes the amino acid sequence of SEQ ID NO:82. In certain embodiments, an optimized nucleotide sequence may encode a prefusion stabilized variant of the full length S2′ subunit of the SARS-COV-2 S protein, which has an amino acid sequence comprising SEQ ID NO: 86. In one embodiment, the optimized nucleotide sequence has the sequence of SEQ ID NO: 85. In other embodiments, the optimized nucleotide sequence is at least 60%, 65%, 70%, 75%, 80%, 85%, 90%, 95%, 96%, 97%, 98%, or 99% identical to SEQ ID NO:85 and encodes the amino acid sequence of SEQ ID NO:86.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by mutating residue 985 to proline. For example, an optimized nucleotide sequence may encode a prefusion stabilized SARS-COV-2 S protein, which has an amino acid sequence comprising SEQ ID NO:88. In one embodiment, the optimized nucleotide sequence has the sequence of SEQ ID NO: 87. In other embodiments, the optimized nucleotide sequence is at least 60%, 65%, 70%, 75%, 80%, 85%, 90%, 95%, 96%, 97%, 98%, or 99% identical to SEQ ID NO: 87 and encodes the amino acid sequence of SEQ ID NO: 88.

In some embodiments, a prefusion stabilized conformation of the SARS-COV-2 S protein can be created by making three stabilizing proline mutations in the C-terminal of the S2 subunit at residues 985, 986 and 987 (i.e., D985P, K986P, V987P). In some embodiments, an optimized nucleotide sequence according to the present invention may encode a prefusion stabilized SARS-CoV-2 S protein, a prefusion stabilized ectodomain of the SARS-COV-2 S protein, or an antigenic fragment of either, which has been modified relative to naturally occurring SARS-COV-2 S protein by mutating residues 985, 986 and 987 to proline. For example, an optimized nucleotide sequence may encode a prefusion stabilized SARS-COV-2 S protein, which has an amino acid sequence comprising SEQ ID NO:92. In one embodiment, the optimized nucleotide sequence has the sequence of SEQ ID NO: 91. In other embodiments, the optimized nucleotide sequence is at least 60%, 65%, 70%, 75%, 80%, 85%, 90%, 95%, 96%, 97%, 98%, or 99% identical to SEQ ID NO: 91 and encodes the amino acid sequence of SEQ ID NO: 92.

In some embodiments, a prefusion stabilized conformation of the SARS-COV-2 S protein can be created by mutating the furin cleavage site in order to prevent the cleavage of the SI and S2 subunits and (a) by making two stabilizing proline mutations at residues 986 and 987 (i.e., K986P, V987P) and/or (b) by making a stabilizing proline mutation at residue 985. For example, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation and by mutating residues 986 and 987 to proline. In some embodiments, the residues forming the furin cleavage site at residues 682-685 are mutated to the residues GSAS. For example, an optimized nucleotide sequence may encode a prefusion stabilized SARS-COV-2 S protein, which has an amino acid sequence comprising SEQ ID NO:11. In one embodiment, the optimized nucleotide sequence has the sequence of SEQ ID NO: 44. In other embodiments, the optimized nucleotide sequence is at least 60%, 65%, 70%, 75%, 80%, 85%, 90%, 95%, 96%, 97%, 98%, or 99% identical to SEQ ID NO: 44 and encodes the amino acid sequence of SEQ ID NO: 11. In further embodiments, an optimized nucleotide sequence may encode a prefusion stabilized SARS-COV-2 S protein, which has an amino acid sequence comprising SEQ ID NO: 120. This amino acid sequence comprises the D614G mutation. In one embodiment, the optimized nucleotide sequence has the sequence of SEQ ID NO: 121. In some embodiments, the optimized nucleotide sequence is at least 60%, 65%, 70%, 75%, 80%, 85%, 90%, 95%, 96%, 97%, 98%, or 99% identical to SEQ ID NO: 121 and encodes the amino acid sequence of SEQ ID NO: 120. Alternatively, the optimized nucleotide sequence encodes a prefusion stabilized ectodomain of the SARS-COV-2 S protein which has been modified relative to naturally occurring SARS-COV-2 S protein, which has an amino acid sequence comprising SEQ ID NO:12. In one embodiment, the optimized nucleotide sequence has the sequence of SEQ ID NO: 45. In other embodiments, the optimized nucleotide sequence is at least 60%, 65%, 70%, 75%, 80%, 85%, 90%, 95%, 96%, 97%, 98%, or 99% identical to SEQ ID NO: 45 and encodes the amino acid sequence of SEQ ID NO: 12.

In certain embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation and by mutating residue 985 to proline. In some embodiments, the residues forming the furin cleavage site at residues 682-685 are mutated to the residues GSAS. For example, an optimized nucleotide sequence may encode a prefusion stabilized SARS-COV-2 S protein, which has an amino acid sequence comprising SEQ ID NO:90. In one embodiment, the optimized nucleotide sequence has the sequence of SEQ ID NO: 89. In other embodiments, the optimized nucleotide sequence is at least 60%, 65%, 70%, 75%, 80%, 85%, 90%, 95%, 96%, 97%, 98%, or 99% identical to SEQ ID NO: 89 and encodes the amino acid sequence of SEQ ID NO: 90.

In certain embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation and by mutating residues 985, 986 and 987 to proline. In some embodiments, the residues forming the furin cleavage site at residues 682-685 are mutated to the residues GSAS. For example, an optimized nucleotide sequence may encode a prefusion stabilized SARS-COV-2 S protein, which has an amino acid sequence comprising SEQ ID NO:94. In one embodiment, the optimized nucleotide sequence has the sequence of SEQ ID NO: 93. In other embodiments, the optimized nucleotide sequence is at least 60%, 65%, 70%, 75%, 80%, 85%, 90%, 95%, 96%, 97%, 98%, or 99% identical to SEQ ID NO: 93 and encodes the amino acid sequence of SEQ ID NO: 94.

The SARS-COV-2 S protein can be further stabilized in its prefusion conformation by substituting one or more of residues 817, 892, 899 and 942 (i.e F817P, A892P, A899P and A942P) with proline. For example, a prefusion stabilized conformation of the SARS-COV-2 S protein can be created by making one stabilizing proline mutation at residue 817 (i.e., F817P); two stabilizing proline mutations at residues 817 and 892 (i.e., F817P, A892P,); or three stabilizing proline mutations at residues 817, 892, 899 (i.e., F817P, A892P, A899P,); or four stabilizing proline mutations at residues 817, 892, 899 and 942 (i.e. F817P, A892P, A899P, A942P). In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by mutating residues 817, 892, 899 and 942 to proline.

In preferred embodiments, a prefusion stabilized conformation of the SARS-COV-2 S protein can be created by making stabilizing proline mutations at residues 817, 892, 899, 942, 986. In some embodiments, the optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by mutating residues 817, 892, 899, 942, 986 and 987 to proline. For example, an optimized nucleotide sequence may encode a prefusion stabilized SARS-COV-2 S protein, which has an amino acid sequence comprising SEQ ID NO: 129. In one embodiment, the optimized nucleotide sequence has the sequence of SEQ ID NO: 128. In other embodiments, the optimized nucleotide sequence is at least 60%, 65%, 70%, 75%, 80%, 85%, 90%, 95%, 96%, 97%, 98%, or 99% identical to SEQ ID NO: 128 and encodes the amino acid sequence of SEQ ID NO: 129.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation and by mutating residues 817, 892, 899, 942, 986 and 987 to proline. For example, an optimized nucleotide sequence may encode a prefusion stabilized SARS-COV-2 S protein, which has an amino acid sequence comprising SEQ ID NO: 131. In one embodiment, the optimized nucleotide sequence has the sequence of SEQ ID NO: 130. In other embodiments, the optimized nucleotide sequence is at least 60%, 65%, 70%, 75%, 80%, 85%, 90%, 95%, 96%, 97%, 98%, or 99% identical to SEQ ID NO: 130 and encodes the amino acid sequence of SEQ ID NO: 131.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by mutating residues 817, 892, 899, 942, 986 and 987 to proline and which contains the D614G mutation. For example, an optimized nucleotide sequence may encode a prefusion stabilized SARS-COV-2 S protein, which has an amino acid sequence comprising SEQ ID NO: 133. In one embodiment, the optimized nucleotide sequence has the sequence of SEQ ID NO: 132. In other embodiments, the optimized nucleotide sequence is at least 60%, 65%, 70%, 75%, 80%, 85%, 90%, 95%, 96%, 97%, 98%, or 99% identical to SEQ ID NO: 132 and encodes the amino acid sequence of SEQ ID NO: 133.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation, by mutating residues 817, 892, 899, 942, 986 and 987 to proline and which contains the D614G mutation. For example, an optimized nucleotide sequence may encode a prefusion stabilized SARS-COV-2 S protein, which has an amino acid sequence comprising SEQ ID NO: 135. In one embodiment, the optimized nucleotide sequence has the sequence of SEQ ID NO: 134. In other embodiments, the optimized nucleotide sequence is at least 60%, 65%, 70%, 75%, 80%, 85%, 90%, 95%, 96%, 97%, 98%, or 99% identical to SEQ ID NO: 134 and encodes the amino acid sequence of SEQ ID NO: 135.

A T4 bacteriophage fibritin Foldon can be placed at the C terminus of an antigenic fragment the SARS-COV-2 S protein in order to help induce trimer formation. Foldons have been used to produce trimeric influenza hemagglutinin stem domains for use in influenza vaccines (Lu et al. (2014) PNAS, 111, 1, 124-130). The Foldon can have the amino acid sequence of GYIPEAPRDGQAYVRKDGEWVLLSTFL (SEQ ID NO: 13). Accordingly, optimized nucleotide sequences according to the present invention may encode an ectodomain of the SARS-CoV-2 S protein, or an antigenic fragment thereof, and a C terminal Foldon. In particular embodiments, the Foldon is placed at the C terminus of the ectodomain of the SARS-COV-2 S protein or the S2′ subunit of the SARS-COV-2 S protein. In one embodiment, the invention provides an optimized nucleotide sequence that encodes an amino acid sequence comprising the ectodomain of the SARS-COV-2 S protein with a C terminal Foldon, which has an amino acid sequence comprising SEQ ID NO:14. In one embodiment, the optimized nucleotide sequence has the sequence of SEQ ID NO: 46. In other embodiments, the optimized nucleotide sequence is at least 60%, 65%, 70%, 75%, 80%, 85%, 90%, 95%, 96%, 97%, 98%, or 99% identical to SEQ ID NO: 46 and encodes the amino acid sequence of SEQ ID NO: 14. The invention also provides an optimized nucleotide sequence that encodes an amino acid sequence comprising the S2 subunit of the SARS-COV-2 S protein with a C terminal Foldon, which has an amino acid sequence comprising SEQ ID NO: 76. In one embodiment, the optimized nucleotide sequence has the sequence of SEQ ID NO: 75. In other embodiments, the optimized nucleotide sequence is at least 60%, 65%, 70%, 75%, 80%, 85%, 90%, 95%, 96%, 97%, 98%, or 99% identical to SEQ ID NO: 75 and encodes the amino acid sequence of SEQ ID NO: 76. The invention also provides an optimized nucleotide sequence that encodes an amino acid sequence comprising the S2′ subunit of the SARS-COV-2 S protein with a C terminal Foldon, which has an amino acid sequence comprising SEQ ID NO: 15. In one embodiment, the optimized nucleotide sequence has the sequence of SEQ ID NO: 47. In other embodiments, the optimized nucleotide sequence is at least 60%, 65%, 70%, 75%, 80%, 85%, 90%, 95%, 96%, 97%, 98%, or 99% identical to SEQ ID NO: 47 and encodes the amino acid sequence of SEQ ID NO: 15.

In some embodiments, the optimized nucleotide sequence encodes a prefusion stabilized ectodomain of the SARS-COV-2 S protein with a C terminal Foldon, wherein the ectodomain has been modified relative to the ectodomain of the naturally occurring SARS-COV-2 S protein by removing the furin cleavage site required for activation and/or by mutating residues 986 and 987 to proline. In particular embodiments, an optimized nucleotide sequence encodes a prefusion stabilized ectodomain of the SARS-COV-2 S protein with a C terminal Foldon, which has an amino acid sequence comprising SEQ ID NO: 16. In one embodiment, the optimized nucleotide sequence has the sequence of SEQ ID NO: 48. In other embodiments, the optimized nucleotide sequence is at least 60%, 65%, 70%, 75%, 80%, 85%, 90%, 95%, 96%, 97%, 98%, or 99% identical to SEQ ID NO: 48 and encodes the amino acid sequence of SEQ ID NO: 16. In other particular embodiments, an optimized nucleotide sequence encodes a prefusion stabilized ectodomain of the SARS-COV-2 S protein with a C terminal Foldon, wherein the ectodomain been modified relative to naturally occurring SARS-COV-2 S protein by removing the furin cleavage site required for activation and by mutating residues 986 and 987 to proline. Accordingly, in a particular embodiment, an optimized nucleotide sequence encodes a prefusion stabilized ectodomain of the SARS-COV-2 S protein, which has an amino acid sequence comprising SEQ ID NO: 17. In one embodiment, the optimized nucleotide sequence has the sequence of SEQ ID NO: 49. In other embodiments, the optimized nucleotide sequence is at least 60%, 65%, 70%, 75%, 80%, 85%, 90%, 95%, 96%, 97%, 98%, or 99% identical to SEQ ID NO: 49 and encodes the amino acid sequence of SEQ ID NO: 17.

In some embodiments, the optimized nucleotide sequence encodes a prefusion stabilized ectodomain of the S2 or S2′ subunit of the SARS-COV-2 S protein with a C terminal Foldon, wherein compared to the naturally occurring SARS-COV-2 S protein residues 986 and 987 have been mutated to proline. Accordingly, in a particular embodiment, an optimized nucleotide sequence encodes a prefusion stabilized S2 subunit of the SARS-COV-2 S protein, which has the amino acid sequence comprising SEQ ID NO: 80. In one embodiment, the optimized nucleotide sequence has the sequence of SEQ ID NO: 79. In other embodiments, the optimized nucleotide sequence is at least 60%, 65%, 70%, 75%, 80%, 85%, 90%, 95%, 96%, 97%, 98%, or 99% identical to SEQ ID NO: 79 and encodes the amino acid sequence of SEQ ID NO: 80. Accordingly, in a particular embodiment, an optimized nucleotide sequence encodes a prefusion stabilized S2 subunit of the SARS-COV-2 S protein which has an amino acid sequence comprising SEQ ID NO: 84. In one embodiment, the optimized nucleotide sequence has the sequence of SEQ ID NO: 83. In other embodiments, the optimized nucleotide sequence is at least 60%, 65%, 70%, 75%, 80%, 85%, 90%, 95%, 96%, 97%, 98%, or 99% identical to SEQ ID NO: 83 and encodes the amino acid sequence of SEQ ID NO: 84.

The presence of the Fc domain in a protein markedly increases the plasma half-life of the protein and thereby prolongs the molecule's therapeutic activity. The Fc domain is also able to slow renal clearance of a protein from the blood stream and enables the protein to interact with Fc-receptors (FcRs) found on immune cells, a feature that may be advantageous for their use in vaccines. In addition, the Fc domain folds independently and can improve the solubility and stability of the partner molecule both in vitro and in vivo (Czajkowsky et al (2012) EMBO Mol Med. (10): 1015-1028). Accordingly, the invention also provides an optimized nucleotide sequence that encodes an amino acid sequence comprising the ectodomain of the SARS-COV-2 S protein or an antigenic fragment thereof with a C-terminal Fc domain. The Fc domain can comprise the following amino acid sequence: PKSCDKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALPAPIE KTISKAKGQPREPQVYTLPPSRDELTKNQVSLTCLVKGFYPSDIAVEWESNGQPENNYK TTPPVLDSDGSFFLYSKLTVDKSRWQQGNVFSCSVMHEALHNHYTQKSLSLSPGK (SEQ ID NO: 18). In particular embodiments, the antigenic fragment is the RBD of the SARS-COV-2 S protein. In some embodiments, an optimized nucleotide sequence encodes an amino acid sequence comprising the RBD of the SARS-COV-2 S protein and an Fc domain, which has an amino acid sequence comprising SEQ ID NO: 19. In one embodiment, the optimized nucleotide sequence has the sequence of SEQ ID NO: 50. In other embodiments, the optimized nucleotide sequence is at least 60%, 65%, 70%, 75%, 80%, 85%, 90%, 95%, 96%, 97%, 98%, or 99% identical to SEQ ID NO: 50 and encodes the amino acid sequence of SEQ ID NO: 19.

The invention also provides an optimized nucleotide sequence that encodes the ectodomain of the SARS-COV-2 S protein, or an antigenic fragment thereof, operably linked with an N-terminal signal peptide and a C-terminal Fc domain. The Fc can have the amino acid sequence of SEQ ID NO:18. The signal peptide can have the amino acid sequence of SEQ ID NO: 7. In particular embodiments, the antigenic fragment is the RBD of the SARS-COV-2 S protein. In some embodiments, the invention provides an optimized nucleotide sequence that encodes an amino acid sequence comprising the RBD of the SARS-COV-2 S protein operably linked with an N-terminal signal peptide and a C-terminal Fc domain, which has an amino acid sequence comprising SEQ ID NO: 20. In one embodiment, the optimized nucleotide sequence has the sequence of SEQ ID NO: 36. In other embodiments, the optimized nucleotide sequence is at least 60%, 65%, 70%, 75%, 80%, 85%, 90%, 95%, 96%, 97%, 98%, or 99% identical to SEQ ID NO: 36 and encodes the amino acid sequence of SEQ ID NO: 20.

The pharmacokinetic properties of antibodies are largely dictated by the pH-dependent binding of the Fc domain to the neonatal Fc receptor (FcRn). For example, Fc domains containing the amino acid substitutions M428L/N434S (LS mutant), M252Y/S254T/T256E (YTE mutant), or H433K/N434F (KF mutant) confer 10- to 12-fold higher affinity for FcRn at pH 5.8. This results in a large increase in antibody half-life (2- to 4-fold longer circulation times). Modifying the Fc region included in a fusion protein of the present invention can therefore extend its half-life in serum. An Fc variant containing L309D/Q311H/N434S (DHS) substitutions has been shown to further improve the pharmacokinetics of an antibody relative to both native IgG1 and the aforementioned variants (Lee et al. (2019) Nature communications, 10, 5031). Accordingly, in certain embodiments, the Fc region has been mutated compared to wild-type, using the EU numbering system based on human IGHG. For example, the L residue at position 309, the Q residue at 311 and the N residues at 434 can be mutated to D, H and S respectively (i.e. L309D; Q311H and N434S). The mutated Fc domain can comprise the following sequence:

(SEQ ID NO: 100)
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVDHHDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHSHYTQKSLSLSPGK.

In other embodiments, the M residue at position 428 and the N residue at 434 can be mutated to L and S respectively (i.e. M428L and N434S). The mutated Fc domain can comprise the following sequence:

(SEQ ID NO: 101)
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVLHEALHSHYTQKSLSLSPGK.

In other embodiments, the M residue at position 252, the S residue at 254 and the T residue at 256 can be mutated to Y, T and E respectively (i.e. M252Y, S254T and T256E). The mutated Fc domain can comprise the following sequence:

(SEQ ID NO: 102)
PKSCDKTHTCPPCPAPELLGGPSVFLFPPKPKDTLYITREPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHNHYTQKSLSLSPGK.

In other embodiments, the H residue at position 433 and the N residue at 434 can be mutated to K and F respectively (i.e. H433K and N434F). The mutated Fc domain can comprise the following sequence:

(SEQ ID NO: 103)
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALKFHYTQKSLSLSPGK.

Accordingly, the invention also provides an optimized nucleotide sequence that encodes an antigenic fragment of the SARS-COV-2 S protein, or an antigenic fragment thereof, operably linked with an N-terminal signal peptide and a C-terminal Fc domain. The Fc can have the amino acid sequence of SEQ ID NO: 100, SEQ ID NO: 101, SEQ ID NO: 102 or SEQ ID NO: 103. The signal peptide can have the amino acid sequence of SEQ ID NO:7. In particular embodiments, the antigenic fragment is the RBD of the SARS-COV-2 S protein.

In some embodiments, the invention provides an optimized nucleotide sequence that encodes an amino acid sequence comprising the RBD of the SARS-COV-2 S protein operably linked with an N-terminal signal peptide and a C-terminal mutated Fc domain, which has an amino acid sequence comprising SEQ ID NO:104. In one embodiment, the optimized nucleotide sequence has the sequence of SEQ ID NO: 105. In other embodiments, the optimized nucleotide sequence is at least 60%, 65%, 70%, 75%, 80%, 85%, 90%, 95%, 96%, 97%, 98%, or 99% identical to SEQ ID NO: 105 and encodes the amino acid sequence of SEQ ID NO: 104.

In some embodiments, the invention provides an optimized nucleotide sequence that encodes an amino acid sequence comprising the RBD of the SARS-COV-2 S protein operably linked with an N-terminal signal peptide and a C-terminal mutated Fc domain, which has an amino acid sequence comprising SEQ ID NO: 106. In one embodiment, the optimized nucleotide sequence has the sequence of SEQ ID NO: 107. In other embodiments, the optimized nucleotide sequence is at least 60%, 65%, 70%, 75%, 80%, 85%, 90%, 95%, 96%, 97%, 98%, or 99% identical to SEQ ID NO: 107 and encodes the amino acid sequence of SEQ ID NO: 106.

In some embodiments, the invention provides an optimized nucleotide sequence that encodes an amino acid sequence comprising the RBD of the SARS-COV-2 S protein operably linked with an N-terminal signal peptide and a C-terminal mutated Fc domain, which has an amino acid sequence comprising SEQ ID NO: 108. In one embodiment, the optimized nucleotide sequence has the sequence of SEQ ID NO: 109. In other embodiments, the optimized nucleotide sequence is at least 60%, 65%, 70%, 75%, 80%, 85%, 90%, 95%, 96%, 97%, 98%, or 99% identical to SEQ ID NO: 109 and encodes the amino acid sequence of SEQ ID NO: 108.

In some embodiments, the invention provides an optimized nucleotide sequence that encodes an amino acid sequence comprising the RBD of the SARS-COV-2 S protein operably linked with an N-terminal signal peptide and a C-terminal mutated Fc domain, which has an amino acid sequence comprising SEQ ID NO: 110. In one embodiment, the optimized nucleotide sequence has the sequence of SEQ ID NO: 111. In other embodiments, the optimized nucleotide sequence is at least 60%, 65%, 70%, 75%, 80%, 85%, 90%, 95%, 96%, 97%, 98%, or 99% identical to SEQ ID NO: 111 and encodes the amino acid sequence of SEQ ID NO: 110.

Coronaviruses assemble at and bud into the lumen of the endoplasmic reticulum (ER)-Golgi intermediate compartment (ERGIC). The cytoplasmic tail of the SARS-COV-2 S protein contains an ER retrieval signal (ERRS) that can move the S protein from the Golgi to the ER. This process is thought to accumulate S proteins at the ERGIC, which facilitates S protein incorporation into viral particles. The ER retrieval signal in the SARS COV S protein is a dibasic motif (KxHxx) in the cytoplasmic tail, which is similar to a canonical dilysine ER retrieval signal (McBride et al (2007) Journal Of Virology, 81, 5, 2418-2428).

Mutating the ER retrieval signal may prevent the virus from forming viral particles. Without wishing to be bound by any particular theory, the inventors believe that it is advantageous to remove the ER retrieval signals from SARS-COV-2 S proteins that are intended for the inclusion in a vaccine. Therefore, in some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by mutating the ER retrieval signal. For example, the KLHYT ER retrieval signal of the SARS-COV-2 S protein can be removed by mutating resides 1268 and 1270 to alanine (i.e., ALAYT).

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation, by mutating residues 986 and 987 to proline and by removing the ER retrieval signal. For example, an optimized nucleotide sequence may encode a prefusion stabilized SARS-COV-2 S protein, which has an amino acid sequence comprising SEQ ID NO: 125. In one embodiment, the optimized nucleotide sequence has the sequence of SEQ ID NO: 124. In other embodiments, the optimized nucleotide sequence is at least 60%, 65%, 70%, 75%, 80%, 85%, 90%, 95%, 96%, 97%, 98%, or 99% identical to SEQ ID NO: 124 and encodes the amino acid sequence of SEQ ID NO: 125.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation, by mutating residues 986 and 987 to proline, by removing the ER retrieval signal and which contains an extended N-terminal signal peptide. For example, an optimized nucleotide sequence may encode a prefusion stabilized SARS-COV-2 S protein, which has an amino acid sequence comprising SEQ ID NO: 127. In one embodiment, the optimized nucleotide sequence has the sequence of SEQ ID NO: 126. In other embodiments, the optimized nucleotide sequence is at least 60%, 65%, 70%, 75%, 80%, 85%, 90%, 95%, 96%, 97%, 98%, or 99% identical to SEQ ID NO: 126 and encodes the amino acid sequence of SEQ ID NO: 127.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation, by mutating residues 817, 892, 899, 942, 986 and 987 to proline and by removing the ER retrieval signal. For example, an optimized nucleotide sequence may encode a prefusion stabilized SARS-COV-2 S protein, which has an amino acid sequence comprising SEQ ID NO: 139. In one embodiment, the optimized nucleotide sequence has the sequence of SEQ ID NO: 138. In other embodiments, the optimized nucleotide sequence is at least 60%, 65%, 70%, 75%, 80%, 85%, 90%, 95%, 96%, 97%, 98%, or 99% identical to SEQ ID NO: 138 and encodes the amino acid sequence of SEQ ID NO: 139.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to naturally occurring SARS-COV-2 S protein by removing the furin cleavage site required for activation, by mutating residues 817, 892, 899, 942, 986 and 987 to proline, by removing the ER retrieval signal and which contains an extended N-terminal signal peptide. For example, an optimized nucleotide sequence may encode a prefusion stabilized SARS-COV-2 S protein, which has an amino acid sequence comprising SEQ ID NO: 141. In one embodiment, the optimized nucleotide sequence has the sequence of SEQ ID NO: 140. In other embodiments, the optimized nucleotide sequence is at least 60%, 65%, 70%, 75%, 80%, 85%, 90%, 95%, 96%, 97%, 98%, or 99% identical to SEQ ID NO: 140 and encodes the amino acid sequence of SEQ ID NO: 141.

A specific combination of mutations listed in paragraphs 0 and may be introduced in any of the SARS-COV-2 S proteins disclosed herein. For example, in specific embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) to contain the ΔH69, ΔV70, ΔY144, N501Y, A570D, D614G, P681H, T716I, S982A and D1118H mutations. Accordingly, in certain embodiments, an optimized nucleotide sequence of the invention may encode a prefusion stabilized SARS-COV-2 S protein, which has an amino acid sequence comprising SEQ ID NO: 151. In one embodiment, the optimized nucleotide sequence has the sequence of SEQ ID NO: 150. In other embodiments, the optimized nucleotide sequence is at least 60%, 65%, 70%, 75%, 80%, 85%, 90%, 95%, 96%, 97%, 98%, or 99% identical to SEQ ID NO: 150 and encodes the amino acid sequence of SEQ ID NO: 151.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) by mutating residues 986 and 987 to proline and which contains the ΔH69, ΔV70, ΔY144, N501Y, A570D, D614G, P681H, T716I, S982A and D1118H mutations (UK variant+D614G). Accordingly, in certain embodiments, an optimized nucleotide sequence of the invention may encode a prefusion stabilized SARS-COV-2 S protein, which has an amino acid sequence comprising SEQ ID NO: 153. In one embodiment, the optimized nucleotide sequence has the sequence of SEQ ID NO: 152. In other embodiments, the optimized nucleotide sequence is at least 60%, 65%, 70%, 75%, 80%, 85%, 90%, 95%, 96%, 97%, 98%, or 99% identical to SEQ ID NO: 152 and encodes the amino acid sequence of SEQ ID NO: 153.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) by removing the furin cleavage site required for activation and which contains the ΔH69, ΔV70, ΔY144, N501Y, A570D, D614G, P681H, T716I, S982A and D1118H mutations (UK variant+D614G). Accordingly, in certain embodiments, an optimized nucleotide sequence of the invention may encode a prefusion stabilized SARS-COV-2 S protein, which has an amino acid sequence comprising SEQ ID NO: 155. In one embodiment, the optimized nucleotide sequence has the sequence of SEQ ID NO: 154. In other embodiments, the optimized nucleotide sequence is at least 60%, 65%, 70%, 75%, 80%, 85%, 90%, 95%, 96%, 97%, 98%, or 99% identical to SEQ ID NO: 154 and encodes the amino acid sequence of SEQ ID NO: 155.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) by removing the furin cleavage site required for activation, by mutating residues 986 and 987 to proline and which contains the ΔH69, ΔV70, ΔY144, N501Y, A570D, D614G, P681H, T716I, S982A and D1118H mutations (UK variant+D614G). Accordingly, in certain embodiments, an optimized nucleotide sequence of the invention may encode a prefusion stabilized SARS-COV-2 S protein, which has an amino acid sequence comprising SEQ ID NO: 157. In one embodiment, the optimized nucleotide sequence has the sequence of SEQ ID NO: 156. In other embodiments, the optimized nucleotide sequence is at least 60%, 65%, 70%, 75%, 80%, 85%, 90%, 95%, 96%, 97%, 98%, or 99% identical to SEQ ID NO: 156 and encodes the amino acid sequence of SEQ ID NO: 157.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) by removing the furin cleavage site required for activation, by mutating residues 817, 892, 899 and 942, 986 and 987 to proline and which contains the H69, ΔV70, ΔΥ144, N501Y, A570D, D614G, P681H, T716I, S982A and D1118H mutations (UK variant+D614G). Accordingly, in certain embodiments, an optimized nucleotide sequence of the invention may encode a prefusion stabilized SARS-COV-2 S protein, which has an amino acid sequence comprising SEQ ID NO: 159. In one embodiment, the optimized nucleotide sequence has the sequence of SEQ ID NO: 158. In other embodiments, the optimized nucleotide sequence is at least 60%, 65%, 70%, 75%, 80%, 85%, 90%, 95%, 96%, 97%, 98%, or 99% identical to SEQ ID NO: 158 and encodes the amino acid sequence of SEQ ID NO: 159.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) to contain the D80A, D215G, ΔL242, ΔA243, ΔL244, K417N, E484K, N501Y, D614G and A701V mutations (South African variant 1+D614G). Accordingly, in certain embodiments, an optimized nucleotide sequence of the invention may encode a prefusion stabilized SARS-COV-2 S protein, which has an amino acid sequence comprising SEQ ID NO: 161. In one embodiment, the optimized nucleotide sequence has the sequence of SEQ ID NO: 160. In other embodiments, the optimized nucleotide sequence is at least 60%, 65%, 70%, 75%, 80%, 85%, 90%, 95%, 96%, 97%, 98%, or 99% identical to SEQ ID NO: 160 and encodes the amino acid sequence of SEQ ID NO: 161.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) by removing the furin cleavage site required for activation, by mutating residues 986 and 987 to proline and which contains the D80A, D215G, ΔL242, ΔA243, ΔL244, K417N, E484K, N501Y, D614G and A701V mutations (South African variant 1+D614G). Accordingly, in certain embodiments, an optimized nucleotide sequence of the invention may encode a prefusion stabilized SARS-COV-2 S protein, which has an amino acid sequence comprising SEQ ID NO: 163. In one embodiment, the optimized nucleotide sequence has the sequence of SEQ ID NO: 162. In other embodiments, the optimized nucleotide sequence is at least 60%, 65%, 70%, 75%, 80%, 85%, 90%, 95%, 96%, 97%, 98%, or 99% identical to SEQ ID NO: 162 and encodes the amino acid sequence of SEQ ID NO: 163.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) to contain the L18F, D80A, D215G, ΔL242, ΔA243, ΔL244, K417N, E484K, N501Y, D614G and A701V mutations (South African variant 2+D614G). Accordingly, in certain embodiments, an optimized nucleotide sequence of the invention may encode a prefusion stabilized SARS-COV-2 S protein, which has an amino acid sequence comprising SEQ ID NO: 165. In one embodiment, the optimized nucleotide sequence has the sequence of SEQ ID NO: 164. In other embodiments, the optimized nucleotide sequence is at least 60%, 65%, 70%, 75%, 80%, 85%, 90%, 95%, 96%, 97%, 98%, or 99% identical to SEQ ID NO: 164 and encodes the amino acid sequence of SEQ ID NO: 165.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) by removing the furin cleavage site required for activation, by mutating residues 986 and 987 to proline and which contains the L18F, D80A, D215G, ΔL242, ΔA243, ΔL244, K417N, E484K, N501Y, D614G and A701V mutations (South African variant 2+D614G). Accordingly, in certain embodiments, an optimized nucleotide sequence of the invention may encode a prefusion stabilized SARS-COV-2 S protein, which has an amino acid sequence comprising SEQ ID NO: 167. In one embodiment, the optimized nucleotide sequence has the sequence of SEQ ID NO: 166. In other embodiments, the optimized nucleotide sequence is at least 60%, 65%, 70%, 75%, 80%, 85%, 90%, 95%, 96%, 97%, 98%, or 99% identical to SEQ ID NO: 166 and encodes the amino acid sequence of SEQ ID NO: 167.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) to contain the L18F, T20N, P26S, D138Y, R190S, K417T, E484K, N501Y, D614G, H655Y, T1027I and V1176F mutations (Brazilian variant+D614G). Accordingly, in certain embodiments, an optimized nucleotide sequence of the invention may encode a prefusion stabilized SARS-COV-2 S protein, which has an amino acid sequence comprising SEQ ID NO: 169. In one embodiment, the optimized nucleotide sequence has the sequence of SEQ ID NO: 168. In other embodiments, the optimized nucleotide sequence is at least 60%, 65%, 70%, 75%, 80%, 85%, 90%, 95%, 96%, 97%, 98%, or 99% identical to SEQ ID NO: 168 and encodes the amino acid sequence of SEQ ID NO: 169.

In some embodiments, an optimized nucleotide sequence according to the present invention may encode a prefusion stabilized SARS-COV-2 S protein, a prefusion stabilized ectodomain of the SARS-COV-2 S protein, or an antigenic fragment of either, which has been modified relative to SARS-COV-2 S protein of the index strain from Wuhan (SEQ ID NO: 1) by removing the furin cleavage site required for activation, by mutating residues 986 and 987 to proline and which contains L18F, T20N, P26S, D138Y, R190S, K417T, E484K, N501Y, D614G, H655Y, T1027I and V1176F mutations (Brazilian variant+D614G). Accordingly, in certain embodiments, an optimized nucleotide sequence of the invention may encode a prefusion stabilized SARS-COV-2 S protein, which has an amino acid sequence comprising SEQ ID NO: 171. In one embodiment, the optimized nucleotide sequence has the sequence of SEQ ID NO: 170. In other embodiments, the optimized nucleotide sequence is at least 60%, 65%, 70%, 75%, 80%, 85%, 90%, 95%, 96%, 97%, 98%, or 99% identical to SEQ ID NO: 170 and encodes the amino acid sequence of SEQ ID NO: 171.

### Exemplary Optimized Nucleotide Sequences Encoding a SARS-Cov-2 S Protein and Antigenic Fragments

An optimized nucleotide sequence according to the present invention may encode a SARS-COV-2 S protein or an antigenic fragment thereof. In one embodiment, the invention provides a nucleic acid comprising an optimized nucleotide sequence encoding a SARS-COV-2 S protein or an antigenic fragment thereof. In some embodiments, the nucleic acid is an mRNA comprising an optimized nucleotide sequence encoding a SARS-COV-2 S protein or an antigenic fragment thereof. In some embodiments, a suitable mRNA sequence comprises a nucleotide sequence encoding a SARS-COV-2 S protein or an antigenic fragment thereof optimized for efficient expression human cells. Exemplary optimized nucleotide sequences encoding a SARS-CoV-2 S protein or an antigenic fragment thereof produced with the process for generating optimized nucleotide sequences in accordance with the invention and their corresponding amino acid sequence are shown in Table 1. Bold residues indicate those amino acids which have been mutated compared to a naturally occurring SARS-COV-2 S protein, underlined residues represent a signal peptide and the residues in italics indicate the presence of an Fc region or a Foldon.

TABLE 1
Exemplary SARS-CoV-2 S sequences.

| | |
|---|---|
| Optimized nucleotide | (SEQ ID NO: 29) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| | CACCAACTCCTTCACCAGAGGCGTGTATTACCCAGACAAGG |
| | TGTTTAGAAGCAGCGTGCTGCACTCTACCCAGGACCTCTTT |
| | CTGCCCTTTTTCAGCAACGTGACATGGTTTCACGCAATTCA |
| | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTAGAAGGGCCAGGTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCTT |
| | CATCGAGGATCTCCTGTTTAACAAGGTGACCCTGGCAGACG |
| | CCGGCTTTATTAAGCAATATGGGGATTGCCTGGGCGACATT |
| | GCTGCCAGAGACCTGATTTGCGCCCAGAAATTCAATGGCCT |
| | CACAGTGCTGCCACCTCTGCTGACCGACGAGATGATCGCTC |
| | AATACACTAGCGCACTGCTGGCCGGAACCATCACATCAGG |
| | CTGGACCTTCGGGGCCGGAGCAGCACTGCAGATTCCATTCG |
| | CCATGCAGATGGCCTATAGATTCAACGGCATTGGCGTCACA |
| | CAGAACGTGCTGTACGAAAACCAGAAGCTCATCGCTAACC |
| | AGTTTAATTCCGCAATTGGAAAGATCCAAGATTCACTCAGC |
| | TCAACCGCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGGATAAGGTGGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGTATATC |
| | AAATGGCCCTGGTACATTTGGCTGGGGTTTATCGCCGGACT |
| | GATTGCCATCGTCATGGTGACCATCATGCTGTGTTGCATGA |
| | CCTCCTGTTGTTCCTGTCTGAAGGGCTGCTGTAGTTGCGGCT |
| | CTTGCTGTAAATTCGACGAAGATGATAGCGAGCCCGTGCTG |
| | AAGGGCGTGAAGCTGCATTATACCTGA |
| SARS-CoV-2 S protein | (SEQ ID NO: 1) |
| sequence | MFVFLVLLPLVSSQCVNLTTRTQLPPAYTNSFTRGVYYPDKVF |
| | RSSVLHSTQDLFLPFFSNVTWFHAIHVSGTNGTKRFDNPVLPF |
| | NDGVYFASTEKSNIIRGWIFGTTLDSKTQSLLIVNNATNVVIKV |
| | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPRRARSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDKVEAEV |
| | QIDRLITGRLQSLQTYVTQQLIRAAEIRASANLAATKMSECVL |
| | GQSKRVDFCGKGYHLMSFPQSAPHGVVFLHVTYVPAQEKNF |
| | TTAPAICHDGKAHFPREGVFVSNGTHWFVTQRNFYEPQIITTD |
| | NTFVSGNCDVVIGIVNNTVYDPLQPELDSFKEELDKYFKNHTS |
| | PDVDLGDISGINASVVNIQKEIDRLNEVAKNLNESLIDLQELGK |
| | YEQYIKWPWYIWLGFIAGLIAIVMVTIMLCCMTSCCSCLKGCC |
| | SCGSCCKFDEDDSEPVLKGVKLHYT |
| Optimized nucleotide | (SEQ ID NO: 30) |
| sequence encoding the | ATGTTCGTCTTCCTCGTGCTGCTCCCACTCGTTTCTTCCCAG |
| ectodomain of a SARS- | TGTGTCAACCTGACAACTAGGACTCAGCTGCCACCAGCCTA |
| CoV-2 S protein | CACCAACTCCTTCACCAGAGGCGTGTATTACCCAGACAAGG |
| | TGTTTAGAAGCAGCGTGCTGCACTCTACCCAGGACCTCTTT |
| | CTGCCCTTTTTCAGCAACGTGACATGGTTTCACGCAATTCA |
| | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTAGAAGGGCCAGGTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCTT |
| | CATCGAGGATCTCCTGTTTAACAAGGTGACCCTGGCAGACG |
| | CCGGCTTTATTAAGCAATATGGGGATTGCCTGGGCGACATT |
| | GCTGCCAGAGACCTGATTTGCGCCCAGAAATTCAATGGCCT |
| | CACAGTGCTGCCACCTCTGCTGACCGACGAGATGATCGCTC |
| | AATACACTAGCGCACTGCTGGCCGGAACCATCACATCAGG |
| | CTGGACCTTCGGGGCCGGAGCAGCACTGCAGATTCCATTCG |
| | CCATGCAGATGGCCTATAGATTCAACGGCATTGGCGTCACA |
| | CAGAACGTGCTGTACGAAAACCAGAAGCTCATCGCTAACC |
| | AGTTTAATTCCGCAATTGGAAAGATCCAAGATTCACTCAGC |
| | TCAACCGCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGGATAAGGTGGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGTGA |
| Ectodomain of a SARS- | (SEQ ID NO: 2) |
| CoV-2 S protein | MFVFLVLLPLVSSQCVNLTTRTQLPPAYTNSFTRGVYYPDKVF |
| | RSSVLHSTQDLFLPFFSNVTWFHAIHVSGTNGTKRFDNPVLPF |
| | NDGVYFASTEKSNIIRGWIFGTTLDSKTQSLLIVNNATNVVIKV |
| | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPRRARSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDKVEAEV |
| | QIDRLITGRLQSLQTYVTQQLIRAAEIRASANLAATKMSECVL |
| | GQSKRVDFCGKGYHLMSFPQSAPHGVVFLHVTYVPAQEKNF |
| | TTAPAICHDGKAHFPREGVFVSNGTHWFVTQRNFYEPQIITTD |
| | NTFVSGNCDVVIGIVNNTVYDPLQPELDSFKEELDKYFKNHTS |
| | PDVDLGDISGINASVVNIQKEIDRLNEVAKNLNESLIDLQELGK |
| | YEQ |
| Optimized nucleotide | (SEQ ID NO: 31) |
| sequence encoding the | ATGTTCGTCTTCCTCGTGCTGCTCCCACTCGTTTCTTCCCAG |
| S1 subunit of a SARS- | TGTGTCAACCTGACAACTAGGACTCAGCTGCCACCAGCCTA |
| CoV-2 S protein | CACCAACTCCTTCACCAGAGGCGTGTATTACCCAGACAAGG |
| | TGTTTAGAAGCAGCGTGCTGCACTCTACCCAGGACCTCTTT |
| | CTGCCCTTTTTCAGCAACGTGACATGGTTTCACGCAATTCA |
| | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTTGA |
| S1 subunit of a SARS- | (SEQ ID NO: 3) |
| CoV-2 S protein | MFVFLVLLPLVSSQCVNLTTRTQLPPAYTNSFTRGVYYPDKVF |
| | RSSVLHSTQDLFLPFFSNVTWFHAIHVSGTNGTKRFDNPVLPF |
| | NDGVYFASTEKSNIIRGWIFGTTLDSKTQSLLIVNNATNVVIKV |
| | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSP |
| Optimized nucleotide | (SEQ ID NO: 32) |
| sequence encoding the | ATGTCCGTTGCTAGTCAGTCTATTATTGCCTATACCATGAG |
| subunit of a SARS-CoV- | CCTCGGAGCTGAGAATAGCGTGGCCTACTCCAATAATTCCA |
| 2 S protein | TCGCAATCCCTACTAACTTCACTATTTCTGTGACCACCGAG |
| | ATCCTGCCTGTGTCTATGACTAAGACTAGCGTTGATTGTAC |
| | CATGTATATTTGTGGCGACTCTACCGAATGTTCTAACCTGC |
| | TGCTTCAGTACGGCTCATTTTGCACACAGCTGAACAGAGCC |
| | CTGACTGGGATCGCTGTGGAGCAGGACAAGAACACACAGG |
| | AGGTGTTTGCACAGGTGAAGCAGATCTATAAGACCCCTCCT |
| | ATTAAGGATTTCGGCGGATTCAATTTCTCACAGATTCTGCC |
| | AGACCCCAGTAAGCCTTCCAAGAGGAGCTTCATCGAGGAT |
| | CTCCTGTTTAACAAGGTGACCCTGGCAGACGCCGGCTTTAT |
| | TAAGCAATATGGGGATTGCCTGGGCGACATTGCTGCCAGA |
| | GACCTGATTTGCGCCCAGAAATTCAATGGCCTCACAGTGCT |
| | GCCACCTCTGCTGACCGACGAGATGATCGCTCAATACACTA |
| | GCGCACTGCTGGCCGGAACCATCACATCAGGCTGGACCTTC |
| | GGGGCCGGAGCAGCACTGCAGATTCCATTCGCCATGCAGA |
| | TGGCCTATAGATTCAACGGCATTGGCGTCACACAGAACGTG |
| | CTGTACGAAAACCAGAAGCTCATCGCTAACCAGTTTAATTC |
| | CGCAATTGGAAAGATCCAAGATTCACTCAGCTCAACCGCCT |
| | CTGCACTCGGAAAGCTGCAGGACGTGGTCAACCAGAATGC |
| | TCAGGCCCTGAACACACTCGTCAAGCAGCTGTCCTCTAACT |
| | TTGGCGCTATCAGCTCCGTTCTGAACGACATTCTGAGCCGC |
| | CTGGATAAGGTGGAGGCTGAAGTCCAGATTGACCGCCTGA |
| | TTACCGGCCGGCTGCAGTCTCTGCAAACATACGTGACCCAG |
| | CAGCTGATCAGAGCAGCCGAGATCCGGGCATCCGCAAATC |
| | TGGCAGCAACTAAGATGAGCGAATGCGTGCTGGGCCAGTC |
| | CAAGCGGGTGGACTTTTGTGGCAAGGGCTACCACCTGATG |
| | AGCTTCCCCCAGAGCGCCCCACATGGCGTTGTTTTTCTGCA |
| | CGTGACCTATGTCCCTGCTCAGGAAAAGAACTTTACAACTG |
| | CTCCTGCTATCTGCCATGACGGCAAGGCCCACTTCCCACGG |
| | GAGGGAGTGTTTGTGTCCAATGGCACACACTGGTTCGTGAC |
| | CCAGAGGAACTTCTATGAACCCCAGATCATCACCACTGACA |
| | ATACCTTCGTGTCTGGAAATTGCGACGTCGTGATCGGCATC |
| | GTTAACAACACCGTGTACGACCCTCTCCAGCCAGAGCTGGA |
| | CTCCTTTAAGGAGGAACTGGATAAGTATTTTAAGAACCACA |
| | CAAGCCCAGATGTGGATCTCGGGGACATCTCCGGAATTAA |
| | CGCCTCCGTGGTGAATATCCAGAAGGAGATTGACCGCCTA |
| | AATGAAGTTGCCAAGAACCTCAATGAGTCTCTGATTGATCT |
| | GCAGGAACTGGGCAAGTATGAGCAGTGA |
| S2 subunit of a SARS- | (SEQ ID NO: 4) |
| CoV-2 S protein | MSVASQSIIAYTMSLGAENSVAYSNNSIAIPTNFTISVTTEILPV |
| | SMTKTSVDCTMYICGDSTECSNLLLQYGSFCTQLNRALTGIAV |
| | EQDKNTQEVFAQVKQIYKTPPIKDFGGFNFSQILPDPSKPSKRS |
| | FIEDLLFNKVTLADAGFIKQYGDCLGDIAARDLICAQKFNGLT |
| | VLPPLLTDEMIAQYTSALLAGTITSGWTFGAGAALQIPFAMQ |
| | MAYRFNGIGVTQNVLYENQKLIANQFNSAIGKIQDSLSSTASA |
| | LGKLQDVVNQNAQALNTLVKQLSSNFGAISSVLNDILSRLDK |
| | VEAEVQIDRLITGRLQSLQTYVTQQLIRAAEIRASANLAATKM |
| | SECVLGQSKRVDFCGKGYHLMSFPQSAPHGVVFLHVTYVPA |
| | QEKNFTTAPAICHDGKAHFPREGVFVSNGTHWFVTQRNFYEP |
| | QIITTDNTFVSGNCDVVIGIVNNTVYDPLQPELDSFKEELDKYF |
| | KNHTSPDVDLGDISGINASVVNIQKEIDRLNEVAKNLNESLIDL |
| | QELGKYEQ |
| Optimized nucleotide | (SEQ ID NO: 71) |
| sequence encoding the | ATGTCCGTTGCTAGTCAGTCTATTATTGCCTATACCATGAG |
| full length S2 subunit of | CCTCGGAGCTGAGAATAGCGTGGCCTACTCCAATAATTCCA |
| a SARS-CoV-2 S protein | TCGCAATCCCTACTAACTTCACTATTTCTGTGACCACCGAG |
| | ATCCTGCCTGTGTCTATGACTAAGACTAGCGTTGATTGTAC |
| | CATGTATATTTGTGGCGACTCTACCGAATGTTCTAACCTGC |
| | TGCTTCAGTACGGCTCATTTTGCACACAGCTGAACAGAGCC |
| | CTGACTGGGATCGCTGTGGAGCAGGACAAGAACACACAGG |
| | AGGTGTTTGCACAGGTGAAGCAGATCTATAAGACCCCTCCT |
| | ATTAAGGATTTCGGCGGATTCAATTTCTCACAGATTCTGCC |
| | AGACCCCAGTAAGCCTTCCAAGAGGAGCTTCATCGAGGAT |
| | CTCCTGTTTAACAAGGTGACCCTGGCAGACGCCGGCTTTAT |
| | TAAGCAATATGGGGATTGCCTGGGCGACATTGCTGCCAGA |
| | GACCTGATTTGCGCCCAGAAATTCAATGGCCTCACAGTGCT |
| | GCCACCTCTGCTGACCGACGAGATGATCGCTCAATACACTA |
| | GCGCACTGCTGGCCGGAACCATCACATCAGGCTGGACCTTC |
| | GGGGCCGGAGCAGCACTGCAGATTCCATTCGCCATGCAGA |
| | TGGCCTATAGATTCAACGGCATTGGCGTCACACAGAACGTG |
| | CTGTACGAAAACCAGAAGCTCATCGCTAACCAGTTTAATTC |
| | CGCAATTGGAAAGATCCAAGATTCACTCAGCTCAACCGCCT |
| | CTGCACTCGGAAAGCTGCAGGACGTGGTCAACCAGAATGC |
| | TCAGGCCCTGAACACACTCGTCAAGCAGCTGTCCTCTAACT |
| | TTGGCGCTATCAGCTCCGTTCTGAACGACATTCTGAGCCGC |
| | CTGGATAAGGTGGAGGCTGAAGTCCAGATTGACCGCCTGA |
| | TTACCGGCCGGCTGCAGTCTCTGCAAACATACGTGACCCAG |
| | CAGCTGATCAGAGCAGCCGAGATCCGGGCATCCGCAAATC |
| | TGGCAGCAACTAAGATGAGCGAATGCGTGCTGGGCCAGTC |
| | CAAGCGGGTGGACTTTTGTGGCAAGGGCTACCACCTGATG |
| | AGCTTCCCCCAGAGCGCCCCACATGGCGTTGTTTTTCTGCA |
| | CGTGACCTATGTCCCTGCTCAGGAAAAGAACTTTACAACTG |
| | CTCCTGCTATCTGCCATGACGGCAAGGCCCACTTCCCACGG |
| | GAGGGAGTGTTTGTGTCCAATGGCACACACTGGTTCGTGAC |
| | CCAGAGGAACTTCTATGAACCCCAGATCATCACCACTGACA |
| | ATACCTTCGTGTCTGGAAATTGCGACGTCGTGATCGGCATC |
| | GTTAACAACACCGTGTACGACCCTCTCCAGCCAGAGCTGGA |
| | CTCCTTTAAGGAGGAACTGGATAAGTATTTTAAGAACCACA |
| | CAAGCCCAGATGTGGATCTCGGGGACATCTCCGGAATTAA |
| | CGCCTCCGTGGTGAATATCCAGAAGGAGATTGACCGCCTA |
| | AATGAAGTTGCCAAGAACCTCAATGAGTCTCTGATTGATCT |
| | GCAGGAACTGGGCAAGTATGAGCAGTATATCAAATGGCCC |
| | TGGTACATTTGGCTGGGGTTTATCGCCGGACTGATTGCCAT |
| | CGTCATGGTGACCATCATGCTGTGTTGCATGACCTCCTGTT |
| | GTTCCTGTCTGAAGGGCTGCTGTAGTTGCGGCTCTTGCTGT |
| | AAATTCGACGAAGATGATAGCGAGCCCGTGCTGAAGGGCG |
| | TGAAGCTGCATTATACCTGA |
| Full length S2 subunit of | (SEQ ID NO: 72) |
| a SARS-CoV-2 S protein | MSVASQSIIAYTMSLGAENSVAYSNNSIAIPTNFTISVTTEILPV |
| | SMTKTSVDCTMYICGDSTECSNLLLQYGSFCTQLNRALTGIAV |
| | EQDKNTQEVFAQVKQIYKTPPIKDFGGFNFSQILPDPSKPSKRS |
| | FIEDLLFNKVTLADAGFIKQYGDCLGDIAARDLICAQKFNGLT |
| | VLPPLLTDEMIAQYTSALLAGTITSGWTFGAGAALQIPFAMQ |
| | MAYRFNGIGVTQNVLYENQKLIANQFNSAIGKIQDSLSSTASA |
| | LGKLQDVVNQNAQALNTLVKQLSSNFGAISSVLNDILSRLDK |
| | VEAEVQIDRLITGRLQSLQTYVTQQLIRAAEIRASANLAATKM |
| | SECVLGQSKRVDFCGKGYHLMSFPQSAPHGVVFLHVTYVPA |
| | QEKNFTTAPAICHDGKAHFPREGVFVSNGTHWFVTQRNFYEP |
| | QIITTDNTFVSGNCDVVIGIVNNTVYDPLQPELDSFKEELDKYF |
| | KNHTSPDVDLGDISGINASVVNIQKEIDRLNEVAKNLNESLIDL |
| | QELGKYEQYIKWPWYIWLGFIAGLIAIVMVTIMLCCMTSCCSC |
| | LKGCCSCGSCCKFDEDDSEPVLKGVKLHYT |
| Optimized nucleotide | (SEQ ID NO: 73) |
| sequence encoding the | ATGTTCGTCTTCCTCGTGCTGCTCCCACTCGTTTCTTCCCAG |
| S2 subunit of a SARS- | TGTTCCGTTGCTAGTCAGTCTATTATTGCCTATACCATGAGC |
| CoV-2 S protein with a | CTCGGAGCTGAGAATAGCGTGGCCTACTCCAATAATTCCAT |
| signal sequence | CGCAATCCCTACTAACTTCACTATTTCTGTGACCACCGAGA |
| | TCCTGCCTGTGTCTATGACTAAGACTAGCGTTGATTGTACC |
| | ATGTATATTTGTGGCGACTCTACCGAATGTTCTAACCTGCT |
| | GCTTCAGTACGGCTCATTTTGCACACAGCTGAACAGAGCCC |
| | TGACTGGGATCGCTGTGGAGCAGGACAAGAACACACAGGA |
| | GGTGTTTGCACAGGTGAAGCAGATCTATAAGACCCCTCCTA |
| | TTAAGGATTTCGGCGGATTCAATTTCTCACAGATTCTGCCA |
| | GACCCCAGTAAGCCTTCCAAGAGGAGCTTCATCGAGGATCT |
| | CCTGTTTAACAAGGTGACCCTGGCAGACGCCGGCTTTATTA |
| | AGCAATATGGGGATTGCCTGGGCGACATTGCTGCCAGAGA |
| | CCTGATTTGCGCCCAGAAATTCAATGGCCTCACAGTGCTGC |
| | CACCTCTGCTGACCGACGAGATGATCGCTCAATACACTAGC |
| | GCACTGCTGGCCGGAACCATCACATCAGGCTGGACCTTCGG |
| | GGCCGGAGCAGCACTGCAGATTCCATTCGCCATGCAGATG |
| | GCCTATAGATTCAACGGCATTGGCGTCACACAGAACGTGCT |
| | GTACGAAAACCAGAAGCTCATCGCTAACCAGTTTAATTCCG |
| | CAATTGGAAAGATCCAAGATTCACTCAGCTCAACCGCCTCT |
| | GCACTCGGAAAGCTGCAGGACGTGGTCAACCAGAATGCTC |
| | AGGCCCTGAACACACTCGTCAAGCAGCTGTCCTCTAACTTT |
| | GGCGCTATCAGCTCCGTTCTGAACGACATTCTGAGCCGCCT |
| | GGATAAGGTGGAGGCTGAAGTCCAGATTGACCGCCTGATT |
| | ACCGGCCGGCTGCAGTCTCTGCAAACATACGTGACCCAGC |
| | AGCTGATCAGAGCAGCCGAGATCCGGGCATCCGCAAATCT |
| | GGCAGCAACTAAGATGAGCGAATGCGTGCTGGGCCAGTCC |
| | AAGCGGGTGGACTTTTGTGGCAAGGGCTACCACCTGATGA |
| | GCTTCCCCCAGAGCGCCCCACATGGCGTTGTTTTTCTGCAC |
| | GTGACCTATGTCCCTGCTCAGGAAAAGAACTTTACAACTGC |
| | TCCTGCTATCTGCCATGACGGCAAGGCCCACTTCCCACGGG |
| | AGGGAGTGTTTGTGTCCAATGGCACACACTGGTTCGTGACC |
| | CAGAGGAACTTCTATGAACCCCAGATCATCACCACTGACA |
| | ATACCTTCGTGTCTGGAAATTGCGACGTCGTGATCGGCATC |
| | GTTAACAACACCGTGTACGACCCTCTCCAGCCAGAGCTGGA |
| | CTCCTTTAAGGAGGAACTGGATAAGTATTTTAAGAACCACA |
| | CAAGCCCAGATGTGGATCTCGGGGACATCTCCGGAATTAA |
| | CGCCTCCGTGGTGAATATCCAGAAGGAGATTGACCGCCTA |
| | AATGAAGTTGCCAAGAACCTCAATGAGTCTCTGATTGATCT |
| | GCAGGAACTGGGCAAGTATGAGCAGTGA |
| S2 subunit of a SARS- | (SEQ ID NO: 74) |
| CoV-2 S protein with a | MFVFLVLLPLVSSQCSVASQSIIAYTMSLGAENSVAYSNNSIAI |
| signal sequence | PTNFTISVTTEILPVSMTKTSVDCTMYICGDSTECSNLLLQYGS |
| | FCTQLNRALTGIAVEQDKNTQEVFAQVKQIYKTPPIKDFGGFN |
| | FSQILPDPSKPSKRSFIEDLLFNKVTLADAGFIKQYGDCLGDIA |
| | ARDLICAQKFNGLTVLPPLLTDEMIAQYTSALLAGTITSGWTF |
| | GAGAALQIPFAMQMAYRFNGIGVTQNVLYENQKLIANQFNSA |
| | IGKIQDSLSSTASALGKLQDVVNQNAQALNTLVKQLSSNFGAI |
| | SSVLNDILSRLDKVEAEVQIDRLITGRLQSLQTYVTQQLIRAAE |
| | IRASANLAATKMSECVLGQSKRVDFCGKGYHLMSFPQSAPHG |
| | VVFLHVTYVPAQEKNFTTAPAICHDGKAHFPREGVFVSNGTH |
| | WFVTQRNFYEPQIITTDNTFVSGNCDVVIGIVNNTVYDPLQPE |
| | LDSFKEELDKYFKNHTSPDVDLGDISGINASVVNIQKEIDRLNE |
| | VAKNLNESLIDLQELGKYEQ |
| Optimized nucleotide | (SEQ ID NO: 67) |
| sequence encoding the | ATGTTCGTCTTCCTCGTGCTGCTCCCACTCGTTTCTTCCCAG |
| full length S2 subunit of | TGTTCCGTTGCTAGTCAGTCTATTATTGCCTATACCATGAGC |
| a SARS-CoV-2 S protein | CTCGGAGCTGAGAATAGCGTGGCCTACTCCAATAATTCCAT |
| with a signal sequence | CGCAATCCCTACTAACTTCACTATTTCTGTGACCACCGAGA |
| | TCCTGCCTGTGTCTATGACTAAGACTAGCGTTGATTGTACC |
| | ATGTATATTTGTGGCGACTCTACCGAATGTTCTAACCTGCT |
| | GCTTCAGTACGGCTCATTTTGCACACAGCTGAACAGAGCCC |
| | TGACTGGGATCGCTGTGGAGCAGGACAAGAACACACAGGA |
| | GGTGTTTGCACAGGTGAAGCAGATCTATAAGACCCCTCCTA |
| | TTAAGGATTTCGGCGGATTCAATTTCTCACAGATTCTGCCA |
| | GACCCCAGTAAGCCTTCCAAGAGGAGCTTCATCGAGGATCT |
| | CCTGTTTAACAAGGTGACCCTGGCAGACGCCGGCTTTATTA |
| | AGCAATATGGGGATTGCCTGGGCGACATTGCTGCCAGAGA |
| | CCTGATTTGCGCCCAGAAATTCAATGGCCTCACAGTGCTGC |
| | CACCTCTGCTGACCGACGAGATGATCGCTCAATACACTAGC |
| | GCACTGCTGGCCGGAACCATCACATCAGGCTGGACCTTCGG |
| | GGCCGGAGCAGCACTGCAGATTCCATTCGCCATGCAGATG |
| | GCCTATAGATTCAACGGCATTGGCGTCACACAGAACGTGCT |
| | GTACGAAAACCAGAAGCTCATCGCTAACCAGTTTAATTCCG |
| | CAATTGGAAAGATCCAAGATTCACTCAGCTCAACCGCCTCT |
| | GCACTCGGAAAGCTGCAGGACGTGGTCAACCAGAATGCTC |
| | AGGCCCTGAACACACTCGTCAAGCAGCTGTCCTCTAACTTT |
| | GGCGCTATCAGCTCCGTTCTGAACGACATTCTGAGCCGCCT |
| | GGATAAGGTGGAGGCTGAAGTCCAGATTGACCGCCTGATT |
| | ACCGGCCGGCTGCAGTCTCTGCAAACATACGTGACCCAGC |
| | AGCTGATCAGAGCAGCCGAGATCCGGGCATCCGCAAATCT |
| | GGCAGCAACTAAGATGAGCGAATGCGTGCTGGGCCAGTCC |
| | AAGCGGGTGGACTTTTGTGGCAAGGGCTACCACCTGATGA |
| | GCTTCCCCCAGAGCGCCCCACATGGCGTTGTTTTTCTGCAC |
| | GTGACCTATGTCCCTGCTCAGGAAAAGAACTTTACAACTGC |
| | TCCTGCTATCTGCCATGACGGCAAGGCCCACTTCCCACGGG |
| | AGGGAGTGTTTGTGTCCAATGGCACACACTGGTTCGTGACC |
| | CAGAGGAACTTCTATGAACCCCAGATCATCACCACTGACA |
| | ATACCTTCGTGTCTGGAAATTGCGACGTCGTGATCGGCATC |
| | GTTAACAACACCGTGTACGACCCTCTCCAGCCAGAGCTGGA |
| | CTCCTTTAAGGAGGAACTGGATAAGTATTTTAAGAACCACA |
| | CAAGCCCAGATGTGGATCTCGGGGACATCTCCGGAATTAA |
| | CGCCTCCGTGGTGAATATCCAGAAGGAGATTGACCGCCTA |
| | AATGAAGTTGCCAAGAACCTCAATGAGTCTCTGATTGATCT |
| | GCAGGAACTGGGCAAGTATGAGCAGTATATCAAATGGCCC |
| | TGGTACATTTGGCTGGGGTTTATCGCCGGACTGATTGCCAT |
| | CGTCATGGTGACCATCATGCTGTGTTGCATGACCTCCTGTT |
| | GTTCCTGTCTGAAGGGCTGCTGTAGTTGCGGCTCTTGCTGT |
| | AAATTCGACGAAGATGATAGCGAGCCCGTGCTGAAGGGCG |
| | TGAAGCTGCATTATACCTGA |
| Full length S2 subunit of | (SEQ ID NO: 68) |
| a SARS-CoV-2 S protein | MFVFLVLLPLVSSQCSVASQSIIAYTMSLGAENSVAYSNNSIAI |
| with a signal sequence | PTNFTISVTTEILPVSMTKTSVDCTMYICGDSTECSNLLLQYGS |
| | FCTQLNRALTGIAVEQDKNTQEVFAQVKQIYKTPPIKDFGGFN |
| | FSQILPDPSKPSKRSFIEDLLFNKVTLADAGFIKQYGDCLGDIA |
| | ARDLICAQKFNGLTVLPPLLTDEMIAQYTSALLAGTITSGWTF |
| | GAGAALQIPFAMQMAYRFNGIGVTQNVLYENQKLIANQFNSA |
| | IGKIQDSLSSTASALGKLQDVVNQNAQALNTLVKQLSSNFGAI |
| | SSVLNDILSRLDKVEAEVQIDRLITGRLQSLQTYVTQQLIRAAE |
| | IRASANLAATKMSECVLGQSKRVDFCGKGYHLMSFPQSAPHG |
| | VVFLHVTYVPAQEKNFTTAPAICHDGKAHFPREGVFVSNGTH |
| | WFVTQRNFYEPQIITTDNTFVSGNCDVVIGIVNNTVYDPLQPE |
| | LDSFKEELDKYFKNHTSPDVDLGDISGINASVVNIQKEIDRLNE |
| | VAKNLNESLIDLQELGKYEQYIKWPWYIWLGFIAGLIAIVMVT |
| | IMLCCMTSCCSCLKGCCSCGSCCKFDEDDSEPVLKGVKLHYT |
| Optimized nucleotide | (SEQ ID NO: 33) |
| sequence encoding the | ATGAGCTTCATCGAGGATCTCCTGTTTAACAAGGTGACCCT |
| S2′ subunit of a SARS- | GGCAGACGCCGGCTTTATTAAGCAATATGGGGATTGCCTGG |
| CoV-2 S protein | GCGACATTGCTGCCAGAGACCTGATTTGCGCCCAGAAATTC |
| | AATGGCCTCACAGTGCTGCCACCTCTGCTGACCGACGAGAT |
| | GATCGCTCAATACACTAGCGCACTGCTGGCCGGAACCATCA |
| | CATCAGGCTGGACCTTCGGGGCCGGAGCAGCACTGCAGAT |
| | TCCATTCGCCATGCAGATGGCCTATAGATTCAACGGCATTG |
| | GCGTCACACAGAACGTGCTGTACGAAAACCAGAAGCTCAT |
| | CGCTAACCAGTTTAATTCCGCAATTGGAAAGATCCAAGATT |
| | CACTCAGCTCAACCGCCTCTGCACTCGGAAAGCTGCAGGAC |
| | GTGGTCAACCAGAATGCTCAGGCCCTGAACACACTCGTCA |
| | AGCAGCTGTCCTCTAACTTTGGCGCTATCAGCTCCGTTCTG |
| | AACGACATTCTGAGCCGCCTGGATAAGGTGGAGGCTGAAG |
| | TCCAGATTGACCGCCTGATTACCGGCCGGCTGCAGTCTCTG |
| | CAAACATACGTGACCCAGCAGCTGATCAGAGCAGCCGAGA |
| | TCCGGGCATCCGCAAATCTGGCAGCAACTAAGATGAGCGA |
| | ATGCGTGCTGGGCCAGTCCAAGCGGGTGGACTTTTGTGGCA |
| | AGGGCTACCACCTGATGAGCTTCCCCCAGAGCGCCCCACAT |
| | GGCGTTGTTTTTCTGCACGTGACCTATGTCCCTGCTCAGGA |
| | AAAGAACTTTACAACTGCTCCTGCTATCTGCCATGACGGCA |
| | AGGCCCACTTCCCACGGGAGGGAGTGTTTGTGTCCAATGGC |
| | ACACACTGGTTCGTGACCCAGAGGAACTTCTATGAACCCCA |
| | GATCATCACCACTGACAATACCTTCGTGTCTGGAAATTGCG |
| | ACGTCGTGATCGGCATCGTTAACAACACCGTGTACGACCCT |
| | CTCCAGCCAGAGCTGGACTCCTTTAAGGAGGAACTGGATA |
| | AGTATTTTAAGAACCACACAAGCCCAGATGTGGATCTCGG |
| | GGACATCTCCGGAATTAACGCCTCCGTGGTGAATATCCAGA |
| | AGGAGATTGACCGCCTAAATGAAGTTGCCAAGAACCTCAA |
| | TGAGTCTCTGATTGATCTGCAGGAACTGGGCAAGTATGAGC |
| | AGTAA |
| S2′ subunit of a SARS- | (SEQ ID NO: 5) |
| CoV-2 S protein | MSFIEDLLFNKVTLADAGFIKQYGDCLGDIAARDLICAQKFNG |
| | LTVLPPLLTDEMIAQYTSALLAGTITSGWTFGAGAALQIPFAM |
| | QMAYRFNGIGVTQNVLYENQKLIANQFNSAIGKIQDSLSSTAS |
| | ALGKLQDVVNQNAQALNTLVKQLSSNFGAISSVLNDILSRLD |
| | KVEAEVQIDRLITGRLQSLQTYVTQQLIRAAEIRASANLAATK |
| | MSECVLGQSKRVDFCGKGYHLMSFPQSAPHGVVFLHVTYVP |
| | AQEKNFTTAPAICHDGKAHFPREGVFVSNGTHWFVTQRNFYE |
| | PQIITTDNTFVSGNCDVVIGIVNNTVYDPLQPELDSFKEELDKY |
| | FKNHTSPDVDLGDISGINASVVNIQKEIDRLNEVAKNLNESLID |
| | LQELGKYEQ |
| Optimized nucleotide | (SEQ ID NO: 97) |
| sequence encoding the | ATGAGCTTCATCGAGGATCTCCTGTTTAACAAGGTGACCCT |
| full length S2′ subunit of | GGCAGACGCCGGCTTTATTAAGCAATATGGGGATTGCCTGG |
| a SARS-CoV-2 S protein | GCGACATTGCTGCCAGAGACCTGATTTGCGCCCAGAAATTC |
| | AATGGCCTCACAGTGCTGCCACCTCTGCTGACCGACGAGAT |
| | GATCGCTCAATACACTAGCGCACTGCTGGCCGGAACCATCA |
| | CATCAGGCTGGACCTTCGGGGCCGGAGCAGCACTGCAGAT |
| | TCCATTCGCCATGCAGATGGCCTATAGATTCAACGGCATTG |
| | GCGTCACACAGAACGTGCTGTACGAAAACCAGAAGCTCAT |
| | CGCTAACCAGTTTAATTCCGCAATTGGAAAGATCCAAGATT |
| | CACTCAGCTCAACCGCCTCTGCACTCGGAAAGCTGCAGGAC |
| | GTGGTCAACCAGAATGCTCAGGCCCTGAACACACTCGTCA |
| | AGCAGCTGTCCTCTAACTTTGGCGCTATCAGCTCCGTTCTG |
| | AACGACATTCTGAGCCGCCTGGATAAGGTGGAGGCTGAAG |
| | TCCAGATTGACCGCCTGATTACCGGCCGGCTGCAGTCTCTG |
| | CAAACATACGTGACCCAGCAGCTGATCAGAGCAGCCGAGA |
| | TCCGGGCATCCGCAAATCTGGCAGCAACTAAGATGAGCGA |
| | ATGCGTGCTGGGCCAGTCCAAGCGGGTGGACTTTTGTGGCA |
| | AGGGCTACCACCTGATGAGCTTCCCCCAGAGCGCCCCACAT |
| | GGCGTTGTTTTTCTGCACGTGACCTATGTCCCTGCTCAGGA |
| | AAAGAACTTTACAACTGCTCCTGCTATCTGCCATGACGGCA |
| | AGGCCCACTTCCCACGGGAGGGAGTGTTTGTGTCCAATGGC |
| | ACACACTGGTTCGTGACCCAGAGGAACTTCTATGAACCCCA |
| | GATCATCACCACTGACAATACCTTCGTGTCTGGAAATTGCG |
| | ACGTCGTGATCGGCATCGTTAACAACACCGTGTACGACCCT |
| | CTCCAGCCAGAGCTGGACTCCTTTAAGGAGGAACTGGATA |
| | AGTATTTTAAGAACCACACAAGCCCAGATGTGGATCTCGG |
| | GGACATCTCCGGAATTAACGCCTCCGTGGTGAATATCCAGA |
| | AGGAGATTGACCGCCTAAATGAAGTTGCCAAGAACCTCAA |
| | TGAGTCTCTGATTGATCTGCAGGAACTGGGCAAGTATGAGC |
| | AGTATATCAAATGGCCCTGGTACATTTGGCTGGGGTTTATC |
| | GCCGGACTGATTGCCATCGTCATGGTGACCATCATGCTGTG |
| | TTGCATGACCTCCTGTTGTTCCTGTCTGAAGGGCTGCTGTA |
| | GTTGCGGCTCTTGCTGTAAATTCGACGAAGATGATAGCGAG |
| | CCCGTGCTGAAGGGCGTGAAGCTGCATTATACCTGA |
| Full length S2′ subunit | (SEQ ID NO: 98) |
| of a SARS-CoV-2 S | MSFIEDLLFNKVTLADAGFIKQYGDCLGDIAARDLICAQKFNG |
| protein | LTVLPPLLTDEMIAQYTSALLAGTITSGWTFGAGAALQIPFAM |
| | QMAYRFNGIGVTQNVLYENQKLIANQFNSAIGKIQDSLSSTAS |
| | ALGKLQDVVNQNAQALNTLVKQLSSNFGAISSVLNDILSRLD |
| | KVEAEVQIDRLITGRLQSLQTYVTQQLIRAAEIRASANLAATK |
| | MSECVLGQSKRVDFCGKGYHLMSFPQSAPHGVVFLHVTYVP |
| | AQEKNFTTAPAICHDGKAHFPREGVFVSNGTHWFVTQRNFYE |
| | PQIITTDNTFVSGNCDVVIGIVNNTVYDPLQPELDSFKEELDKY |
| | FKNHTSPDVDLGDISGINASVVNIQKEIDRLNEVAKNLNESLID |
| | LQELGKYEQYIKWPWYIWLGFIAGLIAIVMVTIMLCCMTSCCS |
| | CLKGCCSCGSCCKFDEDDSEPVLKGVKLHYT |
| Optimized nucleotide | (SEQ ID NO: 65) |
| sequence encoding the | ATGTTCGTCTTCCTCGTGCTGCTCCCACTCGTTTCTTCCCAG |
| S2′ subunit of a SARS- | TGTAGCTTCATCGAGGATCTCCTGTTTAACAAGGTGACCCT |
| CoV-2 S protein with a | GGCAGACGCCGGCTTTATTAAGCAATATGGGGATTGCCTGG |
| signal sequence | GCGACATTGCTGCCAGAGACCTGATTTGCGCCCAGAAATTC |
| | AATGGCCTCACAGTGCTGCCACCTCTGCTGACCGACGAGAT |
| | GATCGCTCAATACACTAGCGCACTGCTGGCCGGAACCATCA |
| | CATCAGGCTGGACCTTCGGGGCCGGAGCAGCACTGCAGAT |
| | TCCATTCGCCATGCAGATGGCCTATAGATTCAACGGCATTG |
| | GCGTCACACAGAACGTGCTGTACGAAAACCAGAAGCTCAT |
| | CGCTAACCAGTTTAATTCCGCAATTGGAAAGATCCAAGATT |
| | CACTCAGCTCAACCGCCTCTGCACTCGGAAAGCTGCAGGAC |
| | GTGGTCAACCAGAATGCTCAGGCCCTGAACACACTCGTCA |
| | AGCAGCTGTCCTCTAACTTTGGCGCTATCAGCTCCGTTCTG |
| | AACGACATTCTGAGCCGCCTGGATAAGGTGGAGGCTGAAG |
| | TCCAGATTGACCGCCTGATTACCGGCCGGCTGCAGTCTCTG |
| | CAAACATACGTGACCCAGCAGCTGATCAGAGCAGCCGAGA |
| | TCCGGGCATCCGCAAATCTGGCAGCAACTAAGATGAGCGA |
| | ATGCGTGCTGGGCCAGTCCAAGCGGGTGGACTTTTGTGGCA |
| | AGGGCTACCACCTGATGAGCTTCCCCCAGAGCGCCCCACAT |
| | GGCGTTGTTTTTCTGCACGTGACCTATGTCCCTGCTCAGGA |
| | AAAGAACTTTACAACTGCTCCTGCTATCTGCCATGACGGCA |
| | AGGCCCACTTCCCACGGGAGGGAGTGTTTGTGTCCAATGGC |
| | ACACACTGGTTCGTGACCCAGAGGAACTTCTATGAACCCCA |
| | GATCATCACCACTGACAATACCTTCGTGTCTGGAAATTGCG |
| | ACGTCGTGATCGGCATCGTTAACAACACCGTGTACGACCCT |
| | CTCCAGCCAGAGCTGGACTCCTTTAAGGAGGAACTGGATA |
| | AGTATTTTAAGAACCACACAAGCCCAGATGTGGATCTCGG |
| | GGACATCTCCGGAATTAACGCCTCCGTGGTGAATATCCAGA |
| | AGGAGATTGACCGCCTAAATGAAGTTGCCAAGAACCTCAA |
| | TGAGTCTCTGATTGATCTGCAGGAACTGGGCAAGTATGAGC |
| | AGTAA |
| S2′ subunit of a SARS- | (SEQ ID NO: 66) |
| CoV-2 S protein with a | MFVFLVLLPLVSSQCSFIEDLLFNKVTLADAGFIKQYGDCLGDI |
| signal sequence | AARDLICAQKFNGLTVLPPLLTDEMIAQYTSALLAGTITSGWT |
| | FGAGAALQIPFAMQMAYRFNGIGVTQNVLYENQKLIANQFNS |
| | AIGKIQDSLSSTASALGKLQDVVNQNAQALNTLVKQLSSNFG |
| | AISSVLNDILSRLDKVEAEVQIDRLITGRLQSLQTYVTQQLIRA |
| | AEIRASANLAATKMSECVLGQSKRVDFCGKGYHLMSFPQSAP |
| | HGVVFLHVTYVPAQEKNFTTAPAICHDGKAHFPREGVFVSNG |
| | THWFVTQRNFYEPQIITTDNTFVSGNCDVVIGIVNNTVYDPLQ |
| | PELDSFKEELDKYFKNHTSPDVDLGDISGINASVVNIQKEIDRL |
| | NEVAKNLNESLIDLQELGKYEQQCSFIEDLLFNKVTLADAGFI |
| | KQYGDCLGDIAARDLICAQKFNGLTVLPPLLTDEMIAQYTSAL |
| | LAGTITSGWTFGAGAALQIPFAMQMAYRFNGIGVTQNVLYEN |
| | QKLIANQFNSAIGKIQDSLSSTASALGKLQDVVNQNAQALNTL |
| | VKQLSSNFGAISSVLNDILSRLDKVEAEVQIDRLITGRLQSLQT |
| | YVTQQLIRAAEIRASANLAATKMSECVLGQSKRVDFCGKGYH |
| | LMSFPQSAPHGVVFLHVTYVPAQEKNFTTAPAICHDGKAHFP |
| | REGVFVSNGTHWFVTQRNFYEPQIITTDNTFVSGNCDVVIGIV |
| | NNTVYDPLQPELDSFKEELDKYFKNHTSPDVDLGDISGINASV |
| | VNIQKEIDRLNEVAKNLNESLIDLQELGKYEQ |
| Optimized nucleotide | (SEQ ID NO: 95) |
| sequence encoding the | ATGTTCGTCTTCCTCGTGCTGCTCCCACTCGTTTCTTCCCAG |
| full length S2′ subunit of | TGTAGCTTCATCGAGGATCTCCTGTTTAACAAGGTGACCCT |
| a SARS-CoV-2 S protein | GGCAGACGCCGGCTTTATTAAGCAATATGGGGATTGCCTGG |
| with a signal sequence | GCGACATTGCTGCCAGAGACCTGATTTGCGCCCAGAAATTC |
| | AATGGCCTCACAGTGCTGCCACCTCTGCTGACCGACGAGAT |
| | GATCGCTCAATACACTAGCGCACTGCTGGCCGGAACCATCA |
| | CATCAGGCTGGACCTTCGGGGCCGGAGCAGCACTGCAGAT |
| | TCCATTCGCCATGCAGATGGCCTATAGATTCAACGGCATTG |
| | GCGTCACACAGAACGTGCTGTACGAAAACCAGAAGCTCAT |
| | CGCTAACCAGTTTAATTCCGCAATTGGAAAGATCCAAGATT |
| | CACTCAGCTCAACCGCCTCTGCACTCGGAAAGCTGCAGGAC |
| | GTGGTCAACCAGAATGCTCAGGCCCTGAACACACTCGTCA |
| | AGCAGCTGTCCTCTAACTTTGGCGCTATCAGCTCCGTTCTG |
| | AACGACATTCTGAGCCGCCTGGATAAGGTGGAGGCTGAAG |
| | TCCAGATTGACCGCCTGATTACCGGCCGGCTGCAGTCTCTG |
| | CAAACATACGTGACCCAGCAGCTGATCAGAGCAGCCGAGA |
| | TCCGGGCATCCGCAAATCTGGCAGCAACTAAGATGAGCGA |
| | ATGCGTGCTGGGCCAGTCCAAGCGGGTGGACTTTTGTGGCA |
| | AGGGCTACCACCTGATGAGCTTCCCCCAGAGCGCCCCACAT |
| | GGCGTTGTTTTTCTGCACGTGACCTATGTCCCTGCTCAGGA |
| | AAAGAACTTTACAACTGCTCCTGCTATCTGCCATGACGGCA |
| | AGGCCCACTTCCCACGGGAGGGAGTGTTTGTGTCCAATGGC |
| | ACACACTGGTTCGTGACCCAGAGGAACTTCTATGAACCCCA |
| | GATCATCACCACTGACAATACCTTCGTGTCTGGAAATTGCG |
| | ACGTCGTGATCGGCATCGTTAACAACACCGTGTACGACCCT |
| | CTCCAGCCAGAGCTGGACTCCTTTAAGGAGGAACTGGATA |
| | AGTATTTTAAGAACCACACAAGCCCAGATGTGGATCTCGG |
| | GGACATCTCCGGAATTAACGCCTCCGTGGTGAATATCCAGA |
| | AGGAGATTGACCGCCTAAATGAAGTTGCCAAGAACCTCAA |
| | TGAGTCTCTGATTGATCTGCAGGAACTGGGCAAGTATGAGC |
| | AGTATATCAAATGGCCCTGGTACATTTGGCTGGGGTTTATC |
| | GCCGGACTGATTGCCATCGTCATGGTGACCATCATGCTGTG |
| | TTGCATGACCTCCTGTTGTTCCTGTCTGAAGGGCTGCTGTA |
| | GTTGCGGCTCTTGCTGTAAATTCGACGAAGATGATAGCGAG |
| | CCCGTGCTGAAGGGCGTGAAGCTGCATTATACCTGA |
| Full length S2′ subunit | (SEQ ID NO: 96) |
| of a SARS-CoV-2 S | MFVFLVLLPLVSSQCSFIEDLLFNKVTLADAGFIKQYGDCLGDI |
| protein with a signal | AARDLICAQKFNGLTVLPPLLTDEMIAQYTSALLAGTITSGWT |
| sequence | FGAGAALQIPFAMQMAYRFNGIGVTQNVLYENQKLIANQFNS |
| | AIGKIQDSLSSTASALGKLQDVVNQNAQALNTLVKQLSSNFG |
| | AISSVLNDILSRLDKVEAEVQIDRLITGRLQSLQTYVTQQLIRA |
| | AEIRASANLAATKMSECVLGQSKRVDFCGKGYHLMSFPQSAP |
| | HGVVFLHVTYVPAQEKNFTTAPAICHDGKAHFPREGVFVSNG |
| | THWFVTQRNFYEPQIITTDNTFVSGNCDVVIGIVNNTVYDPLQ |
| | PELDSFKEELDKYFKNHTSPDVDLGDISGINASVVNIQKEIDRL |
| | NEVAKNLNESLIDLQELGKYEQYIKWPWYIWLGFIAGLIAIVM |
| | VTIMLCCMTSCCSCLKGCCSCGSCCKFDEDDSEPVLKGVKLH |
| | YT |
| Optimized nucleotide | (SEQ ID NO: 34) |
| sequence encoding the | ATGCCTAATATCACTAACCTGTGTCCTTTCGGTGAAGTGTT |
| receptor-binding | CAACGCCACCAGGTTTGCTAGCGTGTATGCCTGGAACAGG |
| domain of a SARS-CoV- | AAGAGGATCTCTAACTGCGTCGCCGACTATTCCGTGCTGTA |
| 2 S protein | TAACAGCGCCTCCTTCTCCACATTCAAATGCTATGGAGTGA |
| | GCCCGACAAAACTGAACGATCTCTGCTTTACAAATGTCTAC |
| | GCCGACTCTTTTGTGATCAGAGGGGACGAGGTCCGGCAGA |
| | TCGCACCAGGACAGACAGGCAAGATTGCTGACTACAACTA |
| | TAAGCTGCCTGACGACTTCACAGGATGTGTGATCGCATGGA |
| | ACTCAAACAATCTGGACTCCAAAGTCGGGGGCAACTATAA |
| | TTACCTGTATCGCCTGTTCCGGAAGTCCAACCTGAAGCCCT |
| | TCGAGAGGGACATCAGTACAGAGATCTATCAGGCTGGCTC |
| | CACCCCTTGCAATGGCGTCGAAGGCTTTAATTGTTATTTTCC |
| | CCTGCAGTCTTACGGGTTTCAGCCTACTAATGGAGTTGGGT |
| | ACCAGCCATACAGAGTGGTCGTGCTCAGCTTCGAGCTCCTG |
| | CATGCTCCATAA |
| Receptor-binding | (SEQ ID NO: 6) |
| domain of a SARS-CoV- | MPNITNLCPFGEVFNATRFASVYAWNRKRISNCVADYSVLYN |
| 2 S protein | SASFSTFKCYGVSPTKLNDLCFTNVYADSFVIRGDEVRQIAPG |
| | QTGKIADYNYKLPDDFTGCVIAWNSNNLDSKVGGNYNYLYR |
| | LFRKSNLKPFERDISTEIYQAGSTPCNGVEGFNCYFPLQSYGFQ |
| | PTNGVGYQPYRVVVLSFELLHAP |
| Optimized nucleotide | (SEQ ID NO: 35) |
| sequence encoding the | ATGTTCGTCTTCCTCGTGCTGCTCCCACTCGTTTCTTCCCAG |
| receptor-binding | TGTCCTAATATCACTAACCTGTGTCCTTTCGGTGAAGTGTTC |
| domain of a SARS-CoV- | AACGCCACCAGGTTTGCTAGCGTGTATGCCTGGAACAGGA |
| 2 S protein with a signal | AGAGGATCTCTAACTGCGTCGCCGACTATTCCGTGCTGTAT |
| sequence | AACAGCGCCTCCTTCTCCACATTCAAATGCTATGGAGTGAG |
| | CCCGACAAAACTGAACGATCTCTGCTTTACAAATGTCTACG |
| | CCGACTCTTTTGTGATCAGAGGGGACGAGGTCCGGCAGATC |
| | GCACCAGGACAGACAGGCAAGATTGCTGACTACAACTATA |
| | AGCTGCCTGACGACTTCACAGGATGTGTGATCGCATGGAAC |
| | TCAAACAATCTGGACTCCAAAGTCGGGGGCAACTATAATT |
| | ACCTGTATCGCCTGTTCCGGAAGTCCAACCTGAAGCCCTTC |
| | GAGAGGGACATCAGTACAGAGATCTATCAGGCTGGCTCCA |
| | CCCCTTGCAATGGCGTCGAAGGCTTTAATTGTTATTTTCCCC |
| | TGCAGTCTTACGGGTTTCAGCCTACTAATGGAGTTGGGTAC |
| | CAGCCATACAGAGTGGTCGTGCTCAGCTTCGAGCTCCTGCA |
| | TGCTCCATAA |
| Receptor-binding | (SEQ ID NO: 8) |
| domain of a SARS-CoV- | MFVFLVLLPLVSSQCPNITNLCPFGEVFNATRFASVYAWNRKR |
| 2 S protein with a signal | ISNCVADYSVLYNSASFSTFKCYGVSPTKLNDLCFTNVYADSF |
| sequence | VIRGDEVRQIAPGQTGKIADYNYKLPDDFTGCVIAWNSNNLD |
| | SKVGGNYNYLYRLFRKSNLKPFERDISTEIYQAGSTPCNGVEG |
| | FNCYFPLQSYGFQPTNGVGYQPYRVVVLSFELLHAP |
| Optimized nucleotide | (SEQ ID NO: 42) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove a | CACCAACTCCTTCACCAGAGGCGTGTATTACCCAGACAAGG |
| furin cleavage site | TGTTTAGAAGCAGCGTGCTGCACTCTACCCAGGACCTCTTT |
| | CTGCCCTTTTTCAGCAACGTGACATGGTTTCACGCAATTCA |
| | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTGGCTCCGCCTCTTCCGTTGCTAGTCAGTCTATTATTGCC |
| | TATACCATGAGCCTCGGAGCTGAGAATAGCGTGGCCTACTC |
| | CAATAATTCCATCGCAATCCCTACTAACTTCACTATTTCTGT |
| | GACCACCGAGATCCTGCCTGTGTCTATGACTAAGACTAGCG |
| | TTGATTGTACCATGTATATTTGTGGCGACTCTACCGAATGTT |
| | CTAACCTGCTGCTTCAGTACGGCTCATTTTGCACACAGCTG |
| | AACAGAGCCCTGACTGGGATCGCTGTGGAGCAGGACAAGA |
| | ACACACAGGAGGTGTTTGCACAGGTGAAGCAGATCTATAA |
| | GACCCCTCCTATTAAGGATTTCGGCGGATTCAATTTCTCAC |
| | AGATTCTGCCAGACCCCAGTAAGCCTTCCAAGAGGAGCTTC |
| | ATCGAGGATCTCCTGTTTAACAAGGTGACCCTGGCAGACGC |
| | CGGCTTTATTAAGCAATATGGGGATTGCCTGGGCGACATTG |
| | CTGCCAGAGACCTGATTTGCGCCCAGAAATTCAATGGCCTC |
| | ACAGTGCTGCCACCTCTGCTGACCGACGAGATGATCGCTCA |
| | ATACACTAGCGCACTGCTGGCCGGAACCATCACATCAGGCT |
| | GGACCTTCGGGGCCGGAGCAGCACTGCAGATTCCATTCGCC |
| | ATGCAGATGGCCTATAGATTCAACGGCATTGGCGTCACACA |
| | GAACGTGCTGTACGAAAACCAGAAGCTCATCGCTAACCAG |
| | TTTAATTCCGCAATTGGAAAGATCCAAGATTCACTCAGCTC |
| | AACCGCCTCTGCACTCGGAAAGCTGCAGGACGTGGTCAAC |
| | CAGAATGCTCAGGCCCTGAACACACTCGTCAAGCAGCTGTC |
| | CTCTAACTTTGGCGCTATCAGCTCCGTTCTGAACGACATTCT |
| | GAGCCGCCTGGATAAGGTGGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTGACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGTATATCAA |
| | ATGGCCCTGGTACATTTGGCTGGGGTTTATCGCCGGACTGA |
| | TTGCCATCGTCATGGTGACCATCATGCTGTGTTGCATGACC |
| | TCCTGTTGTTCCTGTCTGAAGGGCTGCTGTAGTTGCGGCTCT |
| | TGCTGTAAATTCGACGAAGATGATAGCGAGCCCGTGCTGA |
| | AGGGCGTGAAGCTGCATTATACCTGA |
| SARS-CoV-2 S protein | (SEQ ID NO: 9) |
| mutated to remove a | MFVFLVLLPLVSSQCVNLTTRTQLPPAYTNSFTRGVYYPDKVF |
| furin cleavage site | RSSVLHSTQDLFLPFFSNVTWFHAIHVSGTNGTKRFDNPVLPF |
| | NDGVYFASTEKSNIIRGWIFGTTLDSKTQSLLIVNNATNVVIKV |
| | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDKVEAEV |
| | QIDRLITGRLQSLQTYVTQQLIRAAEIRASANLAATKMSECVL |
| | GQSKRVDFCGKGYHLMSFPQSAPHGVVFLHVTYVPAQEKNF |
| | TTAPAICHDGKAHFPREGVFVSNGTHWFVTQRNFYEPQIITTD |
| | NTFVSGNCDVVIGIVNNTVYDPLQPELDSFKEELDKYFKNHTS |
| | PDVDLGDISGINASVVNIQKEIDRLNEVAKNLNESLIDLQELGK |
| | YEQYIKWPWYIWLGFIAGLIAIVMVTIMLCCMTSCCSCLKGCC |
| | SCGSCCKFDEDDSEPVLKGVKLHYT |
| Optimized nucleotide | (SEQ ID NO: 43) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| with residues 986 and | CACCAACTCCTTCACCAGAGGCGTGTATTACCCAGACAAGG |
| 987 mutated to proline | TGTTTAGAAGCAGCGTGCTGCACTCTACCCAGGACCTCTTT |
| | CTGCCCTTTTTCAGCAACGTGACATGGTTTCACGCAATTCA |
| | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTAGAAGGGCCAGGTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCTT |
| | CATCGAGGATCTCCTGTTTAACAAGGTGACCCTGGCAGACG |
| | CCGGCTTTATTAAGCAATATGGGGATTGCCTGGGCGACATT |
| | GCTGCCAGAGACCTGATTTGCGCCCAGAAATTCAATGGCCT |
| | CACAGTGCTGCCACCTCTGCTGACCGACGAGATGATCGCTC |
| | AATACACTAGCGCACTGCTGGCCGGAACCATCACATCAGG |
| | CTGGACCTTCGGGGCCGGAGCAGCACTGCAGATTCCATTCG |
| | CCATGCAGATGGCCTATAGATTCAACGGCATTGGCGTCACA |
| | CAGAACGTGCTGTACGAAAACCAGAAGCTCATCGCTAACC |
| | AGTTTAATTCCGCAATTGGAAAGATCCAAGATTCACTCAGC |
| | TCAACCGCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGGATCCCCCAGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGTATATC |
| | AAATGGCCCTGGTACATTTGGCTGGGGTTTATCGCCGGACT |
| | GATTGCCATCGTCATGGTGACCATCATGCTGTGTTGCATGA |
| | CCTCCTGTTGTTCCTGTCTGAAGGGCTGCTGTAGTTGCGGCT |
| | CTTGCTGTAAATTCGACGAAGATGATAGCGAGCCCGTGCTG |
| | AAGGGCGTGAAGCTGCATTATACCTGA |
| SARS-CoV-2 S protein | (SEQ ID NO: 10) |
| with residues 986 and | MFVFLVLLPLVSSQCVNLTTRTQLPPAYTNSFTRGVYYPDKVF |
| 987 mutated to proline | RSSVLHSTQDLFLPFFSNVTWFHAIHVSGTNGTKRFDNPVLPF |
| | NDGVYFASTEKSNIIRGWIFGTTLDSKTQSLLIVNNATNVVIKV |
| | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPRRARSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 44) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove a | CACCAACTCCTTCACCAGAGGCGTGTATTACCCAGACAAGG |
| furin cleavage site and | TGTTTAGAAGCAGCGTGCTGCACTCTACCCAGGACCTCTTT |
| to replace residues 986 | CTGCCCTTTTTCAGCAACGTGACATGGTTTCACGCAATTCA |
| and 987 with proline | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTGGCTCCGCCTCTTCCGTTGCTAGTCAGTCTATTATTGCC |
| | TATACCATGAGCCTCGGAGCTGAGAATAGCGTGGCCTACTC |
| | CAATAATTCCATCGCAATCCCTACTAACTTCACTATTTCTGT |
| | GACCACCGAGATCCTGCCTGTGTCTATGACTAAGACTAGCG |
| | TTGATTGTACCATGTATATTTGTGGCGACTCTACCGAATGTT |
| | CTAACCTGCTGCTTCAGTACGGCTCATTTTGCACACAGCTG |
| | AACAGAGCCCTGACTGGGATCGCTGTGGAGCAGGACAAGA |
| | ACACACAGGAGGTGTTTGCACAGGTGAAGCAGATCTATAA |
| | GACCCCTCCTATTAAGGATTTCGGCGGATTCAATTTCTCAC |
| | AGATTCTGCCAGACCCCAGTAAGCCTTCCAAGAGGAGCTTC |
| | ATCGAGGATCTCCTGTTTAACAAGGTGACCCTGGCAGACGC |
| | CGGCTTTATTAAGCAATATGGGGATTGCCTGGGCGACATTG |
| | CTGCCAGAGACCTGATTTGCGCCCAGAAATTCAATGGCCTC |
| | ACAGTGCTGCCACCTCTGCTGACCGACGAGATGATCGCTCA |
| | ATACACTAGCGCACTGCTGGCCGGAACCATCACATCAGGCT |
| | GGACCTTCGGGGCCGGAGCAGCACTGCAGATTCCATTCGCC |
| | ATGCAGATGGCCTATAGATTCAACGGCATTGGCGTCACACA |
| | GAACGTGCTGTACGAAAACCAGAAGCTCATCGCTAACCAG |
| | TTTAATTCCGCAATTGGAAAGATCCAAGATTCACTCAGCTC |
| | AACCGCCTCTGCACTCGGAAAGCTGCAGGACGTGGTCAAC |
| | CAGAATGCTCAGGCCCTGAACACACTCGTCAAGCAGCTGTC |
| | CTCTAACTTTGGCGCTATCAGCTCCGTTCTGAACGACATTCT |
| | GAGCCGCCTGGATCCCCCAGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTGACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGTATATCAA |
| | ATGGCCCTGGTACATTTGGCTGGGGTTTATCGCCGGACTGA |
| | TTGCCATCGTCATGGTGACCATCATGCTGTGTTGCATGACC |
| | TCCTGTTGTTCCTGTCTGAAGGGCTGCTGTAGTTGCGGCTCT |
| | TGCTGTAAATTCGACGAAGATGATAGCGAGCCCGTGCTGA |
| | AGGGCGTGAAGCTGCATTATACCTGA |
| SARS-CoV-2 S protein | (SEQ ID NO: 11) |
| mutated to remove a | MFVFLVLLPLVSSQCVNLTTRTQLPPAYTNSFTRGVYYPDKVF |
| furin cleavage site and | RSSVLHSTQDLFLPFFSNVTWFHAIHVSGTNGTKRFDNPVLPF |
| to replace residues 986 | NDGVYFASTEKSNIIRGWIFGTTLDSKTQSLLIVNNATNVVIKV |
| and 987 with proline | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 45) |
| sequence encoding the | ATGTTCGTCTTCCTCGTGCTGCTCCCACTCGTTTCTTCCCAG |
| ectodomain of a SARS- | TGTGTCAACCTGACAACTAGGACTCAGCTGCCACCAGCCTA |
| CoV-2 S protein | CACCAACTCCTTCACCAGAGGCGTGTATTACCCAGACAAGG |
| mutated to remove a | TGTTTAGAAGCAGCGTGCTGCACTCTACCCAGGACCTCTTT |
| furin cleavage site and | CTGCCCTTTTTCAGCAACGTGACATGGTTTCACGCAATTCA |
| to replace residues 986 | CGTGTCCGGCACTAATGGCACAAAGCGGTTCGACAATCCA |
| and 987 with proline | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTGGCTCCGCCTCTTCCGTTGCTAGTCAGTCTATTATTGCC |
| | TATACCATGAGCCTCGGAGCTGAGAATAGCGTGGCCTACTC |
| | CAATAATTCCATCGCAATCCCTACTAACTTCACTATTTCTGT |
| | GACCACCGAGATCCTGCCTGTGTCTATGACTAAGACTAGCG |
| | TTGATTGTACCATGTATATTTGTGGCGACTCTACCGAATGTT |
| | CTAACCTGCTGCTTCAGTACGGCTCATTTTGCACACAGCTG |
| | AACAGAGCCCTGACTGGGATCGCTGTGGAGCAGGACAAGA |
| | ACACACAGGAGGTGTTTGCACAGGTGAAGCAGATCTATAA |
| | GACCCCTCCTATTAAGGATTTCGGCGGATTCAATTTCTCAC |
| | AGATTCTGCCAGACCCCAGTAAGCCTTCCAAGAGGAGCTTC |
| | ATCGAGGATCTCCTGTTTAACAAGGTGACCCTGGCAGACGC |
| | CGGCTTTATTAAGCAATATGGGGATTGCCTGGGCGACATTG |
| | CTGCCAGAGACCTGATTTGCGCCCAGAAATTCAATGGCCTC |
| | ACAGTGCTGCCACCTCTGCTGACCGACGAGATGATCGCTCA |
| | ATACACTAGCGCACTGCTGGCCGGAACCATCACATCAGGCT |
| | GGACCTTCGGGGCCGGAGCAGCACTGCAGATTCCATTCGCC |
| | ATGCAGATGGCCTATAGATTCAACGGCATTGGCGTCACACA |
| | GAACGTGCTGTACGAAAACCAGAAGCTCATCGCTAACCAG |
| | TTTAATTCCGCAATTGGAAAGATCCAAGATTCACTCAGCTC |
| | AACCGCCTCTGCACTCGGAAAGCTGCAGGACGTGGTCAAC |
| | CAGAATGCTCAGGCCCTGAACACACTCGTCAAGCAGCTGTC |
| | CTCTAACTTTGGCGCTATCAGCTCCGTTCTGAACGACATTCT |
| | GAGCCGCCTGGATCCCCCAGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTGACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGTGA |
| SARS-CoV-2 S protein | (SEQ ID NO: 118) |
| with residues 986 and | MFVFLVLLPLVSSQCVNLTTRTQLPPAYTNSFTRGVYYPDKVF |
| 987 mutated to proline, | RSSVLHSTQDLFLPFFSNVTWFHAIHVSGTNGTKRFDNPVLPF |
| and to contain the | NDGVYFASTEKSNIIRGWIFGTTLDSKTQSLLIVNNATNVVIKV |
| D614G mutation | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPRRARSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 119) |
| sequence encoding | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| with residues 986 and | CACCAACTCCTTCACCAGAGGCGTGTATTACCCAGACAAGG |
| 987 mutated to proline, | TGTTTAGAAGCAGCGTGCTGCACTCTACCCAGGACCTCTTT |
| and to contain the | CTGCCCTTTTTCAGCAACGTGACATGGTTTCACGCAATTCA |
| D614G mutation* | CGTGTCCGGCACTAATGGCACAAAGCGGTTCGACAATCCA |
| *underlined residues | GTCCTGCCTTTCAACGATGGCGTCTACTTTGCATCTACTGA |
| correspond to D614G | GAAATCCAATATCATTAGGGGATGGATCTTCGGCACAACCC |
| mutation location | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGGCGTCAATTGCACAGAAGTGCCAGTTGCTATCCACGC |
| | AGACCAGCTGACTCCCACATGGCGGGTGTATAGCACCGGA |
| | TCCAACGTGTTTCAGACCCGCGCCGGATGTCTCATTGGGGC |
| | CGAGCACGTGAATAACAGCTACGAGTGCGACATCCCCATT |
| | GGCGCCGGCATTTGTGCGTCTTACCAGACTCAGACCAACTC |
| | TCCTAGAAGGGCCAGGTCCGTTGCTAGTCAGTCTATTATTG |
| | CCTATACCATGAGCCTCGGAGCTGAGAATAGCGTGGCCTAC |
| | TCCAATAATTCCATCGCAATCCCTACTAACTTCACTATTTCT |
| | GTGACCACCGAGATCCTGCCTGTGTCTATGACTAAGACTAG |
| | CGTTGATTGTACCATGTATATTTGTGGCGACTCTACCGAAT |
| | GTTCTAACCTGCTGCTTCAGTACGGCTCATTTTGCACACAG |
| | CTGAACAGAGCCCTGACTGGGATCGCTGTGGAGCAGGACA |
| | AGAACACACAGGAGGTGTTTGCACAGGTGAAGCAGATCTA |
| | TAAGACCCCTCCTATTAAGGATTTCGGCGGATTCAATTTCT |
| | CACAGATTCTGCCAGACCCCAGTAAGCCTTCCAAGAGGAG |
| | CTTCATCGAGGATCTCCTGTTTAACAAGGTGACCCTGGCAG |
| | ACGCCGGCTTTATTAAGCAATATGGGGATTGCCTGGGCGAC |
| | ATTGCTGCCAGAGACCTGATTTGCGCCCAGAAATTCAATGG |
| | CCTCACAGTGCTGCCACCTCTGCTGACCGACGAGATGATCG |
| | CTCAATACACTAGCGCACTGCTGGCCGGAACCATCACATCA |
| | GGCTGGACCTTCGGGGCCGGAGCAGCACTGCAGATTCCATT |
| | CGCCATGCAGATGGCCTATAGATTCAACGGCATTGGCGTCA |
| | CACAGAACGTGCTGTACGAAAACCAGAAGCTCATCGCTAA |
| | CCAGTTTAATTCCGCAATTGGAAAGATCCAAGATTCACTCA |
| | GCTCAACCGCCTCTGCACTCGGAAAGCTGCAGGACGTGGTC |
| | AACCAGAATGCTCAGGCCCTGAACACACTCGTCAAGCAGC |
| | TGTCCTCTAACTTTGGCGCTATCAGCTCCGTTCTGAACGAC |
| | ATTCTGAGCCGCCTGGATCCCCCAGAGGCTGAAGTCCAGAT |
| | TGACCGCCTGATTACCGGCCGGCTGCAGTCTCTGCAAACAT |
| | ACGTGACCCAGCAGCTGATCAGAGCAGCCGAGATCCGGGC |
| | ATCCGCAAATCTGGCAGCAACTAAGATGAGCGAATGCGTG |
| | CTGGGCCAGTCCAAGCGGGTGGACTTTTGTGGCAAGGGCT |
| | ACCACCTGATGAGCTTCCCCCAGAGCGCCCCACATGGCGTT |
| | GTTTTTCTGCACGTGACCTATGTCCCTGCTCAGGAAAAGAA |
| | CTTTACAACTGCTCCTGCTATCTGCCATGACGGCAAGGCCC |
| | ACTTCCCACGGGAGGGAGTGTTTGTGTCCAATGGCACACAC |
| | TGGTTCGTGACCCAGAGGAACTTCTATGAACCCCAGATCAT |
| | CACCACTGACAATACCTTCGTGTCTGGAAATTGCGACGTCG |
| | TGATCGGCATCGTTAACAACACCGTGTACGACCCTCTCCAG |
| | CCAGAGCTGGACTCCTTTAAGGAGGAACTGGATAAGTATTT |
| | TAAGAACCACACAAGCCCAGATGTGGATCTCGGGGACATC |
| | TCCGGAATTAACGCCTCCGTGGTGAATATCCAGAAGGAGA |
| | TTGACCGCCTAAATGAAGTTGCCAAGAACCTCAATGAGTCT |
| | CTGATTGATCTGCAGGAACTGGGCAAGTATGAGCAGTATAT |
| | CAAATGGCCCTGGTACATTTGGCTGGGGTTTATCGCCGGAC |
| | TGATTGCCATCGTCATGGTGACCATCATGCTGTGTTGCATG |
| | ACCTCCTGTTGTTCCTGTCTGAAGGGCTGCTGTAGTTGCGG |
| | CTCTTGCTGTAAATTCGACGAAGATGATAGCGAGCCCGTGC |
| | TGAAGGGCGTGAAGCTGCATTATACCTGA |
| SARS-CoV-2 S protein | (SEQ ID NO: 120) |
| mutated to remove a | MFVFLVLLPLVSSQCVNLTTRTQLPPAYTNSFTRGVYYPDKVF |
| furin cleavage site and | RSSVLHSTQDLFLPFFSNVTWFHAIHVSGTNGTKRFDNPVLPF |
| to replace residues 986 | NDGVYFASTEKSNIIRGWIFGTTLDSKTQSLLIVNNATNVVIKV |
| and 987 with proline, | CEFQFCNDPFLGVYYHKNNKSWMESEFRVYSSANNCTFEYVS |
| and to contain the | QPFLMDLEGKQGNFKNLREFVFKNIDGYFKIYSKHTPINLVRD |
| D614G mutation | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQGVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 121) |
| sequence encoding | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove a | CACCAACTCCTTCACCAGAGGCGTGTATTACCCAGACAAGG |
| furin cleavage site and | TGTTTAGAAGCAGCGTGCTGCACTCTACCCAGGACCTCTTT |
| to replace residues 986 | CTGCCCTTTTTCAGCAACGTGACATGGTTTCACGCAATTCA |
| and 987 with proline, | CGTGTCCGGCACTAATGGCACAAAGCGGTTCGACAATCCA |
| and to contain the | GTCCTGCCTTTCAACGATGGCGTCTACTTTGCATCTACTGA |
| D614G mutation* | GAAATCCAATATCATTAGGGGATGGATCTTCGGCACAACCC |
| *underlined residues | TGGATTCTAAGACCCAGAGCCTGCTGATCGTCAACAACGCC |
| correspond to D614G | ACAAACGTGGTCATTAAGGTTTGCGAGTTTCAGTTCTGTAA |
| mutation location | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGGCGTCAATTGCACAGAAGTGCCAGTTGCTATCCACGC |
| | AGACCAGCTGACTCCCACATGGCGGGTGTATAGCACCGGA |
| | TCCAACGTGTTTCAGACCCGCGCCGGATGTCTCATTGGGGC |
| | CGAGCACGTGAATAACAGCTACGAGTGCGACATCCCCATT |
| | GGCGCCGGCATTTGTGCGTCTTACCAGACTCAGACCAACTC |
| | TCCTGGCTCCGCCTCTTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCTT |
| | CATCGAGGATCTCCTGTTTAACAAGGTGACCCTGGCAGACG |
| | CCGGCTTTATTAAGCAATATGGGGATTGCCTGGGCGACATT |
| | GCTGCCAGAGACCTGATTTGCGCCCAGAAATTCAATGGCCT |
| | CACAGTGCTGCCACCTCTGCTGACCGACGAGATGATCGCTC |
| | AATACACTAGCGCACTGCTGGCCGGAACCATCACATCAGG |
| | CTGGACCTTCGGGGCCGGAGCAGCACTGCAGATTCCATTCG |
| | CCATGCAGATGGCCTATAGATTCAACGGCATTGGCGTCACA |
| | CAGAACGTGCTGTACGAAAACCAGAAGCTCATCGCTAACC |
| | AGTTTAATTCCGCAATTGGAAAGATCCAAGATTCACTCAGC |
| | TCAACCGCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGGATCCCCCAGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGTATATC |
| | AAATGGCCCTGGTACATTTGGCTGGGGTTTATCGCCGGACT |
| | GATTGCCATCGTCATGGTGACCATCATGCTGTGTTGCATGA |
| | CCTCCTGTTGTTCCTGTCTGAAGGGCTGCTGTAGTTGCGGCT |
| | CTTGCTGTAAATTCGACGAAGATGATAGCGAGCCCGTGCTG |
| | AAGGGCGTGAAGCTGCATTATACCTGA |
| Ectodomain of a SARS- | (SEQ ID NO: 12) |
| CoV-2 S protein | MFVFLVLLPLVSSQCVNLTTRTQLPPAYTNSFTRGVYYPDKVF |
| mutated to remove a | RSSVLHSTQDLFLPFFSNVTWFHAIHVSGTNGTKRFDNPVLPF |
| furin cleavage site and | NDGVYFASTEKSNIIRGWIFGTTLDSKTQSLLIVNNATNVVIKV |
| to replace residues 986 | CEFQFCNDPFLGVYYHKNNKSWMESEFRVYSSANNCTFEYVS |
| and 987 with proline | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | Q |
| Optimized nucleotide | (SEQ ID NO: 46) |
| sequence encoding the | ATGTTCGTCTTCCTCGTGCTGCTCCCACTCGTTTCTTCCCAG |
| ectodomain of the | TGTGTCAACCTGACAACTAGGACTCAGCTGCCACCAGCCTA |
| SARS-CoV-2 S protein | CACCAACTCCTTCACCAGAGGCGTGTATTACCCAGACAAGG |
| with a Foldon | TGTTTAGAAGCAGCGTGCTGCACTCTACCCAGGACCTCTTT |
| | CTGCCCTTTTTCAGCAACGTGACATGGTTTCACGCAATTCA |
| | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTAGAAGGGCCAGGTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCTT |
| | CATCGAGGATCTCCTGTTTAACAAGGTGACCCTGGCAGACG |
| | CCGGCTTTATTAAGCAATATGGGGATTGCCTGGGCGACATT |
| | GCTGCCAGAGACCTGATTTGCGCCCAGAAATTCAATGGCCT |
| | CACAGTGCTGCCACCTCTGCTGACCGACGAGATGATCGCTC |
| | AATACACTAGCGCACTGCTGGCCGGAACCATCACATCAGG |
| | CTGGACCTTCGGGGCCGGAGCAGCACTGCAGATTCCATTCG |
| | CCATGCAGATGGCCTATAGATTCAACGGCATTGGCGTCACA |
| | CAGAACGTGCTGTACGAAAACCAGAAGCTCATCGCTAACC |
| | AGTTTAATTCCGCAATTGGAAAGATCCAAGATTCACTCAGC |
| | TCAACCGCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGGATAAGGTGGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGGGGTA |
| | CATTCCCGAGGCTCCTAGGGACGGCCAGGCATACGTGCGC |
| | AAAGACGGCGAGTGGGTGCTGCTGTCCACATTCCTGTAA |
| Ectodomain of a SARS- | (SEQ ID NO: 14) |
| CoV-2 S protein with a | MFVFLVLLPLVSSQCVNLTTRTQLPPAYTNSFTRGVYYPDKVF |
| Foldon | RSSVLHSTQDLFLPFFSNVTWFHAIHVSGTNGTKRFDNPVLPF |
| | NDGVYFASTEKSNIIRGWIFGTTLDSKTQSLLIVNNATNVVIKV |
| | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPRRARSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDKVEAEV |
| | QIDRLITGRLQSLQTYVTQQLIRAAEIRASANLAATKMSECVL |
| | GQSKRVDFCGKGYHLMSFPQSAPHGVVFLHVTYVPAQEKNF |
| | TTAPAICHDGKAHFPREGVFVSNGTHWFVTQRNFYEPQIITTD |
| | NTFVSGNCDVVIGIVNNTVYDPLQPELDSFKEELDKYFKNHTS |
| | PDVDLGDISGINASVVNIQKEIDRLNEVAKNLNESLIDLQELGK |
| | YEQGYIPEAPRDGQAYVRKDGEWVLLSTFL |
| Optimized nucleotide | (SEQ ID NO: 47) |
| sequence encoding the | ATGTTCGTCTTCCTCGTGCTGCTCCCACTCGTTTCTTCCCAG |
| S2′ subunit of a SARS- | TGTAGCTTCATCGAGGATCTCCTGTTTAACAAGGTGACCCT |
| CoV-2 S protein with a | GGCAGACGCCGGCTTTATTAAGCAATATGGGGATTGCCTGG |
| Foldon and a signal | GCGACATTGCTGCCAGAGACCTGATTTGCGCCCAGAAATTC |
| sequence | AATGGCCTCACAGTGCTGCCACCTCTGCTGACCGACGAGAT |
| | GATCGCTCAATACACTAGCGCACTGCTGGCCGGAACCATCA |
| | CATCAGGCTGGACCTTCGGGGCCGGAGCAGCACTGCAGAT |
| | TCCATTCGCCATGCAGATGGCCTATAGATTCAACGGCATTG |
| | GCGTCACACAGAACGTGCTGTACGAAAACCAGAAGCTCAT |
| | CGCTAACCAGTTTAATTCCGCAATTGGAAAGATCCAAGATT |
| | CACTCAGCTCAACCGCCTCTGCACTCGGAAAGCTGCAGGAC |
| | GTGGTCAACCAGAATGCTCAGGCCCTGAACACACTCGTCA |
| | AGCAGCTGTCCTCTAACTTTGGCGCTATCAGCTCCGTTCTG |
| | AACGACATTCTGAGCCGCCTGGATAAGGTGGAGGCTGAAG |
| | TCCAGATTGACCGCCTGATTACCGGCCGGCTGCAGTCTCTG |
| | CAAACATACGTGACCCAGCAGCTGATCAGAGCAGCCGAGA |
| | TCCGGGCATCCGCAAATCTGGCAGCAACTAAGATGAGCGA |
| | ATGCGTGCTGGGCCAGTCCAAGCGGGTGGACTTTTGTGGCA |
| | AGGGCTACCACCTGATGAGCTTCCCCCAGAGCGCCCCACAT |
| | GGCGTTGTTTTTCTGCACGTGACCTATGTCCCTGCTCAGGA |
| | AAAGAACTTTACAACTGCTCCTGCTATCTGCCATGACGGCA |
| | AGGCCCACTTCCCACGGGAGGGAGTGTTTGTGTCCAATGGC |
| | ACACACTGGTTCGTGACCCAGAGGAACTTCTATGAACCCCA |
| | GATCATCACCACTGACAATACCTTCGTGTCTGGAAATTGCG |
| | ACGTCGTGATCGGCATCGTTAACAACACCGTGTACGACCCT |
| | CTCCAGCCAGAGCTGGACTCCTTTAAGGAGGAACTGGATA |
| | AGTATTTTAAGAACCACACAAGCCCAGATGTGGATCTCGG |
| | GGACATCTCCGGAATTAACGCCTCCGTGGTGAATATCCAGA |
| | AGGAGATTGACCGCCTAAATGAAGTTGCCAAGAACCTCAA |
| | TGAGTCTCTGATTGATCTGCAGGAACTGGGCAAGTATGAGC |
| | AGGGGTACATTCCCGAGGCTCCTAGGGACGGCCAGGCATA |
| | CGTGCGCAAAGACGGCGAGTGGGTGCTGCTGTCCACATTCC |
| | TGTAA |
| S2′ subunit of a SARS- | (SEQ ID NO: 15) |
| CoV-2 S protein with a | MFVFLVLLPLVSSQCSFIEDLLFNKVTLADAGFIKQYGDCLGDI |
| Foldon and a signal | AARDLICAQKFNGLTVLPPLLTDEMIAQYTSALLAGTITSGWT |
| sequence | FGAGAALQIPFAMQMAYRFNGIGVTQNVLYENQKLIANQFNS |
| | AIGKIQDSLSSTASALGKLQDVVNQNAQALNTLVKQLSSNFG |
| | AISSVLNDILSRLDKVEAEVQIDRLITGRLQSLQTYVTQQLIRA |
| | AEIRASANLAATKMSECVLGQSKRVDFCGKGYHLMSFPQSAP |
| | HGVVFLHVTYVPAQEKNFTTAPAICHDGKAHFPREGVFVSNG |
| | THWFVTQRNFYEPQIITTDNTFVSGNCDVVIGIVNNTVYDPLQ |
| | PELDSFKEELDKYFKNHTSPDVDLGDISGINASVVNIQKEIDRL |
| | NEVAKNLNESLIDLQELGKYEQGYIPEAPRDGQAYVRKDGEWV |
| | LLSTFL |
| Optimized nucleotide | (SEQ ID NO: 48) |
| sequence encoding the | ATGTTCGTCTTCCTCGTGCTGCTCCCACTCGTTTCTTCCCAG |
| ectodomain of a SARS- | TGTGTCAACCTGACAACTAGGACTCAGCTGCCACCAGCCTA |
| CoV-2 S protein, which | CACCAACTCCTTCACCAGAGGCGTGTATTACCCAGACAAGG |
| has been modified by | TGTTTAGAAGCAGCGTGCTGCACTCTACCCAGGACCTCTTT |
| mutating residues 986 | CTGCCCTTTTTCAGCAACGTGACATGGTTTCACGCAATTCA |
| and 987 to proline, with | CGTGTCCGGCACTAATGGCACAAAGCGGTTCGACAATCCA |
| a Foldon | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTAGAAGGGCCAGGTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCTT |
| | CATCGAGGATCTCCTGTTTAACAAGGTGACCCTGGCAGACG |
| | CCGGCTTTATTAAGCAATATGGGGATTGCCTGGGCGACATT |
| | GCTGCCAGAGACCTGATTTGCGCCCAGAAATTCAATGGCCT |
| | CACAGTGCTGCCACCTCTGCTGACCGACGAGATGATCGCTC |
| | AATACACTAGCGCACTGCTGGCCGGAACCATCACATCAGG |
| | CTGGACCTTCGGGGCCGGAGCAGCACTGCAGATTCCATTCG |
| | CCATGCAGATGGCCTATAGATTCAACGGCATTGGCGTCACA |
| | CAGAACGTGCTGTACGAAAACCAGAAGCTCATCGCTAACC |
| | AGTTTAATTCCGCAATTGGAAAGATCCAAGATTCACTCAGC |
| | TCAACCGCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGGATCCCCCAGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGGGGTA |
| | CATTCCCGAGGCTCCTAGGGACGGCCAGGCATACGTGCGC |
| | AAAGACGGCGAGTGGGTGCTGCTGTCCACATTCCTGTAA |
| Ectodomain of a SARS- | (SEQ ID NO: 16) |
| CoV-2 S protein, which | MFVFLVLLPLVSSQCVNLTTRTQLPPAYTNSFTRGVYYPDKVF |
| has been modified by | RSSVLHSTQDLFLPFFSNVTWFHAIHVSGTNGTKRFDNPVLPF |
| mutating residues 986 | NDGVYFASTEKSNIIRGWIFGTTLDSKTQSLLIVNNATNVVIKV |
| and 987 to proline, with | CEFQFCNDPFLGVYYHKNNKSWMESEFRVYSSANNCTFEYVS |
| a Foldon | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGENCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPRRARSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QGYIPEAPRDGQAYVRKDGEWVLLSTFL |
| Optimized nucleotide | (SEQ ID NO: 49) |
| sequence encoding the | ATGTTCGTCTTCCTCGTGCTGCTCCCACTCGTTTCTTCCCAG |
| ectodomain of a SARS- | TGTGTCAACCTGACAACTAGGACTCAGCTGCCACCAGCCTA |
| CoV-2 S protein, which | CACCAACTCCTTCACCAGAGGCGTGTATTACCCAGACAAGG |
| has been modified to | TGTTTAGAAGCAGCGTGCTGCACTCTACCCAGGACCTCTTT |
| remove the furin | CTGCCCTTTTTCAGCAACGTGACATGGTTTCACGCAATTCA |
| cleavage site and to | CGTGTCCGGCACTAATGGCACAAAGCGGTTCGACAATCCA |
| replace residues 986 and | GTCCTGCCTTTCAACGATGGCGTCTACTTTGCATCTACTGA |
| 987 with proline, with a | GAAATCCAATATCATTAGGGGATGGATCTTCGGCACAACCC |
| C terminal Foldon | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTGGCTCCGCCTCTTCCGTTGCTAGTCAGTCTATTATTGCC |
| | TATACCATGAGCCTCGGAGCTGAGAATAGCGTGGCCTACTC |
| | CAATAATTCCATCGCAATCCCTACTAACTTCACTATTTCTGT |
| | GACCACCGAGATCCTGCCTGTGTCTATGACTAAGACTAGCG |
| | TTGATTGTACCATGTATATTTGTGGCGACTCTACCGAATGTT |
| | CTAACCTGCTGCTTCAGTACGGCTCATTTTGCACACAGCTG |
| | AACAGAGCCCTGACTGGGATCGCTGTGGAGCAGGACAAGA |
| | ACACACAGGAGGTGTTTGCACAGGTGAAGCAGATCTATAA |
| | GACCCCTCCTATTAAGGATTTCGGCGGATTCAATTTCTCAC |
| | AGATTCTGCCAGACCCCAGTAAGCCTTCCAAGAGGAGCTTC |
| | ATCGAGGATCTCCTGTTTAACAAGGTGACCCTGGCAGACGC |
| | CGGCTTTATTAAGCAATATGGGGATTGCCTGGGCGACATTG |
| | CTGCCAGAGACCTGATTTGCGCCCAGAAATTCAATGGCCTC |
| | ACAGTGCTGCCACCTCTGCTGACCGACGAGATGATCGCTCA |
| | ATACACTAGCGCACTGCTGGCCGGAACCATCACATCAGGCT |
| | GGACCTTCGGGGCCGGAGCAGCACTGCAGATTCCATTCGCC |
| | ATGCAGATGGCCTATAGATTCAACGGCATTGGCGTCACACA |
| | GAACGTGCTGTACGAAAACCAGAAGCTCATCGCTAACCAG |
| | TTTAATTCCGCAATTGGAAAGATCCAAGATTCACTCAGCTC |
| | AACCGCCTCTGCACTCGGAAAGCTGCAGGACGTGGTCAAC |
| | CAGAATGCTCAGGCCCTGAACACACTCGTCAAGCAGCTGTC |
| | CTCTAACTTTGGCGCTATCAGCTCCGTTCTGAACGACATTCT |
| | GAGCCGCCTGGATCCCCCAGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTGACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGGGGTACAT |
| | TCCCGAGGCTCCTAGGGACGGCCAGGCATACGTGCGCAAA |
| | GACGGCGAGTGGGTGCTGCTGTCCACATTCCTGTAA |
| Ectodomain of a SARS- | (SEQ ID NO: 17) |
| CoV-2 S protein, which | MFVFLVLLPLVSSQCVNLTTRTQLPPAYTNSFTRGVYYPDKVF |
| has been modified to | RSSVLHSTQDLFLPFFSNVTWFHAIHVSGTNGTKRFDNPVLPF |
| remove a furin cleavage | NDGVYFASTEKSNIIRGWIFGTTLDSKTQSLLIVNNATNVVIKV |
| site and to replace | CEFQFCNDPFLGVYYHKNNKSWMESEFRVYSSANNCTFEYVS |
| residues 986 and 987 | QPFLMDLEGKQGNFKNLREFVFKNIDGYFKIYSKHTPINLVRD |
| with proline, with a C | LPQGFSALEPLVDLPIGINITRFQTLLALHRSYLTPGDSSSGWT |
| terminal Foldon | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QGYIPEAPRDGQAYVRKDGEWVLLSTFL |
| Optimized nucleotide | (SEQ ID NO: 50) |
| sequence encoding the | ATGCCTAATATCACTAACCTGTGTCCTTTCGGTGAAGTGTT |
| receptor-binding | CAACGCCACCAGGTTTGCTAGCGTGTATGCCTGGAACAGG |
| domain of a SARS-CoV- | AAGAGGATCTCTAACTGCGTCGCCGACTATTCCGTGCTGTA |
| 2 S protein with an Fc | TAACAGCGCCTCCTTCTCCACATTCAAATGCTATGGAGTGA |
| region | GCCCGACAAAACTGAACGATCTCTGCTTTACAAATGTCTAC |
| | GCCGACTCTTTTGTGATCAGAGGGGACGAGGTCCGGCAGA |
| | TCGCACCAGGACAGACAGGCAAGATTGCTGACTACAACTA |
| | TAAGCTGCCTGACGACTTCACAGGATGTGTGATCGCATGGA |
| | ACTCAAACAATCTGGACTCCAAAGTCGGGGGCAACTATAA |
| | TTACCTGTATCGCCTGTTCCGGAAGTCCAACCTGAAGCCCT |
| | TCGAGAGGGACATCAGTACAGAGATCTATCAGGCTGGCTC |
| | CACCCCTTGCAATGGCGTCGAAGGCTTTAATTGTTATTTTCC |
| | CCTGCAGTCTTACGGGTTTCAGCCTACTAATGGAGTTGGGT |
| | ACCAGCCATACAGAGTGGTCGTGCTCAGCTTCGAGCTCCTG |
| | CATGCTCCACCTAAGTCCTGCGACAAAACCCATACATGTCC |
| | ACCATGCCCAGCTCCTGAACTGCTCGGCGGGCCTAGTGTTT |
| | TCCTCTTCCCTCCTAAGCCCAAGGATACCCTCATGATCTCTC |
| | GCACACCAGAAGTGACCTGCGTGGTCGTGGATGTCTCTCAC |
| | GAGGATCCTGAAGTGAAGTTTAACTGGTATGTCGACGGAG |
| | TGGAAGTGCACAACGCCAAGACAAAGCCAAGAGAAGAAC |
| | AATACAATTCTACTTATAGGGTGGTGTCTGTGCTGACAGTG |
| | CTGCACCAGGATTGGCTGAATGGAAAAGAATATAAGTGTA |
| | AGGTCTCTAACAAGGCCCTGCCCGCTCCAATTGAGAAGAC |
| | AATTTCCAAGGCCAAGGGGCAGCCTCGGGAACCTCAGGTG |
| | TACACACTGCCCCCATCCAGGGATGAACTGACTAAAAATC |
| | AGGTGTCTCTGACATGCCTGGTGAAAGGGTTTTATCCAAGT |
| | GACATTGCTGTGGAGTGGGAGTCTAATGGGCAGCCTGAAA |
| | ATAACTACAAGACCACACCACCAGTGCTCGATAGCGACGG |
| | GTCTTTCTTTCTGTATTCTAAACTGACCGTGGATAAATCTCG |
| | GTGGCAGCAGGGAAACGTGTTTTCTTGCTCAGTGATGCACG |
| | AAGCTCTGCACAATCACTATACACAGAAATCCCTGTCCCTG |
| | TCTCCAGGCAAATAA |
| Receptor-binding | (SEQ ID NO: 19) |
| domain of a SARS-CoV- | MNITNLCPFGEVFNATRFASVYAWNRKRISNCVADYSVLYNS |
| 2 S protein with an Fc | ASFSTFKCYGVSPTKLNDLCFTNVYADSFVIRGDEVRQIAPGQ |
| region | TGKIADYNYKLPDDFTGCVIAWNSNNLDSKVGGNYNYLYRL |
| | FRKSNLKPFERDISTEIYQAGSTPCNGVEGFNCYFPLQSYGFQP |
| | TNGVGYQPYRVVVLSFELLHAPPKSCDKTHTCPPCPAPELLGG |
| | PSVFLFPPKPKDTLMISRTPEVTCVVVDVSHEDPEVKFNWYVDGV |
| | EVHNAKTKPREEQYNSTYRVVSVLTVLHQDWLNGKEYKCKVSNK |
| | ALPAPIEKTISKAKGQPREPQVYTLPPSRDELTKNQVSLTCLVKGF |
| | YPSDIAVEWESNGQPENNYKTTPPVLDSDGSFFLYSKLTVDKSRW |
| | QQGNVFSCSVMHEALHNHYTQKSLSLSPGK |
| Optimized nucleotide | (SEQ ID NO: 36) |
| sequence encoding a | ATGTTCGTCTTCCTCGTGCTGCTCCCACTCGTTTCTTCCCAG |
| receptor-binding | TGTCCTAATATCACTAACCTGTGTCCTTTCGGTGAAGTGTTC |
| domain of SARS-CoV-2 | AACGCCACCAGGTTTGCTAGCGTGTATGCCTGGAACAGGA |
| S protein with a signal | AGAGGATCTCTAACTGCGTCGCCGACTATTCCGTGCTGTAT |
| sequence and an Fc | AACAGCGCCTCCTTCTCCACATTCAAATGCTATGGAGTGAG |
| region | CCCGACAAAACTGAACGATCTCTGCTTTACAAATGTCTACG |
| | CCGACTCTTTTGTGATCAGAGGGGACGAGGTCCGGCAGATC |
| | GCACCAGGACAGACAGGCAAGATTGCTGACTACAACTATA |
| | AGCTGCCTGACGACTTCACAGGATGTGTGATCGCATGGAAC |
| | TCAAACAATCTGGACTCCAAAGTCGGGGGCAACTATAATT |
| | ACCTGTATCGCCTGTTCCGGAAGTCCAACCTGAAGCCCTTC |
| | GAGAGGGACATCAGTACAGAGATCTATCAGGCTGGCTCCA |
| | CCCCTTGCAATGGCGTCGAAGGCTTTAATTGTTATTTTCCCC |
| | TGCAGTCTTACGGGTTTCAGCCTACTAATGGAGTTGGGTAC |
| | CAGCCATACAGAGTGGTCGTGCTCAGCTTCGAGCTCCTGCA |
| | TGCTCCACCTAAGTCCTGCGACAAAACCCATACATGTCCAC |
| | CATGCCCAGCTCCTGAACTGCTCGGCGGGCCTAGTGTTTTC |
| | CTCTTCCCTCCTAAGCCCAAGGATACCCTCATGATCTCTCG |
| | CACACCAGAAGTGACCTGCGTGGTCGTGGATGTCTCTCACG |
| | AGGATCCTGAAGTGAAGTTTAACTGGTATGTCGACGGAGT |
| | GGAAGTGCACAACGCCAAGACAAAGCCAAGAGAAGAACA |
| | ATACAATTCTACTTATAGGGTGGTGTCTGTGCTGACAGTGC |
| | TGCACCAGGATTGGCTGAATGGAAAAGAATATAAGTGTAA |
| | GGTCTCTAACAAGGCCCTGCCCGCTCCAATTGAGAAGACA |
| | ATTTCCAAGGCCAAGGGGCAGCCTCGGGAACCTCAGGTGT |
| | ACACACTGCCCCCATCCAGGGATGAACTGACTAAAAATCA |
| | GGTGTCTCTGACATGCCTGGTGAAAGGGTTTTATCCAAGTG |
| | ACATTGCTGTGGAGTGGGAGTCTAATGGGCAGCCTGAAAA |
| | TAACTACAAGACCACACCACCAGTGCTCGATAGCGACGGG |
| | TCTTTCTTTCTGTATTCTAAACTGACCGTGGATAAATCTCGG |
| | TGGCAGCAGGGAAACGTGTTTTCTTGCTCAGTGATGCACGA |
| | AGCTCTGCACAATCACTATACACAGAAATCCCTGTCCCTGT |
| | CTCCAGGCAAATAA |
| Receptor-binding | (SEQ ID NO: 20) |
| domain of a SARS-CoV- | MFVFLVLLPLVSSQCPNITNLCPFGEVFNATRFASVYAWNRKR |
| 2 S protein with a signal | ISNCVADYSVLYNSASFSTFKCYGVSPTKLNDLCFTNVYADSF |
| sequence and an Fc | VIRGDEVRQIAPGQTGKIADYNYKLPDDFTGCVIAWNSNNLD |
| region | SKVGGNYNYLYRLFRKSNLKPFERDISTEIYQAGSTPCNGVEG |
| | FNCYFPLQSYGFQPTNGVGYQPYRVVVLSFELLHAPPKSCDKT |
| | HTCPPCPAPELLGGPSVFLFPPKPKDTLMISRTPEVTCVVVDVSH |
| | EDPEVKFNWYVDGVEVHNAKTKPREEQYNSTYRVVSVLTVLHQD |
| | WLNGKEYKCKVSNKALPAPIEKTISKAKGQPREPQVYTLPPSRDE |
| | LTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| Optimized nucleotide | (SEQ ID NO: 69) |
| sequence encoding a S2 | ATGTTCGTCTTCCTCGTGCTGCTCCCACTCGTTTCTTCCCAG |
| subunit of a SARS-CoV- | TGTTCCGTTGCTAGTCAGTCTATTATTGCCTATACCATGAGC |
| 2 S protein, which has | CTCGGAGCTGAGAATAGCGTGGCCTACTCCAATAATTCCAT |
| been modified to remove | CGCAATCCCTACTAACTTCACTATTTCTGTGACCACCGAGA |
| residues 986 and 987 | TCCTGCCTGTGTCTATGACTAAGACTAGCGTTGATTGTACC |
| with proline, with a | ATGTATATTTGTGGCGACTCTACCGAATGTTCTAACCTGCT |
| signal sequence | GCTTCAGTACGGCTCATTTTGCACACAGCTGAACAGAGCCC |
| | TGACTGGGATCGCTGTGGAGCAGGACAAGAACACACAGGA |
| | GGTGTTTGCACAGGTGAAGCAGATCTATAAGACCCCTCCTA |
| | TTAAGGATTTCGGCGGATTCAATTTCTCACAGATTCTGCCA |
| | GACCCCAGTAAGCCTTCCAAGAGGAGCTTCATCGAGGATCT |
| | CCTGTTTAACAAGGTGACCCTGGCAGACGCCGGCTTTATTA |
| | AGCAATATGGGGATTGCCTGGGCGACATTGCTGCCAGAGA |
| | CCTGATTTGCGCCCAGAAATTCAATGGCCTCACAGTGCTGC |
| | CACCTCTGCTGACCGACGAGATGATCGCTCAATACACTAGC |
| | GCACTGCTGGCCGGAACCATCACATCAGGCTGGACCTTCGG |
| | GGCCGGAGCAGCACTGCAGATTCCATTCGCCATGCAGATG |
| | GCCTATAGATTCAACGGCATTGGCGTCACACAGAACGTGCT |
| | GTACGAAAACCAGAAGCTCATCGCTAACCAGTTTAATTCCG |
| | CAATTGGAAAGATCCAAGATTCACTCAGCTCAACCGCCTCT |
| | GCACTCGGAAAGCTGCAGGACGTGGTCAACCAGAATGCTC |
| | AGGCCCTGAACACACTCGTCAAGCAGCTGTCCTCTAACTTT |
| | GGCGCTATCAGCTCCGTTCTGAACGACATTCTGAGCCGCCT |
| | GGATCCCCCAGAGGCTGAAGTCCAGATTGACCGCCTGATTA |
| | CCGGCCGGCTGCAGTCTCTGCAAACATACGTGACCCAGCA |
| | GCTGATCAGAGCAGCCGAGATCCGGGCATCCGCAAATCTG |
| | GCAGCAACTAAGATGAGCGAATGCGTGCTGGGCCAGTCCA |
| | AGCGGGTGGACTTTTGTGGCAAGGGCTACCACCTGATGAG |
| | CTTCCCCCAGAGCGCCCCACATGGCGTTGTTTTTCTGCACG |
| | TGACCTATGTCCCTGCTCAGGAAAAGAACTTTACAACTGCT |
| | CCTGCTATCTGCCATGACGGCAAGGCCCACTTCCCACGGGA |
| | GGGAGTGTTTGTGTCCAATGGCACACACTGGTTCGTGACCC |
| | AGAGGAACTTCTATGAACCCCAGATCATCACCACTGACAAT |
| | ACCTTCGTGTCTGGAAATTGCGACGTCGTGATCGGCATCGT |
| | TAACAACACCGTGTACGACCCTCTCCAGCCAGAGCTGGACT |
| | CCTTTAAGGAGGAACTGGATAAGTATTTTAAGAACCACAC |
| | AAGCCCAGATGTGGATCTCGGGGACATCTCCGGAATTAAC |
| | GCCTCCGTGGTGAATATCCAGAAGGAGATTGACCGCCTAA |
| | ATGAAGTTGCCAAGAACCTCAATGAGTCTCTGATTGATCTG |
| | CAGGAACTGGGCAAGTATGAGCAGTATATCAAATGGCCCT |
| | GGTACATTTGGCTGGGGTTTATCGCCGGACTGATTGCCATC |
| | GTCATGGTGACCATCATGCTGTGTTGCATGACCTCCTGTTG |
| | TTCCTGTCTGAAGGGCTGCTGTAGTTGCGGCTCTTGCTGTA |
| | AATTCGACGAAGATGATAGCGAGCCCGTGCTGAAGGGCGT |
| | GAAGCTGCATTATACCTGA |
| S2 subunit of a SARS- | (SEQ ID NO: 70) |
| CoV-2 S protein, which | MFVFLVLLPLVSSQCSVASQSIIAYTMSLGAENSVAYSNNSIAI |
| has been modified to | PTNFTISVTTEILPVSMTKTSVDCTMYICGDSTECSNLLLQYGS |
| remove residues 986 and | FCTQLNRALTGIAVEQDKNTQEVFAQVKQIYKTPPIKDFGGFN |
| 987 with proline, with a | FSQILPDPSKPSKRSFIEDLLFNKVTLADAGFIKQYGDCLGDIA |
| signal sequence | ARDLICAQKFNGLTVLPPLLTDEMIAQYTSALLAGTITSGWTF |
| | GAGAALQIPFAMQMAYRFNGIGVTQNVLYENQKLIANQFNSA |
| | IGKIQDSLSSTASALGKLQDVVNQNAQALNTLVKQLSSNFGAI |
| | SSVLNDILSRLDPPEAEVQIDRLITGRLQSLQTYVTQQLIRAAEI |
| | RASANLAATKMSECVLGQSKRVDFCGKGYHLMSFPQSAPHG |
| | VVFLHVTYVPAQEKNFTTAPAICHDGKAHFPREGVFVSNGTH |
| | WFVTQRNFYEPQIITTDNTFVSGNCDVVIGIVNNTVYDPLQPE |
| | LDSFKEELDKYFKNHTSPDVDLGDISGINASVVNIQKEIDRLNE |
| | VAKNLNESLIDLQELGKYEQYIKWPWYIWLGFIAGLIAIVMVT |
| | IMLCCMTSCCSCLKGCCSCGSCCKFDEDDSEPVLKGVKLHYT |
| Optimized nucleotide | (SEQ ID NO: 75) |
| sequence encoding a | ATGTTCGTCTTCCTCGTGCTGCTCCCACTCGTTTCTTCCCAG |
| ectodomain of the S2 | TGTTCCGTTGCTAGTCAGTCTATTATTGCCTATACCATGAGC |
| subunit of a SARS-CoV- | CTCGGAGCTGAGAATAGCGTGGCCTACTCCAATAATTCCAT |
| 2 S protein with a signal | CGCAATCCCTACTAACTTCACTATTTCTGTGACCACCGAGA |
| sequence and a Foldon | TCCTGCCTGTGTCTATGACTAAGACTAGCGTTGATTGTACC |
| | ATGTATATTTGTGGCGACTCTACCGAATGTTCTAACCTGCT |
| | GCTTCAGTACGGCTCATTTTGCACACAGCTGAACAGAGCCC |
| | TGACTGGGATCGCTGTGGAGCAGGACAAGAACACACAGGA |
| | GGTGTTTGCACAGGTGAAGCAGATCTATAAGACCCCTCCTA |
| | TTAAGGATTTCGGCGGATTCAATTTCTCACAGATTCTGCCA |
| | GACCCCAGTAAGCCTTCCAAGAGGAGCTTCATCGAGGATCT |
| | CCTGTTTAACAAGGTGACCCTGGCAGACGCCGGCTTTATTA |
| | AGCAATATGGGGATTGCCTGGGCGACATTGCTGCCAGAGA |
| | CCTGATTTGCGCCCAGAAATTCAATGGCCTCACAGTGCTGC |
| | CACCTCTGCTGACCGACGAGATGATCGCTCAATACACTAGC |
| | GCACTGCTGGCCGGAACCATCACATCAGGCTGGACCTTCGG |
| | GGCCGGAGCAGCACTGCAGATTCCATTCGCCATGCAGATG |
| | GCCTATAGATTCAACGGCATTGGCGTCACACAGAACGTGCT |
| | GTACGAAAACCAGAAGCTCATCGCTAACCAGTTTAATTCCG |
| | CAATTGGAAAGATCCAAGATTCACTCAGCTCAACCGCCTCT |
| | GCACTCGGAAAGCTGCAGGACGTGGTCAACCAGAATGCTC |
| | AGGCCCTGAACACACTCGTCAAGCAGCTGTCCTCTAACTTT |
| | GGCGCTATCAGCTCCGTTCTGAACGACATTCTGAGCCGCCT |
| | GGATAAGGTGGAGGCTGAAGTCCAGATTGACCGCCTGATT |
| | ACCGGCCGGCTGCAGTCTCTGCAAACATACGTGACCCAGC |
| | AGCTGATCAGAGCAGCCGAGATCCGGGCATCCGCAAATCT |
| | GGCAGCAACTAAGATGAGCGAATGCGTGCTGGGCCAGTCC |
| | AAGCGGGTGGACTTTTGTGGCAAGGGCTACCACCTGATGA |
| | GCTTCCCCCAGAGCGCCCCACATGGCGTTGTTTTTCTGCAC |
| | GTGACCTATGTCCCTGCTCAGGAAAAGAACTTTACAACTGC |
| | TCCTGCTATCTGCCATGACGGCAAGGCCCACTTCCCACGGG |
| | AGGGAGTGTTTGTGTCCAATGGCACACACTGGTTCGTGACC |
| | CAGAGGAACTTCTATGAACCCCAGATCATCACCACTGACA |
| | ATACCTTCGTGTCTGGAAATTGCGACGTCGTGATCGGCATC |
| | GTTAACAACACCGTGTACGACCCTCTCCAGCCAGAGCTGGA |
| | CTCCTTTAAGGAGGAACTGGATAAGTATTTTAAGAACCACA |
| | CAAGCCCAGATGTGGATCTCGGGGACATCTCCGGAATTAA |
| | CGCCTCCGTGGTGAATATCCAGAAGGAGATTGACCGCCTA |
| | AATGAAGTTGCCAAGAACCTCAATGAGTCTCTGATTGATCT |
| | GCAGGAACTGGGCAAGTATGAGCAGGGGTACATTCCCGAG |
| | GCTCCTAGGGACGGCCAGGCATACGTGCGCAAAGACGGCG |
| | AGTGGGTGCTGCTGTCCACATTCCTGTGA |
| S2 subunit of a SARS- | (SEQ ID NO: 76) |
| CoV-2 S protein with a | MFVFLVLLPLVSSQCSVASQSIIAYTMSLGAENSVAYSNNSIAI |
| signal sequence and a | PTNFTISVTTEILPVSMTKTSVDCTMYICGDSTECSNLLLQYGS |
| Foldon | FCTQLNRALTGIAVEQDKNTQEVFAQVKQIYKTPPIKDFGGFN |
| | FSQILPDPSKPSKRSFIEDLLFNKVTLADAGFIKQYGDCLGDIA |
| | ARDLICAQKFNGLTVLPPLLTDEMIAQYTSALLAGTITSGWTF |
| | GAGAALQIPFAMQMAYRFNGIGVTQNVLYENQKLIANQFNSA |
| | IGKIQDSLSSTASALGKLQDVVNQNAQALNTLVKQLSSNFGAI |
| | SSVLNDILSRLDKVEAEVQIDRLITGRLQSLQTYVTQQLIRAAE |
| | IRASANLAATKMSECVLGQSKRVDFCGKGYHLMSFPQSAPHG |
| | VVFLHVTYVPAQEKNFTTAPAICHDGKAHFPREGVFVSNGTH |
| | WFVTQRNFYEPQIITTDNTFVSGNCDVVIGIVNNTVYDPLQPE |
| | LDSFKEELDKYFKNHTSPDVDLGDISGINASVVNIQKEIDRLNE |
| | VAKNLNESLIDLQELGKYEQGYIPEAPRDGQAYVRKDGEWVLL |
| | STFL |
| Optimized nucleotide | (SEQ ID NO: 77) |
| sequence encoding the | ATGTTCGTCTTCCTCGTGCTGCTCCCACTCGTTTCTTCCCAG |
| S2 subunit of a SARS- | TGTTCCGTTGCTAGTCAGTCTATTATTGCCTATACCATGAGC |
| CoV-2 S protein, which | CTCGGAGCTGAGAATAGCGTGGCCTACTCCAATAATTCCAT |
| has been modified to | CGCAATCCCTACTAACTTCACTATTTCTGTGACCACCGAGA |
| remove residues 986 and | TCCTGCCTGTGTCTATGACTAAGACTAGCGTTGATTGTACC |
| 987 with proline, with a | ATGTATATTTGTGGCGACTCTACCGAATGTTCTAACCTGCT |
| signal sequence | GCTTCAGTACGGCTCATTTTGCACACAGCTGAACAGAGCCC |
| | TGACTGGGATCGCTGTGGAGCAGGACAAGAACACACAGGA |
| | GGTGTTTGCACAGGTGAAGCAGATCTATAAGACCCCTCCTA |
| | TTAAGGATTTCGGCGGATTCAATTTCTCACAGATTCTGCCA |
| | GACCCCAGTAAGCCTTCCAAGAGGAGCTTCATCGAGGATCT |
| | CCTGTTTAACAAGGTGACCCTGGCAGACGCCGGCTTTATTA |
| | AGCAATATGGGGATTGCCTGGGCGACATTGCTGCCAGAGA |
| | CCTGATTTGCGCCCAGAAATTCAATGGCCTCACAGTGCTGC |
| | CACCTCTGCTGACCGACGAGATGATCGCTCAATACACTAGC |
| | GCACTGCTGGCCGGAACCATCACATCAGGCTGGACCTTCGG |
| | GGCCGGAGCAGCACTGCAGATTCCATTCGCCATGCAGATG |
| | GCCTATAGATTCAACGGCATTGGCGTCACACAGAACGTGCT |
| | GTACGAAAACCAGAAGCTCATCGCTAACCAGTTTAATTCCG |
| | CAATTGGAAAGATCCAAGATTCACTCAGCTCAACCGCCTCT |
| | GCACTCGGAAAGCTGCAGGACGTGGTCAACCAGAATGCTC |
| | AGGCCCTGAACACACTCGTCAAGCAGCTGTCCTCTAACTTT |
| | GGCGCTATCAGCTCCGTTCTGAACGACATTCTGAGCCGCCT |
| | GGATCCCCCAGAGGCTGAAGTCCAGATTGACCGCCTGATTA |
| | CCGGCCGGCTGCAGTCTCTGCAAACATACGTGACCCAGCA |
| | GCTGATCAGAGCAGCCGAGATCCGGGCATCCGCAAATCTG |
| | GCAGCAACTAAGATGAGCGAATGCGTGCTGGGCCAGTCCA |
| | AGCGGGTGGACTTTTGTGGCAAGGGCTACCACCTGATGAG |
| | CTTCCCCCAGAGCGCCCCACATGGCGTTGTTTTTCTGCACG |
| | TGACCTATGTCCCTGCTCAGGAAAAGAACTTTACAACTGCT |
| | CCTGCTATCTGCCATGACGGCAAGGCCCACTTCCCACGGGA |
| | GGGAGTGTTTGTGTCCAATGGCACACACTGGTTCGTGACCC |
| | AGAGGAACTTCTATGAACCCCAGATCATCACCACTGACAAT |
| | ACCTTCGTGTCTGGAAATTGCGACGTCGTGATCGGCATCGT |
| | TAACAACACCGTGTACGACCCTCTCCAGCCAGAGCTGGACT |
| | CCTTTAAGGAGGAACTGGATAAGTATTTTAAGAACCACAC |
| | AAGCCCAGATGTGGATCTCGGGGACATCTCCGGAATTAAC |
| | GCCTCCGTGGTGAATATCCAGAAGGAGATTGACCGCCTAA |
| | ATGAAGTTGCCAAGAACCTCAATGAGTCTCTGATTGATCTG |
| | CAGGAACTGGGCAAGTATGAGCAGTGA |
| S2 subunit of a SARS- | (SEQ ID NO: 78) |
| CoV-2 S protein which | MFVFLVLLPLVSSQCSVASQSIIAYTMSLGAENSVAYSNNSIAI |
| has been modified to | PTNFTISVTTEILPVSMTKTSVDCTMYICGDSTECSNLLLQYGS |
| remove residues 986 and | FCTQLNRALTGIAVEQDKNTQEVFAQVKQIYKTPPIKDFGGFN |
| 987 with proline | FSQILPDPSKPSKRSFIEDLLFNKVTLADAGFIKQYGDCLGDIA |
| | ARDLICAQKFNGLTVLPPLLTDEMIAQYTSALLAGTITSGWTF |
| | GAGAALQIPFAMQMAYRFNGIGVTQNVLYENQKLIANQFNSA |
| | IGKIQDSLSSTASALGKLQDVVNQNAQALNTLVKQLSSNFGAI |
| | SSVLNDILSRLDPPEAEVQIDRLITGRLQSLQTYVTQQLIRAAEI |
| | RASANLAATKMSECVLGQSKRVDFCGKGYHLMSFPQSAPHG |
| | VVFLHVTYVPAQEKNFTTAPAICHDGKAHFPREGVFVSNGTH |
| | WFVTQRNFYEPQIITTDNTFVSGNCDVVIGIVNNTVYDPLQPE |
| | LDSFKEELDKYFKNHTSPDVDLGDISGINASVVNIQKEIDRLNE |
| | VAKNLNESLIDLQELGKYEQ |
| Optimized nucleotide | (SEQ ID NO: 79) |
| sequence encoding S2 | ATGTTCGTCTTCCTCGTGCTGCTCCCACTCGTTTCTTCCCAG |
| subunit of a SARS-CoV- | TGTTCCGTTGCTAGTCAGTCTATTATTGCCTATACCATGAGC |
| 2 S protein with a signal | CTCGGAGCTGAGAATAGCGTGGCCTACTCCAATAATTCCAT |
| sequence, which has | CGCAATCCCTACTAACTTCACTATTTCTGTGACCACCGAGA |
| been modified to remove | TCCTGCCTGTGTCTATGACTAAGACTAGCGTTGATTGTACC |
| residues 986 and 987 | ATGTATATTTGTGGCGACTCTACCGAATGTTCTAACCTGCT |
| with proline, and a | GCTTCAGTACGGCTCATTTTGCACACAGCTGAACAGAGCCC |
| Foldon | TGACTGGGATCGCTGTGGAGCAGGACAAGAACACACAGGA |
| | GGTGTTTGCACAGGTGAAGCAGATCTATAAGACCCCTCCTA |
| | TTAAGGATTTCGGCGGATTCAATTTCTCACAGATTCTGCCA |
| | GACCCCAGTAAGCCTTCCAAGAGGAGCTTCATCGAGGATCT |
| | CCTGTTTAACAAGGTGACCCTGGCAGACGCCGGCTTTATTA |
| | AGCAATATGGGGATTGCCTGGGCGACATTGCTGCCAGAGA |
| | CCTGATTTGCGCCCAGAAATTCAATGGCCTCACAGTGCTGC |
| | CACCTCTGCTGACCGACGAGATGATCGCTCAATACACTAGC |
| | GCACTGCTGGCCGGAACCATCACATCAGGCTGGACCTTCGG |
| | GGCCGGAGCAGCACTGCAGATTCCATTCGCCATGCAGATG |
| | GCCTATAGATTCAACGGCATTGGCGTCACACAGAACGTGCT |
| | GTACGAAAACCAGAAGCTCATCGCTAACCAGTTTAATTCCG |
| | CAATTGGAAAGATCCAAGATTCACTCAGCTCAACCGCCTCT |
| | GCACTCGGAAAGCTGCAGGACGTGGTCAACCAGAATGCTC |
| | AGGCCCTGAACACACTCGTCAAGCAGCTGTCCTCTAACTTT |
| | GGCGCTATCAGCTCCGTTCTGAACGACATTCTGAGCCGCCT |
| | GGATCCCCCAGAGGCTGAAGTCCAGATTGACCGCCTGATTA |
| | CCGGCCGGCTGCAGTCTCTGCAAACATACGTGACCCAGCA |
| | GCTGATCAGAGCAGCCGAGATCCGGGCATCCGCAAATCTG |
| | GCAGCAACTAAGATGAGCGAATGCGTGCTGGGCCAGTCCA |
| | AGCGGGTGGACTTTTGTGGCAAGGGCTACCACCTGATGAG |
| | CTTCCCCCAGAGCGCCCCACATGGCGTTGTTTTTCTGCACG |
| | TGACCTATGTCCCTGCTCAGGAAAAGAACTTTACAACTGCT |
| | CCTGCTATCTGCCATGACGGCAAGGCCCACTTCCCACGGGA |
| | GGGAGTGTTTGTGTCCAATGGCACACACTGGTTCGTGACCC |
| | AGAGGAACTTCTATGAACCCCAGATCATCACCACTGACAAT |
| | ACCTTCGTGTCTGGAAATTGCGACGTCGTGATCGGCATCGT |
| | TAACAACACCGTGTACGACCCTCTCCAGCCAGAGCTGGACT |
| | CCTTTAAGGAGGAACTGGATAAGTATTTTAAGAACCACAC |
| | AAGCCCAGATGTGGATCTCGGGGACATCTCCGGAATTAAC |
| | GCCTCCGTGGTGAATATCCAGAAGGAGATTGACCGCCTAA |
| | ATGAAGTTGCCAAGAACCTCAATGAGTCTCTGATTGATCTG |
| | CAGGAACTGGGCAAGTATGAGCAGGGGTACATTCCCGAGG |
| | CTCCTAGGGACGGCCAGGCATACGTGCGCAAAGACGGCGA |
| | GTGGGTGCTGCTGTCCACATTCCTGTGA |
| S2 subunit of a SARS- | (SEQ ID NO: 80) |
| CoV-2 S protein, which | MFVFLVLLPLVSSQCSVASQSIIAYTMSLGAENSVAYSNNSIAI |
| has been modified to | PTNFTISVTTEILPVSMTKTSVDCTMYICGDSTECSNLLLQYGS |
| remove residues 986 and | FCTQLNRALTGIAVEQDKNTQEVFAQVKQIYKTPPIKDFGGFN |
| 987 with proline, with a | FSQILPDPSKPSKRSFIEDLLFNKVTLADAGFIKQYGDCLGDIA |
| signal sequence and a | ARDLICAQKFNGLTVLPPLLTDEMIAQYTSALLAGTITSGWTF |
| Foldon | GAGAALQIPFAMQMAYRFNGIGVTQNVLYENQKLIANQFNSA |
| | IGKIQDSLSSTASALGKLQDVVNQNAQALNTLVKQLSSNFGAI |
| | SSVLNDILSRLDPPEAEVQIDRLITGRLQSLQTYVTQQLIRAAEI |
| | RASANLAATKMSECVLGQSKRVDFCGKGYHLMSFPQSAPHG |
| | VVFLHVTYVPAQEKNFTTAPAICHDGKAHFPREGVFVSNGTH |
| | WFVTQRNFYEPQIITTDNTFVSGNCDVVIGIVNNTVYDPLQPE |
| | LDSFKEELDKYFKNHTSPDVDLGDISGINASVVNIQKEIDRLNE |
| | VAKNLNESLIDLQELGKYEQGYIPEAPRDGQAYVRKDGEWVLL |
| | STFL |
| Optimized nucleotide | (SEQ ID NO: 81) |
| sequence encoding the | ATGTTCGTCTTCCTCGTGCTGCTCCCACTCGTTTCTTCCCAG |
| S2′ subunit of a SARS- | TGTAGCTTCATCGAGGATCTCCTGTTTAACAAGGTGACCCT |
| CoV-2 S protein, which | GGCAGACGCCGGCTTTATTAAGCAATATGGGGATTGCCTGG |
| has been modified to | GCGACATTGCTGCCAGAGACCTGATTTGCGCCCAGAAATTC |
| remove residues 986 and | AATGGCCTCACAGTGCTGCCACCTCTGCTGACCGACGAGAT |
| 987 with proline, with a | GATCGCTCAATACACTAGCGCACTGCTGGCCGGAACCATCA |
| signal sequence | CATCAGGCTGGACCTTCGGGGCCGGAGCAGCACTGCAGAT |
| | TCCATTCGCCATGCAGATGGCCTATAGATTCAACGGCATTG |
| | GCGTCACACAGAACGTGCTGTACGAAAACCAGAAGCTCAT |
| | CGCTAACCAGTTTAATTCCGCAATTGGAAAGATCCAAGATT |
| | CACTCAGCTCAACCGCCTCTGCACTCGGAAAGCTGCAGGAC |
| | GTGGTCAACCAGAATGCTCAGGCCCTGAACACACTCGTCA |
| | AGCAGCTGTCCTCTAACTTTGGCGCTATCAGCTCCGTTCTG |
| | AACGACATTCTGAGCCGCCTGGATCCCCCAGAGGCTGAAG |
| | TCCAGATTGACCGCCTGATTACCGGCCGGCTGCAGTCTCTG |
| | CAAACATACGTGACCCAGCAGCTGATCAGAGCAGCCGAGA |
| | TCCGGGCATCCGCAAATCTGGCAGCAACTAAGATGAGCGA |
| | ATGCGTGCTGGGCCAGTCCAAGCGGGTGGACTTTTGTGGCA |
| | AGGGCTACCACCTGATGAGCTTCCCCCAGAGCGCCCCACAT |
| | GGCGTTGTTTTTCTGCACGTGACCTATGTCCCTGCTCAGGA |
| | AAAGAACTTTACAACTGCTCCTGCTATCTGCCATGACGGCA |
| | AGGCCCACTTCCCACGGGAGGGAGTGTTTGTGTCCAATGGC |
| | ACACACTGGTTCGTGACCCAGAGGAACTTCTATGAACCCCA |
| | GATCATCACCACTGACAATACCTTCGTGTCTGGAAATTGCG |
| | ACGTCGTGATCGGCATCGTTAACAACACCGTGTACGACCCT |
| | CTCCAGCCAGAGCTGGACTCCTTTAAGGAGGAACTGGATA |
| | AGTATTTTAAGAACCACACAAGCCCAGATGTGGATCTCGG |
| | GGACATCTCCGGAATTAACGCCTCCGTGGTGAATATCCAGA |
| | AGGAGATTGACCGCCTAAATGAAGTTGCCAAGAACCTCAA |
| | TGAGTCTCTGATTGATCTGCAGGAACTGGGCAAGTATGAGC |
| | AGTGA |
| S2′ subunit of a SARS- | (SEQ ID NO: 82) |
| CoV-2 S protein, which | MFVFLVLLPLVSSQCSFIEDLLFNKVTLADAGFIKQYGDCLGDI |
| has been modified to | AARDLICAQKFNGLTVLPPLLTDEMIAQYTSALLAGTITSGWT |
| remove residues 986 and | FGAGAALQIPFAMQMAYRFNGIGVTQNVLYENQKLIANQFNS |
| 987 with proline, with a | AIGKIQDSLSSTASALGKLQDVVNQNAQALNTLVKQLSSNFG |
| signal sequence | AISSVLNDILSRLDPPEAEVQIDRLITGRLQSLQTYVTQQLIRA |
| | AEIRASANLAATKMSECVLGQSKRVDFCGKGYHLMSFPQSAP |
| | HGVVFLHVTYVPAQEKNFTTAPAICHDGKAHFPREGVFVSNG |
| | THWFVTQRNFYEPQIITTDNTFVSGNCDVVIGIVNNTVYDPLQ |
| | PELDSFKEELDKYFKNHTSPDVDLGDISGINASVVNIQKEIDRL |
| | NEVAKNLNESLIDLQELGKYEQ |
| Optimized nucleotide | (SEQ ID NO: 83) |
| sequence encoding the | ATGTTCGTCTTCCTCGTGCTGCTCCCACTCGTTTCTTCCCAG |
| S2′ subunit of a SARS- | TGTAGCTTCATCGAGGATCTCCTGTTTAACAAGGTGACCCT |
| CoV-2 S protein, which | GGCAGACGCCGGCTTTATTAAGCAATATGGGGATTGCCTGG |
| has been modified to | GCGACATTGCTGCCAGAGACCTGATTTGCGCCCAGAAATTC |
| remove residues 986 and | AATGGCCTCACAGTGCTGCCACCTCTGCTGACCGACGAGAT |
| 987 with proline, with a | GATCGCTCAATACACTAGCGCACTGCTGGCCGGAACCATCA |
| signal sequence and a | CATCAGGCTGGACCTTCGGGGCCGGAGCAGCACTGCAGAT |
| Foldon | TCCATTCGCCATGCAGATGGCCTATAGATTCAACGGCATTG |
| | GCGTCACACAGAACGTGCTGTACGAAAACCAGAAGCTCAT |
| | CGCTAACCAGTTTAATTCCGCAATTGGAAAGATCCAAGATT |
| | CACTCAGCTCAACCGCCTCTGCACTCGGAAAGCTGCAGGAC |
| | GTGGTCAACCAGAATGCTCAGGCCCTGAACACACTCGTCA |
| | AGCAGCTGTCCTCTAACTTTGGCGCTATCAGCTCCGTTCTG |
| | AACGACATTCTGAGCCGCCTGGATCCCCCAGAGGCTGAAG |
| | TCCAGATTGACCGCCTGATTACCGGCCGGCTGCAGTCTCTG |
| | CAAACATACGTGACCCAGCAGCTGATCAGAGCAGCCGAGA |
| | TCCGGGCATCCGCAAATCTGGCAGCAACTAAGATGAGCGA |
| | ATGCGTGCTGGGCCAGTCCAAGCGGGTGGACTTTTGTGGCA |
| | AGGGCTACCACCTGATGAGCTTCCCCCAGAGCGCCCCACAT |
| | GGCGTTGTTTTTCTGCACGTGACCTATGTCCCTGCTCAGGA |
| | AAAGAACTTTACAACTGCTCCTGCTATCTGCCATGACGGCA |
| | AGGCCCACTTCCCACGGGAGGGAGTGTTTGTGTCCAATGGC |
| | ACACACTGGTTCGTGACCCAGAGGAACTTCTATGAACCCCA |
| | GATCATCACCACTGACAATACCTTCGTGTCTGGAAATTGCG |
| | ACGTCGTGATCGGCATCGTTAACAACACCGTGTACGACCCT |
| | CTCCAGCCAGAGCTGGACTCCTTTAAGGAGGAACTGGATA |
| | AGTATTTTAAGAACCACACAAGCCCAGATGTGGATCTCGG |
| | GGACATCTCCGGAATTAACGCCTCCGTGGTGAATATCCAGA |
| | AGGAGATTGACCGCCTAAATGAAGTTGCCAAGAACCTCAA |
| | TGAGTCTCTGATTGATCTGCAGGAACTGGGCAAGTATGAGC |
| | AGGGGTACATTCCCGAGGCTCCTAGGGACGGCCAGGCATA |
| | CGTGCGCAAAGACGGCGAGTGGGTGCTGCTGTCCACATTCC |
| | TGTGA |
| S2′ subunit of a SARS- | (SEQ ID NO: 84) |
| CoV-2 S protein, which | MFVFLVLLPLVSSQCSFIEDLLFNKVTLADAGFIKQYGDCLGDI |
| has been modified to | AARDLICAQKFNGLTVLPPLLTDEMIAQYTSALLAGTITSGWT |
| remove residues 986 and | FGAGAALQIPFAMQMAYRFNGIGVTQNVLYENQKLIANQFNS |
| 987 with proline, with a | AIGKIQDSLSSTASALGKLQDVVNQNAQALNTLVKQLSSNFG |
| signal sequence and a | AISSVLNDILSRLDPPEAEVQIDRLITGRLQSLQTYVTQQLIRA |
| Foldon | AEIRASANLAATKMSECVLGQSKRVDFCGKGYHLMSFPQSAP |
| | HGVVFLHVTYVPAQEKNFTTAPAICHDGKAHFPREGVFVSNG |
| | THWFVTQRNFYEPQIITTDNTFVSGNCDVVIGIVNNTVYDPLQ |
| | PELDSFKEELDKYFKNHTSPDVDLGDISGINASVVNIQKEIDRL |
| | NEVAKNLNESLIDLQELGKYEQGYIPEAPRDGQAYVRKDGEWV |
| | LLSTFL |
| Optimized nucleotide | (SEQ ID NO: 85) |
| sequence encoding the | ATGTTCGTCTTCCTCGTGCTGCTCCCACTCGTTTCTTCCCAG |
| full length S2′ subunit of | TGTAGCTTCATCGAGGATCTCCTGTTTAACAAGGTGACCCT |
| a SARS-CoV-2 S | GGCAGACGCCGGCTTTATTAAGCAATATGGGGATTGCCTGG |
| protein, which has been | GCGACATTGCTGCCAGAGACCTGATTTGCGCCCAGAAATTC |
| modified to remove | AATGGCCTCACAGTGCTGCCACCTCTGCTGACCGACGAGAT |
| residues 986 and 987 | GATCGCTCAATACACTAGCGCACTGCTGGCCGGAACCATCA |
| with proline, with a | CATCAGGCTGGACCTTCGGGGCCGGAGCAGCACTGCAGAT |
| signal sequence | TCCATTCGCCATGCAGATGGCCTATAGATTCAACGGCATTG |
| | GCGTCACACAGAACGTGCTGTACGAAAACCAGAAGCTCAT |
| | CGCTAACCAGTTTAATTCCGCAATTGGAAAGATCCAAGATT |
| | CACTCAGCTCAACCGCCTCTGCACTCGGAAAGCTGCAGGAC |
| | GTGGTCAACCAGAATGCTCAGGCCCTGAACACACTCGTCA |
| | AGCAGCTGTCCTCTAACTTTGGCGCTATCAGCTCCGTTCTG |
| | AACGACATTCTGAGCCGCCTGGATCCCCCAGAGGCTGAAG |
| | TCCAGATTGACCGCCTGATTACCGGCCGGCTGCAGTCTCTG |
| | CAAACATACGTGACCCAGCAGCTGATCAGAGCAGCCGAGA |
| | TCCGGGCATCCGCAAATCTGGCAGCAACTAAGATGAGCGA |
| | ATGCGTGCTGGGCCAGTCCAAGCGGGTGGACTTTTGTGGCA |
| | AGGGCTACCACCTGATGAGCTTCCCCCAGAGCGCCCCACAT |
| | GGCGTTGTTTTTCTGCACGTGACCTATGTCCCTGCTCAGGA |
| | AAAGAACTTTACAACTGCTCCTGCTATCTGCCATGACGGCA |
| | AGGCCCACTTCCCACGGGAGGGAGTGTTTGTGTCCAATGGC |
| | ACACACTGGTTCGTGACCCAGAGGAACTTCTATGAACCCCA |
| | GATCATCACCACTGACAATACCTTCGTGTCTGGAAATTGCG |
| | ACGTCGTGATCGGCATCGTTAACAACACCGTGTACGACCCT |
| | CTCCAGCCAGAGCTGGACTCCTTTAAGGAGGAACTGGATA |
| | AGTATTTTAAGAACCACACAAGCCCAGATGTGGATCTCGG |
| | GGACATCTCCGGAATTAACGCCTCCGTGGTGAATATCCAGA |
| | AGGAGATTGACCGCCTAAATGAAGTTGCCAAGAACCTCAA |
| | TGAGTCTCTGATTGATCTGCAGGAACTGGGCAAGTATGAGC |
| | AGTATATCAAATGGCCCTGGTACATTTGGCTGGGGTTTATC |
| | GCCGGACTGATTGCCATCGTCATGGTGACCATCATGCTGTG |
| | TTGCATGACCTCCTGTTGTTCCTGTCTGAAGGGCTGCTGTA |
| | GTTGCGGCTCTTGCTGTAAATTCGACGAAGATGATAGCGAG |
| | CCCGTGCTGAAGGGCGTGAAGCTGCATTATACCTGA |
| The full length S2′ | (SEQ ID NO: 86) |
| subunit of a SARS-CoV- | MFVFLVLLPLVSSQCSFIEDLLFNKVTLADAGFIKQYGDCLGDI |
| 2 S protein, which has | AARDLICAQKFNGLTVLPPLLTDEMIAQYTSALLAGTITSGWT |
| been modified to remove | FGAGAALQIPFAMQMAYRFNGIGVTQNVLYENQKLIANQFNS |
| residues 986 and 987 | AIGKIQDSLSSTASALGKLQDVVNQNAQALNTLVKQLSSNFG |
| with proline, with a | AISSVLNDILSRLDPPEAEVQIDRLITGRLQSLQTYVTQQLIRA |
| signal sequence | AEIRASANLAATKMSECVLGQSKRVDFCGKGYHLMSFPQSAP |
| | HGVVFLHVTYVPAQEKNFTTAPAICHDGKAHFPREGVFVSNG |
| | THWFVTQRNFYEPQIITTDNTFVSGNCDVVIGIVNNTVYDPLQ |
| | PELDSFKEELDKYFKNHTSPDVDLGDISGINASVVNIQKEIDRL |
| | NEVAKNLNESLIDLQELGKYEQYIKWPWYIWLGFIAGLIAIVM |
| | VTIMLCCMTSCCSCLKGCCSCGSCCKFDEDDSEPVLKGVKLH |
| | YT |
| Optimized nucleotide | (SEQ ID NO: 87) |
| sequence encoding of a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| which has been modified | CACCAACTCCTTCACCAGAGGCGTGTATTACCCAGACAAGG |
| to remove residues 985 | TGTTTAGAAGCAGCGTGCTGCACTCTACCCAGGACCTCTTT |
| with proline | CTGCCCTTTTTCAGCAACGTGACATGGTTTCACGCAATTCA |
| | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTAGAAGGGCCAGGTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCTT |
| | CATCGAGGATCTCCTGTTTAACAAGGTGACCCTGGCAGACG |
| | CCGGCTTTATTAAGCAATATGGGGATTGCCTGGGCGACATT |
| | GCTGCCAGAGACCTGATTTGCGCCCAGAAATTCAATGGCCT |
| | CACAGTGCTGCCACCTCTGCTGACCGACGAGATGATCGCTC |
| | AATACACTAGCGCACTGCTGGCCGGAACCATCACATCAGG |
| | CTGGACCTTCGGGGCCGGAGCAGCACTGCAGATTCCATTCG |
| | CCATGCAGATGGCCTATAGATTCAACGGCATTGGCGTCACA |
| | CAGAACGTGCTGTACGAAAACCAGAAGCTCATCGCTAACC |
| | AGTTTAATTCCGCAATTGGAAAGATCCAAGATTCACTCAGC |
| | TCAACCGCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGCCCAAGGTGGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGTATATC |
| | AAATGGCCCTGGTACATTTGGCTGGGGTTTATCGCCGGACT |
| | GATTGCCATCGTCATGGTGACCATCATGCTGTGTTGCATGA |
| | CCTCCTGTTGTTCCTGTCTGAAGGGCTGCTGTAGTTGCGGCT |
| | CTTGCTGTAAATTCGACGAAGATGATAGCGAGCCCGTGCTG |
| | AAGGGCGTGAAGCTGCATTATACCTGA |
| A SARS-CoV-2 S | (SEQ ID NO: 88) |
| protein which has been | MFVFLVLLPLVSSQCVNLTTRTQLPPAYTNSFTRGVYYPDKVF |
| modified to remove | RSSVLHSTQDLFLPFFSNVTWFHAIHVSGTNGTKRFDNPVLPF |
| residues 985 with | NDGVYFASTEKSNIIRGWIFGTTLDSKTQSLLIVNNATNVVIKV |
| proline | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPRRARSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLPKVEAEV |
| | QIDRLITGRLQSLQTYVTQQLIRAAEIRASANLAATKMSECVL |
| | GQSKRVDFCGKGYHLMSFPQSAPHGVVFLHVTYVPAQEKNF |
| | TTAPAICHDGKAHFPREGVFVSNGTHWFVTQRNFYEPQIITTD |
| | NTFVSGNCDVVIGIVNNTVYDPLQPELDSFKEELDKYFKNHTS |
| | PDVDLGDISGINASVVNIQKEIDRLNEVAKNLNESLIDLQELGK |
| | YEQYIKWPWYIWLGFIAGLIAIVMVTIMLCCMTSCCSCLKGCC |
| | SCGSCCKFDEDDSEPVLKGVKLHYT |
| Optimized nucleotide | (SEQ ID NO: 89) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| which has been modified | CACCAACTCCTTCACCAGAGGCGTGTATTACCCAGACAAGG |
| to remove a furin | TGTTTAGAAGCAGCGTGCTGCACTCTACCCAGGACCTCTTT |
| cleavage site and to | CTGCCCTTTTTCAGCAACGTGACATGGTTTCACGCAATTCA |
| replace residues 985 | CGTGTCCGGCACTAATGGCACAAAGCGGTTCGACAATCCA |
| with proline | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTGGCTCCGCCTCTTCCGTTGCTAGTCAGTCTATTATTGCC |
| | TATACCATGAGCCTCGGAGCTGAGAATAGCGTGGCCTACTC |
| | CAATAATTCCATCGCAATCCCTACTAACTTCACTATTTCTGT |
| | GACCACCGAGATCCTGCCTGTGTCTATGACTAAGACTAGCG |
| | TTGATTGTACCATGTATATTTGTGGCGACTCTACCGAATGTT |
| | CTAACCTGCTGCTTCAGTACGGCTCATTTTGCACACAGCTG |
| | AACAGAGCCCTGACTGGGATCGCTGTGGAGCAGGACAAGA |
| | ACACACAGGAGGTGTTTGCACAGGTGAAGCAGATCTATAA |
| | GACCCCTCCTATTAAGGATTTCGGCGGATTCAATTTCTCAC |
| | AGATTCTGCCAGACCCCAGTAAGCCTTCCAAGAGGAGCTTC |
| | ATCGAGGATCTCCTGTTTAACAAGGTGACCCTGGCAGACGC |
| | CGGCTTTATTAAGCAATATGGGGATTGCCTGGGCGACATTG |
| | CTGCCAGAGACCTGATTTGCGCCCAGAAATTCAATGGCCTC |
| | ACAGTGCTGCCACCTCTGCTGACCGACGAGATGATCGCTCA |
| | ATACACTAGCGCACTGCTGGCCGGAACCATCACATCAGGCT |
| | GGACCTTCGGGGCCGGAGCAGCACTGCAGATTCCATTCGCC |
| | ATGCAGATGGCCTATAGATTCAACGGCATTGGCGTCACACA |
| | GAACGTGCTGTACGAAAACCAGAAGCTCATCGCTAACCAG |
| | TTTAATTCCGCAATTGGAAAGATCCAAGATTCACTCAGCTC |
| | AACCGCCTCTGCACTCGGAAAGCTGCAGGACGTGGTCAAC |
| | CAGAATGCTCAGGCCCTGAACACACTCGTCAAGCAGCTGTC |
| | CTCTAACTTTGGCGCTATCAGCTCCGTTCTGAACGACATTCT |
| | GAGCCGCCTGCCCAAGGTGGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTGACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGTATATCAA |
| | ATGGCCCTGGTACATTTGGCTGGGGTTTATCGCCGGACTGA |
| | TTGCCATCGTCATGGTGACCATCATGCTGTGTTGCATGACC |
| | TCCTGTTGTTCCTGTCTGAAGGGCTGCTGTAGTTGCGGCTCT |
| | TGCTGTAAATTCGACGAAGATGATAGCGAGCCCGTGCTGA |
| | AGGGCGTGAAGCTGCATTATACCTGA |
| A SARS-CoV-2 S | (SEQ ID NO: 90) |
| protein which has been | MFVFLVLLPLVSSQCVNLTTRTQLPPAYTNSFTRGVYYPDKVF |
| modified to remove a | RSSVLHSTQDLFLPFFSNVTWFHAIHVSGTNGTKRFDNPVLPF |
| furin cleavage site and | NDGVYFASTEKSNIIRGWIFGTTLDSKTQSLLIVNNATNVVIKV |
| to replace residues 985 | CEFQFCNDPFLGVYYHKNNKSWMESEFRVYSSANNCTFEYVS |
| with proline | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLPKVEAEV |
| | QIDRLITGRLQSLQTYVTQQLIRAAEIRASANLAATKMSECVL |
| | GQSKRVDFCGKGYHLMSFPQSAPHGVVFLHVTYVPAQEKNF |
| | TTAPAICHDGKAHFPREGVFVSNGTHWFVTQRNFYEPQIITTD |
| | NTFVSGNCDVVIGIVNNTVYDPLQPELDSFKEELDKYFKNHTS |
| | PDVDLGDISGINASVVNIQKEIDRLNEVAKNLNESLIDLQELGK |
| | YEQYIKWPWYIWLGFIAGLIAIVMVTIMLCCMTSCCSCLKGCC |
| | SCGSCCKFDEDDSEPVLKGVKLHYT |
| Optimized nucleotide | (SEQ ID NO: 91) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| which has been modified | CACCAACTCCTTCACCAGAGGCGTGTATTACCCAGACAAGG |
| to replace residues 985, | TGTTTAGAAGCAGCGTGCTGCACTCTACCCAGGACCTCTTT |
| 986 and 987 with proline | CTGCCCTTTTTCAGCAACGTGACATGGTTTCACGCAATTCA |
| | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTAGAAGGGCCAGGTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCTT |
| | CATCGAGGATCTCCTGTTTAACAAGGTGACCCTGGCAGACG |
| | CCGGCTTTATTAAGCAATATGGGGATTGCCTGGGCGACATT |
| | GCTGCCAGAGACCTGATTTGCGCCCAGAAATTCAATGGCCT |
| | CACAGTGCTGCCACCTCTGCTGACCGACGAGATGATCGCTC |
| | AATACACTAGCGCACTGCTGGCCGGAACCATCACATCAGG |
| | CTGGACCTTCGGGGCCGGAGCAGCACTGCAGATTCCATTCG |
| | CCATGCAGATGGCCTATAGATTCAACGGCATTGGCGTCACA |
| | CAGAACGTGCTGTACGAAAACCAGAAGCTCATCGCTAACC |
| | AGTTTAATTCCGCAATTGGAAAGATCCAAGATTCACTCAGC |
| | TCAACCGCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGCCTCCACCCGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGTATATC |
| | AAATGGCCCTGGTACATTTGGCTGGGGTTTATCGCCGGACT |
| | GATTGCCATCGTCATGGTGACCATCATGCTGTGTTGCATGA |
| | CCTCCTGTTGTTCCTGTCTGAAGGGCTGCTGTAGTTGCGGCT |
| | CTTGCTGTAAATTCGACGAAGATGATAGCGAGCCCGTGCTG |
| | AAGGGCGTGAAGCTGCATTATACCTGA |
| A SARS-CoV-2 S | (SEQ ID NO: 92) |
| protein which has been | MFVFLVLLPLVSSQCVNLTTRTQLPPAYTNSFTRGVYYPDKVF |
| modified to replace | RSSVLHSTQDLFLPFFSNVTWFHAIHVSGTNGTKRFDNPVLPF |
| residues 985, 986 and | NDGVYFASTEKSNIIRGWIFGTTLDSKTQSLLIVNNATNVVIKV |
| 987 with proline | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPRRARSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLPPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 93) |
| encoding a SARS-CoV-2 | ATGTTCGTCTTCCTCGTGCTGCTCCCACTCGTTTCTTCCCAG |
| S protein sequence | TGTGTCAACCTGACAACTAGGACTCAGCTGCCACCAGCCTA |
| which has been modified | CACCAACTCCTTCACCAGAGGCGTGTATTACCCAGACAAGG |
| to remove a furin | TGTTTAGAAGCAGCGTGCTGCACTCTACCCAGGACCTCTTT |
| cleavage site and to | CTGCCCTTTTTCAGCAACGTGACATGGTTTCACGCAATTCA |
| replace residues 985, | CGTGTCCGGCACTAATGGCACAAAGCGGTTCGACAATCCA |
| 986 and 987 with proline | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTGGCTCCGCCTCTTCCGTTGCTAGTCAGTCTATTATTGCC |
| | TATACCATGAGCCTCGGAGCTGAGAATAGCGTGGCCTACTC |
| | CAATAATTCCATCGCAATCCCTACTAACTTCACTATTTCTGT |
| | GACCACCGAGATCCTGCCTGTGTCTATGACTAAGACTAGCG |
| | TTGATTGTACCATGTATATTTGTGGCGACTCTACCGAATGTT |
| | CTAACCTGCTGCTTCAGTACGGCTCATTTTGCACACAGCTG |
| | AACAGAGCCCTGACTGGGATCGCTGTGGAGCAGGACAAGA |
| | ACACACAGGAGGTGTTTGCACAGGTGAAGCAGATCTATAA |
| | GACCCCTCCTATTAAGGATTTCGGCGGATTCAATTTCTCAC |
| | AGATTCTGCCAGACCCCAGTAAGCCTTCCAAGAGGAGCTTC |
| | ATCGAGGATCTCCTGTTTAACAAGGTGACCCTGGCAGACGC |
| | CGGCTTTATTAAGCAATATGGGGATTGCCTGGGCGACATTG |
| | CTGCCAGAGACCTGATTTGCGCCCAGAAATTCAATGGCCTC |
| | ACAGTGCTGCCACCTCTGCTGACCGACGAGATGATCGCTCA |
| | ATACACTAGCGCACTGCTGGCCGGAACCATCACATCAGGCT |
| | GGACCTTCGGGGCCGGAGCAGCACTGCAGATTCCATTCGCC |
| | ATGCAGATGGCCTATAGATTCAACGGCATTGGCGTCACACA |
| | GAACGTGCTGTACGAAAACCAGAAGCTCATCGCTAACCAG |
| | TTTAATTCCGCAATTGGAAAGATCCAAGATTCACTCAGCTC |
| | AACCGCCTCTGCACTCGGAAAGCTGCAGGACGTGGTCAAC |
| | CAGAATGCTCAGGCCCTGAACACACTCGTCAAGCAGCTGTC |
| | CTCTAACTTTGGCGCTATCAGCTCCGTTCTGAACGACATTCT |
| | GAGCCGCCTGCCTCCACCCGAGGCTGAAGTCCAGATTGACC |
| | GCCTGATTACCGGCCGGCTGCAGTCTCTGCAAACATACGTG |
| | ACCCAGCAGCTGATCAGAGCAGCCGAGATCCGGGCATCCG |
| | CAAATCTGGCAGCAACTAAGATGAGCGAATGCGTGCTGGG |
| | CCAGTCCAAGCGGGTGGACTTTTGTGGCAAGGGCTACCACC |
| | TGATGAGCTTCCCCCAGAGCGCCCCACATGGCGTTGTTTTT |
| | CTGCACGTGACCTATGTCCCTGCTCAGGAAAAGAACTTTAC |
| | AACTGCTCCTGCTATCTGCCATGACGGCAAGGCCCACTTCC |
| | CACGGGAGGGAGTGTTTGTGTCCAATGGCACACACTGGTTC |
| | GTGACCCAGAGGAACTTCTATGAACCCCAGATCATCACCAC |
| | TGACAATACCTTCGTGTCTGGAAATTGCGACGTCGTGATCG |
| | GCATCGTTAACAACACCGTGTACGACCCTCTCCAGCCAGAG |
| | CTGGACTCCTTTAAGGAGGAACTGGATAAGTATTTTAAGAA |
| | CCACACAAGCCCAGATGTGGATCTCGGGGACATCTCCGGA |
| | ATTAACGCCTCCGTGGTGAATATCCAGAAGGAGATTGACC |
| | GCCTAAATGAAGTTGCCAAGAACCTCAATGAGTCTCTGATT |
| | GATCTGCAGGAACTGGGCAAGTATGAGCAGTATATCAAAT |
| | GGCCCTGGTACATTTGGCTGGGGTTTATCGCCGGACTGATT |
| | GCCATCGTCATGGTGACCATCATGCTGTGTTGCATGACCTC |
| | CTGTTGTTCCTGTCTGAAGGGCTGCTGTAGTTGCGGCTCTTG |
| | CTGTAAATTCGACGAAGATGATAGCGAGCCCGTGCTGAAG |
| | GGCGTGAAGCTGCATTATACCTGA |
| A SARS-CoV-2 S | (SEQ ID NO: 94) |
| protein which has been | MFVFLVLLPLVSSQCVNLTTRTQLPPAYTNSFTRGVYYPDKVF |
| modified to remove a | RSSVLHSTQDLFLPFFSNVTWFHAIHVSGTNGTKRFDNPVLPF |
| furin cleavage site and | NDGVYFASTEKSNIIRGWIFGTTLDSKTQSLLIVNNATNVVIKV |
| to replace residues 985, | CEFQFCNDPFLGVYYHKNNKSWMESEFRVYSSANNCTFEYVS |
| 986 and 987 with proline | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLPPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 95) |
| sequence encoding a | ATGTTCGTCTTCCTCGTGCTGCTCCCACTCGTTTCTTCCCAG |
| SARS-CoV-2 S2′ | TGTAGCTTCATCGAGGATCTCCTGTTTAACAAGGTGACCCT |
| subunit protein | GGCAGACGCCGGCTTTATTAAGCAATATGGGGATTGCCTGG |
| sequence with a signal | GCGACATTGCTGCCAGAGACCTGATTTGCGCCCAGAAATTC |
| sequence | AATGGCCTCACAGTGCTGCCACCTCTGCTGACCGACGAGAT |
| | GATCGCTCAATACACTAGCGCACTGCTGGCCGGAACCATCA |
| | CATCAGGCTGGACCTTCGGGGCCGGAGCAGCACTGCAGAT |
| | TCCATTCGCCATGCAGATGGCCTATAGATTCAACGGCATTG |
| | GCGTCACACAGAACGTGCTGTACGAAAACCAGAAGCTCAT |
| | CGCTAACCAGTTTAATTCCGCAATTGGAAAGATCCAAGATT |
| | CACTCAGCTCAACCGCCTCTGCACTCGGAAAGCTGCAGGAC |
| | GTGGTCAACCAGAATGCTCAGGCCCTGAACACACTCGTCA |
| | AGCAGCTGTCCTCTAACTTTGGCGCTATCAGCTCCGTTCTG |
| | AACGACATTCTGAGCCGCCTGGATAAGGTGGAGGCTGAAG |
| | TCCAGATTGACCGCCTGATTACCGGCCGGCTGCAGTCTCTG |
| | CAAACATACGTGACCCAGCAGCTGATCAGAGCAGCCGAGA |
| | TCCGGGCATCCGCAAATCTGGCAGCAACTAAGATGAGCGA |
| | ATGCGTGCTGGGCCAGTCCAAGCGGGTGGACTTTTGTGGCA |
| | AGGGCTACCACCTGATGAGCTTCCCCCAGAGCGCCCCACAT |
| | GGCGTTGTTTTTCTGCACGTGACCTATGTCCCTGCTCAGGA |
| | AAAGAACTTTACAACTGCTCCTGCTATCTGCCATGACGGCA |
| | AGGCCCACTTCCCACGGGAGGGAGTGTTTGTGTCCAATGGC |
| | ACACACTGGTTCGTGACCCAGAGGAACTTCTATGAACCCCA |
| | GATCATCACCACTGACAATACCTTCGTGTCTGGAAATTGCG |
| | ACGTCGTGATCGGCATCGTTAACAACACCGTGTACGACCCT |
| | CTCCAGCCAGAGCTGGACTCCTTTAAGGAGGAACTGGATA |
| | AGTATTTTAAGAACCACACAAGCCCAGATGTGGATCTCGG |
| | GGACATCTCCGGAATTAACGCCTCCGTGGTGAATATCCAGA |
| | AGGAGATTGACCGCCTAAATGAAGTTGCCAAGAACCTCAA |
| | TGAGTCTCTGATTGATCTGCAGGAACTGGGCAAGTATGAGC |
| | AGTATATCAAATGGCCCTGGTACATTTGGCTGGGGTTTATC |
| | GCCGGACTGATTGCCATCGTCATGGTGACCATCATGCTGTG |
| | TTGCATGACCTCCTGTTGTTCCTGTCTGAAGGGCTGCTGTA |
| | GTTGCGGCTCTTGCTGTAAATTCGACGAAGATGATAGCGAG |
| | CCCGTGCTGAAGGGCGTGAAGCTGCATTATACCTGA |
| Full length SARS-CoV-2 | (SEQ ID NO: 96) |
| S2′ subunit of a SARS | MFVFLVLLPLVSSQCSFIEDLLFNKVTLADAGFIKQYGDCLGDI |
| CoV-2 protein with a | AARDLICAQKFNGLTVLPPLLTDEMIAQYTSALLAGTITSGWT |
| signal sequence | FGAGAALQIPFAMQMAYRFNGIGVTQNVLYENQKLIANQFNS |
| | AIGKIQDSLSSTASALGKLQDVVNQNAQALNTLVKQLSSNFG |
| | AISSVLNDILSRLDKVEAEVQIDRLITGRLQSLQTYVTQQLIRA |
| | AEIRASANLAATKMSECVLGQSKRVDFCGKGYHLMSFPQSAP |
| | HGVVFLHVTYVPAQEKNFTTAPAICHDGKAHFPREGVFVSNG |
| | THWFVTQRNFYEPQIITTDNTFVSGNCDVVIGIVNNTVYDPLQ |
| | PELDSFKEELDKYFKNHTSPDVDLGDISGINASVVNIQKEIDRL |
| | NEVAKNLNESLIDLQELGKYEQYIKWPWYIWLGFIAGLIAIVM |
| | VTIMLCCMTSCCSCLKGCCSCGSCCKFDEDDSEPVLKGVKLH |
| | YT |
| Optimized nucleotide | (SEQ ID NO: 105) |
| sequence encoding a | ATGTTCGTCTTCCTCGTGCTGCTCCCACTCGTTTCTTCCCAG |
| receptor-binding | TGTCCTAATATCACTAACCTGTGTCCTTTCGGTGAAGTGTTC |
| domain of SARS-CoV-2 | AACGCCACCAGGTTTGCTAGCGTGTATGCCTGGAACAGGA |
| S protein with a signal | AGAGGATCTCTAACTGCGTCGCCGACTATTCCGTGCTGTAT |
| sequence and a mutated | AACAGCGCCTCCTTCTCCACATTCAAATGCTATGGAGTGAG |
| Fc region | CCCGACAAAACTGAACGATCTCTGCTTTACAAATGTCTACG |
| (L309D/Q311H/N434S) | CCGACTCTTTTGTGATCAGAGGGGACGAGGTCCGGCAGATC |
| | GCACCAGGACAGACAGGCAAGATTGCTGACTACAACTATA |
| | AGCTGCCTGACGACTTCACAGGATGTGTGATCGCATGGAAC |
| | TCAAACAATCTGGACTCCAAAGTCGGGGGCAACTATAATT |
| | ACCTGTATCGCCTGTTCCGGAAGTCCAACCTGAAGCCCTTC |
| | GAGAGGGACATCAGTACAGAGATCTATCAGGCTGGCTCCA |
| | CCCCTTGCAATGGCGTCGAAGGCTTTAATTGTTATTTTCCCC |
| | TGCAGTCTTACGGGTTTCAGCCTACTAATGGAGTTGGGTAC |
| | CAGCCATACAGAGTGGTCGTGCTCAGCTTCGAGCTCCTGCA |
| | TGCTCCACCTAAGTCCTGCGACAAAACCCATACATGTCCAC |
| | CATGCCCAGCTCCTGAACTGCTCGGCGGGCCTAGTGTTTTC |
| | CTCTTCCCTCCTAAGCCCAAGGATACCCTCATGATCTCTCG |
| | CACACCAGAAGTGACCTGCGTGGTCGTGGATGTCTCTCACG |
| | AGGATCCTGAAGTGAAGTTTAACTGGTATGTCGACGGAGT |
| | GGAAGTGCACAACGCCAAGACAAAGCCAAGAGAAGAACA |
| | ATACAATTCTACTTATAGGGTGGTGTCTGTGCTGACAGTGG |
| | ATCACCATGATTGGCTGAATGGAAAAGAATATAAGTGTAA |
| | GGTCTCTAACAAGGCCCTGCCCGCTCCAATTGAGAAGACA |
| | ATTTCCAAGGCCAAGGGGCAGCCTCGGGAACCTCAGGTGT |
| | ACACACTGCCCCCATCCAGGGATGAACTGACTAAAAATCA |
| | GGTGTCTCTGACATGCCTGGTGAAAGGGTTTTATCCAAGTG |
| | ACATTGCTGTGGAGTGGGAGTCTAATGGGCAGCCTGAAAA |
| | TAACTACAAGACCACACCACCAGTGCTCGATAGCGACGGG |
| | TCTTTCTTTCTGTATTCTAAACTGACCGTGGATAAATCTCGG |
| | TGGCAGCAGGGAAACGTGTTTTCTTGCTCAGTGATGCACGA |
| | AGCTCTGCACTCTCACTATACACAGAAATCCCTGTCCCTGT |
| | CTCCAGGCAAATAA |
| A receptor-binding | (SEQ ID NO: 104) |
| domain of SARS-CoV-2 | MFVFLVLLPLVSSQCPNITNLCPFGEVFNATRFASVYAWNRKR |
| S protein with a signal | ISNCVADYSVLYNSASFSTFKCYGVSPTKLNDLCFTNVYADSF |
| sequence and a mutated | VIRGDEVRQIAPGQTGKIADYNYKLPDDFTGCVIAWNSNNLD |
| Fc region | SKVGGNYNYLYRLFRKSNLKPFERDISTEIYQAGSTPCNGVEG |
| (L309D/Q311H/N434S) | FNCYFPLQSYGFQPTNGVGYQPYRVVVLSFELLHAPPKSCDKT |
| | HTCPPCPAPELLGGPSVFLFPPKPKDTLMISRTPEVTCVVVDVSH |
| | EDPEVKFNWYVDGVEVHNAKTKPREEQYNSTYRVVSVLTVDHHD |
| | WLNGKEYKCKVSNKALPAPIEKTISKAKGOPREPQVYTLPPSRDE |
| | LTKNQVSLTCLVKGFYPSDIAVEWESNGQPENNYKTTPPVLDSDG |
| | SFFLYSKLTVDKSRWQQGNVFSCSVMHEALHSHYTQKSLSLSPGK |
| Optimized nucleotide | (SEQ ID NO: 107) |
| sequence encoding a | ATGTTCGTCTTCCTCGTGCTGCTCCCACTCGTTTCTTCCCAG |
| receptor-binding | TGTCCTAATATCACTAACCTGTGTCCTTTCGGTGAAGTGTTC |
| domain of SARS-CoV-2 | AACGCCACCAGGTTTGCTAGCGTGTATGCCTGGAACAGGA |
| S protein with a signal | AGAGGATCTCTAACTGCGTCGCCGACTATTCCGTGCTGTAT |
| sequence and a mutated | AACAGCGCCTCCTTCTCCACATTCAAATGCTATGGAGTGAG |
| Fc region | CCCGACAAAACTGAACGATCTCTGCTTTACAAATGTCTACG |
| (M428L/N434S) | CCGACTCTTTTGTGATCAGAGGGGACGAGGTCCGGCAGATC |
| | GCACCAGGACAGACAGGCAAGATTGCTGACTACAACTATA |
| | AGCTGCCTGACGACTTCACAGGATGTGTGATCGCATGGAAC |
| | TCAAACAATCTGGACTCCAAAGTCGGGGGCAACTATAATT |
| | ACCTGTATCGCCTGTTCCGGAAGTCCAACCTGAAGCCCTTC |
| | GAGAGGGACATCAGTACAGAGATCTATCAGGCTGGCTCCA |
| | CCCCTTGCAATGGCGTCGAAGGCTTTAATTGTTATTTTCCCC |
| | TGCAGTCTTACGGGTTTCAGCCTACTAATGGAGTTGGGTAC |
| | CAGCCATACAGAGTGGTCGTGCTCAGCTTCGAGCTCCTGCA |
| | TGCTCCACCTAAGTCCTGCGACAAAACCCATACATGTCCAC |
| | CATGCCCAGCTCCTGAACTGCTCGGCGGGCCTAGTGTTTTC |
| | CTCTTCCCTCCTAAGCCCAAGGATACCCTCTATATCACTCG |
| | CGAACCAGAAGTGACCTGCGTGGTCGTGGATGTCTCTCACG |
| | AGGATCCTGAAGTGAAGTTTAACTGGTATGTCGACGGAGT |
| | GGAAGTGCACAACGCCAAGACAAAGCCAAGAGAAGAACA |
| | ATACAATTCTACTTATAGGGTGGTGTCTGTGCTGACAGTGC |
| | TGCACCAGGATTGGCTGAATGGAAAAGAATATAAGTGTAA |
| | GGTCTCTAACAAGGCCCTGCCCGCTCCAATTGAGAAGACA |
| | ATTTCCAAGGCCAAGGGGCAGCCTCGGGAACCTCAGGTGT |
| | ACACACTGCCCCCATCCAGGGATGAACTGACTAAAAATCA |
| | GGTGTCTCTGACATGCCTGGTGAAAGGGTTTTATCCAAGTG |
| | ACATTGCTGTGGAGTGGGAGTCTAATGGGCAGCCTGAAAA |
| | TAACTACAAGACCACACCACCAGTGCTCGATAGCGACGGG |
| | TCTTTCTTTCTGTATTCTAAACTGACCGTGGATAAATCTCGG |
| | TGGCAGCAGGGAAACGTGTTTTCTTGCTCAGTGATGCACGA |
| | AGCTCTGCACAATCACTATACACAGAAATCCCTGTCCCTGT |
| | CTCCAGGCAAATAA |
| A receptor-binding | (SEQ ID NO: 106) |
| domain of SARS-CoV-2 | MFVFLVLLPLVSSQCPNITNLCPFGEVFNATRFASVYAWNRKR |
| S protein with a signal | ISNCVADYSVLYNSASFSTFKCYGVSPTKLNDLCFTNVYADSF |
| sequence and a mutated | VIRGDEVRQIAPGQTGKIADYNYKLPDDFTGCVIAWNSNNLD |
| Fc region | SKVGGNYNYLYRLFRKSNLKPFERDISTEIYQAGSTPCNGVEG |
| (M428L/N434S) | FNCYFPLQSYGFQPTNGVGYQPYRVVVLSFELLHAPPKSCDKT |
| | HTCPPCPAPELLGGPSVFLFPPKPKDTLMISRTPEVTCVVVDVSH |
| | EDPEVKFNWYVDGVEVHNAKTKPREEQYNSTYRVVSVLTVLHQD |
| | WLNGKEYKCKVSNKALPAPIEKTISKAKGQPREPQVYTLPPSRDE |
| | LTKNQVSLTCLVKGFYPSDIAVEWESNGQPENNYKTTPPVLDSDG |
| | SFFLYSKLTVDKSRWQQGNVFSCSVLHEALHSHYTQKSLSLSPGK |
| Optimized nucleotide | (SEQ ID NO: 109) |
| sequence encoding a | ATGTTCGTCTTCCTCGTGCTGCTCCCACTCGTTTCTTCCCAG |
| receptor-binding | TGTCCTAATATCACTAACCTGTGTCCTTTCGGTGAAGTGTTC |
| domain of SARS-CoV-2 | AACGCCACCAGGTTTGCTAGCGTGTATGCCTGGAACAGGA |
| S protein with a signal | AGAGGATCTCTAACTGCGTCGCCGACTATTCCGTGCTGTAT |
| sequence and a mutated | AACAGCGCCTCCTTCTCCACATTCAAATGCTATGGAGTGAG |
| Fc region | CCCGACAAAACTGAACGATCTCTGCTTTACAAATGTCTACG |
| (M252Y/S254T/T256E) | CCGACTCTTTTGTGATCAGAGGGGACGAGGTCCGGCAGATC |
| | GCACCAGGACAGACAGGCAAGATTGCTGACTACAACTATA |
| | AGCTGCCTGACGACTTCACAGGATGTGTGATCGCATGGAAC |
| | TCAAACAATCTGGACTCCAAAGTCGGGGGCAACTATAATT |
| | ACCTGTATCGCCTGTTCCGGAAGTCCAACCTGAAGCCCTTC |
| | GAGAGGGACATCAGTACAGAGATCTATCAGGCTGGCTCCA |
| | CCCCTTGCAATGGCGTCGAAGGCTTTAATTGTTATTTTCCCC |
| | TGCAGTCTTACGGGTTTCAGCCTACTAATGGAGTTGGGTAC |
| | CAGCCATACAGAGTGGTCGTGCTCAGCTTCGAGCTCCTGCA |
| | TGCTCCACCTAAGTCCTGCGACAAAACCCATACATGTCCAC |
| | CATGCCCAGCTCCTGAACTGCTCGGCGGGCCTAGTGTTTTC |
| | CTCTTCCCTCCTAAGCCCAAGGATACCCTCTATATCACTCG |
| | CGAACCAGAAGTGACCTGCGTGGTCGTGGATGTCTCTCACG |
| | AGGATCCTGAAGTGAAGTTTAACTGGTATGTCGACGGAGT |
| | GGAAGTGCACAACGCCAAGACAAAGCCAAGAGAAGAACA |
| | ATACAATTCTACTTATAGGGTGGTGTCTGTGCTGACAGTGC |
| | TGCACCAGGATTGGCTGAATGGAAAAGAATATAAGTGTAA |
| | GGTCTCTAACAAGGCCCTGCCCGCTCCAATTGAGAAGACA |
| | ATTTCCAAGGCCAAGGGGCAGCCTCGGGAACCTCAGGTGT |
| | ACACACTGCCCCCATCCAGGGATGAACTGACTAAAAATCA |
| | GGTGTCTCTGACATGCCTGGTGAAAGGGTTTTATCCAAGTG |
| | ACATTGCTGTGGAGTGGGAGTCTAATGGGCAGCCTGAAAA |
| | TAACTACAAGACCACACCACCAGTGCTCGATAGCGACGGG |
| | TCTTTCTTTCTGTATTCTAAACTGACCGTGGATAAATCTCGG |
| | TGGCAGCAGGGAAACGTGTTTTCTTGCTCAGTGATGCACGA |
| | AGCTCTGCACAATCACTATACACAGAAATCCCTGTCCCTGT |
| | CTCCAGGCAAATAA |
| A receptor-binding | (SEQ ID NO: 108) |
| domain of SARS-CoV-2 | MFVFLVLLPLVSSQCPNITNLCPFGEVFNATRFASVYAWNRKR |
| S protein with a signal | ISNCVADYSVLYNSASFSTFKCYGVSPTKLNDLCFTNVYADSF |
| sequence and a mutated | VIRGDEVRQIAPGQTGKIADYNYKLPDDFTGCVIAWNSNNLD |
| Fc region | SKVGGNYNYLYRLFRKSNLKPFERDISTEIYQAGSTPCNGVEG |
| (M252Y/S254T/T256E) | FNCYFPLQSYGFQPTNGVGYQPYRVVVLSFELLHAPPKSCDKT |
| | HTCPPCPAPELLGGPSVFLFPPKPKDTLYITREPEVTCVVVDVSH |
| | EDPEVKFNWYVDGVEVHNAKTKPREEQYNSTYRVVSVLTVLHQD |
| | WLNGKEYKCKVSNKALPAPIEKTISKAKGQPREPQVYTLPPSRDE |
| | LTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| Optimized nucleotide | (SEQ ID NO: 111) |
| sequence encoding a | ATGTTCGTCTTCCTCGTGCTGCTCCCACTCGTTTCTTCCCAG |
| receptor-binding | TGTCCTAATATCACTAACCTGTGTCCTTTCGGTGAAGTGTTC |
| domain of SARS-CoV-2 | AACGCCACCAGGTTTGCTAGCGTGTATGCCTGGAACAGGA |
| S protein with a signal | AGAGGATCTCTAACTGCGTCGCCGACTATTCCGTGCTGTAT |
| sequence and a mutated | AACAGCGCCTCCTTCTCCACATTCAAATGCTATGGAGTGAG |
| Fc region | CCCGACAAAACTGAACGATCTCTGCTTTACAAATGTCTACG |
| (H433K/N434F) | CCGACTCTTTTGTGATCAGAGGGGACGAGGTCCGGCAGATC |
| | GCACCAGGACAGACAGGCAAGATTGCTGACTACAACTATA |
| | AGCTGCCTGACGACTTCACAGGATGTGTGATCGCATGGAAC |
| | TCAAACAATCTGGACTCCAAAGTCGGGGGCAACTATAATT |
| | ACCTGTATCGCCTGTTCCGGAAGTCCAACCTGAAGCCCTTC |
| | GAGAGGGACATCAGTACAGAGATCTATCAGGCTGGCTCCA |
| | CCCCTTGCAATGGCGTCGAAGGCTTTAATTGTTATTTTCCCC |
| | TGCAGTCTTACGGGTTTCAGCCTACTAATGGAGTTGGGTAC |
| | CAGCCATACAGAGTGGTCGTGCTCAGCTTCGAGCTCCTGCA |
| | TGCTCCACCTAAGTCCTGCGACAAAACCCATACATGTCCAC |
| | CATGCCCAGCTCCTGAACTGCTCGGCGGGCCTAGTGTTTTC |
| | CTCTTCCCTCCTAAGCCCAAGGATACCCTCATGATCTCTCG |
| | CACACCAGAAGTGACCTGCGTGGTCGTGGATGTCTCTCACG |
| | AGGATCCTGAAGTGAAGTTTAACTGGTATGTCGACGGAGT |
| | GGAAGTGCACAACGCCAAGACAAAGCCAAGAGAAGAACA |
| | ATACAATTCTACTTATAGGGTGGTGTCTGTGCTGACAGTGC |
| | TGCACCAGGATTGGCTGAATGGAAAAGAATATAAGTGTAA |
| | GGTCTCTAACAAGGCCCTGCCCGCTCCAATTGAGAAGACA |
| | ATTTCCAAGGCCAAGGGGCAGCCTCGGGAACCTCAGGTGT |
| | ACACACTGCCCCCATCCAGGGATGAACTGACTAAAAATCA |
| | GGTGTCTCTGACATGCCTGGTGAAAGGGTTTTATCCAAGTG |
| | ACATTGCTGTGGAGTGGGAGTCTAATGGGCAGCCTGAAAA |
| | TAACTACAAGACCACACCACCAGTGCTCGATAGCGACGGG |
| | TCTTTCTTTCTGTATTCTAAACTGACCGTGGATAAATCTCGG |
| | TGGCAGCAGGGAAACGTGTTTTCTTGCTCAGTGATGCACGA |
| | AGCTCTGAAATTTCACTATACACAGAAATCCCTGTCCCTGT |
| | CTCCAGGCAAATAA |
| A receptor-binding | (SEQ ID NO: 110) |
| domain of SARS-CoV-2 | MFVFLVLLPLVSSQCPNITNLCPFGEVFNATRFASVYAWNRKR |
| S protein with a signal | ISNCVADYSVLYNSASFSTFKCYGVSPTKLNDLCFTNVYADSF |
| sequence and a mutated | VIRGDEVRQIAPGQTGKIADYNYKLPDDFTGCVIAWNSNNLD |
| Fc region | SKVGGNYNYLYRLFRKSNLKPFERDISTEIYQAGSTPCNGVEG |
| (H433K/N434F) | FNCYFPLQSYGFQPTNGVGYQPYRVVVLSFELLHAPPKSCDKT |
| | HTCPPCPAPELLGGPSVFLFPPKPKDTLMISRTPEVTCVVVDVSH |
| | EDPEVKFNWYVDGVEVHNAKTKPREEQYNSTYRVVSVLTVLHQD |
| | WLNGKEYKCKVSNKALPAPIEKTISKAKGQPREPQVYTLPPSRDE |
| | LTKNQVSLTCLVKGFYPSDIAVEWESNGQPENNYKTTPPVLDSDG |
| | SFFLYSKLTVDKSRWQQGNVFSCSVMHEALKFHYTQKSLSLSPGK |
| SARS-CoV-2 S protein | (SEQ ID NO: 122) |
| mutated to remove a | ATGTTCCTGCTGACAACAAAAAGAACCATGTTTGTGTTCCT |
| furin cleavage site, to | GGTGCTGCTGCCTCTGGTGTCCTCACAGTGTGTCAACCTGA |
| replace residues 986 and | CAACAAGAACTCAGCTGCCACCAGCCTACACCAACTCCTTC |
| 987 with proline and | ACCAGAGGCGTGTATTACCCAGACAAGGTGTTTAGAAGCA |
| containing an extended | GCGTGCTGCACTCTACCCAGGACCTCTTTCTGCCCTTTTTCA |
| signal sequence | GCAACGTGACATGGTTTCACGCAATTCACGTGTCCGGCACT |
| | AATGGCACAAAGCGGTTCGACAATCCAGTCCTGCCTTTCAA |
| | CGATGGCGTCTACTTTGCATCTACTGAGAAATCCAATATCA |
| | TTAGGGGATGGATCTTCGGCACAACCCTGGATTCTAAGACC |
| | CAGAGCCTGCTGATCGTCAACAACGCCACAAACGTGGTCA |
| | TTAAGGTTTGCGAGTTTCAGTTCTGTAACGATCCTTTTCTGG |
| | GCGTGTATTATCATAAGAACAATAAGAGCTGGATGGAGTC |
| | CGAGTTTAGAGTGTATAGCTCTGCAAATAATTGTACCTTTG |
| | AGTACGTGAGCCAGCCCTTTCTGATGGACCTGGAGGGAAA |
| | ACAAGGAAACTTCAAAAACCTGCGGGAATTCGTTTTCAAA |
| | AACATCGACGGCTATTTCAAGATCTATAGCAAGCATACCCC |
| | AATCAACCTCGTGAGGGACCTCCCCCAGGGCTTTAGCGCAC |
| | TGGAGCCACTGGTTGACCTGCCTATCGGCATTAATATCACA |
| | AGATTTCAGACCCTGCTGGCACTGCATAGAAGCTATCTGAC |
| | CCCTGGAGACTCCTCTAGTGGGTGGACTGCCGGCGCCGCTG |
| | CCTACTATGTGGGCTATCTGCAGCCACGGACATTCCTGCTG |
| | AAATACAATGAGAACGGGACAATCACAGATGCTGTTGATT |
| | GCGCACTCGACCCCCTGTCCGAGACAAAGTGCACTCTCAAG |
| | AGCTTTACCGTCGAGAAGGGCATCTATCAGACCTCAAACTT |
| | CAGGGTGCAGCCCACAGAATCTATCGTGCGCTTCCCTAATA |
| | TCACTAACCTGTGTCCTTTCGGTGAAGTGTTCAACGCCACC |
| | AGGTTTGCTAGCGTGTATGCCTGGAACAGGAAGAGGATCT |
| | CTAACTGCGTCGCCGACTATTCCGTGCTGTATAACAGCGCC |
| | TCCTTCTCCACATTCAAATGCTATGGAGTGAGCCCGACAAA |
| | ACTGAACGATCTCTGCTTTACAAATGTCTACGCCGACTCTT |
| | TTGTGATCAGAGGGGACGAGGTCCGGCAGATCGCACCAGG |
| | ACAGACAGGCAAGATTGCTGACTACAACTATAAGCTGCCT |
| | GACGACTTCACAGGATGTGTGATCGCATGGAACTCAAACA |
| | ATCTGGACTCCAAAGTCGGGGGCAACTATAATTACCTGTAT |
| | CGCCTGTTCCGGAAGTCCAACCTGAAGCCCTTCGAGAGGG |
| | ACATCAGTACAGAGATCTATCAGGCTGGCTCCACCCCTTGC |
| | AATGGCGTCGAAGGCTTTAATTGTTATTTTCCCCTGCAGTCT |
| | TACGGGTTTCAGCCTACTAATGGAGTTGGGTACCAGCCATA |
| | CAGAGTGGTCGTGCTCAGCTTCGAGCTCCTGCATGCTCCAG |
| | CTACAGTTTGCGGGCCAAAGAAGTCCACTAACCTGGTGAA |
| | GAATAAGTGCGTCAACTTCAACTTTAACGGGCTCACCGGCA |
| | CCGGCGTGCTGACTGAGAGCAACAAGAAGTTTCTGCCATTT |
| | CAACAGTTTGGACGGGACATTGCCGACACCACCGATGCCG |
| | TTCGGGATCCACAGACCCTGGAAATTCTGGACATTACACCG |
| | TGCAGCTTCGGGGGCGTGAGCGTGATCACACCCGGAACCA |
| | ATACAAGCAACCAGGTTGCCGTCCTGTATCAGGATGTCAAT |
| | TGCACAGAAGTGCCAGTTGCTATCCACGCAGACCAGCTGA |
| | CTCCCACATGGCGGGTGTATAGCACCGGATCCAACGTGTTT |
| | CAGACCCGCGCCGGATGTCTCATTGGGGCCGAGCACGTGA |
| | ATAACAGCTACGAGTGCGACATCCCCATTGGCGCCGGCATT |
| | TGTGCGTCTTACCAGACTCAGACCAACTCTCCTGGCTCCGC |
| | CTCTTCCGTTGCTAGTCAGTCTATTATTGCCTATACCATGAG |
| | CCTCGGAGCTGAGAATAGCGTGGCCTACTCCAATAATTCCA |
| | TCGCAATCCCTACTAACTTCACTATTTCTGTGACCACCGAG |
| | ATCCTGCCTGTGTCTATGACTAAGACTAGCGTTGATTGTAC |
| | CATGTATATTTGTGGCGACTCTACCGAATGTTCTAACCTGC |
| | TGCTTCAGTACGGCTCATTTTGCACACAGCTGAACAGAGCC |
| | CTGACTGGGATCGCTGTGGAGCAGGACAAGAACACACAGG |
| | AGGTGTTTGCACAGGTGAAGCAGATCTATAAGACCCCTCCT |
| | ATTAAGGATTTCGGCGGATTCAATTTCTCACAGATTCTGCC |
| | AGACCCCAGTAAGCCTTCCAAGAGGAGCTTCATCGAGGAT |
| | CTCCTGTTTAACAAGGTGACCCTGGCAGACGCCGGCTTTAT |
| | TAAGCAATATGGGGATTGCCTGGGCGACATTGCTGCCAGA |
| | GACCTGATTTGCGCCCAGAAATTCAATGGCCTCACAGTGCT |
| | GCCACCTCTGCTGACCGACGAGATGATCGCTCAATACACTA |
| | GCGCACTGCTGGCCGGAACCATCACATCAGGCTGGACCTTC |
| | GGGGCCGGAGCAGCACTGCAGATTCCATTCGCCATGCAGA |
| | TGGCCTATAGATTCAACGGCATTGGCGTCACACAGAACGTG |
| | CTGTACGAAAACCAGAAGCTCATCGCTAACCAGTTTAATTC |
| | CGCAATTGGAAAGATCCAAGATTCACTCAGCTCAACCGCCT |
| | CTGCACTCGGAAAGCTGCAGGACGTGGTCAACCAGAATGC |
| | TCAGGCCCTGAACACACTCGTCAAGCAGCTGTCCTCTAACT |
| | TTGGCGCTATCAGCTCCGTTCTGAACGACATTCTGAGCCGC |
| | CTGGATCCCCCAGAGGCTGAAGTCCAGATTGACCGCCTGAT |
| | TACCGGCCGGCTGCAGTCTCTGCAAACATACGTGACCCAGC |
| | AGCTGATCAGAGCAGCCGAGATCCGGGCATCCGCAAATCT |
| | GGCAGCAACTAAGATGAGCGAATGCGTGCTGGGCCAGTCC |
| | AAGCGGGTGGACTTTTGTGGCAAGGGCTACCACCTGATGA |
| | GCTTCCCCCAGAGCGCCCCACATGGCGTTGTTTTTCTGCAC |
| | GTGACCTATGTCCCTGCTCAGGAAAAGAACTTTACAACTGC |
| | TCCTGCTATCTGCCATGACGGCAAGGCCCACTTCCCACGGG |
| | AGGGAGTGTTTGTGTCCAATGGCACACACTGGTTCGTGACC |
| | CAGAGGAACTTCTATGAACCCCAGATCATCACCACTGACA |
| | ATACCTTCGTGTCTGGAAATTGCGACGTCGTGATCGGCATC |
| | GTTAACAACACCGTGTACGACCCTCTCCAGCCAGAGCTGGA |
| | CTCCTTTAAGGAGGAACTGGATAAGTATTTTAAGAACCACA |
| | CAAGCCCAGATGTGGATCTCGGGGACATCTCCGGAATTAA |
| | CGCCTCCGTGGTGAATATCCAGAAGGAGATTGACCGCCTA |
| | AATGAAGTTGCCAAGAACCTCAATGAGTCTCTGATTGATCT |
| | GCAGGAACTGGGCAAGTATGAGCAGTATATCAAATGGCCC |
| | TGGTACATTTGGCTGGGGTTTATCGCCGGACTGATTGCCAT |
| | CGTCATGGTGACCATCATGCTGTGTTGCATGACCTCCTGTT |
| | GTTCCTGTCTGAAGGGCTGCTGTAGTTGCGGCTCTTGCTGT |
| | AAATTCGACGAAGATGATAGCGAGCCCGTGCTGAAGGGCG |
| | TGAAGCTGCATTATACCTGA |
| Optimized nucleotide | (SEQ ID NO: 123) |
| sequence encoding a | MFLLTTKRTMFVFLVLLPLVSSQCVNLTTRTQLPPAYTNSFTRGVYYPD |
| SARS-CoV-2 S protein | KVFRSSVLHSTQDLFLPFFSNVTWFHAIHVSGTNGTKRFDNPV |
| mutated to remove a | LPFNDGVYFASTEKSNIIRGWIFGTTLDSKTQSLLIVNNATNVV |
| furin cleavage site, to | IKVCEFQFCNDPFLGVYYHKNNKSWMESEFRVYSSANNCTFE |
| replace residues 986 and | YVSQPFLMDLEGKQGNFKNLREFVFKNIDGYFKIYSKHTPINL |
| 987 with proline and | VRDLPQGFSALEPLVDLPIGINITRFQTLLALHRSYLTPGDSSSG |
| which contains an | WTAGAAAYYVGYLQPRTFLLKYNENGTITDAVDCALDPLSET |
| extended signal | KCTLKSFTVEKGIYQTSNFRVQPTESIVRFPNITNLCPFGEVFN |
| sequence | ATRFASVYAWNRKRISNCVADYSVLYNSASFSTFKCYGVSPT |
| | KLNDLCFTNVYADSFVIRGDEVRQIAPGQTGKIADYNYKLPD |
| | DFTGCVIAWNSNNLDSKVGGNYNYLYRLFRKSNLKPFERDIS |
| | TEIYQAGSTPCNGVEGFNCYFPLQSYGFQPTNGVGYQPYRVV |
| | VLSFELLHAPATVCGPKKSTNLVKNKCVNFNFNGLTGTGVLT |
| | ESNKKFLPFQQFGRDIADTTDAVRDPQTLEILDITPCSFGGVSV |
| | ITPGTNTSNQVAVLYQDVNCTEVPVAIHADQLTPTWRVYSTG |
| | SNVFQTRAGCLIGAEHVNNSYECDIPIGAGICASYQTQTNSPGS |
| | ASSVASQSIIAYTMSLGAENSVAYSNNSIAIPTNFTISVTTEILPV |
| | SMTKTSVDCTMYICGDSTECSNLLLQYGSFCTQLNRALTGIAV |
| | EQDKNTQEVFAQVKQIYKTPPIKDFGGFNFSQILPDPSKPSKRS |
| | FIEDLLFNKVTLADAGFIKQYGDCLGDIAARDLICAQKFNGLT |
| | VLPPLLTDEMIAQYTSALLAGTITSGWTFGAGAALQIPFAMQ |
| | MAYRFNGIGVTQNVLYENQKLIANQFNSAIGKIQDSLSSTASA |
| | LGKLQDVVNQNAQALNTLVKQLSSNFGAISSVLNDILSRLDPP |
| | EAEVQIDRLITGRLQSLQTYVTQQLIRAAEIRASANLAATKMS |
| | ECVLGQSKRVDFCGKGYHLMSFPQSAPHGVVFLHVTYVPAQ |
| | EKNFTTAPAICHDGKAHFPREGVFVSNGTHWFVTQRNFYEPQI |
| | ITTDNTFVSGNCDVVIGIVNNTVYDPLQPELDSFKEELDKYFK |
| | NHTSPDVDLGDISGINASVVNIQKEIDRLNEVAKNLNESLIDLQ |
| | ELGKYEQYIKWPWYIWLGFIAGLIAIVMVTIMLCCMTSCCSCL |
| | KGCCSCGSCCKFDEDDSEPVLKGVKLHYT |
| Optimized nucleotide | (SEQ ID NO: 124) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove a | CACCAACTCCTTCACCAGAGGCGTGTATTACCCAGACAAGG |
| furin cleavage site, to | TGTTTAGAAGCAGCGTGCTGCACTCTACCCAGGACCTCTTT |
| replace residues 986 and | CTGCCCTTTTTCAGCAACGTGACATGGTTTCACGCAATTCA |
| 987 with proline and to | CGTGTCCGGCACTAATGGCACAAAGCGGTTCGACAATCCA |
| mutate the ER retrieval | GTCCTGCCTTTCAACGATGGCGTCTACTTTGCATCTACTGA |
| signal | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTGGCTCCGCCTCTTCCGTTGCTAGTCAGTCTATTATTGCC |
| | TATACCATGAGCCTCGGAGCTGAGAATAGCGTGGCCTACTC |
| | CAATAATTCCATCGCAATCCCTACTAACTTCACTATTTCTGT |
| | GACCACCGAGATCCTGCCTGTGTCTATGACTAAGACTAGCG |
| | TTGATTGTACCATGTATATTTGTGGCGACTCTACCGAATGTT |
| | CTAACCTGCTGCTTCAGTACGGCTCATTTTGCACACAGCTG |
| | AACAGAGCCCTGACTGGGATCGCTGTGGAGCAGGACAAGA |
| | ACACACAGGAGGTGTTTGCACAGGTGAAGCAGATCTATAA |
| | GACCCCTCCTATTAAGGATTTCGGCGGATTCAATTTCTCAC |
| | AGATTCTGCCAGACCCCAGTAAGCCTTCCAAGAGGAGCTTC |
| | ATCGAGGATCTCCTGTTTAACAAGGTGACCCTGGCAGACGC |
| | CGGCTTTATTAAGCAATATGGGGATTGCCTGGGCGACATTG |
| | CTGCCAGAGACCTGATTTGCGCCCAGAAATTCAATGGCCTC |
| | ACAGTGCTGCCACCTCTGCTGACCGACGAGATGATCGCTCA |
| | ATACACTAGCGCACTGCTGGCCGGAACCATCACATCAGGCT |
| | GGACCTTCGGGGCCGGAGCAGCACTGCAGATTCCATTCGCC |
| | ATGCAGATGGCCTATAGATTCAACGGCATTGGCGTCACACA |
| | GAACGTGCTGTACGAAAACCAGAAGCTCATCGCTAACCAG |
| | TTTAATTCCGCAATTGGAAAGATCCAAGATTCACTCAGCTC |
| | AACCGCCTCTGCACTCGGAAAGCTGCAGGACGTGGTCAAC |
| | CAGAATGCTCAGGCCCTGAACACACTCGTCAAGCAGCTGTC |
| | CTCTAACTTTGGCGCTATCAGCTCCGTTCTGAACGACATTCT |
| | GAGCCGCCTGGATCCCCCAGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTGACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGTATATCAA |
| | ATGGCCCTGGTACATTTGGCTGGGGTTTATCGCCGGACTGA |
| | TTGCCATCGTCATGGTGACCATCATGCTGTGTTGCATGACC |
| | TCCTGTTGTTCCTGTCTGAAGGGCTGCTGTAGTTGCGGCTCT |
| | TGCTGTAAATTCGACGAAGATGATAGCGAGCCCGTGCTGA |
| | AGGGCGTGGCCCTGGCTTATACCTGA |
| SARS-CoV-2 S protein | (SEQ ID NO: 125) |
| mutated to remove a | MFVFLVLLPLVSSQCVNLTTRTQLPPAYTNSFTRGVYYPDKVF |
| furin cleavage site, to | RSSVLHSTQDLFLPFFSNVTWFHAIHVSGTNGTKRFDNPVLPF |
| replace residues 986 and | NDGVYFASTEKSNIIRGWIFGTTLDSKTQSLLIVNNATNVVIKV |
| 987 with proline and to | CEFQFCNDPFLGVYYHKNNKSWMESEFRVYSSANNCTFEYVS |
| mutate the ER retrieval | QPFLMDLEGKQGNFKNLREFVFKNIDGYFKIYSKHTPINLVRD |
| signal | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVALAYT |
| Optimized nucleotide | (SEQ ID NO: 126) |
| sequence encoding a | ATGTTCCTGCTGACAACAAAAAGAACCATGTTTGTGTTCCT |
| SARS-CoV-2 S protein | GGTGCTGCTGCCTCTGGTGTCCTCACAGTGTGTCAACCTGA |
| mutated to remove a | CAACAAGAACTCAGCTGCCACCAGCCTACACCAACTCCTTC |
| furin cleavage site, to | ACCAGAGGCGTGTATTACCCAGACAAGGTGTTTAGAAGCA |
| replace residues 986 and | GCGTGCTGCACTCTACCCAGGACCTCTTTCTGCCCTTTTTCA |
| 987 with proline, to | GCAACGTGACATGGTTTCACGCAATTCACGTGTCCGGCACT |
| mutate the ER retrieval | AATGGCACAAAGCGGTTCGACAATCCAGTCCTGCCTTTCAA |
| signal and which | CGATGGCGTCTACTTTGCATCTACTGAGAAATCCAATATCA |
| contains an extended | TTAGGGGATGGATCTTCGGCACAACCCTGGATTCTAAGACC |
| signal sequence | CAGAGCCTGCTGATCGTCAACAACGCCACAAACGTGGTCA |
| | TTAAGGTTTGCGAGTTTCAGTTCTGTAACGATCCTTTTCTGG |
| | GCGTGTATTATCATAAGAACAATAAGAGCTGGATGGAGTC |
| | CGAGTTTAGAGTGTATAGCTCTGCAAATAATTGTACCTTTG |
| | AGTACGTGAGCCAGCCCTTTCTGATGGACCTGGAGGGAAA |
| | ACAAGGAAACTTCAAAAACCTGCGGGAATTCGTTTTCAAA |
| | AACATCGACGGCTATTTCAAGATCTATAGCAAGCATACCCC |
| | AATCAACCTCGTGAGGGACCTCCCCCAGGGCTTTAGCGCAC |
| | TGGAGCCACTGGTTGACCTGCCTATCGGCATTAATATCACA |
| | AGATTTCAGACCCTGCTGGCACTGCATAGAAGCTATCTGAC |
| | CCCTGGAGACTCCTCTAGTGGGTGGACTGCCGGCGCCGCTG |
| | CCTACTATGTGGGCTATCTGCAGCCACGGACATTCCTGCTG |
| | AAATACAATGAGAACGGGACAATCACAGATGCTGTTGATT |
| | GCGCACTCGACCCCCTGTCCGAGACAAAGTGCACTCTCAAG |
| | AGCTTTACCGTCGAGAAGGGCATCTATCAGACCTCAAACTT |
| | CAGGGTGCAGCCCACAGAATCTATCGTGCGCTTCCCTAATA |
| | TCACTAACCTGTGTCCTTTCGGTGAAGTGTTCAACGCCACC |
| | AGGTTTGCTAGCGTGTATGCCTGGAACAGGAAGAGGATCT |
| | CTAACTGCGTCGCCGACTATTCCGTGCTGTATAACAGCGCC |
| | TCCTTCTCCACATTCAAATGCTATGGAGTGAGCCCGACAAA |
| | ACTGAACGATCTCTGCTTTACAAATGTCTACGCCGACTCTT |
| | TTGTGATCAGAGGGGACGAGGTCCGGCAGATCGCACCAGG |
| | ACAGACAGGCAAGATTGCTGACTACAACTATAAGCTGCCT |
| | GACGACTTCACAGGATGTGTGATCGCATGGAACTCAAACA |
| | ATCTGGACTCCAAAGTCGGGGGCAACTATAATTACCTGTAT |
| | CGCCTGTTCCGGAAGTCCAACCTGAAGCCCTTCGAGAGGG |
| | ACATCAGTACAGAGATCTATCAGGCTGGCTCCACCCCTTGC |
| | AATGGCGTCGAAGGCTTTAATTGTTATTTTCCCCTGCAGTCT |
| | TACGGGTTTCAGCCTACTAATGGAGTTGGGTACCAGCCATA |
| | CAGAGTGGTCGTGCTCAGCTTCGAGCTCCTGCATGCTCCAG |
| | CTACAGTTTGCGGGCCAAAGAAGTCCACTAACCTGGTGAA |
| | GAATAAGTGCGTCAACTTCAACTTTAACGGGCTCACCGGCA |
| | CCGGCGTGCTGACTGAGAGCAACAAGAAGTTTCTGCCATTT |
| | CAACAGTTTGGACGGGACATTGCCGACACCACCGATGCCG |
| | TTCGGGATCCACAGACCCTGGAAATTCTGGACATTACACCG |
| | TGCAGCTTCGGGGGCGTGAGCGTGATCACACCCGGAACCA |
| | ATACAAGCAACCAGGTTGCCGTCCTGTATCAGGATGTCAAT |
| | TGCACAGAAGTGCCAGTTGCTATCCACGCAGACCAGCTGA |
| | CTCCCACATGGCGGGTGTATAGCACCGGATCCAACGTGTTT |
| | CAGACCCGCGCCGGATGTCTCATTGGGGCCGAGCACGTGA |
| | ATAACAGCTACGAGTGCGACATCCCCATTGGCGCCGGCATT |
| | TGTGCGTCTTACCAGACTCAGACCAACTCTCCTGGCTCCGC |
| | CTCTTCCGTTGCTAGTCAGTCTATTATTGCCTATACCATGAG |
| | CCTCGGAGCTGAGAATAGCGTGGCCTACTCCAATAATTCCA |
| | TCGCAATCCCTACTAACTTCACTATTTCTGTGACCACCGAG |
| | ATCCTGCCTGTGTCTATGACTAAGACTAGCGTTGATTGTAC |
| | CATGTATATTTGTGGCGACTCTACCGAATGTTCTAACCTGC |
| | TGCTTCAGTACGGCTCATTTTGCACACAGCTGAACAGAGCC |
| | CTGACTGGGATCGCTGTGGAGCAGGACAAGAACACACAGG |
| | AGGTGTTTGCACAGGTGAAGCAGATCTATAAGACCCCTCCT |
| | ATTAAGGATTTCGGCGGATTCAATTTCTCACAGATTCTGCC |
| | AGACCCCAGTAAGCCTTCCAAGAGGAGCTTCATCGAGGAT |
| | CTCCTGTTTAACAAGGTGACCCTGGCAGACGCCGGCTTTAT |
| | TAAGCAATATGGGGATTGCCTGGGCGACATTGCTGCCAGA |
| | GACCTGATTTGCGCCCAGAAATTCAATGGCCTCACAGTGCT |
| | GCCACCTCTGCTGACCGACGAGATGATCGCTCAATACACTA |
| | GCGCACTGCTGGCCGGAACCATCACATCAGGCTGGACCTTC |
| | GGGGCCGGAGCAGCACTGCAGATTCCATTCGCCATGCAGA |
| | TGGCCTATAGATTCAACGGCATTGGCGTCACACAGAACGTG |
| | CTGTACGAAAACCAGAAGCTCATCGCTAACCAGTTTAATTC |
| | CGCAATTGGAAAGATCCAAGATTCACTCAGCTCAACCGCCT |
| | CTGCACTCGGAAAGCTGCAGGACGTGGTCAACCAGAATGC |
| | TCAGGCCCTGAACACACTCGTCAAGCAGCTGTCCTCTAACT |
| | TTGGCGCTATCAGCTCCGTTCTGAACGACATTCTGAGCCGC |
| | CTGGATCCCCCAGAGGCTGAAGTCCAGATTGACCGCCTGAT |
| | TACCGGCCGGCTGCAGTCTCTGCAAACATACGTGACCCAGC |
| | AGCTGATCAGAGCAGCCGAGATCCGGGCATCCGCAAATCT |
| | GGCAGCAACTAAGATGAGCGAATGCGTGCTGGGCCAGTCC |
| | AAGCGGGTGGACTTTTGTGGCAAGGGCTACCACCTGATGA |
| | GCTTCCCCCAGAGCGCCCCACATGGCGTTGTTTTTCTGCAC |
| | GTGACCTATGTCCCTGCTCAGGAAAAGAACTTTACAACTGC |
| | TCCTGCTATCTGCCATGACGGCAAGGCCCACTTCCCACGGG |
| | AGGGAGTGTTTGTGTCCAATGGCACACACTGGTTCGTGACC |
| | CAGAGGAACTTCTATGAACCCCAGATCATCACCACTGACA |
| | ATACCTTCGTGTCTGGAAATTGCGACGTCGTGATCGGCATC |
| | GTTAACAACACCGTGTACGACCCTCTCCAGCCAGAGCTGGA |
| | CTCCTTTAAGGAGGAACTGGATAAGTATTTTAAGAACCACA |
| | CAAGCCCAGATGTGGATCTCGGGGACATCTCCGGAATTAA |
| | CGCCTCCGTGGTGAATATCCAGAAGGAGATTGACCGCCTA |
| | AATGAAGTTGCCAAGAACCTCAATGAGTCTCTGATTGATCT |
| | GCAGGAACTGGGCAAGTATGAGCAGTATATCAAATGGCCC |
| | TGGTACATTTGGCTGGGGTTTATCGCCGGACTGATTGCCAT |
| | CGTCATGGTGACCATCATGCTGTGTTGCATGACCTCCTGTT |
| | GTTCCTGTCTGAAGGGCTGCTGTAGTTGCGGCTCTTGCTGT |
| | AAATTCGACGAAGATGATAGCGAGCCCGTGCTGAAGGGCG |
| | TGGCCCTGGCTTATACCTGA |
| SARS-CoV-2 S protein | (SEQ ID NO: 127) |
| mutated to remove a | MFLLTTKRTMFVFLVLLPLVSSQCVNLTTRTQLPPAYTNSFTRGVYYPD |
| furin cleavage site, to | KVFRSSVLHSTQDLFLPFFSNVTWFHAIHVSGTNGTKRFDNPV |
| replace residues 986 and | LPFNDGVYFASTEKSNIIRGWIFGTTLDSKTQSLLIVNNATNVV |
| 987 with proline, to | IKVCEFQFCNDPFLGVYYHKNNKSWMESEFRVYSSANNCTFE |
| mutate the ER retrieval | YVSQPFLMDLEGKQGNFKNLREFVFKNIDGYFKIYSKHTPINL |
| signal and which | VRDLPQGFSALEPLVDLPIGINITRFQTLLALHRSYLTPGDSSSG |
| contains an extended | WTAGAAAYYVGYLQPRTFLLKYNENGTITDAVDCALDPLSET |
| signal sequence | KCTLKSFTVEKGIYQTSNFRVQPTESIVRFPNITNLCPFGEVFN |
| | ATRFASVYAWNRKRISNCVADYSVLYNSASFSTFKCYGVSPT |
| | KLNDLCFTNVYADSFVIRGDEVRQIAPGQTGKIADYNYKLPD |
| | DFTGCVIAWNSNNLDSKVGGNYNYLYRLFRKSNLKPFERDIS |
| | TEIYQAGSTPCNGVEGFNCYFPLQSYGFQPTNGVGYQPYRVV |
| | VLSFELLHAPATVCGPKKSTNLVKNKCVNFNFNGLTGTGVLT |
| | ESNKKFLPFQQFGRDIADTTDAVRDPQTLEILDITPCSFGGVSV |
| | ITPGTNTSNQVAVLYQDVNCTEVPVAIHADQLTPTWRVYSTG |
| | SNVFQTRAGCLIGAEHVNNSYECDIPIGAGICASYQTQTNSPGS |
| | ASSVASQSIIAYTMSLGAENSVAYSNNSIAIPTNFTISVTTEILPV |
| | SMTKTSVDCTMYICGDSTECSNLLLQYGSFCTQLNRALTGIAV |
| | EQDKNTQEVFAQVKQIYKTPPIKDFGGFNFSQILPDPSKPSKRS |
| | FIEDLLFNKVTLADAGFIKQYGDCLGDIAARDLICAQKFNGLT |
| | VLPPLLTDEMIAQYTSALLAGTITSGWTFGAGAALQIPFAMQ |
| | MAYRFNGIGVTQNVLYENQKLIANQFNSAIGKIQDSLSSTASA |
| | LGKLQDVVNQNAQALNTLVKQLSSNFGAISSVLNDILSRLDPP |
| | EAEVQIDRLITGRLQSLQTYVTQQLIRAAEIRASANLAATKMS |
| | ECVLGQSKRVDFCGKGYHLMSFPQSAPHGVVFLHVTYVPAQ |
| | EKNFTTAPAICHDGKAHFPREGVFVSNGTHWFVTQRNFYEPQI |
| | ITTDNTFVSGNCDVVIGIVNNTVYDPLQPELDSFKEELDKYFK |
| | NHTSPDVDLGDISGINASVVNIQKEIDRLNEVAKNLNESLIDLQ |
| | ELGKYEQYIKWPWYIWLGFIAGLIAIVMVTIMLCCMTSCCSCL |
| | KGCCSCGSCCKFDEDDSEPVLKGVALAYT |
| Optimized nucleotide | (SEQ ID NO: 128) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to replace | CACCAACTCCTTCACCAGAGGCGTGTATTACCCAGACAAGG |
| residues 817, 892, 899, | TGTTTAGAAGCAGCGTGCTGCACTCTACCCAGGACCTCTTT |
| 942, 986 and 987 with | CTGCCCTTTTTCAGCAACGTGACATGGTTTCACGCAATTCA |
| proline | CGTGTCCGGCACTAATGGCACAAAGCGGTTCGACAATCCA |
| | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTAGAAGGGCCAGGTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCC |
| | CTATCGAGGATCTCCTGTTTAACAAGGTGACCCTGGCAGAC |
| | GCCGGCTTTATTAAGCAATATGGGGATTGCCTGGGCGACAT |
| | TGCTGCCAGAGACCTGATTTGCGCCCAGAAATTCAATGGCC |
| | TCACAGTGCTGCCACCTCTGCTGACCGACGAGATGATCGCT |
| | CAATACACTAGCGCACTGCTGGCCGGAACCATCACATCAG |
| | GCTGGACCTTCGGGGCCGGACCAGCACTGCAGATTCCATTC |
| | CCTATGCAGATGGCCTATAGATTCAACGGCATTGGCGTCAC |
| | ACAGAACGTGCTGTACGAAAACCAGAAGCTCATCGCTAAC |
| | CAGTTTAATTCCGCAATTGGAAAGATCCAAGATTCACTCAG |
| | CTCAACCCCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGGATCCCCCAGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGTATATC |
| | AAATGGCCCTGGTACATTTGGCTGGGGTTTATCGCCGGACT |
| | GATTGCCATCGTCATGGTGACCATCATGCTGTGTTGCATGA |
| | CCTCCTGTTGTTCCTGTCTGAAGGGCTGCTGTAGTTGCGGCT |
| | CTTGCTGTAAATTCGACGAAGATGATAGCGAGCCCGTGCTG |
| | AAGGGCGTGAAGCTGCATTATACCTGA |
| SARS-CoV-2 S protein | (SEQ ID NO: 129) |
| mutated to replace | MFVFLVLLPLVSSQCVNLTTRTQLPPAYTNSFTRGVYYPDKVF |
| residues 817, 892, 899, | RSSVLHSTQDLFLPFFSNVTWFHAIHVSGTNGTKRFDNPVLPF |
| 942, 986 and 987 with | NDGVYFASTEKSNIIRGWIFGTTLDSKTQSLLIVNNATNVVIKV |
| proline | CEFQFCNDPFLGVYYHKNNKSWMESEFRVYSSANNCTFEYVS |
| | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPRRARSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSPIEDL |
| | LFNKVTLADAGFIKQYGDCLGDIAARDLICAQKFNGLTVLPPL |
| | LTDEMIAQYTSALLAGTITSGWTFGAGPALQIPFPMQMAYRF |
| | NGIGVTQNVLYENQKLIANQFNSAIGKIQDSLSSTPSALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 130) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove a | CACCAACTCCTTCACCAGAGGCGTGTATTACCCAGACAAGG |
| furin cleavage site and | TGTTTAGAAGCAGCGTGCTGCACTCTACCCAGGACCTCTTT |
| to replace residues 817, | CTGCCCTTTTTCAGCAACGTGACATGGTTTCACGCAATTCA |
| 892, 899, 942, 986 and | CGTGTCCGGCACTAATGGCACAAAGCGGTTCGACAATCCA |
| 987 with proline | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTGGCTCCGCCTCTTCCGTTGCTAGTCAGTCTATTATTGCC |
| | TATACCATGAGCCTCGGAGCTGAGAATAGCGTGGCCTACTC |
| | CAATAATTCCATCGCAATCCCTACTAACTTCACTATTTCTGT |
| | GACCACCGAGATCCTGCCTGTGTCTATGACTAAGACTAGCG |
| | TTGATTGTACCATGTATATTTGTGGCGACTCTACCGAATGTT |
| | CTAACCTGCTGCTTCAGTACGGCTCATTTTGCACACAGCTG |
| | AACAGAGCCCTGACTGGGATCGCTGTGGAGCAGGACAAGA |
| | ACACACAGGAGGTGTTTGCACAGGTGAAGCAGATCTATAA |
| | GACCCCTCCTATTAAGGATTTCGGCGGATTCAATTTCTCAC |
| | AGATTCTGCCAGACCCCAGTAAGCCTTCCAAGAGGAGCCCT |
| | ATCGAGGATCTCCTGTTTAACAAGGTGACCCTGGCAGACGC |
| | CGGCTTTATTAAGCAATATGGGGATTGCCTGGGCGACATTG |
| | CTGCCAGAGACCTGATTTGCGCCCAGAAATTCAATGGCCTC |
| | ACAGTGCTGCCACCTCTGCTGACCGACGAGATGATCGCTCA |
| | ATACACTAGCGCACTGCTGGCCGGAACCATCACATCAGGCT |
| | GGACCTTCGGGGCCGGACCAGCACTGCAGATTCCATTCCCT |
| | ATGCAGATGGCCTATAGATTCAACGGCATTGGCGTCACACA |
| | GAACGTGCTGTACGAAAACCAGAAGCTCATCGCTAACCAG |
| | TTTAATTCCGCAATTGGAAAGATCCAAGATTCACTCAGCTC |
| | AACCCCCTCTGCACTCGGAAAGCTGCAGGACGTGGTCAAC |
| | CAGAATGCTCAGGCCCTGAACACACTCGTCAAGCAGCTGTC |
| | CTCTAACTTTGGCGCTATCAGCTCCGTTCTGAACGACATTCT |
| | GAGCCGCCTGGATCCCCCAGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTGACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGTATATCAA |
| | ATGGCCCTGGTACATTTGGCTGGGGTTTATCGCCGGACTGA |
| | TTGCCATCGTCATGGTGACCATCATGCTGTGTTGCATGACC |
| | TCCTGTTGTTCCTGTCTGAAGGGCTGCTGTAGTTGCGGCTCT |
| | TGCTGTAAATTCGACGAAGATGATAGCGAGCCCGTGCTGA |
| | AGGGCGTGAAGCTGCATTATACCTGA |
| SARS-CoV-2 S protein | (SEQ ID NO: 131) |
| mutated to remove a | MFVFLVLLPLVSSQCVNLTTRTQLPPAYTNSFTRGVYYPDKVF |
| furin cleavage site and | RSSVLHSTQDLFLPFFSNVTWFHAIHVSGTNGTKRFDNPVLPF |
| to replace residues 817, | NDGVYFASTEKSNIIRGWIFGTTLDSKTQSLLIVNNATNVVIKV |
| 892, 899, 942, 986 and | CEFQFCNDPFLGVYYHKNNKSWMESEFRVYSSANNCTFEYVS |
| 987 with proline | QPFLMDLEGKQGNFKNLREFVFKNIDGYFKIYSKHTPINLVRD |
| | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSPIEDL |
| | LFNKVTLADAGFIKQYGDCLGDIAARDLICAQKFNGLTVLPPL |
| | LTDEMIAQYTSALLAGTITSGWTFGAGPALQIPFPMQMAYRF |
| | NGIGVTQNVLYENQKLIANQFNSAIGKIQDSLSSTPSALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 132) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to replace | CACCAACTCCTTCACCAGAGGCGTGTATTACCCAGACAAGG |
| residues 817, 892, 899, | TGTTTAGAAGCAGCGTGCTGCACTCTACCCAGGACCTCTTT |
| 942, 986 and 987 with | CTGCCCTTTTTCAGCAACGTGACATGGTTTCACGCAATTCA |
| proline and which | CGTGTCCGGCACTAATGGCACAAAGCGGTTCGACAATCCA |
| contains the D614G | GTCCTGCCTTTCAACGATGGCGTCTACTTTGCATCTACTGA |
| mutation | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGGCGTCAATTGCACAGAAGTGCCAGTTGCTATCCACGC |
| | AGACCAGCTGACTCCCACATGGCGGGTGTATAGCACCGGA |
| | TCCAACGTGTTTCAGACCCGCGCCGGATGTCTCATTGGGGC |
| | CGAGCACGTGAATAACAGCTACGAGTGCGACATCCCCATT |
| | GGCGCCGGCATTTGTGCGTCTTACCAGACTCAGACCAACTC |
| | TCCTAGAAGGGCCAGGTCCGTTGCTAGTCAGTCTATTATTG |
| | CCTATACCATGAGCCTCGGAGCTGAGAATAGCGTGGCCTAC |
| | TCCAATAATTCCATCGCAATCCCTACTAACTTCACTATTTCT |
| | GTGACCACCGAGATCCTGCCTGTGTCTATGACTAAGACTAG |
| | CGTTGATTGTACCATGTATATTTGTGGCGACTCTACCGAAT |
| | GTTCTAACCTGCTGCTTCAGTACGGCTCATTTTGCACACAG |
| | CTGAACAGAGCCCTGACTGGGATCGCTGTGGAGCAGGACA |
| | AGAACACACAGGAGGTGTTTGCACAGGTGAAGCAGATCTA |
| | TAAGACCCCTCCTATTAAGGATTTCGGCGGATTCAATTTCT |
| | CACAGATTCTGCCAGACCCCAGTAAGCCTTCCAAGAGGAG |
| | CCCTATCGAGGATCTCCTGTTTAACAAGGTGACCCTGGCAG |
| | ACGCCGGCTTTATTAAGCAATATGGGGATTGCCTGGGCGAC |
| | ATTGCTGCCAGAGACCTGATTTGCGCCCAGAAATTCAATGG |
| | CCTCACAGTGCTGCCACCTCTGCTGACCGACGAGATGATCG |
| | CTCAATACACTAGCGCACTGCTGGCCGGAACCATCACATCA |
| | GGCTGGACCTTCGGGGCCGGACCAGCACTGCAGATTCCATT |
| | CCCTATGCAGATGGCCTATAGATTCAACGGCATTGGCGTCA |
| | CACAGAACGTGCTGTACGAAAACCAGAAGCTCATCGCTAA |
| | CCAGTTTAATTCCGCAATTGGAAAGATCCAAGATTCACTCA |
| | GCTCAACCCCCTCTGCACTCGGAAAGCTGCAGGACGTGGTC |
| | AACCAGAATGCTCAGGCCCTGAACACACTCGTCAAGCAGC |
| | TGTCCTCTAACTTTGGCGCTATCAGCTCCGTTCTGAACGAC |
| | ATTCTGAGCCGCCTGGATCCCCCAGAGGCTGAAGTCCAGAT |
| | TGACCGCCTGATTACCGGCCGGCTGCAGTCTCTGCAAACAT |
| | ACGTGACCCAGCAGCTGATCAGAGCAGCCGAGATCCGGGC |
| | ATCCGCAAATCTGGCAGCAACTAAGATGAGCGAATGCGTG |
| | CTGGGCCAGTCCAAGCGGGTGGACTTTTGTGGCAAGGGCT |
| | ACCACCTGATGAGCTTCCCCCAGAGCGCCCCACATGGCGTT |
| | GTTTTTCTGCACGTGACCTATGTCCCTGCTCAGGAAAAGAA |
| | CTTTACAACTGCTCCTGCTATCTGCCATGACGGCAAGGCCC |
| | ACTTCCCACGGGAGGGAGTGTTTGTGTCCAATGGCACACAC |
| | TGGTTCGTGACCCAGAGGAACTTCTATGAACCCCAGATCAT |
| | CACCACTGACAATACCTTCGTGTCTGGAAATTGCGACGTCG |
| | TGATCGGCATCGTTAACAACACCGTGTACGACCCTCTCCAG |
| | CCAGAGCTGGACTCCTTTAAGGAGGAACTGGATAAGTATTT |
| | TAAGAACCACACAAGCCCAGATGTGGATCTCGGGGACATC |
| | TCCGGAATTAACGCCTCCGTGGTGAATATCCAGAAGGAGA |
| | TTGACCGCCTAAATGAAGTTGCCAAGAACCTCAATGAGTCT |
| | CTGATTGATCTGCAGGAACTGGGCAAGTATGAGCAGTATAT |
| | CAAATGGCCCTGGTACATTTGGCTGGGGTTTATCGCCGGAC |
| | TGATTGCCATCGTCATGGTGACCATCATGCTGTGTTGCATG |
| | ACCTCCTGTTGTTCCTGTCTGAAGGGCTGCTGTAGTTGCGG |
| | CTCTTGCTGTAAATTCGACGAAGATGATAGCGAGCCCGTGC |
| | TGAAGGGCGTGAAGCTGCATTATACCTGA |
| SARS-CoV-2 S protein | (SEQ ID NO: 133) |
| mutated to replace | MFVFLVLLPLVSSQCVNLTTRTQLPPAYTNSFTRGVYYPDKVF |
| residues 817, 892, 899, | RSSVLHSTQDLFLPFFSNVTWFHAIHVSGTNGTKRFDNPVLPF |
| 942, 986 and 987 with | NDGVYFASTEKSNIIRGWIFGTTLDSKTQSLLIVNNATNVVIKV |
| proline and which | CEFQFCNDPFLGVYYHKNNKSWMESEFRVYSSANNCTFEYVS |
| contains the D614G | QPFLMDLEGKQGNFKNLREFVFKNIDGYFKIYSKHTPINLVRD |
| mutation | LPQGFSALEPLVDLPIGINITRFQTLLALHRSYLTPGDSSSGWT |
| | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQGVNCTEVPVAIHADQLTPTWRVYSTGSNVFQ |
| | TRAGCLIGAEHVNNSYECDIPIGAGICASYQTQTNSPRRARSV |
| | ASQSIIAYTMSLGAENSVAYSNNSIAIPTNFTISVTTEILPVSMT |
| | KTSVDCTMYICGDSTECSNLLLQYGSFCTQLNRALTGIAVEQD |
| | KNTQEVFAQVKQIYKTPPIKDFGGFNFSQILPDPSKPSKRSPIED |
| | LLFNKVTLADAGFIKQYGDCLGDIAARDLICAQKENGLTVLPP |
| | LLTDEMIAQYTSALLAGTITSGWTFGAGPALQIPFPMQMAYR |
| | FNGIGVTQNVLYENQKLIANQFNSAIGKIQDSLSSTPSALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 134) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove a | CACCAACTCCTTCACCAGAGGCGTGTATTACCCAGACAAGG |
| furin cleavage site, to | TGTTTAGAAGCAGCGTGCTGCACTCTACCCAGGACCTCTTT |
| replace residues 817, | CTGCCCTTTTTCAGCAACGTGACATGGTTTCACGCAATTCA |
| 892, 899, 942, 986 and | CGTGTCCGGCACTAATGGCACAAAGCGGTTCGACAATCCA |
| 987 with proline and | GTCCTGCCTTTCAACGATGGCGTCTACTTTGCATCTACTGA |
| which contains the | GAAATCCAATATCATTAGGGGATGGATCTTCGGCACAACCC |
| D614G mutation | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGGCGTCAATTGCACAGAAGTGCCAGTTGCTATCCACGC |
| | AGACCAGCTGACTCCCACATGGCGGGTGTATAGCACCGGA |
| | TCCAACGTGTTTCAGACCCGCGCCGGATGTCTCATTGGGGC |
| | CGAGCACGTGAATAACAGCTACGAGTGCGACATCCCCATT |
| | GGCGCCGGCATTTGTGCGTCTTACCAGACTCAGACCAACTC |
| | TCCTGGCTCCGCCTCTTCCGTTGCTAGTCAGTCTATTATTGC |
| | CTATACCATGAGCCTCGGAGCTGAGAATAGCGTGGCCTACT |
| | CCAATAATTCCATCGCAATCCCTACTAACTTCACTATTTCTG |
| | TGACCACCGAGATCCTGCCTGTGTCTATGACTAAGACTAGC |
| | GTTGATTGTACCATGTATATTTGTGGCGACTCTACCGAATG |
| | TTCTAACCTGCTGCTTCAGTACGGCTCATTTTGCACACAGCT |
| | GAACAGAGCCCTGACTGGGATCGCTGTGGAGCAGGACAAG |
| | AACACACAGGAGGTGTTTGCACAGGTGAAGCAGATCTATA |
| | AGACCCCTCCTATTAAGGATTTCGGCGGATTCAATTTCTCA |
| | CAGATTCTGCCAGACCCCAGTAAGCCTTCCAAGAGGAGCC |
| | CTATCGAGGATCTCCTGTTTAACAAGGTGACCCTGGCAGAC |
| | GCCGGCTTTATTAAGCAATATGGGGATTGCCTGGGCGACAT |
| | TGCTGCCAGAGACCTGATTTGCGCCCAGAAATTCAATGGCC |
| | TCACAGTGCTGCCACCTCTGCTGACCGACGAGATGATCGCT |
| | CAATACACTAGCGCACTGCTGGCCGGAACCATCACATCAG |
| | GCTGGACCTTCGGGGCCGGACCAGCACTGCAGATTCCATTC |
| | CCTATGCAGATGGCCTATAGATTCAACGGCATTGGCGTCAC |
| | ACAGAACGTGCTGTACGAAAACCAGAAGCTCATCGCTAAC |
| | CAGTTTAATTCCGCAATTGGAAAGATCCAAGATTCACTCAG |
| | CTCAACCCCCTCTGCACTCGGAAAGCTGCAGGACGTGGTCA |
| | ACCAGAATGCTCAGGCCCTGAACACACTCGTCAAGCAGCT |
| | GTCCTCTAACTTTGGCGCTATCAGCTCCGTTCTGAACGACA |
| | TTCTGAGCCGCCTGGATCCCCCAGAGGCTGAAGTCCAGATT |
| | GACCGCCTGATTACCGGCCGGCTGCAGTCTCTGCAAACATA |
| | CGTGACCCAGCAGCTGATCAGAGCAGCCGAGATCCGGGCA |
| | TCCGCAAATCTGGCAGCAACTAAGATGAGCGAATGCGTGC |
| | TGGGCCAGTCCAAGCGGGTGGACTTTTGTGGCAAGGGCTA |
| | CCACCTGATGAGCTTCCCCCAGAGCGCCCCACATGGCGTTG |
| | TTTTTCTGCACGTGACCTATGTCCCTGCTCAGGAAAAGAAC |
| | TTTACAACTGCTCCTGCTATCTGCCATGACGGCAAGGCCCA |
| | CTTCCCACGGGAGGGAGTGTTTGTGTCCAATGGCACACACT |
| | GGTTCGTGACCCAGAGGAACTTCTATGAACCCCAGATCATC |
| | ACCACTGACAATACCTTCGTGTCTGGAAATTGCGACGTCGT |
| | GATCGGCATCGTTAACAACACCGTGTACGACCCTCTCCAGC |
| | CAGAGCTGGACTCCTTTAAGGAGGAACTGGATAAGTATTTT |
| | AAGAACCACACAAGCCCAGATGTGGATCTCGGGGACATCT |
| | CCGGAATTAACGCCTCCGTGGTGAATATCCAGAAGGAGAT |
| | TGACCGCCTAAATGAAGTTGCCAAGAACCTCAATGAGTCTC |
| | TGATTGATCTGCAGGAACTGGGCAAGTATGAGCAGTATATC |
| | AAATGGCCCTGGTACATTTGGCTGGGGTTTATCGCCGGACT |
| | GATTGCCATCGTCATGGTGACCATCATGCTGTGTTGCATGA |
| | CCTCCTGTTGTTCCTGTCTGAAGGGCTGCTGTAGTTGCGGCT |
| | CTTGCTGTAAATTCGACGAAGATGATAGCGAGCCCGTGCTG |
| | AAGGGCGTGAAGCTGCATTATACCTGA |
| SARS-CoV-2 S protein | (SEQ ID NO: 135) |
| mutated to remove a | MFVFLVLLPLVSSQCVNLTTRTQLPPAYTNSFTRGVYYPDKVF |
| furin cleavage site, to | RSSVLHSTQDLFLPFFSNVTWFHAIHVSGTNGTKRFDNPVLPF |
| replace residues 817, | NDGVYFASTEKSNIIRGWIFGTTLDSKTQSLLIVNNATNVVIKV |
| 892, 899, 942, 986 and | CEFQFCNDPFLGVYYHKNNKSWMESEFRVYSSANNCTFEYVS |
| 987 with proline and | QPFLMDLEGKQGNFKNLREFVFKNIDGYFKIYSKHTPINLVRD |
| which contains the | LPQGFSALEPLVDLPIGINITRFQTLLALHRSYLTPGDSSSGWT |
| D614G mutation | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQGVNCTEVPVAIHADQLTPTWRVYSTGSNVFQ |
| | TRAGCLIGAEHVNNSYECDIPIGAGICASYQTQTNSPGSASSVA |
| | SQSIIAYTMSLGAENSVAYSNNSIAIPTNFTISVTTEILPVSMTK |
| | TSVDCTMYICGDSTECSNLLLQYGSFCTQLNRALTGIAVEQDK |
| | NTQEVFAQVKQIYKTPPIKDFGGFNFSQILPDPSKPSKRSPIEDL |
| | LFNKVTLADAGFIKQYGDCLGDIAARDLICAQKFNGLTVLPPL |
| | LTDEMIAQYTSALLAGTITSGWTFGAGPALQIPFPMQMAYRF |
| | NGIGVTQNVLYENQKLIANQFNSAIGKIQDSLSSTPSALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVKLHYT |
| Optimized nucleotide | (SEQ ID NO: 136) |
| sequence encoding a | ATGTTCCTGCTGACAACAAAAAGAACCATGTTTGTGTTCCT |
| SARS-CoV-2 S protein | GGTGCTGCTGCCTCTGGTGTCCTCACAGTGTGTCAACCTGA |
| mutated to remove a | CAACAAGAACTCAGCTGCCACCAGCCTACACCAACTCCTTC |
| furin cleavage site, to | ACCAGAGGCGTGTATTACCCAGACAAGGTGTTTAGAAGCA |
| replace residues 817, | GCGTGCTGCACTCTACCCAGGACCTCTTTCTGCCCTTTTTCA |
| 892, 899, 942, 986 and | GCAACGTGACATGGTTTCACGCAATTCACGTGTCCGGCACT |
| 987 with proline and | AATGGCACAAAGCGGTTCGACAATCCAGTCCTGCCTTTCAA |
| containing an extended | CGATGGCGTCTACTTTGCATCTACTGAGAAATCCAATATCA |
| signal sequence | TTAGGGGATGGATCTTCGGCACAACCCTGGATTCTAAGACC |
| | CAGAGCCTGCTGATCGTCAACAACGCCACAAACGTGGTCA |
| | TTAAGGTTTGCGAGTTTCAGTTCTGTAACGATCCTTTTCTGG |
| | GCGTGTATTATCATAAGAACAATAAGAGCTGGATGGAGTC |
| | CGAGTTTAGAGTGTATAGCTCTGCAAATAATTGTACCTTTG |
| | AGTACGTGAGCCAGCCCTTTCTGATGGACCTGGAGGGAAA |
| | ACAAGGAAACTTCAAAAACCTGCGGGAATTCGTTTTCAAA |
| | AACATCGACGGCTATTTCAAGATCTATAGCAAGCATACCCC |
| | AATCAACCTCGTGAGGGACCTCCCCCAGGGCTTTAGCGCAC |
| | TGGAGCCACTGGTTGACCTGCCTATCGGCATTAATATCACA |
| | AGATTTCAGACCCTGCTGGCACTGCATAGAAGCTATCTGAC |
| | CCCTGGAGACTCCTCTAGTGGGTGGACTGCCGGCGCCGCTG |
| | CCTACTATGTGGGCTATCTGCAGCCACGGACATTCCTGCTG |
| | AAATACAATGAGAACGGGACAATCACAGATGCTGTTGATT |
| | GCGCACTCGACCCCCTGTCCGAGACAAAGTGCACTCTCAAG |
| | AGCTTTACCGTCGAGAAGGGCATCTATCAGACCTCAAACTT |
| | CAGGGTGCAGCCCACAGAATCTATCGTGCGCTTCCCTAATA |
| | TCACTAACCTGTGTCCTTTCGGTGAAGTGTTCAACGCCACC |
| | AGGTTTGCTAGCGTGTATGCCTGGAACAGGAAGAGGATCT |
| | CTAACTGCGTCGCCGACTATTCCGTGCTGTATAACAGCGCC |
| | TCCTTCTCCACATTCAAATGCTATGGAGTGAGCCCGACAAA |
| | ACTGAACGATCTCTGCTTTACAAATGTCTACGCCGACTCTT |
| | TTGTGATCAGAGGGGACGAGGTCCGGCAGATCGCACCAGG |
| | ACAGACAGGCAAGATTGCTGACTACAACTATAAGCTGCCT |
| | GACGACTTCACAGGATGTGTGATCGCATGGAACTCAAACA |
| | ATCTGGACTCCAAAGTCGGGGGCAACTATAATTACCTGTAT |
| | CGCCTGTTCCGGAAGTCCAACCTGAAGCCCTTCGAGAGGG |
| | ACATCAGTACAGAGATCTATCAGGCTGGCTCCACCCCTTGC |
| | AATGGCGTCGAAGGCTTTAATTGTTATTTTCCCCTGCAGTCT |
| | TACGGGTTTCAGCCTACTAATGGAGTTGGGTACCAGCCATA |
| | CAGAGTGGTCGTGCTCAGCTTCGAGCTCCTGCATGCTCCAG |
| | CTACAGTTTGCGGGCCAAAGAAGTCCACTAACCTGGTGAA |
| | GAATAAGTGCGTCAACTTCAACTTTAACGGGCTCACCGGCA |
| | CCGGCGTGCTGACTGAGAGCAACAAGAAGTTTCTGCCATTT |
| | CAACAGTTTGGACGGGACATTGCCGACACCACCGATGCCG |
| | TTCGGGATCCACAGACCCTGGAAATTCTGGACATTACACCG |
| | TGCAGCTTCGGGGGCGTGAGCGTGATCACACCCGGAACCA |
| | ATACAAGCAACCAGGTTGCCGTCCTGTATCAGGATGTCAAT |
| | TGCACAGAAGTGCCAGTTGCTATCCACGCAGACCAGCTGA |
| | CTCCCACATGGCGGGTGTATAGCACCGGATCCAACGTGTTT |
| | CAGACCCGCGCCGGATGTCTCATTGGGGCCGAGCACGTGA |
| | ATAACAGCTACGAGTGCGACATCCCCATTGGCGCCGGCATT |
| | TGTGCGTCTTACCAGACTCAGACCAACTCTCCTGGCTCCGC |
| | CTCTTCCGTTGCTAGTCAGTCTATTATTGCCTATACCATGAG |
| | CCTCGGAGCTGAGAATAGCGTGGCCTACTCCAATAATTCCA |
| | TCGCAATCCCTACTAACTTCACTATTTCTGTGACCACCGAG |
| | ATCCTGCCTGTGTCTATGACTAAGACTAGCGTTGATTGTAC |
| | CATGTATATTTGTGGCGACTCTACCGAATGTTCTAACCTGC |
| | TGCTTCAGTACGGCTCATTTTGCACACAGCTGAACAGAGCC |
| | CTGACTGGGATCGCTGTGGAGCAGGACAAGAACACACAGG |
| | AGGTGTTTGCACAGGTGAAGCAGATCTATAAGACCCCTCCT |
| | ATTAAGGATTTCGGCGGATTCAATTTCTCACAGATTCTGCC |
| | AGACCCCAGTAAGCCTTCCAAGAGGAGCCCTATCGAGGAT |
| | CTCCTGTTTAACAAGGTGACCCTGGCAGACGCCGGCTTTAT |
| | TAAGCAATATGGGGATTGCCTGGGCGACATTGCTGCCAGA |
| | GACCTGATTTGCGCCCAGAAATTCAATGGCCTCACAGTGCT |
| | GCCACCTCTGCTGACCGACGAGATGATCGCTCAATACACTA |
| | GCGCACTGCTGGCCGGAACCATCACATCAGGCTGGACCTTC |
| | GGGGCCGGACCAGCACTGCAGATTCCATTCCCTATGCAGAT |
| | GGCCTATAGATTCAACGGCATTGGCGTCACACAGAACGTG |
| | CTGTACGAAAACCAGAAGCTCATCGCTAACCAGTTTAATTC |
| | CGCAATTGGAAAGATCCAAGATTCACTCAGCTCAACCCCCT |
| | CTGCACTCGGAAAGCTGCAGGACGTGGTCAACCAGAATGC |
| | TCAGGCCCTGAACACACTCGTCAAGCAGCTGTCCTCTAACT |
| | TTGGCGCTATCAGCTCCGTTCTGAACGACATTCTGAGCCGC |
| | CTGGATCCCCCAGAGGCTGAAGTCCAGATTGACCGCCTGAT |
| | TACCGGCCGGCTGCAGTCTCTGCAAACATACGTGACCCAGC |
| | AGCTGATCAGAGCAGCCGAGATCCGGGCATCCGCAAATCT |
| | GGCAGCAACTAAGATGAGCGAATGCGTGCTGGGCCAGTCC |
| | AAGCGGGTGGACTTTTGTGGCAAGGGCTACCACCTGATGA |
| | GCTTCCCCCAGAGCGCCCCACATGGCGTTGTTTTTCTGCAC |
| | GTGACCTATGTCCCTGCTCAGGAAAAGAACTTTACAACTGC |
| | TCCTGCTATCTGCCATGACGGCAAGGCCCACTTCCCACGGG |
| | AGGGAGTGTTTGTGTCCAATGGCACACACTGGTTCGTGACC |
| | CAGAGGAACTTCTATGAACCCCAGATCATCACCACTGACA |
| | ATACCTTCGTGTCTGGAAATTGCGACGTCGTGATCGGCATC |
| | GTTAACAACACCGTGTACGACCCTCTCCAGCCAGAGCTGGA |
| | CTCCTTTAAGGAGGAACTGGATAAGTATTTTAAGAACCACA |
| | CAAGCCCAGATGTGGATCTCGGGGACATCTCCGGAATTAA |
| | CGCCTCCGTGGTGAATATCCAGAAGGAGATTGACCGCCTA |
| | AATGAAGTTGCCAAGAACCTCAATGAGTCTCTGATTGATCT |
| | GCAGGAACTGGGCAAGTATGAGCAGTATATCAAATGGCCC |
| | TGGTACATTTGGCTGGGGTTTATCGCCGGACTGATTGCCAT |
| | CGTCATGGTGACCATCATGCTGTGTTGCATGACCTCCTGTT |
| | GTTCCTGTCTGAAGGGCTGCTGTAGTTGCGGCTCTTGCTGT |
| | AAATTCGACGAAGATGATAGCGAGCCCGTGCTGAAGGGCG |
| | TGAAGCTGCATTATACCTGA |
| SARS-CoV-2 S protein | (SEQ ID NO: 137) |
| mutated to remove a | MFLLTTKRTMFVFLVLLPLVSSQCVNLTTRTQLPPAYTNSFTR |
| furin cleavage site, to | GVYYPDKVFRSSVLHSTQDLFLPFFSNVTWFHAIHVSGTNGTK |
| replace residues 817, | RFDNPVLPFNDGVYFASTEKSNIIRGWIFGTTLDSKTQSLLIVN |
| 892, 899, 942, 986 and | NATNVVIKVCEFQFCNDPFLGVYYHKNNKSWMESEFRVYSS |
| 987 with proline and | ANNCTFEYVSQPFLMDLEGKQGNFKNLREFVFKNIDGYFKIYS |
| containing an extended | KHTPINLVRDLPQGFSALEPLVDLPIGINITRFQTLLALHRSYLT |
| signal sequence | PGDSSSGWTAGAAAYYVGYLQPRTFLLKYNENGTITDAVDC |
| | ALDPLSETKCTLKSFTVEKGIYQTSNFRVQPTESIVRFPNITNLC |
| | PFGEVFNATRFASVYAWNRKRISNCVADYSVLYNSASFSTFK |
| | CYGVSPTKLNDLCFTNVYADSFVIRGDEVRQIAPGQTGKIADY |
| | NYKLPDDFTGCVIAWNSNNLDSKVGGNYNYLYRLFRKSNLK |
| | PFERDISTEIYQAGSTPCNGVEGFNCYFPLQSYGFQPTNGVGY |
| | QPYRVVVLSFELLHAPATVCGPKKSTNLVKNKCVNFNFNGLT |
| | GTGVLTESNKKFLPFQQFGRDIADTTDAVRDPQTLEILDITPCS |
| | FGGVSVITPGTNTSNQVAVLYQDVNCTEVPVAIHADQLTPTW |
| | RVYSTGSNVFQTRAGCLIGAEHVNNSYECDIPIGAGICASYQT |
| | QTNSPGSASSVASQSIIAYTMSLGAENSVAYSNNSIAIPTNFTIS |
| | VTTEILPVSMTKTSVDCTMYICGDSTECSNLLLQYGSFCTQLN |
| | RALTGIAVEQDKNTQEVFAQVKQIYKTPPIKDFGGFNFSQILPD |
| | PSKPSKRSPIEDLLFNKVTLADAGFIKQYGDCLGDIAARDLICA |
| | QKFNGLTVLPPLLTDEMIAQYTSALLAGTITSGWTFGAGPALQ |
| | IPFPMQMAYRFNGIGVTQNVLYENQKLIANQFNSAIGKIQDSL |
| | SSTPSALGKLQDVVNQNAQALNTLVKQLSSNFGAISSVLNDIL |
| | SRLDPPEAEVQIDRLITGRLQSLQTYVTQQLIRAAEIRASANLA |
| | ATKMSECVLGQSKRVDFCGKGYHLMSFPQSAPHGVVFLHVT |
| | YVPAQEKNFTTAPAICHDGKAHFPREGVFVSNGTHWFVTQRN |
| | FYEPQIITTDNTFVSGNCDVVIGIVNNTVYDPLQPELDSFKEEL |
| | DKYFKNHTSPDVDLGDISGINASVVNIQKEIDRLNEVAKNLNE |
| | SLIDLQELGKYEQYIKWPWYIWLGFIAGLIAIVMVTIMLCCMT |
| | SCCSCLKGCCSCGSCCKFDEDDSEPVLKGVKLHYT |
| Optimized nucleotide | (SEQ ID NO: 138) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove a | CACCAACTCCTTCACCAGAGGCGTGTATTACCCAGACAAGG |
| furin cleavage site, to | TGTTTAGAAGCAGCGTGCTGCACTCTACCCAGGACCTCTTT |
| replace residues 817, | CTGCCCTTTTTCAGCAACGTGACATGGTTTCACGCAATTCA |
| 892, 899, 942, 986 and | CGTGTCCGGCACTAATGGCACAAAGCGGTTCGACAATCCA |
| 987 with proline and to | GTCCTGCCTTTCAACGATGGCGTCTACTTTGCATCTACTGA |
| mutate the ER retrieval | GAAATCCAATATCATTAGGGGATGGATCTTCGGCACAACCC |
| signal | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGACCTCCCCCAG |
| | GGCTTTAGCGCACTGGAGCCACTGGTTGACCTGCCTATCGG |
| | CATTAATATCACAAGATTTCAGACCCTGCTGGCACTGCATA |
| | GAAGCTATCTGACCCCTGGAGACTCCTCTAGTGGGTGGACT |
| | GCCGGCGCCGCTGCCTACTATGTGGGCTATCTGCAGCCACG |
| | GACATTCCTGCTGAAATACAATGAGAACGGGACAATCACA |
| | GATGCTGTTGATTGCGCACTCGACCCCCTGTCCGAGACAAA |
| | GTGCACTCTCAAGAGCTTTACCGTCGAGAAGGGCATCTATC |
| | AGACCTCAAACTTCAGGGTGCAGCCCACAGAATCTATCGTG |
| | CGCTTCCCTAATATCACTAACCTGTGTCCTTTCGGTGAAGT |
| | GTTCAACGCCACCAGGTTTGCTAGCGTGTATGCCTGGAACA |
| | GGAAGAGGATCTCTAACTGCGTCGCCGACTATTCCGTGCTG |
| | TATAACAGCGCCTCCTTCTCCACATTCAAATGCTATGGAGT |
| | GAGCCCGACAAAACTGAACGATCTCTGCTTTACAAATGTCT |
| | ACGCCGACTCTTTTGTGATCAGAGGGGACGAGGTCCGGCA |
| | GATCGCACCAGGACAGACAGGCAAGATTGCTGACTACAAC |
| | TATAAGCTGCCTGACGACTTCACAGGATGTGTGATCGCATG |
| | GAACTCAAACAATCTGGACTCCAAAGTCGGGGGCAACTAT |
| | AATTACCTGTATCGCCTGTTCCGGAAGTCCAACCTGAAGCC |
| | CTTCGAGAGGGACATCAGTACAGAGATCTATCAGGCTGGC |
| | TCCACCCCTTGCAATGGCGTCGAAGGCTTTAATTGTTATTTT |
| | CCCCTGCAGTCTTACGGGTTTCAGCCTACTAATGGAGTTGG |
| | GTACCAGCCATACAGAGTGGTCGTGCTCAGCTTCGAGCTCC |
| | TGCATGCTCCAGCTACAGTTTGCGGGCCAAAGAAGTCCACT |
| | AACCTGGTGAAGAATAAGTGCGTCAACTTCAACTTTAACGG |
| | GCTCACCGGCACCGGCGTGCTGACTGAGAGCAACAAGAAG |
| | TTTCTGCCATTTCAACAGTTTGGACGGGACATTGCCGACAC |
| | CACCGATGCCGTTCGGGATCCACAGACCCTGGAAATTCTGG |
| | ACATTACACCGTGCAGCTTCGGGGGCGTGAGCGTGATCAC |
| | ACCCGGAACCAATACAAGCAACCAGGTTGCCGTCCTGTATC |
| | AGGATGTCAATTGCACAGAAGTGCCAGTTGCTATCCACGCA |
| | GACCAGCTGACTCCCACATGGCGGGTGTATAGCACCGGAT |
| | CCAACGTGTTTCAGACCCGCGCCGGATGTCTCATTGGGGCC |
| | GAGCACGTGAATAACAGCTACGAGTGCGACATCCCCATTG |
| | GCGCCGGCATTTGTGCGTCTTACCAGACTCAGACCAACTCT |
| | CCTGGCTCCGCCTCTTCCGTTGCTAGTCAGTCTATTATTGCC |
| | TATACCATGAGCCTCGGAGCTGAGAATAGCGTGGCCTACTC |
| | CAATAATTCCATCGCAATCCCTACTAACTTCACTATTTCTGT |
| | GACCACCGAGATCCTGCCTGTGTCTATGACTAAGACTAGCG |
| | TTGATTGTACCATGTATATTTGTGGCGACTCTACCGAATGTT |
| | CTAACCTGCTGCTTCAGTACGGCTCATTTTGCACACAGCTG |
| | AACAGAGCCCTGACTGGGATCGCTGTGGAGCAGGACAAGA |
| | ACACACAGGAGGTGTTTGCACAGGTGAAGCAGATCTATAA |
| | GACCCCTCCTATTAAGGATTTCGGCGGATTCAATTTCTCAC |
| | AGATTCTGCCAGACCCCAGTAAGCCTTCCAAGAGGAGCCCT |
| | ATCGAGGATCTCCTGTTTAACAAGGTGACCCTGGCAGACGC |
| | CGGCTTTATTAAGCAATATGGGGATTGCCTGGGCGACATTG |
| | CTGCCAGAGACCTGATTTGCGCCCAGAAATTCAATGGCCTC |
| | ACAGTGCTGCCACCTCTGCTGACCGACGAGATGATCGCTCA |
| | ATACACTAGCGCACTGCTGGCCGGAACCATCACATCAGGCT |
| | GGACCTTCGGGGCCGGACCAGCACTGCAGATTCCATTCCCT |
| | ATGCAGATGGCCTATAGATTCAACGGCATTGGCGTCACACA |
| | GAACGTGCTGTACGAAAACCAGAAGCTCATCGCTAACCAG |
| | TTTAATTCCGCAATTGGAAAGATCCAAGATTCACTCAGCTC |
| | AACCCCCTCTGCACTCGGAAAGCTGCAGGACGTGGTCAAC |
| | CAGAATGCTCAGGCCCTGAACACACTCGTCAAGCAGCTGTC |
| | CTCTAACTTTGGCGCTATCAGCTCCGTTCTGAACGACATTCT |
| | GAGCCGCCTGGATCCCCCAGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTGACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGTATATCAA |
| | ATGGCCCTGGTACATTTGGCTGGGGTTTATCGCCGGACTGA |
| | TTGCCATCGTCATGGTGACCATCATGCTGTGTTGCATGACC |
| | TCCTGTTGTTCCTGTCTGAAGGGCTGCTGTAGTTGCGGCTCT |
| | TGCTGTAAATTCGACGAAGATGATAGCGAGCCCGTGCTGA |
| | AGGGCGTGGCCCTGGCTTATACCTGA |
| SARS-CoV-2 S protein | (SEQ ID NO: 139) |
| mutated to remove a | MFVFLVLLPLVSSQCVNLTTRTQLPPAYTNSFTRGVYYPDKVF |
| furin cleavage site, to | RSSVLHSTQDLFLPFFSNVTWFHAIHVSGTNGTKRFDNPVLPF |
| replace residues 817, | NDGVYFASTEKSNIIRGWIFGTTLDSKTQSLLIVNNATNVVIKV |
| 892, 899, 942, 986 and | CEFQFCNDPFLGVYYHKNNKSWMESEFRVYSSANNCTFEYVS |
| 987 with proline and to | QPFLMDLEGKQGNFKNLREFVFKNIDGYFKIYSKHTPINLVRD |
| mutate the ER retrieval | LPQGFSALEPLVDLPIGINITRFQTLLALHRSYLTPGDSSSGWT |
| signal | AGAAAYYVGYLQPRTFLLKYNENGTITDAVDCALDPLSETKC |
| | TLKSFTVEKGIYQTSNFRVQPTESIVRFPNITNLCPFGEVFNATR |
| | FASVYAWNRKRISNCVADYSVLYNSASFSTFKCYGVSPTKLN |
| | DLCFTNVYADSFVIRGDEVRQIAPGQTGKIADYNYKLPDDFTG |
| | CVIAWNSNNLDSKVGGNYNYLYRLFRKSNLKPFERDISTEIYQ |
| | AGSTPCNGVEGFNCYFPLQSYGFQPTNGVGYQPYRVVVLSFE |
| | LLHAPATVCGPKKSTNLVKNKCVNFNFNGLTGTGVLTESNKK |
| | FLPFQQFGRDIADTTDAVRDPQTLEILDITPCSFGGVSVITPGTN |
| | TSNQVAVLYQDVNCTEVPVAIHADQLTPTWRVYSTGSNVFQT |
| | RAGCLIGAEHVNNSYECDIPIGAGICASYQTQTNSPGSASSVAS |
| | QSIIAYTMSLGAENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSPIEDL |
| | LFNKVTLADAGFIKQYGDCLGDIAARDLICAQKFNGLTVLPPL |
| | LTDEMIAQYTSALLAGTITSGWTFGAGPALQIPFPMQMAYRF |
| | NGIGVTQNVLYENQKLIANQFNSAIGKIQDSLSSTPSALGKLQ |
| | DVVNQNAQALNTLVKQLSSNFGAISSVLNDILSRLDPPEAEVQ |
| | IDRLITGRLQSLQTYVTQQLIRAAEIRASANLAATKMSECVLG |
| | QSKRVDFCGKGYHLMSFPQSAPHGVVFLHVTYVPAQEKNFTT |
| | APAICHDGKAHFPREGVFVSNGTHWFVTQRNFYEPQIITTDNT |
| | FVSGNCDVVIGIVNNTVYDPLQPELDSFKEELDKYFKNHTSPD |
| | VDLGDISGINASVVNIQKEIDRLNEVAKNLNESLIDLQELGKYE |
| | QYIKWPWYIWLGFIAGLIAIVMVTIMLCCMTSCCSCLKGCCSC |
| | GSCCKFDEDDSEPVLKGVALAYT |
| Optimized nucleotide | (SEQ ID NO: 140) |
| sequence encoding a | ATGTTCCTGCTGACAACAAAAAGAACCATGTTTGTGTTCCT |
| SARS-CoV-2 S protein | GGTGCTGCTGCCTCTGGTGTCCTCACAGTGTGTCAACCTGA |
| mutated to remove a | CAACAAGAACTCAGCTGCCACCAGCCTACACCAACTCCTTC |
| furin cleavage site, to | ACCAGAGGCGTGTATTACCCAGACAAGGTGTTTAGAAGCA |
| replace residues 817, | GCGTGCTGCACTCTACCCAGGACCTCTTTCTGCCCTTTTTCA |
| 892, 899, 942, 986 and | GCAACGTGACATGGTTTCACGCAATTCACGTGTCCGGCACT |
| 987 with proline, to | AATGGCACAAAGCGGTTCGACAATCCAGTCCTGCCTTTCAA |
| mutate the ER retrieval | CGATGGCGTCTACTTTGCATCTACTGAGAAATCCAATATCA |
| signal and containing an | TTAGGGGATGGATCTTCGGCACAACCCTGGATTCTAAGACC |
| extended signal | CAGAGCCTGCTGATCGTCAACAACGCCACAAACGTGGTCA |
| sequence | TTAAGGTTTGCGAGTTTCAGTTCTGTAACGATCCTTTTCTGG |
| | GCGTGTATTATCATAAGAACAATAAGAGCTGGATGGAGTC |
| | CGAGTTTAGAGTGTATAGCTCTGCAAATAATTGTACCTTTG |
| | AGTACGTGAGCCAGCCCTTTCTGATGGACCTGGAGGGAAA |
| | ACAAGGAAACTTCAAAAACCTGCGGGAATTCGTTTTCAAA |
| | AACATCGACGGCTATTTCAAGATCTATAGCAAGCATACCCC |
| | AATCAACCTCGTGAGGGACCTCCCCCAGGGCTTTAGCGCAC |
| | TGGAGCCACTGGTTGACCTGCCTATCGGCATTAATATCACA |
| | AGATTTCAGACCCTGCTGGCACTGCATAGAAGCTATCTGAC |
| | CCCTGGAGACTCCTCTAGTGGGTGGACTGCCGGCGCCGCTG |
| | CCTACTATGTGGGCTATCTGCAGCCACGGACATTCCTGCTG |
| | AAATACAATGAGAACGGGACAATCACAGATGCTGTTGATT |
| | GCGCACTCGACCCCCTGTCCGAGACAAAGTGCACTCTCAAG |
| | AGCTTTACCGTCGAGAAGGGCATCTATCAGACCTCAAACTT |
| | CAGGGTGCAGCCCACAGAATCTATCGTGCGCTTCCCTAATA |
| | TCACTAACCTGTGTCCTTTCGGTGAAGTGTTCAACGCCACC |
| | AGGTTTGCTAGCGTGTATGCCTGGAACAGGAAGAGGATCT |
| | CTAACTGCGTCGCCGACTATTCCGTGCTGTATAACAGCGCC |
| | TCCTTCTCCACATTCAAATGCTATGGAGTGAGCCCGACAAA |
| | ACTGAACGATCTCTGCTTTACAAATGTCTACGCCGACTCTT |
| | TTGTGATCAGAGGGGACGAGGTCCGGCAGATCGCACCAGG |
| | ACAGACAGGCAAGATTGCTGACTACAACTATAAGCTGCCT |
| | GACGACTTCACAGGATGTGTGATCGCATGGAACTCAAACA |
| | ATCTGGACTCCAAAGTCGGGGGCAACTATAATTACCTGTAT |
| | CGCCTGTTCCGGAAGTCCAACCTGAAGCCCTTCGAGAGGG |
| | ACATCAGTACAGAGATCTATCAGGCTGGCTCCACCCCTTGC |
| | AATGGCGTCGAAGGCTTTAATTGTTATTTTCCCCTGCAGTCT |
| | TACGGGTTTCAGCCTACTAATGGAGTTGGGTACCAGCCATA |
| | CAGAGTGGTCGTGCTCAGCTTCGAGCTCCTGCATGCTCCAG |
| | CTACAGTTTGCGGGCCAAAGAAGTCCACTAACCTGGTGAA |
| | GAATAAGTGCGTCAACTTCAACTTTAACGGGCTCACCGGCA |
| | CCGGCGTGCTGACTGAGAGCAACAAGAAGTTTCTGCCATTT |
| | CAACAGTTTGGACGGGACATTGCCGACACCACCGATGCCG |
| | TTCGGGATCCACAGACCCTGGAAATTCTGGACATTACACCG |
| | TGCAGCTTCGGGGGCGTGAGCGTGATCACACCCGGAACCA |
| | ATACAAGCAACCAGGTTGCCGTCCTGTATCAGGATGTCAAT |
| | TGCACAGAAGTGCCAGTTGCTATCCACGCAGACCAGCTGA |
| | CTCCCACATGGCGGGTGTATAGCACCGGATCCAACGTGTTT |
| | CAGACCCGCGCCGGATGTCTCATTGGGGCCGAGCACGTGA |
| | ATAACAGCTACGAGTGCGACATCCCCATTGGCGCCGGCATT |
| | TGTGCGTCTTACCAGACTCAGACCAACTCTCCTGGCTCCGC |
| | CTCTTCCGTTGCTAGTCAGTCTATTATTGCCTATACCATGAG |
| | CCTCGGAGCTGAGAATAGCGTGGCCTACTCCAATAATTCCA |
| | TCGCAATCCCTACTAACTTCACTATTTCTGTGACCACCGAG |
| | ATCCTGCCTGTGTCTATGACTAAGACTAGCGTTGATTGTAC |
| | CATGTATATTTGTGGCGACTCTACCGAATGTTCTAACCTGC |
| | TGCTTCAGTACGGCTCATTTTGCACACAGCTGAACAGAGCC |
| | CTGACTGGGATCGCTGTGGAGCAGGACAAGAACACACAGG |
| | AGGTGTTTGCACAGGTGAAGCAGATCTATAAGACCCCTCCT |
| | ATTAAGGATTTCGGCGGATTCAATTTCTCACAGATTCTGCC |
| | AGACCCCAGTAAGCCTTCCAAGAGGAGCCCTATCGAGGAT |
| | CTCCTGTTTAACAAGGTGACCCTGGCAGACGCCGGCTTTAT |
| | TAAGCAATATGGGGATTGCCTGGGCGACATTGCTGCCAGA |
| | GACCTGATTTGCGCCCAGAAATTCAATGGCCTCACAGTGCT |
| | GCCACCTCTGCTGACCGACGAGATGATCGCTCAATACACTA |
| | GCGCACTGCTGGCCGGAACCATCACATCAGGCTGGACCTTC |
| | GGGGCCGGACCAGCACTGCAGATTCCATTCCCTATGCAGAT |
| | GGCCTATAGATTCAACGGCATTGGCGTCACACAGAACGTG |
| | CTGTACGAAAACCAGAAGCTCATCGCTAACCAGTTTAATTC |
| | CGCAATTGGAAAGATCCAAGATTCACTCAGCTCAACCCCCT |
| | CTGCACTCGGAAAGCTGCAGGACGTGGTCAACCAGAATGC |
| | TCAGGCCCTGAACACACTCGTCAAGCAGCTGTCCTCTAACT |
| | TTGGCGCTATCAGCTCCGTTCTGAACGACATTCTGAGCCGC |
| | CTGGATCCCCCAGAGGCTGAAGTCCAGATTGACCGCCTGAT |
| | TACCGGCCGGCTGCAGTCTCTGCAAACATACGTGACCCAGC |
| | AGCTGATCAGAGCAGCCGAGATCCGGGCATCCGCAAATCT |
| | GGCAGCAACTAAGATGAGCGAATGCGTGCTGGGCCAGTCC |
| | AAGCGGGTGGACTTTTGTGGCAAGGGCTACCACCTGATGA |
| | GCTTCCCCCAGAGCGCCCCACATGGCGTTGTTTTTCTGCAC |
| | GTGACCTATGTCCCTGCTCAGGAAAAGAACTTTACAACTGC |
| | TCCTGCTATCTGCCATGACGGCAAGGCCCACTTCCCACGGG |
| | AGGGAGTGTTTGTGTCCAATGGCACACACTGGTTCGTGACC |
| | CAGAGGAACTTCTATGAACCCCAGATCATCACCACTGACA |
| | ATACCTTCGTGTCTGGAAATTGCGACGTCGTGATCGGCATC |
| | GTTAACAACACCGTGTACGACCCTCTCCAGCCAGAGCTGGA |
| | CTCCTTTAAGGAGGAACTGGATAAGTATTTTAAGAACCACA |
| | CAAGCCCAGATGTGGATCTCGGGGACATCTCCGGAATTAA |
| | CGCCTCCGTGGTGAATATCCAGAAGGAGATTGACCGCCTA |
| | AATGAAGTTGCCAAGAACCTCAATGAGTCTCTGATTGATCT |
| | GCAGGAACTGGGCAAGTATGAGCAGTATATCAAATGGCCC |
| | TGGTACATTTGGCTGGGGTTTATCGCCGGACTGATTGCCAT |
| | CGTCATGGTGACCATCATGCTGTGTTGCATGACCTCCTGTT |
| | GTTCCTGTCTGAAGGGCTGCTGTAGTTGCGGCTCTTGCTGT |
| | AAATTCGACGAAGATGATAGCGAGCCCGTGCTGAAGGGCG |
| | TGGCCCTGGCTTATACCTGA |
| SARS-CoV-2 S protein | (SEQ ID NO: 141) |
| mutated to remove a | MFLLTTKRTMFVFLVLLPLVSSQCVNLTTRTQLPPAYTNSFTR |
| furin cleavage site, to | GVYYPDKVFRSSVLHSTQDLFLPFFSNVTWFHAIHVSGTNGTK |
| replace residues 817, | RFDNPVLPFNDGVYFASTEKSNIIRGWIFGTTLDSKTQSLLIVN |
| 892, 899, 942, 986 and | NATNVVIKVCEFQFCNDPFLGVYYHKNNKSWMESEFRVYSS |
| 987 with proline, to | ANNCTFEYVSQPFLMDLEGKQGNFKNLREFVFKNIDGYFKIYS |
| mutate the ER retrieval | KHTPINLVRDLPQGFSALEPLVDLPIGINITRFQTLLALHRSYLT |
| signal and containing an | PGDSSSGWTAGAAAYYVGYLQPRTFLLKYNENGTITDAVDC |
| extended signal | ALDPLSETKCTLKSFTVEKGIYQTSNFRVQPTESIVRFPNITNLC |
| sequence | PFGEVFNATRFASVYAWNRKRISNCVADYSVLYNSASFSTFK |
| | CYGVSPTKLNDLCFTNVYADSFVIRGDEVRQIAPGQTGKIADY |
| | NYKLPDDFTGCVIAWNSNNLDSKVGGNYNYLYRLFRKSNLK |
| | PFERDISTEIYQAGSTPCNGVEGFNCYFPLQSYGFQPTNGVGY |
| | QPYRVVVLSFELLHAPATVCGPKKSTNLVKNKCVNFNFNGLT |
| | GTGVLTESNKKFLPFQQFGRDIADTTDAVRDPQTLEILDITPCS |
| | FGGVSVITPGTNTSNQVAVLYQDVNCTEVPVAIHADQLTPTW |
| | RVYSTGSNVFQTRAGCLIGAEHVNNSYECDIPIGAGICASYQT |
| | QTNSPGSASSVASQSIIAYTMSLGAENSVAYSNNSIAIPTNFTIS |
| | VTTEILPVSMTKTSVDCTMYICGDSTECSNLLLQYGSFCTQLN |
| | RALTGIAVEQDKNTQEVFAQVKQIYKTPPIKDFGGFNFSQILPD |
| | PSKPSKRSPIEDLLFNKVTLADAGFIKQYGDCLGDIAARDLICA |
| | QKFNGLTVLPPLLTDEMIAQYTSALLAGTITSGWTFGAGPALQ |
| | IPFPMQMAYRFNGIGVTQNVLYENQKLIANQFNSAIGKIQDSL |
| | SSTPSALGKLQDVVNQNAQALNTLVKQLSSNFGAISSVLNDIL |
| | SRLDPPEAEVQIDRLITGRLQSLQTYVTQQLIRAAEIRASANLA |
| | ATKMSECVLGQSKRVDFCGKGYHLMSFPQSAPHGVVFLHVT |
| | YVPAQEKNFTTAPAICHDGKAHFPREGVFVSNGTHWFVTQRN |
| | FYEPQIITTDNTFVSGNCDVVIGIVNNTVYDPLQPELDSFKEEL |
| | DKYFKNHTSPDVDLGDISGINASVVNIQKEIDRLNEVAKNLNE |
| | SLIDLQELGKYEQYIKWPWYIWLGFIAGLIAIVMVTIMLCCMT |
| | SCCSCLKGCCSCGSCCKFDEDDSEPVLKGVALAYT |
| Optimized nucleotide | (SEQ ID NO: 150) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to contain the | CACCAACTCCTTCACCAGAGGCGTGTATTACCCAGACAAGG |
| H69-, V70-, Y144-, | TGTTTAGAAGCAGCGTGCTGCACTCTACCCAGGACCTCTTT |
| N501Y, A570D, D614G, | CTGCCCTTTTTCAGCAACGTGACATGGTTTCACGCAATTTCC |
| P681H, T716I, S982A | GGCACTAATGGCACAAAGCGGTTCGACAATCCAGTCCTGC |
| and D1118H mutations | CTTTCAACGATGGCGTCTACTTTGCATCTACTGAGAAATCC |
| | AATATCATTAGGGGATGGATCTTCGGCACAACCCTGGATTC |
| | TAAGACCCAGAGCCTGCTGATCGTCAACAACGCCACAAAC |
| | GTGGTCATTAAGGTTTGCGAGTTTCAGTTCTGTAACGATCC |
| | TTTTCTGGGCGTTTACCATAAGAACAATAAGAGCTGGATGG |
| | AGTCCGAGTTTAGAGTGTATAGCTCTGCAAATAATTGTACC |
| | TTTGAGTACGTGAGCCAGCCCTTTCTGATGGACCTGGAGGG |
| | AAAACAAGGAAACTTCAAAAACCTGCGGGAATTCGTTTTC |
| | AAAAACATCGACGGCTATTTCAAGATCTATAGCAAGCATA |
| | CCCCAATCAACCTCGTGAGGGACCTCCCCCAGGGCTTTAGC |
| | GCACTGGAGCCACTGGTTGACCTGCCTATCGGCATTAATAT |
| | CACAAGATTTCAGACCCTGCTGGCACTGCATAGAAGCTATC |
| | TGACCCCTGGAGACTCCTCTAGTGGGTGGACTGCCGGCGCC |
| | GCTGCCTACTATGTGGGCTATCTGCAGCCACGGACATTCCT |
| | GCTGAAATACAATGAGAACGGGACAATCACAGATGCTGTT |
| | GATTGCGCACTCGACCCCCTGTCCGAGACAAAGTGCACTCT |
| | CAAGAGCTTTACCGTCGAGAAGGGCATCTATCAGACCTCA |
| | AACTTCAGGGTGCAGCCCACAGAATCTATCGTGCGCTTCCC |
| | TAATATCACTAACCTGTGTCCTTTCGGTGAAGTGTTCAACG |
| | CCACCAGGTTTGCTAGCGTGTATGCCTGGAACAGGAAGAG |
| | GATCTCTAACTGCGTCGCCGACTATTCCGTGCTGTATAACA |
| | GCGCCTCCTTCTCCACATTCAAATGCTATGGAGTGAGCCCG |
| | ACAAAACTGAACGATCTCTGCTTTACAAATGTCTACGCCGA |
| | CTCTTTTGTGATCAGAGGGGACGAGGTCCGGCAGATCGCAC |
| | CAGGACAGACAGGCAAGATTGCTGACTACAACTATAAGCT |
| | GCCTGACGACTTCACAGGATGTGTGATCGCATGGAACTCAA |
| | ACAATCTGGACTCCAAAGTCGGGGGCAACTATAATTACCTG |
| | TATCGCCTGTTCCGGAAGTCCAACCTGAAGCCCTTCGAGAG |
| | GGACATCAGTACAGAGATCTATCAGGCTGGCTCCACCCCTT |
| | GCAATGGCGTCGAAGGCTTTAATTGTTATTTTCCCCTGCAG |
| | TCTTACGGGTTTCAGCCTACTTATGGAGTTGGGTACCAGCC |
| | ATACAGAGTGGTCGTGCTCAGCTTCGAGCTCCTGCATGCTC |
| | CAGCTACAGTTTGCGGGCCAAAGAAGTCCACTAACCTGGT |
| | GAAGAATAAGTGCGTCAACTTCAACTTTAACGGGCTCACCG |
| | GCACCGGCGTGCTGACTGAGAGCAACAAGAAGTTTCTGCC |
| | ATTTCAACAGTTTGGACGGGACATTGATGACACCACCGAT |
| | GCCGTTCGGGATCCACAGACCCTGGAAATTCTGGACATTAC |
| | ACCGTGCAGCTTCGGGGGCGTGAGCGTGATCACACCCGGA |
| | ACCAATACAAGCAACCAGGTTGCCGTCCTGTATCAGGGAG |
| | TCAATTGCACAGAAGTGCCAGTTGCTATCCACGCAGACCAG |
| | CTGACTCCCACATGGCGGGTGTATAGCACCGGATCCAACGT |
| | GTTTCAGACCCGCGCCGGATGTCTCATTGGGGCCGAGCACG |
| | TGAATAACAGCTACGAGTGCGACATCCCCATTGGCGCCGG |
| | CATTTGTGCGTCTTACCAGACTCAGACCAACTCTCATAGAA |
| | GGGCCAGGTCCGTTGCTAGTCAGTCTATTATTGCCTATACC |
| | ATGAGCCTCGGAGCTGAGAATAGCGTGGCCTACTCCAATA |
| | ATTCCATCGCAATCCCTATAAACTTCACTATTTCTGTGACC |
| | ACCGAGATCCTGCCTGTGTCTATGACTAAGACTAGCGTTGA |
| | TTGTACCATGTATATTTGTGGCGACTCTACCGAATGTTCTA |
| | ACCTGCTGCTTCAGTACGGCTCATTTTGCACACAGCTGAAC |
| | AGAGCCCTGACTGGGATCGCTGTGGAGCAGGACAAGAACA |
| | CACAGGAGGTGTTTGCACAGGTGAAGCAGATCTATAAGAC |
| | CCCTCCTATTAAGGATTTCGGCGGATTCAATTTCTCACAGA |
| | TTCTGCCAGACCCCAGTAAGCCTTCCAAGAGGAGCTTCATC |
| | GAGGATCTCCTGTTTAACAAGGTGACCCTGGCAGACGCCG |
| | GCTTTATTAAGCAATATGGGGATTGCCTGGGCGACATTGCT |
| | GCCAGAGACCTGATTTGCGCCCAGAAATTCAATGGCCTCAC |
| | AGTGCTGCCACCTCTGCTGACCGACGAGATGATCGCTCAAT |
| | ACACTAGCGCACTGCTGGCCGGAACCATCACATCAGGCTG |
| | GACCTTCGGGGCCGGAGCAGCACTGCAGATTCCATTCGCCA |
| | TGCAGATGGCCTATAGATTCAACGGCATTGGCGTCACACAG |
| | AACGTGCTGTACGAAAACCAGAAGCTCATCGCTAACCAGT |
| | TTAATTCCGCAATTGGAAAGATCCAAGATTCACTCAGCTCA |
| | ACCGCCTCTGCACTCGGAAAGCTGCAGGACGTGGTCAACC |
| | AGAATGCTCAGGCCCTGAACACACTCGTCAAGCAGCTGTCC |
| | TCTAACTTTGGCGCTATCAGCTCCGTTCTGAACGACATTCT |
| | GGCACGCCTGGATAAGGTGGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTCACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGTATATCAA |
| | ATGGCCCTGGTACATTTGGCTGGGGTTTATCGCCGGACTGA |
| | TTGCCATCGTCATGGTGACCATCATGCTGTGTTGCATGACC |
| | TCCTGTTGTTCCTGTCTGAAGGGCTGCTGTAGTTGCGGCTCT |
| | TGCTGTAAATTCGACGAAGATGATAGCGAGCCCGTGCTGA |
| | AGGGCGTGAAGCTGCATTATACCTGA |
| SARS-CoV-2 S protein | (SEQ ID NO: 151) |
| mutated to contain the | MFVFLVLLPLVSSQCVNLTTRTQLPPAYTNSFTRGVYYPDKVF |
| H69-, V70-, Y144-, | RSSVLHSTQDLFLPFFSNVTWFHAISGTNGTKRFDNPVLPFND |
| N501Y, A570D, D614G, | GVYFASTEKSNIIRGWIFGTTLDSKTQSLLIVNNATNVVIKVCE |
| P681H, T716I, S982A | FQFCNDPFLGVYHKNNKSWMESEFRVYSSANNCTFEYVSQPF |
| and D1118H mutations | LMDLEGKQGNFKNLREFVFKNIDGYFKIYSKHTPINLVRDLPQ |
| | GFSALEPLVDLPIGINITRFQTLLALHRSYLTPGDSSSGWTAGA |
| | AAYYVGYLQPRTFLLKYNENGTITDAVDCALDPLSETKCTLK |
| | SFTVEKGIYQTSNFRVQPTESIVRFPNITNLCPFGEVFNATRFAS |
| | VYAWNRKRISNCVADYSVLYNSASFSTFKCYGVSPTKLNDLC |
| | FTNVYADSFVIRGDEVRQIAPGQTGKIADYNYKLPDDFTGCVI |
| | AWNSNNLDSKVGGNYNYLYRLFRKSNLKPFERDISTEIYQAG |
| | STPCNGVEGFNCYFPLQSYGFQPTYGVGYQPYRVVVLSFELL |
| | HAPATVCGPKKSTNLVKNKCVNFNFNGLTGTGVLTESNKKFL |
| | PFQQFGRDIDDTTDAVRDPQTLEILDITPCSFGGVSVITPGTNTS |
| | NQVAVLYQGVNCTEVPVAIHADQLTPTWRVYSTGSNVFQTR |
| | AGCLIGAEHVNNSYECDIPIGAGICASYQTQTNSHRRARSVAS |
| | QSIIAYTMSLGAENSVAYSNNSIAIPINFTISVTTEILPVSMTKTS |
| | VDCTMYICGDSTECSNLLLQYGSFCTQLNRALTGIAVEQDKN |
| | TQEVFAQVKQIYKTPPIKDFGGFNFSQILPDPSKPSKRSFIEDLL |
| | FNKVTLADAGFIKQYGDCLGDIAARDLICAQKFNGLTVLPPLL |
| | TDEMIAQYTSALLAGTITSGWTFGAGAALQIPFAMQMAYRFN |
| | GIGVTQNVLYENQKLIANQFNSAIGKIQDSLSSTASALGKLQD |
| | VVNQNAQALNTLVKQLSSNFGAISSVLNDILARLDKVEAEVQI |
| | DRLITGRLQSLQTYVTQQLIRAAEIRASANLAATKMSECVLGQ |
| | SKRVDFCGKGYHLMSFPQSAPHGVVFLHVTYVPAQEKNFTTA |
| | PAICHDGKAHFPREGVFVSNGTHWFVTQRNFYEPQIITTHNTF |
| | VSGNCDVVIGIVNNTVYDPLQPELDSFKEELDKYFKNHTSPDV |
| | DLGDISGINASVVNIQKEIDRLNEVAKNLNESLIDLQELGKYEQ |
| | YIKWPWYIWLGFIAGLIAIVMVTIMLCCMTSCCSCLKGCCSCG |
| | SCCKFDEDDSEPVLKGVKLHYT* |
| Optimized nucleotide | (SEQ ID NO: 152) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| with residues 986 and | CACCAACTCCTTCACCAGAGGCGTGTATTACCCAGACAAGG |
| 987 mutated to proline | TGTTTAGAAGCAGCGTGCTGCACTCTACCCAGGACCTCTTT |
| and which contains the | CTGCCCTTTTTCAGCAACGTGACATGGTTTCACGCAATTTCC |
| H69-, V70-, Y144-, | GGCACTAATGGCACAAAGCGGTTCGACAATCCAGTCCTGC |
| N501Y, A570D, D614G, | CTTTCAACGATGGCGTCTACTTTGCATCTACTGAGAAATCC |
| P681H, T716I, S982A | AATATCATTAGGGGATGGATCTTCGGCACAACCCTGGATTC |
| and D1118H mutations | TAAGACCCAGAGCCTGCTGATCGTCAACAACGCCACAAAC |
| | GTGGTCATTAAGGTTTGCGAGTTTCAGTTCTGTAACGATCC |
| | TTTTCTGGGCGTTTACCATAAGAACAATAAGAGCTGGATGG |
| | AGTCCGAGTTTAGAGTGTATAGCTCTGCAAATAATTGTACC |
| | TTTGAGTACGTGAGCCAGCCCTTTCTGATGGACCTGGAGGG |
| | AAAACAAGGAAACTTCAAAAACCTGCGGGAATTCGTTTTC |
| | AAAAACATCGACGGCTATTTCAAGATCTATAGCAAGCATA |
| | CCCCAATCAACCTCGTGAGGGACCTCCCCCAGGGCTTTAGC |
| | GCACTGGAGCCACTGGTTGACCTGCCTATCGGCATTAATAT |
| | CACAAGATTTCAGACCCTGCTGGCACTGCATAGAAGCTATC |
| | TGACCCCTGGAGACTCCTCTAGTGGGTGGACTGCCGGCGCC |
| | GCTGCCTACTATGTGGGCTATCTGCAGCCACGGACATTCCT |
| | GCTGAAATACAATGAGAACGGGACAATCACAGATGCTGTT |
| | GATTGCGCACTCGACCCCCTGTCCGAGACAAAGTGCACTCT |
| | CAAGAGCTTTACCGTCGAGAAGGGCATCTATCAGACCTCA |
| | AACTTCAGGGTGCAGCCCACAGAATCTATCGTGCGCTTCCC |
| | TAATATCACTAACCTGTGTCCTTTCGGTGAAGTGTTCAACG |
| | CCACCAGGTTTGCTAGCGTGTATGCCTGGAACAGGAAGAG |
| | GATCTCTAACTGCGTCGCCGACTATTCCGTGCTGTATAACA |
| | GCGCCTCCTTCTCCACATTCAAATGCTATGGAGTGAGCCCG |
| | ACAAAACTGAACGATCTCTGCTTTACAAATGTCTACGCCGA |
| | CTCTTTTGTGATCAGAGGGGACGAGGTCCGGCAGATCGCAC |
| | CAGGACAGACAGGCAAGATTGCTGACTACAACTATAAGCT |
| | GCCTGACGACTTCACAGGATGTGTGATCGCATGGAACTCAA |
| | ACAATCTGGACTCCAAAGTCGGGGGCAACTATAATTACCTG |
| | TATCGCCTGTTCCGGAAGTCCAACCTGAAGCCCTTCGAGAG |
| | GGACATCAGTACAGAGATCTATCAGGCTGGCTCCACCCCTT |
| | GCAATGGCGTCGAAGGCTTTAATTGTTATTTTCCCCTGCAG |
| | TCTTACGGGTTTCAGCCTACTTATGGAGTTGGGTACCAGCC |
| | ATACAGAGTGGTCGTGCTCAGCTTCGAGCTCCTGCATGCTC |
| | CAGCTACAGTTTGCGGGCCAAAGAAGTCCACTAACCTGGT |
| | GAAGAATAAGTGCGTCAACTTCAACTTTAACGGGCTCACCG |
| | GCACCGGCGTGCTGACTGAGAGCAACAAGAAGTTTCTGCC |
| | ATTTCAACAGTTTGGACGGGACATTGATGACACCACCGAT |
| | GCCGTTCGGGATCCACAGACCCTGGAAATTCTGGACATTAC |
| | ACCGTGCAGCTTCGGGGGCGTGAGCGTGATCACACCCGGA |
| | ACCAATACAAGCAACCAGGTTGCCGTCCTGTATCAGGGAG |
| | TCAATTGCACAGAAGTGCCAGTTGCTATCCACGCAGACCAG |
| | CTGACTCCCACATGGCGGGTGTATAGCACCGGATCCAACGT |
| | GTTTCAGACCCGCGCCGGATGTCTCATTGGGGCCGAGCACG |
| | TGAATAACAGCTACGAGTGCGACATCCCCATTGGCGCCGG |
| | CATTTGTGCGTCTTACCAGACTCAGACCAACTCTCATAGAA |
| | GGGCCAGGTCCGTTGCTAGTCAGTCTATTATTGCCTATACC |
| | ATGAGCCTCGGAGCTGAGAATAGCGTGGCCTACTCCAATA |
| | ATTCCATCGCAATCCCTATAAACTTCACTATTTCTGTGACC |
| | ACCGAGATCCTGCCTGTGTCTATGACTAAGACTAGCGTTGA |
| | TTGTACCATGTATATTTGTGGCGACTCTACCGAATGTTCTA |
| | ACCTGCTGCTTCAGTACGGCTCATTTTGCACACAGCTGAAC |
| | AGAGCCCTGACTGGGATCGCTGTGGAGCAGGACAAGAACA |
| | CACAGGAGGTGTTTGCACAGGTGAAGCAGATCTATAAGAC |
| | CCCTCCTATTAAGGATTTCGGCGGATTCAATTTCTCACAGA |
| | TTCTGCCAGACCCCAGTAAGCCTTCCAAGAGGAGCTTCATC |
| | GAGGATCTCCTGTTTAACAAGGTGACCCTGGCAGACGCCG |
| | GCTTTATTAAGCAATATGGGGATTGCCTGGGCGACATTGCT |
| | GCCAGAGACCTGATTTGCGCCCAGAAATTCAATGGCCTCAC |
| | AGTGCTGCCACCTCTGCTGACCGACGAGATGATCGCTCAAT |
| | ACACTAGCGCACTGCTGGCCGGAACCATCACATCAGGCTG |
| | GACCTTCGGGGCCGGAGCAGCACTGCAGATTCCATTCGCCA |
| | TGCAGATGGCCTATAGATTCAACGGCATTGGCGTCACACAG |
| | AACGTGCTGTACGAAAACCAGAAGCTCATCGCTAACCAGT |
| | TTAATTCCGCAATTGGAAAGATCCAAGATTCACTCAGCTCA |
| | ACCGCCTCTGCACTCGGAAAGCTGCAGGACGTGGTCAACC |
| | AGAATGCTCAGGCCCTGAACACACTCGTCAAGCAGCTGTCC |
| | TCTAACTTTGGCGCTATCAGCTCCGTTCTGAACGACATTCT |
| | GGCACGCCTGGATCCCCCAGAGGCTGAAGTCCAGATTGAC |
| | CGCCTGATTACCGGCCGGCTGCAGTCTCTGCAAACATACGT |
| | GACCCAGCAGCTGATCAGAGCAGCCGAGATCCGGGCATCC |
| | GCAAATCTGGCAGCAACTAAGATGAGCGAATGCGTGCTGG |
| | GCCAGTCCAAGCGGGTGGACTTTTGTGGCAAGGGCTACCA |
| | CCTGATGAGCTTCCCCCAGAGCGCCCCACATGGCGTTGTTT |
| | TTCTGCACGTGACCTATGTCCCTGCTCAGGAAAAGAACTTT |
| | ACAACTGCTCCTGCTATCTGCCATGACGGCAAGGCCCACTT |
| | CCCACGGGAGGGAGTGTTTGTGTCCAATGGCACACACTGGT |
| | TCGTGACCCAGAGGAACTTCTATGAACCCCAGATCATCACC |
| | ACTCACAATACCTTCGTGTCTGGAAATTGCGACGTCGTGAT |
| | CGGCATCGTTAACAACACCGTGTACGACCCTCTCCAGCCAG |
| | AGCTGGACTCCTTTAAGGAGGAACTGGATAAGTATTTTAAG |
| | AACCACACAAGCCCAGATGTGGATCTCGGGGACATCTCCG |
| | GAATTAACGCCTCCGTGGTGAATATCCAGAAGGAGATTGA |
| | CCGCCTAAATGAAGTTGCCAAGAACCTCAATGAGTCTCTGA |
| | TTGATCTGCAGGAACTGGGCAAGTATGAGCAGTATATCAA |
| | ATGGCCCTGGTACATTTGGCTGGGGTTTATCGCCGGACTGA |
| | TTGCCATCGTCATGGTGACCATCATGCTGTGTTGCATGACC |
| | TCCTGTTGTTCCTGTCTGAAGGGCTGCTGTAGTTGCGGCTCT |
| | TGCTGTAAATTCGACGAAGATGATAGCGAGCCCGTGCTGA |
| | AGGGCGTGAAGCTGCATTATACCTGA |
| SARS-CoV-2 S protein | (SEQ ID NO: 153) |
| with residues 986 and | MFVFLVLLPLVSSQCVNLTTRTQLPPAYTNSFTRGVYYPDKVF |
| 987 mutated to proline | RSSVLHSTQDLFLPFFSNVTWFHAISGTNGTKRFDNPVLPFND |
| and which contains the | GVYFASTEKSNIIRGWIFGTTLDSKTQSLLIVNNATNVVIKVCE |
| H69-, V70-, Y144-, | FQFCNDPFLGVYHKNNKSWMESEFRVYSSANNCTFEYVSQPF |
| N501Y, A570D, D614G, | LMDLEGKQGNFKNLREFVFKNIDGYFKIYSKHTPINLVRDLPQ |
| P681H, T716I, S982A | GFSALEPLVDLPIGINITRFQTLLALHRSYLTPGDSSSGWTAGA |
| and D1118H mutations | AAYYVGYLQPRTFLLKYNENGTITDAVDCALDPLSETKCTLK |
| | SFTVEKGIYQTSNFRVQPTESIVRFPNITNLCPFGEVFNATRFAS |
| | VYAWNRKRISNCVADYSVLYNSASFSTFKCYGVSPTKLNDLC |
| | FTNVYADSFVIRGDEVRQIAPGQTGKIADYNYKLPDDFTGCVI |
| | AWNSNNLDSKVGGNYNYLYRLFRKSNLKPFERDISTEIYQAG |
| | STPCNGVEGFNCYFPLQSYGFQPTYGVGYQPYRVVVLSFELL |
| | HAPATVCGPKKSTNLVKNKCVNFNFNGLTGTGVLTESNKKFL |
| | PFQQFGRDIDDTTDAVRDPQTLEILDITPCSFGGVSVITPGTNTS |
| | NQVAVLYQGVNCTEVPVAIHADQLTPTWRVYSTGSNVFQTR |
| | AGCLIGAEHVNNSYECDIPIGAGICASYQTQTNSHRRARSVAS |
| | QSIIAYTMSLGAENSVAYSNNSIAIPINFTISVTTEILPVSMTKTS |
| | VDCTMYICGDSTECSNLLLQYGSFCTQLNRALTGIAVEQDKN |
| | TQEVFAQVKQIYKTPPIKDFGGFNFSQILPDPSKPSKRSFIEDLL |
| | FNKVTLADAGFIKQYGDCLGDIAARDLICAQKFNGLTVLPPLL |
| | TDEMIAQYTSALLAGTITSGWTFGAGAALQIPFAMQMAYRFN |
| | GIGVTQNVLYENQKLIANQFNSAIGKIQDSLSSTASALGKLQD |
| | VVNQNAQALNTLVKQLSSNFGAISSVLNDILARLDPPEAEVQI |
| | DRLITGRLQSLQTYVTQQLIRAAEIRASANLAATKMSECVLGQ |
| | SKRVDFCGKGYHLMSFPQSAPHGVVFLHVTYVPAQEKNFTTA |
| | PAICHDGKAHFPREGVFVSNGTHWFVTQRNFYEPQIITTHNTF |
| | VSGNCDVVIGIVNNTVYDPLQPELDSFKEELDKYFKNHTSPDV |
| | DLGDISGINASVVNIQKEIDRLNEVAKNLNESLIDLQELGKYEQ |
| | YIKWPWYIWLGFIAGLIAIVMVTIMLCCMTSCCSCLKGCCSCG |
| | SCCKFDEDDSEPVLKGVKLHYT |
| Optimized nucleotide | (SEQ ID NO: 154) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove the | CACCAACTCCTTCACCAGAGGCGTGTATTACCCAGACAAGG |
| furin cleavage site | TGTTTAGAAGCAGCGTGCTGCACTCTACCCAGGACCTCTTT |
| required for activation | CTGCCCTTTTTCAGCAACGTGACATGGTTTCACGCAATTTCC |
| and which contains the | GGCACTAATGGCACAAAGCGGTTCGACAATCCAGTCCTGC |
| H69-, V70-, Y144-, | CTTTCAACGATGGCGTCTACTTTGCATCTACTGAGAAATCC |
| N501Y, A570D, D614G, | AATATCATTAGGGGATGGATCTTCGGCACAACCCTGGATTC |
| P681H, T716I, S982A | TAAGACCCAGAGCCTGCTGATCGTCAACAACGCCACAAAC |
| and D1118H mutations | GTGGTCATTAAGGTTTGCGAGTTTCAGTTCTGTAACGATCC |
| | TTTTCTGGGCGTTTACCATAAGAACAATAAGAGCTGGATGG |
| | AGTCCGAGTTTAGAGTGTATAGCTCTGCAAATAATTGTACC |
| | TTTGAGTACGTGAGCCAGCCCTTTCTGATGGACCTGGAGGG |
| | AAAACAAGGAAACTTCAAAAACCTGCGGGAATTCGTTTTC |
| | AAAAACATCGACGGCTATTTCAAGATCTATAGCAAGCATA |
| | CCCCAATCAACCTCGTGAGGGACCTCCCCCAGGGCTTTAGC |
| | GCACTGGAGCCACTGGTTGACCTGCCTATCGGCATTAATAT |
| | CACAAGATTTCAGACCCTGCTGGCACTGCATAGAAGCTATC |
| | TGACCCCTGGAGACTCCTCTAGTGGGTGGACTGCCGGCGCC |
| | GCTGCCTACTATGTGGGCTATCTGCAGCCACGGACATTCCT |
| | GCTGAAATACAATGAGAACGGGACAATCACAGATGCTGTT |
| | GATTGCGCACTCGACCCCCTGTCCGAGACAAAGTGCACTCT |
| | CAAGAGCTTTACCGTCGAGAAGGGCATCTATCAGACCTCA |
| | AACTTCAGGGTGCAGCCCACAGAATCTATCGTGCGCTTCCC |
| | TAATATCACTAACCTGTGTCCTTTCGGTGAAGTGTTCAACG |
| | CCACCAGGTTTGCTAGCGTGTATGCCTGGAACAGGAAGAG |
| | GATCTCTAACTGCGTCGCCGACTATTCCGTGCTGTATAACA |
| | GCGCCTCCTTCTCCACATTCAAATGCTATGGAGTGAGCCCG |
| | ACAAAACTGAACGATCTCTGCTTTACAAATGTCTACGCCGA |
| | CTCTTTTGTGATCAGAGGGGACGAGGTCCGGCAGATCGCAC |
| | CAGGACAGACAGGCAAGATTGCTGACTACAACTATAAGCT |
| | GCCTGACGACTTCACAGGATGTGTGATCGCATGGAACTCAA |
| | ACAATCTGGACTCCAAAGTCGGGGGCAACTATAATTACCTG |
| | TATCGCCTGTTCCGGAAGTCCAACCTGAAGCCCTTCGAGAG |
| | GGACATCAGTACAGAGATCTATCAGGCTGGCTCCACCCCTT |
| | GCAATGGCGTCGAAGGCTTTAATTGTTATTTTCCCCTGCAG |
| | TCTTACGGGTTTCAGCCTACTTATGGAGTTGGGTACCAGCC |
| | ATACAGAGTGGTCGTGCTCAGCTTCGAGCTCCTGCATGCTC |
| | CAGCTACAGTTTGCGGGCCAAAGAAGTCCACTAACCTGGT |
| | GAAGAATAAGTGCGTCAACTTCAACTTTAACGGGCTCACCG |
| | GCACCGGCGTGCTGACTGAGAGCAACAAGAAGTTTCTGCC |
| | ATTTCAACAGTTTGGACGGGACATTGATGACACCACCGAT |
| | GCCGTTCGGGATCCACAGACCCTGGAAATTCTGGACATTAC |
| | ACCGTGCAGCTTCGGGGGCGTGAGCGTGATCACACCCGGA |
| | ACCAATACAAGCAACCAGGTTGCCGTCCTGTATCAGGGAG |
| | TCAATTGCACAGAAGTGCCAGTTGCTATCCACGCAGACCAG |
| | CTGACTCCCACATGGCGGGTGTATAGCACCGGATCCAACGT |
| | GTTTCAGACCCGCGCCGGATGTCTCATTGGGGCCGAGCACG |
| | TGAATAACAGCTACGAGTGCGACATCCCCATTGGCGCCGG |
| | CATTTGTGCGTCTTACCAGACTCAGACCAACTCTCATGGCT |
| | CCGCCTCTTCCGTTGCTAGTCAGTCTATTATTGCCTATACCA |
| | TGAGCCTCGGAGCTGAGAATAGCGTGGCCTACTCCAATAAT |
| | TCCATCGCAATCCCTATAAACTTCACTATTTCTGTGACCAC |
| | CGAGATCCTGCCTGTGTCTATGACTAAGACTAGCGTTGATT |
| | GTACCATGTATATTTGTGGCGACTCTACCGAATGTTCTAAC |
| | CTGCTGCTTCAGTACGGCTCATTTTGCACACAGCTGAACAG |
| | AGCCCTGACTGGGATCGCTGTGGAGCAGGACAAGAACACA |
| | CAGGAGGTGTTTGCACAGGTGAAGCAGATCTATAAGACCC |
| | CTCCTATTAAGGATTTCGGCGGATTCAATTTCTCACAGATT |
| | CTGCCAGACCCCAGTAAGCCTTCCAAGAGGAGCTTCATCGA |
| | GGATCTCCTGTTTAACAAGGTGACCCTGGCAGACGCCGGCT |
| | TTATTAAGCAATATGGGGATTGCCTGGGCGACATTGCTGCC |
| | AGAGACCTGATTTGCGCCCAGAAATTCAATGGCCTCACAGT |
| | GCTGCCACCTCTGCTGACCGACGAGATGATCGCTCAATACA |
| | CTAGCGCACTGCTGGCCGGAACCATCACATCAGGCTGGAC |
| | CTTCGGGGCCGGAGCAGCACTGCAGATTCCATTCGCCATGC |
| | AGATGGCCTATAGATTCAACGGCATTGGCGTCACACAGAA |
| | CGTGCTGTACGAAAACCAGAAGCTCATCGCTAACCAGTTTA |
| | ATTCCGCAATTGGAAAGATCCAAGATTCACTCAGCTCAACC |
| | GCCTCTGCACTCGGAAAGCTGCAGGACGTGGTCAACCAGA |
| | ATGCTCAGGCCCTGAACACACTCGTCAAGCAGCTGTCCTCT |
| | AACTTTGGCGCTATCAGCTCCGTTCTGAACGACATTCTGGC |
| | ACGCCTGGATAAGGTGGAGGCTGAAGTCCAGATTGACCGC |
| | CTGATTACCGGCCGGCTGCAGTCTCTGCAAACATACGTGAC |
| | CCAGCAGCTGATCAGAGCAGCCGAGATCCGGGCATCCGCA |
| | AATCTGGCAGCAACTAAGATGAGCGAATGCGTGCTGGGCC |
| | AGTCCAAGCGGGTGGACTTTTGTGGCAAGGGCTACCACCTG |
| | ATGAGCTTCCCCCAGAGCGCCCCACATGGCGTTGTTTTTCT |
| | GCACGTGACCTATGTCCCTGCTCAGGAAAAGAACTTTACAA |
| | CTGCTCCTGCTATCTGCCATGACGGCAAGGCCCACTTCCCA |
| | CGGGAGGGAGTGTTTGTGTCCAATGGCACACACTGGTTCGT |
| | GACCCAGAGGAACTTCTATGAACCCCAGATCATCACCACTC |
| | ACAATACCTTCGTGTCTGGAAATTGCGACGTCGTGATCGGC |
| | ATCGTTAACAACACCGTGTACGACCCTCTCCAGCCAGAGCT |
| | GGACTCCTTTAAGGAGGAACTGGATAAGTATTTTAAGAACC |
| | ACACAAGCCCAGATGTGGATCTCGGGGACATCTCCGGAAT |
| | TAACGCCTCCGTGGTGAATATCCAGAAGGAGATTGACCGC |
| | CTAAATGAAGTTGCCAAGAACCTCAATGAGTCTCTGATTGA |
| | TCTGCAGGAACTGGGCAAGTATGAGCAGTATATCAAATGG |
| | CCCTGGTACATTTGGCTGGGGTTTATCGCCGGACTGATTGC |
| | CATCGTCATGGTGACCATCATGCTGTGTTGCATGACCTCCT |
| | GTTGTTCCTGTCTGAAGGGCTGCTGTAGTTGCGGCTCTTGCT |
| | GTAAATTCGACGAAGATGATAGCGAGCCCGTGCTGAAGGG |
| | CGTGAAGCTGCATTATACCTGA |
| SARS-CoV-2 S protein | (SEQ ID NO: 155) |
| mutated to remove the | MFVFLVLLPLVSSQCVNLTTRTQLPPAYTNSFTRGVYYPDKVF |
| furin cleavage site | RSSVLHSTQDLFLPFFSNVTWFHAISGTNGTKRFDNPVLPFND |
| required for activation | GVYFASTEKSNIIRGWIFGTTLDSKTQSLLIVNNATNVVIKVCE |
| and which contains the | FQFCNDPFLGVYHKNNKSWMESEFRVYSSANNCTFEYVSQPF |
| H69-, V70-, Y144-, | LMDLEGKQGNFKNLREFVFKNIDGYFKIYSKHTPINLVRDLPQ |
| N501Y, A570D, D614G, | GFSALEPLVDLPIGINITRFQTLLALHRSYLTPGDSSSGWTAGA |
| P681H, T716I, S982A | AAYYVGYLQPRTFLLKYNENGTITDAVDCALDPLSETKCTLK |
| and D1118H mutations | SFTVEKGIYQTSNFRVQPTESIVRFPNITNLCPFGEVFNATRFAS |
| | VYAWNRKRISNCVADYSVLYNSASFSTFKCYGVSPTKLNDLC |
| | FTNVYADSFVIRGDEVRQIAPGQTGKIADYNYKLPDDFTGCVI |
| | AWNSNNLDSKVGGNYNYLYRLFRKSNLKPFERDISTEIYQAG |
| | STPCNGVEGFNCYFPLQSYGFQPTYGVGYQPYRVVVLSFELL |
| | HAPATVCGPKKSTNLVKNKCVNFNFNGLTGTGVLTESNKKFL |
| | PFQQFGRDIDDTTDAVRDPQTLEILDITPCSFGGVSVITPGTNTS |
| | NQVAVLYQGVNCTEVPVAIHADQLTPTWRVYSTGSNVFQTR |
| | AGCLIGAEHVNNSYECDIPIGAGICASYQTQTNSHGSASSVAS |
| | QSIIAYTMSLGAENSVAYSNNSIAIPINFTISVTTEILPVSMTKTS |
| | VDCTMYICGDSTECSNLLLQYGSFCTQLNRALTGIAVEQDKN |
| | TQEVFAQVKQIYKTPPIKDFGGFNFSQILPDPSKPSKRSFIEDLL |
| | FNKVTLADAGFIKQYGDCLGDIAARDLICAQKFNGLTVLPPLL |
| | TDEMIAQYTSALLAGTITSGWTFGAGAALQIPFAMQMAYRFN |
| | GIGVTQNVLYENQKLIANQFNSAIGKIQDSLSSTASALGKLQD |
| | VVNQNAQALNTLVKQLSSNFGAISSVLNDILARLDKVEAEVQI |
| | DRLITGRLQSLQTYVTQQLIRAAEIRASANLAATKMSECVLGQ |
| | SKRVDFCGKGYHLMSFPQSAPHGVVFLHVTYVPAQEKNFTTA |
| | PAICHDGKAHFPREGVFVSNGTHWFVTQRNFYEPQIITTHNTF |
| | VSGNCDVVIGIVNNTVYDPLQPELDSFKEELDKYFKNHTSPDV |
| | DLGDISGINASVVNIQKEIDRLNEVAKNLNESLIDLQELGKYEQ |
| | YIKWPWYIWLGFIAGLIAIVMVTIMLCCMTSCCSCLKGCCSCG |
| | SCCKFDEDDSEPVLKGVKLHYT |
| Optimized nucleotide | (SEQ ID NO: 156) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove a | CACCAACTCCTTCACCAGAGGCGTGTATTACCCAGACAAGG |
| furin cleavage site and | TGTTTAGAAGCAGCGTGCTGCACTCTACCCAGGACCTCTTT |
| to replace residues 986 | CTGCCCTTTTTCAGCAACGTGACATGGTTTCACGCAATTTCC |
| and 987 with proline | GGCACTAATGGCACAAAGCGGTTCGACAATCCAGTCCTGC |
| and which contains the | CTTTCAACGATGGCGTCTACTTTGCATCTACTGAGAAATCC |
| H69-, V70-, Y144-, | AATATCATTAGGGGATGGATCTTCGGCACAACCCTGGATTC |
| N501Y, A570D, D614G, | TAAGACCCAGAGCCTGCTGATCGTCAACAACGCCACAAAC |
| P681H, T716I, S982A | GTGGTCATTAAGGTTTGCGAGTTTCAGTTCTGTAACGATCC |
| and D1118H mutations | TTTTCTGGGCGTTTACCATAAGAACAATAAGAGCTGGATGG |
| | AGTCCGAGTTTAGAGTGTATAGCTCTGCAAATAATTGTACC |
| | TTTGAGTACGTGAGCCAGCCCTTTCTGATGGACCTGGAGGG |
| | AAAACAAGGAAACTTCAAAAACCTGCGGGAATTCGTTTTC |
| | AAAAACATCGACGGCTATTTCAAGATCTATAGCAAGCATA |
| | CCCCAATCAACCTCGTGAGGGACCTCCCCCAGGGCTTTAGC |
| | GCACTGGAGCCACTGGTTGACCTGCCTATCGGCATTAATAT |
| | CACAAGATTTCAGACCCTGCTGGCACTGCATAGAAGCTATC |
| | TGACCCCTGGAGACTCCTCTAGTGGGTGGACTGCCGGCGCC |
| | GCTGCCTACTATGTGGGCTATCTGCAGCCACGGACATTCCT |
| | GCTGAAATACAATGAGAACGGGACAATCACAGATGCTGTT |
| | GATTGCGCACTCGACCCCCTGTCCGAGACAAAGTGCACTCT |
| | CAAGAGCTTTACCGTCGAGAAGGGCATCTATCAGACCTCA |
| | AACTTCAGGGTGCAGCCCACAGAATCTATCGTGCGCTTCCC |
| | TAATATCACTAACCTGTGTCCTTTCGGTGAAGTGTTCAACG |
| | CCACCAGGTTTGCTAGCGTGTATGCCTGGAACAGGAAGAG |
| | GATCTCTAACTGCGTCGCCGACTATTCCGTGCTGTATAACA |
| | GCGCCTCCTTCTCCACATTCAAATGCTATGGAGTGAGCCCG |
| | ACAAAACTGAACGATCTCTGCTTTACAAATGTCTACGCCGA |
| | CTCTTTTGTGATCAGAGGGGACGAGGTCCGGCAGATCGCAC |
| | CAGGACAGACAGGCAAGATTGCTGACTACAACTATAAGCT |
| | GCCTGACGACTTCACAGGATGTGTGATCGCATGGAACTCAA |
| | ACAATCTGGACTCCAAAGTCGGGGGCAACTATAATTACCTG |
| | TATCGCCTGTTCCGGAAGTCCAACCTGAAGCCCTTCGAGAG |
| | GGACATCAGTACAGAGATCTATCAGGCTGGCTCCACCCCTT |
| | GCAATGGCGTCGAAGGCTTTAATTGTTATTTTCCCCTGCAG |
| | TCTTACGGGTTTCAGCCTACTTATGGAGTTGGGTACCAGCC |
| | ATACAGAGTGGTCGTGCTCAGCTTCGAGCTCCTGCATGCTC |
| | CAGCTACAGTTTGCGGGCCAAAGAAGTCCACTAACCTGGT |
| | GAAGAATAAGTGCGTCAACTTCAACTTTAACGGGCTCACCG |
| | GCACCGGCGTGCTGACTGAGAGCAACAAGAAGTTTCTGCC |
| | ATTTCAACAGTTTGGACGGGACATTGATGACACCACCGAT |
| | GCCGTTCGGGATCCACAGACCCTGGAAATTCTGGACATTAC |
| | ACCGTGCAGCTTCGGGGGCGTGAGCGTGATCACACCCGGA |
| | ACCAATACAAGCAACCAGGTTGCCGTCCTGTATCAGGGAG |
| | TCAATTGCACAGAAGTGCCAGTTGCTATCCACGCAGACCAG |
| | CTGACTCCCACATGGCGGGTGTATAGCACCGGATCCAACGT |
| | GTTTCAGACCCGCGCCGGATGTCTCATTGGGGCCGAGCACG |
| | TGAATAACAGCTACGAGTGCGACATCCCCATTGGCGCCGG |
| | CATTTGTGCGTCTTACCAGACTCAGACCAACTCTCATGGCT |
| | CCGCCTCTTCCGTTGCTAGTCAGTCTATTATTGCCTATACCA |
| | TGAGCCTCGGAGCTGAGAATAGCGTGGCCTACTCCAATAAT |
| | TCCATCGCAATCCCTATAAACTTCACTATTTCTGTGACCAC |
| | CGAGATCCTGCCTGTGTCTATGACTAAGACTAGCGTTGATT |
| | GTACCATGTATATTTGTGGCGACTCTACCGAATGTTCTAAC |
| | CTGCTGCTTCAGTACGGCTCATTTTGCACACAGCTGAACAG |
| | AGCCCTGACTGGGATCGCTGTGGAGCAGGACAAGAACACA |
| | CAGGAGGTGTTTGCACAGGTGAAGCAGATCTATAAGACCC |
| | CTCCTATTAAGGATTTCGGCGGATTCAATTTCTCACAGATT |
| | CTGCCAGACCCCAGTAAGCCTTCCAAGAGGAGCTTCATCGA |
| | GGATCTCCTGTTTAACAAGGTGACCCTGGCAGACGCCGGCT |
| | TTATTAAGCAATATGGGGATTGCCTGGGCGACATTGCTGCC |
| | AGAGACCTGATTTGCGCCCAGAAATTCAATGGCCTCACAGT |
| | GCTGCCACCTCTGCTGACCGACGAGATGATCGCTCAATACA |
| | CTAGCGCACTGCTGGCCGGAACCATCACATCAGGCTGGAC |
| | CTTCGGGGCCGGAGCAGCACTGCAGATTCCATTCGCCATGC |
| | AGATGGCCTATAGATTCAACGGCATTGGCGTCACACAGAA |
| | CGTGCTGTACGAAAACCAGAAGCTCATCGCTAACCAGTTTA |
| | ATTCCGCAATTGGAAAGATCCAAGATTCACTCAGCTCAACC |
| | GCCTCTGCACTCGGAAAGCTGCAGGACGTGGTCAACCAGA |
| | ATGCTCAGGCCCTGAACACACTCGTCAAGCAGCTGTCCTCT |
| | AACTTTGGCGCTATCAGCTCCGTTCTGAACGACATTCTGGC |
| | ACGCCTGGATCCCCCAGAGGCTGAAGTCCAGATTGACCGC |
| | CTGATTACCGGCCGGCTGCAGTCTCTGCAAACATACGTGAC |
| | CCAGCAGCTGATCAGAGCAGCCGAGATCCGGGCATCCGCA |
| | AATCTGGCAGCAACTAAGATGAGCGAATGCGTGCTGGGCC |
| | AGTCCAAGCGGGTGGACTTTTGTGGCAAGGGCTACCACCTG |
| | ATGAGCTTCCCCCAGAGCGCCCCACATGGCGTTGTTTTTCT |
| | GCACGTGACCTATGTCCCTGCTCAGGAAAAGAACTTTACAA |
| | CTGCTCCTGCTATCTGCCATGACGGCAAGGCCCACTTCCCA |
| | CGGGAGGGAGTGTTTGTGTCCAATGGCACACACTGGTTCGT |
| | GACCCAGAGGAACTTCTATGAACCCCAGATCATCACCACTC |
| | ACAATACCTTCGTGTCTGGAAATTGCGACGTCGTGATCGGC |
| | ATCGTTAACAACACCGTGTACGACCCTCTCCAGCCAGAGCT |
| | GGACTCCTTTAAGGAGGAACTGGATAAGTATTTTAAGAACC |
| | ACACAAGCCCAGATGTGGATCTCGGGGACATCTCCGGAAT |
| | TAACGCCTCCGTGGTGAATATCCAGAAGGAGATTGACCGC |
| | CTAAATGAAGTTGCCAAGAACCTCAATGAGTCTCTGATTGA |
| | TCTGCAGGAACTGGGCAAGTATGAGCAGTATATCAAATGG |
| | CCCTGGTACATTTGGCTGGGGTTTATCGCCGGACTGATTGC |
| | CATCGTCATGGTGACCATCATGCTGTGTTGCATGACCTCCT |
| | GTTGTTCCTGTCTGAAGGGCTGCTGTAGTTGCGGCTCTTGCT |
| | GTAAATTCGACGAAGATGATAGCGAGCCCGTGCTGAAGGG |
| | CGTGAAGCTGCATTATACCTGA |
| SARS-CoV-2 S protein | (SEQ ID NO: 157) |
| mutated to remove a | MFVFLVLLPLVSSQCVNLTTRTQLPPAYTNSFTRGVYYPDKVF |
| furin cleavage site and | RSSVLHSTQDLFLPFFSNVTWFHAISGTNGTKRFDNPVLPFND |
| to replace residues 986 | GVYFASTEKSNIIRGWIFGTTLDSKTQSLLIVNNATNVVIKVCE |
| and 987 with proline | FQFCNDPFLGVYHKNNKSWMESEFRVYSSANNCTFEYVSQPF |
| and which contains the | LMDLEGKQGNFKNLREFVFKNIDGYFKIYSKHTPINLVRDLPQ |
| H69-, V70-, Y144-, | GFSALEPLVDLPIGINITRFQTLLALHRSYLTPGDSSSGWTAGA |
| N501Y, A570D, D614G, | AAYYVGYLQPRTFLLKYNENGTITDAVDCALDPLSETKCTLK |
| P681H, T716I, S982A | SFTVEKGIYQTSNFRVQPTESIVRFPNITNLCPFGEVFNATRFAS |
| and D1118H mutations | VYAWNRKRISNCVADYSVLYNSASFSTFKCYGVSPTKLNDLC |
| | FTNVYADSFVIRGDEVRQIAPGQTGKIADYNYKLPDDFTGCVI |
| | AWNSNNLDSKVGGNYNYLYRLFRKSNLKPFERDISTEIYQAG |
| | STPCNGVEGFNCYFPLQSYGFQPTYGVGYQPYRVVVLSFELL |
| | HAPATVCGPKKSTNLVKNKCVNFNFNGLTGTGVLTESNKKFL |
| | PFQQFGRDIDDTTDAVRDPQTLEILDITPCSFGGVSVITPGTNTS |
| | NQVAVLYQGVNCTEVPVAIHADQLTPTWRVYSTGSNVFQTR |
| | AGCLIGAEHVNNSYECDIPIGAGICASYQTQTNSHGSASSVAS |
| | QSIIAYTMSLGAENSVAYSNNSIAIPINFTISVTTEILPVSMTKTS |
| | VDCTMYICGDSTECSNLLLQYGSFCTQLNRALTGIAVEQDKN |
| | TQEVFAQVKQIYKTPPIKDFGGFNFSQILPDPSKPSKRSFIEDLL |
| | FNKVTLADAGFIKQYGDCLGDIAARDLICAQKFNGLTVLPPLL |
| | TDEMIAQYTSALLAGTITSGWTFGAGAALQIPFAMQMAYRFN |
| | GIGVTQNVLYENQKLIANQFNSAIGKIQDSLSSTASALGKLQD |
| | VVNQNAQALNTLVKQLSSNFGAISSVLNDILARLDPPEAEVQI |
| | DRLITGRLQSLQTYVTQQLIRAAEIRASANLAATKMSECVLGQ |
| | SKRVDFCGKGYHLMSFPQSAPHGVVFLHVTYVPAQEKNFTTA |
| | PAICHDGKAHFPREGVFVSNGTHWFVTQRNFYEPQIITTHNTF |
| | VSGNCDVVIGIVNNTVYDPLQPELDSFKEELDKYFKNHTSPDV |
| | DLGDISGINASVVNIQKEIDRLNEVAKNLNESLIDLQELGKYEQ |
| | YIKWPWYIWLGFIAGLIAIVMVTIMLCCMTSCCSCLKGCCSCG |
| | SCCKFDEDDSEPVLKGVKLHYT |
| Optimized nucleotide | (SEQ ID NO: 158) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| mutated to remove a | CACCAACTCCTTCACCAGAGGCGTGTATTACCCAGACAAGG |
| furin cleavage site and | TGTTTAGAAGCAGCGTGCTGCACTCTACCCAGGACCTCTTT |
| to replace residues 817, | CTGCCCTTTTTCAGCAACGTGACATGGTTTCACGCAATTTCC |
| 892, 899 and 942, 986 | GGCACTAATGGCACAAAGCGGTTCGACAATCCAGTCCTGC |
| and 987 with proline | CTTTCAACGATGGCGTCTACTTTGCATCTACTGAGAAATCC |
| and which contains the | AATATCATTAGGGGATGGATCTTCGGCACAACCCTGGATTC |
| H69-, V70-, Y144-, | TAAGACCCAGAGCCTGCTGATCGTCAACAACGCCACAAAC |
| N501Y, A570D, D614G, | GTGGTCATTAAGGTTTGCGAGTTTCAGTTCTGTAACGATCC |
| P681H, T716I, S982A | TTTTCTGGGCGTTTACCATAAGAACAATAAGAGCTGGATGG |
| and D1118H mutations | AGTCCGAGTTTAGAGTGTATAGCTCTGCAAATAATTGTACC |
| | TTTGAGTACGTGAGCCAGCCCTTTCTGATGGACCTGGAGGG |
| | AAAACAAGGAAACTTCAAAAACCTGCGGGAATTCGTTTTC |
| | AAAAACATCGACGGCTATTTCAAGATCTATAGCAAGCATA |
| | CCCCAATCAACCTCGTGAGGGACCTCCCCCAGGGCTTTAGC |
| | GCACTGGAGCCACTGGTTGACCTGCCTATCGGCATTAATAT |
| | CACAAGATTTCAGACCCTGCTGGCACTGCATAGAAGCTATC |
| | TGACCCCTGGAGACTCCTCTAGTGGGTGGACTGCCGGCGCC |
| | GCTGCCTACTATGTGGGCTATCTGCAGCCACGGACATTCCT |
| | GCTGAAATACAATGAGAACGGGACAATCACAGATGCTGTT |
| | GATTGCGCACTCGACCCCCTGTCCGAGACAAAGTGCACTCT |
| | CAAGAGCTTTACCGTCGAGAAGGGCATCTATCAGACCTCA |
| | AACTTCAGGGTGCAGCCCACAGAATCTATCGTGCGCTTCCC |
| | TAATATCACTAACCTGTGTCCTTTCGGTGAAGTGTTCAACG |
| | CCACCAGGTTTGCTAGCGTGTATGCCTGGAACAGGAAGAG |
| | GATCTCTAACTGCGTCGCCGACTATTCCGTGCTGTATAACA |
| | GCGCCTCCTTCTCCACATTCAAATGCTATGGAGTGAGCCCG |
| | ACAAAACTGAACGATCTCTGCTTTACAAATGTCTACGCCGA |
| | CTCTTTTGTGATCAGAGGGGACGAGGTCCGGCAGATCGCAC |
| | CAGGACAGACAGGCAAGATTGCTGACTACAACTATAAGCT |
| | GCCTGACGACTTCACAGGATGTGTGATCGCATGGAACTCAA |
| | ACAATCTGGACTCCAAAGTCGGGGGCAACTATAATTACCTG |
| | TATCGCCTGTTCCGGAAGTCCAACCTGAAGCCCTTCGAGAG |
| | GGACATCAGTACAGAGATCTATCAGGCTGGCTCCACCCCTT |
| | GCAATGGCGTCGAAGGCTTTAATTGTTATTTTCCCCTGCAG |
| | TCTTACGGGTTTCAGCCTACTTATGGAGTTGGGTACCAGCC |
| | ATACAGAGTGGTCGTGCTCAGCTTCGAGCTCCTGCATGCTC |
| | CAGCTACAGTTTGCGGGCCAAAGAAGTCCACTAACCTGGT |
| | GAAGAATAAGTGCGTCAACTTCAACTTTAACGGGCTCACCG |
| | GCACCGGCGTGCTGACTGAGAGCAACAAGAAGTTTCTGCC |
| | ATTTCAACAGTTTGGACGGGACATTGATGACACCACCGAT |
| | GCCGTTCGGGATCCACAGACCCTGGAAATTCTGGACATTAC |
| | ACCGTGCAGCTTCGGGGGCGTGAGCGTGATCACACCCGGA |
| | ACCAATACAAGCAACCAGGTTGCCGTCCTGTATCAGGGCG |
| | TCAATTGCACAGAAGTGCCAGTTGCTATCCACGCAGACCAG |
| | CTGACTCCCACATGGCGGGTGTATAGCACCGGATCCAACGT |
| | GTTTCAGACCCGCGCCGGATGTCTCATTGGGGCCGAGCACG |
| | TGAATAACAGCTACGAGTGCGACATCCCCATTGGCGCCGG |
| | CATTTGTGCGTCTTACCAGACTCAGACCAACTCTCATGGCT |
| | CCGCCTCTTCCGTTGCTAGTCAGTCTATTATTGCCTATACCA |
| | TGAGCCTCGGAGCTGAGAATAGCGTGGCCTACTCCAATAAT |
| | TCCATCGCAATCCCTATAAACTTCACTATTTCTGTGACCAC |
| | CGAGATCCTGCCTGTGTCTATGACTAAGACTAGCGTTGATT |
| | GTACCATGTATATTTGTGGCGACTCTACCGAATGTTCTAAC |
| | CTGCTGCTTCAGTACGGCTCATTTTGCACACAGCTGAACAG |
| | AGCCCTGACTGGGATCGCTGTGGAGCAGGACAAGAACACA |
| | CAGGAGGTGTTTGCACAGGTGAAGCAGATCTATAAGACCC |
| | CTCCTATTAAGGATTTCGGCGGATTCAATTTCTCACAGATT |
| | CTGCCAGACCCCAGTAAGCCTTCCAAGAGGAGCCCTATCG |
| | AGGATCTCCTGTTTAACAAGGTGACCCTGGCAGACGCCGGC |
| | TTTATTAAGCAATATGGGGATTGCCTGGGCGACATTGCTGC |
| | CAGAGACCTGATTTGCGCCCAGAAATTCAATGGCCTCACAG |
| | TGCTGCCACCTCTGCTGACCGACGAGATGATCGCTCAATAC |
| | ACTAGCGCACTGCTGGCCGGAACCATCACATCAGGCTGGA |
| | CCTTCGGGGCCGGACCAGCACTGCAGATTCCATTCCCTATG |
| | CAGATGGCCTATAGATTCAACGGCATTGGCGTCACACAGA |
| | ACGTGCTGTACGAAAACCAGAAGCTCATCGCTAACCAGTTT |
| | AATTCCGCAATTGGAAAGATCCAAGATTCACTCAGCTCAAC |
| | CCCCTCTGCACTCGGAAAGCTGCAGGACGTGGTCAACCAG |
| | AATGCTCAGGCCCTGAACACACTCGTCAAGCAGCTGTCCTC |
| | TAACTTTGGCGCTATCAGCTCCGTTCTGAACGACATTCTGG |
| | CACGCCTGGATCCCCCAGAGGCTGAAGTCCAGATTGACCG |
| | CCTGATTACCGGCCGGCTGCAGTCTCTGCAAACATACGTGA |
| | CCCAGCAGCTGATCAGAGCAGCCGAGATCCGGGCATCCGC |
| | AAATCTGGCAGCAACTAAGATGAGCGAATGCGTGCTGGGC |
| | CAGTCCAAGCGGGTGGACTTTTGTGGCAAGGGCTACCACCT |
| | GATGAGCTTCCCCCAGAGCGCCCCACATGGCGTTGTTTTTC |
| | TGCACGTGACCTATGTCCCTGCTCAGGAAAAGAACTTTACA |
| | ACTGCTCCTGCTATCTGCCATGACGGCAAGGCCCACTTCCC |
| | ACGGGAGGGAGTGTTTGTGTCCAATGGCACACACTGGTTCG |
| | TGACCCAGAGGAACTTCTATGAACCCCAGATCATCACCACT |
| | CACAATACCTTCGTGTCTGGAAATTGCGACGTCGTGATCGG |
| | CATCGTTAACAACACCGTGTACGACCCTCTCCAGCCAGAGC |
| | TGGACTCCTTTAAGGAGGAACTGGATAAGTATTTTAAGAAC |
| | CACACAAGCCCAGATGTGGATCTCGGGGACATCTCCGGAA |
| | TTAACGCCTCCGTGGTGAATATCCAGAAGGAGATTGACCGC |
| | CTAAATGAAGTTGCCAAGAACCTCAATGAGTCTCTGATTGA |
| | TCTGCAGGAACTGGGCAAGTATGAGCAGTATATCAAATGG |
| | CCCTGGTACATTTGGCTGGGGTTTATCGCCGGACTGATTGC |
| | CATCGTCATGGTGACCATCATGCTGTGTTGCATGACCTCCT |
| | GTTGTTCCTGTCTGAAGGGCTGCTGTAGTTGCGGCTCTTGCT |
| | GTAAATTCGACGAAGATGATAGCGAGCCCGTGCTGAAGGG |
| | CGTGAAGCTGCATTATACCTGA |
| SARS-CoV-2 S protein | (SEQ ID NO: 159) |
| mutated to remove a | MFVFLVLLPLVSSQCVNLTTRTQLPPAYTNSFTRGVYYPDKVF |
| furin cleavage site and | RSSVLHSTQDLFLPFFSNVTWFHAISGTNGTKRFDNPVLPFND |
| to replace residues 817, | GVYFASTEKSNIIRGWIFGTTLDSKTQSLLIVNNATNVVIKVCE |
| 892, 899 and 942, 986 | FQFCNDPFLGVYHKNNKSWMESEFRVYSSANNCTFEYVSQPF |
| and 987 with proline | LMDLEGKQGNFKNLREFVFKNIDGYFKIYSKHTPINLVRDLPQ |
| and which contains the | GFSALEPLVDLPIGINITRFQTLLALHRSYLTPGDSSSGWTAGA |
| H69-, V70-, Y144-, | AAYYVGYLQPRTFLLKYNENGTITDAVDCALDPLSETKCTLK |
| N501Y, A570D, D614G, | SFTVEKGIYQTSNFRVQPTESIVRFPNITNLCPFGEVENATRFAS |
| P681H, T716I, S982A | VYAWNRKRISNCVADYSVLYNSASFSTFKCYGVSPTKLNDLC |
| and D1118H mutations | FTNVYADSFVIRGDEVRQIAPGQTGKIADYNYKLPDDFTGCVI |
| | AWNSNNLDSKVGGNYNYLYRLFRKSNLKPFERDISTEIYQAG |
| | STPCNGVEGFNCYFPLQSYGFQPTYGVGYQPYRVVVLSFELL |
| | HAPATVCGPKKSTNLVKNKCVNFNFNGLTGTGVLTESNKKFL |
| | PFQQFGRDIDDTTDAVRDPQTLEILDITPCSFGGVSVITPGTNTS |
| | NQVAVLYQGVNCTEVPVAIHADQLTPTWRVYSTGSNVFQTR |
| | AGCLIGAEHVNNSYECDIPIGAGICASYQTQTNSHGSASSVAS |
| | QSIIAYTMSLGAENSVAYSNNSIAIPINFTISVTTEILPVSMTKTS |
| | VDCTMYICGDSTECSNLLLQYGSFCTQLNRALTGIAVEQDKN |
| | TQEVFAQVKQIYKTPPIKDFGGFNFSQILPDPSKPSKRSPIEDLL |
| | FNKVTLADAGFIKQYGDCLGDIAARDLICAQKFNGLTVLPPLL |
| | TDEMIAQYTSALLAGTITSGWTFGAGPALQIPFPMQMAYRFN |
| | GIGVTQNVLYENQKLIANQFNSAIGKIQDSLSSTPSALGKLQD |
| | VVNQNAQALNTLVKQLSSNFGAISSVLNDILARLDPPEAEVQI |
| | DRLITGRLQSLQTYVTQQLIRAAEIRASANLAATKMSECVLGQ |
| | SKRVDFCGKGYHLMSFPQSAPHGVVFLHVTYVPAQEKNFTTA |
| | PAICHDGKAHFPREGVFVSNGTHWFVTQRNFYEPQIITTHNTF |
| | VSGNCDVVIGIVNNTVYDPLQPELDSFKEELDKYFKNHTSPDV |
| | DLGDISGINASVVNIQKEIDRLNEVAKNLNESLIDLQELGKYEQ |
| | YIKWPWYIWLGFIAGLIAIVMVTIMLCCMTSCCSCLKGCCSCG |
| | SCCKFDEDDSEPVLKGVKLHYT |
| Optimized nucleotide | (SEQ ID NO: 160) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| containing the D80A, | CACCAACTCCTTCACCAGAGGCGTGTATTACCCAGACAAGG |
| D215G, L242-, A243-, | TGTTTAGAAGCAGCGTGCTGCACTCTACCCAGGACCTCTTT |
| L244-, K417N, E484K, | CTGCCCTTTTTCAGCAACGTGACATGGTTTCACGCAATTCA |
| N501Y, D614G and | CGTGTCCGGCACTAATGGCACAAAGCGGTTCGCCAATCCA |
| A701V mutations | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGGCCTCCCCCAG |
| | GGCTTTAGCGCACTGGAGCCACTGGTTGACCTGCCTATCGG |
| | CATTAATATCACAAGATTTCAGACCCTGCATAGAAGCTATC |
| | TGACCCCTGGAGACTCCTCTAGTGGGTGGACTGCCGGCGCC |
| | GCTGCCTACTATGTGGGCTATCTGCAGCCACGGACATTCCT |
| | GCTGAAATACAATGAGAACGGGACAATCACAGATGCTGTT |
| | GATTGCGCACTCGACCCCCTGTCCGAGACAAAGTGCACTCT |
| | CAAGAGCTTTACCGTCGAGAAGGGCATCTATCAGACCTCA |
| | AACTTCAGGGTGCAGCCCACAGAATCTATCGTGCGCTTCCC |
| | TAATATCACTAACCTGTGTCCTTTCGGTGAAGTGTTCAACG |
| | CCACCAGGTTTGCTAGCGTGTATGCCTGGAACAGGAAGAG |
| | GATCTCTAACTGCGTCGCCGACTATTCCGTGCTGTATAACA |
| | GCGCCTCCTTCTCCACATTCAAATGCTATGGAGTGAGCCCG |
| | ACAAAACTGAACGATCTCTGCTTTACAAATGTCTACGCCGA |
| | CTCTTTTGTGATCAGAGGGGACGAGGTCCGGCAGATCGCAC |
| | CAGGACAGACAGGCAACATTGCTGACTACAACTATAAGCT |
| | GCCTGACGACTTCACAGGATGTGTGATCGCATGGAACTCAA |
| | ACAATCTGGACTCCAAAGTCGGGGGCAACTATAATTACCTG |
| | TATCGCCTGTTCCGGAAGTCCAACCTGAAGCCCTTCGAGAG |
| | GGACATCAGTACAGAGATCTATCAGGCTGGCTCCACCCCTT |
| | GCAATGGCGTCAAGGGCTTTAATTGTTATTTTCCCCTGCAG |
| | TCTTACGGGTTTCAGCCTACTTACGGAGTTGGGTACCAGCC |
| | ATACAGAGTGGTCGTGCTCAGCTTCGAGCTCCTGCATGCTC |
| | CAGCTACAGTTTGCGGGCCAAAGAAGTCCACTAACCTGGT |
| | GAAGAATAAGTGCGTCAACTTCAACTTTAACGGGCTCACCG |
| | GCACCGGCGTGCTGACTGAGAGCAACAAGAAGTTTCTGCC |
| | ATTTCAACAGTTTGGACGGGACATTGCCGACACCACCGATG |
| | CCGTTCGGGATCCACAGACCCTGGAAATTCTGGACATTACA |
| | CCGTGCAGCTTCGGGGGCGTGAGCGTGATCACACCCGGAA |
| | CCAATACAAGCAACCAGGTTGCCGTCCTGTATCAGGGCGT |
| | CAATTGCACAGAAGTGCCAGTTGCTATCCACGCAGACCAG |
| | CTGACTCCCACATGGCGGGTGTATAGCACCGGATCCAACGT |
| | GTTTCAGACCCGCGCCGGATGTCTCATTGGGGCCGAGCACG |
| | TGAATAACAGCTACGAGTGCGACATCCCCATTGGCGCCGG |
| | CATTTGTGCGTCTTACCAGACTCAGACCAACTCTCCTAGAA |
| | GGGCCAGGTCCGTTGCTAGTCAGTCTATTATTGCCTATACC |
| | ATGAGCCTCGGAGTGGAGAATAGCGTGGCCTACTCCAATA |
| | ATTCCATCGCAATCCCTACTAACTTCACTATTTCTGTGACCA |
| | CCGAGATCCTGCCTGTGTCTATGACTAAGACTAGCGTTGAT |
| | TGTACCATGTATATTTGTGGCGACTCTACCGAATGTTCTAA |
| | CCTGCTGCTTCAGTACGGCTCATTTTGCACACAGCTGAACA |
| | GAGCCCTGACTGGGATCGCTGTGGAGCAGGACAAGAACAC |
| | ACAGGAGGTGTTTGCACAGGTGAAGCAGATCTATAAGACC |
| | CCTCCTATTAAGGATTTCGGCGGATTCAATTTCTCACAGAT |
| | TCTGCCAGACCCCAGTAAGCCTTCCAAGAGGAGCTTCATCG |
| | AGGATCTCCTGTTTAACAAGGTGACCCTGGCAGACGCCGGC |
| | TTTATTAAGCAATATGGGGATTGCCTGGGCGACATTGCTGC |
| | CAGAGACCTGATTTGCGCCCAGAAATTCAATGGCCTCACAG |
| | TGCTGCCACCTCTGCTGACCGACGAGATGATCGCTCAATAC |
| | ACTAGCGCACTGCTGGCCGGAACCATCACATCAGGCTGGA |
| | CCTTCGGGGCCGGAGCAGCACTGCAGATTCCATTCGCCATG |
| | CAGATGGCCTATAGATTCAACGGCATTGGCGTCACACAGA |
| | ACGTGCTGTACGAAAACCAGAAGCTCATCGCTAACCAGTTT |
| | AATTCCGCAATTGGAAAGATCCAAGATTCACTCAGCTCAAC |
| | CGCCTCTGCACTCGGAAAGCTGCAGGACGTGGTCAACCAG |
| | AATGCTCAGGCCCTGAACACACTCGTCAAGCAGCTGTCCTC |
| | TAACTTTGGCGCTATCAGCTCCGTTCTGAACGACATTCTGA |
| | GCCGCCTGGATAAGGTGGAGGCTGAAGTCCAGATTGACCG |
| | CCTGATTACCGGCCGGCTGCAGTCTCTGCAAACATACGTGA |
| | CCCAGCAGCTGATCAGAGCAGCCGAGATCCGGGCATCCGC |
| | AAATCTGGCAGCAACTAAGATGAGCGAATGCGTGCTGGGC |
| | CAGTCCAAGCGGGTGGACTTTTGTGGCAAGGGCTACCACCT |
| | GATGAGCTTCCCCCAGAGCGCCCCACATGGCGTTGTTTTTC |
| | TGCACGTGACCTATGTCCCTGCTCAGGAAAAGAACTTTACA |
| | ACTGCTCCTGCTATCTGCCATGACGGCAAGGCCCACTTCCC |
| | ACGGGAGGGAGTGTTTGTGTCCAATGGCACACACTGGTTCG |
| | TGACCCAGAGGAACTTCTATGAACCCCAGATCATCACCACT |
| | GACAATACCTTCGTGTCTGGAAATTGCGACGTCGTGATCGG |
| | CATCGTTAACAACACCGTGTACGACCCTCTCCAGCCAGAGC |
| | TGGACTCCTTTAAGGAGGAACTGGATAAGTATTTTAAGAAC |
| | CACACAAGCCCAGATGTGGATCTCGGGGACATCTCCGGAA |
| | TTAACGCCTCCGTGGTGAATATCCAGAAGGAGATTGACCGC |
| | CTAAATGAAGTTGCCAAGAACCTCAATGAGTCTCTGATTGA |
| | TCTGCAGGAACTGGGCAAGTATGAGCAGTATATCAAATGG |
| | CCCTGGTACATTTGGCTGGGGTTTATCGCCGGACTGATTGC |
| | CATCGTCATGGTGACCATCATGCTGTGTTGCATGACCTCCT |
| | GTTGTTCCTGTCTGAAGGGCTGCTGTAGTTGCGGCTCTTGCT |
| | GTAAATTCGACGAAGATGATAGCGAGCCCGTGCTGAAGGG |
| | CGTGAAGCTGCATTATACCTGA |
| SARS-CoV-2 S protein | (SEQ ID NO: 161) |
| containing the D80A, | MFVFLVLLPLVSSQCVNLTTRTQLPPAYTNSFTRGVYYPDKVF |
| D215G, L242-, A243-, | RSSVLHSTQDLFLPFFSNVTWFHAIHVSGTNGTKRFANPVLPF |
| L244-, K417N, E484K, | NDGVYFASTEKSNIIRGWIFGTTLDSKTQSLLIVNNATNVVIKV |
| N501Y, D614G and | CEFQFCNDPFLGVYYHKNNKSWMESEFRVYSSANNCTFEYVS |
| A701V mutations | QPFLMDLEGKQGNFKNLREFVFKNIDGYFKIYSKHTPINLVRG |
| | LPQGFSALEPLVDLPIGINITRFQTLHRSYLTPGDSSSGWTAGA |
| | AAYYVGYLQPRTFLLKYNENGTITDAVDCALDPLSETKCTLK |
| | SFTVEKGIYQTSNFRVQPTESIVRFPNITNLCPFGEVFNATRFAS |
| | VYAWNRKRISNCVADYSVLYNSASFSTFKCYGVSPTKLNDLC |
| | FTNVYADSFVIRGDEVRQIAPGQTGNIADYNYKLPDDFTGCVI |
| | AWNSNNLDSKVGGNYNYLYRLFRKSNLKPFERDISTEIYQAG |
| | STPCNGVKGFNCYFPLQSYGFQPTYGVGYQPYRVVVLSFELL |
| | HAPATVCGPKKSTNLVKNKCVNFNFNGLTGTGVLTESNKKFL |
| | PFQQFGRDIADTTDAVRDPQTLEILDITPCSFGGVSVITPGTNTS |
| | NQVAVLYQGVNCTEVPVAIHADQLTPTWRVYSTGSNVFQTR |
| | AGCLIGAEHVNNSYECDIPIGAGICASYQTQTNSPRRARSVAS |
| | QSIIAYTMSLGVENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDKVEAEV |
| | QIDRLITGRLQSLQTYVTQQLIRAAEIRASANLAATKMSECVL |
| | GQSKRVDFCGKGYHLMSFPQSAPHGVVFLHVTYVPAQEKNF |
| | TTAPAICHDGKAHFPREGVFVSNGTHWFVTQRNFYEPQIITTD |
| | NTFVSGNCDVVIGIVNNTVYDPLQPELDSFKEELDKYFKNHTS |
| | PDVDLGDISGINASVVNIQKEIDRLNEVAKNLNESLIDLQELGK |
| | YEQYIKWPWYIWLGFIAGLIAIVMVTIMLCCMTSCCSCLKGCC |
| | SCGSCCKFDEDDSEPVLKGVKLHYT |
| Optimized nucleotide | (SEQ ID NO: 162) |
| sequence encoding a | ATGTTCGTCTTCCTCGTGCTGCTCCCACTCGTTTCTTCCCAG |
| SARS-CoV-2 S protein | TGTGTCAACCTGACAACTAGGACTCAGCTGCCACCAGCCTA |
| containing mutated to | CACCAACTCCTTCACCAGAGGCGTGTATTACCCAGACAAGG |
| remove a furin cleavage | TGTTTAGAAGCAGCGTGCTGCACTCTACCCAGGACCTCTTT |
| site and to replace | CTGCCCTTTTTCAGCAACGTGACATGGTTTCACGCAATTCA |
| residues 986 and 987 | CGTGTCCGGCACTAATGGCACAAAGCGGTTCGCCAATCCA |
| with proline and which | GTCCTGCCTTTCAACGATGGCGTCTACTTTGCATCTACTGA |
| contains the D80A, | GAAATCCAATATCATTAGGGGATGGATCTTCGGCACAACCC |
| D215G, L242-, A243-, | TGGATTCTAAGACCCAGAGCCTGCTGATCGTCAACAACGCC |
| L244-, K417N, E484K, | ACAAACGTGGTCATTAAGGTTTGCGAGTTTCAGTTCTGTAA |
| N501Y, D614G and | CGATCCTTTTCTGGGCGTGTATTATCATAAGAACAATAAGA |
| A701V mutations | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGGCCTCCCCCAG |
| | GGCTTTAGCGCACTGGAGCCACTGGTTGACCTGCCTATCGG |
| | CATTAATATCACAAGATTTCAGACCCTGCATAGAAGCTATC |
| | TGACCCCTGGAGACTCCTCTAGTGGGTGGACTGCCGGCGCC |
| | GCTGCCTACTATGTGGGCTATCTGCAGCCACGGACATTCCT |
| | GCTGAAATACAATGAGAACGGGACAATCACAGATGCTGTT |
| | GATTGCGCACTCGACCCCCTGTCCGAGACAAAGTGCACTCT |
| | CAAGAGCTTTACCGTCGAGAAGGGCATCTATCAGACCTCA |
| | AACTTCAGGGTGCAGCCCACAGAATCTATCGTGCGCTTCCC |
| | TAATATCACTAACCTGTGTCCTTTCGGTGAAGTGTTCAACG |
| | CCACCAGGTTTGCTAGCGTGTATGCCTGGAACAGGAAGAG |
| | GATCTCTAACTGCGTCGCCGACTATTCCGTGCTGTATAACA |
| | GCGCCTCCTTCTCCACATTCAAATGCTATGGAGTGAGCCCG |
| | ACAAAACTGAACGATCTCTGCTTTACAAATGTCTACGCCGA |
| | CTCTTTTGTGATCAGAGGGGACGAGGTCCGGCAGATCGCAC |
| | CAGGACAGACAGGCAACATTGCTGACTACAACTATAAGCT |
| | GCCTGACGACTTCACAGGATGTGTGATCGCATGGAACTCAA |
| | ACAATCTGGACTCCAAAGTCGGGGGCAACTATAATTACCTG |
| | TATCGCCTGTTCCGGAAGTCCAACCTGAAGCCCTTCGAGAG |
| | GGACATCAGTACAGAGATCTATCAGGCTGGCTCCACCCCTT |
| | GCAATGGCGTCAAGGGCTTTAATTGTTATTTTCCCCTGCAG |
| | TCTTACGGGTTTCAGCCTACTTACGGAGTTGGGTACCAGCC |
| | ATACAGAGTGGTCGTGCTCAGCTTCGAGCTCCTGCATGCTC |
| | CAGCTACAGTTTGCGGGCCAAAGAAGTCCACTAACCTGGT |
| | GAAGAATAAGTGCGTCAACTTCAACTTTAACGGGCTCACCG |
| | GCACCGGCGTGCTGACTGAGAGCAACAAGAAGTTTCTGCC |
| | ATTTCAACAGTTTGGACGGGACATTGCCGACACCACCGATG |
| | CCGTTCGGGATCCACAGACCCTGGAAATTCTGGACATTACA |
| | CCGTGCAGCTTCGGGGGCGTGAGCGTGATCACACCCGGAA |
| | CCAATACAAGCAACCAGGTTGCCGTCCTGTATCAGGGCGT |
| | CAATTGCACAGAAGTGCCAGTTGCTATCCACGCAGACCAG |
| | CTGACTCCCACATGGCGGGTGTATAGCACCGGATCCAACGT |
| | GTTTCAGACCCGCGCCGGATGTCTCATTGGGGCCGAGCACG |
| | TGAATAACAGCTACGAGTGCGACATCCCCATTGGCGCCGG |
| | CATTTGTGCGTCTTACCAGACTCAGACCAACTCTCCTGGCT |
| | CCGCCTCTTCCGTTGCTAGTCAGTCTATTATTGCCTATACCA |
| | TGAGCCTCGGAGTGGAGAATAGCGTGGCCTACTCCAATAA |
| | TTCCATCGCAATCCCTACTAACTTCACTATTTCTGTGACCAC |
| | CGAGATCCTGCCTGTGTCTATGACTAAGACTAGCGTTGATT |
| | GTACCATGTATATTTGTGGCGACTCTACCGAATGTTCTAAC |
| | CTGCTGCTTCAGTACGGCTCATTTTGCACACAGCTGAACAG |
| | AGCCCTGACTGGGATCGCTGTGGAGCAGGACAAGAACACA |
| | CAGGAGGTGTTTGCACAGGTGAAGCAGATCTATAAGACCC |
| | CTCCTATTAAGGATTTCGGCGGATTCAATTTCTCACAGATT |
| | CTGCCAGACCCCAGTAAGCCTTCCAAGAGGAGCTTCATCGA |
| | GGATCTCCTGTTTAACAAGGTGACCCTGGCAGACGCCGGCT |
| | TTATTAAGCAATATGGGGATTGCCTGGGCGACATTGCTGCC |
| | AGAGACCTGATTTGCGCCCAGAAATTCAATGGCCTCACAGT |
| | GCTGCCACCTCTGCTGACCGACGAGATGATCGCTCAATACA |
| | CTAGCGCACTGCTGGCCGGAACCATCACATCAGGCTGGAC |
| | CTTCGGGGCCGGAGCAGCACTGCAGATTCCATTCGCCATGC |
| | AGATGGCCTATAGATTCAACGGCATTGGCGTCACACAGAA |
| | CGTGCTGTACGAAAACCAGAAGCTCATCGCTAACCAGTTTA |
| | ATTCCGCAATTGGAAAGATCCAAGATTCACTCAGCTCAACC |
| | GCCTCTGCACTCGGAAAGCTGCAGGACGTGGTCAACCAGA |
| | ATGCTCAGGCCCTGAACACACTCGTCAAGCAGCTGTCCTCT |
| | AACTTTGGCGCTATCAGCTCCGTTCTGAACGACATTCTGAG |
| | CCGCCTGGATCCCCCAGAGGCTGAAGTCCAGATTGACCGCC |
| | TGATTACCGGCCGGCTGCAGTCTCTGCAAACATACGTGACC |
| | CAGCAGCTGATCAGAGCAGCCGAGATCCGGGCATCCGCAA |
| | ATCTGGCAGCAACTAAGATGAGCGAATGCGTGCTGGGCCA |
| | GTCCAAGCGGGTGGACTTTTGTGGCAAGGGCTACCACCTGA |
| | TGAGCTTCCCCCAGAGCGCCCCACATGGCGTTGTTTTTCTG |
| | CACGTGACCTATGTCCCTGCTCAGGAAAAGAACTTTACAAC |
| | TGCTCCTGCTATCTGCCATGACGGCAAGGCCCACTTCCCAC |
| | GGGAGGGAGTGTTTGTGTCCAATGGCACACACTGGTTCGTG |
| | ACCCAGAGGAACTTCTATGAACCCCAGATCATCACCACTGA |
| | CAATACCTTCGTGTCTGGAAATTGCGACGTCGTGATCGGCA |
| | TCGTTAACAACACCGTGTACGACCCTCTCCAGCCAGAGCTG |
| | GACTCCTTTAAGGAGGAACTGGATAAGTATTTTAAGAACCA |
| | CACAAGCCCAGATGTGGATCTCGGGGACATCTCCGGAATT |
| | AACGCCTCCGTGGTGAATATCCAGAAGGAGATTGACCGCC |
| | TAAATGAAGTTGCCAAGAACCTCAATGAGTCTCTGATTGAT |
| | CTGCAGGAACTGGGCAAGTATGAGCAGTATATCAAATGGC |
| | CCTGGTACATTTGGCTGGGGTTTATCGCCGGACTGATTGCC |
| | ATCGTCATGGTGACCATCATGCTGTGTTGCATGACCTCCTG |
| | TTGTTCCTGTCTGAAGGGCTGCTGTAGTTGCGGCTCTTGCTG |
| | TAAATTCGACGAAGATGATAGCGAGCCCGTGCTGAAGGGC |
| | GTGAAGCTGCATTATACCTGA |
| SARS-CoV-2 S protein | (SEQ ID NO: 163) |
| containing mutated to | MFVFLVLLPLVSSQCVNLTTRTQLPPAYTNSFTRGVYYPDKVF |
| remove a furin cleavage | RSSVLHSTQDLFLPFFSNVTWFHAIHVSGTNGTKRFANPVLPF |
| site and to replace | NDGVYFASTEKSNIIRGWIFGTTLDSKTQSLLIVNNATNVVIKV |
| residues 986 and 987 | CEFQFCNDPFLGVYYHKNNKSWMESEFRVYSSANNCTFEYVS |
| with proline and which | QPFLMDLEGKQGNFKNLREFVFKNIDGYFKIYSKHTPINLVRG |
| contains the D80A, | LPQGFSALEPLVDLPIGINITRFQTLHRSYLTPGDSSSGWTAGA |
| D215G, L242-, A243-, | AAYYVGYLQPRTFLLKYNENGTITDAVDCALDPLSETKCTLK |
| L244-, K417N, E484K, | SFTVEKGIYQTSNFRVQPTESIVRFPNITNLCPFGEVFNATRFAS |
| N501Y, D614G and | VYAWNRKRISNCVADYSVLYNSASFSTFKCYGVSPTKLNDLC |
| A701V mutations | FTNVYADSFVIRGDEVRQIAPGQTGNIADYNYKLPDDFTGCVI |
| | AWNSNNLDSKVGGNYNYLYRLFRKSNLKPFERDISTEIYQAG |
| | STPCNGVKGFNCYFPLQSYGFQPTYGVGYQPYRVVVLSFELL |
| | HAPATVCGPKKSTNLVKNKCVNFNFNGLTGTGVLTESNKKFL |
| | PFQQFGRDIADTTDAVRDPQTLEILDITPCSFGGVSVITPGTNTS |
| | NQVAVLYQGVNCTEVPVAIHADQLTPTWRVYSTGSNVFQTR |
| | AGCLIGAEHVNNSYECDIPIGAGICASYQTQTNSPGSASSVASQ |
| | SIIAYTMSLGVENSVAYSNNSIAIPTNFTISVTTEILPVSMTKTS |
| | VDCTMYICGDSTECSNLLLQYGSFCTQLNRALTGIAVEQDKN |
| | TQEVFAQVKQIYKTPPIKDFGGFNFSQILPDPSKPSKRSFIEDLL |
| | FNKVTLADAGFIKQYGDCLGDIAARDLICAQKFNGLTVLPPLL |
| | TDEMIAQYTSALLAGTITSGWTFGAGAALQIPFAMQMAYRFN |
| | GIGVTQNVLYENQKLIANQFNSAIGKIQDSLSSTASALGKLQD |
| | VVNQNAQALNTLVKQLSSNFGAISSVLNDILSRLDPPEAEVQI |
| | DRLITGRLQSLQTYVTQQLIRAAEIRASANLAATKMSECVLGQ |
| | SKRVDFCGKGYHLMSFPQSAPHGVVFLHVTYVPAQEKNFTTA |
| | PAICHDGKAHFPREGVFVSNGTHWFVTQRNFYEPQIITTDNTF |
| | VSGNCDVVIGIVNNTVYDPLQPELDSFKEELDKYFKNHTSPDV |
| | DLGDISGINASVVNIQKEIDRLNEVAKNLNESLIDLQELGKYEQ |
| | YIKWPWYIWLGFIAGLIAIVMVTIMLCCMTSCCSCLKGCCSCG |
| | SCCKFDEDDSEPVLKGVKLHYT |
| Optimized nucleotide | (SEQ ID NO: 164) |
| sequence encoding a | ATGTTCGTCTTCCTCGTGCTGCTCCCACTCGTTTCTTCCCAG |
| SARS-CoV-2 S protein | TGTGTCAACTTCACAACTAGGACTCAGCTGCCACCAGCCTA |
| containing the L18F, | CACCAACTCCTTCACCAGAGGCGTGTATTACCCAGACAAGG |
| D80A, D215G, L242-, | TGTTTAGAAGCAGCGTGCTGCACTCTACCCAGGACCTCTTT |
| A243-, L244-, K417N, | CTGCCCTTTTTCAGCAACGTGACATGGTTTCACGCAATTCA |
| E484K, N501Y, D614G | CGTGTCCGGCACTAATGGCACAAAGCGGTTCGCCAATCCA |
| and A701V mutations | GTCCTGCCTTTCAACGATGGCGTCTACTTTGCATCTACTGA |
| | GAAATCCAATATCATTAGGGGATGGATCTTCGGCACAACCC |
| | TGGATTCTAAGACCCAGAGCCTGCTGATCGTCAACAACGCC |
| | ACAAACGTGGTCATTAAGGTTTGCGAGTTTCAGTTCTGTAA |
| | CGATCCTTTTCTGGGCGTGTATTATCATAAGAACAATAAGA |
| | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGGCCTCCCCCAG |
| | GGCTTTAGCGCACTGGAGCCACTGGTTGACCTGCCTATCGG |
| | CATTAATATCACAAGATTTCAGACCCTGCATAGAAGCTATC |
| | TGACCCCTGGAGACTCCTCTAGTGGGTGGACTGCCGGCGCC |
| | GCTGCCTACTATGTGGGCTATCTGCAGCCACGGACATTCCT |
| | GCTGAAATACAATGAGAACGGGACAATCACAGATGCTGTT |
| | GATTGCGCACTCGACCCCCTGTCCGAGACAAAGTGCACTCT |
| | CAAGAGCTTTACCGTCGAGAAGGGCATCTATCAGACCTCA |
| | AACTTCAGGGTGCAGCCCACAGAATCTATCGTGCGCTTCCC |
| | TAATATCACTAACCTGTGTCCTTTCGGTGAAGTGTTCAACG |
| | CCACCAGGTTTGCTAGCGTGTATGCCTGGAACAGGAAGAG |
| | GATCTCTAACTGCGTCGCCGACTATTCCGTGCTGTATAACA |
| | GCGCCTCCTTCTCCACATTCAAATGCTATGGAGTGAGCCCG |
| | ACAAAACTGAACGATCTCTGCTTTACAAATGTCTACGCCGA |
| | CTCTTTTGTGATCAGAGGGGACGAGGTCCGGCAGATCGCAC |
| | CAGGACAGACAGGCAACATTGCTGACTACAACTATAAGCT |
| | GCCTGACGACTTCACAGGATGTGTGATCGCATGGAACTCAA |
| | ACAATCTGGACTCCAAAGTCGGGGGCAACTATAATTACCTG |
| | TATCGCCTGTTCCGGAAGTCCAACCTGAAGCCCTTCGAGAG |
| | GGACATCAGTACAGAGATCTATCAGGCTGGCTCCACCCCTT |
| | GCAATGGCGTCAAGGGCTTTAATTGTTATTTTCCCCTGCAG |
| | TCTTACGGGTTTCAGCCTACTTACGGAGTTGGGTACCAGCC |
| | ATACAGAGTGGTCGTGCTCAGCTTCGAGCTCCTGCATGCTC |
| | CAGCTACAGTTTGCGGGCCAAAGAAGTCCACTAACCTGGT |
| | GAAGAATAAGTGCGTCAACTTCAACTTTAACGGGCTCACCG |
| | GCACCGGCGTGCTGACTGAGAGCAACAAGAAGTTTCTGCC |
| | ATTTCAACAGTTTGGACGGGACATTGCCGACACCACCGATG |
| | CCGTTCGGGATCCACAGACCCTGGAAATTCTGGACATTACA |
| | CCGTGCAGCTTCGGGGGCGTGAGCGTGATCACACCCGGAA |
| | CCAATACAAGCAACCAGGTTGCCGTCCTGTATCAGGGCGT |
| | CAATTGCACAGAAGTGCCAGTTGCTATCCACGCAGACCAG |
| | CTGACTCCCACATGGCGGGTGTATAGCACCGGATCCAACGT |
| | GTTTCAGACCCGCGCCGGATGTCTCATTGGGGCCGAGCACG |
| | TGAATAACAGCTACGAGTGCGACATCCCCATTGGCGCCGG |
| | CATTTGTGCGTCTTACCAGACTCAGACCAACTCTCCTAGAA |
| | GGGCCAGGTCCGTTGCTAGTCAGTCTATTATTGCCTATACC |
| | ATGAGCCTCGGAGTGGAGAATAGCGTGGCCTACTCCAATA |
| | ATTCCATCGCAATCCCTACTAACTTCACTATTTCTGTGACCA |
| | CCGAGATCCTGCCTGTGTCTATGACTAAGACTAGCGTTGAT |
| | TGTACCATGTATATTTGTGGCGACTCTACCGAATGTTCTAA |
| | CCTGCTGCTTCAGTACGGCTCATTTTGCACACAGCTGAACA |
| | GAGCCCTGACTGGGATCGCTGTGGAGCAGGACAAGAACAC |
| | ACAGGAGGTGTTTGCACAGGTGAAGCAGATCTATAAGACC |
| | CCTCCTATTAAGGATTTCGGCGGATTCAATTTCTCACAGAT |
| | TCTGCCAGACCCCAGTAAGCCTTCCAAGAGGAGCTTCATCG |
| | AGGATCTCCTGTTTAACAAGGTGACCCTGGCAGACGCCGGC |
| | TTTATTAAGCAATATGGGGATTGCCTGGGCGACATTGCTGC |
| | CAGAGACCTGATTTGCGCCCAGAAATTCAATGGCCTCACAG |
| | TGCTGCCACCTCTGCTGACCGACGAGATGATCGCTCAATAC |
| | ACTAGCGCACTGCTGGCCGGAACCATCACATCAGGCTGGA |
| | CCTTCGGGGCCGGAGCAGCACTGCAGATTCCATTCGCCATG |
| | CAGATGGCCTATAGATTCAACGGCATTGGCGTCACACAGA |
| | ACGTGCTGTACGAAAACCAGAAGCTCATCGCTAACCAGTTT |
| | AATTCCGCAATTGGAAAGATCCAAGATTCACTCAGCTCAAC |
| | CGCCTCTGCACTCGGAAAGCTGCAGGACGTGGTCAACCAG |
| | AATGCTCAGGCCCTGAACACACTCGTCAAGCAGCTGTCCTC |
| | TAACTTTGGCGCTATCAGCTCCGTTCTGAACGACATTCTGA |
| | GCCGCCTGGATAAGGTGGAGGCTGAAGTCCAGATTGACCG |
| | CCTGATTACCGGCCGGCTGCAGTCTCTGCAAACATACGTGA |
| | CCCAGCAGCTGATCAGAGCAGCCGAGATCCGGGCATCCGC |
| | AAATCTGGCAGCAACTAAGATGAGCGAATGCGTGCTGGGC |
| | CAGTCCAAGCGGGTGGACTTTTGTGGCAAGGGCTACCACCT |
| | GATGAGCTTCCCCCAGAGCGCCCCACATGGCGTTGTTTTTC |
| | TGCACGTGACCTATGTCCCTGCTCAGGAAAAGAACTTTACA |
| | ACTGCTCCTGCTATCTGCCATGACGGCAAGGCCCACTTCCC |
| | ACGGGAGGGAGTGTTTGTGTCCAATGGCACACACTGGTTCG |
| | TGACCCAGAGGAACTTCTATGAACCCCAGATCATCACCACT |
| | GACAATACCTTCGTGTCTGGAAATTGCGACGTCGTGATCGG |
| | CATCGTTAACAACACCGTGTACGACCCTCTCCAGCCAGAGC |
| | TGGACTCCTTTAAGGAGGAACTGGATAAGTATTTTAAGAAC |
| | CACACAAGCCCAGATGTGGATCTCGGGGACATCTCCGGAA |
| | TTAACGCCTCCGTGGTGAATATCCAGAAGGAGATTGACCGC |
| | CTAAATGAAGTTGCCAAGAACCTCAATGAGTCTCTGATTGA |
| | TCTGCAGGAACTGGGCAAGTATGAGCAGTATATCAAATGG |
| | CCCTGGTACATTTGGCTGGGGTTTATCGCCGGACTGATTGC |
| | CATCGTCATGGTGACCATCATGCTGTGTTGCATGACCTCCT |
| | GTTGTTCCTGTCTGAAGGGCTGCTGTAGTTGCGGCTCTTGCT |
| | GTAAATTCGACGAAGATGATAGCGAGCCCGTGCTGAAGGG |
| | CGTGAAGCTGCATTATACCTGA |
| SARS-CoV-2 S protein | (SEQ ID NO: 165) |
| containing the L18F, | MFVFLVLLPLVSSQCVNFTTRTQLPPAYTNSFTRGVYYPDKVF |
| D80A, D215G, L242-, | RSSVLHSTQDLFLPFFSNVTWFHAIHVSGTNGTKRFANPVLPF |
| A243-, L244-, K417N, | NDGVYFASTEKSNIIRGWIFGTTLDSKTQSLLIVNNATNVVIKV |
| E484K, N501Y, D614G | CEFQFCNDPFLGVYYHKNNKSWMESEFRVYSSANNCTFEYVS |
| and A701V mutations | QPFLMDLEGKQGNFKNLREFVFKNIDGYFKIYSKHTPINLVRG |
| | LPQGFSALEPLVDLPIGINITRFQTLHRSYLTPGDSSSGWTAGA |
| | AAYYVGYLQPRTFLLKYNENGTITDAVDCALDPLSETKCTLK |
| | SFTVEKGIYQTSNFRVQPTESIVRFPNITNLCPFGEVENATRFAS |
| | VYAWNRKRISNCVADYSVLYNSASFSTFKCYGVSPTKLNDLC |
| | FTNVYADSFVIRGDEVRQIAPGQTGNIADYNYKLPDDFTGCVI |
| | AWNSNNLDSKVGGNYNYLYRLFRKSNLKPFERDISTEIYQAG |
| | STPCNGVKGFNCYFPLQSYGFQPTYGVGYQPYRVVVLSFELL |
| | HAPATVCGPKKSTNLVKNKCVNFNFNGLTGTGVLTESNKKFL |
| | PFQQFGRDIADTTDAVRDPQTLEILDITPCSFGGVSVITPGTNTS |
| | NQVAVLYQGVNCTEVPVAIHADQLTPTWRVYSTGSNVFQTR |
| | AGCLIGAEHVNNSYECDIPIGAGICASYQTQTNSPRRARSVAS |
| | QSIIAYTMSLGVENSVAYSNNSIAIPTNFTISVTTEILPVSMTKT |
| | SVDCTMYICGDSTECSNLLLQYGSFCTQLNRALTGIAVEQDK |
| | NTQEVFAQVKQIYKTPPIKDFGGFNFSQILPDPSKPSKRSFIEDL |
| | LFNKVTLADAGFIKQYGDCLGDIAARDLICAQKFNGLTVLPPL |
| | LTDEMIAQYTSALLAGTITSGWTFGAGAALQIPFAMQMAYRF |
| | NGIGVTQNVLYENQKLIANQFNSAIGKIQDSLSSTASALGKLQ |
| | DVVNQNAQALNTLVKQLSSNFGAISSVLNDILSRLDKVEAEV |
| | QIDRLITGRLQSLQTYVTQQLIRAAEIRASANLAATKMSECVL |
| | GQSKRVDFCGKGYHLMSFPQSAPHGVVFLHVTYVPAQEKNF |
| | TTAPAICHDGKAHFPREGVFVSNGTHWFVTQRNFYEPQUITTD |
| | NTFVSGNCDVVIGIVNNTVYDPLQPELDSFKEELDKYFKNHTS |
| | PDVDLGDISGINASVVNIQKEIDRLNEVAKNLNESLIDLQELGK |
| | YEQYIKWPWYIWLGFIAGLIAIVMVTIMLCCMTSCCSCLKGCC |
| | SCGSCCKFDEDDSEPVLKGVKLHYT |
| Optimized nucleotide | (SEQ ID NO: 166) |
| sequence encoding a | ATGTTCGTCTTCCTCGTGCTGCTCCCACTCGTTTCTTCCCAG |
| SARS-CoV-2 S protein | TGTGTCAACTTCACAACTAGGACTCAGCTGCCACCAGCCTA |
| containing mutated to | CACCAACTCCTTCACCAGAGGCGTGTATTACCCAGACAAGG |
| remove a furin cleavage | TGTTTAGAAGCAGCGTGCTGCACTCTACCCAGGACCTCTTT |
| site and to replace | CTGCCCTTTTTCAGCAACGTGACATGGTTTCACGCAATTCA |
| residues 986 and 987 | CGTGTCCGGCACTAATGGCACAAAGCGGTTCGCCAATCCA |
| with proline and which | GTCCTGCCTTTCAACGATGGCGTCTACTTTGCATCTACTGA |
| contains the L18F, | GAAATCCAATATCATTAGGGGATGGATCTTCGGCACAACCC |
| D80A, D215G, L242-, | TGGATTCTAAGACCCAGAGCCTGCTGATCGTCAACAACGCC |
| A243-, L244-, K417N, | ACAAACGTGGTCATTAAGGTTTGCGAGTTTCAGTTCTGTAA |
| E484K, N501Y, D614G | CGATCCTTTTCTGGGCGTGTATTATCATAAGAACAATAAGA |
| and A701V mutations | GCTGGATGGAGTCCGAGTTTAGAGTGTATAGCTCTGCAAAT |
| | AATTGTACCTTTGAGTACGTGAGCCAGCCCTTTCTGATGGA |
| | CCTGGAGGGAAAACAAGGAAACTTCAAAAACCTGCGGGAA |
| | TTCGTTTTCAAAAACATCGACGGCTATTTCAAGATCTATAG |
| | CAAGCATACCCCAATCAACCTCGTGAGGGGCCTCCCCCAG |
| | GGCTTTAGCGCACTGGAGCCACTGGTTGACCTGCCTATCGG |
| | CATTAATATCACAAGATTTCAGACCCTGCATAGAAGCTATC |
| | TGACCCCTGGAGACTCCTCTAGTGGGTGGACTGCCGGCGCC |
| | GCTGCCTACTATGTGGGCTATCTGCAGCCACGGACATTCCT |
| | GCTGAAATACAATGAGAACGGGACAATCACAGATGCTGTT |
| | GATTGCGCACTCGACCCCCTGTCCGAGACAAAGTGCACTCT |
| | CAAGAGCTTTACCGTCGAGAAGGGCATCTATCAGACCTCA |
| | AACTTCAGGGTGCAGCCCACAGAATCTATCGTGCGCTTCCC |
| | TAATATCACTAACCTGTGTCCTTTCGGTGAAGTGTTCAACG |
| | CCACCAGGTTTGCTAGCGTGTATGCCTGGAACAGGAAGAG |
| | GATCTCTAACTGCGTCGCCGACTATTCCGTGCTGTATAACA |
| | GCGCCTCCTTCTCCACATTCAAATGCTATGGAGTGAGCCCG |
| | ACAAAACTGAACGATCTCTGCTTTACAAATGTCTACGCCGA |
| | CTCTTTTGTGATCAGAGGGGACGAGGTCCGGCAGATCGCAC |
| | CAGGACAGACAGGCAACATTGCTGACTACAACTATAAGCT |
| | GCCTGACGACTTCACAGGATGTGTGATCGCATGGAACTCAA |
| | ACAATCTGGACTCCAAAGTCGGGGGCAACTATAATTACCTG |
| | TATCGCCTGTTCCGGAAGTCCAACCTGAAGCCCTTCGAGAG |
| | GGACATCAGTACAGAGATCTATCAGGCTGGCTCCACCCCTT |
| | GCAATGGCGTCAAGGGCTTTAATTGTTATTTTCCCCTGCAG |
| | TCTTACGGGTTTCAGCCTACTTACGGAGTTGGGTACCAGCC |
| | ATACAGAGTGGTCGTGCTCAGCTTCGAGCTCCTGCATGCTC |
| | CAGCTACAGTTTGCGGGCCAAAGAAGTCCACTAACCTGGT |
| | GAAGAATAAGTGCGTCAACTTCAACTTTAACGGGCTCACCG |
| | GCACCGGCGTGCTGACTGAGAGCAACAAGAAGTTTCTGCC |
| | ATTTCAACAGTTTGGACGGGACATTGCCGACACCACCGATG |
| | CCGTTCGGGATCCACAGACCCTGGAAATTCTGGACATTACA |
| | CCGTGCAGCTTCGGGGGCGTGAGCGTGATCACACCCGGAA |
| | CCAATACAAGCAACCAGGTTGCCGTCCTGTATCAGGGCGT |
| | CAATTGCACAGAAGTGCCAGTTGCTATCCACGCAGACCAG |
| | CTGACTCCCACATGGCGGGTGTATAGCACCGGATCCAACGT |
| | GTTTCAGACCCGCGCCGGATGTCTCATTGGGGCCGAGCACG |
| | TGAATAACAGCTACGAGTGCGACATCCCCATTGGCGCCGG |
| | CATTTGTGCGTCTTACCAGACTCAGACCAACTCTCCTGGCT |
| | CCGCCTCTTCCGTTGCTAGTCAGTCTATTATTGCCTATACCA |
| | TGAGCCTCGGAGTGGAGAATAGCGTGGCCTACTCCAATAA |
| | TTCCATCGCAATCCCTACTAACTTCACTATTTCTGTGACCAC |
| | CGAGATCCTGCCTGTGTCTATGACTAAGACTAGCGTTGATT |
| | GTACCATGTATATTTGTGGCGACTCTACCGAATGTTCTAAC |
| | CTGCTGCTTCAGTACGGCTCATTTTGCACACAGCTGAACAG |
| | AGCCCTGACTGGGATCGCTGTGGAGCAGGACAAGAACACA |
| | CAGGAGGTGTTTGCACAGGTGAAGCAGATCTATAAGACCC |
| | CTCCTATTAAGGATTTCGGCGGATTCAATTTCTCACAGATT |
| | CTGCCAGACCCCAGTAAGCCTTCCAAGAGGAGCTTCATCGA |
| | GGATCTCCTGTTTAACAAGGTGACCCTGGCAGACGCCGGCT |
| | TTATTAAGCAATATGGGGATTGCCTGGGCGACATTGCTGCC |
| | AGAGACCTGATTTGCGCCCAGAAATTCAATGGCCTCACAGT |
| | GCTGCCACCTCTGCTGACCGACGAGATGATCGCTCAATACA |
| | CTAGCGCACTGCTGGCCGGAACCATCACATCAGGCTGGAC |
| | CTTCGGGGCCGGAGCAGCACTGCAGATTCCATTCGCCATGC |
| | AGATGGCCTATAGATTCAACGGCATTGGCGTCACACAGAA |
| | CGTGCTGTACGAAAACCAGAAGCTCATCGCTAACCAGTTTA |
| | ATTCCGCAATTGGAAAGATCCAAGATTCACTCAGCTCAACC |
| | GCCTCTGCACTCGGAAAGCTGCAGGACGTGGTCAACCAGA |
| | ATGCTCAGGCCCTGAACACACTCGTCAAGCAGCTGTCCTCT |
| | AACTTTGGCGCTATCAGCTCCGTTCTGAACGACATTCTGAG |
| | CCGCCTGGATCCCCCAGAGGCTGAAGTCCAGATTGACCGCC |
| | TGATTACCGGCCGGCTGCAGTCTCTGCAAACATACGTGACC |
| | CAGCAGCTGATCAGAGCAGCCGAGATCCGGGCATCCGCAA |
| | ATCTGGCAGCAACTAAGATGAGCGAATGCGTGCTGGGCCA |
| | GTCCAAGCGGGTGGACTTTTGTGGCAAGGGCTACCACCTGA |
| | TGAGCTTCCCCCAGAGCGCCCCACATGGCGTTGTTTTTCTG |
| | CACGTGACCTATGTCCCTGCTCAGGAAAAGAACTTTACAAC |
| | TGCTCCTGCTATCTGCCATGACGGCAAGGCCCACTTCCCAC |
| | GGGAGGGAGTGTTTGTGTCCAATGGCACACACTGGTTCGTG |
| | ACCCAGAGGAACTTCTATGAACCCCAGATCATCACCACTGA |
| | CAATACCTTCGTGTCTGGAAATTGCGACGTCGTGATCGGCA |
| | TCGTTAACAACACCGTGTACGACCCTCTCCAGCCAGAGCTG |
| | GACTCCTTTAAGGAGGAACTGGATAAGTATTTTAAGAACCA |
| | CACAAGCCCAGATGTGGATCTCGGGGACATCTCCGGAATT |
| | AACGCCTCCGTGGTGAATATCCAGAAGGAGATTGACCGCC |
| | TAAATGAAGTTGCCAAGAACCTCAATGAGTCTCTGATTGAT |
| | CTGCAGGAACTGGGCAAGTATGAGCAGTATATCAAATGGC |
| | CCTGGTACATTTGGCTGGGGTTTATCGCCGGACTGATTGCC |
| | ATCGTCATGGTGACCATCATGCTGTGTTGCATGACCTCCTG |
| | TTGTTCCTGTCTGAAGGGCTGCTGTAGTTGCGGCTCTTGCTG |
| | TAAATTCGACGAAGATGATAGCGAGCCCGTGCTGAAGGGC |
| | GTGAAGCTGCATTATACCTGA |
| SARS-CoV-2 S protein | (SEQ ID NO: 167) |
| containing mutated to | MFVFLVLLPLVSSQCVNFTTRTQLPPAYTNSFTRGVYYPDKVF |
| remove a furin cleavage | RSSVLHSTQDLFLPFFSNVTWFHAIHVSGTNGTKRFANPVLPF |
| site and to replace | NDGVYFASTEKSNIIRGWIFGTTLDSKTQSLLIVNNATNVVIKV |
| residues 986 and 987 | CEFQFCNDPFLGVYYHKNNKSWMESEFRVYSSANNCTFEYVS |
| with proline and which | QPFLMDLEGKQGNFKNLREFVFKNIDGYFKIYSKHTPINLVRG |
| contains the L18F, | LPQGFSALEPLVDLPIGINITRFQTLHRSYLTPGDSSSGWTAGA |
| D80A, D215G, L242-, | AAYYVGYLQPRTFLLKYNENGTITDAVDCALDPLSETKCTLK |
| A243-, L244-, K417N, | SFTVEKGIYQTSNFRVQPTESIVRFPNITNLCPFGEVFNATRFAS |
| E484K, N501Y, D614G | VYAWNRKRISNCVADYSVLYNSASFSTFKCYGVSPTKLNDLC |
| and A701V mutations | FTNVYADSFVIRGDEVRQIAPGQTGNIADYNYKLPDDFTGCVI |
| | AWNSNNLDSKVGGNYNYLYRLFRKSNLKPFERDISTEIYQAG |
| | STPCNGVKGFNCYFPLQSYGFQPTYGVGYQPYRVVVLSFELL |
| | HAPATVCGPKKSTNLVKNKCVNFNFNGLTGTGVLTESNKKFL |
| | PFQQFGRDIADTTDAVRDPQTLEILDITPCSFGGVSVITPGTNTS |
| | NQVAVLYQGVNCTEVPVAIHADQLTPTWRVYSTGSNVFQTR |
| | AGCLIGAEHVNNSYECDIPIGAGICASYQTQTNSPGSASSVASQ |
| | SIIAYTMSLGVENSVAYSNNSIAIPTNFTISVTTEILPVSMTKTS |
| | VDCTMYICGDSTECSNLLLQYGSFCTQLNRALTGIAVEQDKN |
| | TQEVFAQVKQIYKTPPIKDFGGFNFSQILPDPSKPSKRSFIEDLL |
| | FNKVTLADAGFIKQYGDCLGDIAARDLICAQKFNGLTVLPPLL |
| | TDEMIAQYTSALLAGTITSGWTFGAGAALQIPFAMQMAYRFN |
| | GIGVTQNVLYENQKLIANQFNSAIGKIQDSLSSTASALGKLQD |
| | VVNQNAQALNTLVKQLSSNFGAISSVLNDILSRLDPPEAEVQI |
| | DRLITGRLQSLQTYVTQQLIRAAEIRASANLAATKMSECVLGQ |
| | SKRVDFCGKGYHLMSFPQSAPHGVVFLHVTYVPAQEKNFTTA |
| | PAICHDGKAHFPREGVFVSNGTHWFVTQRNFYEPQIITTDNTF |
| | VSGNCDVVIGIVNNTVYDPLQPELDSFKEELDKYFKNHTSPDV |
| | DLGDISGINASVVNIQKEIDRLNEVAKNLNESLIDLQELGKYEQ |
| | YIKWPWYIWLGFIAGLIAIVMVTIMLCCMTSCCSCLKGCCSCG |
| | SCCKFDEDDSEPVLKGVKLHYT |
| Optimized nucleotide | (SEQ ID NO: 168) |
| sequence encoding a | ATGTTCGTCTTCCTCGTGCTGCTCCCACTCGTTTCTTCCCAG |
| SARS-CoV-2 S protein | TGTGTCAACTTTACAAACAGGACTCAGCTGCCATCCGCCT |
| containing the L18F, | ACACCAACTCCTTCACCAGAGGCGTGTATTACCCAGACAAG |
| T20N, P26S, D138Y, | GTGTTTAGAAGCAGCGTGCTGCACTCTACCCAGGACCTCTT |
| R190S, K417T, E484K, | TCTGCCCTTTTTCAGCAACGTGACATGGTTTCACGCAATTC |
| N501Y, D614G, H655Y, | ACGTGTCCGGCACTAATGGCACAAAGCGGTTCGACAATCC |
| T10271 and V1176F | AGTCCTGCCTTTCAACGATGGCGTCTACTTTGCATCTACTG |
| mutations | AGAAATCCAATATCATTAGGGGATGGATCTTCGGCACAAC |
| | CCTGGATTCTAAGACCCAGAGCCTGCTGATCGTCAACAACG |
| | CCACAAACGTGGTCATTAAGGTTTGCGAGTTTCAGTTCTGT |
| | AACTACCCTTTTCTGGGCGTGTATTATCATAAGAACAATAA |
| | GAGCTGGATGGAGTCCGAGTTTAGAGTGTATAGCTCTGCAA |
| | ATAATTGTACCTTTGAGTACGTGAGCCAGCCCTTTCTGATG |
| | GACCTGGAGGGAAAACAAGGAAACTTCAAAAACCTGAGC |
| | GAATTCGTTTTCAAAAACATCGACGGCTATTTCAAGATCTA |
| | TAGCAAGCATACCCCAATCAACCTCGTGAGGGACCTCCCCC |
| | AGGGCTTTAGCGCACTGGAGCCACTGGTTGACCTGCCTATC |
| | GGCATTAATATCACAAGATTTCAGACCCTGCTGGCACTGCA |
| | TAGAAGCTATCTGACCCCTGGAGACTCCTCTAGTGGGTGGA |
| | CTGCCGGCGCCGCTGCCTACTATGTGGGCTATCTGCAGCCA |
| | CGGACATTCCTGCTGAAATACAATGAGAACGGGACAATCA |
| | CAGATGCTGTTGATTGCGCACTCGACCCCCTGTCCGAGACA |
| | AAGTGCACTCTCAAGAGCTTTACCGTCGAGAAGGGCATCTA |
| | TCAGACCTCAAACTTCAGGGTGCAGCCCACAGAATCTATCG |
| | TGCGCTTCCCTAATATCACTAACCTGTGTCCTTTCGGTGAA |
| | GTGTTCAACGCCACCAGGTTTGCTAGCGTGTATGCCTGGAA |
| | CAGGAAGAGGATCTCTAACTGCGTCGCCGACTATTCCGTGC |
| | TGTATAACAGCGCCTCCTTCTCCACATTCAAATGCTATGGA |
| | GTGAGCCCGACAAAACTGAACGATCTCTGCTTTACAAATGT |
| | CTACGCCGACTCTTTTGTGATCAGAGGGGACGAGGTCCGGC |
| | AGATCGCACCAGGACAGACAGGCACCATTGCTGACTACAA |
| | CTATAAGCTGCCTGACGACTTCACAGGATGTGTGATCGCAT |
| | GGAACTCAAACAATCTGGACTCCAAAGTCGGGGGCAACTA |
| | TAATTACCTGTATCGCCTGTTCCGGAAGTCCAACCTGAAGC |
| | CCTTCGAGAGGGACATCAGTACAGAGATCTATCAGGCTGG |
| | CTCCACCCCTTGCAATGGCGTCAAGGGCTTTAATTGTTATT |
| | TTCCCCTGCAGTCTTACGGGTTTCAGCCTACTTACGGAGTT |
| | GGGTACCAGCCATACAGAGTGGTCGTGCTCAGCTTCGAGCT |
| | CCTGCATGCTCCAGCTACAGTTTGCGGGCCAAAGAAGTCCA |
| | CTAACCTGGTGAAGAATAAGTGCGTCAACTTCAACTTTAAC |
| | GGGCTCACCGGCACCGGCGTGCTGACTGAGAGCAACAAGA |
| | AGTTTCTGCCATTTCAACAGTTTGGACGGGACATTGCCGAC |
| | ACCACCGATGCCGTTCGGGATCCACAGACCCTGGAAATTCT |
| | GGACATTACACCGTGCAGCTTCGGGGGCGTGAGCGTGATC |
| | ACACCCGGAACCAATACAAGCAACCAGGTTGCCGTCCTGT |
| | ATCAGGGCGTCAATTGCACAGAAGTGCCAGTTGCTATCCA |
| | CGCAGACCAGCTGACTCCCACATGGCGGGTGTATAGCACC |
| | GGATCCAACGTGTTTCAGACCCGCGCCGGATGTCTCATTGG |
| | GGCCGAGTACGTGAATAACAGCTACGAGTGCGACATCCCC |
| | ATTGGCGCCGGCATTTGTGCGTCTTACCAGACTCAGACCAA |
| | CTCTCCTAGAAGGGCCAGGTCCGTTGCTAGTCAGTCTATTA |
| | TTGCCTATACCATGAGCCTCGGAGCTGAGAATAGCGTGGCC |
| | TACTCCAATAATTCCATCGCAATCCCTACTAACTTCACTATT |
| | TCTGTGACCACCGAGATCCTGCCTGTGTCTATGACTAAGAC |
| | TAGCGTTGATTGTACCATGTATATTTGTGGCGACTCTACCG |
| | AATGTTCTAACCTGCTGCTTCAGTACGGCTCATTTTGCACA |
| | CAGCTGAACAGAGCCCTGACTGGGATCGCTGTGGAGCAGG |
| | ACAAGAACACACAGGAGGTGTTTGCACAGGTGAAGCAGAT |
| | CTATAAGACCCCTCCTATTAAGGATTTCGGCGGATTCAATT |
| | TCTCACAGATTCTGCCAGACCCCAGTAAGCCTTCCAAGAGG |
| | AGCTTCATCGAGGATCTCCTGTTTAACAAGGTGACCCTGGC |
| | AGACGCCGGCTTTATTAAGCAATATGGGGATTGCCTGGGCG |
| | ACATTGCTGCCAGAGACCTGATTTGCGCCCAGAAATTCAAT |
| | GGCCTCACAGTGCTGCCACCTCTGCTGACCGACGAGATGAT |
| | CGCTCAATACACTAGCGCACTGCTGGCCGGAACCATCACAT |
| | CAGGCTGGACCTTCGGGGCCGGAGCAGCACTGCAGATTCC |
| | ATTCGCCATGCAGATGGCCTATAGATTCAACGGCATTGGCG |
| | TCACACAGAACGTGCTGTACGAAAACCAGAAGCTCATCGC |
| | TAACCAGTTTAATTCCGCAATTGGAAAGATCCAAGATTCAC |
| | TCAGCTCAACCGCCTCTGCACTCGGAAAGCTGCAGGACGTG |
| | GTCAACCAGAATGCTCAGGCCCTGAACACACTCGTCAAGC |
| | AGCTGTCCTCTAACTTTGGCGCTATCAGCTCCGTTCTGAAC |
| | GACATTCTGAGCCGCCTGGATAAGGTGGAGGCTGAAGTCC |
| | AGATTGACCGCCTGATTACCGGCCGGCTGCAGTCTCTGCAA |
| | ACATACGTGACCCAGCAGCTGATCAGAGCAGCCGAGATCC |
| | GGGCATCCGCAAATCTGGCAGCAATCAAGATGAGCGAATG |
| | CGTGCTGGGCCAGTCCAAGCGGGTGGACTTTTGTGGCAAG |
| | GGCTACCACCTGATGAGCTTCCCCCAGAGCGCCCCACATGG |
| | CGTTGTTTTTCTGCACGTGACCTATGTCCCTGCTCAGGAAA |
| | AGAACTTTACAACTGCTCCTGCTATCTGCCATGACGGCAAG |
| | GCCCACTTCCCACGGGAGGGAGTGTTTGTGTCCAATGGCAC |
| | ACACTGGTTCGTGACCCAGAGGAACTTCTATGAACCCCAGA |
| | TCATCACCACTGACAATACCTTCGTGTCTGGAAATTGCGAC |
| | GTCGTGATCGGCATCGTTAACAACACCGTGTACGACCCTCT |
| | CCAGCCAGAGCTGGACTCCTTTAAGGAGGAACTGGATAAG |
| | TATTTTAAGAACCACACAAGCCCAGATGTGGATCTCGGGG |
| | ACATCTCCGGAATTAACGCCTCCTTCGTGAATATCCAGAAG |
| | GAGATTGACCGCCTAAATGAAGTTGCCAAGAACCTCAATG |
| | AGTCTCTGATTGATCTGCAGGAACTGGGCAAGTATGAGCA |
| | GTATATCAAATGGCCCTGGTACATTTGGCTGGGGTTTATCG |
| | CCGGACTGATTGCCATCGTCATGGTGACCATCATGCTGTGT |
| | TGCATGACCTCCTGTTGTTCCTGTCTGAAGGGCTGCTGTAG |
| | TTGCGGCTCTTGCTGTAAATTCGACGAAGATGATAGCGAGC |
| | CCGTGCTGAAGGGCGTGAAGCTGCATTATACCTGA |
| SARS-CoV-2 S protein | (SEQ ID NO: 169) |
| containing the L18F, | MFVFLVLLPLVSSQCVNFTNRTQLPSAYTNSFTRGVYYPDKV |
| T20N, P26S, D138Y, | FRSSVLHSTQDLFLPFFSNVTWFHAIHVSGTNGTKRFDNPVLP |
| R190S, K417T, E484K, | FNDGVYFASTEKSNIIRGWIFGTTLDSKTQSLLIVNNATNVVIK |
| N501Y, D614G, H655Y, | VCEFQFCNYPFLGVYYHKNNKSWMESEFRVYSSANNCTFEY |
| T10271 and V1176F | VSQPFLMDLEGKQGNFKNLSEFVFKNIDGYFKIYSKHTPINLV |
| mutations | RDLPQGFSALEPLVDLPIGINITRFQTLLALHRSYLTPGDSSSG |
| | WTAGAAAYYVGYLQPRTFLLKYNENGTITDAVDCALDPLSET |
| | KCTLKSFTVEKGIYQTSNFRVQPTESIVRFPNITNLCPFGEVFN |
| | ATRFASVYAWNRKRISNCVADYSVLYNSASFSTFKCYGVSPT |
| | KLNDLCFTNVYADSFVIRGDEVRQIAPGQTGTIADYNYKLPD |
| | DFTGCVIAWNSNNLDSKVGGNYNYLYRLFRKSNLKPFERDIS |
| | TEIYQAGSTPCNGVKGFNCYFPLQSYGFQPTYGVGYQPYRVV |
| | VLSFELLHAPATVCGPKKSTNLVKNKCVNFNFNGLTGTGVLT |
| | ESNKKFLPFQQFGRDIADTTDAVRDPQTLEILDITPCSFGGVSV |
| | ITPGTNTSNQVAVLYQGVNCTEVPVAIHADQLTPTWRVYSTG |
| | SNVFQTRAGCLIGAEYVNNSYECDIPIGAGICASYQTQTNSPRR |
| | ARSVASQSIIAYTMSLGAENSVAYSNNSIAIPTNFTISVTTEILP |
| | VSMTKTSVDCTMYICGDSTECSNLLLQYGSFCTQLNRALTGIA |
| | VEQDKNTQEVFAQVKQIYKTPPIKDFGGFNFSQILPDPSKPSKR |
| | SFIEDLLFNKVTLADAGFIKQYGDCLGDIAARDLICAQKFNGL |
| | TVLPPLLTDEMIAQYTSALLAGTITSGWTFGAGAALQIPFAMQ |
| | MAYRFNGIGVTQNVLYENQKLIANQFNSAIGKIQDSLSSTASA |
| | LGKLQDVVNQNAQALNTLVKQLSSNFGAISSVLNDILSRLDK |
| | VEAEVQIDRLITGRLQSLQTYVTQQLIRAAEIRASANLAAIKM |
| | SECVLGQSKRVDFCGKGYHLMSFPQSAPHGVVFLHVTYVPA |
| | QEKNFTTAPAICHDGKAHFPREGVFVSNGTHWFVTQRNFYEP |
| | QIITTDNTFVSGNCDVVIGIVNNTVYDPLQPELDSFKEELDKYF |
| | KNHTSPDVDLGDISGINASFVNIQKEIDRLNEVAKNLNESLIDL |
| | QELGKYEQYIKWPWYIWLGFIAGLIAIVMVTIMLCCMTSCCSC |
| | LKGCCSCGSCCKFDEDDSEPVLKGVKLHYT |
| Optimized nucleotide | (SEQ ID NO: 170) |
| sequence encoding a | ATGTTCGTCTTCCTCGTGCTGCTCCCACTCGTTTCTTCCCAG |
| SARS-CoV-2 S protein | TGTGTCAACTTTACAAACAGGACTCAGCTGCCATCCGCCT |
| mutated to remove a | ACACCAACTCCTTCACCAGAGGCGTGTATTACCCAGACAAG |
| furin cleavage site and | GTGTTTAGAAGCAGCGTGCTGCACTCTACCCAGGACCTCTT |
| to replace residues 986 | TCTGCCCTTTTTCAGCAACGTGACATGGTTTCACGCAATTC |
| and 987 with proline | ACGTGTCCGGCACTAATGGCACAAAGCGGTTCGACAATCC |
| and which contains the | AGTCCTGCCTTTCAACGATGGCGTCTACTTTGCATCTACTG |
| L18F, T20N, P26S, | AGAAATCCAATATCATTAGGGGATGGATCTTCGGCACAAC |
| D138Y, R190S, K417T, | CCTGGATTCTAAGACCCAGAGCCTGCTGATCGTCAACAACG |
| E484K, N501Y, D614G, | CCACAAACGTGGTCATTAAGGTTTGCGAGTTTCAGTTCTGT |
| H655Y, T1027I and | AACTACCCTTTTCTGGGCGTGTATTATCATAAGAACAATAA |
| V1176F mutations | GAGCTGGATGGAGTCCGAGTTTAGAGTGTATAGCTCTGCAA |
| | ATAATTGTACCTTTGAGTACGTGAGCCAGCCCTTTCTGATG |
| | GACCTGGAGGGAAAACAAGGAAACTTCAAAAACCTGAGC |
| | GAATTCGTTTTCAAAAACATCGACGGCTATTTCAAGATCTA |
| | TAGCAAGCATACCCCAATCAACCTCGTGAGGGACCTCCCCC |
| | AGGGCTTTAGCGCACTGGAGCCACTGGTTGACCTGCCTATC |
| | GGCATTAATATCACAAGATTTCAGACCCTGCTGGCACTGCA |
| | TAGAAGCTATCTGACCCCTGGAGACTCCTCTAGTGGGTGGA |
| | CTGCCGGCGCCGCTGCCTACTATGTGGGCTATCTGCAGCCA |
| | CGGACATTCCTGCTGAAATACAATGAGAACGGGACAATCA |
| | CAGATGCTGTTGATTGCGCACTCGACCCCCTGTCCGAGACA |
| | AAGTGCACTCTCAAGAGCTTTACCGTCGAGAAGGGCATCTA |
| | TCAGACCTCAAACTTCAGGGTGCAGCCCACAGAATCTATCG |
| | TGCGCTTCCCTAATATCACTAACCTGTGTCCTTTCGGTGAA |
| | GTGTTCAACGCCACCAGGTTTGCTAGCGTGTATGCCTGGAA |
| | CAGGAAGAGGATCTCTAACTGCGTCGCCGACTATTCCGTGC |
| | TGTATAACAGCGCCTCCTTCTCCACATTCAAATGCTATGGA |
| | GTGAGCCCGACAAAACTGAACGATCTCTGCTTTACAAATGT |
| | CTACGCCGACTCTTTTGTGATCAGAGGGGACGAGGTCCGGC |
| | AGATCGCACCAGGACAGACAGGCACCATTGCTGACTACAA |
| | CTATAAGCTGCCTGACGACTTCACAGGATGTGTGATCGCAT |
| | GGAACTCAAACAATCTGGACTCCAAAGTCGGGGGCAACTA |
| | TAATTACCTGTATCGCCTGTTCCGGAAGTCCAACCTGAAGC |
| | CCTTCGAGAGGGACATCAGTACAGAGATCTATCAGGCTGG |
| | CTCCACCCCTTGCAATGGCGTCAAGGGCTTTAATTGTTATT |
| | TTCCCCTGCAGTCTTACGGGTTTCAGCCTACTTACGGAGTT |
| | GGGTACCAGCCATACAGAGTGGTCGTGCTCAGCTTCGAGCT |
| | CCTGCATGCTCCAGCTACAGTTTGCGGGCCAAAGAAGTCCA |
| | CTAACCTGGTGAAGAATAAGTGCGTCAACTTCAACTTTAAC |
| | GGGCTCACCGGCACCGGCGTGCTGACTGAGAGCAACAAGA |
| | AGTTTCTGCCATTTCAACAGTTTGGACGGGACATTGCCGAC |
| | ACCACCGATGCCGTTCGGGATCCACAGACCCTGGAAATTCT |
| | GGACATTACACCGTGCAGCTTCGGGGGCGTGAGCGTGATC |
| | ACACCCGGAACCAATACAAGCAACCAGGTTGCCGTCCTGT |
| | ATCAGGGCGTCAATTGCACAGAAGTGCCAGTTGCTATCCA |
| | CGCAGACCAGCTGACTCCCACATGGCGGGTGTATAGCACC |
| | GGATCCAACGTGTTTCAGACCCGCGCCGGATGTCTCATTGG |
| | GGCCGAGTACGTGAATAACAGCTACGAGTGCGACATCCCC |
| | ATTGGCGCCGGCATTTGTGCGTCTTACCAGACTCAGACCAA |
| | CTCTCCTGGCTCCGCCTCTTCCGTTGCTAGTCAGTCTATTAT |
| | TGCCTATACCATGAGCCTCGGAGCTGAGAATAGCGTGGCCT |
| | ACTCCAATAATTCCATCGCAATCCCTACTAACTTCACTATTT |
| | CTGTGACCACCGAGATCCTGCCTGTGTCTATGACTAAGACT |
| | AGCGTTGATTGTACCATGTATATTTGTGGCGACTCTACCGA |
| | ATGTTCTAACCTGCTGCTTCAGTACGGCTCATTTTGCACAC |
| | AGCTGAACAGAGCCCTGACTGGGATCGCTGTGGAGCAGGA |
| | CAAGAACACACAGGAGGTGTTTGCACAGGTGAAGCAGATC |
| | TATAAGACCCCTCCTATTAAGGATTTCGGCGGATTCAATTT |
| | CTCACAGATTCTGCCAGACCCCAGTAAGCCTTCCAAGAGGA |
| | GCTTCATCGAGGATCTCCTGTTTAACAAGGTGACCCTGGCA |
| | GACGCCGGCTTTATTAAGCAATATGGGGATTGCCTGGGCGA |
| | CATTGCTGCCAGAGACCTGATTTGCGCCCAGAAATTCAATG |
| | GCCTCACAGTGCTGCCACCTCTGCTGACCGACGAGATGATC |
| | GCTCAATACACTAGCGCACTGCTGGCCGGAACCATCACATC |
| | AGGCTGGACCTTCGGGGCCGGAGCAGCACTGCAGATTCCA |
| | TTCGCCATGCAGATGGCCTATAGATTCAACGGCATTGGCGT |
| | CACACAGAACGTGCTGTACGAAAACCAGAAGCTCATCGCT |
| | AACCAGTTTAATTCCGCAATTGGAAAGATCCAAGATTCACT |
| | CAGCTCAACCGCCTCTGCACTCGGAAAGCTGCAGGACGTG |
| | GTCAACCAGAATGCTCAGGCCCTGAACACACTCGTCAAGC |
| | AGCTGTCCTCTAACTTTGGCGCTATCAGCTCCGTTCTGAAC |
| | GACATTCTGAGCCGCCTGGATCCCCCAGAGGCTGAAGTCCA |
| | GATTGACCGCCTGATTACCGGCCGGCTGCAGTCTCTGCAAA |
| | CATACGTGACCCAGCAGCTGATCAGAGCAGCCGAGATCCG |
| | GGCATCCGCAAATCTGGCAGCAATCAAGATGAGCGAATGC |
| | GTGCTGGGCCAGTCCAAGCGGGTGGACTTTTGTGGCAAGG |
| | GCTACCACCTGATGAGCTTCCCCCAGAGCGCCCCACATGGC |
| | GTTGTTTTTCTGCACGTGACCTATGTCCCTGCTCAGGAAAA |
| | GAACTTTACAACTGCTCCTGCTATCTGCCATGACGGCAAGG |
| | CCCACTTCCCACGGGAGGGAGTGTTTGTGTCCAATGGCACA |
| | CACTGGTTCGTGACCCAGAGGAACTTCTATGAACCCCAGAT |
| | CATCACCACTGACAATACCTTCGTGTCTGGAAATTGCGACG |
| | TCGTGATCGGCATCGTTAACAACACCGTGTACGACCCTCTC |
| | CAGCCAGAGCTGGACTCCTTTAAGGAGGAACTGGATAAGT |
| | ATTTTAAGAACCACACAAGCCCAGATGTGGATCTCGGGGA |
| | CATCTCCGGAATTAACGCCTCCTTCGTGAATATCCAGAAGG |
| | AGATTGACCGCCTAAATGAAGTTGCCAAGAACCTCAATGA |
| | GTCTCTGATTGATCTGCAGGAACTGGGCAAGTATGAGCAGT |
| | ATATCAAATGGCCCTGGTACATTTGGCTGGGGTTTATCGCC |
| | GGACTGATTGCCATCGTCATGGTGACCATCATGCTGTGTTG |
| | CATGACCTCCTGTTGTTCCTGTCTGAAGGGCTGCTGTAGTT |
| | GCGGCTCTTGCTGTAAATTCGACGAAGATGATAGCGAGCCC |
| | GTGCTGAAGGGCGTGAAGCTGCATTATACCTGA |
| SARS-CoV-2 S protein | (SEQ ID NO: 171) |
| containing mutated to | MFVFLVLLPLVSSQCVNFTNRTQLPSAYTNSFTRGVYYPDKV |
| remove a furin cleavage | FRSSVLHSTQDLFLPFFSNVTWFHAIHVSGTNGTKRFDNPVLP |
| site and to replace | FNDGVYFASTEKSNIIRGWIFGTTLDSKTQSLLIVNNATNVVIK |
| residues 986 and 987 | VCEFQFCNYPFLGVYYHKNNKSWMESEFRVYSSANNCTFEY |
| with proline and which | VSQPFLMDLEGKQGNFKNLSEFVFKNIDGYFKIYSKHTPINLV |
| contains the L18F, | RDLPQGFSALEPLVDLPIGINITRFQTLLALHRSYLTPGDSSSG |
| T20N, P26S, D138Y, | WTAGAAAYYVGYLQPRTFLLKYNENGTITDAVDCALDPLSET |
| R190S, K417T, E484K, | KCTLKSFTVEKGIYQTSNFRVQPTESIVRFPNITNLCPFGEVEN |
| N501Y, D614G, H655Y, | ATRFASVYAWNRKRISNCVADYSVLYNSASFSTFKCYGVSPT |
| T10271 and V1176F | KLNDLCFTNVYADSFVIRGDEVRQIAPGQTGTIADYNYKLPD |
| mutations | DFTGCVIAWNSNNLDSKVGGNYNYLYRLFRKSNLKPFERDIS |
| | TEIYQAGSTPCNGVKGFNCYFPLQSYGFQPTYGVGYQPYRVV |
| | VLSFELLHAPATVCGPKKSTNLVKNKCVNFNFNGLTGTGVLT |
| | ESNKKFLPFQQFGRDIADTTDAVRDPQTLEILDITPCSFGGVSV |
| | ITPGTNTSNQVAVLYQGVNCTEVPVAIHADQLTPTWRVYSTG |
| | SNVFQTRAGCLIGAEYVNNSYECDIPIGAGICASYQTQTNSPGS |
| | ASSVASQSIIAYTMSLGAENSVAYSNNSIAIPTNFTISVTTEILPV |
| | SMTKTSVDCTMYICGDSTECSNLLLQYGSFCTQLNRALTGIAV |
| | EQDKNTQEVFAQVKQIYKTPPIKDFGGFNFSQILPDPSKPSKRS |
| | FIEDLLFNKVTLADAGFIKQYGDCLGDIAARDLICAQKENGLT |
| | VLPPLLTDEMIAQYTSALLAGTITSGWTFGAGAALQIPFAMQ |
| | MAYRFNGIGVTQNVLYENQKLIANQFNSAIGKIQDSLSSTASA |
| | LGKLQDVVNQNAQALNTLVKQLSSNFGAISSVLNDILSRLDPP |
| | EAEVQIDRLITGRLQSLQTYVTQQLIRAAEIRASANLAAIKMSE |
| | CVLGQSKRVDFCGKGYHLMSFPQSAPHGVVFLHVTYVPAQE |
| | KNFTTAPAICHDGKAHFPREGVFVSNGTHWFVTQRNFYEPQII |
| | TTDNTFVSGNCDVVIGIVNNTVYDPLQPELDSFKEELDKYFKN |
| | HTSPDVDLGDISGINASFVNIQKEIDRLNEVAKNLNESLIDLQE |
| | LGKYEQYIKWPWYIWLGFIAGLIAIVMVTIMLCCMTSCCSCLK |
| | GCCSCGSCCKFDEDDSEPVLKGVKLHYT |

### Peptide Fusions

The inventors have identified regions in the SARS-COV-2 S protein which are likely to be highly antigenic. These include residues 815-833 (FP), 820-846 (D1) 1078-1111 (D2) and residues 815-846 (F1/D1). The sequences for these antigenic fragments in the full-length SARS-CoV-2 protein with the amino acid sequence of SEQ ID NO: 1 are SFIEDLLFNKVTLADAGF (SEQ ID NO: 21), LLFNKVTLADAGFIKQYGDCLGDIAA (SEQ ID NO: 22), PAICHDGKAHFPREGVFVSNGTHWFVTQRNFYE (SEQ ID NO: 23), and GGFNFSQILPDPSKPSKRSFIEDLLFNKVTLA (SEQ ID NO: 24), respectively. The antigenic regions can be arranged in different orders to form a variety of fusion peptides that are likely to be highly antigenic and therefore are expected to induce a strong immunogenic response. The domains can be linked by a linker sequence, e.g., GGGGS. Alternatively, given the similarity in their amino acid sequences, the FP and DI regions can be overlapped to produce a single immunogenic motif:

(SEQ ID NO: 99)
SFIEDLLFNKVTLADAGFIKQYGDCLGDIAA (FP/D1),

with the overlap sequence underlined.

An exemplary peptide fusion may have the following domains:
D1-linker-FP-linker-D2-linker-D1 (Fusion peptide A)
FP/D1-linker-FP/D1-linker-FP/D1 (Fusion peptide B)

Accordingly, the invention provides optimized nucleotide sequences that encode fusion peptides comprising antigenic regions of the SARS-COV-2 S protein. In one embodiment, an optimized nucleotide sequence encodes an amino acid sequence comprising Fusion peptide A. For example, the optimized nucleotide sequence can encode the amino acid sequence of SEQ ID NO: 25. In a particular embodiment, the optimized nucleotide sequence has the sequence of SEQ ID NO: 26. In another embodiment, the optimized nucleotide sequence encodes an amino acid sequence comprising Fusion peptide B. For example, the optimized nucleotide sequence can encode an amino acid sequence of SEQ ID NO: 27. In a particular embodiment, the optimized nucleotide sequence has the sequence of SEQ ID NO: 28.

In certain embodiments, the fusion peptide may be operably linked to an N terminal signal sequence, such as SEQ ID NO: 7. For example, an optimized nucleotide sequence may encode an amino acid sequence comprising Fusion peptide A operably linked with an N terminal signal sequence. The optimized nucleotide sequence can encode the amino acid sequence of SEQ ID NO: 51. In a particular embodiment, the optimized nucleotide sequence has the sequence of SEQ ID NO: 52. Alternatively, the optimized nucleotide sequence may encode an amino acid sequence comprising Fusion peptide B operably linked with an N terminal signal sequence. The optimized nucleotide sequence can encode the amino acid sequence of SEQ ID NO: 53. In a particular embodiment, the optimized nucleotide sequence has the sequence of SEQ ID NO: 54.

Additionally, the fusion peptides can be operably linked with a C-terminal Fc domain, typically in addition to an N terminal signal sequence. For example, an optimized nucleotide sequence may encode an amino acid sequence comprising Fusion peptide A operably linked with a C terminal Fc domain (e.g., SEQ ID NO: 18) and an N terminal signal sequence (e.g., SEQ ID NO: 7). The optimized nucleotide sequence can encode the amino acid sequence of SEQ ID NO: 55. In a particular embodiment, the optimized nucleotide sequence has the sequence of SEQ ID NO: 56. Alternatively, the optimized nucleotide sequence may encode an amino acid sequence comprising Fusion peptide B operably linked with a C terminal Fc domain (e.g., SEQ ID NO: 18) and an N terminal signal sequence (e.g., SEQ ID NO: 7). The optimized nucleotide sequence can encode the amino acid sequence of SEQ ID NO: 57. In a particular embodiment, the optimized nucleotide sequence has the sequence of SEQ ID NO: 58.

In some embodiments, the fusion peptides can be operably linked with a C terminal Fc domain which has been altered to improve circulation half-life of the resulting fusion protein. In particular embodiment, the Fc domain with improve circulation half-life has the amino acid sequence of SEQ ID NO: 100, SEQ ID NO: 101, SEQ ID NO: 102 or SEQ ID NO: 103. Accordingly, the invention also provides an optimized nucleotide sequence that encodes Fusion peptide A or Fusion peptide B, operably linked with an N-terminal signal peptide and a C-terminal Fc domain having the amino acid sequence of SEQ ID NO: 100, SEQ ID NO: 101, SEQ ID NO: 102 or SEQ ID NO: 103. The signal peptide can have the amino acid sequence of SEQ ID NO:7.

### Exemplary Optimized Nucleotide Sequences Encoding a Fusion Peptide

An optimized nucleotide sequence according to the present invention may encode one or more antigenic regions of a SARS-COV-2 S protein in the form of a fusion peptide. In one embodiment, the invention provides a nucleic acid comprising an optimized nucleotide sequence encoding one or more antigenic regions of the SARS-COV-2 S protein in the form of a fusion peptide. In some embodiments, the nucleic acid is an mRNA comprising an optimized nucleotide sequence encoding one or more antigenic regions of the SARS-COV-2 S protein in the form of a fusion peptide. In some embodiments, a suitable mRNA sequence comprises a nucleotide sequence encoding one or more antigenic regions of the SARS-COV-2 S protein in the form of a fusion peptide optimized for efficient expression in human cells. Exemplary optimized nucleotide sequences encoding antigenic regions of the SARS-COV-2 S protein in the form of a fusion peptide produced with the process for generating optimized nucleotide sequences in accordance with the invention and the corresponding amino acid sequence are shown in Table 2. Bold residues indicate those amino acids which have been mutated compared to a naturally occurring SARS-COV-2 S protein, underlined residues represent a signal peptide and the residues in italics indicate the presence of an Fc region.

TABLE 2
Exemplary fusion peptides.

| | |
|---|---|
| Optimized nucleotide | (SEQ ID NO: 25) |
| sequence encoding Fusion | ATGCTGCTGTTTAACAAAGTGACTCTGGCAGACGCAG |
| peptide A | GCTTTATCAAGCAGTACGGAGACTGTCTCGGGGACAT |
| | TGCAGCCGGCGGCGGAGGCTCATCTTTCATTGAGGAC |
| | CTGCTGTTCAACAAGGTCACTCTGGCAGATGCCGGAT |
| | TCGGAGGAGGGGGATCTCCAGCTATCTGCCATGACGG |
| | AAAGGCTCATTTTCCTCGGGAGGGTGTGTTTGTGTCCA |
| | ACGGAACCCATTGGTTCGTCACACAGCGCAACTTCTA |
| | TGAAGGAGGGGGGGGCTCCAGCTTCATCGAGGACCTG |
| | CTCTTTAACAAAGTGACCCTGGCCGATGCTGGATTTG |
| | GGGGAGGGGGATCCCTGCTGTTCAACAAAGTTACACT |
| | GGCCGACGCAGGCTTCATCAAACAGTACGGCGATTGT |
| | TTAGGGGACATCGCCGCTGGCGGCGGAGGATCACCTA |
| | AGTCCTGCGACAAAACCCATACATGTCCACCATGCCC |
| | AGCTCCTGAACTGCTCGGCGGGCCTAGTGTTTTCCTCT |
| | TCCCTCCTAAGCCCAAGGATACCCTCATGATCTCTCGC |
| | ACACCAGAAGTGACCTGCGTGGTCGTGGATGTCTCTC |
| | ACGAGGATCCTGAAGTGAAGTTTAACTGGTATGTCGA |
| | CGGAGTGGAAGTGCACAACGCCAAGACAAAGCCAAG |
| | AGAAGAACAATACAATTCTACTTATAGGGTGGTGTCT |
| | GTGCTGACAGTGCTGCACCAGGATTGGCTGAATGGAA |
| | AAGAATATAAGTGTAAGGTCTCTAACAAGGCCCTGCC |
| | CGCTCCAATTGAGAAGACAATTTCCAAGGCCAAGGGG |
| | CAGCCTCGGGAACCTCAGGTGTACACACTGCCCCCAT |
| | CCAGGGATGAACTGACTAAAAATCAGGTGTCTCTGAC |
| | ATGCCTGGTGAAAGGGTTTTATCCAAGTGACATTGCT |
| | GTGGAGTGGGAGTCTAATGGGCAGCCTGAAAATAACT |
| | ACAAGACCACACCACCAGTGCTCGATAGCGACGGGTC |
| | TTTCTTTCTGTATTCTAAACTGACCGTGGATAAATCTC |
| | GGTGGCAGCAGGGAAACGTGTTTTCTTGCTCAGTGAT |
| | GCACGAAGCTCTGCACAATCACTATACACAGAAATCC |
| | CTGTCCCTGTCTCCAGGCAAATAA |
| Fusion peptide A | (SEQ ID NO: 26) |
| | MLLFNKVTLADAGFIKQYGDCLGDIAAGGGGSSFIEDLL |
| | FNKVTLADAGFGGGGSPAICHDGKAHFPREGVFVSNGT |
| | HWFVTQRNFYEGGGGSSFIEDLLFNKVTLADAGFGGGG |
| | SLLFNKVTLADAGFIKQYGDCLGDIAA |
| Optimized nucleotide | (SEQ ID NO: 27) |
| sequence encoding Fusion | ATGTCCTTCATTGAGGACCTGCTGTTTAATAAGGTGAC |
| peptide B | CCTGGCCGACGCTGGGTTCATCAAACAGTATGGAGAT |
| | TGTCTGGGAGATATTGCAGCAGGCGGGGGCGGCAGC |
| | AGCTTTATTGAGGACCTCCTGTTCAACAAGGTGACCC |
| | TTGCCGACGCAGGGTTTATTAAGCAGTATGGCGACTG |
| | TCTGGGAGACATTGCAGCCGGCGGCGGCGGGTCTTCT |
| | TTTATCGAGGACCTGCTGTTCAACAAGGTGACACTGG |
| | CCGACGCAGGCTTTATTAAGCAGTACGGGGACTGCCT |
| | GGGAGACATTGCCGCCTGA |
| Fusion peptide B | (SEQ ID NO: 28) |
| | MSFIEDLLFNKVTLADAGFIKQYGDCLGDIAAGGGGSSFI |
| | EDLLFNKVTLADAGFIKQYGDCLGDIAAGGGGSSFIEDL |
| | LFNKVTLADAGFIKQYGDCLGDIAA |
| Optimized nucleotide | (SEQ ID NO: 52) |
| sequence encoding Fusion | ATGTTCGTGTTCCTGGTGCTGCTGCCACTGGTTTCCTC |
| peptide A with a signal | CCAGTGTCTGCTGTTTAACAAGGTTACACTGGCAGAC |
| peptide | GCCGGCTTCATCAAGCAGTATGGGGACTGTCTGGGCG |
| | ATATCGCCGCTGGCGGCGGAGGATCTAGCTTCATTGA |
| | GGACCTGCTGTTCAACAAAGTGACTCTGGCTGACGCC |
| | GGATTTGGCGGAGGAGGGTCTCCTGCCATTTGTCATG |
| | ACGGGAAGGCTCATTTCCCTAGGGAGGGGGTTTTTGT |
| | CTCCAATGGAACTCACTGGTTCGTGACCCAAAGAAAC |
| | TTCTATGAGGGAGGTGGCGGATCCTCTTTTATCGAGG |
| | ACCTGCTGTTTAACAAGGTCACTCTGGCCGATGCAGG |
| | CTTCGGAGGAGGAGGGTCTCTGCTGTTCAACAAAGTT |
| | ACTCTGGCAGATGCTGGGTTCATTAAGCAGTACGGCG |
| | ACTGTCTGGGCGATATTGCCGCCTGA |
| Fusion peptide A with a | (SEQ ID NO: 51) |
| signal peptide | MFVFLVLLPLVSSQCLLFNKVTLADAGFIKQYGDCLGDI |
| | AAGGGGSSFIEDLLFNKVTLADAGFGGGGSPAICHDGKA |
| | HFPREGVFVSNGTHWFVTQRNFYEGGGGSSFIEDLLENK |
| | VTLADAGFGGGGSLLFNKVTLADAGFIKQYGDCLGDIA |
| | A |
| Optimized nucleotide | (SEQ ID NO: 54) |
| sequence encoding Fusion | ATGTTCGTGTTCCTGGTCCTGCTACCCCTGGTGTCCTC |
| peptide B with a signal | TCAGTGCTCCTTCATTGAGGACCTGCTGTTTAATAAGG |
| peptide | TGACCCTGGCCGACGCTGGGTTCATCAAACAGTATGG |
| | AGATTGTCTGGGAGATATTGCAGCAGGCGGGGGCGGC |
| | AGCAGCTTTATTGAGGACCTCCTGTTCAACAAGGTGA |
| | CCCTTGCCGACGCAGGGTTTATTAAGCAGTATGGCGA |
| | CTGTCTGGGAGACATTGCAGCCGGCGGCGGCGGGTCT |
| | TCTTTTATCGAGGACCTGCTGTTCAACAAGGTGACACT |
| | GGCCGACGCAGGCTTTATTAAGCAGTACGGGGACTGC |
| | CTGGGAGACATTGCCGCCTGA |
| Fusion peptide B with a | (SEQ ID NO: 53) |
| signal peptide | MFVFLVLLPLVSSQCSFIEDLLFNKVTLADAGFIKQYGD |
| | CLGDIAAGGGGSSFIEDLLFNKVTLADAGFIKQYGDCLG |
| | DIAAGGGGSSFIEDLLFNKVTLADAGFIKQYGDCLGDIA |
| | A |
| Optimized nucleotide | (SEQ ID NO: 56) |
| sequence encoding Fusion | ATGTTTGTGTTCCTCGTTCTGCTGCCTCTGGTGAGCTC |
| peptide A with a signal | CCAGTGTCTGCTGTTTAACAAAGTGACTCTGGCAGAC |
| peptide and an Fc region | GCAGGCTTTATCAAGCAGTACGGAGACTGTCTCGGGG |
| | ACATTGCAGCCGGCGGCGGAGGCTCATCTTTCATTGA |
| | GGACCTGCTGTTCAACAAGGTCACTCTGGCAGATGCC |
| | GGATTCGGAGGAGGGGGATCTCCAGCTATCTGCCATG |
| | ACGGAAAGGCTCATTTTCCTCGGGAGGGTGTGTTTGT |
| | GTCCAACGGAACCCATTGGTTCGTCACACAGCGCAAC |
| | TTCTATGAAGGAGGGGGGGGCTCCAGCTTCATCGAGG |
| | ACCTGCTCTTTAACAAAGTGACCCTGGCCGATGCTGG |
| | ATTTGGGGGAGGGGGATCCCTGCTGTTCAACAAAGTT |
| | ACACTGGCCGACGCAGGCTTCATCAAACAGTACGGCG |
| | ATTGTTTAGGGGACATCGCCGCTGGCGGCGGAGGATC |
| | ACCTAAGTCCTGCGACAAAACCCATACATGTCCACCA |
| | TGCCCAGCTCCTGAACTGCTCGGCGGGCCTAGTGTTTT |
| | CCTCTTCCCTCCTAAGCCCAAGGATACCCTCATGATCT |
| | CTCGCACACCAGAAGTGACCTGCGTGGTCGTGGATGT |
| | CTCTCACGAGGATCCTGAAGTGAAGTTTAACTGGTAT |
| | GTCGACGGAGTGGAAGTGCACAACGCCAAGACAAAG |
| | CCAAGAGAAGAACAATACAATTCTACTTATAGGGTGG |
| | TGTCTGTGCTGACAGTGCTGCACCAGGATTGGCTGAA |
| | TGGAAAAGAATATAAGTGTAAGGTCTCTAACAAGGCC |
| | CTGCCCGCTCCAATTGAGAAGACAATTTCCAAGGCCA |
| | AGGGGCAGCCTCGGGAACCTCAGGTGTACACACTGCC |
| | CCCATCCAGGGATGAACTGACTAAAAATCAGGTGTCT |
| | CTGACATGCCTGGTGAAAGGGTTTTATCCAAGTGACA |
| | TTGCTGTGGAGTGGGAGTCTAATGGGCAGCCTGAAAA |
| | TAACTACAAGACCACACCACCAGTGCTCGATAGCGAC |
| | GGGTCTTTCTTTCTGTATTCTAAACTGACCGTGGATAA |
| | ATCTCGGTGGCAGCAGGGAAACGTGTTTTCTTGCTCA |
| | GTGATGCACGAAGCTCTGCACAATCACTATACACAGA |
| | AATCCCTGTCCCTGTCTCCAGGCAAATAA |
| Fusion peptide A with a | (SEQ ID NO: 55) |
| signal peptide and an Fc | MFVFLVLLPLVSSQCLLFNKVTLADAGFIKQYGDCLGDI |
| region | AAGGGGSSFIEDLLFNKVTLADAGFGGGGSPAICHDGKA |
| | HFPREGVFVSNGTHWFVTQRNFYEGGGGSSFIEDLLENK |
| | VTLADAGFGGGGSLLFNKVTLADAGFIKQYGDCLGDIA |
| | AGGGGSPKSCDKTHTCPPCPAPELLGGPSVFLFPPKPKDT |
| | LMISRTPEVTCVVVDVSHEDPEVKFNWYVDGVEVHNAKTK |
| | PREEQYNSTYRVVSVLTVLHQDWLNGKEYKCKVSNKALPAP |
| | IEKTISKAKGQPREPQVYTLPPSRDELTKNQVSLTCLVKGFY |
| | PSDIAVEWESNGQPENNYKTTPPVLDSDGSFFLYSKLTVDKS |
| | RWQQGNVFSCSVMHEALHNHYTQKSLSLSPGK |
| Optimized nucleotide | (SEQ ID NO: 58) |
| sequence encoding Fusion | ATGTTCGTGTTCCTGGTCCTGCTGCCTCTGGTGTCCTC |
| peptide B with a signal | TCAGTGCAGCTTCATCGAGGACCTGCTCTTTAACAAG |
| peptide and an Fc region | GTGACTCTCGCAGATGCTGGCTTCATCAAGCAGTACG |
| | GAGACTGCCTTGGAGACATCGCTGCAGGCGGAGGGG |
| | GCAGCAGTTTCATCGAGGACCTGCTGTTTAACAAGGT |
| | GACCCTGGCCGACGCCGGGTTCATTAAGCAATACGGC |
| | GATTGTCTGGGAGACATCGCAGCTGGGGGAGGGGGG |
| | AGCTCTTTTATTGAGGACCTGCTGTTCAACAAGGTGA |
| | CTCTGGCCGACGCAGGGTTCATCAAACAGTATGGGGA |
| | CTGTCTGGGAGATATCGCAGCCGGGGGAGGAGGCTCC |
| | CCTAAGTCCTGCGACAAAACCCATACATGTCCACCAT |
| | GCCCAGCTCCTGAACTGCTCGGCGGGCCTAGTGTTTTC |
| | CTCTTCCCTCCTAAGCCCAAGGATACCCTCATGATCTC |
| | TCGCACACCAGAAGTGACCTGCGTGGTCGTGGATGTC |
| | TCTCACGAGGATCCTGAAGTGAAGTTTAACTGGTATG |
| | TCGACGGAGTGGAAGTGCACAACGCCAAGACAAAGC |
| | CAAGAGAAGAACAATACAATTCTACTTATAGGGTGGT |
| | GTCTGTGCTGACAGTGCTGCACCAGGATTGGCTGAAT |
| | GGAAAAGAATATAAGTGTAAGGTCTCTAACAAGGCCC |
| | TGCCCGCTCCAATTGAGAAGACAATTTCCAAGGCCAA |
| | GGGGCAGCCTCGGGAACCTCAGGTGTACACACTGCCC |
| | CCATCCAGGGATGAACTGACTAAAAATCAGGTGTCTC |
| | TGACATGCCTGGTGAAAGGGTTTTATCCAAGTGACAT |
| | TGCTGTGGAGTGGGAGTCTAATGGGCAGCCTGAAAAT |
| | AACTACAAGACCACACCACCAGTGCTCGATAGCGACG |
| | GGTCTTTCTTTCTGTATTCTAAACTGACCGTGGATAAA |
| | TCTCGGTGGCAGCAGGGAAACGTGTTTTCTTGCTCAG |
| | TGATGCACGAAGCTCTGCACAATCACTATACACAGAA |
| | ATCCCTGTCCCTGTCTCCAGGCAAATAA |
| Fusion peptide B with a | (SEQ ID NO: 57) |
| signal peptide and an Fc | MFVFLVLLPLVSSQCSFIEDLLFNKVTLADAGFIKQYGD |
| region | CLGDIAAGGGGSSFIEDLLFNKVTLADAGFIKQYGDCLG |
| | DIAAGGGGSSFIEDLLFNKVTLADAGFIKQYGDCLGDIA |
| | AGGGGSPKSCDKTHTCPPCPAPELLGGPSVFLFPPKPKDT |
| | LMISRTPEVTCVVVDVSHEDPEVKFNWYVDGVEVHNAKTK |
| | PREEQYNSTYRVVSVLTVLHQDWLNGKEYKCKVSNKALPAP |
| | IEKTISKAKGQPREPQVYTLPPSRDELTKNQVSLTCLVKGFY |
| | PSDIAVEWESNGQPENNYKTTPPVLDSDGSFFLYSKLTVDKS |
| | RWQQGNVFSCSVMHEALHNHYTQKSLSLSPGK |

### Other Essential Structural Proteins

Based on their homology to proteins in related β-coronaviruses, the M, N and E proteins of SARS-COV-2 are considered to play important roles in forming the structure of the virus particle. The M protein is believed to be the most abundant structural protein in the virion. It is 222 amino acids in length with 3 transmembrane domains. It has been proposed that the M protein gives the virus particle its shape. The M protein is suggested to exist as a dimer in the virion where it may adopt two different conformations allowing it to promote membrane curvature and bind to the nucleocapsid.

The 419 amino acid long N protein likely forms the nucleocapsid. It is composed of two separate domains, which are both capable of binding RNA in vitro using different mechanisms. The N protein binds the viral genome in a beads-on-a-string type conformation and can also bind to nsp3, a key component of the viral replicase complex, and the M protein.

The E protein is 77 amino acids in length and is believed to be present only in small quantities within the virus particle. One of the E protein's proposed functions is to facilitate the assembly and release of the virus. The amino acid sequence for the M, N and E proteins of SARS-CoV-2 are shown in Table 3 below.

While memory CD8+ T cells have broad reactivity against many SARS-COV-2 proteins, including ORF1ab, S, N, M, and ORF3a, most of the epitopes are located in ORF1ab and the highest density of epitopes is located in the N protein (Ferretti et al. (2020) https://doi.org/10.1101/2020.07.24.20161653). ORF1ab is encoded by residues 266 . . . 13555 of the NC 045512.2 SARS-COV-2 genome. The ORF1ab and N proteins of SARS-COV-2 may therefore be useful for inducing a T cell response.

TABLE 3
SARS-CoV-2 M, E and N proteins

| | |
|---|---|
| Nucleotide sequence of | (SEQ ID NO: 59) |
| SARS-CoV-2 M protein | ATGGCAGACAACGGTACTATTACCGTTGAGGAGCTTA |
| NC_004718.3 SARS-CoV-2 | AACAACTCCTGGAACAATGGAACCTAGTAATAGGTTT |
| genome | CCTATTCCTAGCCTGGATTATGTTACTACAATTTGCCT |
| Range 26398..27063 | ATTCTAATCGGAACAGGTTTTTGTACATAATAAAGCTT |
| | GTTTTCCTCTGGCTCTTGTGGCCAGTAACACTTGCTTG |
| | TTTTGTGCTTGCTGCTGTCTACAGAATTAATTGGGTGA |
| | CTGGCGGGATTGCGATTGCAATGGCTTGTATTGTAGG |
| | CTTGATGTGGCTTAGCTACTTCGTTGCTTCCTTCAGGC |
| | TGTTTGCTCGTACCCGCTCAATGTGGTCATTCAACCCA |
| | GAAACAAACATTCTTCTCAATGTGCCTCTCCGGGGGA |
| | CAATTGTGACCAGACCGCTCATGGAAAGTGAACTTGT |
| | CATTGGTGCTGTGATCATTCGTGGTCACTTGCGAATGG |
| | CCGGACACTCCCTAGGGCGCTGTGACATTAAGGACCT |
| | GCCAAAAGAGATCACTGTGGCTACATCACGAACGCTT |
| | TCTTATTACAAATTAGGAGCGTCGCAGCGTGTAGGCA |
| | CTGATTCAGGTTTTGCTGCATACAACCGCTACCGTATT |
| | GGAAACTATAAATTAAATACAGACCACGCCGGTAGCA |
| | ACGACAATATTGCTTTGCTAGTACAGTAA |
| SARS-CoV-2 M protein | (SEQ ID NO: 60) |
| sequence | MADSNGTITVEELKKLLEQWNLVIGFLFLTWICLLQFAY |
| Accession number | ANRNRFLYIIKLIFLWLLWPVTLACFVLAAVYRINWITG |
| QII57163 | GIAIAMACLVGLMWLSYFIASFRLFARTRSMWSFNPETN |
| | ILLNVPLHGTILTRPLLESELVIGAVILRGHLRIAGHHLGR |
| | CDIKDLPKEITVATSRTLSYYKLGASQRVAGDSGFAAYS |
| | RYRIGNYKLNTDHSSSSDNIALLVQ |
| Nucleotide sequence of | (SEQ ID NO: 61) |
| SARS-CoV-2 E protein | ATGTACTCATTCGTTTCGGAAGAAACAGGTACGTTAA |
| NC_004718.3 SARS-CoV-2 | TAGTTAATAGCGTACTTCTTTTTCTTGCTTTCGTGGTAT |
| genome | TCTTGCTAGTCACACTAGCCATCCTTACTGCGCTTCGA |
| Range 26117..26347 | TTGTGTGCGTACTGCTGCAATATTGTTAACGTGAGTTT |
| | AGTAAAACCAACGGTTTACGTCTACTCGCGTGTTAAA |
| | AATCTGAACTCTTCTGAAGGAGTTCCTGATCTTCTGGT |
| | CTAA |
| SARS-CoV-2 E protein | (SEQ ID NO: 62) |
| sequence | MYSFVSEETGTLIVNSVLLFLAFVVFLLVTLAILTALRLC |
| Accession number P59637.1 | AYCCNIVNVSLVKPSFYVYSRVKNLNSSRVPDLLV |
| Nucleotide sequence of | (SEQ ID NO: 63) |
| SARS-CoV-2 N protein | ATGTCTGATAATGGACCCCAAAATCAGCGAAATGCAC |
| NC_045512.2 SARS-CoV-2 | CCCGCATTACGTTTGGTGGACCCTCAGATTCAACTGG |
| genome | CAGTAACCAGAATGGAGAACGCAGTGGGGCGCGATC |
| range 28274..29533 | AAAACAACGTCGGCCCCAAGGTTTACCCAATAATACT |
| | GCGTCTTGGTTCACCGCTCTCACTCAACATGGCAAGG |
| | AAGACCTTAAATTCCCTCGAGGACAAGGCGTTCCAAT |
| | TAACACCAATAGCAGTCCAGATGACCAAATTGGCTAC |
| | TACCGAAGAGCTACCAGACGAATTCGTGGTGGTGACG |
| | GTAAAATGAAAGATCTCAGTCCAAGATGGTATTTCTA |
| | CTACCTAGGAACTGGGCCAGAAGCTGGACTTCCCTAT |
| | GGTGCTAACAAAGACGGCATCATATGGGTTGCAACTG |
| | AGGGAGCCTTGAATACACCAAAAGATCACATTGGCAC |
| | CCGCAATCCTGCTAACAATGCTGCAATCGTGCTACAA |
| | CTTCCTCAAGGAACAACATTGCCAAAAGGCTTCTACG |
| | CAGAAGGGAGCAGAGGCGGCAGTCAAGCCTCTTCTCG |
| | TTCCTCATCACGTAGTCGCAACAGTTCAAGAAATTCA |
| | ACTCCAGGCAGCAGTAGGGGAACTTCTCCTGCTAGAA |
| | TGGCTGGCAATGGCGGTGATGCTGCTCTTGCTTTGCTG |
| | CTGCTTGACAGATTGAACCAGCTTGAGAGCAAAATGT |
| | CTGGTAAAGGCCAACAACAACAAGGCCAAACTGTCA |
| | CTAAGAAATCTGCTGCTGAGGCTTCTAAGAAGCCTCG |
| | GCAAAAACGTACTGCCACTAAAGCATACAATGTAACA |
| | CAAGCTTTCGGCAGACGTGGTCCAGAACAAACCCAAG |
| | GAAATTTTGGGGACCAGGAACTAATCAGACAAGGAA |
| | CTGATTACAAACATTGGCCGCAAATTGCACAATTTGC |
| | CCCCAGCGCTTCAGCGTTCTTCGGAATGTCGCGCATTG |
| | GCATGGAAGTCACACCTTCGGGAACGTGGTTGACCTA |
| | CACAGGTGCCATCAAATTGGATGACAAAGATCCAAAT |
| | TTCAAAGATCAAGTCATTTTGCTGAATAAGCATATTG |
| | ACGCATACAAAACATTCCCACCAACAGAGCCTAAAAA |
| | GGACAAAAAGAAGAAGGCTGATGAAACTCAAGCCTT |
| | ACCGCAGAGACAGAAGAAACAGCAAACTGTGACTCT |
| | TCTTCCTGCTGCAGATTTGGATGATTTCTCCAAACAAT |
| | TGCAACAATCCATGAGCAGTGCTGACTCAACTCAGGC |
| | CTAA |
| SARS-CoV-2 N protein | (SEQ ID NO: 64) |
| sequence | MSDNGPQNQRNAPRITFGGPSDSTGSNQNGERSGARSK |
| Accession number | QRRPQGLPNNTASWFTALTQHGKEDLKFPRGQGVPINT |
| QIS29990.1 | NSSPDDQIGYYRRATRRIRGGDGKMKDLSPRWYFYYLG |
| | TGPEAGLPYGANKDGIIWVATEGALNTPKDHIGTRNPAN |
| | NAAIVLQLPQGTTLPKGFYAEGSRGGSQASSRSSSRSRNS |
| | SRNLTPGSSRGTSPARMAGNGGDAALALLLLDRLNQLE |
| | SKMSGKGQQQQGQTVTKKSAAEASKKPRQKRTATKAY |
| | NVTQAFGRRGPEQTQGNFGDQELIRQGTDYKHWPQIAQ |
| | FAPSASAFFGMSRIGMEVTPSGTWLTYTGAIKLDDKDPN |
| | FKDQVILLNKHIDAYKTFPPTEPKKDKKKKADETQALPQ |
| | RQKKQQTVTLLPAADLDDFSKQLQQSMSSADSTQA |

An optimized nucleotide sequence according to the present invention may encode a SARS-COV-2 E protein or an antigenic fragment thereof. In one embodiment, the invention provides a nucleic acid comprising an optimized nucleotide sequence encoding a SARS-COV-2 E protein or an antigenic fragment thereof. In some embodiments, the nucleic acid is an mRNA comprising an optimized nucleotide sequence encoding a SARS-COV-2 E protein or an antigenic fragment thereof. In some embodiments, a suitable mRNA sequence comprises a nucleotide sequence encoding a SARS-COV-2 small envelope protein or an antigenic fragment thereof optimized for efficient expression in human cells.

An optimized nucleotide sequence according to the present invention may encode a SARS-COV-2 M protein or an antigenic fragment thereof. In one embodiment, the invention provides a nucleic acid comprising an optimized nucleotide sequence encoding a SARS-COV-2 M protein or an antigenic fragment thereof. In some embodiments, the nucleic acid is an mRNA comprising an optimized nucleotide sequence encoding a SARS-COV-2 M protein or an antigenic fragment thereof. In some embodiments, a suitable mRNA sequence comprises a nucleotide sequence encoding a SARS-COV-2 M protein or an antigenic fragment thereof optimized for efficient expression in human cells. An optimized nucleotide sequence according to the present invention may encode a SARS-COV-2 N protein or an antigenic fragment thereof. In one embodiment, the invention provides a nucleic acid comprising an optimized nucleotide sequence encoding a SARS-COV-2 N protein or an antigenic fragment thereof. In some embodiments, the nucleic acid is an mRNA comprising an optimized nucleotide sequence encoding a SARS-COV-2 N protein or an antigenic fragment thereof. In some embodiments, a suitable mRNA sequence comprises a nucleotide sequence encoding a SARS-COV-2 N protein or an antigenic fragment thereof optimized for efficient expression in human cells.

An optimized nucleotide sequence according to the present invention may encode a SARS-COV-2 ORF1ab protein or an antigenic fragment thereof. In one embodiment, the invention provides a nucleic acid comprising an optimized nucleotide sequence encoding a SARS-COV-2 ORF1ab protein or an antigenic fragment thereof. In some embodiments, the nucleic acid is an mRNA comprising an optimized nucleotide sequence encoding a SARS-COV-2 ORF1ab protein or an antigenic fragment thereof. In some embodiments, a suitable mRNA sequence comprises a nucleotide sequence encoding a SARS-COV-2 ORF1ab protein or an antigenic fragment thereof optimized for efficient expression in human cells.

In some embodiments, a first nucleic acid comprising an optimized nucleotide sequence encoding a SARS-COV-2 S protein or an antigenic fragment thereof is combined with a second nucleic acid comprising an optimized nucleotide sequence encoding a SARS-COV-2 M protein or an antigenic fragment thereof. In some embodiments, a first nucleic acid comprising an optimized nucleotide sequence encoding a SARS-COV-2 S protein or an antigenic fragment thereof is combined with a second nucleic acid comprising an optimized nucleotide sequence encoding a SARS-COV-2 N protein or an antigenic fragment thereof. In some embodiments, a first nucleic acid comprising an optimized nucleotide sequence encoding a SARS-COV-2 S protein or an antigenic fragment thereof is combined with a second nucleic acid comprising an optimized nucleotide sequence encoding a SARS-COV-2 E protein or an antigenic fragment thereof. In other embodiments, a first nucleic acid comprising an optimized nucleotide sequence encoding a SARS-CoV-2 S protein or an antigenic fragment thereof is combined with second, third and/or fourth nucleic acids, wherein said second nucleic acid comprises an optimized nucleotide sequence encoding a SARS-COV-2 M protein or an antigenic fragment thereof, wherein said third nucleic acid comprises an optimized nucleotide sequence encoding a SARS-COV-2 N protein or an antigenic fragment thereof, and wherein said fourth nucleic acid comprises an optimized nucleotide sequence encoding a SARS-COV-2 E protein or an antigenic fragment thereof.

mRNA Sequences

In some embodiments, an mRNA comprising an optimized nucleotide sequence encoding a SARS-COV-2 S protein or an antigenic fragment thereof also contains 5′ and 3′ UTR sequences. Exemplary 5′ and 3′ UTR sequences are shown below:

### Exemplary 5′ UTR Sequence

(SEQ ID NO: 144)
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG

### Exemplary 3′ UTR Sequence

(SEQ ID NO: 145)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UCAAGCU
OR
(SEQ ID NO: 146)
GGGUGGCAUCCCUGUGACCCCUCCCCAGUGCCUCUCCUGGCCCUGGAAG
UUGCCACUCCAGUGCCCACCAGCCUUGUCCUAAUAAAAUUAAGUUGCAU
CAAAGCU

Exemplary mRNA Constructs

In a particular embodiment, an mRNA comprising an optimized nucleotide sequence encoding a SARS-COV-2 S protein comprises the following structural elements:

TABLE 4
Structural elements of exemplary mRNA constructs

| | | |
|---|---|---|
| Structural | | Sequence |
| Element | Description | Coordinates |

mRNA construct 1

| | | |
|---|---|---|
| Cap Structure | 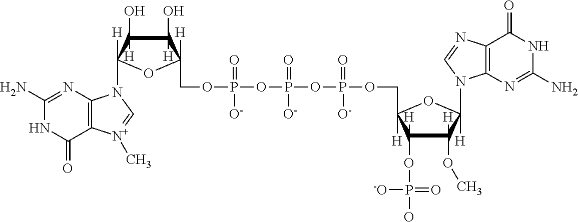 | 1 |
| 5′ UTR | GGAC . . . CACG | 1-140 |
| | | (SEQ ID NO: 144) |
| SARS-CoV- | AUG . . . UGA | 141-3962 |
| 2 S protein¹ | | (SEQ ID NO:148), |
| | | which corresponds |
| | | to the nucleotide |
| | | sequence of |
| | | SEQ ID NO: 44 |
| 3′ UTR | CGGG . . . AGCU | 3963-4067 |
| | | (SEQ ID NO: 145) |
| PolyA tail | (A)ₓ, x = 100-500³ | NA |

mRNA construct 2

| | | |
|---|---|---|
| Cap Structure | 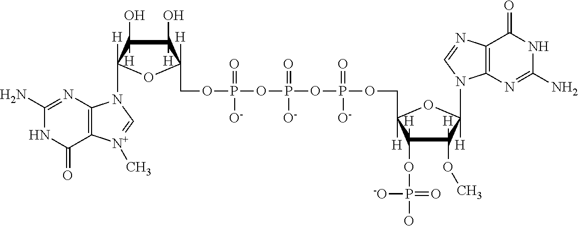 | 1 |
| 5′ UTR | GGAC . . .CACG | 1-140 |
| | | (SEQ ID NO: 144) |
| SARS-CoV- | AUG .... UGA | 141-3962 |
| 2 S protein² | | (SEQ ID NO:173), |
| | | which corresponds |
| | | to the nucleotide |
| | | sequence of |
| | | SEQ ID NO: 166 |
| 3′ UTR | CGGG . . . AGCU | 3963-4067 |
| | | (SEQ ID NO: 145) |
| Poly A tail | (A)ₓ, x = 100-500³ | NA |

NA = not applicable
UTR = untranslated region
¹Optimized nucleotide sequence encoding a SARS-CoV-2 S protein mutated to remove a furin cleavage site and to replace residues 986 and 987 with proline
²Optimized nucleotide sequence encoding a SARS-CoV-2 S protein mutated to remove a furin cleavage site and to replace residues 986 and 987 with proline and further containing the L18F, D80A, D215G, L242-, A243-, L244-, K417N, E484K, N501Y, D614G and A701V mutations
³expected range

In one particular embodiment, the mRNA in accordance with the present invention has the following nucleic acid sequence:

| (SEQ ID NO: 147) | |
|---|---|
| 1 | GGACAGAUCG CCUGGAGACG CCAUCCACGC UGUUUUGACC UCCAUAGAAG |
| 51 | ACACCGGGAC CGAUCCAGCC UCCGCGGCCG GGAACGGUGC AUUGGAACGC |
| 101 | GGAUUCCCCG UGCCAAGAGU GACUCACCGU CCUUGACACG AUGUUCGUCU |
| 151 | UCCUCGUGCU GCUCCCACUC GUUUCUUCCC AGUGUGUCAA CCUGACAACU |
| 201 | AGGACUCAGC UGCCACCAGC CUACACCAAC UCCUUCACCA GAGGCGUGUA |
| 251 | UUACCCAGAC AAGGUGUUUA GAAGCAGCGU GCUGCACUCU ACCCAGGACC |
| 301 | UCUUUCUGCC CUUUUUCAGC AACGUGACAU GGUUUCACGC AAUUCACGUG |
| 351 | UCCGGCACUA AUGGCACAAA GCGGUUCGAC AAUCCAGUCC UGCCUUUCAA |
| 401 | CGAUGGCGUC UACUUUGCAU CUACUGAGAA AUCCAAUAUC AUUAGGGGAU |
| 451 | GGAUCUUCGG CACAACCCUG GAUUCUAAGA CCCAGAGCCU GCUGAUCGUC |
| 501 | AACAACGCCA CAAACGUGGU CAUUAAGGUU UGCGAGUUUC AGUUCUGUAA |
| 551 | CGAUCCUUUU CUGGGCGUGU AUUAUCAUAA GAACAAUAAG AGCUGGAUGG |
| 601 | AGUCCGAGUU UAGAGUGUAU AGCUCUGCAA AUAAUUGUAC CUUUGAGUAC |
| 651 | GUGAGCCAGC CCUUUCUGAU GGACCUGGAG GGAAAACAAG GAAACUUCAA |
| 701 | AAACCUGCGG GAAUUCGUUU UCAAAAACAU CGACGGCUAU UUCAAGAUCU |
| 751 | AUAGCAAGCA UACCCCAAUC AACCUCGUGA GGGACCUCCC CCAGGGCUUU |
| 801 | AGCGCACUGG AGCCACUGGU UGACCUGCCU AUCGGCAUUA AUAUCACAAG |
| 851 | AUUUCAGACC CUGCUGGCAC UGCAUAGAAG CUAUCUGACC CCUGGAGACU |
| 901 | CCUCUAGUGG GUGGACUGCC GGCGCCGCUG CCUACUAUGU GGGCUAUCUG |
| 951 | CAGCCACGGA CAUUCCUGCU GAAAUACAAU GAGAACGGGA CAAUCACAGA |
| 1001 | UGCUGUUGAU UGCGCACUCG ACCCCCUGUC CGAGACAAAG UGCACUCUCA |
| 1051 | AGAGCUUUAC CGUCGAGAAG GGCAUCUAUC AGACCUCAAA CUUCAGGGUG |
| 1101 | CAGCCCACAG AAUCUAUCGU GCGCUUCCCU AAUAUCACUA ACCUGUGUCC |
| 1151 | UUUCGGUGAA GUGUUCAACG CCACCAGGUU UGCUAGCGUG UAUGCCUGGA |
| 1201 | ACAGGAAGAG GAUCUCUAAC UGCGUCGCCG ACUAUUCCGU GCUGUAUAAC |
| 1251 | AGCGCCUCCU UCUCCACAUU CAAAUGCUAU GGAGUGAGCC CGACAAAACU |
| 1301 | GAACGAUCUC UGCUUUACAA AUGUCUACGC CGACUCUUUU GUGAUCAGAG |
| 1351 | GGGACGAGGU CCGGCAGAUC GCACCAGGAC AGACAGGCAA GAUUGCUGAC |
| 1401 | UACAACUAUA AGCUGCCUGA CGACUUCACA GGAUGUGUGA UCGCAUGGAA |
| 1451 | CUCAAACAAU CUGGACUCCA AAGUCGGGGG CAACUAUAAU UACCUGUAUC |
| 1501 | GCCUGUUCCG GAAGUCCAAC CUGAAGCCCU UCGAGAGGGA CAUCAGUACA |
| 1551 | GAGAUCUAUC AGGCUGGCUC CACCCCUUGC AAUGGCGUCG AAGGCUUUAA |
| 1601 | UUGUUAUUUU CCCCUGCAGU CUUACGGGUU UCAGCCUACU AAUGGAGUUG |
| 1651 | GGUACCAGCC AUACAGAGUG GUCGUGCUCA GCUUCGAGCU CCUGCAUGCU |
| 1701 | CCAGCUACAG UUUGCGGGCC AAAGAAGUCC ACUAACCUGG UGAAGAAUAA |
| 1751 | GUGCGUCAAC UUCAACUUUA ACGGGCUCAC CGGCACCGGC GUGCUGACUG |
| 1801 | AGAGCAACAA GAAGUUUCUG CCAUUUCAAC AGUUUGGACG GGACAUUGCC |
| 1851 | GACACCACCG AUGCCGUUCG GGAUCCACAG ACCCUGGAAA UUCUGGACAU |
| 1901 | UACACCGUGC AGCUUCGGGG GCGUGAGCGU GAUCACACCC GGAACCAAUA |
| 1951 | CAAGCAACCA GGUUGCCGUC CUGUAUCAGG AUGUCAAUUG CACAGAAGUG |
| 2001 | CCAGUUGCUA UCCACGCAGA CCAGCUGACU CCCACAUGGC GGGUGUAUAG |
| 2051 | CACCGGAUCC AACGUGUUUC AGACCCGCGC CGGAUGUCUC AUUGGGGCCG |
| 2101 | AGCACGUGAA UAACAGCUAC GAGUGCGACA UCCCCAUUGG CGCCGGCAUU |
| 2151 | UGUGCGUCUU ACCAGACUCA GACCAACUCU CCUGGCUCCG CCUCUUCCGU |
| 2201 | UGCUAGUCAG UCUAUUAUUG CCUAUACCAU GAGCCUCGGA GCUGAGAAUA |
| 2251 | GCGUGGCCUA CUCCAAUAAU UCCAUCGCAA UCCCUACUAA CUUCACUAUU |
| 2301 | UCUGUGACCA CCGAGAUCCU GCCUGUGUCU AUGACUAAGA CUAGCGUUGA |
| 2351 | UUGUACCAUG UAUAUUUGUG GCGACUCUAC CGAAUGUUCU AACCUGCUGC |
| 2401 | UUCAGUACGG CUCAUUUUGC ACACAGCUGA ACAGAGCCCU GACUGGGAUC |
| 2451 | GCUGUGGAGC AGGACAAGAA CACACAGGAG GUGUUUGCAC AGGUGAAGCA |
| 2501 | GAUCUAUAAG ACCCCUCCUA UUAAGGAUUU CGGCGGAUUC AAUUUCUCAC |
| 2551 | AGAUUCUGCC AGACCCCAGU AAGCCUUCCA AGAGGAGCUU CAUCGAGGAU |
| 2601 | CUCCUGUUUA ACAAGGUGAC CCUGGCAGAC GCCGGCUUUA UUAAGCAAUA |
| 2651 | UGGGGAUUGC CUGGGCGACA UUGCUGCCAG AGACCUGAUU UGCGCCCAGA |
| 2701 | AAUUCAAUGG CCUCACAGUG CUGCCACCUC UGCUGACCGA CGAGAUGAUC |
| 2751 | GCUCAAUACA CUAGCGCACU GCUGGCCGGA ACCAUCACAU CAGGCUGGAC |
| 2801 | CUUCGGGGCC GGAGCAGCAC UGCAGAUUCC AUUCGCCAUG CAGAUGGCCU |
| 2851 | AUAGAUUCAA CGGCAUUGGC GUCACACAGA ACGUGCUGUA CGAAAACCAG |
| 2901 | AAGCUCAUCG CUAACCAGUU UAAUUCCGCA AUUGGAAAGA UCCAAGAUUC |
| 2951 | ACUCAGCUCA ACCGCCUCUG CACUCGGAAA GCUGCAGGAC GUGGUCAACC |
| 3001 | AGAAUGCUCA GGCCCUGAAC ACACUCGUCA AGCAGCUGUC CUCUAACUUU |
| 3051 | GGCGCUAUCA GCUCCGUUCU GAACGACAUU CUGAGCCGCC UGGAUCCCCC |
| 3101 | AGAGGCUGAA GUCCAGAUUG ACCGCCUGAU UACCGGCCGG CUGCAGUCUC |
| 3151 | UGCAAACAUA CGUGACCCAG CAGCUGAUCA GAGCAGCCGA GAUCCGGGCA |
| 3201 | UCCGCAAAUC UGGCAGCAAC UAAGAUGAGC GAAUGCGUGC UGGGCCAGUC |
| 3251 | CAAGCGGGUG GACUUUUGUG GCAAGGGCUA CCACCUGAUG AGCUUCCCCC |
| 3301 | AGAGCGCCCC ACAUGGCGUU GUUUUUCUGC ACGUGACCUA UGUCCCUGCU |
| 3351 | CAGGAAAAGA ACUUUACAAC UGCUCCUGCU AUCUGCCAUG ACGGCAAGGC |
| 3401 | CCACUUCCCA CGGGAGGGAG UGUUUGUGUC CAAUGGCACA CACUGGUUCG |
| 3451 | UGACCCAGAG GAACUUCUAU GAACCCCAGA UCAUCACCAC UGACAAUACC |
| 3501 | UUCGUGUCUG GAAAUUGCGA CGUCGUGAUC GGCAUCGUUA ACAACACCGU |
| 3551 | GUACGACCCU CUCCAGCCAG AGCUGGACUC CUUUAAGGAG GAACUGGAUA |
| 3601 | AGUAUUUUAA GAACCACACA AGCCCAGAUG UGGAUCUCGG GGACAUCUCC |
| 3651 | GGAAUUAACG CCUCCGUGGU GAAUAUCCAG AAGGAGAUUG ACCGCCUAAA |
| 3701 | UGAAGUUGCC AAGAACCUCA AUGAGUCUCU GAUUGAUCUG CAGGAACUGG |
| 3751 | GCAAGUAUGA GCAGUAUAUC AAAUGGCCCU GGUACAUUUG GCUGGGGUUU |
| 3801 | AUCGCCGGAC UGAUUGCCAU CGUCAUGGUG ACCAUCAUGC UGUGUUGCAU |
| 3851 | GACCUCCUGU UGUUCCUGUC UGAAGGGCUG CUGUAGUUGC GGCUCUUGCU |
| 3901 | GUAAAUUCGA CGAAGAUGAU AGCGAGCCCG UGCUGAAGGG CGUGAAGCUG |
| 3951 | CAUUAUACCU GACGGGUGGC AUCCCUGUGA CCCCUCCCCA GUGCCUCUCC |
| 4001 | UGGCCCUGGA AGUUGCCACU CCAGUGCCCA CCAGCCUUGU CCUAAUAAAA |
| 4051 | UUAAGUUGCA UCAAGCU |

### +Poly A Tail

Nucleic acids in bold denote start and stop codons

In another particular embodiment, the mRNA in accordance with the present invention has the following nucleic acid sequence:

| (SEQ ID NO: 172) | |
|---|---|
| 1 | GGACAGAUCG CCUGGAGACG CCAUCCACGC UGUUUUGACC UCCAUAGAAG |
| 51 | ACACCGGGAC CGAUCCAGCC UCCGCGGCCG GGAACGGUGC AUUGGAACGC |
| 101 | GGAUUCCCCG UGCCAAGAGU GACUCACCGU CCUUGACACG AUGUUCGUCU |
| 151 | UCCUCGUGCU GCUCCCACUC GUUUCUUCCC AGUGUGUCAA CUUCACAACU |
| 201 | AGGACUCAGC UGCCACCAGC CUACACCAAC UCCUUCACCA GAGGCGUGUA |
| 251 | UUACCCAGAC AAGGUGUUUA GAAGCAGCGU GCUGCACUCU ACCCAGGACC |
| 301 | UCUUUCUGCC CUUUUUCAGC AACGUGACAU GGUUUCACGC AAUUCACGUG |
| 351 | UCCGGCACUA AUGGCACAAA GCGGUUCGCC AAUCCAGUCC UGCCUUUCAA |
| 401 | CGAUGGCGUC UACUUUGCAU CUACUGAGAA AUCCAAUAUC AUUAGGGGAU |
| 451 | GGAUCUUCGG CACAACCCUG GAUUCUAAGA CCCAGAGCCU GCUGAUCGUC |
| 501 | AACAACGCCA CAAACGUGGU CAUUAAGGUU UGCGAGUUUC AGUUCUGUAA |
| 551 | CGAUCCUUUU CUGGGCGUGU AUUAUCAUAA GAACAAUAAG AGCUGGAUGG |
| 601 | AGUCCGAGUU UAGAGUGUAU AGCUCUGCAA AUAAUUGUAC CUUUGAGUAC |
| 651 | GUGAGCCAGC CCUUUCUGAU GGACCUGGAG GGAAAACAAG GAAACUUCAA |
| 701 | AAACCUGCGG GAAUUCGUUU UCAAAAACAU CGACGGCUAU UUCAAGAUCU |
| 751 | AUAGCAAGCA UACCCCAAUC AACCUCGUGA GGGGCCUCCC CCAGGGCUUU |
| 801 | AGCGCACUGG AGCCACUGGU UGACCUGCCU AUCGGCAUUA AUAUCACAAG |
| 851 | AUUUCAGACC CUGCAUAGAA GCUAUCUGAC CCCUGGAGAC UCCUCUAGUG |
| 901 | GGUGGACUGC CGGCGCCGCU GCCUACUAUG UGGGCUAUCU GCAGCCACGG |
| 951 | ACAUUCCUGC UGAAAUACAA UGAGAACGGG ACAAUCACAG AUGCUGUUGA |
| 1001 | UUGCGCACUC GACCCCCUGU CCGAGACAAA GUGCACUCUC AAGAGCUUUA |
| 1051 | CCGUCGAGAA GGGCAUCUAU CAGACCUCAA ACUUCAGGGU GCAGCCCACA |
| 1101 | GAAUCUAUCG UGCGCUUCCC UAAUAUCACU AACCUGUGUC CUUUCGGUGA |
| 1151 | AGUGUUCAAC GCCACCAGGU UUGCUAGCGU GUAUGCCUGG AACAGGAAGA |
| 1201 | GGAUCUCUAA CUGCGUCGCC GACUAUUCCG UGCUGUAUAA CAGCGCCUCC |
| 1251 | UUCUCCACAU UCAAAUGCUA UGGAGUGAGC CCGACAAAAC UGAACGAUCU |
| 1301 | CUGCUUUACA AAUGUCUACG CCGACUCUUU UGUGAUCAGA GGGGACGAGG |
| 1351 | UCCGGCAGAU CGCACCAGGA CAGACAGGCA ACAUUGCUGA CUACAACUAU |
| 1401 | AAGCUGCCUG ACGACUUCAC AGGAUGUGUG AUCGCAUGGA ACUCAAACAA |
| 1451 | UCUGGACUCC AAAGUCGGGG GCAACUAUAA UUACCUGUAU CGCCUGUUCC |
| 1501 | GGAAGUCCAA CCUGAAGCCC UUCGAGAGGG ACAUCAGUAC AGAGAUCUAU |
| 1551 | CAGGCUGGCU CCACCCCUUG CAAUGGCGUC AAGGGCUUUA AUUGUUAUUU |
| 1601 | UCCCCUGCAG UCUUACGGGU UUCAGCCUAC UUACGGAGUU GGGUACCAGC |
| 1651 | CAUACAGAGU GGUCGUGCUC AGCUUCGAGC UCCUGCAUGC UCCAGCUACA |
| 1701 | GUUUGCGGGC CAAAGAAGUC CACUAACCUG GUGAAGAAUA AGUGCGUCAA |
| 1751 | CUUCAACUUU AACGGGCUCA CCGGCACCGG CGUGCUGACU GAGAGCAACA |
| 1801 | AGAAGUUUCU GCCAUUUCAA CAGUUUGGAC GGGACAUUGC CGACACCACC |
| 1851 | GAUGCCGUUC GGGAUCCACA GACCCUGGAA AUUCUGGACA UUACACCGUG |
| 1901 | CAGCUUCGGG GGCGUGAGCG UGAUCACACC CGGAACCAAU ACAAGCAACC |
| 1951 | AGGUUGCCGU CCUGUAUCAG GGCGUCAAUU GCACAGAAGU GCCAGUUGCU |
| 2001 | AUCCACGCAG ACCAGCUGAC UCCCACAUGG CGGGUGUAUA GCACCGGAUC |
| 2051 | CAACGUGUUU CAGACCCGCG CCGGAUGUCU CAUUGGGGCC GAGCACGUGA |
| 2101 | AUAACAGCUA CGAGUGCGAC AUCCCCAUUG GCGCCGGCAU UUGUGCGUCU |
| 2151 | UACCAGACUC AGACCAACUC UCCUGGCUCC GCCUCUUCCG UUGCUAGUCA |
| 2201 | GUCUAUUAUU GCCUAUACCA UGAGCCUCGG AGUGGAGAAU AGCGUGGCCU |
| 2251 | ACUCCAAUAA UUCCAUCGCA AUCCCUACUA ACUUCACUAU UUCUGUGACC |
| 2301 | ACCGAGAUCC UGCCUGUGUC UAUGACUAAG ACUAGCGUUG AUUGUACCAU |
| 2351 | GUAUAUUUGU GGCGACUCUA CCGAAUGUUC UAACCUGCUG CUUCAGUACG |
| 2401 | GCUCAUUUUG CACACAGCUG AACAGAGCCC UGACUGGGAU CGCUGUGGAG |
| 2451 | CAGGACAAGA ACACACAGGA GGUGUUUGCA CAGGUGAAGC AGAUCUAUAA |
| 2501 | GACCCCUCCU AUUAAGGAUU UCGGCGGAUU CAAUUUCUCA CAGAUUCUGC |
| 2551 | CAGACCCCAG UAAGCCUUCC AAGAGGAGCU UCAUCGAGGA UCUCCUGUUU |
| 2601 | AACAAGGUGA CCCUGGCAGA CGCCGGCUUU AUUAAGCAAU AUGGGGAUUG |
| 2651 | CCUGGGCGAC AUUGCUGCCA GAGACCUGAU UUGCGCCCAG AAAUUCAAUG |
| 2701 | GCCUCACAGU GCUGCCACCU CUGCUGACCG ACGAGAUGAU CGCUCAAUAC |
| 2751 | ACUAGCGCAC UGCUGGCCGG AACCAUCACA UCAGGCUGGA CCUUCGGGGC |
| 2801 | CGGAGCAGCA CUGCAGAUUC CAUUCGCCAU GCAGAUGGCC UAUAGAUUCA |
| 2851 | ACGGCAUUGG CGUCACACAG AACGUGCUGU ACGAAAACCA GAAGCUCAUC |
| 2901 | GCUAACCAGU UUAAUUCCGC AAUUGGAAAG AUCCAAGAUU CACUCAGCUC |
| 2951 | AACCGCCUCU GCACUCGGAA AGCUGCAGGA CGUGGUCAAC CAGAAUGCUC |
| 3001 | AGGCCCUGAA CACACUCGUC AAGCAGCUGU CCUCUAACUU UGGCGCUAUC |
| 3051 | AGCUCCGUUC UGAACGACAU UCUGAGCCGC CUGGAUCCCC CAGAGGCUGA |
| 3101 | AGUCCAGAUU GACCGCCUGA UUACCGGCCG GCUGCAGUCU CUGCAAACAU |
| 3151 | ACGUGACCCA GCAGCUGAUC AGAGCAGCCG AGAUCCGGGC AUCCGCAAAU |
| 3201 | CUGGCAGCAA CUAAGAUGAG CGAAUGCGUG CUGGGCCAGU CCAAGCGGGU |
| 3251 | GGACUUUUGU GGCAAGGGCU ACCACCUGAU GAGCUUCCCC CAGAGCGCCC |
| 3301 | CACAUGGCGU UGUUUUUCUG CACGUGACCU AUGUCCCUGC UCAGGAAAAG |
| 3351 | AACUUUACAA CUGCUCCUGC UAUCUGCCAU GACGGCAAGG CCCACUUCCC |
| 3401 | ACGGGAGGGA GUGUUUGUGU CCAAUGGCAC ACACUGGUUC GUGACCCAGA |
| 3451 | GGAACUUCUA UGAACCCCAG AUCAUCACCA CUGACAAUAC CUUCGUGUCU |
| 3501 | GGAAAUUGCG ACGUCGUGAU CGGCAUCGUU AACAACACCG UGUACGACCC |
| 3551 | UCUCCAGCCA GAGCUGGACU CCUUUAAGGA GGAACUGGAU AAGUAUUUUA |
| 3601 | AGAACCACAC AAGCCCAGAU GUGGAUCUCG GGGACAUCUC CGGAAUUAAC |
| 3651 | GCCUCCGUGG UGAAUAUCCA GAAGGAGAUU GACCGCCUAA AUGAAGUUGC |
| 3701 | CAAGAACCUC AAUGAGUCUC UGAUUGAUCU GCAGGAACUG GGCAAGUAUG |
| 3751 | AGCAGUAUAU CAAAUGGCCC UGGUACAUUU GGCUGGGGUU UAUCGCCGGA |
| 3801 | CUGAUUGCCA UCGUCAUGGU GACCAUCAUG CUGUGUUGCA UGACCUCCUG |
| 3851 | UUGUUCCUGU CUGAAGGGCU GCUGUAGUUG CGGCUCUUGC UGUAAAUUCG |
| 3901 | ACGAAGAUGA UAGCGAGCCC GUGCUGAAGG GCGUGAAGCU GCAUUAUACC |
| 3951 | UGACGGGUGG CAUCCCUGUG ACCCCUCCCC AGUGCCUCUC CUGGCCCUGG |
| 4001 | AAGUUGCCAC UCCAGUGCCC ACCAGCCUUG UCCUAAUAAA AUUAAGUUGC |
| 4051 | AUCAAGCU |

### +Poly A Tail

Nucleic acids in bold denote start and stop codons
mRNA Synthesis

### In Vitro Transcription

mRNAs according to the present invention may be synthesized according to any of a variety of known methods. Various methods are described in published U.S. Application No. US 2018/0258423, and can be used to practice the present invention, all of which are incorporated herein by reference. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

### Nucleotides

In some embodiments, an mRNA comprises or consists of naturally-occurring nucleosides (or unmodified nucleosides; i.e., adenosine, guanosine, cytidine, and uridine). In some embodiments an mRNA comprises one or more modified nucleosides, such as nucleoside analogs (e.g. adenosine analog, guanosine analog, cytidine analog, or uridine analog). The presence of one or more nucleoside analogs may render an mRNA more stable and/or less immunogenic than a control mRNA with the same sequence but containing only naturally-occurring nucleosides. In a particular embodiment of the invention, mRNAs comprising an optimized nucleotide sequence encoding a SARS-COV-2 antigen are synthesized with naturally-occurring nucleosides. Without wishing to be bound by any particular theory, the inventors believe that the use of mRNAs prepared with naturally-occurring nucleosides is advantageous for providing an immunogenic composition of the invention.

In some embodiments, an mRNA comprises both unmodified and modified nucleosides. In some embodiments, the one or more modified nucleosides is a nucleoside analog. In some embodiments, the one or more modified nucleosides comprises at least one modification selected from a modified sugar, and a modified nucleobase. In some embodiments, the mRNA comprises one or more modified internucleoside linkages.

In some embodiments, the one or more modified nucleosides is a nucleoside analog, for example one of 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine (e.g., N-1-methyl-pseudouridine), 2-thiouridine, and 2-thiocytidine. See, e.g., U.S. Pat. No. 8,278,036 or WO 2011/012316 for a discussion of 5-methyl-cytidine, pseudouridine, and 2-thio-uridine and their incorporation into mRNA. In some embodiments, the mRNA may be RNA wherein 25% of U residues are 2-thio-uridine and 25% of C residues are 5-methylcytidine. Teachings for the use of such modified RNA are disclosed in US Patent Publication US 2012/0195936 and international publication WO 2011/012316, both of which are hereby incorporated by reference in their entirety.

### Post-Synthesis Processing

Typically, a 5′ cap and/or a 3′ tail may be added after mRNA synthesis. The presence of the cap is important in providing resistance to nucleases found in most eukaryotic cells. The presence of a “tail” serves to protect the mRNA from exonuclease degradation. Alternatively, the 5′ cap and/or a 3′ tail sequences are included in the DNA template sequences used in in vitro transcription reaction.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published U.S. Application No. US 2016/0032356 and published U.S. Application No. US 2018/0125989, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

### Post-Synthesis Purification

Various methods may be used to purify mRNA after synthesis. In some embodiments, the mRNA is purified using Tangential Flow Filtration. Suitable purification methods include those described in published U.S. Application No. US 2016/0040154, published U.S. Application No. US 2015/0376220, published U.S. Application No. US 2018/0251755, published U.S. Application No. US 2018/0251754, U.S. Provisional Application No. 62/757,612 filed on Nov. 8, 2018, and U.S. Provisional Application No. 62/891,781 filed on Aug. 26, 2019, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

### Lipid Nanoparticles

According to the present invention, an mRNA comprising an optimized nucleotide sequence of the invention may be delivered in a lipid nanoparticle. Typically, a lipid nanoparticle suitable for use with the present invention comprises one or more cationic lipids. In some embodiments, a lipid nanoparticle comprises one or more cationic lipids, one or more non-cationic lipids, one or more cholesterol-based lipids and one or more PEG-modified lipids. In some embodiments, a lipid nanoparticle comprises one or more cationic lipids, one or more non-cationic lipids, and one or more PEG-modified lipids. In some embodiments, a lipid nanoparticle comprises no more than four distinct lipid components.

A typical lipid nanoparticle for use with the invention is composed of four lipid components: a cationic lipid (e.g., a sterol-based cationic lipid), a non-cationic lipid (e.g., DOPE or DEPE), a cholesterol-based lipid (e.g., cholesterol) and a PEG-modified lipid (e.g., DMG-PEG2K). In some embodiments, a lipid nanoparticle comprises no more than three distinct lipid components. An exemplary lipid nanoparticle is composed of three lipid components: a cationic lipid (e.g., a sterol-based cationic lipid), a non-cationic lipid (e.g., DOPE or DEPE) and a PEG-modified lipid (e.g., DMG-PEG2K).

Formation of Lipid Nanoparticles Encapsulating mRNA

The lipid nanoparticles for use in the invention can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in published U.S. Application No. US 2011/0244026, published U.S. Application No. US 2016/0038432, published U.S. Application No. US 2018/0153822, published U.S. Application No. US 2018/0125989 and U.S. Provisional Application No. 62/877,597, filed Jul. 23, 2019 and can be used to practice the present invention, all of which are incorporated herein by reference. As used herein, Process A refers to a conventional method of encapsulating mRNA by mixing it with a mixture of lipids, without first pre-forming the lipids into lipid nanoparticles, as described in US 2016/0038432. As used herein, Process B refers to a process of encapsulating mRNA by mixing pre-formed lipid nanoparticles with mRNA, as described in US 2018/0153822.

Briefly, the process of preparing mRNA-loaded lipid nanoparticles includes a step of heating one or more of the solutions (i.e., applying heat from a heat source to the solution) to a temperature (or to maintain at a temperature) greater than ambient temperature, the one or more solutions being the solution comprising the pre-formed lipid nanoparticles, the solution comprising the mRNA and the mixed solution comprising the lipid nanoparticle encapsulated mRNA. In some embodiments, the process includes the step of heating one or both of the mRNA solution and the pre-formed lipid nanoparticle solution, prior to the mixing step. In some embodiments, the process includes heating one or more of the solution comprising the pre-formed lipid nanoparticles, the solution comprising the mRNA and the solution comprising the lipid nanoparticle encapsulated mRNA, during the mixing step. In some embodiments, the process includes the step of heating the lipid nanoparticle encapsulated mRNA, after the mixing step. In some embodiments, the temperature to which one or more of the solutions is heated (or at which one or more of the solutions is maintained) is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature greater than ambient temperature to which one or more of the solutions is heated is about 65° C.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps.

Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of or greater than about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

According to the present invention, a lipid solution contains a mixture of lipids suitable to form lipid nanoparticles for encapsulation of mRNA. In some embodiments, a suitable lipid solution is ethanol based. For example, a suitable lipid solution may contain a mixture of desired lipids dissolved in pure ethanol (i.e., 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. For example, a suitable lipid solution may contain a mixture of desired lipids at a total concentration of or greater than about 0.1 mg/ml, 0.5 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10 mg/ml, 15 mg/ml, 20 mg/ml, 30 mg/ml, 40 mg/ml, 50 mg/ml, or 100 mg/ml. In some embodiments, a suitable lipid solution may contain a mixture of desired lipids at a total concentration ranging from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml. In some embodiments, a suitable lipid solution may contain a mixture of desired lipids at a total concentration up to about 100 mg/ml, 90 mg/ml, 80 mg/ml, 70 mg/ml, 60 mg/ml, 50 mg/ml, 40 mg/ml, 30 mg/ml, 20 mg/ml, or 10 mg/ml.

Any desired lipids may be mixed at any ratios suitable for encapsulating mRNA. In some embodiments, a suitable lipid solution contains a mixture of desired lipids including cationic lipids, non-cationic lipids, cholesterol-based lipids, amphiphilic block copolymers (e.g. poloxamers) and/or PEG-modified lipids. In some embodiments, a suitable lipid solution contains a mixture of desired lipids including one or more cationic lipids, one or more non-cationic lipids, one or more cholesterol-based lipids, and/or one or more PEG-modified lipids.

In some embodiments, provided pharmaceutical compositions comprise a lipid nanoparticle wherein the mRNA are associated on both the surface of the lipid nanoparticle and encapsulated within the same lipid nanoparticle. For example, during preparation of the pharmaceutical compositions of the present invention, cationic lipid nanoparticles may associate with the mRNA through electrostatic interactions.

In some embodiments, the compounds, pharmaceutical compositions and methods of the invention comprise mRNA encapsulated in a lipid nanoparticle. In some embodiments, the mRNA may be encapsulated in the same lipid nanoparticle. In some embodiments, the mRNA may be encapsulated in different lipid nanoparticles. In some embodiments, the mRNA is encapsulated in one or more lipid nanoparticles, which differ in their lipid composition, molar ratio of lipid components, size, charge (zeta potential), targeting ligands and/or combinations thereof. In some embodiments, the one or more lipid nanoparticles may have a different composition of sterol-based cationic lipids, neutral lipids, PEG-modified lipids and/or combinations thereof. In some embodiments the one or more lipid nanoparticles may have a different molar ratio of cholesterol-based lipids, cationic lipids, neutral lipids, and PEG-modified lipids used to create the lipid nanoparticles.

The process of incorporation of a desired mRNA into a lipid nanoparticle is often referred to as “loading”. Exemplary methods are described in Lasic, et al. FEBS Lett., 312:255-258, 1992, which is incorporated herein by reference. The lipid nanoparticle-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely contained within the interior space of the lipid nanoparticle. The purpose of incorporating an mRNA into a lipid nanoparticle is often to protect the mRNA from an environment which may contain enzymes or chemicals that degrade mRNA and/or systems or receptors that cause the rapid excretion of the mRNA. Accordingly, in some embodiments, a suitable lipid nanoparticle is capable of enhancing the stability of the mRNA contained therein and/or facilitate the delivery of an mRNA to the target cell or tissue.

Suitable lipid nanoparticles in accordance with the present invention may be made in various sizes. In some embodiments, provided lipid nanoparticles may be made smaller than previously known lipid nanoparticles. In some embodiments, decreased size of lipid nanoparticles is associated with more efficient delivery of an mRNA. Selection of an appropriate lipid nanoparticle size may take into consideration the site of the target cell or tissue and to some extent the application for which the lipid nanoparticle is being made.

In some embodiments, an appropriate size of lipid nanoparticle is selected to facilitate systemic distribution of the mRNA. Alternatively or additionally, a lipid nanoparticle may be sized such that the dimensions of the lipid nanoparticle are of a sufficient diameter to limit or expressly avoid distribution into certain cells or tissues.

A variety of alternative methods known in the art are available for sizing of a population of lipid nanoparticles. One such sizing method is described in U.S. Pat. No. 4,737,323, incorporated herein by reference. Sonicating a lipid nanoparticles suspension either by bath or probe sonication produces a progressive size reduction down to small ULV less than about 0.05 microns in diameter. Homogenization is another method that relies on shearing energy to fragment large lipid nanoparticles into smaller ones. In a typical homogenization procedure, MLV are recirculated through a standard emulsion homogenizer until selected lipid nanoparticle sizes, typically between about 0.1 and 0.5 microns, are observed. The size of the lipid nanoparticles may be determined by quasi-electric light scattering (QELS) as described in Bloomfield, Ann. Rev. Biophys. Bioeng., 10:421-450 (1981), incorporated herein by reference. Average lipid nanoparticle diameter may be reduced by sonication of formed lipid nanoparticles. Intermittent sonication cycles may be alternated with QELS assessment to guide efficient lipid nanoparticle synthesis.

### Lipid Nanoparticle Formulations

In some embodiments, the majority of purified lipid nanoparticles in a pharmaceutical composition, i.e., greater than about 50%, 55%, 60%, 65%, 70%, 75%, 80%, 85%, 90%, 95%, 96%, 97%, 98%, or 99% of the lipid nanoparticles, have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all of the purified lipid nanoparticles have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In some embodiments, a lipid nanoparticle has an average size of less than 150 nm. In some embodiments, a lipid nanoparticle has an average size of less than 120 nm. In some embodiments, a lipid nanoparticle has an average size of less than 100 nm. In some embodiments, a lipid nanoparticle has an average size of less than 90 nm. In some embodiments, a lipid nanoparticle has an average size of less than 80 nm. In some embodiments, a lipid nanoparticle has an average size of less than 70 nm. In some embodiments, a lipid nanoparticle has an average size of less than 60 nm. In some embodiments, a lipid nanoparticle has an average size of less than 50 nm. In some embodiments, a lipid nanoparticle has an average size of less than 30 nm. In some embodiments, a lipid nanoparticle has an average size of less than 20 nm.

In some embodiments, greater than about 70%, 75%, 80%, 85%, 90%, 95%, 96%, 97%, 98%, 99% of the lipid nanoparticles in a pharmaceutical composition provided by the present invention have a size ranging from about 40-90 nm (e.g., about 45-85 nm, about 50-80 nm, about 55-75 nm, about 60-70 nm). In some embodiments, substantially all of the lipid nanoparticles have a size ranging from about 40-90 nm (e.g., about 45-85 nm, about 50-80 nm, about 55-75 nm, about 60-70 nm). Compositions with lipid nanoparticles having an average size of about 50-70 nm (e.g., 55-65 nm) are particular suitable for pulmonary delivery via nebulization.

In some embodiments, the dispersity, or measure of heterogeneity in size of molecules (PDI), of lipid nanoparticles in a pharmaceutical composition provided by the present invention is less than about 0.5. In some embodiments, a lipid nanoparticle has a PDI of less than about 0.5.

In some embodiments, a lipid nanoparticle has a PDI of less than about 0.4. In some embodiments, a lipid nanoparticle has a PDI of less than about 0.3. In some embodiments, a lipid nanoparticle has a PDI of less than about 0.28. In some embodiments, a lipid nanoparticle has a PDI of less than about 0.25. In some embodiments, a lipid nanoparticle has a PDI of less than about 0.23. In some embodiments, a lipid nanoparticle has a PDI of less than about 0.20. In some embodiments, a lipid nanoparticle has a PDI of less than about 0.18. In some embodiments, a lipid nanoparticle has a PDI of less than about 0.16. In some embodiments, a lipid nanoparticle has a PDI of less than about 0.14. In some embodiments, a lipid nanoparticle has a PDI of less than about 0.12. In some embodiments, a lipid nanoparticle has a PDI of less than about 0.10. In some embodiments, a lipid nanoparticle has a PDI of less than about 0.08.

In some embodiments, greater than about 75%, 80%, 85%, 90%, 95%, 96%, 97%, 98%, or 99% of the purified lipid nanoparticles in a pharmaceutical composition provided by the present invention encapsulate an mRNA within each individual particle. In some embodiments, substantially all of the purified lipid nanoparticles in a pharmaceutical composition encapsulate an mRNA within each individual particle. In some embodiments, a lipid nanoparticle has an encapsulation efficiency of between 50% and 99%. In some embodiments, a lipid nanoparticle has an encapsulation efficiency of greater than about 60%. In some embodiments, a lipid nanoparticle has an encapsulation efficiency of greater than about 65%. In some embodiments, a lipid nanoparticle has an encapsulation efficiency of greater than about 70%. In some embodiments, a lipid nanoparticle has an encapsulation efficiency of greater than about 75%. In some embodiments, a lipid nanoparticle has an encapsulation efficiency of greater than about 80%. In some embodiments, a lipid nanoparticle has an encapsulation efficiency of greater than about 85%. In some embodiments, a lipid nanoparticle has an encapsulation efficiency of greater than about 90%. In some embodiments, a lipid nanoparticle has an encapsulation efficiency of greater than about 92%. In some embodiments, a lipid nanoparticle has an encapsulation efficiency of greater than about 95%. In some embodiments, a lipid nanoparticle has an encapsulation efficiency of greater than about 98%. In some embodiments, a lipid nanoparticle has an encapsulation efficiency of greater than about 99%. Typically, lipid nanoparticles for use with the invention have an encapsulation efficiency of at least 90%-95%.

In some embodiments, a lipid nanoparticle has a N/P ratio of between 1 and 10. In some embodiments, a lipid nanoparticle has a N/P ratio above 1. In some embodiments, a lipid nanoparticle has a N/P ratio of about 1. In some embodiments, a lipid nanoparticle has a N/P ratio of about 2. In some embodiments, a lipid nanoparticle has a N/P ratio of about 3. In some embodiments, a lipid nanoparticle has a N/P ratio of about 4. In some embodiments, a lipid nanoparticle has a N/P ratio of about 5. In some embodiments, a lipid nanoparticle has a N/P ratio of about 6. In some embodiments, a lipid nanoparticle has a N/P ratio of about 7. In some embodiments, a lipid nanoparticle has a N/P ratio of about 8. A typical lipid nanoparticle for use with the invention has an N/P ratio of about 4.

In some embodiments, a pharmaceutical composition according to the present invention contains at least about 0.5 mg, 1 mg, 5 mg, 10 mg, 100 mg, 500 mg, or 1000 mg of encapsulated mRNA. In some embodiments, a pharmaceutical composition contains about 0.1 mg to 1000 mg of encapsulated mRNA. In some embodiments, a pharmaceutical composition contains at least about 0.5 mg of encapsulated mRNA. In some embodiments, a pharmaceutical composition contains at least about 0.8 mg of encapsulated mRNA. In some embodiments, a pharmaceutical composition contains at least about 1 mg of encapsulated mRNA. In some embodiments, a pharmaceutical composition contains at least about 5 mg of encapsulated mRNA. In some embodiments, a pharmaceutical composition contains at least about 8 mg of encapsulated mRNA.

In some embodiments, a pharmaceutical composition contains at least about 10 mg of encapsulated mRNA. In some embodiments, a pharmaceutical composition contains at least about 50 mg of encapsulated mRNA. In some embodiments, a pharmaceutical composition contains at least about 100 mg of encapsulated mRNA. In some embodiments, a pharmaceutical composition contains at least about 500 mg of encapsulated mRNA. In some embodiments, a pharmaceutical composition contains at least about 1000 mg of encapsulated mRNA.

### Cationic Lipids

Suitable cationic lipids for use in the pharmaceutical compositions and methods of the invention include the cationic lipids as described in International Patent Publication WO 2010/144740, which is incorporated herein by reference. In some embodiments, the pharmaceutical compositions and methods of the present invention include a cationic lipid, (6Z,9Z,28Z,31Z)-heptatriaconta-6,9,28,31-tetraen-19-yl 4-(dimethylamino) butanoate, having a compound structure of:

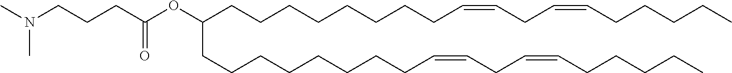 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the pharmaceutical compositions and methods of the present invention include ionizable cationic lipids as described in International Patent Publication WO 2013/149140, which is incorporated herein by reference. In some embodiments, the pharmaceutical compositions and methods of the present invention include a cationic lipid of one of the following formulas:

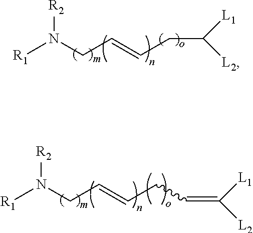

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In some embodiments, the pharmaceutical compositions and methods of the present invention include the cationic lipid (15Z, 18Z)—N,N-dimethyl-6-(9Z,12Z)-octadeca-9,12-dien-1-yl) tetracosa-15,18-dien-1-amine (“HGT5000”), having a compound structure of:

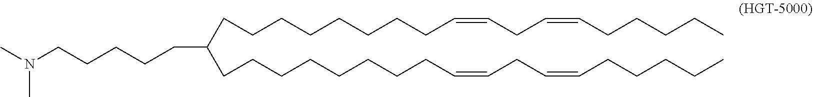 [SMILES: CCCCC/C=C\C/C=C\CCCCCCCCC(CCCCCCCC/C=C\C/C=C\CCCCC)CCCCCN(C)C]

and pharmaceutically acceptable salts thereof. In some embodiments, the pharmaceutical compositions and methods of the present invention include the cationic lipid (15Z, 18Z)—N,N-dimethyl-6-((9Z,12Z)-octadeca-9,12-dien-1-yl) tetracosa-4,15,18-trien-1-amine (“HGT5001”), having a compound structure of:

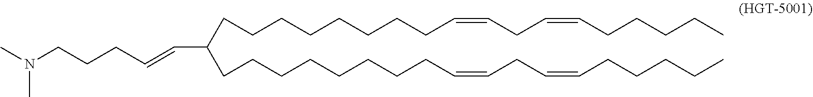 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In some embodiments, the pharmaceutical compositions and methods of the present invention include the cationic lipid and (15Z,18Z)—N,N-dimethyl-6-((9Z,12Z)-octadeca-9,12-dien-1-yl) tetracosa-5,15,18-trien-1-amine (“HGT5002”), having a compound structure of:

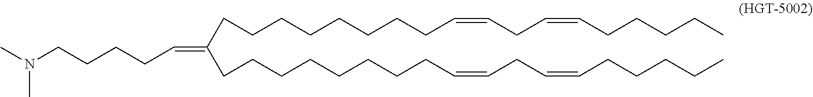 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the pharmaceutical compositions and methods of the invention include cationic lipids described as aminoalcohol lipidoids in International Patent Publication WO 2010/053572, which is incorporated herein by reference. In some embodiments, the pharmaceutical compositions and methods of the present invention include a cationic lipid having a compound structure of:

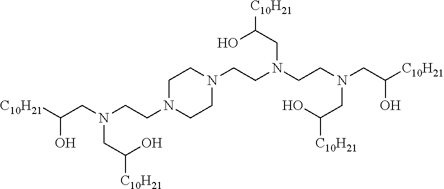

and pharmaceutically acceptable salts thereof.

Other suitable cationic lipids for use in the pharmaceutical compositions and methods of the invention include the cationic lipids as described in International Patent Publication WO 2016/118725, which is incorporated herein by reference. In some embodiments, the pharmaceutical compositions and methods of the present invention include a cationic lipid having a compound structure of:

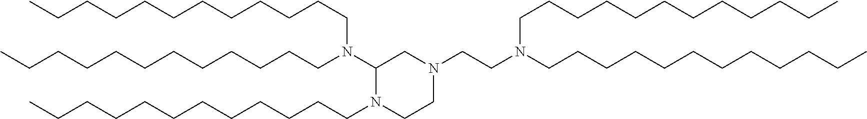 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the pharmaceutical compositions and methods of the invention include the cationic lipids as described in International Patent Publication WO 2016/118724, which is incorporated herein by reference. In some embodiments, the pharmaceutical compositions and methods of the present invention include a cationic lipid having a compound structure of:

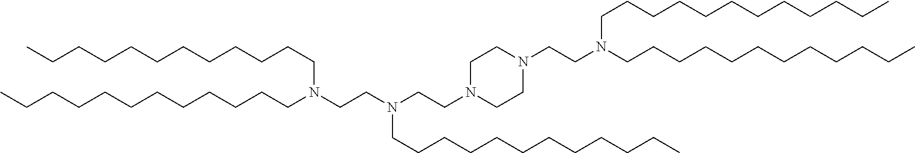 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the pharmaceutical compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the pharmaceutical compositions and methods of the invention include the cationic lipids as described in International Patent Publications WO 2013/063468 and WO 2016/205691, each of which are incorporated herein by reference. In some embodiments, the pharmaceutical compositions and methods of the present invention include a cationic lipid of the following formula:

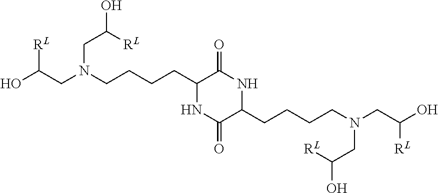

or pharmaceutically acceptable salts thereof, wherein each instance of R^{L} is independently optionally substituted C₆-C₄₀ alkenyl. In some embodiments, the pharmaceutical compositions and methods of the present invention include a cationic lipid having a compound structure of:

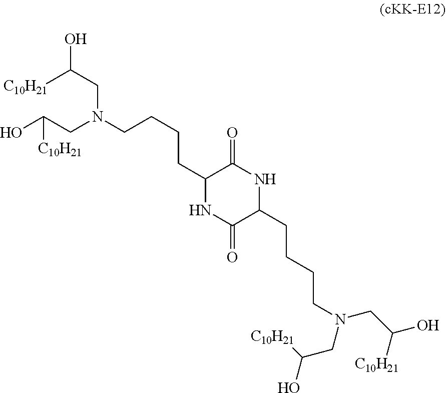

and pharmaceutically acceptable salts thereof. In some embodiments, the pharmaceutical compositions and methods of the present invention include a cationic lipid having a compound structure of:

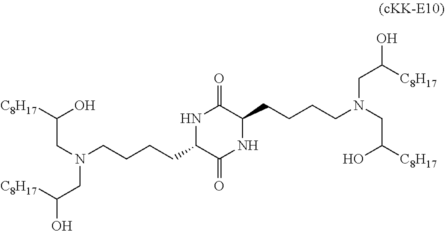

and pharmaceutically acceptable salts thereof.
In some embodiments, the pharmaceutical compositions and methods of the present invention include a cationic lipid having a compound structure of:

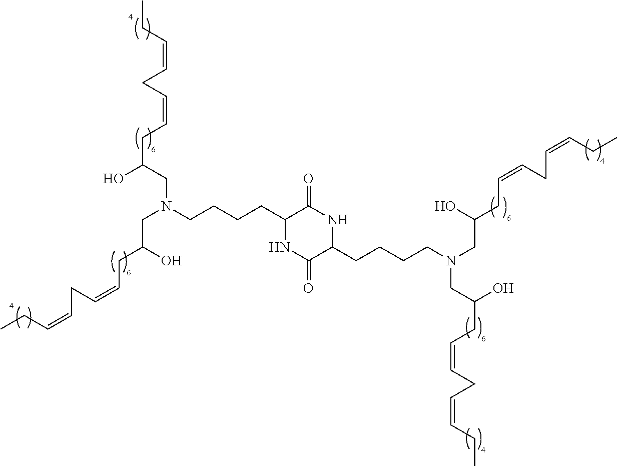

and pharmaceutically acceptable salts thereof. In some embodiments, the pharmaceutical compositions and methods of the present invention include a cationic lipid having a compound structure of:

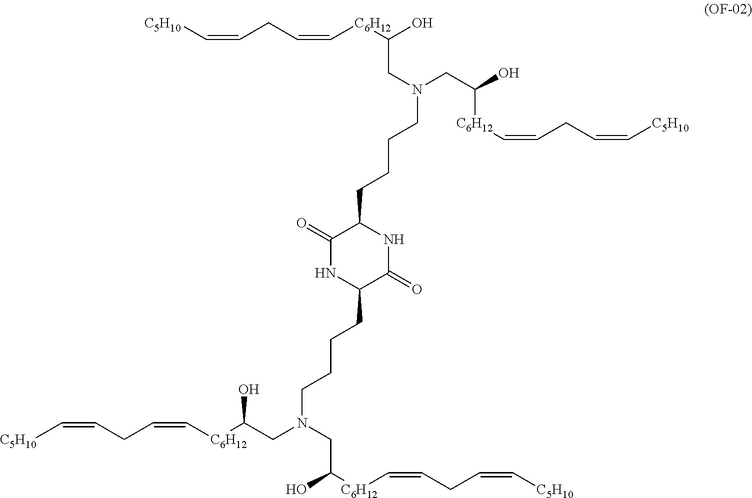

and pharmaceutically acceptable salts thereof. In some embodiments, the pharmaceutical compositions and methods of the present invention include a cationic lipid having a compound structure of:

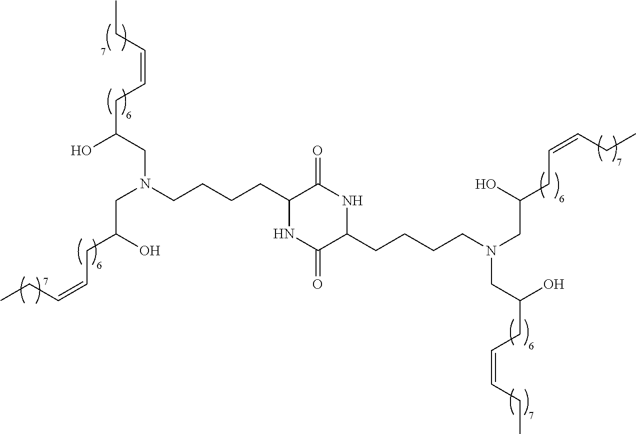

and pharmaceutically acceptable salts thereof. In some embodiments, the pharmaceutical compositions and methods of the present invention include a cationic lipid having a compound structure of:

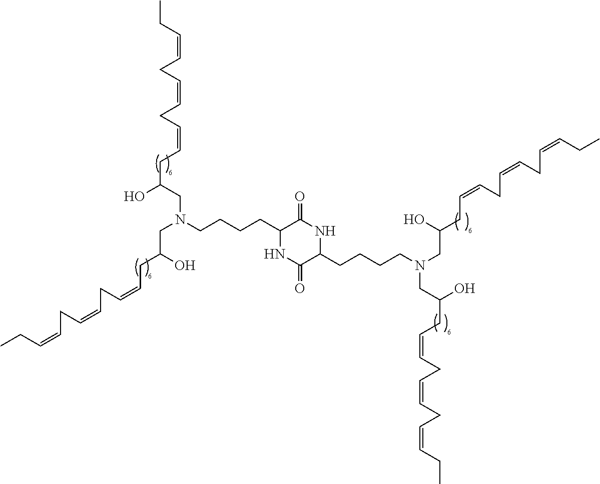

and pharmaceutically acceptable salts thereof.

Other suitable cationic lipids for use in the pharmaceutical compositions and methods of the invention include the cationic lipids as described in International Patent Publication WO 2015/184256, which is incorporated herein by reference. In some embodiments, the pharmaceutical compositions and methods of the present invention include a cationic lipid of the following formula:

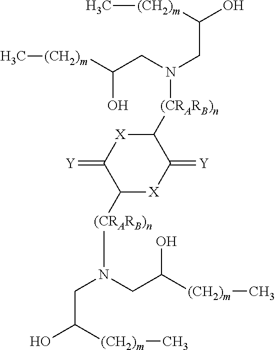

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each R_{B} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In some embodiments, the pharmaceutical compositions and methods of the present invention include a cationic lipid, “Target 23”, having a compound structure of:

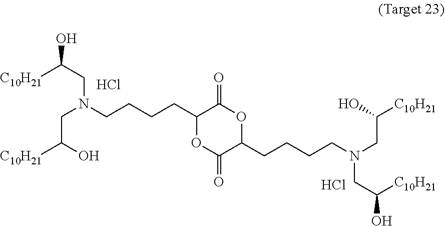

and pharmaceutically acceptable salts thereof.

Other suitable cationic lipids for use in the pharmaceutical compositions and methods of the invention include the cationic lipids as described in International Patent Publication WO 2016/004202, which is incorporated herein by reference. In some embodiments, the pharmaceutical compositions and methods of the present invention include a cationic lipid having the compound structure:

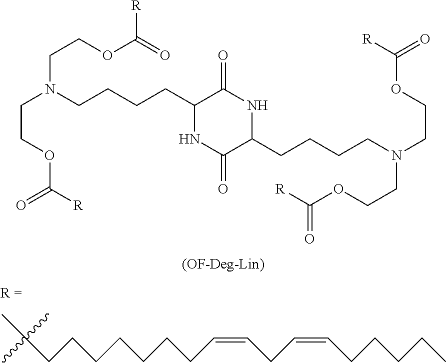

or a pharmaceutically acceptable salt thereof. In some embodiments, the pharmaceutical compositions and methods of the present invention include a cationic lipid having the compound structure:

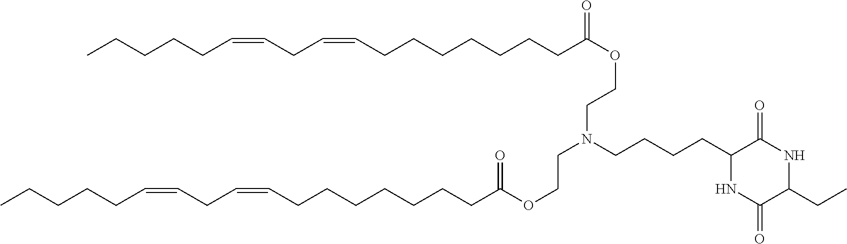 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the pharmaceutical compositions and methods of the present invention include a cationic lipid having the compound structure:

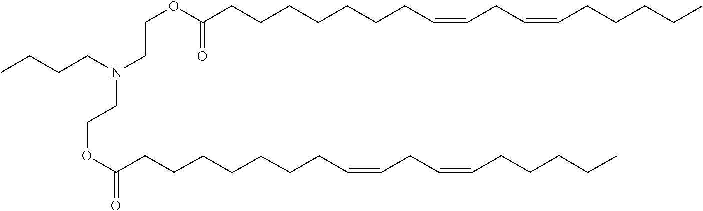 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the pharmaceutical compositions and methods of the present invention include cationic lipids as described in U.S. Provisional Patent Application Ser. No. 62/758,179, filed on Nov. 9, 2018, and Provisional Patent Application Ser. No. 62/871,510, filed on Jul. 8, 2019, which are incorporated herein by reference. In some embodiments, the pharmaceutical compositions and methods of the present invention include a cationic lipid of the following formula:

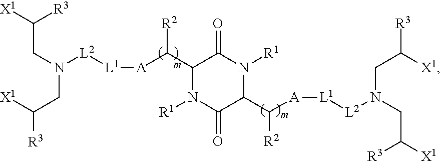

or a pharmaceutically acceptable salt thereof, wherein each R¹ and R² is independently H or C₁-C₆ aliphatic; each m is independently an integer having a value of 1 to 4; each A is independently a covalent bond or arylene; each L′ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the pharmaceutical compositions and methods of the present invention include a cationic lipid of the following formula:

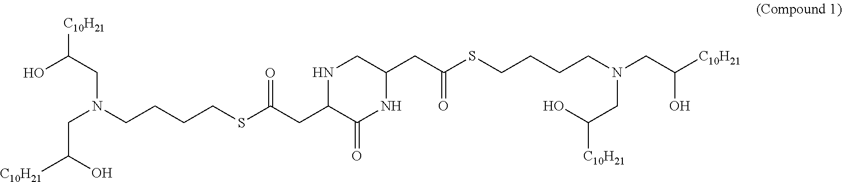

or a pharmaceutically acceptable salt thereof. In some embodiments, the pharmaceutical compositions and methods of the present invention include a cationic lipid of the following formula:

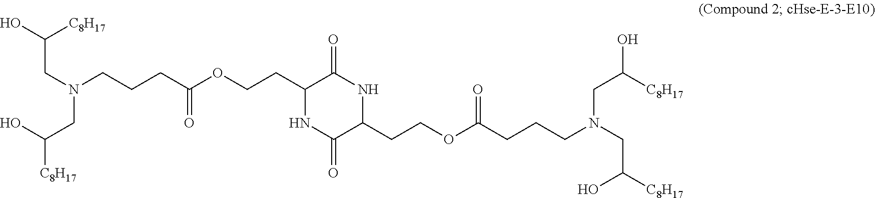

or a pharmaceutically acceptable salt thereof. In some embodiments, the pharmaceutical compositions and methods of the present invention include a cationic lipid of the following formula:

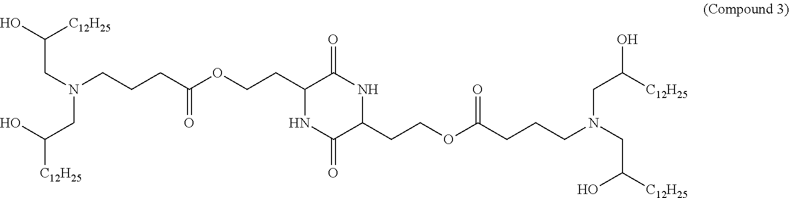

or a pharmaceutically acceptable salt thereof.

Other suitable cationic lipids for use in the pharmaceutical compositions and methods of the present invention include the cationic lipids as described in J. McClellan, M. C. King, Cell 2010, 141, 210-217 and in Whitehead et al., Nature Communications (2014) 5:4277, which is incorporated herein by reference. In some embodiments, the cationic lipids of the pharmaceutical compositions and methods of the present invention include a cationic lipid having a compound structure of:

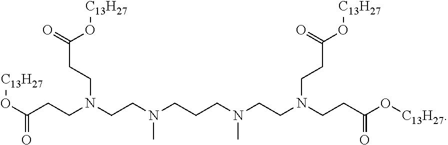

and pharmaceutically acceptable salts thereof.

Other suitable cationic lipids for use in the pharmaceutical compositions and methods of the invention include the cationic lipids as described in International Patent Publication WO 2015/199952, which is incorporated herein by reference. In some embodiments, the pharmaceutical compositions and methods of the present invention include a cationic lipid having the compound structure:

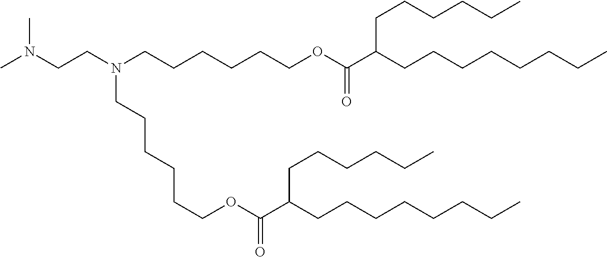 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the pharmaceutical compositions and methods of the present invention include a cationic lipid having the compound structure:

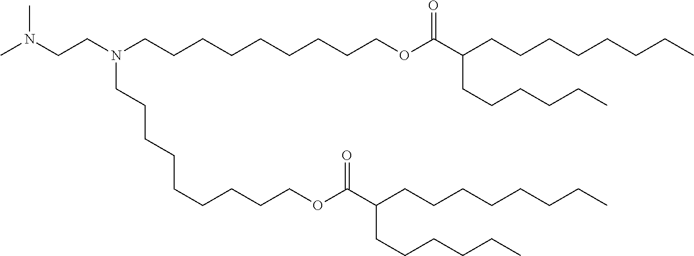 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the pharmaceutical compositions and methods of the present invention include a cationic lipid having the compound structure:

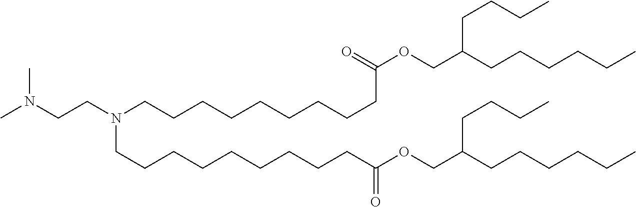 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the pharmaceutical compositions and methods of the present invention include a cationic lipid having the compound

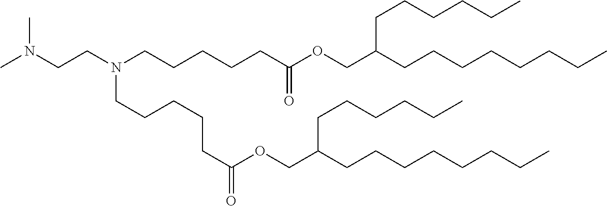 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the pharmaceutical compositions and methods of the present invention include a cationic lipid having the compound structure:

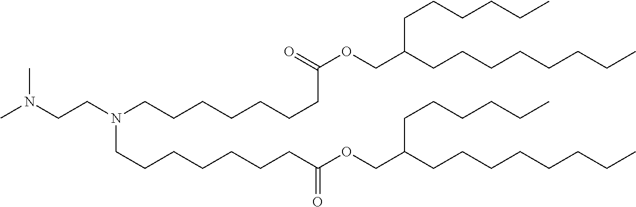 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the pharmaceutical compositions and methods of the present invention include a cationic lipid having the compound structure:

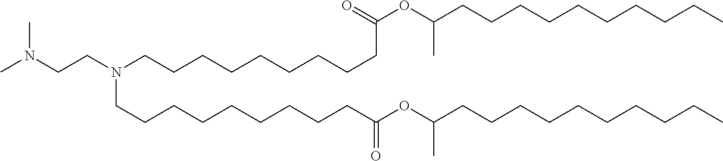 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the pharmaceutical compositions and methods of the present invention include a cationic lipid having the compound structure:

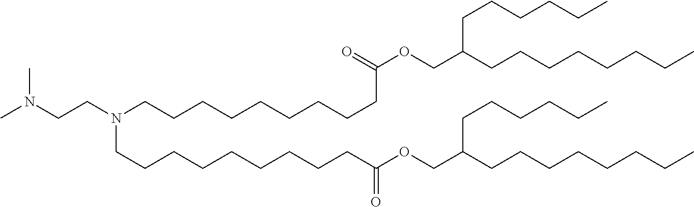 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the pharmaceutical compositions and methods of the present invention include a cationic lipid having the compound structure:

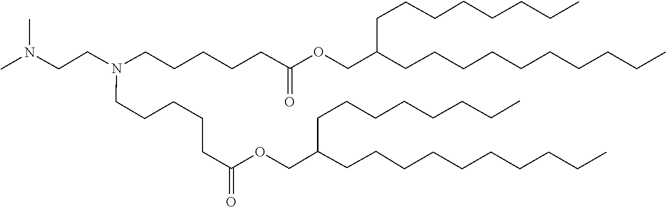 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the pharmaceutical compositions and methods of the present invention include a cationic lipid having the compound structure:

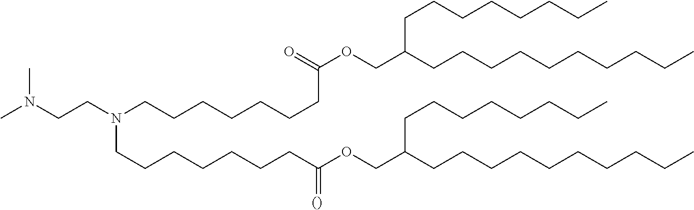

and pharmaceutically acceptable salts thereof. In some embodiments, the pharmaceutical compositions and methods of the present invention include a cationic lipid having the compound structure:

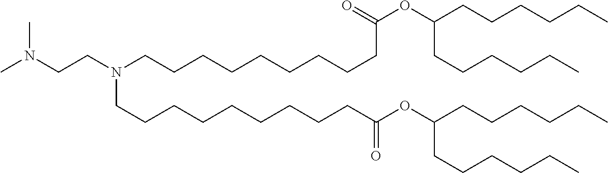 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the pharmaceutical compositions and methods of the present invention include a cationic lipid having the compound

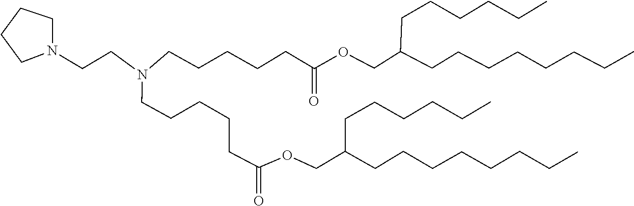 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the pharmaceutical compositions and methods of the present invention include a cationic lipid having the compound structure:

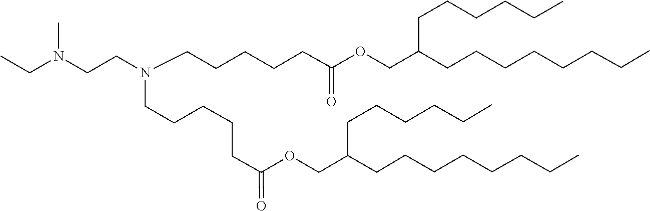 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the pharmaceutical compositions and methods of the present invention include a cationic lipid having the compound structure:

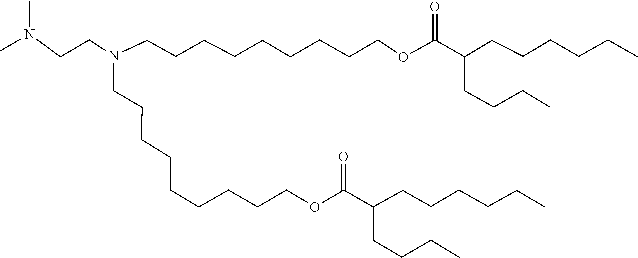

and pharmaceutically acceptable salts thereof.

Other suitable cationic lipids for use in the pharmaceutical compositions and methods of the invention include the cationic lipids as described in International Patent Publication WO 2017/004143, which is incorporated herein by reference. In some embodiments, the pharmaceutical compositions and methods of the present invention include a cationic lipid having the compound structure:

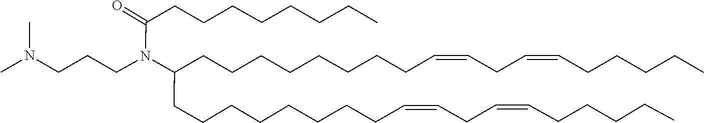 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the pharmaceutical compositions and methods of the present invention include a cationic lipid having the compound structure:

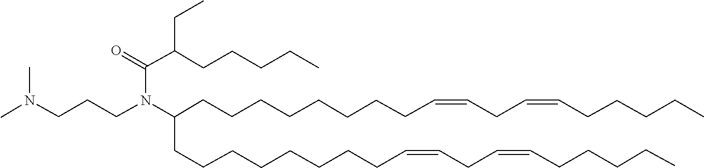 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the pharmaceutical compositions and methods of the present invention include a cationic lipid having the compound structure:

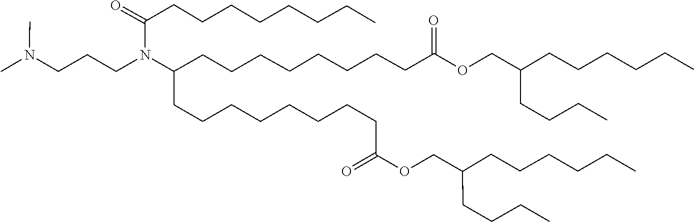 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the pharmaceutical compositions and methods of the present invention include a cationic lipid having the compound structure:

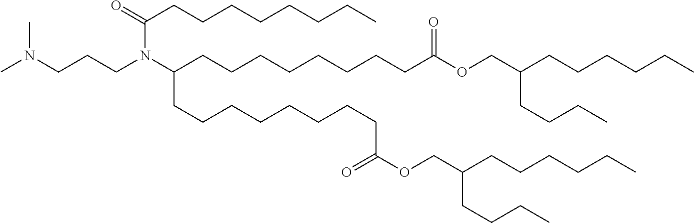 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the pharmaceutical compositions and methods of the present invention include a cationic lipid having the compound

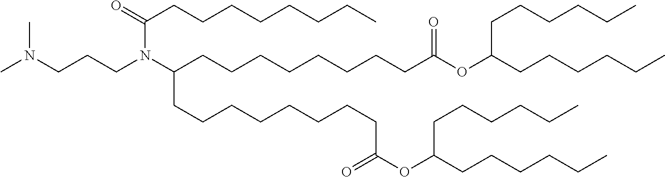 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the pharmaceutical compositions and methods of the present invention include a cationic lipid having the compound structure:

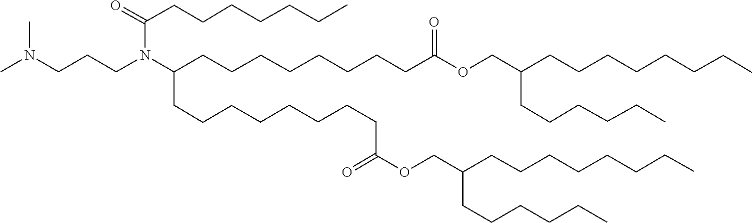 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the pharmaceutical compositions and methods of the present invention include a cationic lipid having the compound structure:

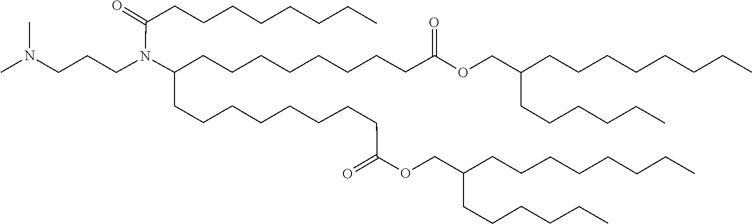 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the pharmaceutical compositions and methods of the present invention include a cationic lipid having the compound structure:

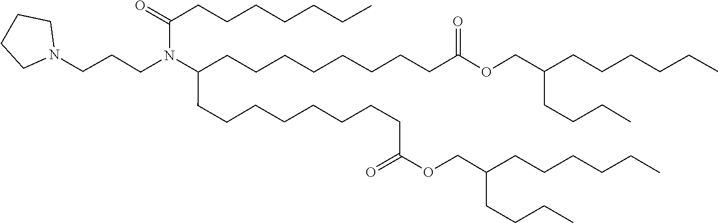 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the pharmaceutical compositions and methods of the present invention include a cationic lipid having the compound

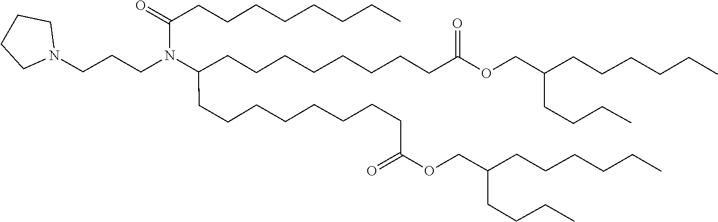 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the pharmaceutical compositions and methods of the present invention include a cationic lipid having the compound structure:

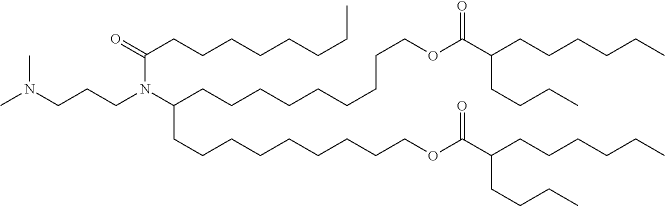 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the pharmaceutical compositions and methods of the present invention include a cationic lipid having the compound structure:

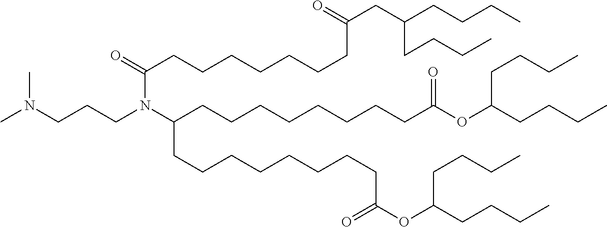 [SMILES: CCCCC(CCCC)CC(=O)CCCCCCCC(=O)N(CCCN(C)C)C(CCCCCCCCC(=O)OC(CCCC)CCCC)CCCCCCCCC(=O)OC(CCCC)CCCC]

and pharmaceutically acceptable salts thereof. In some embodiments, the pharmaceutical compositions and methods of the present invention include a cationic lipid having the compound

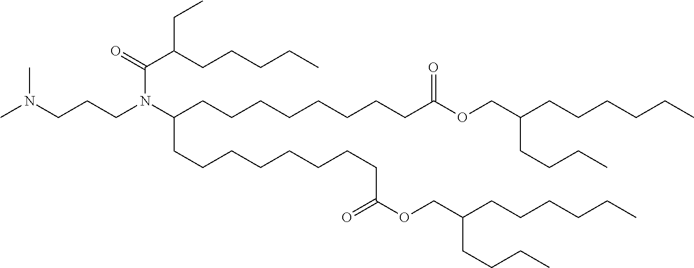 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the pharmaceutical compositions and methods of the present invention include a cationic lipid having the compound structure:

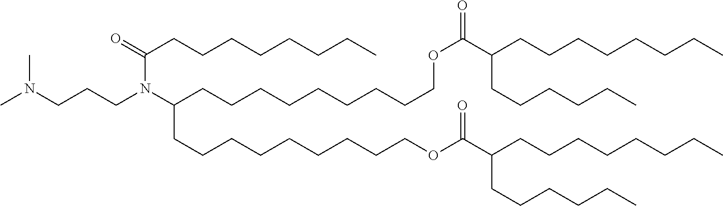 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the pharmaceutical compositions and methods of the present invention include a cationic lipid having the compound structure:

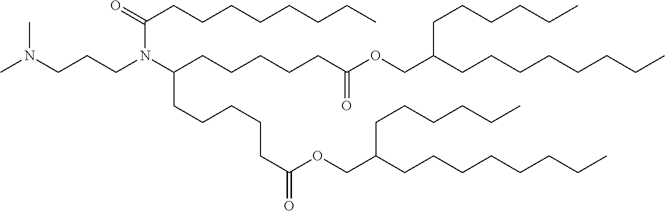 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the pharmaceutical compositions and methods of the present invention include a cationic lipid having the compound

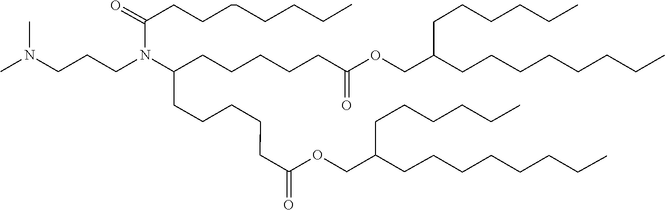 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the pharmaceutical compositions and methods of the present invention include a cationic lipid having the compound structure:

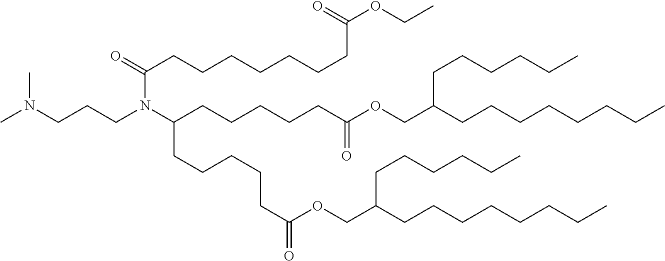 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the pharmaceutical compositions and methods of the present invention include a cationic lipid having the compound structure:

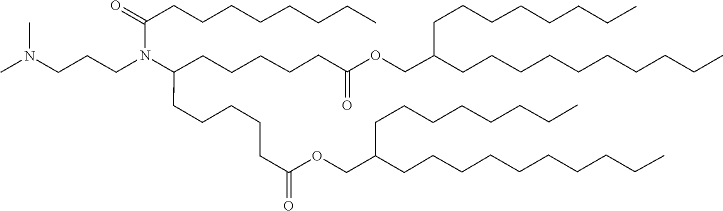 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the pharmaceutical compositions and methods of the invention include the cationic lipids as described in International Patent Publication WO 2017/075531, which is incorporated herein by reference. In some embodiments, the pharmaceutical compositions and methods of the present invention include a cationic lipid of the following formula:

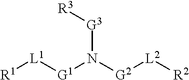

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the pharmaceutical compositions and methods of the invention include the cationic lipids as described in International Patent Publication WO 2017/117528, which is incorporated herein by reference. In some embodiments, the pharmaceutical compositions and methods of the present invention include a cationic lipid having the compound structure:

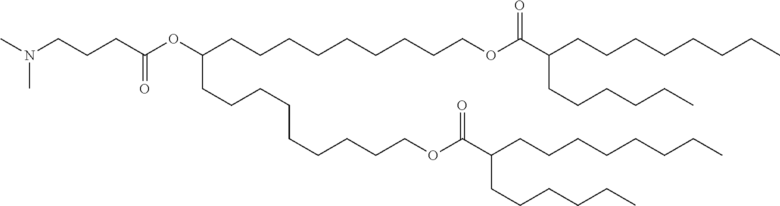 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the pharmaceutical compositions and methods of the present invention include a cationic lipid having the compound structure:

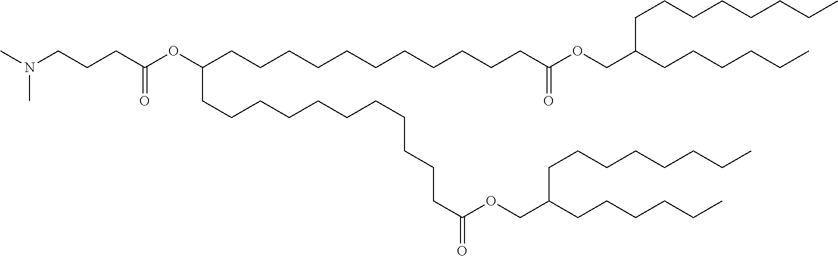 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the pharmaceutical compositions and methods of the present invention include a cationic lipid having the compound structure:

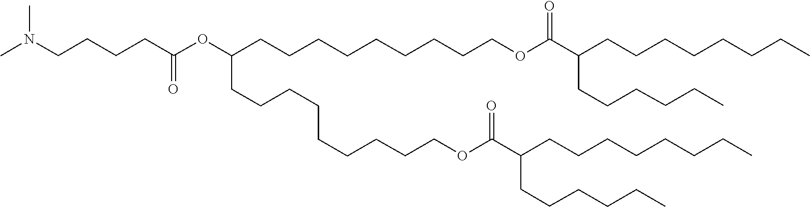 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the pharmaceutical compositions and methods of the invention include the cationic lipids as described in International Patent Publication WO 2017/049245, which is incorporated herein by reference. In some embodiments, the cationic lipids of the pharmaceutical compositions and methods of the present invention include a compound of one of the following formulas:

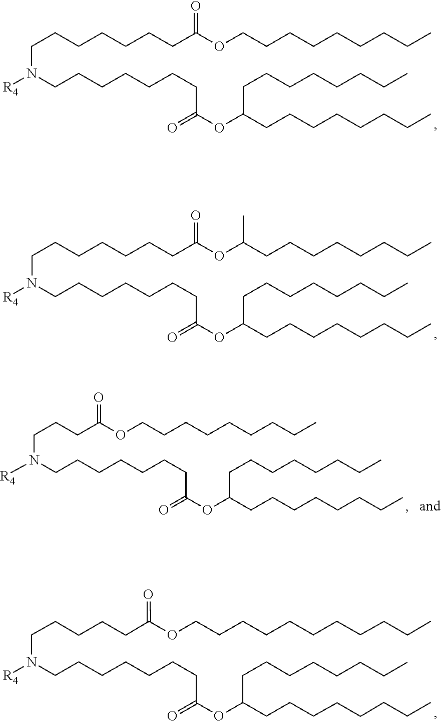

and pharmaceutically acceptable salts thereof. For any one of these four formulas, R⁴ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R) C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In some embodiments, the pharmaceutical compositions and methods of the present invention include a cationic lipid having a compound structure of:

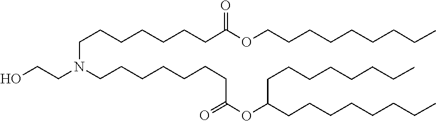 [SMILES: CCCCCCCCCOC(=O)CCCCCCCN(CCO)CCCCCCCC(=O)OC(CCCCCCCC)CCCCCCCC]

and pharmaceutically acceptable salts thereof. In some embodiments, the pharmaceutical compositions and methods of the present invention include a cationic lipid having a compound structure of:

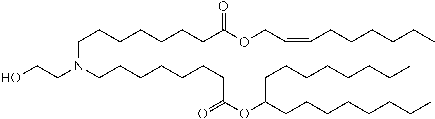 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In some embodiments, the pharmaceutical compositions and methods of the present invention include a cationic lipid having a compound structure of:

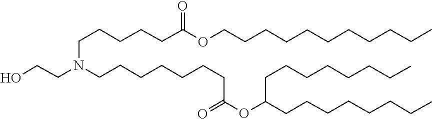 [SMILES: CCCCCCCCCCCOC(=O)CCCCCN(CCO)CCCCCCCC(=O)OC(CCCCCCCC)CCCCCCCC]

pharmaceutically acceptable salts thereof. In some embodiments, the pharmaceutical compositions and methods of the present invention include a cationic lipid having a compound structure of:

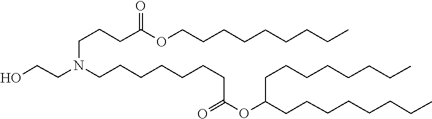 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the pharmaceutical compositions and methods of the invention include the cationic lipids as described in International Patent Publication WO 2017/173054 and WO 2015/095340, each of which is incorporated herein by reference. In some embodiments, the pharmaceutical compositions and methods of the present invention include a cationic lipid having a compound structure of:

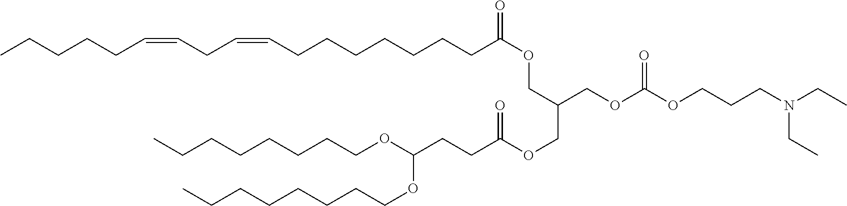 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In some embodiments, the pharmaceutical compositions and methods of the present invention include a cationic lipid having a compound structure of:

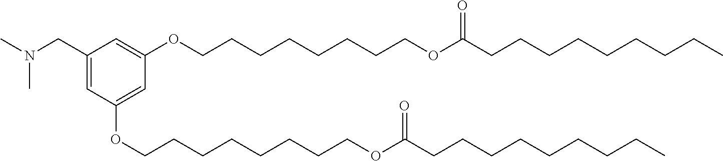 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In some embodiments, the pharmaceutical compositions and methods of the present invention include a cationic lipid having a compound structure of:

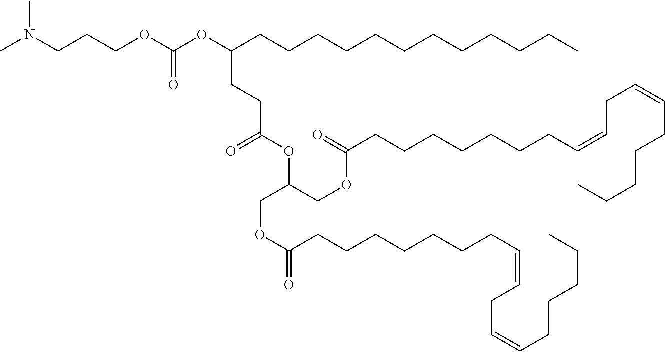 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In some embodiments, the pharmaceutical compositions and methods of the present invention include a cationic lipid having a compound structure of:

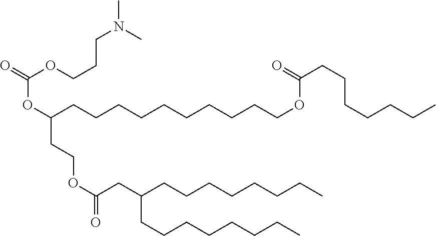 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the pharmaceutical compositions and methods of the present invention include cationic lipids as described in U.S. Provisional Patent Application Ser. No. 62/865,555, filed on Jun. 24, 2019, which is incorporated herein by reference. In some embodiments, the pharmaceutical compositions and methods of the present invention include a cationic lipid having a compound structure of:

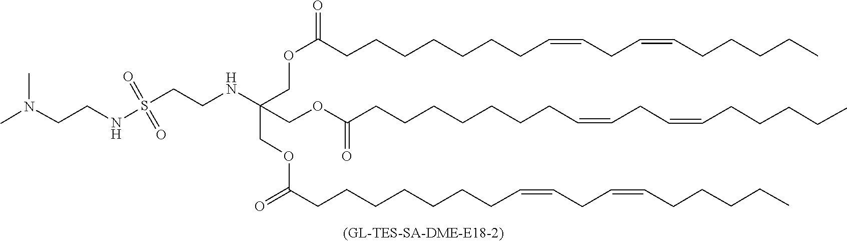 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

and pharmaceutically acceptable salts thereof.

Other suitable cationic lipids for use in the pharmaceutical compositions and methods of the present invention include cationic lipids as described in U.S. Provisional Patent Application Ser. No. 62/864,818, filed on Jun. 21, 2019, which is incorporated herein by reference. In some embodiments, the pharmaceutical compositions and methods of the present invention include a cationic lipid having a compound structure according to the following formula:

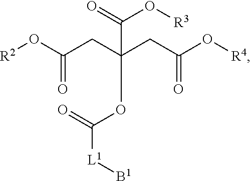

or a pharmaceutically acceptable salt thereof, wherein each of R², R³, and R⁴ is independently C₆-C₃₀ alkyl, C₆-C₃₀ alkenyl, or C₆-C₃₀ alkynyl; L¹ is C₁-C₃₀ alkylene; C₂-C₃₀ alkenylene; or C₂-C₃₀ alkynylene and B¹ is an ionizable nitrogen-containing group. In embodiments, L¹ is C₁-C₁₀ alkylene. In embodiments, L¹ is unsubstituted C₁-C₁₀ alkylene. In embodiments, L¹ is (CH₂)₂, (CH₂)₃, (CH₂)₄, or (CH₂)₅. In embodiments, L¹ is (CH₂), (CH₂)₆, (CH₂)₇, (CH₂)₈, (CH₂)₉, or (CH₂)₁₀. In embodiments, B¹ is independently NH₂, guanidine, amidine, a mono- or dialkylamine, 5- to 6-membered nitrogen-containing heterocycloalkyl, or 5- to 6-membered nitrogen-containing heteroaryl. In embodiments, B1 is

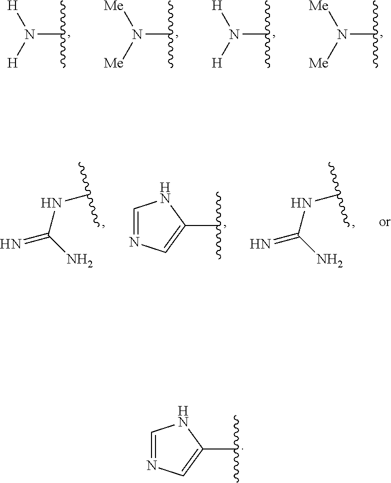

In embodiments, B¹ is

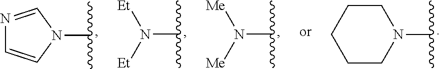

In embodiments, B¹ is

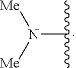 [SMILES: *N(C)C]

In embodiments, each of R², R³, and R⁴ is independently unsubstituted linear C₆-C₂₂ alkyl, unsubstituted linear C₆-C₂₂ alkenyl, unsubstituted linear C₆-C₂₂ alkynyl, unsubstituted branched C₆-C₂₂ alkyl, unsubstituted branched C₆-C₂₂ alkenyl, or unsubstituted branched C₆-C₂₂ alkynyl. In embodiments, each of R², R³, and R⁴ is unsubstituted C₆-C₂₂ alkyl. In embodiments, each of R², R³, and R⁴ is —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, —C₂₀H₄₁, —C₂₁H₄₃, —C₂₂H₄₅, —C₂₃H₄₇, —C₂₄H₄₉, or —C₂₅H₅₁. In embodiments, each of R², R³, and R⁴ is independently C₆-C₁₂ alkyl substituted by —O(CO)R⁵ or —C(O)OR⁵, wherein R⁵ is unsubstituted C₆-C₁₄ alkyl. In embodiments, each of R², R³, and R⁴ is unsubstituted C₆-C₂₂ alkenyl. In embodiments, each of R², R³, and R⁴ is —(CH₂)₄CH═CH₂, —(CH₂)₅CH═CH₂, —(CH₂)₆CH═CH₂, —(CH₂)₇CH═CH₂, —(CH₂)₈CH═CH₂, —(CH₂)₉CH═CH₂, —(CH₂)₁₀CH═CH₂, —(CH₂)₁₁CH═CH₂, —(CH₂)₁₂CH═CH₂, —(CH₂)₁₃CH═CH₂, —(CH₂)₁₄CH═CH₂, —(CH₂)₁₅CH═CH₂, —(CH₂)₁₆CH═CH₂, —(CH₂)₁₇CH═CH₂, —(CH₂)₁₈CH═CH₂, —(CH₂)₇CH═CH(CH₂)₃CH₃, —(CH₂)₇CH═CH(CH₂)₅CH₃, —(CH₂)₄CH═CH(CH₂)₈CH₃, —(CH₂)₇CH═CH(CH₂)₇CH₃, —(CH₂)₆CH═CHCH₂CH═CH(CH₂)₄CH₃, —(CH₂)₇CH═CHCH₂CH═CH(CH₂)₄CH₃, —(CH₂)₇CH═CHCH₂CH═CHCH₂CH═CHCH₂CH₃, —(CH₂)₃CH═CHCH₂CH═CHCH₂CH═CHCH₂CH═CH(CH₂)₄CH₃, —(CH₂)₃CH═CHCH₂CH═CHCH₂CH═CHCH₂CH═CHCH₂CH═CHCH₂CH₃, —(CH₂)₁₁CH═CH(CH₂)/CH₃, or —(CH₂)₂CH═CHCH₂CH═CHCH₂CH═CHCH₂CH═CHCH₂CH═CHCH₂CH═CHCH₂CH₃.
In embodiments, said C₆-C₂₂ alkenyl is a monoalkenyl, a dienyl, or a trienyl. In embodiments, each of R², R³, and R⁴ is

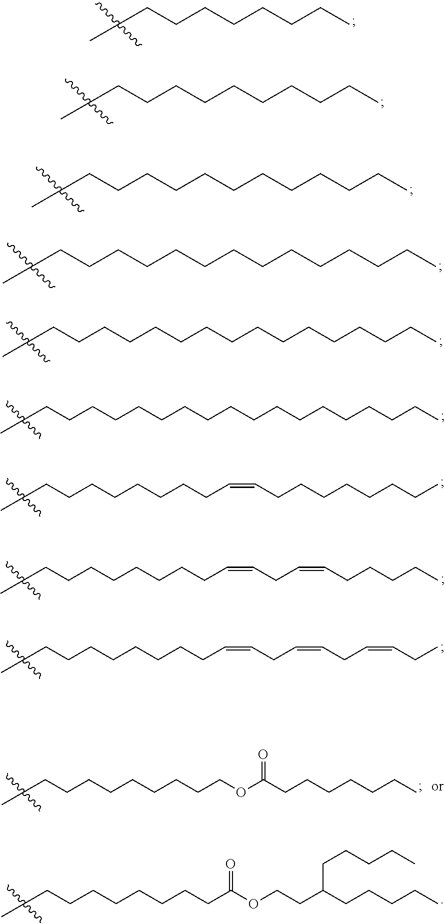

In some embodiments, the pharmaceutical compositions and methods of the present invention include a cationic lipid having a compound structure of:

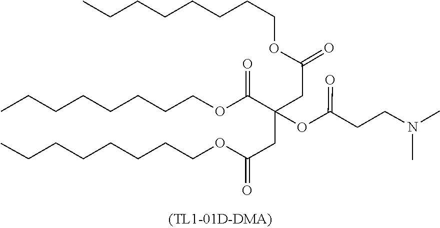 [SMILES: CCCCCCCCOC(=O)CC(CC(=O)OCCCCCCCC)(OC(=O)CCN(C)C)C(=O)OCCCCCCCC]

and pharmaceutically acceptable salts thereof. In some embodiments, the pharmaceutical compositions and methods of the present invention include a cationic lipid having a compound structure of:

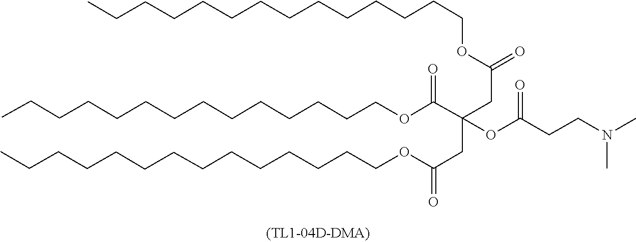 [SMILES: CCCCCCCCCCCCCCOC(=O)CC(CC(=O)OCCCCCCCCCCCCCC)(OC(=O)CCN(C)C)C(=O)OCCCCCCCCCCCCCC]

and pharmaceutically acceptable salts thereof. In some embodiments, the pharmaceutical compositions and methods of the present invention include a cationic lipid having a compound structure of:

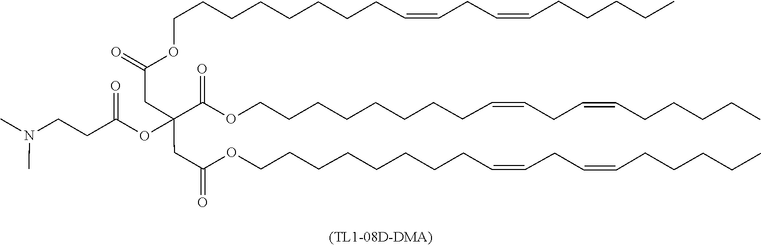 [SMILES: CCCCC/C=C\C/C=C\CCCCCCCCOC(=O)CC(CC(=O)OCCCCCCCC/C=C\C/C=C\CCCCC)(OC(=O)CCN(C)C)C(=O)OCCCCCCCC/C=C\C/C=C\CCCCC]

and pharmaceutically acceptable salts thereof. In some embodiments, the pharmaceutical compositions and methods of the present invention include a cationic lipid having a compound structure of:

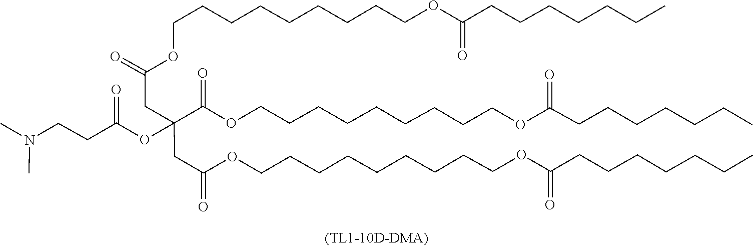 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

and pharmaceutically acceptable salts thereof.

Other suitable cationic lipids for use in the pharmaceutical compositions and methods of the present invention include cleavable cationic lipids as described in International Patent Publication WO 2012/170889, which is incorporated herein by reference. In some embodiments, the pharmaceutical compositions and methods of the present invention include a cationic lipid of the following formula:

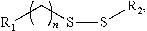

wherein R₁ is selected from the group consisting of imidazole, guanidinium, amino, imine, enamine, an optionally-substituted alkyl amino (e.g., an alkyl amino such as dimethylamino) and pyridyl; wherein R2 is selected from the group consisting of one of the following two formulas:

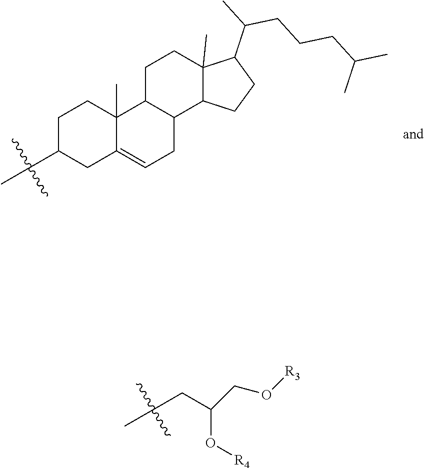

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In some embodiments, the pharmaceutical compositions and methods of the present invention include a cationic lipid, “HGT4001”, having a compound structure of:

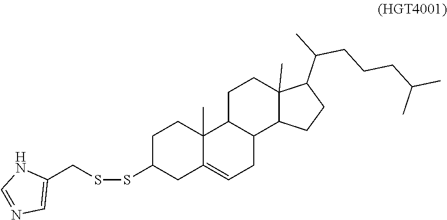 [SMILES: CC(C)CCCC(C)C1CCC2C3CC=C4CC(SSCc5cnc[nH]5)CCC4(C)C3CCC12C]

and pharmaceutically acceptable salts thereof. In some embodiments, the pharmaceutical compositions and methods of the present invention include a cationic lipid, “HGT4002”, having a compound structure of:

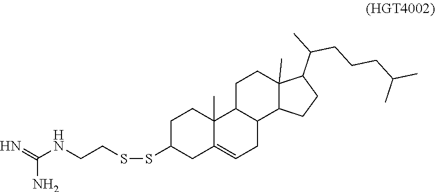 [SMILES: CC(C)CCCC(C)C1CCC2C3CC=C4CC(SSCCNC(=N)N)CCC4(C)C3CCC12C]

and pharmaceutically acceptable salts thereof. In some embodiments, the pharmaceutical compositions and methods of the present invention include a cationic lipid, “HGT4003,” having a compound structure of:

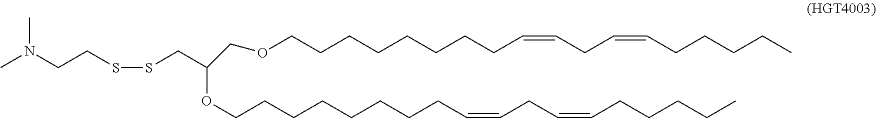 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In some embodiments, the pharmaceutical compositions and methods of the present invention include a cationic lipid, “HGT4004,” having a compound structure of:

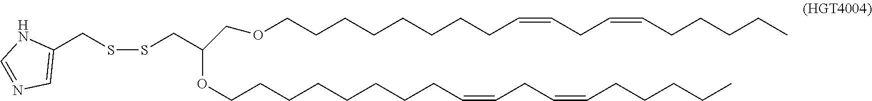 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In some embodiments, the pharmaceutical compositions and methods of the present invention include a cationic lipid “HGT4005,” having a compound structure of:

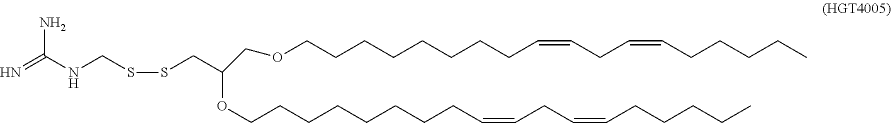 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the pharmaceutical compositions and methods of the present invention include cleavable cationic lipids as described in International Patent Publication WO 2019/222424, and incorporated herein by reference. In some embodiments, the pharmaceutical compositions and methods of the present invention include a cationic lipid that is any of general formulas or any of structures (1a) (21a) and (1b)-(21b) and (22)-(237) described in International Patent Publication WO 2019/222424. In some embodiments, the pharmaceutical compositions and methods of the present invention include a cationic lipid that has a structure according to Formula (I′),

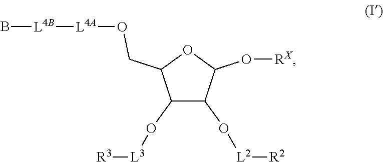

wherein:
R^{X} is independently —H, -L¹-R¹, or -L^{5A}-L^{5B}-B′;
each of L¹, L², and L³ is independently a covalent bond, —C(O)—, —C(O)O—, —C(O)S—, or —C(O)NR^{L}—;
each L^{4A} and L^{5A} is independently-C(O)—, —C(O)O—, or —C(O)NR^{L}—;
each L^{4B} and L^{5B} is independently C₁-C₂₀ alkylene; C₂-C₂₀ alkenylene; or C₂-C₂₀ alkynylene;
each B and B′ is NR⁴R⁵ or a 5- to 10-membered nitrogen-containing heteroaryl;
each R¹, R², and R³ is independently C₆-C₃₀ alkyl, C₆-C₃₀ alkenyl, or C₆-C₃₀ alkynyl;
each R⁴ and R⁵ is independently hydrogen, C₁-C₁₀ alkyl; C₂-C₁₀ alkenyl; or C₂-C₁₀ alkynyl; and
each R^{L} is independently hydrogen, C₁-C₂₀ alkyl, C₂-C₂₀ alkenyl, or C₂-C₂₀ alkynyl.
   In some embodiments, the pharmaceutical compositions and methods of the present invention include a cationic lipid that is Compound (139) of International Application No. PCT/US2019/032522, having a compound structure of:

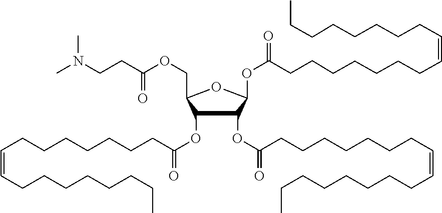

(“18:1 Carbon tail-ribose lipid”).

In some embodiments, the pharmaceutical compositions and methods of the present invention include a cationic lipid that is RL3-DMA-07D having a compound structure of:

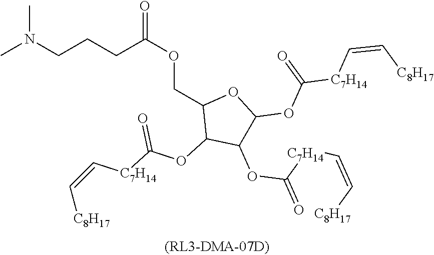

and pharmaceutically acceptable salts thereof.

In some embodiments, the pharmaceutical compositions and methods of the present invention include the cationic lipid, N-[1-(2,3-dioleyloxy) propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the pharmaceutical compositions and methods of the present invention include, for example, 5-carboxyspermylglycinedioctadecylamide (“DOGS”); 2,3-dioleyloxy-N-[2 (spermine-carboxamido) ethyl]-N,N-dimethyl-1-propanaminium (“DOSPA”) (Behr et al. Proc. Nat.′l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the pharmaceutical compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis, cis-9,12-octadecadienoxy) propane (“CLinDMA”); 2-[5′-(cholest-5-en-3-beta-oxy)-3′-oxapentoxy)-3-dimethy 1-1-(cis, cis-9′, 1-2′-octadecadienoxy) propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z, 12Z)-octadeca-9, 12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9, 12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,1 2-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28:172-176 (2010)). (Heyes, J., et al., J Controlled Release 107:276-287 (2005); Morrissey, D V., et al., Nat. Biotechnol. 23 (8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the pharmaceutical compositions and methods of the present invention include 2,2-Dilinoley 1-4-dimethylaminoethy 1-[1,3]-dioxolane (“XTC”); (3aR,5s,6aS)-N,N-dimethyl-2,2-di((9Z,12Z)-octadeca-9,12-dienyl)tetrahydro-3aH-cyclopenta [d][1,3]dioxol-5-amine (“ALNY-100”) and/or 4,7,13-tris(3-oxo-3-(undecylamino) propyl)-N1,N16-diundecyl-4,7,10,13-tetraazahexadecane-1,16-diamide (“NC98-5”).

In some embodiments, the pharmaceutical compositions of the present invention include one or more cationic lipids that constitute at least about 5%, 10%, 20%, 30%, 35%, 40%, 45%, 50%, 55%, 60%, 65%, or 70%, measured by weight, of the total lipid content in the pharmaceutical composition, e.g., a lipid nanoparticle. In some embodiments, the pharmaceutical compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the pharmaceutical composition, e.g., a lipid nanoparticle. In some embodiments, the pharmaceutical compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the pharmaceutical composition, e.g., a lipid nanoparticle. In some embodiments, the pharmaceutical compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the pharmaceutical composition, e.g., a lipid nanoparticle.

### Non-Cationic Lipids

In some embodiments, the lipid nanoparticles contain one or more non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 1,2-dierucoyl-sn-glycero-3-phosphoethanolamine (DEPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof. In some embodiments, lipid nanoparticles suitable for use with the invention include DOPE as the non-cationic lipid component. In other embodiments, lipid nanoparticles suitable for use with the invention include DEPE as the non-cationic lipid component.

In some embodiments, a non-cationic lipid is a neutral lipid, i.e., a lipid that does not carry a net charge in the conditions under which the pharmaceutical composition is formulated and/or administered.

In some embodiments, such non-cationic lipids may be used alone, but are preferably used in combination with other lipids, for example, cationic lipids.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a pharmaceutical composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a pharmaceutical composition. In some embodiments, the percentage of non-cationic lipid in a lipid nanoparticle may be greater than about 5 mol %, greater than about 10 mol %, greater than about 20 mol %, greater than about 30 mol %, or greater than about 40 mol %. In some embodiments, the percentage total non-cationic lipids in a lipid nanoparticle may be greater than about 5 mol %, greater than about 10 mol %, greater than about 20 mol %, greater than about 30 mol %, or greater than about 40 mol %. In some embodiments, the percentage of non-cationic lipid in a lipid nanoparticle is no more than about 5 mol %, no more than about 10 mol %, no more than about 20 mol %, no more than about 30 mol %, or no more than about 40 mol %. In some embodiments, the percentage total non-cationic lipids in a lipid nanoparticle may be no more than about 5 mol %, no more than about 10 mol %, no more than about 20 mol %, no more than about 30 mol %, or no more than about 40 mol %.

In some embodiments, a non-cationic lipid may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a pharmaceutical composition. In some embodiments, total non-cationic lipids may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a pharmaceutical composition. In some embodiments, the percentage of non-cationic lipid in a lipid nanoparticle may be greater than about 5 wt %, greater than about 10 wt %, greater than about 20 wt %, greater than about 30 wt %, or greater than about 40 wt %. In some embodiments, the percentage total non-cationic lipids in a lipid nanoparticle may be greater than about 5 wt %, greater than about 10 wt %, greater than about 20 wt %, greater than about 30 wt %, or greater than about 40 wt %. In some embodiments, the percentage of non-cationic lipid in a lipid nanoparticle is no more than about 5 wt %, no more than about 10 wt %, no more than about 20 wt %, no more than about 30 wt %, or no more than about 40 wt %. In some embodiments, the percentage total non-cationic lipids in a lipid nanoparticle may be no more than about 5 wt %, no more than about 10 wt %, no more than about 20 wt %, no more than about 30 wt %, or no more than about 40 wt %.

Cholesterol-Based Lipids In some embodiments, the lipid nanoparticles comprise one or more cholesterol-based lipids. For example, suitable cholesterol-based cationic lipids include, for example, DC-Choi (N,N-dimethyl-N-ethylcarboxamidocholesterol), 1,4-bis(3-N-oleylamino-propyl) piperazine (Gao, et al. Biochem. Biophys. Res. Comm. 179, 280 (1991); Wolf et al. BioTechniques 23, 139 (1997); U.S. Pat. No. 5,744,335), or imidazole cholesterol ester (ICE), as disclosed in International Patent Publication WO 2011/068810, which has the following structure:

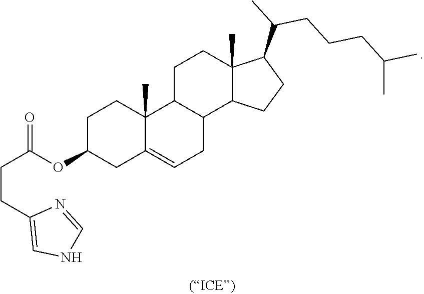 [SMILES: CC(C)CCCC(C)[C@H]1CCC2C3CC=C4C[C@@H](OC(=O)CCc5c[nH]cn5)CC[C@]4(C)C3CC[C@@]21C]

In embodiments, a cholesterol-based lipid is cholesterol.

In some embodiments, the cholesterol-based lipid may comprise a molar ratio (mol %) of about 1% to about 30%, or about 5% to about 20% of the total lipids present in a lipid nanoparticle. In some embodiments, the percentage of cholesterol-based lipid in the lipid nanoparticle may be greater than about 5 mol %, greater than about 10 mol %, greater than about 20 mol %, greater than about 30 mol %, or greater than about 40 mol %. In some embodiments, the percentage of cholesterol-based lipid in the lipid nanoparticle may be no more than about 5 mol %, no more than about 10 mol %, no more than about 20 mol %, no more than about 30 mol %, or no more than about 40 mol %.

In some embodiments, a cholesterol-based lipid may be present in a weight ratio (wt %) of about 1% to about 30%, or about 5% to about 20% of the total lipids present in a lipid nanoparticle. In some embodiments, the percentage of cholesterol-based lipid in the lipid nanoparticle may be greater than about 5 wt %, greater than about 10 wt %, greater than about 20 wt %, greater than about 30 wt %, or greater than about 40 wt %. In some embodiments, the percentage of cholesterol-based lipid in the lipid nanoparticle may be no more than about 5 wt %, no more than about 10 wt %, no more than about 20 wt %, no more than about 30 wt %, or no more than about 40 wt %.

### PEG-Modified Lipids

In some embodiments, the lipid nanoparticle comprises one or more PEGylated lipids.

For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid pharmaceutical compositions together which comprise the transfer vehicle (e.g., a lipid nanoparticle).

Contemplated PEG-modified lipids include, but are not limited to, a polyethylene glycol chain of up to 5 kDa in length covalently attached to a lipid with alkyl chain(s) of C6-C20 length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid pharmaceutical composition to the target tissues, (Klibanov et al. (1990) FEBS Letters, 268 (1): 235-237), or they may be selected to rapidly exchange out of the pharmaceutical composition in vivo (see U.S. Pat. No. 5,885,613). Particularly useful exchangeable lipids are PEG-ceramides having shorter acyl chains (e.g., C14 or C18). Lipid nanoparticles suitable for use with the invention typically include a PEG-modified lipid such as 1,2-dimyristoyl-rac-glycero-3-methoxypolyethylene glycol-2000 (DMG-PEG2K).

The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle (e.g., a lipid nanoparticle disclosed herein). In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio. For certain applications, such as pulmonary delivery, lipid nanoparticles in which the PEG-modified lipid component constitutes about 5% of the total lipids by molar ratio have been found to be particularly suitable.

### Ratio of Distinct Lipid Components

A suitable lipid nanoparticle for the present invention may include one or more of any of the cationic lipids, non-cationic lipids, cholesterol lipids, PEG-modified lipids, amphiphilic block copolymers and/or polymers described herein at various ratios. In some embodiments, a lipid nanoparticle comprises five and no more than five distinct components of nanoparticle. In some embodiments, a lipid nanoparticle comprises four and no more than four distinct components of nanoparticle. In some embodiments, a lipid nanoparticle comprises three and no more than three distinct components of nanoparticle. As non-limiting examples, a suitable lipid nanoparticle pharmaceutical composition may include a combination selected from cKK-E12, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; HGT4001, DOPE, cholesterol and DMG-PEG2K; HGT4002, DOPE, cholesterol and DMG-PEG2K; TL1-01D-DMA, DOPE, cholesterol and DMG-PEG2K; TL1-04D-DMA, DOPE, cholesterol and DMG-PEG2K; TL1-08D-DMA, DOPE, cholesterol and DMG-PEG2K; TL1-10D-DMA, DOPE, cholesterol and DMG-PEG2K; ICE, DOPE and DMG-PEG2K; HGT4001, DOPE and DMG-PEG2K; or HGT4002, DOPE and DMG-PEG2K.

In various embodiments, cationic lipids (e.g., cKK-E12, C12-200, TL1-01D-DMA, TL1-04D-DMA, TL1-08D-DMA, TL1-10D-DMA, ICE, HGT4001, HGT4002 and/or HGT4003) constitute about 30-60% (e.g., about 30-55%, about 30-50%, about 30-45%, about 30-40%, about 35-50%, about 35-45%, or about 35-40%) of the lipid nanoparticle by molar ratio. In some embodiments, the percentage of cationic lipids (e.g., cKK-E12, C12-200, TL1-01D-DMA, TL1-04D-DMA, TL1-08D-DMA, TL1-10D-DMA, ICE, HGT4001, HGT4002 and/or HGT4003) is or greater than about 30%, about 35%, about 40%, about 45%, about 50%, about 55%, or about 60% of the lipid nanoparticle by molar ratio.

In some embodiments, the molar ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

In embodiments where a lipid nanoparticle comprises three and no more than three distinct components of lipids, the ratio of total lipid content (i.e., the ratio of lipid component (1):lipid component (2):lipid component (3)) can be represented as x:y:z, wherein

$$( y + z ) = 100 - x .$$

In some embodiments, each of “x,” “y,” and “z” represents molar percentages of the three distinct components of lipids, and the ratio is a molar ratio.

In some embodiments, each of “x,” “y,” and “z” represents weight percentages of the three distinct components of lipids, and the ratio is a weight ratio.

In some embodiments, lipid component (1), represented by variable “x,” is a sterol-based cationic lipid.

In some embodiments, lipid component (2), represented by variable “y,” is a non-cationic lipid.

In some embodiments, lipid component (3), represented by variable “z” is a PEG lipid.

In some embodiments, variable “x,” representing the molar percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the molar percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the molar percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “x,” representing the weight percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the weight percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the weight percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “z,” representing the molar percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the molar percentage of lipid component (3) (e.g., a PEG lipid) is about 1%, 2%, 3%, 4%, 5%, 6%, 7%, 8%, 9%, 10%. In embodiments, variable “z,” representing the molar percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

In some embodiments, variable “z,” representing the weight percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the weight percentage of lipid component (3) (e.g., a PEG lipid) is about 1%, 2%, 3%, 4%, 5%, 6%, 7%, 8%, 9%, 10%. In embodiments, variable “z,” representing the weight percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

For pharmaceutical compositions having three and only three distinct lipid components, variables “x,” “y,” and “z” may be in any combination so long as the total of the three variables sums to 100% of the total lipid content. For example, in typical three-component lipid nanoparticles suitable for use with the invention, the molar ratio of cationic lipid to non-cationic lipid to PEG-modified lipid may be between about 55-65:30-40:1-15, respectively. In some embodiments, a molar ratio of cationic lipid (e.g., a sterol-based lipid) to non-cationic lipid (e.g., DOPE or DEPE) to PEG-modified lipid (e.g., DMG-PEG2K) of 60:35:5 is particularly suitable, e.g., for pulmonary delivery of lipid nanoparticles via nebulization.

### Exemplary Lipid Nanoparticle Formulation

An exemplary lipid nanoparticle for in vivo delivery of a nucleic acids in accordance with the present invention comprises a cationic lipid (e.g., cKK-E10), a non-cationic lipid (e.g., DOPE), cholesterol and a PEG-modified lipid (e.g., DMG-PEG2K). In a particular embodiment, the invention provides a lipid nanoparticle for the delivery of the nucleic acids of the invention, which has a lipid component consisting of cKK-E10, DOPE, cholesterol and DMG-PEG2K at the molar ratios 40:30:28.5:1.5. As shown in the examples, this lipid nanoparticle formulation has been found to be particularly effective for use in the immunogenic compositions of the invention, in particular for intramuscular administration of lipid nanoparticles comprising the nucleic acids of the invention.

### Lipid Nanoparticle Compositions Containing at Least Two Nucleic Acids

In some embodiments, at least two nucleic acids comprising different optimized nucleotide sequences of the invention are encapsulated in the same lipid nanoparticle (e.g., a lipid nanoparticle comprising cKK-E10, DOPE, cholesterol and DMG-PEG2K). For example, a first nucleic acid (e.g., an mRNA) comprising a first optimized nucleotide sequence of the invention may be combined with a second nucleic acid (e.g., an mRNA) comprising a second optimized nucleotide sequence of the invention and encapsulated in the same lipid nanoparticle.

In other embodiments, at least two nucleic acids comprising different optimized nucleotide sequences of the invention are encapsulated separately (typically using a lipid nanoparticle formulation having the same lipid composition, e.g., cKK-E10, DOPE, cholesterol and DMG-PEG2K). For example, a first nucleic acid (e.g., an mRNA) comprising a first optimized nucleotide sequence of the invention and a second nucleic acid (e.g., an mRNA) comprising a second optimized nucleotide sequence of the invention may each be encapsulated in separate lipid nanoparticles, which are then combined to provide a mixture of lipid nanoparticles encapsulating the first nucleic acid and lipid nanoparticles encapsulating the second nucleic acid (typically at a 1:1 ratio).

For instance, an immunogenic composition in accordance with the invention may comprise at least two nucleic acids, wherein the first nucleic acid comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline (e.g., an mRNA comprising the optimized nucleotide sequence of SEQ ID NO: 44, or the exemplary mRNA construct 1 shown in Table 4); and the second nucleic acid comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline and further contains the L18F, D80A, D215G, L242-, A243-, L244-, K417N, E484K, N501Y, D614G and A701V mutations (e.g., an mRNA comprising the optimized nucleotide sequence of SEQ ID NO: 166, or the exemplary mRNA construct 2 shown in Table 4). In some embodiments, the first nucleic acid may be combined with the second nucleic acid and encapsulated in the same lipid nanoparticle. In other embodiments, the first nucleic acid and the second nucleic acid may each be encapsulated in separate lipid nanoparticles (typically formed from the same lipid components, e.g., cKK-E10, DOPE, cholesterol and DMG-PEG2K). The lipid nanoparticles encapsulating the first nucleic acid and the lipid nanoparticles encapsulating the second nucleic acid are then combined (typically at a 1:1 ratio).

### Pharmaceutical Compositions

A nucleic acid comprising an optimized nucleotide sequence encoding a SARS-COV-2 antigen in accordance with the invention may be provided in a pharmaceutical composition (e.g., an immunogenic composition or a vaccine). In a typical embodiment, a pharmaceutical composition in accordance with the invention comprises a nucleic acid in accordance with the invention and a lipid nanoparticle. In particular embodiments, the nucleic acid is encapsulated in the lipid nanoparticle. In some embodiments, the lipid nanoparticle may comprise one or more of a cationic lipid, a non-cationic lipid, a cholesterol-based lipid, a PEG-modified lipid, or a combination thereof. In a typical embodiment, the lipid nanoparticle comprises a cationic lipid, a non-cationic lipid, a cholesterol-based lipid, and a PEG-modified lipid. In some embodiments, the lipid nanoparticle comprises a cationic lipid, a non-cationic lipid, and a PEG-modified lipid.

### Pharmaceutically Acceptable Excipients

To stabilize the nucleic acid and/or lipid nanoparticle, or to facilitate administration of the pharmaceutical composition and/or enhance in vivo expression of the nucleic acids of the invention, the nucleic acid and/or lipid nanoparticle can be formulated in combination with one or more additional nucleic acids, carriers, targeting ligands, stabilizing reagents, and/or other pharmaceutically acceptable excipients. Techniques for formulation and administration of drugs may be found in “Remington's Pharmaceutical Sciences,” Mack Publishing Co., Easton, Pa., latest edition.

In some embodiments, the pharmaceuticals composition is formulated with a diluent. In some embodiments, the diluent is selected from a group consisting of DMSO, ethylene glycol, glycerol, 2-Methyl-2,4-pentanediol (MPD), propylene glycol, sucrose, and trehalose. In some embodiments, the formulation comprises 1%, 2%, 3%, 4%, 5%, 6%, 7%, 8%, 9%, 10%, 11%, 12%, 13%, 14%, 15%, 16%, 17%, 18%, 19% or 20% diluent. In a particular embodiment, the mRNA is formulated in 10% trehalose as the diluent.

### Therapeutically Effective Amount

The nucleic acid in accordance with the invention is provided in a therapeutically effective amount in the pharmaceutical compositions provided here. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating or preventing an infection with a SARS-COV-2 infection). For example, a therapeutically effective amount may be an amount sufficient to achieve a desired prophylactic effect with an immunogenic composition of the invention.

In some embodiments, a pharmaceutical composition (e.g., an immunogenic composition) in accordance with the present invention comprises an mRNA comprising an optimized nucleotide sequence encoding a SARS-COV-2 antigen at a concentration ranging from 0.1 mg/mL to 10.0 mg/mL. In some embodiments, the mRNA is at a concentration of at least 0.1 mg/mL. In some embodiments, the mRNA is at a concentration of at least 0.2 mg/mL. In some embodiments, the mRNA is at a concentration of at least 0.3 mg/mL. In some embodiments, the mRNA is at a concentration of at least 0.4 mg/mL. In some embodiments, the mRNA is at a concentration of at least 0.5 mg/mL. In some embodiments, the mRNA is at a concentration of at least 0.6 mg/mL. In some embodiments, the mRNA is at a concentration of at least 0.7 mg/mL. In some embodiments, the mRNA is at a concentration of at least 0.8 mg/mL. In some embodiments, the mRNA is at a concentration of at least 0.9 mg/mL. In some embodiments, the mRNA is at a concentration of at least 1.0 mg/mL. In a typical embodiment, the mRNA is at a concentration of about 0.5 mg/mL to about 1.0 mg/mL, e.g., about 0.6 mg/mL to about 0.8 mg/mL.

In some embodiments, a pharmaceutical composition (e.g., an immunogenic composition) in accordance with the present invention comprises an mRNA comprising an optimized nucleotide sequence encoding a SARS-COV-2 antigen at a dose of between 5 μg and 200 μg. In some embodiments, the mRNA dose in the pharmaceutical composition is 10 μg and 200 μg. In some embodiments, the mRNA dose in the pharmaceutical composition is between 7 μg and 135 μg. In particular embodiments, the mRNA dose in the pharmaceutical composition is between 15 μg and 135 μg (e.g., between 15 μg and 45 μg).

In some embodiments, the mRNA dose in the pharmaceutical composition is at least 5 μg. In some embodiments, the mRNA dose in the pharmaceutical composition is at least 10 μg. In some embodiments, the mRNA dose in the pharmaceutical composition is at least 15 μg. In some embodiments, the mRNA dose in the pharmaceutical composition is at least 20 μg. In some embodiments, the mRNA dose in the pharmaceutical composition is at least 25 μg. In some embodiments, the mRNA dose in the pharmaceutical composition is at least 30 μg. In some embodiments, the mRNA dose in the pharmaceutical composition is at least 35 μg. In some embodiments, the mRNA dose in the pharmaceutical composition is at least 40 μg. In some embodiments, the mRNA dose in the pharmaceutical composition is at least 45 μg. In some embodiments, the mRNA dose in the pharmaceutical composition is at least 50 μg. In some embodiments, the mRNA dose in the pharmaceutical composition is at least 75 μg. In some embodiments, the mRNA dose in the pharmaceutical composition is at least 100 μg. In some embodiments, the mRNA dose in the pharmaceutical composition is at least 150 μg.

In a specific embodiment, the mRNA dose in the pharmaceutical composition is about 7.5 μg. In another specific embodiment, the mRNA dose in the pharmaceutical composition is about 10 μg. In another specific embodiment, the mRNA dose in the pharmaceutical composition is about 15 μg. In another specific embodiment, the mRNA dose in the pharmaceutical composition is about 20 μg. In another specific embodiment, the mRNA dose in the pharmaceutical composition is about 30 μg. In another specific embodiment, the mRNA dose in the pharmaceutical composition is about 40 μg. In another specific embodiment, the mRNA dose in the pharmaceutical composition is about 45 μg. In another specific embodiment, the mRNA dose in the pharmaceutical composition is about 135 μg.

In some embodiments, a pharmaceutical composition (e.g., an immunogenic composition) in accordance with the present invention comprises more than one mRNA construct (e.g., at least two mRNA constructs) comprising an optimized nucleotide sequence encoding a SARS-COV-2 antigen (e.g., two mRNA constructs encoding naturally occurring variants of the SARS-COV-2 S protein). Accordingly, in some embodiments, the total dose of the mRNA constructs is 5 μg and 200 μg. For example, the total dose of the mRNA constructs is between 10 μg and 200 μg. In some embodiments, the total dose of the mRNA constructs is between 7 μg and 135 μg. In particular embodiments, the total dose of the mRNA constructs is between 15 μg and 135 μg (e.g., between 15 μg and 45 μg).

In some embodiments, the total dose of the mRNA constructs is at least 5 μg. In some embodiments, the total dose of the mRNA constructs is at least 10 μg. In some embodiments, the total dose of the mRNA constructs is at least 15 μg. In some embodiments, the total dose of the mRNA constructs is at least 20 μg. In some embodiments, the total dose of the mRNA constructs is at least 25 μg. In some embodiments, the total dose of the mRNA constructs is at least 30 μg. In some embodiments, the total dose of the mRNA constructs is at least 35 μg. In some embodiments, the total dose of the mRNA constructs is at least 40 μg. In some embodiments, the total dose of the mRNA constructs is at least 45 μg. In some embodiments, the total dose of the mRNA constructs is at least 50 μg. In some embodiments, the total dose of the mRNA constructs is at least 75 μg. In some embodiments, the total dose of the mRNA constructs is at least 100 μg. In some embodiments, the total dose of the mRNA constructs is at least 150 μg.

In a specific embodiment, the total dose of the mRNA constructs in the pharmaceutical composition is about 7.5 μg. In another specific embodiment, the total dose of the mRNA constructs in the pharmaceutical composition is about 10 μg. In another specific embodiment, the total dose of the mRNA constructs in the pharmaceutical composition is about 15 μg. In another specific embodiment, the total dose of the mRNA constructs in the pharmaceutical composition is about 20 μg. In another specific embodiment, the total dose of the mRNA constructs in the pharmaceutical composition is about 30 μg. In another specific embodiment, the total dose of the mRNA constructs in the pharmaceutical composition is about 40 μg. In another specific embodiment, the total dose of the mRNA constructs in the pharmaceutical composition is about 45 μg. In another specific embodiment, the total dose of the mRNA constructs in the pharmaceutical composition is about 135 μg.

### Combinations of SARS-COV-2 S Proteins

In some embodiments, an immunogenic composition in accordance with the invention comprises more than one optimized nucleotide sequence encoding a SARS-COV-2 spike protein. In some embodiments, each of the optimized nucleotide sequences encodes a naturally occurring variant of a SARS-COV-2 spike protein. In some embodiments, one or more of these optimized nucleotide sequences encodes a SARS-COV-2 spike protein that has been modified relative to naturally occurring SARS-COV-2 spike protein. In particular embodiments, the modifications stabilize the SARS-COV-2 spike protein in its prefusion conformation, as described in detail above.

In some embodiments, an immunogenic composition in accordance with the present invention comprises at least two nucleic acids, for use in prophylaxis of an infection with SARS-CoV-2, wherein a first nucleic acid comprises an optimized nucleotide sequence which encodes an amino acid sequence comprising a sequence selected from SEQ ID NO: 1, 2, 3, 4, 5, 8, 9, 10, 11, 12, 14, 15, 16, 17, 19, 20, 35, 66, 68, 70, 72, 74, 76, 78, 80, 82, 84. 86, 88, 90, 92, 94, 96, 98, 104, 106, 108, 110, 118, 120, 123, 125, 127, 129, 131, 133, 135, 137, 139 or 141, and wherein one or more further nucleic acid(s) comprise(s) an optimized nucleotide sequence which encodes an amino acid sequence comprising a sequence selected from SEQ ID NO: 151, 153, 155, 157, 159, 161, 163, 165, 167, 169 or 171.

In some embodiments, an immunogenic composition in accordance with the present invention comprises at least two nucleic acids, for use in prophylaxis of an infection with SARS-CoV-2, wherein a first nucleic acid that encodes an amino acid sequence comprising SEQ ID NO: 11; and wherein a second nucleic acid comprises an optimized nucleotide sequence that encodes an amino acid sequence of SEQ ID NO: 157.

In some embodiments, an immunogenic composition in accordance with the present invention comprises at least two nucleic acids, for use in prophylaxis of an infection with SARS-CoV-2, wherein a first nucleic acid comprises an optimized nucleotide sequence that is at least 85% (e.g., at least 90%) identical to the nucleic acid sequence of SEQ ID NO: 44 and encodes an amino acid sequence comprising SEQ ID NO:11, optionally wherein the optimized nucleotide sequence has the nucleic acid sequence of SEQ ID NO: 44; and wherein a second nucleic acid comprises an optimized nucleotide sequence that is at least 85% (e.g., at least 90%) identical to the nucleic acid sequence of SEQ ID NO: 156 and encodes an amino acid sequence comprising SEQ ID NO: 157, optionally wherein the optimized nucleotide sequence has the nucleic acid sequence of SEQ ID NO 156.

In some embodiments, an immunogenic composition in accordance with the present invention comprises at least two nucleic acids, for use in prophylaxis of an infection with SARS-CoV-2, wherein a first nucleic acid that encodes an amino acid sequence comprising SEQ ID NO: 11; and wherein a second nucleic acid comprises an optimized nucleotide sequence that encodes an amino acid sequence of SEQ ID NO: 163.

In some embodiments, an immunogenic composition in accordance with the present invention comprises at least two nucleic acids, for use in prophylaxis of an infection with SARS-CoV-2, wherein a first nucleic acid comprises an optimized nucleotide sequence that is at least 85% (e.g., at least 90%) identical to the nucleic acid sequence of SEQ ID NO: 44 and encodes an amino acid sequence comprising SEQ ID NO:11, optionally wherein the optimized nucleotide sequence has the nucleic acid sequence of SEQ ID NO: 44; and wherein a second nucleic acid comprises an optimized nucleotide sequence that is at least 85% (e.g., at least 90%) identical to the nucleic acid sequence of SEQ ID NO: 162 and encodes an amino acid sequence comprising SEQ ID NO: 163, optionally wherein the optimized nucleotide sequence has the nucleic acid sequence of SEQ ID NO 162.

In some embodiments, an immunogenic composition in accordance with the present invention comprises at least two nucleic acids, for use in prophylaxis of an infection with SARS-CoV-2, wherein a first nucleic acid that encodes an amino acid sequence comprising SEQ ID NO: 11; and wherein a second nucleic acid comprises an optimized nucleotide sequence that encodes an amino acid sequence of SEQ ID NO: 167.

In some embodiments, an immunogenic composition in accordance with the present invention comprises at least two nucleic acids, for use in prophylaxis of an infection with SARS-CoV-2, wherein a first nucleic acid comprises an optimized nucleotide sequence that is at least 85% (e.g., at least 90%) identical to the nucleic acid sequence of SEQ ID NO: 44 and encodes an amino acid sequence comprising SEQ ID NO:11, optionally wherein the optimized nucleotide sequence has the nucleic acid sequence of SEQ ID NO: 44; and wherein a second nucleic acid comprises an optimized nucleotide sequence that is at least 85% (e.g., at least 90%) identical to the nucleic acid sequence of SEQ ID NO: 166 and encodes an amino acid sequence comprising SEQ ID NO: 167, optionally wherein the optimized nucleotide sequence has the nucleic acid sequence of SEQ ID NO 166.

In some embodiments, an immunogenic composition in accordance with the present invention comprises at least two nucleic acids, for use in prophylaxis of an infection with SARS-CoV-2, wherein a first nucleic acid that encodes an amino acid sequence comprising SEQ ID NO: 11; and wherein a second nucleic acid comprises an optimized nucleotide sequence that encodes an amino acid sequence of SEQ ID NO: 171.

In some embodiments, an immunogenic composition in accordance with the present invention comprises at least two nucleic acids, for use in prophylaxis of an infection with SARS-CoV-2, wherein a first nucleic acid comprises an optimized nucleotide sequence that is at least 85% (e.g., at least 90%) identical to the nucleic acid sequence of SEQ ID NO: 44 and encodes an amino acid sequence comprising SEQ ID NO:11, optionally wherein the optimized nucleotide sequence has the nucleic acid sequence of SEQ ID NO: 44; and wherein a second nucleic acid comprises an optimized nucleotide sequence that is at least 85% (e.g., at least 90%) identical to the nucleic acid sequence of SEQ ID NO: 170 and encodes an amino acid sequence comprising SEQ ID NO: 171, optionally wherein the optimized nucleotide sequence has the nucleic acid sequence of SEQ ID NO 170.

In some embodiments, an immunogenic composition in accordance with the present invention comprises at least three, at least four or at least five nucleic acids, for use in prophylaxis of an infection with SARS-COV-2. The first, second, third, fourth and fifth nucleic acids, as applicable, may be encapsulated in the same lipid nanoparticles. Alternatively, the first, second, third, fourth and fifth nucleic acids, as applicable, may be encapsulated in separate lipid nanoparticles which are mixed together to form a pharmaceutical composition in accordance with the present invention.

### Combinations of SARS-COV-2 Antigens

In some embodiments, a pharmaceutical composition in accordance with the invention comprises more than one optimized nucleotide sequence encoding a SARS-COV-2 antigen. In some embodiments, a pharmaceutical composition may comprise a first nucleic acid comprising an optimized nucleotide sequence encoding a SARS-COV-2 S protein or an antigenic fragment thereof and a second nucleic acid comprising an optimized nucleotide sequence encoding a SARS-CoV-2 M protein or an antigenic fragment thereof. In some embodiments, a pharmaceutical composition may comprise a first nucleic acid comprising an optimized nucleotide sequence encoding a SARS-COV-2 S protein or an antigenic fragment thereof and a second nucleic acid comprising an optimized nucleotide sequence encoding a SARS-COV-2 N protein or an antigenic fragment thereof. In some embodiments, a pharmaceutical composition may comprise a first nucleic acid comprising an optimized nucleotide sequence encoding a SARS-COV-2 S protein or an antigenic fragment thereof and a second nucleic acid comprising an optimized nucleotide sequence encoding a SARS-COV-2 E protein or an antigenic fragment thereof. In other embodiments, a pharmaceutical composition may comprise a first nucleic acid comprising an optimized nucleotide sequence encoding a SARS-COV-2 S protein or an antigenic fragment thereof and second, third and/or fourth nucleic acids, wherein said second nucleic acid comprises an optimized nucleotide sequence encoding a SARS-COV-2 M protein or an antigenic fragment thereof, wherein said third nucleic acid comprises an optimized nucleotide sequence encoding a SARS-COV-2 N protein or an antigenic fragment thereof, and wherein said fourth nucleic acid comprises an optimized nucleotide sequence encoding a SARS-COV-2 E protein or an antigenic fragment thereof.

The first, second, third and fourth nucleic acids, as applicable, may be encapsulated in the same lipid nanoparticles. Alternatively, the first, second, third and fourth nucleic acids, as applicable, may be encapsulated in separate lipid nanoparticles which are mixed together to form a pharmaceutical composition in accordance with the present invention.

### Administration

Typically, a pharmaceutical composition in accordance with the invention (e.g., an immunogenic composition or a vaccine) is administered parenterally, e.g., by an intravenous, intradermal, subcutaneous, or intramuscular route. Most commonly the administration is intramuscular. Administration may be by injection, e.g., by needle-free and/or needle injection.

For example, using lipid nanoparticles containing the cationic lipid OF-Deg-Lin, Fenton et al. (Adv Mater. 2017; 29 (33)) were able to deliver encapsulated mRNA successfully to the spleen via intravenous injection. They observed that more than 85% of total protein production occurred in the spleen. When they analyzed the spleen of test animals, they found that lipid nanoparticles delivered the encapsulated mRNA primarily to B cell and monocyte/macrophage populations. A small percentage of the mRNA also appeared to be delivered to the neutrophil and T cell populations. As shown in the examples of the present specification, pharmaceutical compositions comprising lipid nanoparticles which have a lipid component consisting of cKK-E10, DOPE, cholesterol and DMG-PEG2K at the molar ratios 40:30:28.5:1.5 are especially effective in eliciting an immune response against the encapsulated nucleic acid(s), in particular when administered intramuscularly.

### Prime-Boost Immunization

In some embodiments, a pharmaceutical composition in accordance with the invention is administered once. In some embodiments, a pharmaceutical composition in accordance with the invention is administered at least twice.

For example, a typical prime-boost immunization of a subject who has not previously been immunized against an infection with a β-coronavirus, e.g., SARS-COV-2, typically comprises at least two immunizations. Commonly, these two immunization are administered at an interval. Accordingly, in some embodiments, a pharmaceutical composition in accordance with the invention is administered at least twice (e.g., three times) at an interval of 2, 3, 4, 5, 6, 7 or 8 weeks. In some embodiments, a pharmaceutical composition in accordance with the invention is administered twice at an interval of 2, 3, 4, 5, 6, 7, 8, 9, 10, 11 or 12 weeks. In typical embodiments, the administration interval is 2 weeks or 4 weeks (e.g., 1 month). In other embodiments, the administration interval is 11 weeks, or 12 weeks (e.g. about 3 months). Accordingly, in one embodiment, the invention provides a method of preventing an infection caused by a β-coronavirus (e.g., SARS-COV-2), wherein said method comprises administering to a subject a first dose of an immunogenic composition comprising an mRNA construct of the invention, and a second dose of an immunogenic composition of the invention, wherein said first and second doses are administered at least 2 weeks apart from each other. In some embodiments, the invention provides a method of preventing an infection caused by a β-coronavirus (e.g., SARS-CoV-2), wherein said method comprises administering to a subject a first dose of an immunogenic composition comprising an mRNA construct of the invention, and a second dose of an immunogenic composition of the invention, wherein said first and second doses are administered about 3 weeks apart from each other.

Sometimes, an initial prime-boost immunization is followed by at least one further immunization to refresh the protective effective of the initial immunization series. This further immunization typically takes place several months, and sometimes several years, after the initial prime-boost immunization. Accordingly, in some embodiments, a pharmaceutical composition in accordance with the invention is administered to a subject at least once 3-18 months (e.g., about 9 months or about 12 months) after the subject was administered with at least one dose of an immunogenic composition for the prophylaxis of an infection with a β-coronavirus, e.g. a β-coronavirus expressing a spike protein which binds to angiotensin-converting enzyme 2 (ACE2), such as SARS-COV-2. For example, a subject may have received at least one dose of an immunogenic composition for the prophylaxis of an infection with a β-coronavirus (e.g., SARS-CoV-2), and 3-18 months (e.g., about 9 months or about 12 months) later, the subject is administered a pharmaceutical composition of the invention. More typically, a subject may have received two doses of an immunogenic composition for the prophylaxis of an infection with a β-coronavirus (e.g., SARS-COV-2), e.g. a first dose and, at least two weeks later, a second dose. 3-18 months after having received the second dose, the subject may be administered with a pharmaceutical composition of the invention. The administration of a pharmaceutical composition of the invention may commonly occur at least 9 months (e.g., about 12 months) after the subject has received the second dose of an immunogenic composition for the prophylaxis of an infection with a β-coronavirus (e.g., SARS-COV-2).

In some embodiments, the first and second doses may be an immunogenic composition for the prophylaxis of an infection with a β-coronavirus (e.g., SARS-COV-2), e.g., a vaccine that elicits neutralizing antibodies against the S protein of the SARS-COV-2 index strain from Wuhan (SEQ ID NO: 1). For example, the vaccine may comprise a nucleic acid encoding a full-length SARS-COV-2 spike protein which has been modified relative to naturally occurring full-length SARS-COV-2 spike protein of SEQ ID NO: 1 to mutate residues 986 and 987 to proline to stabilize the full-length SARS-COV-2 spike protein in its prefusion conformation. Vaccines that elicit neutralizing antibodies include a pharmaceutical compositions disclosed herein (e.g., an immunogenic composition or a vaccine disclosed herein) as well as COVID-19 vaccines produced by Moderna (COVID-19 Vaccine Moderna, such as for example, mRNA-1273 or mRNA-1283), CureVac (CVnCOV), Johnson & Johnson (COVID-19 Vaccine Janssen), AstraZeneca (Vaxzevria), Pfizer/BioNTech (Comirnaty), Sputnik (Gam-COVID-Vac), Sinovac (COVID-19 Vaccine (Vero Cell) Inactivated) and Novavax (NVX-CoV2373). The first dose and the second dose may comprise the same vaccine. The first dose and the second dose may comprise different vaccines.

In a particular embodiment, the pharmaceutical composition of the invention which is administered 3-18 months later comprises a nucleic acid (e.g., an mRNA) comprising an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline and further contains the L18F, D80A, D215G, L242-, A243-, L244-, K417N, E484K, N501Y, D614G and A701V mutations. In a particular embodiment, the nucleic acid (e.g., an mRNA) comprising the optimized nucleotide sequence is capable of eliciting a broadly neutralizing antibody response against naturally occurring variants of SARS-COV-2, including the Wuhan index strain as well as variants observed in South Africa, Japan, Brazil, the UK, India and California. In some embodiments, the nucleic acid (e.g., an mRNA) comprising the optimized nucleotide sequence is capable of eliciting a neutralizing antibody response against SARS-COV-1. In a specific embodiment, the nucleic acid (e.g., an mRNA) comprising the optimized nucleotide sequence is capable of eliciting a neutralizing antibody response to a β-coronavirus expressing a spike protein which binds to angiotensin-converting enzyme 2 (ACE2). In some embodiments, the spike protein is at least 75% (e.g., at least 80%, 90%, 95% or 99%) identical to SEQ ID NO: 1. In a specific embodiment, the nucleic acid (e.g., the mRNA) comprises an optimized nucleotide sequence that encodes an amino acid sequence comprising SEQ ID NO: 167, optionally wherein the optimized nucleotide sequence has the nucleic acid sequence of SEQ ID NO: 166. For example, the optimized nucleotide sequence of the mRNA may have the nucleic acid sequence of SEQ ID NO: 173.

In one specific embodiment, the pharmaceutical composition of the invention which is administered 3-18 months later comprises at least two nucleic acids (e.g., a first mRNA and a second mRNA), wherein the first nucleic acid (e.g., the first mRNA) comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline; and the second nucleic acid (e.g., the second mRNA) comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline and further contains the L18F, D80A, D215G, L242-, A243-, L244-, K417N, E484K, N501Y, D614G and A701V mutations. In a particular embodiment, the pharmaceutical composition comprising the first and second mRNAs is capable of eliciting a broadly neutralizing antibody response against naturally occurring variants of SARS-COV-2, including the Wuhan index strain as well as variants observed in South Africa, Japan, Brazil, the UK, India and California. In some embodiments, the pharmaceutical composition comprising the first and second mRNAs is capable of eliciting a neutralizing antibody response against SARS-CoV-1. In a specific embodiment, the pharmaceutical composition comprising the first and second mRNAs is capable of eliciting a neutralizing antibody response to a β-coronavirus expressing a spike protein which binds to angiotensin-converting enzyme 2 (ACE2). In some embodiments, the spike protein is at least 75% (e.g., at least 80%, 90%, 95% or 99%) identical to SEQ ID NO: 1. The first nucleic acid may comprise an optimized nucleotide sequence which encodes an amino acid sequence comprising SEQ ID NO:11, optionally wherein the optimized nucleotide sequence has the nucleic acid sequence of SEQ ID NO: 44. The second nucleic acid comprises an optimized nucleotide sequence that encodes an amino acid sequence comprising SEQ ID NO: 167, optionally wherein the optimized nucleotide sequence has the nucleic acid sequence of SEQ ID NO: 166. For example, the optimized nucleotide sequence of the first mRNA may have the nucleic acid sequence of SEQ ID NO: 148, wherein the optimized nucleotide sequence of the second mRNA may have the nucleic acid sequence of SEQ ID NO: 173. Typically, the at least two nucleic acids are encapsulated in lipid nanoparticles. For example, the first nucleic acid and the second nucleic acid may be encapsulated in the same lipid nanoparticle. Alternatively, the first nucleic acid and the second nucleic may be encapsulated in separate lipid nanoparticles.

As shown in the examples, subjects who have previously been immunized with a vaccine that elicits neutralizing antibodies against the S protein of the SARS-COV-2 index strain from Wuhan (SEQ ID NO: 1) and who are administered about 9 months later an mRNA vaccine comprising an optimized nucleotide sequence of the invention that encodes a prefusion stabilized South African variant of the SARS-COV-2 S protein are able to mount a broadly neutralizing antibody response effective against a wide variety of S proteins expressed by naturally occurring variants of the original SARS-COV-2 Wuhan strain as well as other β-coronaviruses, in particular those expressing a spike protein which binds to angiotensin-converting enzyme 2 (ACE2), such as SARS-COV-1.

Accordingly, in some embodiments, the pharmaceutical compositions of the invention are for use in the prophylaxis of an infection caused by a β-coronavirus, in particular a β-coronavirus expressing a spike protein which binds to angiotensin-converting enzyme 2 (ACE2). In some embodiments, the pharmaceutical compositions of the invention are for use in the manufacture of a medicament for the prophylaxis of an infection caused by a β-coronavirus, in particular a β-coronavirus expressing a spike protein which binds to angiotensin-converting enzyme 2 (ACE2). In some embodiments, the spike protein is at least 75% (e.g., at least 80%, 90%, 95% or 99%) identical to SEQ ID NO: 1. In a typical embodiment, the β-coronavirus is SARS-COV-2 (e.g., a naturally occurring variant of the Wuhan index strain, such as a South Africa variant, a Japanese variant, a Brazilian variant, a UK variant, an Indian variant or a California variant).

In a specific embodiment, the invention provides a method of preventing an infection caused by SARS-COV-2, wherein said method comprises administering to a subject an effective amount of an immunogenic composition comprising an mRNA construct, wherein said mRNA construct comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline and further contains the L18F, D80A, D215G, L242-, A243-, L244-, K417N, E484K, N501Y, D614G and A701V mutations, wherein said immunogenic composition is administered to the subject at least 3 months (e.g., about 6 months, about 9 months or about 12 months) after the subject was immunized with a first COVID-19 vaccine and a second COVID-19 vaccine, wherein said first and second COVID-19 vaccines were administered to the subject at least two weeks apart from each other and wherein said first and second COVID-19 vaccines were designed to elicit neutralizing antibodies against the S protein of SARS-COV-2, e.g., the S-protein of the SARS-COV-2 index strain from Wuhan (SEQ ID NO: 1). In some embodiments, the first and second COVID-19 vaccines are identical. In other embodiments, said first and second vaccines are different. In particular embodiments, said first and second COVID-19 vaccines are produced by Moderna (COVID-19 Vaccine Moderna, such as for example, mRNA-1273 or mRNA-1283), CureVac (CVnCOV), Johnson & Johnson (COVID-19 Vaccine Janssen), AstraZeneca (Vaxzevria), Pfizer/BioNTech (Comirnaty), Sputnik (Gam-COVID-Vac), Sinovac (COVID-19 Vaccine (Vero Cell) Inactivated) or Novavax (NVX-CoV2373).

In some embodiments, the immunogenic composition is capable of eliciting a broadly neutralizing antibody response against naturally occurring variants of SARS-COV-2, including the Wuhan index strain as well as variants observed in South Africa, Japan, Brazil, the UK, India and California. In some embodiments, the immunogenic composition is capable of eliciting a neutralizing antibody response against SARS-COV-1. In particular embodiments, the immunogenic composition is capable of eliciting a neutralizing antibody response to a β-coronavirus expressing a spike protein which binds to angiotensin-converting enzyme 2 (ACE2). In particular embodiments, the spike protein is at least 75% (e.g., at least 80%, 90%, 95% or 99%) identical to SEQ ID NO: 1. In particular embodiments, the optimized nucleotide sequence encodes an amino acid sequence comprising SEQ ID NO: 167, optionally wherein the optimized nucleotide sequence has the nucleic acid sequence of SEQ ID NO: 173. In a specific embodiment, the mRNA construct is mRNA construct 2. In particular embodiments, said mRNA construct is encapsulated in a lipid nanoparticle which has a lipid component consisting of cKK-E10, DOPE, cholesterol and DMG-PEG2K, e.g., at the molar ratios 40:30:28.5:1.5. In some embodiments, the immunogenic composition comprises between 7 μg and 135 μg of the mRNA construct, e.g., 7.5 μg, 15 μg, 45 μg or 135 μg.

### Further Exemplary Embodiments of the Invention

In one aspect, the invention provides a nucleic acid comprising an optimized nucleotide sequence encoding a SARS-COV-2 antigen, wherein the optimized nucleotide sequence consists of codons associated with a usage frequency which is greater than or equal to 10%; wherein the optimized nucleotide sequence:
(i) does not contain a termination signal having one of the following nucleotide sequences:
   5′-X₁ATCTX₂TX₃-3′, wherein X₁, X₂ and X₃ are independently selected from A, C, T or G; and 5′-X₁AUCUX₂UX₃-3′, wherein X₁, X₂ and X₃ are independently selected from A, C, U or G;
(ii) does not contain any negative cis-regulatory elements and negative repeat elements; and
(iii) has a codon adaptation index greater than 0.8;
wherein, when divided into non-overlapping 30 nucleotide-long portions, each portion of the optimized nucleotide sequence has a guanine cytosine content range of 30%-70%.

In certain embodiments, the optimized nucleotide sequence does not contain a termination signal having one of the following sequences: TATCTGTT; TTTTTT; AAGCTT; GAAGAGC; TCTAGA; UAUCUGUU; UUUUUU; AAGCUU; GAAGAGC; UCUAGA. In certain embodiments the nucleic acid is mRNA or DNA.

In the following, modified SARS-COV-2 spike proteins or antigenic fragments thereof are described by reference to particular optimized nucleic acid sequences. It should be understood that, although these modified SARS-COV-2 spike protein or an antigenic fragment may have particular utility in the context of the disclosed nucleic acid-based vaccines of the invention, they may also have utility in protein-based vaccines. Moreover, the optimized nucleic acid sequences may also be useful in the efficient production of such protein-based vaccines.

In certain aspects, the nucleic acid of the invention is an optimized nucleotide sequence encoding the SARS-COV-2 spike protein or an antigenic fragment thereof. In certain embodiments, the optimized nucleotide sequence encodes the full-length SARS-COV-2 spike protein. In specific embodiments, the optimized nucleotide sequence encodes an amino acid sequence comprising SEQ ID NO:1. In other embodiments, the nucleic acid of the invention is an optimized nucleotide sequence encoding the ectodomain of the SARS-COV-2 spike protein or an antigenic fragment thereof. In specific embodiments, the optimized nucleotide sequences encode an amino acid sequence comprising SEQ ID NO:2. In certain embodiments, the antigenic fragment comprises the receptor-binding domain (RBD) of the SARS-COV-2 spike protein. In specific embodiments, the optimized nucleotide sequencing encodes an amino acid sequence comprising SEQ ID NO:6.

In certain embodiments, the antigenic fragment further comprises a signal sequence. In certain embodiments, the signal sequence is SEQ ID NO: 7. In other embodiments, the optimized nucleotide sequence of the invention encodes an amino acid sequence comprising SEQ ID NO:8. In certain embodiments, the signal sequence is SEQ ID NO: 142. In other embodiments, the optimized nucleotide sequence of the invention encodes an amino acid sequence comprising SEQ ID NO: 143. In further aspects of the invention the antigenic fragment can additional comprises an Fc region. In specific embodiments, the Fc region has the amino acid sequence of SEQ ID NO: 18. In certain embodiments, the antigenic fragment further comprises a signal sequence and an Fc region.

In certain embodiments, the antigenic fragment consists of the RBD of the SARS-COV-2 spike protein operably linked to a signal sequence and an Fc region. In particular embodiments, the optimized nucleotide sequences encode an amino acid sequence comprising SEQ ID NO:20.

In other embodiments, the SARS-COV-2 spike protein, the ectodomain of the SARS-CoV-2 spike protein or the antigenic fragment thereof has been modified to form a stable prefusion conformation. In certain embodiments, the SARS-COV-2 spike protein, the ectodomain of the SARS-COV-2 spike protein or the antigenic fragment has been modified relative to naturally occurring SARS-COV-2 spike protein to remove the furin cleavage site required for activation. In specific embodiments, the optimized nucleotide sequence encodes a SARS-COV-2 S protein which has been modified relative to naturally occurring SARS-COV-2 spike protein to remove the furin cleavage site required for activation. In further specific embodiments, the optimized nucleotide sequences encode an amino acid sequence comprising SEQ ID NO:9.

In certain embodiments, the SARS-COV-2 spike protein, the ectodomain of the SARS-CoV-2 spike protein or the antigenic fragment thereof has been modified relative to naturally occurring SARS-COV-2 spike protein to mutate residue 985 to proline and/or mutate residues 986 and 987 to proline. In specific embodiments, the optimized nucleotide sequence encodes a SARS-CoV-2 S protein which has been modified relative to naturally occurring SARS-COV-2 spike protein to mutate residues 986 and 987 to proline. In further specific embodiments, the optimized nucleotide sequence encodes an amino acid sequence comprising SEQ ID NO: 10. In further specific embodiments, the optimized nucleotide sequence encodes an amino acid sequence comprising SEQ ID NO: 118.

In certain embodiments, the optimized nucleotide sequence encodes a SARS-COV-2 S protein which has been modified relative to naturally occurring SARS-COV-2 spike protein to mutate residues 985, 986 and 987 to proline. In specific embodiments, the optimized nucleotide sequences encode an amino acid sequence comprising SEQ ID NO: 92.

In certain embodiments, the SARS-COV-2 spike protein, the ectodomain of the SARS-CoV-2 spike protein or the antigenic fragment thereof has been modified relative to naturally occurring SARS-COV-2 spike protein to remove the furin cleavage site and to mutate (a) residues 985 to proline; and/or (b) residues 986 and 987 to proline. In specific embodiments, the SARS-CoV-2 spike protein, the ectodomain of the SARS-COV-2 spike protein or the antigenic fragment thereof has been modified relative to naturally occurring SARS-COV-2 spike protein to remove the furin cleavage site and to mutate residues 986 and 987 to proline. In certain embodiments, the optimized nucleotide sequence encodes a SARS-COV-2 S protein. In specific embodiments, the optimized nucleotide sequence encodes an amino acid sequence comprising SEQ ID NO:11. For example, the optimized nucleotide sequence has the nucleic acid sequence of SEQ ID NO: 44 or SEQ ID NO: 148. In further specific embodiments, the optimized nucleotide sequence encodes an amino acid sequence comprising SEQ ID NO:120. For example, the optimized nucleotide sequence encodes the ectodomain of the SARS-COV-2 S protein. In specific embodiments, the optimized nucleotide sequences encode an amino acid sequence comprising SEQ ID NO:12.

In certain embodiments, the SARS-COV-2 spike protein, the ectodomain of the SARS-CoV-2 spike protein or the antigenic fragment thereof has been modified relative to naturally occurring SARS-COV-2 spike protein to remove the furin cleavage site and to mutate residues 985, 986 and 987 to proline. In specific embodiments, the optimized nucleotide sequence encodes a SARS-COV-2 S protein. In further specific embodiments the optimized nucleotide sequences encodes an amino acid sequence comprising SEQ ID NO:94.

In certain embodiments, the SARS-COV-2 spike protein, the ectodomain thereof or the antigenic fragment thereof has been modified relative to mutate residues 986 and 987 to proline and to contain the D614G mutation. In specific embodiments, the optimized nucleotide sequences encodes an amino acid sequence comprising SEQ ID NO: 118.

In certain embodiments, the SARS-COV-2 spike protein, the ectodomain thereof or the antigenic fragment thereof has been modified relative to remove the furin cleavage site, to mutate residues 986 and 987 to proline and to contain the D614G mutation. In specific embodiments, the optimized nucleotide sequences encodes an amino acid sequence comprising SEQ ID NO: 120.

In certain embodiments, the SARS-COV-2 spike protein, the ectodomain thereof or the antigenic fragment thereof has been modified relative to naturally occurring SARS-COV-2 spike protein to mutate residues by 817, 892, 899, 942, 986 and 987 to proline. In specific embodiments, the optimized nucleotide sequences encodes an amino acid sequence comprising SEQ ID NO: 129.

In certain embodiments, the SARS-COV-2 spike protein, the ectodomain thereof or the antigenic fragment thereof has been modified relative to naturally occurring SARS-COV-2 spike protein to remove the furin cleavage site and to mutate residues by 817, 892, 899, 942, 986 and 987 to proline. In specific embodiments, the optimized nucleotide sequences encodes an amino acid sequence comprising SEQ ID NO: 131.

In certain embodiments, the SARS-COV-2 spike protein, the ectodomain thereof or the antigenic fragment thereof has been modified relative to naturally occurring SARS-COV-2 spike protein to mutate residues 817, 892, 899, 942, 986 and 987 to proline and which contains the D614G mutation. In specific embodiments, the optimized nucleotide sequences encodes an amino acid sequence comprising SEQ ID NO: 133.

In certain embodiments, the SARS-COV-2 spike protein, the ectodomain thereof or the antigenic fragment thereof has been modified relative to naturally occurring SARS-COV-2 spike protein to remove the furin cleavage site and to mutate residues 817, 892, 899, 942, 986 and 987 to proline and which contains the D614G mutation. In specific embodiments, the optimized nucleotide sequences encodes an amino acid sequence comprising SEQ ID NO: 135.

In certain embodiments, the SARS-COV-2 spike protein, the ectodomain thereof or the antigenic fragment thereof has been modified relative to naturally occurring SARS-COV-2 spike protein to remove the furin cleavage site, to mutate residues 986 and 987 to proline and which contains an extended N-terminal signal peptide. In specific embodiments, the optimized nucleotide sequences encodes an amino acid sequence comprising SEQ ID NO: 123. In certain embodiments, the SARS-COV-2 spike protein, the ectodomain thereof or the antigenic fragment thereof has been modified relative to naturally occurring SARS-COV-2 spike protein to remove the furin cleavage site, to mutate residues 817, 892, 899, 942, 986 and 987 to proline and which contains an extended N-terminal signal peptide. In specific embodiments, the optimized nucleotide sequences encodes an amino acid sequence comprising SEQ ID NO: 137.

In certain embodiments, the SARS-COV-2 spike protein, the ectodomain thereof or the antigenic fragment thereof c been modified relative to naturally occurring SARS-COV-2 spike protein to mutate the ER retrieval signal. In certain embodiments, the wherein the SARS-COV-2 spike protein, the ectodomain thereof or the antigenic fragment thereof has been modified relative to naturally occurring SARS-COV-2 spike protein to remove the furin cleavage site, to mutate residues 986 and 987 to proline and to remove the ER retrieval signal. In specific embodiments, the optimized nucleotide sequences encodes an amino acid sequence comprising SEQ ID NO: 125.

In certain embodiments, the SARS-COV-2 spike protein, the ectodomain thereof or the antigenic fragment thereof has been modified relative to naturally occurring SARS-COV-2 spike protein to remove the furin cleavage site, to mutate residues 986 and 987 to proline, to remove the ER retrieval signal and which contains an extended N-terminal signal peptide. In specific embodiments, the optimized nucleotide sequences encodes an amino acid sequence comprising SEQ ID NO: 127.

In certain embodiments, the SARS-COV-2 spike protein, the ectodomain thereof or the antigenic fragment thereof has been modified relative to naturally occurring SARS-COV-2 spike protein to remove the furin cleavage site, to mutate residues 817, 892, 899, 942, 986 and 987 to proline and to remove the ER retrieval signal. In specific embodiments, the optimized nucleotide sequences encodes an amino acid sequence comprising SEQ ID NO: 139.

In certain embodiments, the SARS-COV-2 spike protein, the ectodomain thereof or the antigenic fragment thereof has been modified relative to naturally occurring SARS-COV-2 spike protein to remove the furin cleavage site, to mutate residues 817, 892, 899, 942, 986 and 987 to proline, to remove the ER retrieval signal and which contains an extended N-terminal signal peptide. In specific embodiments, the optimized nucleotide sequences encodes an amino acid sequence comprising SEQ ID NO: 141.

In certain embodiments, an antigenic fragment comprises or consists of the S1, S2 or S2′ subunit of the SARS-COV-2 spike protein. In certain embodiments, the optimized nucleotide sequences encode an amino acid sequence comprising SEQ ID NO: 3, SEQ ID NO: 4 or SEQ ID NO: 5.

In certain embodiments, an optimized nucleotide sequence encodes a fusion peptide comprising one or more antigenic fragments of the SARS-COV-2 S protein. In specific embodiments, the one or more antigenic fragments of the SARS-COV-2 S protein has/have the amino acid sequence of SEQ ID NO: 21, the amino acid sequence SEQ ID NO: 22, the amino acid sequence SEQ ID NO: 23 and/or the amino acid sequence SEQ ID NO: 24.

In certain embodiments, the one or more antigenic fragments are linked by a linker sequence, e.g., GGGGS. In specific embodiments, the optimized nucleotide sequence encodes a fusion peptide comprising SEQ ID NO: 25 or SEQ ID NO: 27. In certain embodiments the fusion peptide comprises an N terminal signal sequence, for example the optimized nucleotide sequence encodes a fusion peptide comprising SEQ ID NO: 51 or SEQ ID NO: 53. In certain embodiments the fusion peptide comprises a C-terminal Fc domain. In other embodiments, the fusion peptide comprises an N terminal signal sequence and a C-terminal Fc domain. In specific embodiments, the optimized nucleotide sequence encodes a fusion peptide comprising SEQ ID NO: 55 or SEQ ID NO: 57.

In other aspects, the nucleic acid of the invention as disclosed above is for use in therapy. For example, the nucleic acid of the invention as disclosed above may be for use in the manufacture of a medicament for the prophylaxis of an infection with SARS-COV-2. In other aspects an immunogenic composition comprising the nucleic acid of the invention for use in prophylaxis of an infection with SARS-COV-2 is provided. The invention also provides methods of treating or preventing a SARS-COV-2 infection, said method comprising administering to a subject an effective amount of an immunogenic composition comprising the nucleic acid of the invention.

In other aspects, an immunogenic composition according to the invention comprises at least two nucleic acids, for use in prophylaxis of an infection with SARS-COV-2 is provided wherein a first nucleic acid comprises an optimized nucleotide sequence which encodes an amino acid sequence comprising a sequence selected from SEQ ID NO: 1, 2, 3, 4, 5, 8, 9, 10, 11, 12, 14, 15, 16, 17, 19, 20, 35, 66, 68, 70, 72, 74, 76, 78, 80, 82, 84. 86, 88, 90, 92, 94, 96, 98, 104, 106, 108, 110, 118, 120, 123, 125, 127, 129, 131, 133, 135, 137, 139 or 141, and wherein one or more further nucleic acid(s) comprise(s) an optimized nucleotide sequence which encodes an amino acid sequence comprising a sequence selected from SEQ ID NO: 151, 153, 155, 157, 159, 161, 163, 165, 167, 169 or 171.

In other aspects, an immunogenic composition according to the invention comprises at least two nucleic acids, for use in prophylaxis of an infection with SARS-COV-2 is provided, wherein a first nucleic acid comprises an optimized nucleotide sequence which encodes an amino acid sequence comprising SEQ ID NO:11, optionally wherein the optimized nucleotide sequence has the nucleic acid sequence of SEQ ID NO: 44, and wherein one or more further nucleic acid(s) is (are) selected from:
(a) a nucleic acid comprising an optimized nucleotide sequence which encodes an amino acid sequence comprising a sequence selected from SEQ ID NO: 157, optionally wherein the optimized nucleotide sequence has the nucleic acid sequence of SEQ ID NO 156, and
(b) nucleic acid comprises an optimized nucleotide sequence that encodes an amino acid sequence comprising a sequence selected from SEQ ID NO: 163, optionally wherein the optimized nucleotide sequence has the nucleic acid sequence of SEQ ID NO: 162; and
(c) nucleic acid comprises an optimized nucleotide sequence that encodes an amino acid sequence comprising a sequence selected from SEQ ID NO: 167, optionally wherein the optimized nucleotide sequence has the nucleic acid sequence of SEQ ID NO: 166; and
(d) nucleic acid comprises an optimized nucleotide sequence that encodes an amino acid sequence comprising a sequence selected from SEQ ID NO: 171, optionally wherein the optimized nucleotide sequence has the nucleic acid sequence of SEQ ID NO: 170.

Certain aspects, the invention provides a pharmaceutical composition comprising i) a nucleic acid of the invention and ii) a lipid nanoparticle. In certain embodiments, the nucleic acid is encapsulated in the lipid nanoparticle. The lipid nanoparticle can comprise one or more of a cationic lipid, a non-cationic lipid, a cholesterol-based lipid, a PEG-modified lipid, or a combination thereof. In certain embodiments, the lipid nanoparticle comprises a cationic lipid, a non-cationic lipid, a cholesterol-based lipid, and a PEG-modified lipid.

In certain embodiments, the lipid nanoparticle comprises a cationic lipid, a non-cationic lipid, and a PEG-modified lipid. In certain embodiments, the lipid nanoparticle comprises:
(a) a cationic lipid selected from DOTAP (1,2-dioleyl-3-trimethylammonium propane), DODAP (1,2-dioleyl-3-dimethylammonium propane), DOTMA (N-[1-(2,3-dioleyloxy) propyl]-N,N,N-trimethylammonium chloride), DLinKC2DMA, DLin-KC2-DM, C12-200, cKK-E12, cKK-E10, HGT5000, HGT5001, HGT4003, ICE, HGT4001, HGT4002, TL1-01D-DMA, TL1-04D-DMA, TL1-08D-DMA, TL1-10D-DMA, OF-Deg-Lin and OF-02;
(b) a non-cationic lipid selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DEPE 1,2-dierucoyl-sn-glycero-3-phosphoethanolamine, DOPC (1,2-dioleyl-sn-glycero-3-phosphotidylcholine), DPPE (1,2-dipalmitoyl-sn-glycero-3-phosphoethanolamine), DMPE (1,2-dimyristoyl-sn-glycero-3-phosphoethanolamine), and DOPG (1,2-dioleoyl-sn-glycero-3-phospho-(l′-rac-glycerol));
(c) a cholesterol-based lipid selected from DC-Choi (N,N-dimethyl-N-ethylcarboxamidocholesterol), 1,4-bis(3-N-oleylamino-propyl) piperazine, or imidazole cholesterol ester (ICE); and/or
(d) a PEG-modified lipid selected from PEGylated cholesterol and DMG-PEG-2K.

In certain embodiments of the pharmaceutical composition the
a. the cationic lipid is selected from cKK-E12, cKK-E10, OF-Deg-Lin and OF-02;
b. the non-cationic lipid is selected from DOPE and DEPE;
c. the cholesterol-based lipid is cholesterol; and
d. the PEG-modified lipid is DMG-PEG-2K.

In certain embodiments, the cationic lipid constitutes about 30-60% of the lipid nanoparticle by molar ratio, e.g., about 35-40%. In certain embodiments, the ratio of cationic lipid to non-cationic lipid to cholesterol-based lipid to PEG-modified lipid is approximately 30-60:25-35:20-30:1-15 by molar ratio or wherein the ratio of cationic lipid to non-cationic lipid to PEG-modified lipid is approximately 55-65:30-40:1-15 by molar ratio.

In certain embodiments, the lipid nanoparticle includes a combination of a cationic lipid, a non-cationic lipid, a PEG-modified lipid and optionally cholesterol selected from cKK-E12, DOPE, cholesterol and DMG-PEG2K; cKK-E10, DOPE, cholesterol and DMG-PEG2K; OF-Deg-Lin, DOPE, cholesterol and DMG-PEG2K; OF-02, DOPE, cholesterol and DMG-PEG2K; TL1-01D-DMA, DOPE, cholesterol and DMG-PEG2K; TL1-04D-DMA, DOPE, cholesterol and DMG-PEG2K; TL1-08D-DMA, DOPE, cholesterol and DMG-PEG2K; TL1-10D-DMA, DOPE, cholesterol and DMG-PEG2K; ICE, DOPE and DMG-PEG2K; HGT4001, DOPE and DMG-PEG2K; or HGT4002, DOPE and DMG-PEG2K.

In certain embodiments, the lipid nanoparticle has an average size of less than 150 nm, e.g., less than 100 nm. In specific embodiments, the lipid nanoparticle has an average size of about 50-70 nm, e.g., about 55-65 nm.

In certain embodiments, the lipid nanoparticles are suspended in 10% trehalose in water for injection. In certain embodiments, the nucleic acid is mRNA at a concentration of between about 0.5 mg/mL to about 1.0 mg/mL.

In certain aspects, the invention provides a pharmaceutical composition comprising i) an optimized nucleic acid of invention (e.g., an mRNA) and ii) a lipid nanoparticle. Such pharmaceutical compositions are for use in treating or preventing an infection with SARS-COV-2. In certain embodiments, the pharmaceutical composition is administered parenterally. In certain embodiments, the pharmaceutical composition is administered intravenously, intradermally, subcutaneously, or intramuscularly. In specific embodiments the pharmaceutical composition is administered intravenously or intramuscularly.

In certain embodiments, the pharmaceutical composition is administered at least once. In specific embodiments, the pharmaceutical composition is administered at least twice. In more specific embodiments, the period between administrations is at least 2 weeks, e.g. 1 month. In some embodiments, the period between administrations is about 3 weeks.

In certain aspects, the invention provides a SARS-COV-2 antigen. For example, the SARS-COV-2 antigen can be any of the SARS-COV-2 spike proteins, antigenic fragments or fusion peptides of antigenic fragments which are described above or in more detail below in reference to particular optimized nucleic acid sequences. In some embodiments, the SARS-COV-2 antigen is a polypeptide comprising or consisting of the amino acid sequence of SEQ ID NO: 1. In some embodiments, the SARS-COV-2 antigen is a polypeptide comprising or consisting of the amino acid sequence of SEQ ID NO: 10 . . . . In some embodiments, the SARS-COV-2 antigen is a polypeptide comprising or consisting of the amino acid sequence of SEQ ID NO: 9. In some embodiments, the SARS-COV-2 antigen is a polypeptide comprising or consisting of the amino acid sequence of SEQ ID NO: 11. In some embodiments, the SARS-COV-2 antigen is a polypeptide comprising or consisting of the amino acid sequence of SEQ ID NO:2. In some embodiments, the SARS-COV-2 antigen is a polypeptide comprising or consisting of the amino acid sequence of SEQ ID NO: 12. In some embodiments, the SARS-COV-2 antigen is a polypeptide comprising or consisting of the amino acid sequence of SEQ ID NO:3. In some embodiments, the SARS-COV-2 antigen is a polypeptide comprising or consisting of the amino acid sequence of SEQ ID NO:8. In some embodiments, the SARS-COV-2 antigen is a polypeptide comprising or consisting of the amino acid sequence of SEQ ID NO:20. In some embodiments, the SARS-COV-2 antigen is a polypeptide comprising or consisting of the amino acid sequence of SEQ ID NO: 17. In some embodiments, the SARS-COV-2 antigen is a polypeptide comprising or consisting of the amino acid sequence of SEQ ID NO:14. In some embodiments, the SARS-COV-2 antigen is a polypeptide comprising or consisting of the amino acid sequence of SEQ ID NO:16. In some embodiments, the SARS-COV-2 antigen is a polypeptide comprising or consisting of the amino acid sequence of SEQ ID NO:66. In some embodiments, the SARS-COV-2 antigen is a polypeptide comprising or consisting of the amino acid sequence of SEQ ID NO:15. In some embodiments, the SARS-COV-2 antigen is a polypeptide comprising or consisting of the amino acid sequence of SEQ ID NO:82. In some embodiments, the SARS-COV-2 antigen is a polypeptide comprising or consisting of the amino acid sequence of SEQ ID NO:84. In some embodiments, the SARS-COV-2 antigen is a polypeptide comprising or consisting of the amino acid sequence of SEQ ID NO:74. In some embodiments, the SARS-COV-2 antigen is a polypeptide comprising or consisting of the amino acid sequence of SEQ ID NO:76. In some embodiments, the SARS-COV-2 antigen is a polypeptide comprising or consisting of the amino acid sequence of SEQ ID NO:78. In some embodiments, the SARS-COV-2 antigen is a polypeptide comprising or consisting of the amino acid sequence of SEQ ID NO:80. In some embodiments, the SARS-COV-2 antigen is a polypeptide comprising or consisting of the amino acid sequence of SEQ ID NO:68. In some embodiments, the SARS-COV-2 antigen is a polypeptide comprising or consisting of the amino acid sequence of SEQ ID NO:70. In some embodiments, the SARS-COV-2 antigen is a polypeptide comprising or consisting of the amino acid sequence of SEQ ID NO:96. In some embodiments, the SARS-COV-2 antigen is a polypeptide comprising or consisting of the amino acid sequence of SEQ ID NO:86. In some embodiments, the SARS-COV-2 antigen is a polypeptide comprising or consisting of the amino acid sequence of SEQ ID NO:88. In some embodiments, the SARS-COV-2 antigen is a polypeptide comprising or consisting of the amino acid sequence of SEQ ID NO:90. In some embodiments, the SARS-COV-2 antigen is a polypeptide comprising or consisting of the amino acid sequence of SEQ ID NO:92. In some embodiments, the SARS-COV-2 antigen is a polypeptide comprising or consisting of the amino acid sequence of SEQ ID NO:94. In some embodiments, the SARS-COV-2 antigen is a polypeptide comprising or consisting of the amino acid sequence of SEQ ID NO: 118. In some embodiments, the SARS-COV-2 antigen is a polypeptide comprising or consisting of the amino acid sequence of SEQ ID NO: 120.

In further aspects, the invention provides a peptide fusion construct comprising one or more antigenic regions of the SARS-COV-2 S protein, where the one or more antigenic regions comprises or consists of the following components: FP, D1, D2 and/or B1, wherein FP comprises residues 815-833 of the SARS-COV-2 S protein, wherein DI comprises residues 820-846 of the SARS-COV-2 S protein, wherein D2 comprises residues 1078-1111 of the SARS-COV-2 S protein, and wherein B1 comprises residues 798-829 of the SARS-COV-2 S protein. The peptide fusion construct may have the following structure: D1-linker-FP-linker-D2-linker-D1. DI may have the sequence of SEQ ID NO: 22. FP may have the sequence of SEQ ID NO: 21. The linker comprises or consists of the amino acid sequence GGGGS. For example, the peptide fusion construct may comprise or consist of the sequence of SEQ ID NO: 25 or 51, 55. Alternatively, the peptide fusion construct may have the following structure: FP-linker-FP-linker-FP, D1-linker-D1-linker-D1, or FP/D1-linker-FP/D1-linker-FP/D1. The FP/D1 portion may have the sequence of SEQ ID NO: 99. The linker may comprise or consist of the amino acid sequence GGGGS. For example, the peptide fusion construct may comprise or consist of the sequence of SEQ ID NO: 27 or 53, 57

The invention also provides a pharmaceutical composition comprising the SARS-COV-2 antigen or the peptide fusion construct of the invention. In some embodiments, the pharmaceutical composition further comprising an adjuvant. In certain embodiments, the adjuvant is selected from alum, CpG, PolyI:C, MF59, AS01, AS02, AS03, AS04, AF03, flagellin, ISCOMs and ISCOMMATRIX. In some aspects, the pharmaceutical composition is for use in treating or preventing an infection with SARS-COV-2. In some embodiments, the pharmaceutical composition is administered parenterally. In some embodiments, the pharmaceutical composition is administered intradermally, subcutaneously, or intramuscularly. In some embodiments, the pharmaceutical composition is administered intramuscularly. In some embodiments, the pharmaceutical composition is administered at least once. In some embodiments, the pharmaceutical composition is administered at least twice. In some embodiments, the period between administrations is at least 2 weeks, e.g. 1 month. In some embodiments, the period between administrations is about 3 weeks.

In a particular embodiment, the invention provides an mRNA construct consisting of the following structural elements:
(i) a 5′ cap with the following structure:

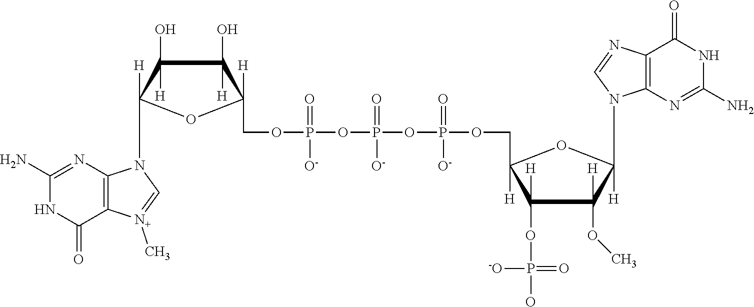

(ii) a 5′ untranslated region (5′ UTR) having the nucleic acid sequence of SEQ ID NO: 144;
(iii) a protein coding region having the nucleic acid sequence of SEQ ID NO: 148;
(iv) a 3′ untranslated region (3′ UTR) having the nucleic acid sequence of SEQ ID NO: 145; and
(v) a polyA tail.

In a specific embodiment, the invention provides a lipid nanoparticle encapsulating said mRNA construct. The lipid nanoparticle may comprise a cationic lipid (e.g., cKK-E12, cKK-E10, OF-Deg-Lin or OF-02), a non-cationic lipid (e.g., DOPE or DEPE), a cholesterol-based lipid (e.g., cholesterol) and a PEG-modified lipid (e.g., DMG-PEG-2K). In a particular embodiment, the mRNA construct or the lipid nanoparticle encapsulating it are provided as an immunogenic composition. In some embodiments, the immunogenic composition comprises between 10 μg and 200 μg of the mRNA construct. In particular embodiments, the immunogenic composition comprises between 15 μg and 135 μg (e.g., between 15 μg and 45 μg) of the mRNA construct. In some embodiments, the immunogenic composition may comprise at least 20 μg, at least 25 μg, at least 30 μg, at least 35 μg, at least 40 μg, or at least 45 μg of the mRNA construct. In specific embodiments, the immunogenic composition comprises 15 μg, 45 μg or 135 μg of the mRNA construct. The invention further provides a method of treating or preventing a SARS-COV-2 infection, wherein said method comprises administering to a subject an effective amount of the immunogenic composition. In some embodiments, the immunogenic is administered to the subject at least twice. In some embodiments, the period between administrations is at least 2 weeks. In some embodiments, the period between administrations is about 3 weeks.

In certain embodiments, the invention is further described by the following numbered embodiments:
1. A nucleic acid comprising an optimized nucleotide sequence encoding a SARS-COV-2 antigen, wherein the optimized nucleotide sequence consists of codons associated with a usage frequency which is greater than or equal to 10%; wherein the optimized nucleotide sequence:
(i) does not contain a termination signal having one of the following nucleotide sequences:
5′-X₁ATCTX₂TX₃-3′, wherein X₁, X₂ and X₃ are independently selected from A, C, T or G; and 5′-X₁AUCUX₂UX₃-3′, wherein X₁, X₂ and X₃ are independently selected from A, C, U or G;
(ii) does not contain any negative cis-regulatory elements and negative repeat elements; and
(iii) has a codon adaptation index greater than 0.8;
wherein, when divided into non-overlapping 30 nucleotide-long portions, each portion of the optimized nucleotide sequence has a guanine cytosine content range of 30%-70%.
2. The nucleic acid of embodiment 1, wherein the optimized nucleotide sequence does not contain a termination signal having one of the following sequences: TATCTGTT; TTTTTT; AAGCTT; GAAGAGC; TCTAGA; UAUCUGUU; UUUUUU; AAGCUU; GAAGAGC; UCUAGA.
3. The nucleic acid of embodiment 1 or 2, wherein the nucleic acid is mRNA.
4. The nucleic acid of embodiment 1 or 2, wherein the nucleic acid is DNA.
5. The nucleic acid of any one of the preceding embodiments, wherein the optimized nucleotide sequence encodes the SARS-COV-2 spike protein or an antigenic fragment thereof.
6. The nucleic acid of embodiment 5, wherein the optimized nucleotide sequence encodes the full-length SARS-COV-2 spike protein.
7. The nucleic acid of embodiment 5 or embodiment 6, wherein the optimized nucleotide sequences encodes an amino acid sequence comprising SEQ ID NO:1.
8. The nucleic acid of embodiment 5, wherein the optimized nucleotide sequence encodes the ectodomain of the SARS-COV-2 spike protein or an antigenic fragment thereof.
9 The nucleic acid of embodiment 8, wherein the optimized nucleotide sequences encodes an amino acid sequence comprising SEQ ID NO:2.
10. The nucleic acid of embodiment 5, wherein the antigenic fragment comprises the receptor-binding domain (RBD) of the SARS-COV-2 spike protein.
11. The nucleic acid of embodiment 10, wherein the optimized nucleotide sequences encodes an amino acid sequence comprising SEQ ID NO:6.
12. The nucleic acid of embodiment 10 or 11, wherein the antigenic fragment further comprises a signal sequence.
13. The nucleic acid of embodiment 12, wherein the signal sequence is SEQ ID NO: 7.
14. The nucleic acid of embodiment 12 or embodiment 13, wherein the optimized nucleotide sequences encodes an amino acid sequence comprising SEQ ID NO:8.
15. The nucleic acid of embodiment 12, wherein the signal sequence is SEQ ID NO: 142.
16. The nucleic acid of embodiment 12 or embodiment 13, wherein the optimized nucleotide sequences encodes an amino acid sequence comprising SEQ ID NO:143.
17. The nucleic acid of embodiments 10-16, wherein the antigenic fragment further comprises an Fc region.
18. The nucleic acid of embodiment 17, wherein the Fc region is SEQ ID NO: 18.
19. The nucleic acid of embodiments 10-18, wherein the antigenic fragment further comprises a signal sequence and an Fc region.
20. The nucleic acid of embodiments 10-18, wherein the antigenic fragment consists of the RBD of the SARS-COV-2 spike protein operably linked to a signal sequence and an Fc region.
21. The nucleic acid of embodiment 20, wherein the optimized nucleotide sequences encodes an amino acid sequence comprising SEQ ID NO:20.
22. The nucleic acid of any one of embodiment 5, embodiment 6 or embodiment 8, wherein the SARS-COV-2 spike protein, the ectodomain thereof or the antigenic fragment thereof has been modified relative to naturally occurring SARS-COV-2 spike protein to assume a stable prefusion conformation.
23. The nucleic acid of embodiment 22, wherein the SARS-COV-2 spike protein, the ectodomain or the antigenic fragment has been modified relative to naturally occurring SARS-CoV-2 spike protein to remove the furin cleavage site required for activation.
24. The nucleic acid of embodiment 23, wherein the optimized nucleotide sequence encodes a SARS-COV-2 spike protein which has been modified relative to naturally occurring SARS-COV-2 spike protein to remove the furin cleavage site required for activation
25. The nucleic acid of embodiment 23, wherein the optimized nucleotide sequences encodes an amino acid sequence comprising SEQ ID NO:9.
26. The nucleic acid of embodiments 22-25, wherein the SARS-COV-2 spike protein, the ectodomain thereof or the antigenic fragment thereof has been modified relative to naturally occurring SARS-COV-2 spike protein to mutate residue 985 to proline and/or mutate residues 986 and 987 to proline.
27. The nucleic acid of embodiment 26, wherein the optimized nucleotide sequence encodes a SARS-COV-2 spike protein which has been modified relative to naturally occurring SARS-COV-2 spike protein to mutate residues 986 and 987 to proline.
28. The nucleic acid of embodiment 27, wherein the optimized nucleotide sequences encodes an amino acid sequence comprising SEQ ID NO: 10 or SEQ ID NO: 118.
29. The nucleic acid of embodiment 26, wherein the optimized nucleotide sequence encodes a SARS-COV-2 spike protein which has been modified relative to naturally occurring SARS-COV-2 spike protein to mutate residues 985, 986 and 987 to proline.
30. The nucleic acid of embodiment 29, wherein the optimized nucleotide sequences encodes an amino acid sequence comprising SEQ ID NO:92.
31. The nucleic acid of embodiments 22-30, wherein the SARS-COV-2 spike protein, the ectodomain thereof or the antigenic fragment thereof has been modified relative to naturally occurring SARS-COV-2 spike protein to remove the furin cleavage site and to mutate
(a) residues 985 to proline; and/or
(b) residues 986 and 987 to proline.
32. The nucleic acid to embodiment 31, wherein the SARS-COV-2 spike protein, the ectodomain of the SARS-COV-2 spike protein or the antigenic fragment thereof has been modified relative to naturally occurring SARS-COV-2 spike protein to remove the furin cleavage site and to mutate residues 986 and 987 to proline.
33. The nucleic acid of embodiment 32, wherein the optimized nucleotide sequence encodes a SARS-COV-2 spike protein.
34. The nucleic acid of embodiment 33, wherein the optimized nucleotide sequences encodes an amino acid sequence comprising SEQ ID NO:11 or SEQ ID NO: 120, optionally wherein the optimized nucleotide sequence has the nucleic acid sequence of SEQ ID NO: 44 or SEQ ID NO: 148.
35. The nucleic acid of embodiment 32, wherein the optimized nucleotide sequence encodes the ectodomain of the SARS-COV-2 spike protein.
36. The nucleic acid of embodiment 35, wherein the optimized nucleotide sequences encodes an amino acid sequence comprising SEQ ID NO:12.
37. The nucleic acid to embodiment 31, wherein the SARS-COV-2 spike protein, the ectodomain thereof or the antigenic fragment thereof has been modified relative to naturally occurring SARS-COV-2 spike protein to remove the furin cleavage site and to mutate residues 985, 986 and 987 to proline.
38. The nucleic acid of embodiment 37, wherein the optimized nucleotide sequence encodes a SARS-COV-2 spike protein.
39. The nucleic acid of embodiment 38, wherein the optimized nucleotide sequences encodes an amino acid sequence comprising SEQ ID NO:94.
40. The nucleic acid of embodiments 22-39, wherein the SARS-COV-2 spike protein, the ectodomain thereof or the antigenic fragment thereof has been modified relative to mutate residues 986 and 987 to proline and to contain the D614G mutation.
41. The nucleic acid of embodiment 40, wherein the optimized nucleotide sequences encodes an amino acid sequence comprising SEQ ID NO: 118.
42. The nucleic acid of embodiments 22-41, wherein the SARS-COV-2 spike protein, the ectodomain thereof or the antigenic fragment thereof has been modified relative to remove the furin cleavage site, to mutate residues 986 and 987 to proline and to contain the D614G mutation
43. The nucleic acid of embodiment 42, wherein the optimized nucleotide sequences encodes an amino acid sequence comprising SEQ ID NO: 120.
44. The nucleic acid of embodiments 22-43, wherein the SARS-COV-2 spike protein, the ectodomain thereof or the antigenic fragment thereof has been modified relative to naturally occurring SARS-COV-2 spike protein to mutate residues by 817, 892, 899, 942, 986 and 987 to proline.
45. The nucleic acid of embodiment 44, wherein the optimized nucleotide sequences encodes an amino acid sequence comprising SEQ ID NO: 129.
46. The nucleic acid of embodiments 22-45, wherein the SARS-COV-2 spike protein, the ectodomain thereof or the antigenic fragment thereof has been modified relative to naturally occurring SARS-COV-2 spike protein to remove the furin cleavage site and to mutate residues by 817, 892, 899, 942, 986 and 987 to proline.
47. The nucleic acid of embodiment 46, wherein the optimized nucleotide sequences encodes an amino acid sequence comprising SEQ ID NO: 131.
48. The nucleic acid of embodiments 22-47, wherein the SARS-COV-2 spike protein, the ectodomain thereof or the antigenic fragment thereof has been modified relative to naturally occurring SARS-COV-2 spike protein to mutate residues 817, 892, 899, 942, 986 and 987 to proline and which contains the D614G mutation.
49. The nucleic acid of embodiment 48, wherein the optimized nucleotide sequences encodes an amino acid sequence comprising SEQ ID NO: 133.
50. The nucleic acid of embodiments 22-49, wherein the SARS-COV-2 spike protein, the ectodomain thereof or the antigenic fragment thereof has been modified relative to naturally occurring SARS-COV-2 spike protein to remove the furin cleavage site and to mutate residues 817, 892, 899, 942, 986 and 987 to proline and which contains the D614G mutation.
51. The nucleic acid of embodiment 50, wherein the optimized nucleotide sequences encodes an amino acid sequence comprising SEQ ID NO: 135.
52. The nucleic acid of embodiments 22-51, wherein the SARS-COV-2 spike protein, the ectodomain thereof or the antigenic fragment thereof has been modified relative to naturally occurring SARS-COV-2 spike protein to remove the furin cleavage site, to mutate residues 986 and 987 to proline and which contains an extended N-terminal signal peptide.
53. The nucleic acid of embodiment 52, wherein the optimized nucleotide sequences encodes an amino acid sequence comprising SEQ ID NO: 123.
54. The nucleic acid of embodiments 22-53, wherein the SARS-COV-2 spike protein, the ectodomain thereof or the antigenic fragment thereof has been modified relative to naturally occurring SARS-COV-2 spike protein to remove the furin cleavage site, to mutate residues 817, 892, 899, 942, 986 and 987 to proline and which contains an extended N-terminal signal peptide.
55. The nucleic acid of embodiment 54, wherein the optimized nucleotide sequences encodes an amino acid sequence comprising SEQ ID NO: 137.
56. The nucleic acid of embodiments 22-55, wherein the SARS-COV-2 spike protein, the ectodomain thereof or the antigenic fragment thereof has been modified relative to naturally occurring SARS-COV-2 spike protein to mutate the ER retrieval signal.
57. The nucleic acid of embodiment 56, wherein the SARS-COV-2 spike protein, the ectodomain thereof or the antigenic fragment thereof has been modified relative to naturally occurring SARS-COV-2 spike protein to remove the furin cleavage site, to mutate residues 986 and 987 to proline and to remove the ER retrieval signal.
58. The nucleic acid of embodiment 57, wherein the optimized nucleotide sequences encodes an amino acid sequence comprising SEQ ID NO: 125.
59. The nucleic acid of embodiment 56, wherein the SARS-COV-2 spike protein, the ectodomain thereof or the antigenic fragment thereof has been modified relative to naturally occurring SARS-COV-2 spike protein to remove the furin cleavage site, to mutate residues 986 and 987 to proline, to remove the ER retrieval signal and which contains an extended N-terminal signal peptide.
60. The nucleic acid of embodiment 59, wherein the optimized nucleotide sequences encodes an amino acid sequence comprising SEQ ID NO: 127.
61. The nucleic acid of embodiment 56, wherein the SARS-COV-2 spike protein, the ectodomain thereof or the antigenic fragment thereof has been modified relative to naturally occurring SARS-COV-2 spike protein to remove the furin cleavage site, to mutate residues 817, 892, 899, 942, 986 and 987 to proline and to remove the ER retrieval signal.
62. The nucleic acid of embodiment 61, wherein the optimized nucleotide sequences encodes an amino acid sequence comprising SEQ ID NO: 139.
63. The nucleic acid of embodiment 56, wherein the SARS-COV-2 spike protein, the ectodomain thereof or the antigenic fragment thereof has been modified relative to naturally occurring SARS-COV-2 spike protein to remove the furin cleavage site, to mutate residues 817, 892, 899, 942, 986 and 987 to proline, to remove the ER retrieval signal and which contains an extended N-terminal signal peptide.
64. The nucleic acid of embodiment 63, wherein the optimized nucleotide sequences encodes an amino acid sequence comprising SEQ ID NO: 141.
65. The nucleic acid of embodiment 5, wherein the antigenic fragment comprises or consists of the S1, S2 or S2′ subunit of the SARS-COV-2 spike protein.
66. The nucleic acid of embodiment 65, wherein the optimized nucleotide sequences encodes an amino acid sequence comprising SEQ ID NO: 3, SEQ ID NO: 4 or SEQ ID NO: 5.
67. The nucleic acid of embodiments 1-4, wherein the optimized nucleotide sequence encodes a fusion peptide comprising one or more antigenic fragments of the SARS-COV-2 spike protein.
68. The nucleic acid of embodiment 67, wherein the one or more antigenic fragments of the SARS-COV-2 spike protein has/have the amino acid sequence of SEQ ID NO: 21, the amino acid sequence SEQ ID NO: 22, the amino acid sequence SEQ ID NO: 23 and/or the amino acid sequence SEQ ID NO: 24.
69. The nucleic acid of embodiment 67 or 68, wherein the one or more antigenic fragments are linked by a linker sequence, e.g., GGGGS.
70. The nucleic acid of embodiment 69, wherein the optimized nucleotide sequence encodes a fusion peptide comprising SEQ ID NO: 25 or SEQ ID NO: 27.
71. The nucleic acid of embodiment 67-70, wherein the fusion peptide comprises an N terminal signal sequence.
72. The nucleic acid of embodiment 71, wherein the optimized nucleotide sequence encodes a fusion peptide comprising SEQ ID NO: 51 or SEQ ID NO: 53.
73. The nucleic acid of embodiment 67-72, wherein the fusion peptide comprises a C-terminal Fc domain.
74. The nucleic acid of embodiment 67-73 wherein the fusion peptide comprises an N terminal signal sequence and a C-terminal Fc domain.
75. The nucleic acid of embodiment 74, wherein the optimized nucleotide sequence encodes a fusion peptide comprising SEQ ID NO: 55 or SEQ ID NO: 57.
76. The nucleic acid of any one of embodiments 1 to 75 for use in therapy.
77. An immunogenic composition comprising the nucleic acid of any one of embodiments 1-76 for use in prophylaxis of an infection with SARS-COV-2.
78. A method of treating or preventing a SARS-COV-2 infection, said method comprising administering to a subject an effective amount of an immunogenic composition comprising the nucleic acid of any one of embodiments 1-76.
79. A pharmaceutical composition comprising i) the nucleic acid of any one of embodiments 1-76 and ii) a lipid nanoparticle.
80. The pharmaceutical composition of embodiment 79, wherein the nucleic acid is encapsulated in the lipid nanoparticle.
81. The pharmaceutical composition of embodiment 79 or embodiment 80, wherein the lipid nanoparticle comprises one or more of a cationic lipid, a non-cationic lipid, a cholesterol-based lipid, a PEG-modified lipid, or a combination thereof.
82. The pharmaceutical composition of embodiment 81, wherein the lipid nanoparticle comprises a cationic lipid, a non-cationic lipid, a cholesterol-based lipid, and a PEG-modified lipid.
83. The pharmaceutical composition of embodiment 79, wherein the lipid nanoparticle comprises a cationic lipid, a non-cationic lipid, and a PEG-modified lipid.
84. The pharmaceutical composition of any one of embodiments 79-83, wherein the lipid nanoparticle comprises:
a. a cationic lipid selected from DOTAP (1,2-dioleyl-3-trimethylammonium propane), DODAP (1,2-dioleyl-3-dimethylammonium propane), DOTMA (N-[1-(2,3-dioleyloxy) propyl]-N,N,N-trimethylammonium chloride), DLinKC2DMA, DLin-KC2-DM, C12-200, cKK-E12, cKK-E10, HGT5000, HGT5001, HGT4003, ICE, HGT4001, HGT4002, TL1-01D-DMA, TL1-04D-DMA, TL1-08D-DMA, TL1-10D-DMA, OF-Deg-Lin and OF-02;
b. a non-cationic lipid selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DEPE 1,2-dierucoyl-sn-glycero-3-phosphoethanolamine, DOPC (1,2-dioleyl-sn-glycero-3-phosphotidylcholine), DPPE (1,2-dipalmitoyl-sn-glycero-3-phosphoethanolamine), DMPE (1,2-dimyristoyl-sn-glycero-3-phosphoethanolamine), and DOPG (1,2-dioleoyl-sn-glycero-3-phospho-(1′-rac-glycerol));
c. a cholesterol-based lipid selected from DC-Choi (N,N-dimethyl-N-ethylcarboxamidocholesterol), 1,4-bis(3-N-oleylamino-propyl) piperazine, or imidazole cholesterol ester (ICE); and/or
d. a PEG-modified lipid selected from PEGylated cholesterol and DMG-PEG-2K.
85. The pharmaceutical composition of embodiment 82, wherein
a. the cationic lipid is selected from cKK-E12, cKK-E10, OF-Deg-Lin and OF-02;
b. the non-cationic lipid is selected from DOPE and DEPE;
c. the cholesterol-based lipid is cholesterol; and
d. the PEG-modified lipid is DMG-PEG-2K.
86. The pharmaceutical composition of any one of embodiments 79-85, wherein cationic lipid constitutes about 30-60% of the lipid nanoparticle by molar ratio, e.g., about 35-40%.
87. The pharmaceutical composition of any one of embodiments 79-86, wherein the ratio of cationic lipid to non-cationic lipid to cholesterol-based lipid to PEG-modified lipid is approximately 30-60:25-35:20-30:1-15 by molar ratio or wherein the ratio of cationic lipid to non-cationic lipid to PEG-modified lipid is approximately 55-65:30-40:1-15 by molar ratio.
88. The pharmaceutical composition of any one of embodiments 79-87, wherein the lipid nanoparticle includes a combination of a cationic lipid, a non-cationic lipid, a PEG-modified lipid and optionally cholesterol selected from cKK-E12, DOPE, cholesterol and DMG-PEG2K; cKK-E10, DOPE, cholesterol and DMG-PEG2K; OF-Deg-Lin, DOPE, cholesterol and DMG-PEG2K; OF-02, DOPE, cholesterol and DMG-PEG2K; TL1-01D-DMA, DOPE, cholesterol and DMG-PEG2K; TL1-04D-DMA, DOPE, cholesterol and DMG-PEG2K; TL1-08D-DMA, DOPE, cholesterol and DMG-PEG2K; TL1-10D-DMA, DOPE, cholesterol and DMG-PEG2K; ICE, DOPE and DMG-PEG2K; HGT4001, DOPE and DMG-PEG2K; or HGT4002, DOPE and DMG-PEG2K.
89. The pharmaceutical composition of any one of embodiments 79-88, wherein the lipid nanoparticle has an average size of less than 150 nm, e.g., less than 100 nm.
90. The pharmaceutical composition of embodiment 89, wherein the lipid nanoparticle has an average size of about 50-70 nm, e.g., about 55-65 nm.
91. The pharmaceutical composition any one of embodiments 79-90, wherein the lipid nanoparticles are suspended in 10% trehalose in water for injection.
92. The pharmaceutical composition any one of embodiments 79-91, wherein the nucleic acid is mRNA at a concentration of between about 0.5 mg/mL to about 1.0 mg/mL.
93. The pharmaceutical composition of any one of embodiments 79-92 for use in treating or preventing an infection with SARS-COV-2.
94. The pharmaceutical composition for use according to embodiment 79-93, wherein the pharmaceutical composition is administered parenterally.
95. The pharmaceutical composition for use according to embodiment 79-93, wherein the pharmaceutical composition is administered intravenously, intradermally, subcutaneously, or intramuscularly.
96. The pharmaceutical composition for use according to embodiment 95, wherein the pharmaceutical composition is administered intravenously.
97. The pharmaceutical composition for use according to embodiment 95, wherein the pharmaceutical composition is administered intramuscularly.
98. The pharmaceutical composition for use according to any one of embodiments 79-97, wherein the pharmaceutical composition is administered at least once.
99. The pharmaceutical composition for use according to embodiment 98, wherein the pharmaceutical composition is administered at least twice.
100. The pharmaceutical composition for use according to embodiment 99, wherein the period between administrations is at least 2 weeks, e.g. 3 weeks, or 1 month.
101. A SARS-COV-2 antigen, wherein the SARS-COV-2 antigen is a polypeptide comprising or consisting of the amino acid sequence of SEQ ID NO: 1.
102. A SARS-COV-2 antigen, wherein the SARS-COV-2 antigen is a polypeptide comprising or consisting of the amino acid sequence of SEQ ID NO: 10.
103. A SARS-COV-2 antigen, wherein the SARS-COV-2 antigen is a polypeptide comprising or consisting of the amino acid sequence of SEQ ID NO: 9.
104. A SARS-COV-2 antigen, wherein the SARS-COV-2 antigen is a polypeptide comprising or consisting of the amino acid sequence of SEQ ID NO: 11.
105. A SARS-COV-2 antigen, wherein the SARS-COV-2 antigen is a polypeptide comprising or consisting of the amino acid sequence of SEQ ID NO:2.
106. A SARS-COV-2 antigen, wherein the SARS-COV-2 antigen is a polypeptide comprising or consisting of the amino acid sequence of SEQ ID NO:12.
107. A SARS-COV-2 antigen, wherein the SARS-COV-2 antigen is a polypeptide comprising or consisting of the amino acid sequence of SEQ ID NO:3.
108. A SARS-COV-2 antigen, wherein the SARS-COV-2 antigen is a polypeptide comprising or consisting of the amino acid sequence of SEQ ID NO:8.
109. A SARS-COV-2 antigen, wherein the SARS-COV-2 antigen is a polypeptide comprising or consisting of the amino acid sequence of SEQ ID NO:20.
110. A SARS-COV-2 antigen, wherein the SARS-COV-2 antigen is a polypeptide comprising or consisting of the amino acid sequence of SEQ ID NO: 17.
111. A SARS-COV-2 antigen, wherein the SARS-COV-2 antigen is a polypeptide comprising or consisting of the amino acid sequence of SEQ ID NO: 14.
112. A SARS-COV-2 antigen, wherein the SARS-COV-2 antigen is a polypeptide comprising or consisting of the amino acid sequence of SEQ ID NO:16.
113. A SARS-COV-2 antigen, wherein the SARS-COV-2 antigen is a polypeptide comprising or consisting of the amino acid sequence of SEQ ID NO:66.
114. A SARS-COV-2 antigen, wherein the SARS-COV-2 antigen is a polypeptide comprising or consisting of the amino acid sequence of SEQ ID NO:15.
115. A SARS-COV-2 antigen, wherein the SARS-COV-2 antigen is a polypeptide comprising or consisting of the amino acid sequence of SEQ ID NO:82.
116. A SARS-COV-2 antigen, wherein the SARS-COV-2 antigen is a polypeptide comprising or consisting of the amino acid sequence of SEQ ID NO:84.
117. A SARS-COV-2 antigen, wherein the SARS-COV-2 antigen is a polypeptide comprising or consisting of the amino acid sequence of SEQ ID NO:74.
118. A SARS-COV-2 antigen, wherein the SARS-COV-2 antigen is a polypeptide comprising or consisting of the amino acid sequence of SEQ ID NO:76.
119. A SARS-COV-2 antigen, wherein the SARS-COV-2 antigen is a polypeptide comprising or consisting of the amino acid sequence of SEQ ID NO:78.
120. A SARS-COV-2 antigen, wherein the SARS-COV-2 antigen is a polypeptide comprising or consisting of the amino acid sequence of SEQ ID NO:80.
121. A SARS-COV-2 antigen, wherein the SARS-COV-2 antigen is a polypeptide comprising or consisting of the amino acid sequence of SEQ ID NO:68.
122. A SARS-COV-2 antigen, wherein the SARS-COV-2 antigen is a polypeptide comprising or consisting of the amino acid sequence of SEQ ID NO:70.
123. A SARS-COV-2 antigen, wherein the SARS-COV-2 antigen is a polypeptide comprising or consisting of the amino acid sequence of SEQ ID NO:96.
124. A SARS-COV-2 antigen, wherein the SARS-COV-2 antigen is a polypeptide comprising or consisting of the amino acid sequence of SEQ ID NO:86.
125. A SARS-COV-2 antigen, wherein the SARS-COV-2 antigen is a polypeptide comprising or consisting of the amino acid sequence of SEQ ID NO:88.
126. A SARS-COV-2 antigen, wherein the SARS-COV-2 antigen is a polypeptide comprising or consisting of the amino acid sequence of SEQ ID NO:90.
127. A SARS-COV-2 antigen, wherein the SARS-COV-2 antigen is a polypeptide comprising or consisting of the amino acid sequence of SEQ ID NO:92.
128. A SARS-COV-2 antigen, wherein the SARS-COV-2 antigen is a polypeptide comprising or consisting of the amino acid sequence of SEQ ID NO:94.
129. A SARS-COV-2 antigen, wherein the SARS-COV-2 antigen is a polypeptide comprising or consisting of the amino acid sequence of SEQ ID NO: 118.
130. A SARS-COV-2 antigen, wherein the SARS-COV-2 antigen is a polypeptide comprising or consisting of the amino acid sequence of SEQ ID NO: 120.
131. A SARS-COV-2 antigen, wherein the SARS-COV-2 antigen is a polypeptide comprising or consisting of the amino acid sequence of SEQ ID NO: 123.
132. A SARS-COV-2 antigen, wherein the SARS-COV-2 antigen is a polypeptide comprising or consisting of the amino acid sequence of SEQ ID NO: 125.
133. A SARS-COV-2 antigen, wherein the SARS-COV-2 antigen is a polypeptide comprising or consisting of the amino acid sequence of SEQ ID NO: 127.
134. A SARS-COV-2 antigen, wherein the SARS-COV-2 antigen is a polypeptide comprising or consisting of the amino acid sequence of SEQ ID NO: 129.
135. A SARS-COV-2 antigen, wherein the SARS-COV-2 antigen is a polypeptide comprising or consisting of the amino acid sequence of SEQ ID NO: 131.
136. A SARS-COV-2 antigen, wherein the SARS-COV-2 antigen is a polypeptide comprising or consisting of the amino acid sequence of SEQ ID NO: 133.
137. A SARS-COV-2 antigen, wherein the SARS-COV-2 antigen is a polypeptide comprising or consisting of the amino acid sequence of SEQ ID NO: 135.
138. A SARS-COV-2 antigen, wherein the SARS-COV-2 antigen is a polypeptide comprising or consisting of the amino acid sequence of SEQ ID NO: 139.
139. A SARS-COV-2 antigen, wherein the SARS-COV-2 antigen is a polypeptide comprising or consisting of the amino acid sequence of SEQ ID NO: 141.
140. A peptide fusion construct comprising one or more antigenic regions of the SARS-COV-2 S protein, where the one or more antigenic regions comprises or consists of the following components: FP, D1, D2 and/or B1, wherein FP comprises residues 815-833 of the SARS-COV-2 S protein, wherein D1 comprises residues 820-846 of the SARS-COV-2 S protein, wherein D2 comprises residues 1078-1111 of the SARS-COV-2 S protein, and wherein B1 comprises residues 798-829 of the SARS-COV-2 S protein.
141. The peptide fusion construct according to embodiment 140, wherein the peptide fusion construct has the following structure: D1-linker-FP-linker-D2-linker-D1,
142. The peptide fusion construct according to embodiment 141, wherein DI has the sequence of SEQ ID NO: 22.
143. The peptide fusion construct according to embodiment 140 or 141, wherein FP has the sequence of SEQ ID NO: 21
144. The peptide fusion construct according to any one of embodiments 140, 141 and 142, wherein the linker comprises or consists of the amino acid sequence GGGGS.
145. The peptide fusion construct according to any one of embodiments 140-144, comprising or consisting of the sequence of SEQ ID NO: 25 or 51, 55,
146. The peptide fusion construct according to embodiment 140, wherein the peptide fusion construct has the following structure: FP-linker-FP-linker-FP, D1-linker-D1-linker-D1, or FP/D1-linker-FP/D1-linker-FP/D1.
147. The peptide fusion construct according to embodiment 146, wherein the FP/D1 portion has the sequence of SEQ ID NO: 99.
148. The peptide fusion construct according to embodiment 146 or 147, wherein the linker comprises or consists of the amino acid sequence GGGGS.
149. The peptide fusion construct according to any one of embodiments 146-148, comprising or consisting of the sequence of SEQ ID NO: 27 or 53, 57.
150. A pharmaceutical composition comprising the SARS-COV-2 antigen of any one of embodiments 101-131 or the peptide fusion construct of any one of embodiments 146-149.
151. The pharmaceutical composition of embodiment 150, further comprising an adjuvant.
152. The pharmaceutical composition of embodiment 151, wherein the adjuvant is selected from alum, CpG, PolyI: C, MF59, AS01, AS02, AS03, AS04, AF03, flagellin, ISCOMs and ISCOMMATRIX.
153. The pharmaceutical composition of any one of embodiments 150-152 for use in treating or preventing an infection with SARS-COV-2.
154. The pharmaceutical composition for use according to embodiment 153, wherein the pharmaceutical composition is administered parenterally.
155. The pharmaceutical composition for use according to embodiment 154, wherein the pharmaceutical composition is administered intradermally, subcutaneously, or intramuscularly.
156. The pharmaceutical composition for use according to embodiment 155, wherein the pharmaceutical composition is administered intramuscularly.
157. The pharmaceutical composition for use according to any one of embodiments 153-156, wherein the pharmaceutical composition is administered at least once.
158. The pharmaceutical composition for use according to embodiments 153-156, wherein the pharmaceutical composition is administered at least twice.
159. The pharmaceutical composition for use according to embodiments 158, wherein the period between administrations is at least 2 weeks, e.g. 3 weeks, or 1 month.
160. An mRNA construct consisting of the following structural elements:
(i) a 5′ cap with the following structure:

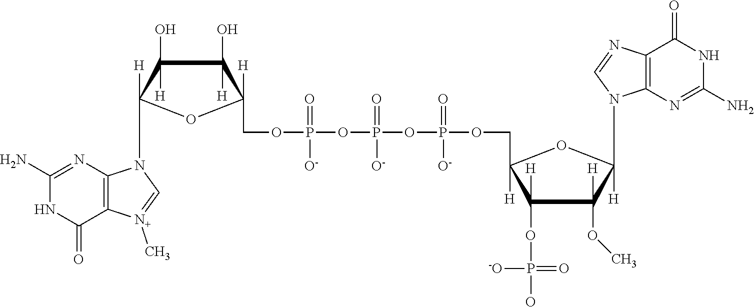

(ii) a 5′ untranslated region (5′ UTR) having the nucleic acid sequence of SEQ ID NO: 144;
(iii) a protein coding region having the nucleic acid sequence of SEQ ID NO: 148;
(iv) a 3′ untranslated region (3′ UTR) having the nucleic acid sequence of SEQ ID NO: 145; and
(v) a poly A tail.
161. A lipid nanoparticle encapsulating the mRNA construct of embodiment 160.
162. The lipid nanoparticle of embodiment 161, wherein the lipid nanoparticle comprises a cationic lipid, a non-cationic lipid, a cholesterol-based lipid and a PEG-modified lipid.
163. The lipid nanoparticle of embodiment 161 or 162, wherein the cationic lipid is selected from cKK-E12, cKK-E10, OF-Deg-Lin and OF-02; the non-cationic lipid is selected from DOPE and DEPE; the cholesterol-based lipid is cholesterol; and the PEG-modified lipid is DMG-PEG-2K.
164. An immunogenic composition comprising the mRNA construct of embodiment 160 or the lipid nanoparticle of any of embodiments 161-163.
165. The immunogenic composition according to embodiment 164 comprising between 10 μg and 200 μg of the mRNA construct.
166. The immunogenic composition according to embodiment 165 comprising between 15 μg and 135 μg of the mRNA construct.
167. The immunogenic composition according to embodiment 166 comprising at least 20 μg of the mRNA construct.
168. The immunogenic composition according to embodiment 166 comprising at least 25 μg of the mRNA construct.
169. The immunogenic composition according to embodiment 166 comprising at least 35 μg of the mRNA construct.
170. The immunogenic composition according to embodiment 166 comprising at least 40 μg of the mRNA construct.
171. The immunogenic composition according to embodiment 166 comprising at least 45 μg of the mRNA construct.
172. The immunogenic composition according to embodiment 166 comprising 15 μg, 45 μg or 135 μg of the mRNA construct.
173. A method of treating or preventing a SARS-COV-2 infection, said method comprising administering to a subject an effective amount of the immunogenic composition of any one of embodiments 164 to 172.
174. The method of embodiment 173, wherein the immunogenic is administered to the subject at least twice.
175. The method of embodiment 174, wherein the period between administrations is at least 2 weeks, e.g., 3 weeks, or 1 month.
176. An immunogenic composition comprising at least two nucleic acids, for use in prophylaxis of an infection with SARS-COV-2, wherein a first nucleic acid comprises an optimized nucleotide sequence which encodes an amino acid sequence comprising a sequence selected from SEQ ID NO: 1, 2, 3, 4, 5, 8, 9, 10, 11, 12, 14, 15, 16, 17, 19, 20, 35, 66, 68, 70, 72, 74, 76, 78, 80, 82, 84. 86, 88, 90, 92, 94, 96, 98, 104, 106, 108, 110, 118, 120, 123, 125, 127, 129, 131, 133, 135, 137, 139 or 141, and wherein one or more further nucleic acid(s) comprise(s) an optimized nucleotide sequence which encodes an amino acid sequence comprising a sequence selected from SEQ ID NO: 151, 153, 155, 157, 159, 161, 163, 165, 167, 169 or 171.
177. An immunogenic composition comprising at least two nucleic acids, for use in prophylaxis of an infection with SARS-COV-2, wherein a first nucleic acid comprises an optimized nucleotide sequence which encodes an amino acid sequence comprising SEQ ID NO: 11, optionally wherein the optimized nucleotide sequence has the nucleic acid sequence of SEQ ID NO: 44, and
wherein one or more further nucleic acid(s) is (are) selected from:
   (a) a nucleic acid comprising an optimized nucleotide sequence which encodes an amino acid sequence comprising a sequence selected from SEQ ID NO: 157, optionally wherein the optimized nucleotide sequence has the nucleic acid sequence of SEQ ID NO 156, and
   (b) nucleic acid comprises an optimized nucleotide sequence that encodes an amino acid sequence comprising a sequence selected from SEQ ID NO: 163, optionally wherein the optimized nucleotide sequence has the nucleic acid sequence of SEQ ID NO: 162; and
   (c) nucleic acid comprises an optimized nucleotide sequence that encodes an amino acid sequence comprising a sequence selected from SEQ ID NO: 167, optionally wherein the optimized nucleotide sequence has the nucleic acid sequence of SEQ ID NO: 166; and
   (d) nucleic acid comprises an optimized nucleotide sequence that encodes an amino acid sequence comprising a sequence selected from SEQ ID NO: 171, optionally wherein the optimized nucleotide sequence has the nucleic acid sequence of SEQ ID NO 170.
178. The immunogenic composition according to embodiment 176 or embodiment 177, wherein the at least two nucleic acids are mRNA.
179. The immunogenic composition according to embodiment 178, wherein the first nucleic acid comprises an optimized nucleotide sequence which encodes an amino acid sequence comprising SEQ ID NO:11 and wherein the optimized nucleotide sequence has the nucleic acid sequence of SEQ ID NO: 148.
180. The immunogenic composition according to embodiments 176-178, wherein the nucleic acids are encapsulated in a lipid nanoparticle.
181. A method of treating or preventing a SARS-COV-2 infection, said method comprising administering to a subject an effective amount of the immunogenic composition of any one of embodiments 176-179.

### EXAMPLES

### Example 1. Generating Optimized Nucleotide Sequences

This example illustrates a process that results in optimized nucleotide sequences in accordance with the invention that are optimized to yield full-length transcripts during in vitro synthesis and result in high levels of expression of the encoded protein.

The process combines the codon optimization method of FIG. 1A with a sequence of filtering steps illustrated in FIG. 1B to generate a list of optimized nucleotide sequences. Specifically, as illustrated in FIG. 1A, the process receives an amino acid sequence of interest and a first codon usage table which reflects the frequency of each codon in a given organism (namely human codon usage preferences in the context of the present example). The process then removes codons from the first codon usage table if they are associated with a codon usage frequency which is less than a threshold frequency (10%). The codon usage frequencies of the codons not removed in the first step are normalized to generate a normalized codon usage table.

Normalizing the codon usage table involves re-distributing the usage frequency value for each removed codon; the usage frequency for a certain removed codon is added to the usage frequencies of the other codons with which the removed codon shares an amino acid. In this example, the re-distribution is proportional to the magnitude of the usage frequencies of the codons not removed from the table. The process uses the normalized codon usage table to generate a list of optimized nucleotide sequences. Each of the optimized nucleotide sequences encode the amino acid sequence of interest.

As illustrated in FIG. 1B, the list of optimized nucleotide sequences is further processed by applying a motif screen filter, guanine-cytosine (GC) content analysis filter, and codon adaptation index (CAI) analysis filter, in that order, to generate an updated list of optimized nucleotide sequences.

As illustrated in following examples, this process results in optimized nucleotide sequences encoding the amino acid sequence of interest. The nucleotide sequences yield full-length transcripts during in vitro synthesis and result in high levels of expression of the encoded protein (see Example 2).

### Example 2. Codon Optimization to Generate Nucleotide Sequences with a High CAI Score Improves Protein Yield

This example demonstrates that codon-optimized protein coding sequences with a codon adaptation index (CAI) of about 0.8 or higher outperform codon-optimized protein coding sequences with a CAI below 0.8.

Codon optimization was performed on a wild-type amino acid sequence of human erythropoietin (hEPO). hEPO is a protein hormone secreted by the kidney in response to low cellular oxygen levels (hypoxia). hEPO is essential for erythropoiesis, the production of red blood cells. Recombinant hEPO is commonly used in the treatment of anemia, a condition characterized by a low red blood cell or hemoglobin count, which can occur in subjects with chronic kidney disease or in subjects undergoing cancer chemotherapy.

Using different codon optimization algorithms, a total of 5 new codon-optimized nucleotide sequences encoding hEPO (#1 through #5) were generated. Nucleotide sequences #4 and #5 were generated according to a codon optimization method as illustrated in FIGS. 1A and 1B. As a reference, a nucleotide sequence with a codon-optimized hEPO coding sequence was provided that had previously been validated experimentally both in vitro and in vivo. The reference nucleotide sequence had been found to provide superior protein yield relative to the wild-type nucleotide sequence and other codon-optimized nucleotide sequences encoding the hEPO protein.

TABLE 5
hEPO-encoding nucleotide sequences

| | |
|---|---|
| SEQ ID NO: 112 | ATGGGTGTGCACGAATGTCCTGCTTGGCTGTGGCTCCTTCTCTC |
| | CCTGCTGTCCCTGCCTCTTGGACTCCCGGTGCTTGGAGCACCCC |
| | CGAGACTGATCTGCGACAGCAGGGTGCTCGAGCGCTACCTCCT |
| | GGAAGCCAAGGAAGCCGAAAACATCACTACTGGCTGCGCCGA |
| | ACACTGCTCCCTGAACGAGAACATCACCGTGCCGGACACCAAG |
| | GTCAACTTCTACGCGTGGAAGAGAATGGAGGTCGGACAGCAA |
| | GCCGTGGAAGTGTGGCAGGGACTTGCGCTCCTGTCGGAAGCCG |
| | TGCTGAGGGGACAAGCCCTGCTCGTGAACAGCTCACAGCCTTG |
| | GGAGCCCCTGCAGCTGCATGTCGACAAGGCCGTGTCCGGACTG |
| | CGCTCACTGACCACTCTGCTGAGGGCCTTGGGTGCCCAGAAAG |
| | AGGCTATTTCCCCACCGGATGCAGCCTCGGCAGCTCCTCTGCG |
| | GACCATTACGGCGGACACCTTTCGGAAGCTGTTCCGCGTCTAC |
| | AGCAATTTCCTCCGGGGGAAGTTGAAACTGTATACCGGCGAAG |
| | CCTGTCGGACTGGCGATCGCTGA |
| SEQ ID NO: 113 | ATGGGGGTTCATGAGTGCCCAGCTTGGCTTTGGCTCCTGCTCAG |
| | CTTGCTTAGTCTCCCTTTGGGCCTGCCCGTGCTGGGCGCCCCTC |
| | CACGCTTGATCTGTGACAGCAGGGTCTTGGAACGGTATTTGCTT |
| | GAAGCTAAAGAAGCTGAGAACATAACAACGGGATGTGCTGAA |
| | CATTGCTCCTTGAACGAAAACATCACAGTTCCCGACACAAAAG |
| | TCAATTTTTACGCATGGAAGCGGATGGAGGTTGGCCAGCAAGC |
| | TGTGGAGGTCTGGCAAGGGCTGGCTCTTCTCAGTGAAGCCGTG |
| | CTGCGCGGACAAGCACTCTTGGTGAACTCCAGCCAGCCCTGGG |
| | AGCCCCTTCAGCTCCATGTCGATAAAGCAGTTAGCGGCCTCCG |
| | ATCATTGACTACCCTCCTTAGGGCTTTGGGTGCACAAAAAGAG |
| | GCCATTTCACCACCGGACGCGGCAAGTGCTGCTCCGTTGCGAA |
| | CTATAACTGCTGACACCTTCCGGAAACTTTTTCGGGTATATTCC |
| | AACTTTCTCAGGGGGAAACTCAAGCTCTACACCGGCGAGGCGT |
| | GCCGAACTGGAGACCGCTGA |
| SEQ ID NO: 114 | ATGGGCGTACATGAATGCCCGGCATGGCTTTGGCTGCTGCTGT |
| | CCCTGCTGAGTTTGCCGCTGGGCCTCCCCGTCCTCGGCGCTCCC |
| | CCGAGACTCATTTGCGACTCTAGGGTCCTCGAACGCTATCTGCT |
| | GGAAGCAAAAGAAGCTGAGAACATAACTACAGGATGCGCTGA |
| | GCACTGTTCCTTGAATGAGAATATCACAGTACCTGACACTAAG |
| | GTGAATTTTTACGCATGGAAACGCATGGAAGTGGGTCAGCAGG |
| | CCGTGGAAGTGTGGCAGGGCCTGGCGCTGCTGTCCGAGGCTGT |
| | TCTTAGAGGCCAAGCCTTGTTGGTCAATTCCTCTCAACCCTGGG |
| | AGCCCCTCCAGCTGCATGTTGATAAAGCCGTCTCTGGTCTCCGG |
| | TCCCTTACCACCCTGCTCAGGGCACTTGGCGCACAGAAGGAAG |
| | CTATCTCCCCCCCAGACGCTGCCAGTGCCGCCCCCCTCCGGACT |
| | ATTACCGCCGATACTTTCAGGAAACTGTTTCGAGTCTATAGCAA |
| | TTTTCTCCGCGGGAAACTGAAGCTGTATACAGGTGAGGCCTGC |
| | AGGACAGGAGATCGCTGA |
| SEQ ID NO: 115 | ATGGGCGTGCACGAATGTCCTGCTTGGCTGTGGCTGCTGCTGA |
| | GTCTGCTGTCTCTGCCTCTGGGACTGCCTGTTCTTGGAGCCCCT |
| | CCTAGACTGATCTGCGACAGCAGAGTGCTGGAAAGATACCTGC |
| | TGGAAGCCAAAGAGGCCGAGAACATCACAACAGGCTGTGCCG |
| | AGCACTGCAGCCTGAACGAGAATATCACCGTGCCTGACACCAA |
| | AGTGAACTTCTACGCCTGGAAGCGGATGGAAGTGGGACAGCA |
| | GGCTGTGGAAGTTTGGCAAGGACTGGCCCTGCTGTCTGAAGCT |
| | GTTCTGAGAGGACAGGCTCTGCTGGTCAATAGCTCTCAGCCTT |
| | GGGAACCTCTCCAGCTGCATGTGGATAAGGCCGTGTCTGGCCT |
| | GAGAAGCCTGACAACACTGCTGAGAGCCCTGGGAGCCCAGAA |
| | AGAGGCCATTTCTCCACCTGATGCTGCCAGCGCTGCCCCTCTGA |
| | GAACAATCACCGCCGACACCTTCAGAAAGCTGTTCCGGGTGTA |
| | CAGCAACTTCCTGCGGGGCAAGCTGAAACTGTACACCGGCGAA |
| | GCCTGCAGAACCGGCGATAGATAA |
| SEQ ID NO: 116 | ATGGGGGTGCACGAGTGCCCTGCCTGGCTGTGGTTGCTGCTGT |
| | CCCTGCTGTCTCTGCCACTGGGACTGCCAGTGCTGGGAGCTCCA |
| | CCTAGGCTGATCTGCGACAGCCGGGTCCTGGAGAGGTACCTGC |
| | TCGAGGCCAAGGAGGCCGAGAACATTACCACAGGCTGCGCCG |
| | AGCACTGCAGCCTGAACGAGAACATTACAGTGCCCGATACAAA |
| | GGTGAACTTCTACGCCTGGAAGAGGATGGAGGTGGGCCAGCA |
| | GGCCGTGGAGGTGTGGCAGGGGCTGGCCCTGCTGAGCGAGGCC |
| | GTGCTGAGGGGCCAAGCCCTGCTGGTCAACAGCAGCCAGCCTT |
| | GGGAGCCCCTGCAGCTCCACGTGGACAAGGCTGTGTCTGGCTT |
| | GAGGTCTCTCACAACATTGCTGAGGGCCCTGGGCGCACAGAAA |
| | GAAGCTATCAGCCCACCTGATGCCGCTAGTGCCGCTCCACTGC |
| | GGACAATTACCGCCGATACCTTTAGAAAATTGTTCAGGGTCTA |
| | CTCCAACTTTTTGCGCGGGAAGCTGAAGCTCTATACCGGCGAG |
| | GCCTGCCGGACAGGGGACAGATGA |
| SEQ ID NO: 117 | ATGGGAGTGCACGAATGTCCTGCATGGCTCTGGCTCCTGCTGTC |
| | TCTCCTGAGCCTGCCACTGGGACTCCCAGTGCTGGGAGCACCC |
| | CCTAGGCTGATCTGCGATTCTCGGGTGCTGGAGCGCTACCTGCT |
| | CGAGGCTAAGGAGGCCGAGAATATCACTACTGGGTGTGCCGAA |
| | CACTGTAGCCTCAATGAAAACATTACAGTCCCAGATACCAAGG |
| | TGAACTTTTATGCATGGAAGAGGATGGAGGTCGGGCAGCAGGC |
| | AGTGGAGGTGTGGCAGGGACTGGCTCTGCTGTCCGAAGCCGTG |
| | CTCAGAGGTCAGGCCCTGCTGGTTAATTCCAGCCAGCCTTGGG |
| | AACCTCTGCAGCTGCATGTGGACAAGGCAGTGTCTGGCCTGAG |
| | ATCCCTTACTACACTGCTGAGAGCACTGGGGGCTCAGAAAGAA |
| | GCTATTTCCCCACCAGACGCCGCCTCAGCAGCACCTCTCCGGA |
| | CCATCACTGCTGACACCTTCCGCAAGCTCTTTAGGGTGTACTCC |
| | AACTTCCTGCGCGGGAAGCTCAAGCTGTACACCGGCGAAGCCT |
| | GCAGGACCGGGGATCGCTGA |

The characteristics of each of the 5 nucleotide sequences in terms of CAI, GC content, codon frequency distribution (CFD) as well as the presence of negative CIS elements and negative repeat elements is summarized in Table 6.

TABLE 6
Characteristics of the optimized nucleotide
sequences encoding hEPO

| | | | GC | | Negative | Negative |
|---|---|---|---|---|---|---|
| Nucleotide | | | content | CFD | CIS | repeat |
| Sequence | SEQ ID NO. | CAI | % | % | elements | elements |
| Reference | SEQ ID NO: | 0.79 | 61.06% | 3% | 0 | 0 |
| | 112 | | | | | |
| #1 | SEQ ID NO: | 0.69 | 54.12% | 2% | 0 | 0 |
| | 113 | | | | | |
| #2 | SEQ ID NO: | 0.76 | 56.23% | 1% | 0 | 0 |
| | 114 | | | | | |
| #3 | SEQ ID NO: | 0.90 | 57.28% | 0% | 0 | 0 |
| | 115 | | | | | |
| #4 | SEQ ID NO: | 0.89 | 60.95% | 0% | 0 | 0 |
| | 116 | | | | | |
| #5 | SEQ ID NO: | 0.86 | 59.56% | 0% | 0 | 0 |
| | 117 | | | | | |

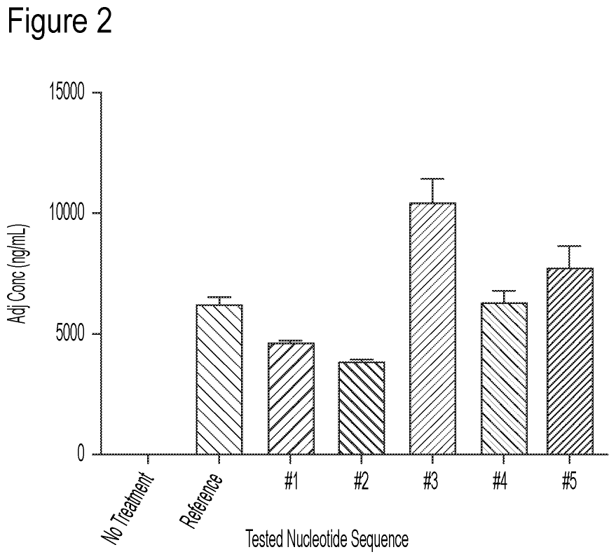

In order to test the protein yield from each of the codon-optimized sequences, 6 nucleic acid vectors were prepared each comprising an expression cassette that contained one of the 6 nucleotide sequences encoding the hEPO protein flanked by identical 3′ and 5′ untranslated sequences (3′ and 5′ UTRs) and preceded by an RNA polymerase promoter. These nucleic acid vectors served as templates for in vitro transcription reactions to provide 6 batches of mRNA containing the 6 codon-optimized nucleotide sequences (reference and nucleotide sequences #1 through #5). Capping and tailing were performed separately. Each of the capped and tailed mRNAs were separately transfected into a cell line (HEK293). Expression levels of the encoded hEPO protein was assessed by ELISA. The results of this experiment are summarized in FIG. 2.

As can be seen from FIG. 2, the highest level of expression was observed with nucleotide sequence #3, which yielded nearly twice as much hEPO protein as the experimentally validated reference nucleotide sequence. A trend towards higher protein yield could be observed for sequences depending on their CAI (cf. Table 6). Nucleotide sequence #3 with the highest protein yield had the highest CAI. The second and third highest yielding nucleotide sequences #4 and #5 had the third and fourth highest CAI. The lowest performing nucleotide sequences #1 and #2 also had the lowest CAI. Incidentally, these were also the nucleotide sequences with the lowest GC content. However, GC content alone was not determinative. The reference nucleotide sequence had the highest GC content (61%) of all tested codon-optimized sequences, but did not perform as well as nucleotide sequences #3, #4 and #5, all of which had a lower GC content. Notably, the lowest performing nucleotide sequences #1 and #2 also had a higher CFD.

Taken together, the data in this example demonstrate that codon optimization of a therapeutically relevant nucleotide sequence to achieve a CAI of about 0.8 or higher results in greater protein yield than, e.g., codon optimization to achieve a nucleotide sequence with the highest possible GC content.

### Example 3. Detection of Spike Proteins Produced Using Optimized Nucleic Constructs

This example demonstrates that optimized nucleotide sequences encoding a full-length SARS-COV-2 S protein are successfully expressed in cultured cells at high levels following transfection. It also demonstrates that the expressed protein is processed by the cells as expected.

Nucleic acid constructs comprising optimized nucleotide sequences encoding a full-length SARS-COV-2 S protein were generated according to a codon optimization method as illustrated in FIGS. 1A and 1B. The optimized nucleotide sequences are shown in Table 7.

TABLE 7
Nucleic acids comprising an optimized nucleotide
sequence encoding a SARS-CoV-2 S protein

| Construct | Optimized nucleic | Amino acid | |
|---|---|---|---|
| No. | acid sequence | sequence | Protein description |
| A | SEQ ID NO: 29 | SEQ ID NO: 1 | Native full-length SARS-CoV-2 spike |
| | | | protein |
| B | SEQ ID NO: 44 | SEQ ID NO: 11 | SARS-CoV-2 S protein that has been |
| | | | modified relative to naturally |
| | | | occurring SARS-CoV-2 spike protein to |
| | | | remove the furin cleavage site and to |
| | | | mutate residues 986 and 987 to proline |
| C | SEQ ID NO: 43 | SEQ ID NO: 10 | SARS-CoV-2 S protein that has been |
| | | | modified relative to naturally occurring |
| | | | SARS-CoV-2 spike protein to mutate |
| | | | residues 986 and 987 to proline |
| D | SEQ ID NO: 42 | SEQ ID NO: 9 | SARS-CoV-2 S protein that has been |
| | | | modified relative to naturally occurring |
| | | | SARS-CoV-2 spike protein to remove |
| | | | the furin cleavage site |

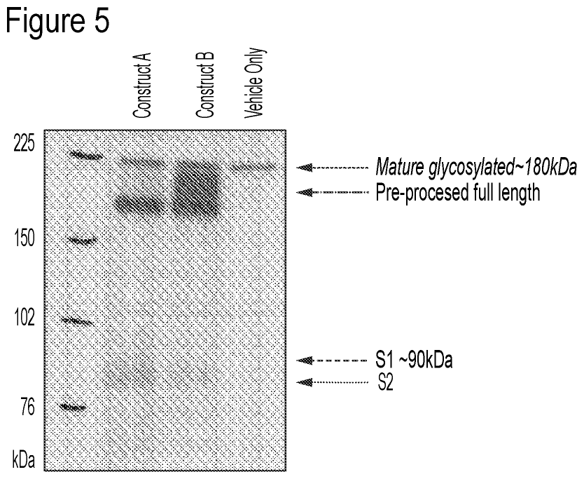

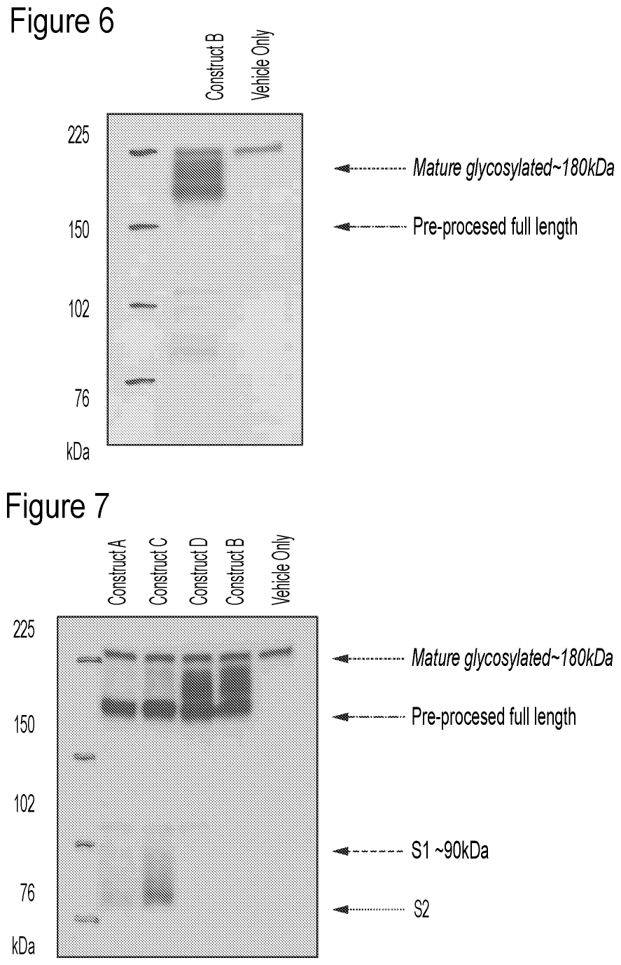

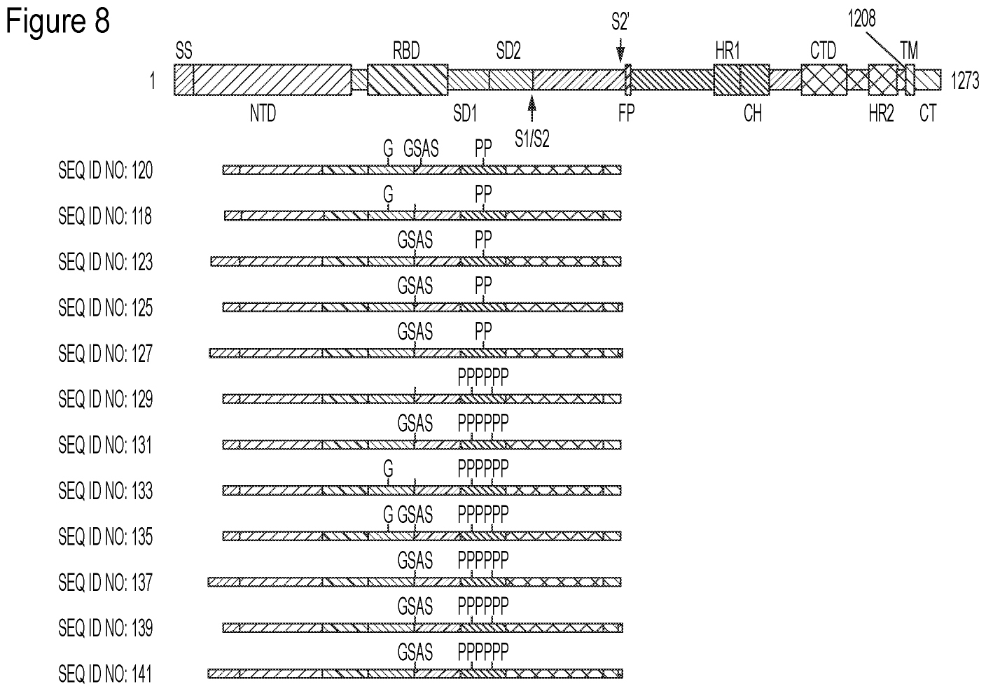

For transfection of cultured cells, 150 μL OptiMEM Reduced Serum Medium was added to a 1.5 mL Eppendorf tube, along with 0.5 μg (FIG. 7) or 1 μg (FIGS. 5 and 6) mRNA and 2.5 μL Lipofectamine 2000 for complexation of the mRNA to the transfection reagent. Each tube was gently mixed on a Vortex and spun briefly in a microcentrifuge to collect the contents. The complexes were incubated for 10±2 minutes at room temperature. Then the entire complex volume was carefully added to a well of a 12 well plate, so as not to disturb the HEK293 cell monolayer (5×10⁵ per well). The cells were returned to a 37° C. incubator and incubated for 18±2 hours prior to harvesting.

The contents of each well was harvested by removing the culture medium and adding 250 μL of CelLytic M (Sigma)+1× HALT. The cell suspension was left for 20 minutes on ice to allow the cells to fully lyse, before the lysates were collected in 1.5 mL Eppendorf tubes. The lysates were centrifuged at 13,000 RPM for 3 minutes to pellet the debris. The supernatants were transferred to clean 1.5 mL Eppendorf tubes. From this point forward, samples were always kept on ice.

For Western Blotting, 15 μL of each cell lysate was combined with 5 μL 4× Novex NuPAGE LDS Sample Buffer supplemented with 1× NuPage Sample Reducing Agent. The samples were incubated at 85° C. for 5 minutes, then cooled on ice. The entire sample volume was loaded into a Novex WedgeWell 12-well 6% tris-glycine mini gel with 3 μg I-56578SS/gel and run for 1-1.5 hour at 165V. A TransBlot Turbo with the PVDF transfer pack was used for transfer and the membranes were blocked in 0.2% iBlock (Thermo) with 0.05% Tween-20 in 1×PBS. The membranes were incubated for ≥1 hour with primary antibody (Anti-rabbit HRP #W401B) diluted as specified in blocking buffer. They were then washed twice with 1×TBST (Thermo). The membranes were then incubated for ≥1 hour with species-appropriate secondary antibody diluted 1:10,000 in blocking buffer. They were then washed four times with 1×TBST. The membranes were then develop using SuperSignal Pico West substrate on film.

Transfection of mRNAs containing the optimized nucleotide sequences described in Table 7 resulted in levels of protein expression in cultured HEK293 cells. FIGS. 5 and 6 show a ˜170-180 kDa band corresponding to a pre-processed full length S protein. FIG. 5 also shows the presence of S1 and S2 subunit bands, demonstrating that the native full length SARS-COV-2 S protein (Construct A) is processed correctly by the cells. A large band corresponding to fully glycosylated mature protein was observed when cells expressed construct B. Construct B encodes a variant SARS-COV-2 S protein that is modified relative to naturally occurring SARS-COV-2 spike protein to lack the furin cleavage site (and therefore is not cleaved to form the S1 and S2 subunits) and to contain prolines as residues 986 and 987 (thereby stabilizing the protein in its prefusion conformation).

FIG. 7 also shows the full length S protein band of ˜170-180 kDa. This band was observed with all 4 constructs tested. S1 and S2 subunit bands were detected with construct A and construct C. Construct C expresses a variant SARS-COV-2 S protein which is modified relative to naturally occurring SARS-COV-2 S protein to contain prolines as residues 986 and 987 (thereby stabilizing the protein in its prefusion conformation). Again, the fully glycosylated mature protein was detected as a strong band with construct B and construct D. Construct D encodes a variant SARS-COV-2 S protein that is modified relative to naturally occurring SARS-COV-2 S protein to lack the furin cleavage site (and therefore is not cleaved to form the S1 and S2 subunits).

This example demonstrates that optimized nucleic acid sequences encoding full length SARS-COV-2 S protein or variants thereof are expressed at high levels. It also demonstrates that the expressed protein is processed by the cells as expected.

### Example 4. Neutralizing Antibody Response to Immunization with Sequence-Optimized mRNAs Encoding a Full-Length Prefusion Stabilized SARS-COV-2 S Protein

This examples demonstrates that mRNAs comprising an optimized nucleotide sequence encoding a full-length prefusion stabilized SARS-COV-2 S protein are effective in inducing a neutralizing antibody response in mice.

Each of the four mRNAs containing the optimized nucleotide sequences described in Table 7 of Example 3 was encapsulated in lipid nanoparticles (LNPs). Groups of BALB/c mice were administered two immunizations at a 0.4 μg dose of one of the four formulations at a three week interval. Binding antibody activities in the serum samples were assessed via Enzyme-Linked Immunosorbent Assay (ELISA). To determine titers of neutralizing antibodies, a pseudovirus-based neutralization assay was used.

For the ELISA, 2019-nCOV Spike protein (S1+S2) ectodomain (Sino Biological, Cat #40589-V08B1) was used as substrate and coated at 2 μg/mL concentration in bicarbonate buffer overnight at 4° C. The plates were developed using colorimetric substrate, Sure Blue TMB 1-component (SERA CARE, KPL Cat #5120-0077), and stopped by Stop solution (SERA CARE Sure Blue, KPL Cat #5120-0024). The endpoint antibody titer for each sample was determined as the highest dilution which gave an OD value 3× higher than the background.

For the pseudovirus-based neutralization assay, serum samples were diluted 1:4 in medium (FluoroBrite phenol red free DMEM+10% FBS+10 mM HEPES+1% PS+1% Glutamax) and heat inactivated at 56° C. for 0.5 h. Further, 2-fold dilution series of the heat inactivated sera were prepared and mixed with the reporter virus particle (RVP)-GFP (Integral Molecular), diluted to contain 300 infectious particles per well and incubated for 1 h at 37° C. 96-well plates of 50% confluent 293T-hsACE2 clonal cells in 75 μL volume were inoculated with 50 μL of the serum/virus mixtures and incubated at 37° C. for 72h. At the end of the incubation, plates were scanned on a high-content imager and individual GFP expressing cells were counted. The inhibitory dilution titer (ID50) was reported as the reciprocal of the dilution that reduced the number of virus plaques in the test by 50%. ID50 for each test sample was interpolated by calculating the slope and intercept using the last dilution with a plaque number below the 50% neutralization point and the first dilution with a plaque number above the 50% neutralization point. ID50 Titer=(50% neutralization point−intercept)/slope.

All four mRNA formulations induced similar levels of binding antibodies 14 days after the first vaccination, and the responses were further enhanced one week after the second dose at Day 28. On Day 35, the geometric mean titers (GMTs) for neutralizing antibodies as determined by pseudovirus neutralization assay were 152 for construct A, 354 for construct B, 195 for construct C, and 1005 for construct D. The neutralizing potential of construct D variant was slightly trending higher than construct B.

Serological antibody titers detected for binding in ELISA were not predictive of neutralizing titers determined by pseudovirus. Some mice in the construct A and construct C groups did not seroconvert in the neutralization assay but their endpoint titration titers in ELISA were comparable to the others in the group. Constructs B and D were likely comparable in immunogenicity for induction of neutralizing antibodies.

This example demonstrates that mRNAs comprising an optimized nucleotide sequence encoding a full-length prefusion stabilized SARS-COV-2 S protein are more effective in inducing neutralizing antibody titers than an mRNA that encodes a native full-length SARS-COV-2 S protein. Blocking the furin cleavage site in addition to mutating residues 986 and 987 to proline adds another layer for prevention of prefusion to postfusion conversion. Considering the importance of the pre-fusion conformation, construct B (encoding a SARS-COV-2 S protein that has been modified relative to naturally occurring SARS-COV-2 S protein to remove the furin cleavage site and to mutate residues 986 and 987 to proline) was selected for further preclinical evaluations.

### Example 5. Preparation of mRNA-Encapsulating Lipid Nanoparticles

An mRNA comprising an optimized nucleotide sequence encoding a full-length SARS-COV-2 S protein that has been modified relative to naturally occurring SARS-COV-2 spike protein to remove the furin cleavage site and to mutate residues 986 and 987 to proline was synthesized in vitro. The mRNA was prepared using a template plasmid comprising the following nucleic acid sequence operable linked to an RNA polymerase promoter sequence:

| (SEQ ID NO: 149) | |
|---|---|
| 1 | GGACAGATCG CCTGGAGACG CCATCCACGC TGTTTTGACC TCCATAGAAG |
| 51 | ACACCGGGAC CGATCCAGCC TCCGCGGCCG GGAACGGTGC ATTGGAACGC |
| 101 | GGATTCCCCG TGCCAAGAGT GACTCACCGT CCTTGACACG ATGTTCGTCT |
| 151 | TCCTCGTGCT GCTCCCACTC GTTTCTTCCC AGTGTGTCAA CCTGACAACT |
| 201 | AGGACTCAGC TGCCACCAGC CTACACCAAC TCCTTCACCA GAGGCGTGTA |
| 251 | TTACCCAGAC AAGGTGTTTA GAAGCAGCGT GCTGCACTCT ACCCAGGACC |
| 301 | TCTTTCTGCC CTTTTTCAGC AACGTGACAT GGTTTCACGC AATTCACGTG |
| 351 | TCCGGCACTA ATGGCACAAA GCGGTTCGAC AATCCAGTCC TGCCTTTCAA |
| 401 | CGATGGCGTC TACTTTGCAT CTACTGAGAA ATCCAATATC ATTAGGGGAT |
| 451 | GGATCTTCGG CACAACCCTG GATTCTAAGA CCCAGAGCCT GCTGATCGTC |
| 501 | AACAACGCCA CAAACGTGGT CATTAAGGTT TGCGAGTTTC AGTTCTGTAA |
| 551 | CGATCCTTTT CTGGGCGTGT ATTATCATAA GAACAATAAG AGCTGGATGG |
| 601 | AGTCCGAGTT TAGAGTGTAT AGCTCTGCAA ATAATTGTAC CTTTGAGTAC |
| 651 | GTGAGCCAGC CCTTTCTGAT GGACCTGGAG GGAAAACAAG GAAACTTCAA |
| 701 | AAACCTGCGG GAATTCGTTT TCAAAAACAT CGACGGCTAT TTCAAGATCT |
| 751 | ATAGCAAGCA TACCCCAATC AACCTCGTGA GGGACCTCCC CCAGGGCTTT |
| 801 | AGCGCACTGG AGCCACTGGT TGACCTGCCT ATCGGCATTA ATATCACAAG |
| 851 | ATTTCAGACC CTGCTGGCAC TGCATAGAAG CTATCTGACC CCTGGAGACT |
| 901 | CCTCTAGTGG GTGGACTGCC GGCGCCGCTG CCTACTATGT GGGCTATCTG |
| 951 | CAGCCACGGA CATTCCTGCT GAAATACAAT GAGAACGGGA CAATCACAGA |
| 1001 | TGCTGTTGAT TGCGCACTCG ACCCCCTGTC CGAGACAAAG TGCACTCTCA |
| 1051 | AGAGCTTTAC CGTCGAGAAG GGCATCTATC AGACCTCAAA CTTCAGGGTG |
| 1101 | CAGCCCACAG AATCTATCGT GCGCTTCCCT AATATCACTA ACCTGTGTCC |
| 1151 | TTTCGGTGAA GTGTTCAACG CCACCAGGTT TGCTAGCGTG TATGCCTGGA |
| 1201 | ACAGGAAGAG GATCTCTAAC TGCGTCGCCG ACTATTCCGT GCTGTATAAC |
| 1251 | AGCGCCTCCT TCTCCACATT CAAATGCTAT GGAGTGAGCC CGACAAAACT |
| 1301 | GAACGATCTC TGCTTTACAA ATGTCTACGC CGACTCTTTT GTGATCAGAG |
| 1351 | GGGACGAGGT CCGGCAGATC GCACCAGGAC AGACAGGCAA GATTGCTGAC |
| 1401 | TACAACTATA AGCTGCCTGA CGACTTCACA GGATGTGTGA TCGCATGGAA |
| 1451 | CTCAAACAAT CTGGACTCCA AAGTCGGGGG CAACTATAAT TACCTGTATC |
| 1501 | GCCTGTTCCG GAAGTCCAAC CTGAAGCCCT TCGAGAGGGA CATCAGTACA |
| 1551 | GAGATCTATC AGGCTGGCTC CACCCCTTGC AATGGCGTCG AAGGCTTTAA |
| 1601 | TTGTTATTTT CCCCTGCAGT CTTACGGGTT TCAGCCTACT AATGGAGTTG |
| 1651 | GGTACCAGCC ATACAGAGTG GTCGTGCTCA GCTTCGAGCT CCTGCATGCT |
| 1701 | CCAGCTACAG TTTGCGGGCC AAAGAAGTCC ACTAACCTGG TGAAGAATAA |
| 1751 | GTGCGTCAAC TTCAACTTTA ACGGGCTCAC CGGCACCGGC GTGCTGACTG |
| 1801 | AGAGCAACAA GAAGTTTCTG CCATTTCAAC AGTTTGGACG GGACATTGCC |
| 1851 | GACACCACCG ATGCCGTTCG GGATCCACAG ACCCTGGAAA TTCTGGACAT |
| 1901 | TACACCGTGC AGCTTCGGGG GCGTGAGCGT GATCACACCC GGAACCAATA |
| 1951 | CAAGCAACCA GGTTGCCGTC CTGTATCAGG ATGTCAATTG CACAGAAGTG |
| 2001 | CCAGTTGCTA TCCACGCAGA CCAGCTGACT CCCACATGGC GGGTGTATAG |
| 2051 | CACCGGATCC AACGTGTTTC AGACCCGCGC CGGATGTCTC ATTGGGGCCG |
| 2101 | AGCACGTGAA TAACAGCTAC GAGTGCGACA TCCCCATTGG CGCCGGCATT |
| 2151 | TGTGCGTCTT ACCAGACTCA GACCAACTCT CCTGGCTCCG CCTCTTCCGT |
| 2201 | TGCTAGTCAG TCTATTATTG CCTATACCAT GAGCCTCGGA GCTGAGAATA |
| 2251 | GCGTGGCCTA CTCCAATAAT TCCATCGCAA TCCCTACTAA CTTCACTATT |
| 2301 | TCTGTGACCA CCGAGATCCT GCCTGTGTCT ATGACTAAGA CTAGCGTTGA |
| 2351 | TTGTACCATG TATATTTGTG GCGACTCTAC CGAATGTTCT AACCTGCTGC |
| 2401 | TTCAGTACGG CTCATTTTGC ACACAGCTGA ACAGAGCCCT GACTGGGATC |
| 2451 | GCTGTGGAGC AGGACAAGAA CACACAGGAG GTGTTTGCAC AGGTGAAGCA |
| 2501 | GATCTATAAG ACCCCTCCTA TTAAGGATTT CGGCGGATTC AATTTCTCAC |
| 2551 | AGATTCTGCC AGACCCCAGT AAGCCTTCCA AGAGGAGCTT CATCGAGGAT |
| 2601 | CTCCTGTTTA ACAAGGTGAC CCTGGCAGAC GCCGGCTTTA TTAAGCAATA |
| 2651 | TGGGGATTGC CTGGGCGACA TTGCTGCCAG AGACCTGATT TGCGCCCAGA |
| 2701 | AATTCAATGG CCTCACAGTG CTGCCACCTC TGCTGACCGA CGAGATGATC |
| 2751 | GCTCAATACA CTAGCGCACT GCTGGCCGGA ACCATCACAT CAGGCTGGAC |
| 2801 | CTTCGGGGCC GGAGCAGCAC TGCAGATTCC ATTCGCCATG CAGATGGCCT |
| 2851 | ATAGATTCAA CGGCATTGGC GTCACACAGA ACGTGCTGTA CGAAAACCAG |
| 2901 | AAGCTCATCG CTAACCAGTT TAATTCCGCA ATTGGAAAGA TCCAAGATTC |
| 2951 | ACTCAGCTCA ACCGCCTCTG CACTCGGAAA GCTGCAGGAC GTGGTCAACC |
| 3001 | AGAATGCTCA GGCCCTGAAC ACACTCGTCA AGCAGCTGTC CTCTAACTTT |
| 3051 | GGCGCTATCA GCTCCGTTCT GAACGACATT CTGAGCCGCC TGGATCCCCC |
| 3101 | AGAGGCTGAA GTCCAGATTG ACCGCCTGAT TACCGGCCGG CTGCAGTCTC |
| 3151 | TGCAAACATA CGTGACCCAG CAGCTGATCA GAGCAGCCGA GATCCGGGCA |
| 3201 | TCCGCAAATC TGGCAGCAAC TAAGATGAGC GAATGCGTGC TGGGCCAGTC |
| 3251 | CAAGCGGGTG GACTTTTGTG GCAAGGGCTA CCACCTGATG AGCTTCCCCC |
| 3301 | AGAGCGCCCC ACATGGCGTT GTTTTTCTGC ACGTGACCTA TGTCCCTGCT |
| 3351 | CAGGAAAAGA ACTTTACAAC TGCTCCTGCT ATCTGCCATG ACGGCAAGGC |
| 3401 | CCACTTCCCA CGGGAGGGAG TGTTTGTGTC CAATGGCACA CACTGGTTCG |
| 3451 | TGACCCAGAG GAACTTCTAT GAACCCCAGA TCATCACCAC TGACAATACC |
| 3501 | TTCGTGTCTG GAAATTGCGA CGTCGTGATC GGCATCGTTA ACAACACCGT |
| 3551 | GTACGACCCT CTCCAGCCAG AGCTGGACTC CTTTAAGGAG GAACTGGATA |
| 3601 | AGTATTTTAA GAACCACACA AGCCCAGATG TGGATCTCGG GGACATCTCC |
| 3651 | GGAATTAACG CCTCCGTGGT GAATATCCAG AAGGAGATTG ACCGCCTAAA |
| 3701 | TGAAGTTGCC AAGAACCTCA ATGAGTCTCT GATTGATCTG CAGGAACTGG |
| 3751 | GCAAGTATGA GCAGTATATC AAATGGCCCT GGTACATTTG GCTGGGGTTT |
| 3801 | ATCGCCGGAC TGATTGCCAT CGTCATGGTG ACCATCATGC TGTGTTGCAT |
| 3851 | GACCTCCTGT TGTTCCTGTC TGAAGGGCTG CTGTAGTTGC GGCTCTTGCT |
| 3901 | GTAAATTCGA CGAAGATGAT AGCGAGCCCG TGCTGAAGGG CGTGAAGCTG |
| 3951 | CATTATACCT GACGGGTGGC ATCCCTGTGA CCCCTCCCCA GTGCCTCTCC |
| 4001 | TGGCCCTGGA AGTTGCCACT CCAGTGCCCA CCAGCCTTGT CCTAATAAAA |
| 4051 | TTAAGTTGCA TCAAGCT |

Template-dependent RNA synthesis of unmodified nucleotides yielded a polynucleotide with the nucleic acid sequence of SEQ ID NO: 147 which comprises the optimized nucleic acid sequence of SEQ ID NO: 148. In a multi-step, enzyme-catalyzed process, the final mRNA product was synthesized, which was purified to remove enzyme reagents and prematurely aborted synthesis products (“shortmers”).

The final mRNA had the structural elements shown in Table 4. The SARS-COV-2 S protein coding sequence is flanked by 5′ and 3′ untranslated regions (UTRs) of 140 and 105 nucleotides, respectively. The mRNA also contains a 5′ cap structure consisting of a 7-methyl guanosine (m7G) residue linked via an inverted 5′5′ triphosphate bridge to the first nucleoside of the 5′ UTR, which is itself modified by 2′Oribose methylation. The 5′ cap is essential for initiation of translation by the ribosome. The entire linear structure is terminated at the 3′ end by a tract of approximately 100 to 500 adenosine nucleosides (polyA). The polyA region confers stability to the mRNA and is also thought to enhance translation. All of these structural elements are naturally occurring components which are required for the efficient translation of the SARS-COV-2 spike mRNA.

The purified mRNA was encapsulated in lipid nanoparticles (LNPs) comprising a proprietary cationic lipid, a non-cationic lipid (DOPE), a cholesterol-based lipid (cholesterol) and a PEG-modified lipid (DMG-PEG-2K). The final mRNA-LNP formulation was an aqueous suspension.

### Example 6. Induction of a Neutralizing Antibody Response in Mice

This example demonstrates that an immunogenic composition of LNP-encapsulated mRNA comprising an optimized nucleotide sequence encoding a full-length pre-fusion stabilized SARS-COV-2 S protein induces a robust response of binding and neutralizing antibodies against the SARS-COV-2 S protein in mice.

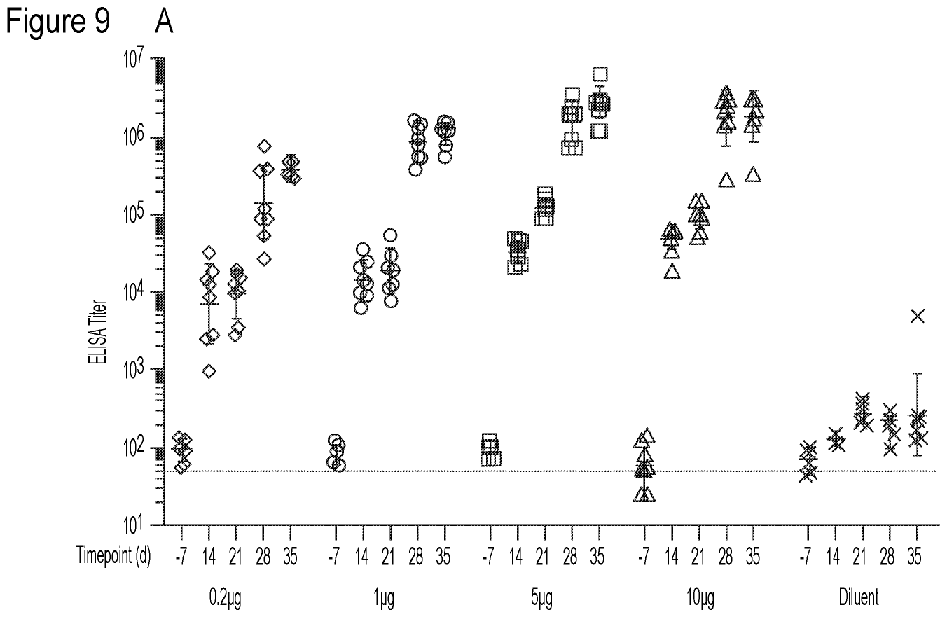

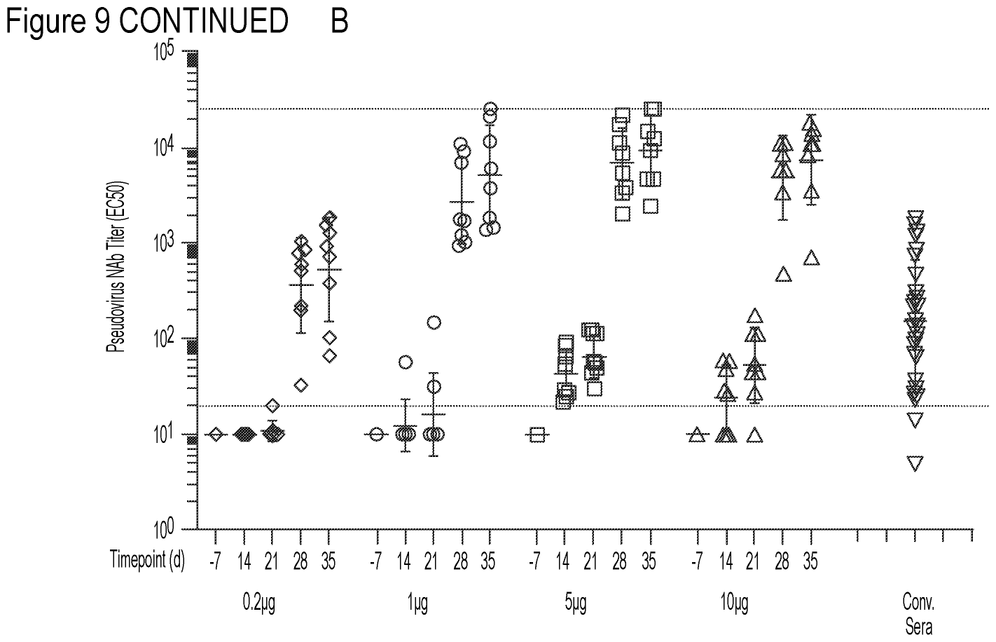

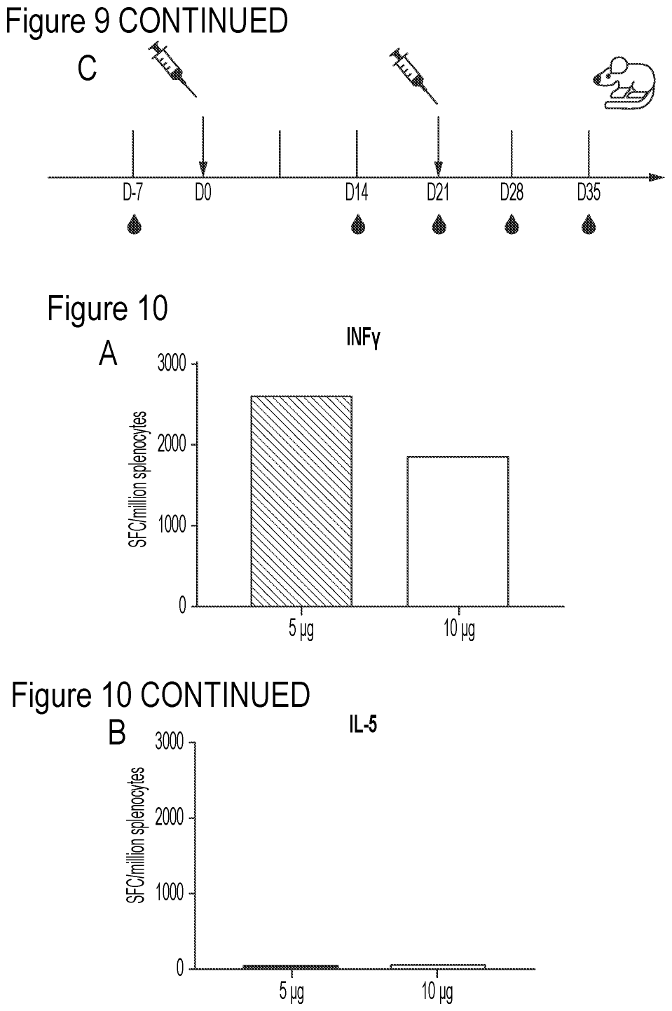

The LNP formulation prepared in Example 5 was used to immunize mice twice by intramuscular injection (IM), at Day 0 and Day 21 (see FIG. 9C). Four groups of eight 6-8 week-old BALB/c mice were immunized with 0.2 μg, 1 μg, 5 μg or 10 μg mRNA per dose, respectively. A fifth group of mice (which served as a negative control) received only the diluent of the mRNA-LNP composition. Seven days (Day 7) prior to immunization a blood sample was taken from each mouse to determine the baseline level of antibodies against the SARS-COV-2 S protein. Additional blood samples were taken at Day 14, Day 21, Day 28 and Day 35. The mouse experiments were carried out in compliance with all pertinent US National Institutes of Health regulations and approval from the Animal Care and Use Committee of Covance Inc, Denver, PA.

An ELISA assay was used to determine the antibody titer against SARS-COV-2 S protein. 96-well plates were coated with commercially available SARS-COV-2 S protein (SinoBio), incubated with serially diluted mouse sera from Day −7, Day 14, Day 21, Day 28 and Day 35 and probed with secondary antibodies to detect bound total mouse IgG.

To determine titers of neutralizing antibodies, a pseudovirus-based assay was used. 39 individual conversion serum samples from COVID-19 patients with mild, strong and severe symptoms served as positive control. Serum samples were diluted 1:4 in medium (FluoroBrite phenol red free DMEM+10% FBS+10 mM HEPES+1% PS+1% Glutamax) and heat inactivated at 56° C. for 30 minutes. A further 2-fold, 9-point serial dilution series of the heat inactivated sera was performed in the same media. Diluted serum samples were mixed with a volume of Reporter Virus Particle (RVP)-Green Fluorescent Protein (GFP) (Integral Molecular) diluted to contain ˜300 infectious particles per well and incubated for 1 hour at 37° C. 96-well plates of ˜50% confluent 293T-hsACE2 clonal cells in 75 μL volume were inoculated with 50 μL of the serum+virus mixtures in singleton and incubated at 37° C. for 72h. At the end of the 72-hour incubation, plates were scanned on a high-content imager and individual GFP expressing cells counted. The neutralizing antibody titers are reported as the reciprocal of the dilution that reduced the number of virus plaques in the test by 50% (see FIG. 9B).

The results of this mouse immunization experiment are summarized FIGS. 9A and 9B. Even after a single shot, a robust antibody response was observed by ELISA at Day 14 for all tested doses (see FIG. 9A). A second shot resulted in a significant boost of the antibody response and dramatically improved the titer of neutralizing antibodies (see FIG. 9B). Administration of two doses of 1 μg, 5 μg or 10 μg mRNA resulted in comparable antibody titers as determined by ELISA at Day 35. As can be seen in FIG. 9B, two doses of 0.2 μg mRNA were slightly less effective in inducing neutralizing antibodies at Day 35, whereas two doses of 1 μg, 5 μg or 10 μg mRNA induced comparable titers of antibodies at Day 35, exceeding the titer of neutralizing antibodies observed in the conversion sera of human patients previously infected with SARS-CoV-2.

This example demonstrates that the immunogenic composition tested in this example induces a robust neutralizing antibody response after two doses. The magnitude of the response was dose-dependent. The results indicate that the immunogenic composition can induce neutralizing antibody titers comparable to those in convalescent human patients.

### Example 7. Induction of a Th1-Biased T Cell Response in Mice

A vaccine that promotes Th1-biased immunity is typically more protective against viral pathogens than a vaccine that does not. The secretion of Th1 cytokines such as IFN-γ activates cytotoxic T lymphocytes (CTL), a sub-group of T cells, which can induce the death of cells infected with viruses. This example demonstrates that the immunogenic composition tested in Example 6 induces a Th1-biased T cell response in mice.

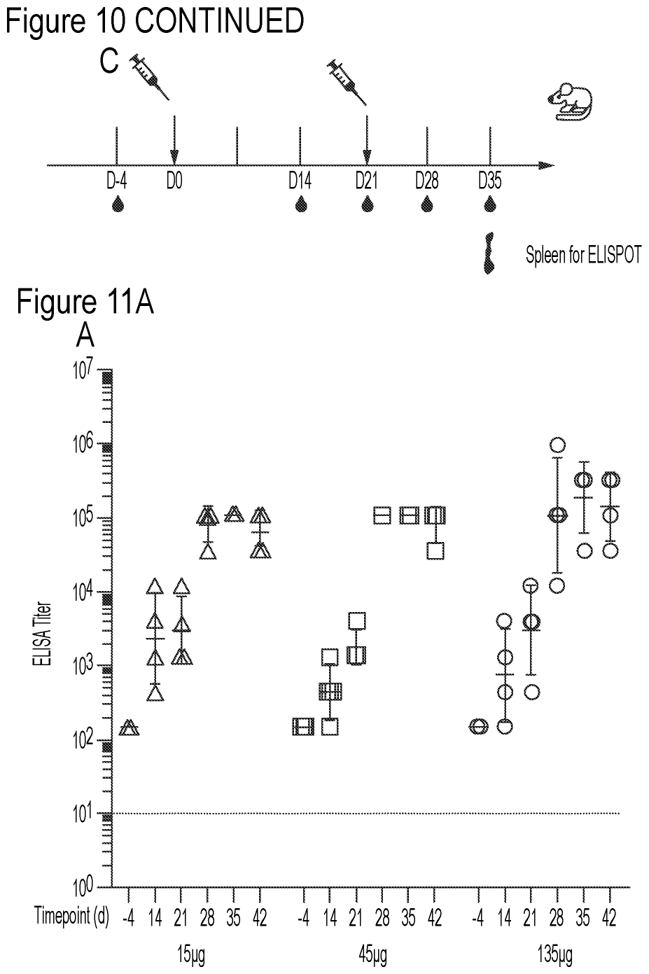

To further assess the quality of the immune response of the vaccine tested in Example 6, the experiment described in that example was repeated by immunizing groups of mice twice by IM injection with 5 μg or 10 μg mRNA, respectively. Blood was sampled on days Day-4 (baseline), Day 14, Day 21, Day 28 and Day 35 (see FIG. 10C). The mouse experiments were carried out in compliance with all pertinent US National Institutes of Health regulations and approval from the Animal Care and Use Committee of Covance Inc, Denver, PA. The mice were sacrificed on Day 35, and their spleens were removed. The isolated spleens were homogenized and splenocytes isolated as described below. IFN-γ and IL-5 secretion by peptide-stimulated splenocytes was determined by ELISPOT assay.

Harvested spleens were stored in a 5 mL of chilled medium on ice. Just prior to processing the spleens were placed into a sterile petri dish containing medium. The back of a 10 cc syringe plunger was used to homogenize the spleens. The homogenate was passed through a filter and transferred into a sterile tube. The homogenate was then be pelleted by centrifugation at 1200 rpm for 8-10 minutes. Supernatant was gently poured off and edge of tube blotted with a clean paper towel. ACK lysis buffer was added to lyse the red blood cells and cells were incubated at room temperature for 5 min. The tube was centrifuged at 1200 rpm for 8-10 minutes. Supernatants were poured off and pellet resuspended in 2 mM L-Glutamine CTL-Test Media. The suspensions were filtered into new 15 mL conical tubes. The cells were maintained at 37° C. in humidified incubator, 5% CO2 until use.

Solution with PepMix™ SARS-COV-2 (Spike Glycoprotein, Cat #PM-WCPV-S-1) peptide pool 1 and peptide pool 2 were prepared using test medium. Final concentration of each peptide in the assay was 2 μg/ml. As a positive control, 1 μg/ml of ConA in test medium were used. These antigen/mitogen solutions were plated at 100 μL/well. The plates containing the antigen/mitogen solutions were placed into a 37° C. incubator for 10-20 minutes before plating cells to ensure the pH and temperature were optimal for cells. The cell concentration was adjusted to the desired concentration. 0.3×10⁶/100 μl/well splenocytes were added to the plates with the antigen/mitogen solution. Once completed, the plate was gently taped and placed into a 37° C. humidified incubator, 5% CO2 and incubated overnight. Plates were washed 2× with PBS and then 2× with 0.05% Tween-PBS, 200 μL/well.

Mouse IFN-γ/IL-5 Double-Color enzymatic ELISPOT kits (CTL Shaker Heights, Cleveland,) were used according to the manufacture's protocol. Detection solution was prepared per manufacturer's instructions and 80 μL was added to each well. The plates were then incubated at RT for 2 hrs. Plates were washed 3× with 0.05% Tween-PBS, 200 μL/well. Tertiary solution at 80 μL/well was added and plates will be incubated at RT for 30 min. Plates were washed 2× with 0.05% Tween-PBS, and then 2× with distilled water, 200 μL/well each time. Developer Solution was added to wells at 80 μL/well and incubated at RT for 15 min. Reaction was stopped by gently rinsing membrane with tap water three times. Plates were air-dried and scanned using a CTL analyzer. The number of cytokine producing cells per million cells is reported (see FIG. 10).

As can be seen from FIG. 10A, splenocytes isolated at Day 35 from mice immunized twice with either 5 μg or 10 μg of mRNA secreted large amounts of the Th1 cytokine IFN-γ. As can be seen from FIG. 10B, these cells did not, however, secrete detectable amounts of the Th2 cytokine IL-5.

This example demonstrates that the tested immunogenic composition is effective in inducing a Th1-biased T cell response in mice, indicating that vaccination with this immunogenic composition can induce a CTL response that recognizes and eliminates SARS-COV-2-infected cells.

### Example 8. Induction of a Neutralizing Antibody Response in Cynomolgus Monkeys

This example demonstrates that an immunogenic composition of LNP-encapsulated mRNA comprising an optimized nucleotide sequence encoding a full-length pre-fusion stabilized SARS-COV-2 S protein induces a robust response of binding and neutralizing antibodies against the SARS-COV-2 S protein in cynomolgus monkeys.

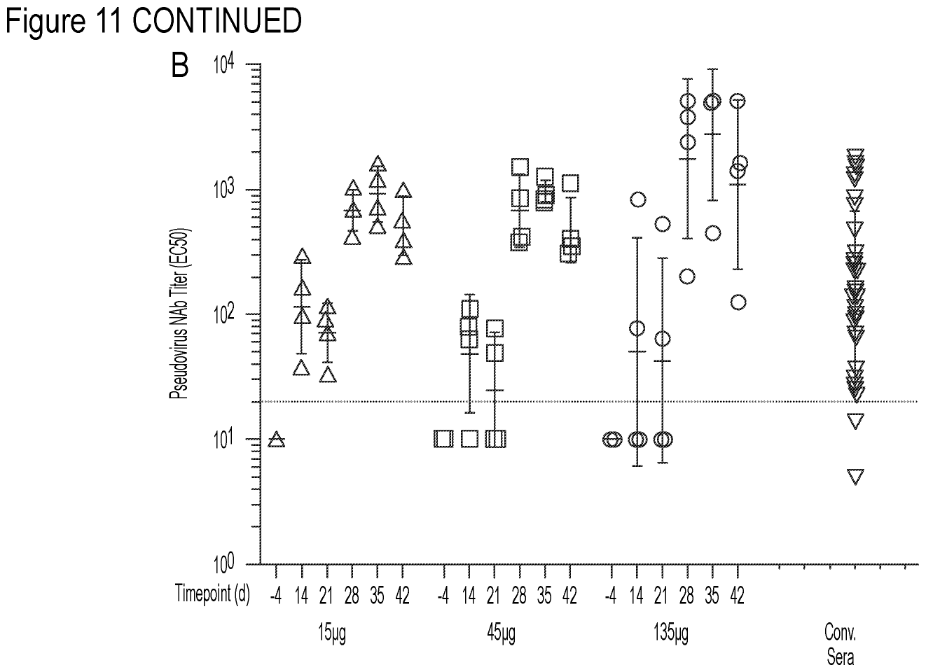

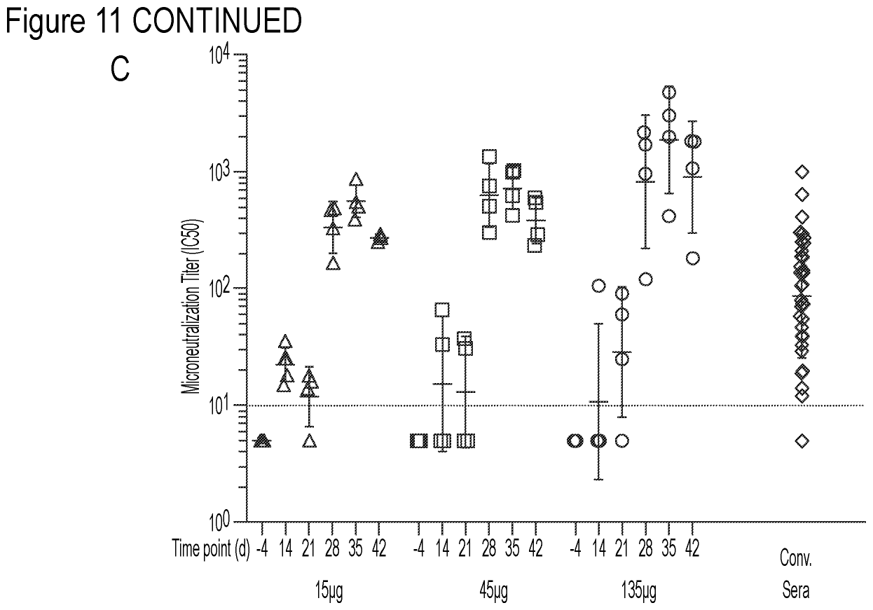

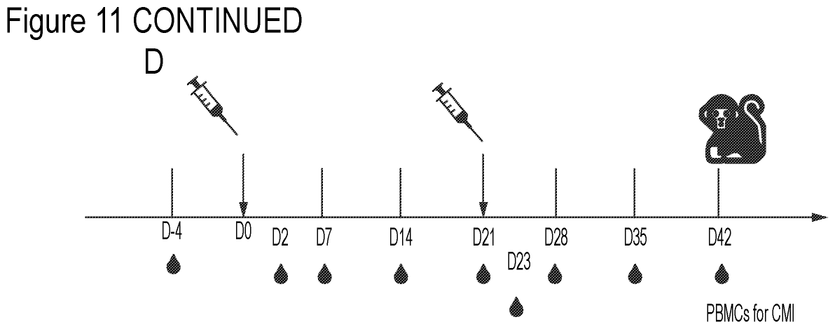

The LNP formulation prepared in Example 5 was used to immunize monkeys twice by IM administration, at Day 0 and Day 21 (see FIG. 11D). Three groups of four 3-4 year-old cynomolgus monkeys were immunized with 15 μg, 45 μg or 135 μg mRNA per dose, respectively. Four days (Day −4) prior to immunization a blood sample was taken from each monkey to determine the baseline level of antibodies against the SARS-COV-2 S protein. Additional blood samples were taken at Day −4, Day 2, Day 7, Day 14, Day 21, Day 23, Day 28 and Day 35 and Day 42. Cynomolgus monkey experiments were carried out in compliance with all pertinent US National Institutes of Health regulations and approval from the Animal Care and Use Committee of the New Iberia Research Center.

An ELISA assay was used to determine the antibody titers against SARS-COV-2 S protein in the blood samples obtained from the cynomolgus monkeys. 39 individual serum samples from COVID-19 patients with mild, strong and severe symptoms served as positive control. Nunc microwell plates were coated with SARS-COV S-GCN4 protein (GeneArt, expressed in Expi 293 cell line) at 0.5 μg/ml in PBS overnight at 4° C. Plates were washed 3 times with PBS-Tween 0.1% before blocking with 1% BSA in PBS-Tween 0.1% for 1 hour. Samples were plated with 1:450 initial dilution followed by 3-fold, 7-point serial dilution in blocking buffer. Plates were washed 3 times after 1-hour incubation at room temperature before adding 50 ul of 1:5000 Rabbit anti-human IgG (Jackson Immuno Research) to each well. Plates were incubated at room temperature for 1 hr and washed 3×. Plates were developed using Pierce 1-Step Ultra TMB-ELISA Substrate Solution for 6 minutes and stopped by TMB STOP solution. Plates were read at 450 nm in SpectraMax plate reader. Antibody titers were reported as the highest dilution that is equal to 0.2 OD cutoff.

Titers of neutralizing antibodies in the serum of the cynomolgus monkeys were determined using a pseudovirus-based assay. 39 individual conversion serum samples from COVID-19 patients with mild, strong and severe symptoms served as positive control. Serum samples were diluted 1:4 in media (FluoroBrite phenol red free DMEM+10% FBS+10 mM HEPES+1% PS+1% Glutamax) and heat inactivated at 56° C. for 30 minutes. A further, 2-fold, 9-point, serial dilution series of the heat inactivated serum was performed in media. Diluted serum samples were mixed with a volume of reporter virus particle (RVP)-GFP (Integral Molecular) diluted to contain ˜300 infectious particles per well and incubated for 1 hour at 37° C. 96-well plates of ˜50% confluent 293T-hsACE2 clonal cells in 75 μL volume were inoculated with 50 μL of the serum+virus mixtures in singleton and incubated at 37° C. for 72h. At the end of the incubation, plates were scanned on a high-content imager and individual GFP expressing cells counted. The neutralizing antibody titers are reported as the reciprocal of the dilution that reduced the number of virus plaques in the test by 50% (see FIG. 11B).

In addition, the microneutralization titer of each monkey sample was determined, using the 39 human conversion sera as positive controls. Vero E6 cells were seeded into 96-well flat bottom cell culture plates at a concentration of 2×10⁴ cells in 0.1 mL per well one day before use. On the day of the experiment, starting at a 1:10 dilution, 2-fold serial dilutions of heat-inactivated monkey or human sera were incubated with SARS-COV-2 virus (e.g., isolate USA-WA1/2020 [BEI Resources; catalog #NR-52281] in a 37° C. incubator for 60±5 minutes. Then the growth medium was aseptically removed from the Vero E6 cells and the test samples (sera and virus) were added to the Vero E6-seeded plates and incubate in a 37° C. incubator for 30±5 minutes. Subsequently, 100 μL of growth medium was added to all wells of all the plates without removing the existing inoculum. The plates were then placed back into the incubator and incubated for 2 days. Two days post infection, the cells were fixed and stained with primary antibody (SARS-COV anti-nucleoprotein mouse monoclonal antibody (SinoBio catalog #40143-MM05 or equivalent) and then with HRP-tagged secondary antibody (Horseradish peroxidase (HRP)-conjugated goat anti-mouse immunoglobulin G (IgG) antibody (Jackson ImmunoResearch Laboratories, catalog #115-035-062 or equivalent).

The results of these assays are summarized in FIG. 11. Even at the lowest tested mRNA dose of 15 μg, a robust binding and neutralizing antibody response was observed after two shots (see FIGS. 11A and 11B). Administration of two doses of 15 μg, 45 μg or 135 μg mRNA resulted in comparable antibody titers as determined by ELISA at Days 28, 35 and 42 (see FIG. 11A). Two doses of 15 μg or 45 μg mRNA also yielded comparable levels of neutralizing antibodies at these days (see FIG. 11B). Two doses of 135 μg mRNA induced titers of antibodies at Days 28, 35 and 42 that exceeded the titers of neutralizing antibodies observed in conversion sera of human patients infected with SARS-COV-2. The microneutralization titer assay provided similar results, with 15 μg and 45 μg mRNA doses resulting in comparable titers, and the 135 μg dose exceeding the titers observed in conversion sera of human patients infected with SARS-COV-2 (see FIG. 11C).

This example demonstrates that the tested immunogenic composition induces a robust neutralizing antibody response even at the lowest dose of 15 μg after two shots, when the period between administrations is at least 2 weeks (in particular about 3 weeks). The data support the use of the test composition in human patients to induce a protective neutralizing antibody response.

### Example 9. Induction of a Th1-Biased T Cell Response in Cynomolgus Monkeys

This example demonstrates that the immunogenic composition tested in Example 8 induces a Th1-biased T cell response in cynomolgus monkeys.

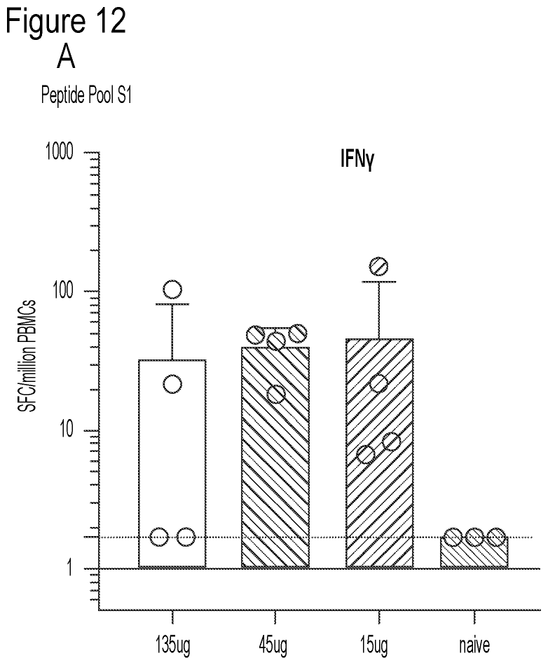

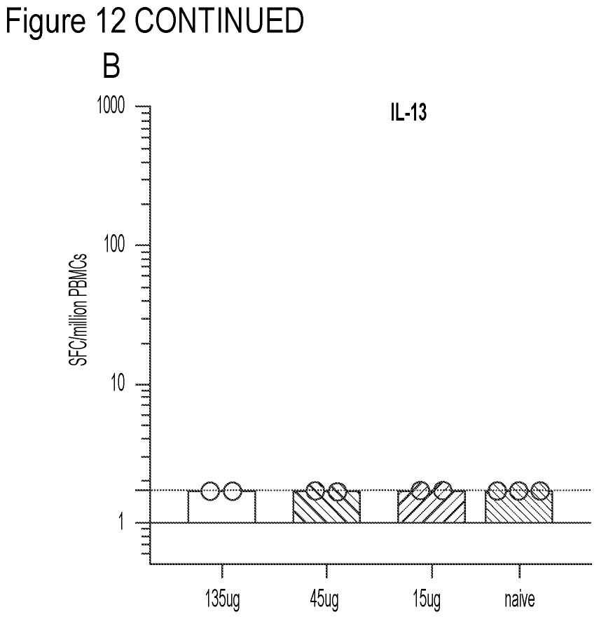

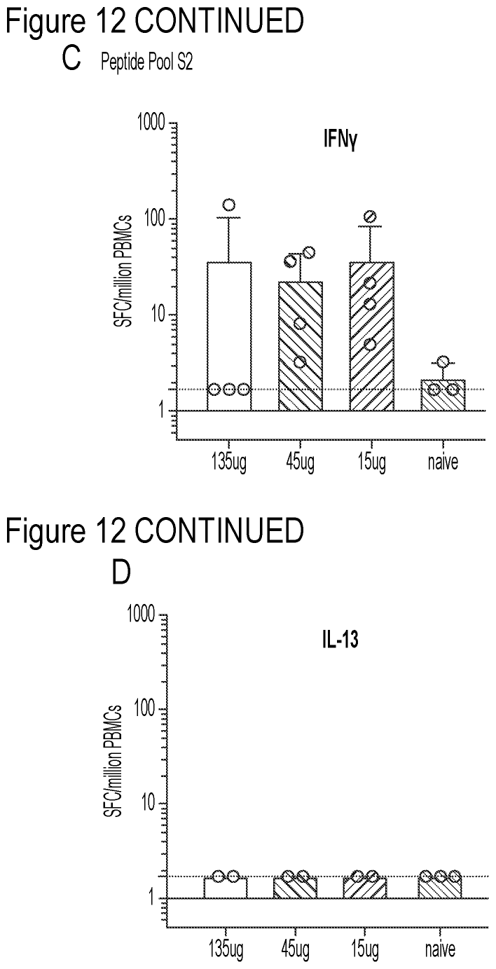

To further assess the quality of the immune response of the vaccine tested in Example 8, PBMCs were isolated as cynomolgus blood samples. Isolated PBMCs were stored in cryovials. T cell responses were assessed by determining IFN-γ and IL-13 secretion by peptide-stimulated PBMC using ELISPOT assays. Naïve PBMCs served as a control to establish baseline levels of IFN-γ or IL-13 secretion in non-activated, non-stimulated cells. The results are summarized in FIG. 12.

To perform the assays, complete medium for monkey PBMCs (DMEM1640+10% heat-inactivated FCS) was prewarmed in a 37° C. water bath. PBMCs cryovials were quickly thawed in a 37° C. water bath, and their content was slowly transferred dropwise into the prewarmed medium in conical tubes. The tubes were then centrifuged at 1500 RPM for 5 mins. The cell pellets were washed once with prewarmed complete medium, and re-pelleted at 1500 RPM for 15 min. The supernatant was discarded, and PBMCs were resuspended with complete medium and counted using a Guava cell counter.

Monkey IFN-γ ELISPOT kit (CTL, cat #3421M-4APW) and IL-13 ELISPOT kit (CTL, cat #3470M-4APW) were used to determine the levels of IFN-γ and IL-13 secretion by peptide-stimulated PBMCs. The precoated plates provided with the kits were washed 4 times with sterile PBS and then blocked with 200 μl/well complete medium. The blocking step was performed in a 37° C. incubator for at least 30 minutes. PepMix™ SARS-COV-2 (JPT Cat #PM-WCPV-S-1) peptide pool 1 and pool 2 were used as recall antigens at a final concentration of 2 μg/ml per peptide in the assay. 2 μg/ml of Concanavalin A (Sigma, cat #C5275) was used as a positive control. 50 μl of recall antigen and 300,000 PBMCs in 50 μl were added to each well for stimulation. The plates were then placed in a 37° C., 5% CO2 humidified incubator for 24 hours. Following the 24 hour incubation, the plates were washed 5 times with PBS. 100 μl/well of biotinylated anti-IFN-γ or anti-IL-13 detection antibodies (1 μg/ml) prepared in PBS containing 1% fetal calf serum were added, and the plates were incubated for 2 hours at room temperature. The plates were then washed 5 times with PBS as before and incubated for 1 hr at room temperature with 100 μl/well of streptavidin at a dilution of 1:1000 in PBS containing 1% fetal calf serum. The plates were again washed 5 times with PBS and developed using 100 μl/well BCIP/NBT substrate solution until the spots were visible. Color development was stopped by washing the plates in tap water. The plates were then dried overnight, scanned, and spots were counted using a CTL analyzer. The data are reported as spot forming cells (SFC) per million PBMCs (see FIG. 12).

As can be seen from FIGS. 12A (peptide pool S1) and 12C (peptide pool S2), PBMCs isolated at Day 42 from monkeys immunized twice with a dose of 15 μg, 45 μg or 135 μg mRNA secreted large amounts of the Th1 cytokine IFN-γ in response to stimulation with peptides derived from the SARS-COV-2 S protein. In contrast, these cells secreted only baseline amounts of the Th2 cytokine IL-13 in response to peptide stimulation (see FIGS. 12B (peptide pool S1) and 12D (peptide pool S2)).

This example demonstrates that the tested immunogenic composition is effective in inducing a Th1-biased T cell response in cynomolgus monkeys, indicating that vaccination with this immunogenic composition can induce a CTL response in humans that recognizes and eliminates SARS-COV-2-infected cells.

### Example 10. Dose Modelling

This example demonstrates that low mRNA doses of the immunogenic composition tested in Examples 6 and 8 are effective in yielding neutralizing antibody titers that are significantly higher than corresponding titers observed in a control panel of convalescent sera from COVID-19 patients.

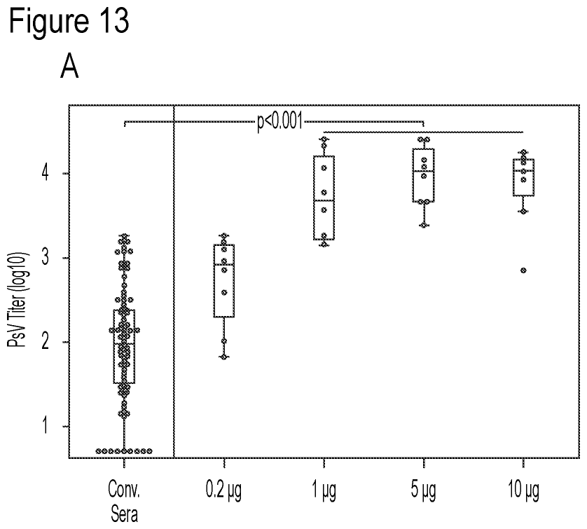

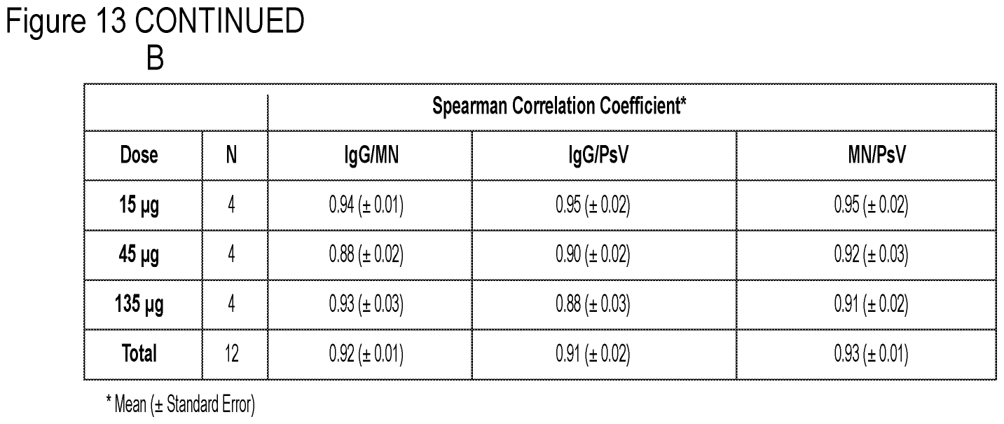

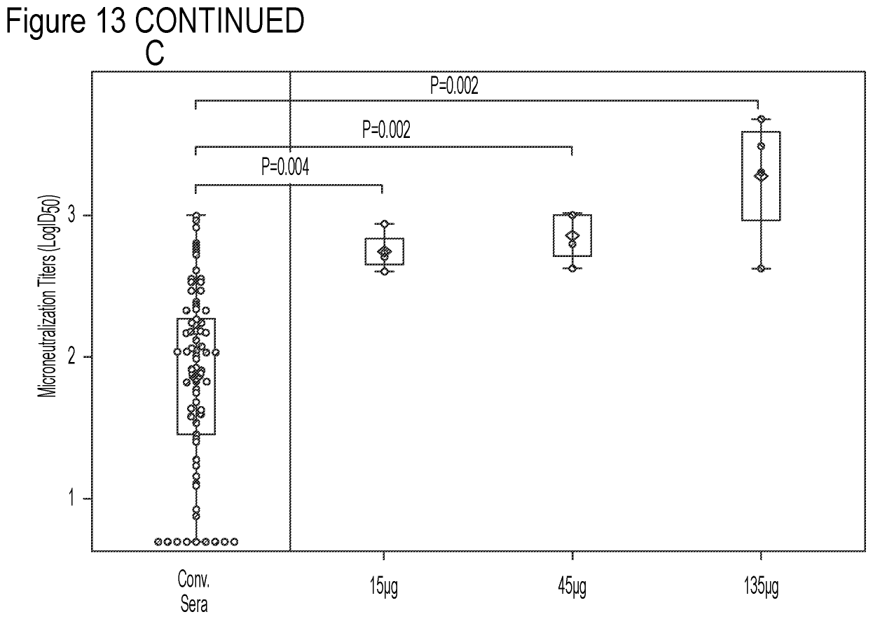

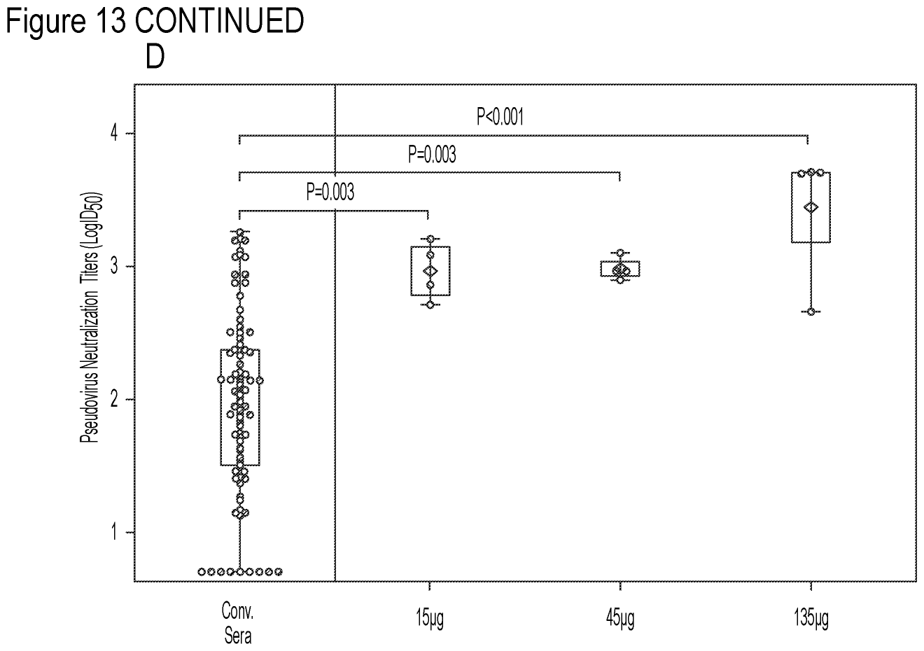

There were no statistically significant differences in pseudovirus neutralization titers on Day 35 between 1 μg, 5 μg and 10 μg groups of immunized mice described in Example 6, suggesting a dose-saturation effect beyond 1 μg of mRNA comprising the tested optimized nucleotide sequence encoding a full-length pre-fusion stabilized SARS-COV-2 S protein. Peak pseudovirus neutralization titers on Day 35 in mice were significantly higher than corresponding titers observed in the control panel of convalescent sera from COVID-19 patients (see FIG. 13A).

The results from both the pseudovirus neutralization assay and the microneutralization assay for the cynomolgus monkey experiments described in Example 8 were highly correlated (FIG. 13B). Regardless of the dose levels, Day 35 pseudovirus and microneutralization titers were about 130-fold higher than that of pre-immune animals. Further statistical analysis of a complete data set with 93 convalescent sera from COVID-19 patients revealed that the titers obtained with mRNA doses of 15 μg, 45 μg and 135 μg, respectively, were significantly higher than corresponding titers observed in the convalescent human sera (all P values were less than 0.005; FIGS. 13C and 13D).

This example supports an mRNA dose range of 10 μg to 200 μg for human clinical trials that investigate the safety and efficacy of the immunogenic composition prepared in Example 5. Indeed, a dose between 15 μg and 45 μg may be sufficient to induce an effective neutralizing antibody response, while being well-tolerated at the same time.

### Example 11. Immunogenicity of mRNAs Encoding Full-Length Prefusion Stabilized SARS-CoV-2 S Proteins

This example demonstrates that an mRNA encoding a SARS-COV-2 S protein that has been modified relative to naturally occurring SARS-COV-2 S protein to remove the furin cleavage site and to mutate residues 986 and 987 to proline (2P/GSAS) is more effective in eliciting a neutralizing antibody response than mRNA encoding other full-length prefusion stabilized SARS-COV-2 S protein.

To determine the impact of mutations that stabilize the SARS-COV-2 S protein in its prefusion confirmation on immunogenicity, seven mRNA constructs—a wild-type SARS-COV-2 S protein (WT) and corresponding prefusion stabilized SARS-COV-2 S proteins (2P, GSAS, 2P/GSAS, 2P/GSAS/ALAYT, 6P and 6P/GSAS, respectively)—were formulated in a lipid nanoparticle (LNP) as mRNA vaccines as described in Example 5. WT, 2P/GSAS, 2P, GSAS, correspond to constructs A-D in example 3 respectively. 2P/GSAS/KLHYT is a SARS-COV-2 S protein mutated to remove a furin cleavage site, to replace residues 986 and 987 with proline and to mutate the ER retrieval signal, which has the optimized nucleic acid sequence of SEQ ID NO: 124 and an amino acid sequence of SEQ ID NO: 125. 6P is a SARS-COV-2 S protein mutated to replace residues 817, 892, 899, 942, 986 and 987 with proline, which has the optimized nucleic acid sequence of SEQ ID NO: 128 and an amino acid sequence of SEQ ID NO: 129. 6P/GSAS is a SARS-COV-2 S protein mutated to remove a furin cleavage site and to replace residues 817, 892, 899, 942, 986 and 987 with proline, which has the optimized nucleic acid sequence of SEQ ID NO: 130 and an amino acid sequence of SEQ ID NO: 131.

Two animal models were used for the immune assessment. BALB/c mice were administered two immunizations at a three-week interval with a 0.4 μg per dose of each of five formulations (WT, 2P, GSAS, 2P/GSAS, 2P/GSAS/ALAYT). In parallel, non-human primates (NHPs) were immunized using the same immunization schedule at 5 μg per dose of six S mRNA vaccines (2P, GSAS, 2P/GSAS, 2P/GSAS/ALAYT, 6P and 6P/GSAS).

To evaluate for functional antibodies, e.g., nAbs titers, the ability of immune sera to neutralize the infectivity of GFP reporter pseudoviral particles (RVP) in HEK-293T cells stably over-expressing human ACE2 was tested. RVPs expressing SARS COV-2 S protein are capable of a single round of infection, indicated by GFP expression upon entry. Neutralizing potency was determined as the serum dilution which can achieve 50% inhibition of RVP entry (ID50). In addition, Enzyme-Linked Immunosorbent Assay (ELISA) titers were evaluated using a recombinant soluble S-protein trimerized by GCN4 helix bundle as antigen.

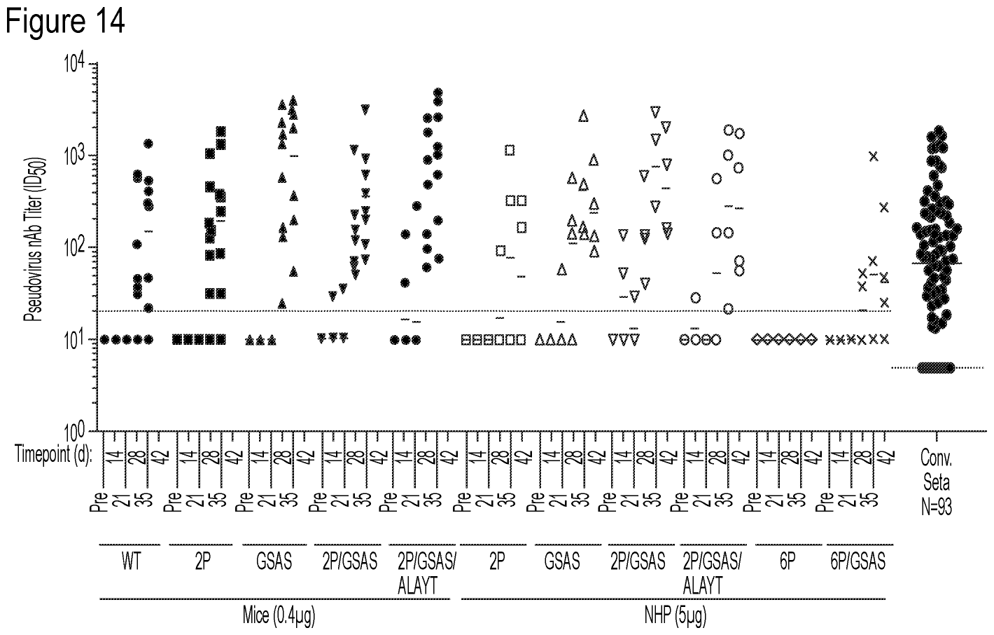

Although a few animals developed neutralizing titers at Day 14 after the first immunization, the titers were in general low. Expectedly, the majority of test animals developed neutralizing titers after the second immunization (FIG. 14). On Day 35, the geometric mean titers (GMTs) with the 95% confidence interval (95% CI) for pseudoviral (PsV) nAb titers in mice were 152 (36; 645) for WT, 195 (44; 870) for 2P, 1005 (261; 3877) for GSAS, 354 (129; 976) for 2P/GSAS and 940 for 2P/GSAS/ALAYT. There was a trend for higher GMTs, especially at Day 35 and Day 42, for the three constructs with GSAS mutations when compared to those of WT and 2P constructs.

In NHPs, diverse neutralizing titers were observed within each group even after the second immunization (FIG. 14). 2P and 6P/GSAS vaccines showed lower immunogenicity than other constructs with GMTs at Day 35 of 78 and 10, respectively. The 6P vaccine failed to elicit any detectable neutralizing titers. Consistent with the observations in the mouse study, all GSAS constructs with the exception of 6P/GSAS induced higher neutralizing titers after the second dose, with GMTs (95% CI) at D35 recorded as 425 (48; 3769) for GSAS, 772 (116; 5121) for 2P/GSAS, 280 (11; 6970) for 2P/GSAS/ALAYT, as compared to those of the 2P vaccine group. The trending of GMTs in both mice and NHPs suggested superior immunogenicity for 2P/GSAS to other constructs. Moreover, the peak PsVNa titers (Day 35) for the 2P/GSAS variant in mice and NHPs were comparable or higher than the titers observed in a panel of 93 convalescent sera from COVID-19 patients.

This example demonstrates that the GSAS mutation is beneficial for vaccine immunogenicity. The 2P mutation, which was introduced for stabilization of prefusion form of S protein, appeared beneficial in the context of the GSAS mutation, while ALAYT showed less impact on immunogenicity, especially in NHPs, in the context of 2P/GSAS. Accordingly, this example provides further confirmation that an optimized mRNA encoding a SARS-COV-2 S protein that has been modified relative to naturally occurring SARS-COV-2 S protein to remove the furin cleavage site and to mutate residues 986 and 987 to proline can be more effective in inducing neutralizing antibodies than mRNAs encoding other prefusion stabilized SARS-COV-2 S protein.

### Example 12. Protective Efficacy in Syrian Golden Hamsters

This example demonstrates that an immunogenic composition of LNP-encapsulated mRNA comprising an optimized nucleotide sequence encoding a SARS-COV-2 S protein that has been modified relative to naturally occurring SARS-COV-2 S protein to remove the furin cleavage site and to mutate residues 986 and 987 to proline can have protective efficacy in an animal model of COVID-19 by reducing viral infection of the lung and preventing lung pathology.

SARS-COV-2 infection in Syrian golden hamster is a pathology model, where the viral infection is associated with high levels of virus replication with peak titers in the lungs and nasal epitheliums at 2 day post infection (DPI), histopathological evidence of disease in lungs at 7 DPI, and about 8-15% weight loss around 7 DPI.

To evaluate the potential of the LNP formulation prepared in Example 5 to protect against viral infection and disease, Syrian golden hamsters were immunized with four vaccine formulation dose levels of 0.15, 1.5, 4.5 or 13.5 μg per dose, either per a single IM immunization at D21 or two IM administrations at Day 0 and Day 21. Animals were challenged at Day 49 via intranasal (IN) inoculation of SARS-COV-2 and monitored for clinical manifestations of disease as body weight loss at 8 DPI. Lungs and nasal tissues were harvested at 4 or 7 DPI for histopathology, and for quantification of viral replication by subgenomic RNA RT-PCR assays.

The LNP formulation of Example 5 induced robust dose-dependent neutralizing antibody responses after the first vaccination, which were significantly enhanced by the second immunization. After the first immunization, all animals, except for the 0.15 μg dose group, developed neutralizing antibodies recorded as plaque reduction neutralization titers (PRNT) against wild-type SARS-COV-2 virus. Day 35 PRNT50 GMTs for single-dose immunization schedules were 237, 410 and 711 for 1.5, 4.5 and 13.5 μg dose respectively, while corresponding values for two-dose groups were 3219, 2446 and 3219. Despite the observed trend towards higher titers with increasing dose, the differences between titers in the 1.5, 4.5 and 13.5 μg groups were not statistically significant.

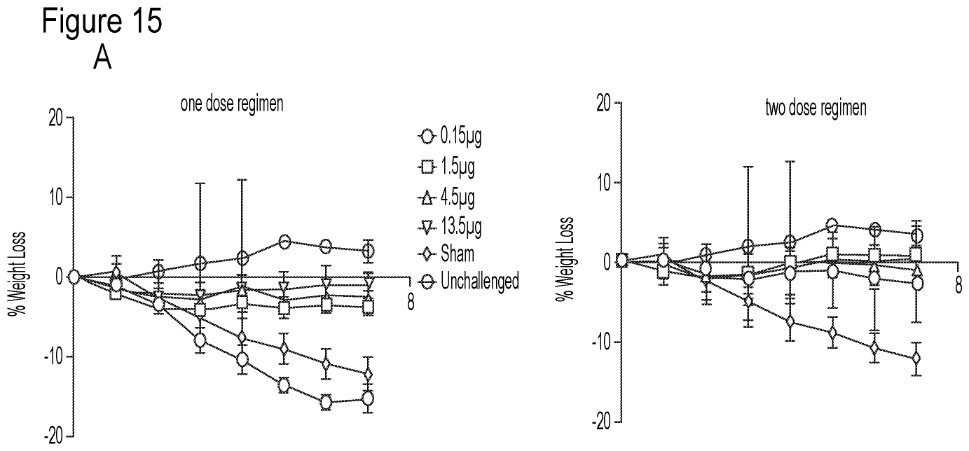

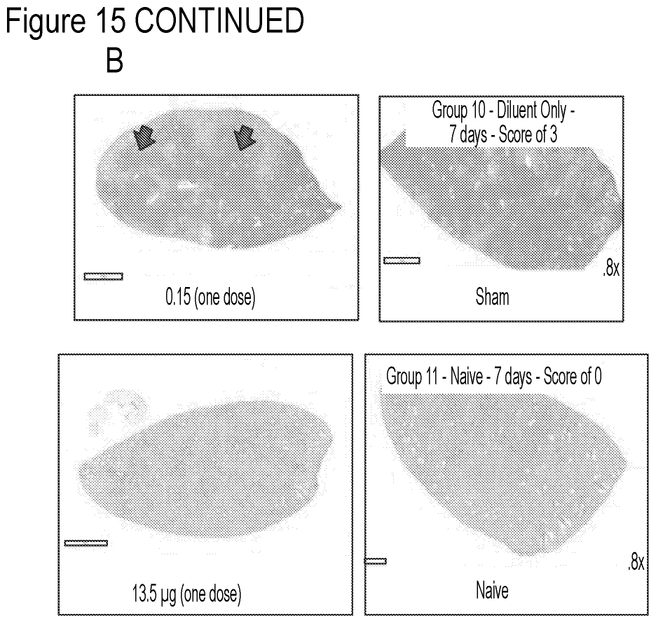

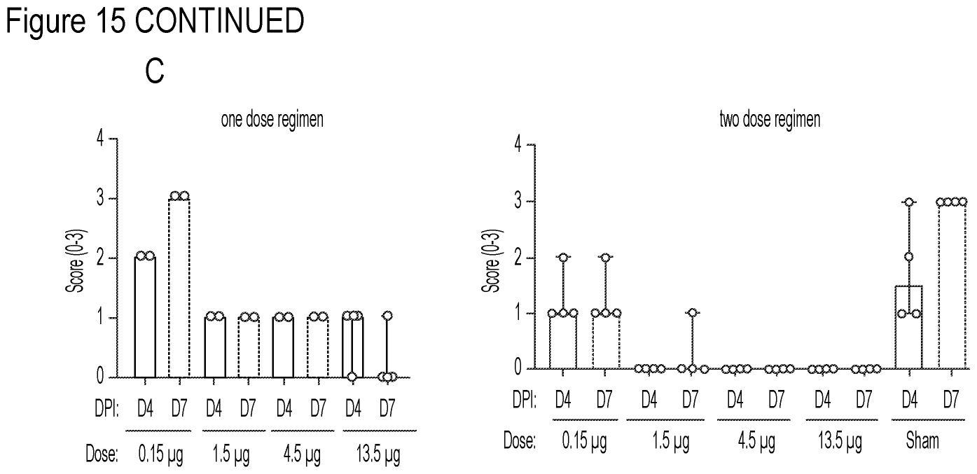

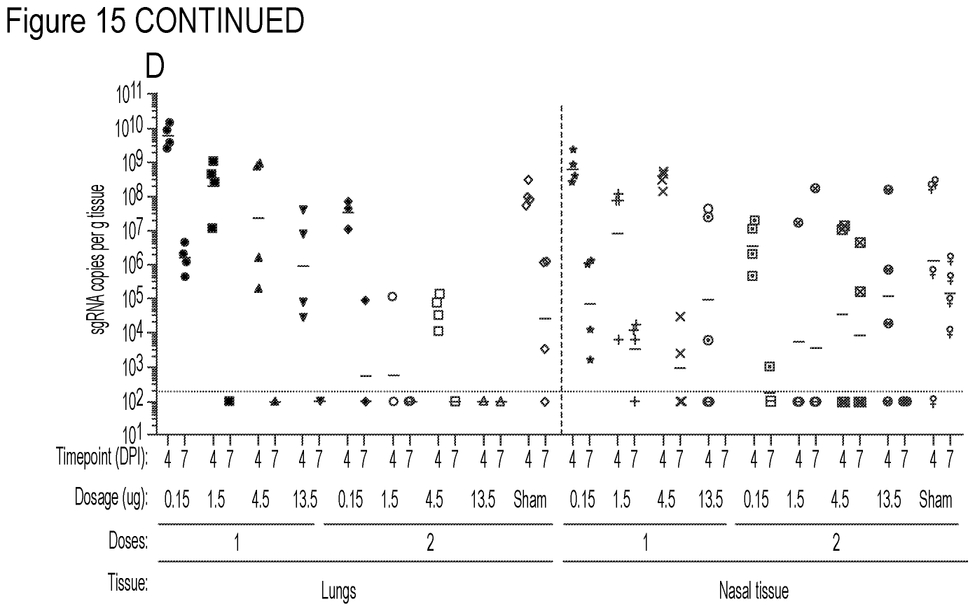

To test the protective effects of vaccination, all groups were challenged intranasally. The body weight for each animal was monitored daily for 7 days (FIG. 15a). Sham (diluent) vaccinated animals were observed with most significant weight loss, with more than 10% loss at 7 DPI. The vaccination regimens of 1.5, 4.5, and 13.5 μg, regardless of one-dose or two-dose regimens, protected animals against body weight loss, with most animals experiencing less than 5% loss, with the loss mostly peaking around 2-3 DPI. There was no significant difference for the weight comparison among these groups. The only group experiencing a similar degree of weight loss, compared to that of sham group, was the 0.15 μg dose group with single immunization.

To assess the pathology caused by viral infection, lung samples were harvested from 4 animals of each group on either 4 or 7 DPI, and the fixed tissues were sectioned, and randomized and blinded for histopathological examination. A pathology score of 0-3 was assigned to each sample, based on severity of tissue damages, with higher score reflecting the more severe pathology. A score of 1 was attributed to lung sections that revealed histopathology findings in less than 25% of the section. Similarly, if greater than 25% but less than 50% of the parenchyma was involved, a score of 2 was assigned. A score of 3 was designated to those sections where more than 50% of the total section was affected. Sham vaccinated hamsters inoculated with SARS-COV-2 revealed widespread lung histopathology which resemble the reports of severe pneumonia detected in COVID-19 patients (FIG. 15b). Lungs from naïve hamsters were histologically unremarkable. Similar lesions could be seen in lung samples from the 0.15 μg dose group of single vaccination, which was scored as 3 in blind examination. On the contrary, the lung samples from the 13.5 μg dose group of single vaccination revealed no such lesions, similar to that of health control, and both were scored as 0 (FIG. 15c).

Lung pathology was markedly attenuated in hamsters that received either one or two doses of the LNP formulation of Example 5, and there appeared to be a dose-dependent effect at both 4 and 7 DPI (FIG. 15b). While a single vaccination of 1.5, 4.5 and 13.5 μg substantially attenuated pathology caused by infection, the two-dose vaccination of 1.5, 4.5 and 13.5 μg provided almost complete protection against pathology. The very low dose level of 0.15 μg showed no protection when used in a single-dose regimen but some marginal protection in a two-dose vaccination regimen.

To assess whether immunization with the LNP formulation of Example 5 could impact viral infection in hamsters, viral subgenomic mRNA (sgRNA) from lung and nasal samples by RT PCR were measured. Lung and nasal samples of half the group (n=4) were collected at either 4 or 7 DPI and total RNA was processed for detection of sgRNA by RT-PCR (FIG. 15d). For lung samples collected at 4 and 7 DPI, the sham vaccinated group yielded about 108 and 105 copies per gram tissues, respectively, while those receiving the 13.5 μg two-dose regimen were below the level of detection at both time points. The lung samples from those receiving the 1.5 μg and 4.5 μg two-dose regimens had a nearly 3 log reduction in viral sgRNA copies at 4 DPI and were below detection at 7 DPI. For the lung samples from those receiving the 1.5, 4.5 and 13.5 μg single-dose vaccination, the viral loads at 4 DPI were not different from those of the sham vaccinated group while the loads at 7 DPI were below the threshold of detection. Notably, the lung samples from the 0.15 μg receiving one-dose or two-dose regimens had similar or even higher viral load as compared to those of the sham vaccinated group at either 4 or 7 DPI. However, the viral loads (sgRNA) were more diverse at 4 DPI among all groups, with one or two animals testing negative in most groups. The only group that achieved clearance of viral sgRNA in nasal samples at 7 DPI was the 13.5 μg two-dose vaccination group.

This example demonstrates that the immunogenic composition prepared in Example 5 can reduce viral infection of the lung and prevent lung pathology in an animal model of COVID-19. Immunization with the immunogenic composition prepared in Example 5 may have an impact on transmission due to shortened duration and lower loads of viral shedding from the upper respiratory tract.

### Example 13. Preparation of mRNA-Encapsulating Lipid Nanoparticles

An mRNA comprising an optimized nucleotide sequence encoding a full-length SARS-COV-2 S protein that has been modified relative to naturally occurring SARS-COV-2 spike protein to remove the furin cleavage site, to mutate residues 986 and 987 to proline and which contains the L18F, D80A, D215G, ΔL242, ΔA243, ΔL244, K417N, E484K, N501Y, D614G and A701V mutations (South African variant 2+D614G) was synthesized in vitro. The mRNA was prepared using a template plasmid comprising the sequence SEQ ID NO: 166 operable linked to an RNA polymerase promoter sequence.

Template-dependent RNA synthesis of unmodified nucleotides yielded a polynucleotide with the nucleic acid sequence of SEQ ID NO: 172 which comprises the optimized nucleic acid sequence of SEQ ID NO: 173. In a multi-step, enzyme-catalyzed process, the final mRNA product was synthesized, which was purified to remove enzyme reagents and prematurely aborted synthesis products (“shortmers”).

The final mRNA had the structural elements shown in mRNA construct 2 in Table 4. The SARS-COV-2 S protein coding sequence is flanked by 5′ and 3′ untranslated regions (UTRs) of 140 and 105 nucleotides, respectively. The mRNA also contains a 5′ cap structure consisting of a 7-methyl guanosine (m7G) residue linked via an inverted 5′5′ triphosphate bridge to the first nucleoside of the 5′ UTR, which is itself modified by 2′Oribose methylation. The 5′ cap is essential for initiation of translation by the ribosome. The entire linear structure is terminated at the 3′ end by a tract of approximately 100 to 500 adenosine nucleosides (polyA). The polyA region confers stability to the mRNA and is also thought to enhance translation. All of these structural elements are naturally occurring components which are required for the efficient translation of the SARS-CoV-2 spike mRNA.

The purified mRNA was encapsulated in lipid nanoparticles (LNPs) comprising 40% cKK-E10, 30% DOPE, 28.5% Cholesterol and 1.5% DMG-PEG-2K (molar ratios). The final mRNA-LNP formulation was an aqueous suspension.

### Example 14. Neutralizing Antibody Response Effective Against Variant Strains of SARS-COV-2

This example demonstrates that non-human primates (NHPs), which previously had been immunized with two doses of the LNP formulation of Example 5, mount an effective neutralizing antibody response against the SARS-COV-2 S protein derived from the original Wuhan strain as well as naturally occurring variants of the SARS-COV-2 S protein observed in South Africa, Japan/Brazil and California, and an S protein derived from SARS-COV-1 in response to exposure with an immunogenic composition of LNP-encapsulated mRNA comprising an optimized nucleotide sequence encoding a SARS-COV-2 S protein that has been modified relative to naturally occurring SARS-COV-2 S protein to remove the furin cleavage site to mutate residues 986 and 987 to proline and which contains the L18F, D80A, D215G, ΔL242, ΔA243, ΔL244, K417N, E484K, N501Y, D614G and A701V mutations of a South African variant (South African variant 2+D614G) of SARS-COV-2. The immunogenic composition was prepared as described in Example 13.

A non-human primate (NHP) model (cynomolgus monkeys) was used to investigate whether the original antigen specificity towards the original Wuhan strain, which was induced by the mRNA vaccine described in Example 5 (encoding a prefusion-stabilized Wuhan variant of the SARS-COV-2 protein), could be overcome by subsequent immunization with an mRNA vaccine comprising an optimized nucleotide sequence encoding a prefusion-stabilized South African (SA) variant of the SARS-COV-2 S protein, either alone or in combination of the mRNA vaccine of Example 5 (Wuhan), in order to elicit a broad immune response targeting different circulating variants of SARS-COV-2 and an S protein derived from SARS-COV-1. Cynomolgus monkeys (n=4) were immunized twice three weeks apart (Day 0 and Day 21) with either 15 μg, 45 μg or 135 μg each of the LNP formulation prepared in Example 5. On Day 315 animals were randomized, distributed in two groups and immunized. Group 1 was immunized with an mRNA vaccine described in Example 13, which contained mutations derived from a South African variant of SARS-COV-2 (SA alone). Group 2 was immunized with a formulation that contained the original mRNA vaccine from Example 5 plus the variant given to Group 1 (Wuhan+SA). Both Group 1 and Group 2 received a total mRNA dose of 10 μg. The study was designed to evaluate whether a bivalent immunogenic composition (Wuhan+SA) was required to broaden the antigen response, or whether a monovalent immunogenic composition comprising a SARS-CoV-2 S protein derived from a non-Wuhan variant (SA alone) was sufficient to broaden the antigen response.

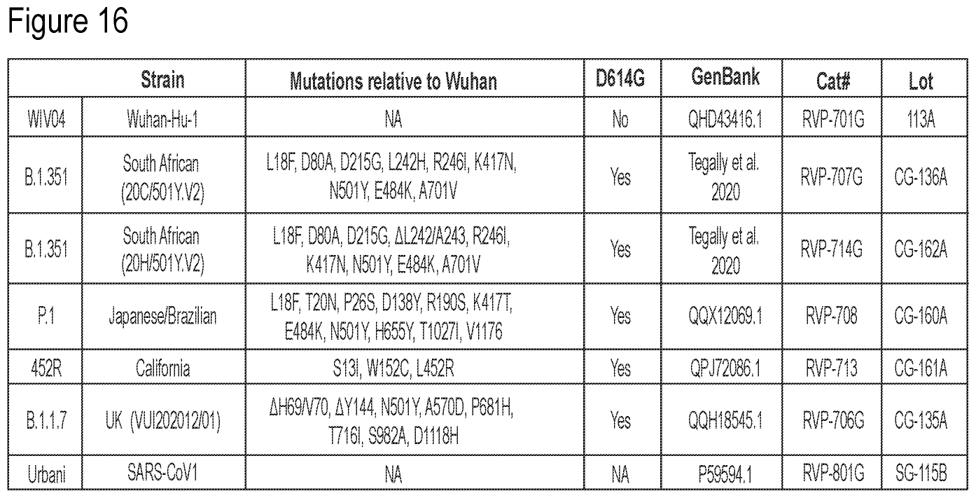

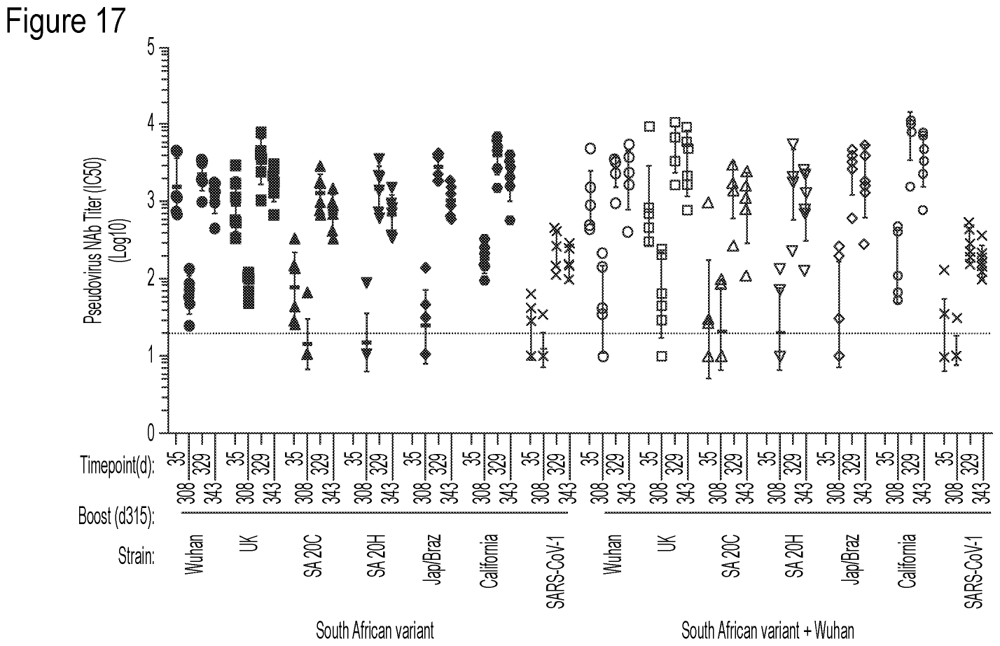

Serum samples from pre-immunized and pre-boost animals (Day 4, Day 308) as well as samples collected on Day 14, Day 21, Day 28, Day 35, Day 42, Day 90, Day 308 and Day 329 were tested in a Wuhan S-protein-expressing pseudovirus (PsV) neutralization assay. Serum samples collected on Day 35, Day 308 and Day 329 were tested in pseudovirus (PsV) neutralization assays. The tested PsVs expressed an S protein derived from SARS-COV-2 strains Wuhan, South African (SA 20C and SA 20H), Japan/Brazil (Jap/Braz) or California, or an S protein derived from a SARS-COV-1 strain, as shown in FIG. 16. Serum samples were diluted in medium (FluoroBrite phenol red free DMEM+10% FBS+10 mM HEPES+1% PS+1% Glutamax) and heat-inactivated at 56° C. for 30 minutes. A further, 2-fold, 11-point, serial dilution series of the heat-inactivated serum was performed in medium. Diluted serum samples were mixed with reporter virus particle (RVP)-GFP (Integral Molecular) diluted to contain ˜300 infectious particles per well and incubated for 1 hour at 37° C. 96-well plates of ˜50% confluent 293T-hsACE2 clonal cells in 75 μL volume were inoculated with 50 μL of the serum+RVP mixtures in singleton and incubated at 37° C. for 72h. At the end of the incubation, plates were scanned on a high-content imager and individual GFP expressing cells were counted. The inhibitory dilution titer (ID50) was reported as the reciprocal of the dilution that reduced the number of virus plaques in the test by 50%. ID50 for each test sample was interpolated by calculating the slope and intercept using the last dilution with a plaque number below the 50% neutralization point and the first dilution with a plaque number above the 50% neutralization point (ID50 Titer=(50% neutralization point-intercept)/slope). The results are summarised in FIG. 17.

As can be seen from FIG. 17, in both groups of NHPs booster immunization with an mRNA vaccine comprising an optimized nucleotide sequence encoding a fusion-stabilized South African variant of the SARS-COV-2 S protein about 9 months after the original 2-dose prime-boost immunization resulted in high neutralization potencies against Wuhan PsV, which expressed the SARS-COV-2 S protein of the original Wuhan strain. These data suggest that exposure to an mRNA vaccine encoding a South African variant of the SARS-COV 2 S protein boosts the neutralizing antibody response against the SARS-COV-2 S protein encoded by the original mRNA vaccine. Exposure to a mixture of the mRNA vaccine encoding the prefusion stabilized South African variant of the SARS-COV-2 S protein and the original mRNA encoding a prefusion stabilized S protein derived from the Wuhan strain was no more effective in boosting a neutralizing antibody response against the S protein of the original Wuhan strain than exposure to only the mRNA vaccine encoding the prefusion stabilized South African variant of the SARS-CoV-2 S protein.

Interestingly, immunization with an mRNA vaccine encoding the prefusion stabilized South African variant of the SARS-COV-2 S protein also resulted in high neutralization potencies against all other tested PsV, which expressed a naturally occurring variant of the SARS-CoV-2 S protein observed in South Africa and naturally occurring variants of the SARS-COV-2 S protein observed in Japan/Brazil and California. Surprisingly, the antigen response was so broad that PsVs expressing the S protein of SARS-COV-1 were also effectively neutralized by the NHP test sera. This was unexpected since the S protein of SARS-COV-1 is only 76% identical to the S protein of SARS-COV-2 Wuhan.

As can be seen from FIG. 17, in most instances the neutralizing antibody response was as effective against a variant S protein as against the S protein derived from the original Wuhan strain. Moreover, the magnitude of the neutralizing antibody response observed after booster immunization with an mRNA vaccine encoding a prefusion stabilized South African variant of the SARS-COV-2 S protein was similar or greater to the neutralizing antibody response induced at Day 35 in response to the original prime-boost immunization with the mRNA vaccine of Example 5.

These data demonstrate that subjects who have been previously immunized with a vaccine that elicits neutralizing antibodies against the S protein of SARS-COV-2 Wuhan and who are subsequently administered an mRNA vaccine comprising an optimized nucleotide sequence of the invention that encodes a prefusion stabilized South African variant of the SARS-COV-2 S protein are able to mount a broad neutralizing antibody response effective against a wide variety of S protein variants and therefore should be effectively protected against COVID-19 infections caused by naturally occurring variants of the original SARS-COV-2 Wuhan strain, as well as other β-coronaviruses, in particular those expressing a spike protein which binds to angiotensin-converting enzyme 2 (ACE2), such as SARS-COV-1.

1. A nucleic acid comprising an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline, wherein the optimized nucleotide sequence consists of codons associated with a usage frequency which is greater than or equal to 10%; wherein the optimized nucleotide sequence:
(i) does not contain a termination signal having one of the following nucleotide sequences:
5′-X₁ATCTX₂TX₃-3′, wherein X₁, X₂ and X₃ are independently selected from A, C, T or G; and 5′-X₁AUCUX₂UX₃-3′, wherein X₁, X₂ and X₃ are independently selected from A, C, U or G;
(ii) does not contain any negative cis-regulatory elements and negative repeat elements; and
(iii) has a codon adaptation index greater than 0.8;
wherein, when divided into non-overlapping 30 nucleotide-long portions, each portion of the optimized nucleotide sequence has a guanine cytosine content range of 30%-70%.

2. The nucleic acid of claim 1, wherein the optimized nucleotide sequence does not contain a termination signal having one of the following sequences: TATCTGTT; TTTTTT; AAGCTT; GAAGAGC; TCTAGA; UAUCUGUU; UUUUUU; AAGCUU; GAAGAGC; UCUAGA.

3. The nucleic acid of claim 1, wherein the full-length SARS-CoV-2 spike protein encoded by the optimized sequence further contains the L18F, D80A, D215G, L242-, A243-, L244-, K417N, E484K, N501Y, D614G and A701V mutations.

4. The nucleic acid of claim 1, wherein the nucleic acid is mRNA.

5. (canceled)

6. The nucleic acid of claim 1, wherein the optimized nucleotide sequence encodes an amino acid sequence comprising SEQ ID NO:11, optionally wherein the optimized nucleotide sequence has the nucleic acid sequence of SEQ ID NO: 44 or SEQ ID NO: 148; or wherein the optimized nucleotide sequence encodes an amino acid sequence comprising SEQ ID NO: 167, optionally wherein the optimized nucleotide sequence has the nucleic acid sequence of SEQ ID NO: 166 or SEQ ID NO: 173.

7.-16. (canceled)

17. A pharmaceutical composition comprising i) the nucleic acid of claim 1 and ii) a lipid nanoparticle, wherein the nucleic acid is encapsulated in the lipid nanoparticle.

18. (canceled)

19. The pharmaceutical composition of claim 17, wherein the lipid nanoparticle comprises a cationic lipid, a non-cationic lipid, a cholesterol-based lipid, and a PEG-modified lipid.

20.-35. (canceled)

36. The nucleic acid of claim 4, wherein the mRNA comprises an mRNA construct consisting of the following structural elements:
(i) a 5′ cap with the following structure: 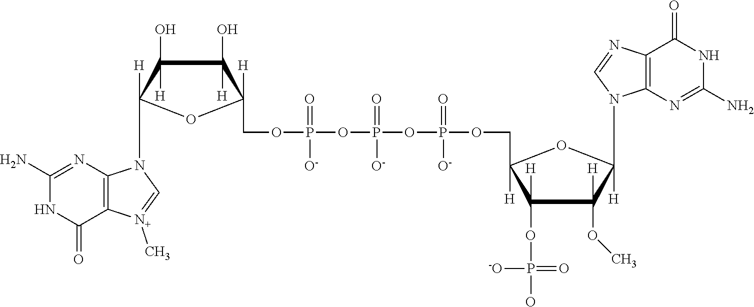
(ii) a 5′ untranslated region (5′ UTR) having the nucleic acid sequence of SEQ ID NO: 144;
(iii) a protein coding region having the nucleic acid sequence of SEQ ID NO: 148;
(iv) a 3′ untranslated region (3′ UTR) having the nucleic acid sequence of SEQ ID NO: 145; and
(v) a polyA tail.

37. The nucleic acid of claim 4, wherein the mRNA comprises an mRNA construct consisting of the following structural elements:
(i) a 5′ cap with the following structure: 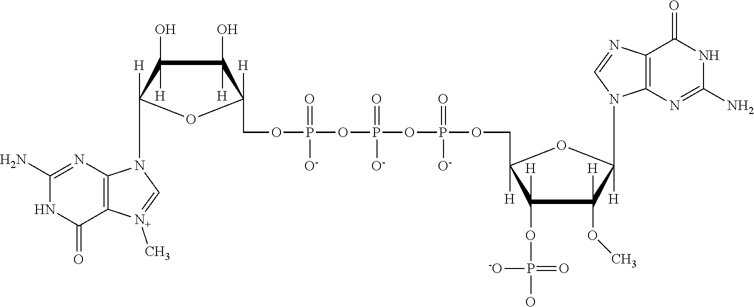
(ii) a 5′ untranslated region (5′ UTR) having the nucleic acid sequence of SEQ ID NO: 144;
(iii) a protein coding region having the nucleic acid sequence of SEQ ID NO: 173;
(iv) a 3′ untranslated region (3′ UTR) having the nucleic acid sequence of SEQ ID NO: 145; and
(v) a polyA tail.

38.-43. (canceled)

44. The pharmaceutical composition of claim 17 comprising the mRNA construct of claim 36 and/or the mRNA construct of claim 37.

45. The immunogenic composition according to claim 44 comprising between 5 μg and 200 μg of the mRNA construct(s).

46.-60. (canceled)

61. An immunogenic composition comprising at least two nucleic acids, wherein
1. the first nucleic acid comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline; and
2. the second nucleic acid comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline and further contains the L18F, D80A, D215G, L242-, A243-, L244-, K417N, E484K, N501Y, D614G and A701V mutations.

62. The immunogenic composition according to claim 61, wherein the first nucleic acid comprises an optimized nucleotide sequence which encodes an amino acid sequence comprising SEQ ID NO: 11, optionally wherein the optimized nucleotide sequence has the nucleic acid sequence of SEQ ID NO: 44 or SEQ ID NO: 148.

63. The immunogenic composition according to claim 61 or 62, wherein the second nucleic acid comprises an optimized nucleotide sequence that encodes an amino acid sequence comprising SEQ ID NO: 167, optionally wherein the optimized nucleotide sequence has the nucleic acid sequence of SEQ ID NO: 166 or SEQ ID NO: 173.

64. (canceled)

65. The immunogenic composition according to claim 61, wherein the optimized nucleotide sequence of the first nucleic acid has the nucleic acid sequence of SEQ ID NO: 148, and wherein the optimized nucleotide sequence of the second nucleic acid has the nucleic acid sequence of SEQ ID NO: 173.

66. (canceled)

67. (canceled)

68. The immunogenic composition according to claim 61, wherein the at least two nucleic acids are encapsulated in the same lipid nanoparticle, or in separate lipid nanoparticles.

69. (canceled)

70. The immunogenic composition of claim 68, wherein the lipid nanoparticle comprises a cationic lipid, a non-cationic lipid, a cholesterol-based lipid and a PEG-modified lipid.

71. The immunogenic composition according to claim 70, wherein the cationic lipid is selected from cKK-E12, cKK-E10, OF-Deg-Lin and OF-02; the non-cationic lipid is selected from DOPE and DEPE; the cholesterol-based lipid is cholesterol; and the PEG-modified lipid is DMG-PEG-2K.

72. The immunogenic composition according to claim 71, wherein the lipid nanoparticle comprises cKK-E10, DOPE, cholesterol and DMG-PEG2K at the molar ratios 40:30:28.5:1.5.

73. The immunogenic composition of claim 61 comprising at total of 7.5 μg, 15 μg, 45 μg or 135 μg of the at least two nucleic acids.

74.-77. (canceled)

78. A method of treating or preventing an infection caused by a β-coronavirus, said method comprising administering to a subject an effective amount of the pharmaceutical composition of claim 17, or the immunogenic composition of claim 61.

79. (canceled)

80. The method of claim 78, wherein the β-coronavirus is SARS-COV-2.

81.-89. (canceled)

90. A method of treating or preventing an infection caused by a β-coronavirus, said method comprising administering to a subject an effective amount of an immunogenic composition comprising an mRNA construct, wherein said mRNA construct comprises an optimized nucleotide sequence encoding a full-length SARS-COV-2 spike protein which has been modified relative to naturally occurring full-length SARS-COV-2 spike protein of SEQ ID NO: 1 to remove the furin cleavage site and to mutate residues 986 and 987 to proline and further contains the L18F, D80A, D215G, L242-, A243-, L244-, K417N, E484K, N501Y, D614G and A701V mutations.

91. The method of claim 90, wherein the optimized nucleotide sequence encodes an amino acid sequence comprising SEQ ID NO: 167, optionally wherein the optimized nucleotide sequence has the nucleic acid sequence of SEQ ID NO: 173.

92.-108. (canceled)
